SEQUENCE LISTING (156 sequences)

SEQ ID NO: 1 - DNA, 6595 bp, synthetic construct
  source 1..6595: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccatgtccgtgctgacgaagtggctgggcctgccctccttcctgttctccgtgttcgtgttctactggtccctgcccatcttcgccatcctgtaccgcatccgcttcgcctccctgggcaagcgcaacgacatgctggactgggcccgcgccctggtggcctacttccgcgtgaccctgctccaggcgggcgagcacaccctgtacaagggcggcccctgcctgtacctgtgcaaccaccgctcctgggccgacttcttcatcgacgcctacctgaccgagggccgcgccgccctgatgtcccgctggctggtgtacttcgtgttccccgtgttctgcacctcctgcatgatcctgaagggcatcgtgctgttcaagcgcggcaccatcgccgacaaggaggccttcaacgcctggctggaccagaccctgggctcctcccacgtgcccggcctgctggtgtaccccgagggccaccgctccaccaagcccgcctccctgcccctgaagcgcggcatgctgcactacgcccactcccgcaagctgcccgtgcagatcgtggtgacccgcggcaaggacgaggtgctgtccgagaagtcccagtccgtgcacttcggccgcacctgcgtgaccaccttctccaaggtgctgaagtccgccgactaccccaacttcgaggccttcttcaccgacctccaggccacctgggactcctgctgggccgccacctacggcctggaggacctgaagaacgtgccccgcttctccatgcccggcccccaggcctactcctactcctcctccatgtgggtgcagcaactggcgatcaccctggtgtccatcctggtgttcgcgggcgtgtgctacggctcctggcgcggcctggccgccgccctggccgccacgggcgccgcccagcaggtggtggccctggtgctggccgcctgggtgggctcctccgtgctgcgctccttcctgtagttaattaagcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 2 - DNA, 6616 bp, synthetic construct
  source 1..6616: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccatggcccgcaagtcctccctggcccaggccgccatcgagcgcaagcccgtgctgctgcgcccccagctcaacgtgccccgcatgtccggcatcaccgccctgcccatggagcgccgccccctgcccgtggccccctccccctccgccaagcccgagctgcccgcccgctccgccctggtgtgccacgccgccgccgcctccgtgcccctgcccaactccgactccgccccccagcccaacgtgctgctggccaagatccgcgccatcatgttcttcgcctggtccttcctgctgtccctgcccctgttcgtgaccatgatggtgatggcccccctggtgctggccttcgacaagtaccgccgcctggcccagcacttcgtgaacaacctgtgggcctgcgcctccaccgcccccttctacaaggtgaccatcatcggccgcgagaacctgcccccccccgacaagcccgtggtgtacgtggccaaccaccagtccttcctggacatctactccctgttccacctccagcgccccttcaagttcatctccaagacctccaacttcctgatccccatcatcggctggtccatgttcctgacgggccacgtgatgatcaaccgcgtggaccgccgctcccagctcaagtgcctccagcagtgccgcgacctgctggccgagggcgcccccgtgctgttcttccccgagggcacccgctccctggactgcaagatggcgggcttcaagaagggcgccttctccgtggccgccaaggcgggcgtggaggtggtgcccatcaccctgctgggcacgggctccctgatgccctccggcaaggagtcccagctccgccccggccaggtgaccatcgtggtgcacaaggccctgccccccaacaagaacgccgaccagctctgcgacgccgcccgccaggccgtggcctcctccctgccccccgagctggtgggctccgcgaccgagatggcccccgacgagcagtagttaattaagcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 3 - DNA, 6652 bp, synthetic construct
  source 1..6652: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccatggacgtggcctccgcccgcggcgtgtcctcccaccccccctactactccaagcccatctgctcctcccagtcctccctgatccgcatccccatctccaagggctgctgcttcgcccgctcctccaacctgatcacctccctgcacgccgcctcccgcggcgtgacccgccgcacctccggcgtgcagtggtgctaccgctccatccgcttcgaccccttcaaggtgaacgacaagaactcccgcaccgtgaccgtgcgctccgacctgtccggcgccgccacccccgagtccacctaccccgagcccgagatcaagctgtcctcccgcctgcgcggcatctgcttctgcctggtggcgggcatctccgccatcgtgctgatcgtgctgatgatcatcggccaccccttcgtgctgctgttcgaccgctaccgccgcaagttccaccacttcatcgccaagctgtgggcctccatctccatctaccccttctacaagaccgacatccagggcctggagaacctgccctcctccgacaccccctgcgtgtacgtgtccaaccaccagtccttcctggacatctacaccctgctgtccctgggccagtcctacaagttcatctccaagacgggcatcttcgtgatccccgtgatcggctgggccatgtccatgatgggcgtggtgcccctgaagcgcatggacccccgctcccaggtggactgcctgaagcgctgcatggagctggtgaagaagggcgcctccgtgttcttcttccccgagggcacccgctccaaggacggccgcctgggccccttcaagaagggcgccttcaccatcgccgccaagacgggcgtgcccgtggtgcccatcaccctgatgggcacgggcaagatcatgcccacgggctccgagggcatcctgaaccacggcgacgtgcgcgtgatcatccacaagcccatctacggctccaaggccgacgtgctgtgcgaggaggcccgcaacaagatcgccgagtccatgaacctgctgtcctagttaattaagcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 4 - DNA, 6955 bp, synthetic construct
  source 1..6955: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccatgtccggccccgccgacgcccgcggccccgcccgcgtgtccgacaccatcatcggcgcccccgaccgccaccacgtgggcctgaccatcctggtcgccctgctgctgtccgccttctgcttctgcgagttcttcatgctgcccgtggccatggccctgctgcccgtgcccggcaccacctcccgcaagctgctgcgccgctgggagggcctggtggccaagacctggctgtccttcggcggctggctgctggagaacgtgggcggcgtgaagctgatcctgacgggcgactccatcctgcccggcgaccgcgtgctgatcctgtccaaccaccgcacccgcatcgactggatgttcctgtggtgctgggccgcccgcttcgacctgctgtcctcctaccgcgtgatcctgaagtcctccctgcgcaccttcccctggtggggctggggcatgtccctgtgcctgttccccttcatccaccgcggctccaacaaccgcgacgccgacctgacccgcatcgagcacatctgccagtacctgtccgagctgggcgtgcccaactccctgatcctgttccccgagggcaccgacctgtcccagtccaaccagaagcgcgaccgcgactacgccctggccaagaacctgcccatctaccacaacgtgctgcacccccgctccggcgccttcatcgcctccctgaccgccatgcgcccccacctggacgccgtggtggacctgaccatcggctacgtggactacacccccggcgagcgcccctccgagctgtccctgctgaagggccgcctgccccacgaggtgcacatcaacatgaagcgctgggacatcaagaccacccccctgctgcgcgagaacccctccgaccgcgccgagcagttcctgcgcgagtccttcgaccgcaaggaggccctgctgaccgccttctactccaacaacaccgacacctccggcggcggcctgaagcagacctcctccctgctggtgtcctcctcctcctcccccttctccaactcctccgcccccctgggcttcccctccttctcctccccctcgacctccgtggcctcctcctacaacgagcccgtgtacgtgcacaacgcccactggacctacggcaagggcctgctgggcacgggcatcgccatggccctcgtgctgatgctggcctgctgcctgcccgccgtggccttctgcggctactccatcgtggtgttcctgctgttcgtgaccgtgaactccgtgtgggagggcttcgacaccctggagctgaacctggccgtgtggtggggcggccaggcgggccagaccaagcaggagtagttaattaagcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 5 - DNA, 6295 bp, synthetic construct
  source 1..6295: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccatggactccgagatcaaccaccgcggcggcctgtccgccccccgcccccgcgagacctccctgaacctggccctgtaccgcggcctgaagtggggcgtggtgcgccccctgctgcacggcctgttccaggcccaggtgtacggccaggagctggtgcccacccgcggccccgccctggtggtgtccaaccacgcctcctacttcgacccccccttcctgtcctgcgccatggcccgccccgtggccttcatggccaaggaggagctgttcaacgtgcccctgctgggccccgccatccgcctgtacggcgcctaccccgtgaagcgcggctccggcgaccgcggcgccctgcgcgccgccctgaccgccctgggcgacggctggctggtgggcgtgttcctggagggcacccgcaccaaggacggccgcatccaccagcccaagctgggcgccgccatgatcgccgccaaggcccaggtgcccatcatccccgtgtccctgggcggcgtggagcagatcttccagcccggctccccctggccccaccccgtgcccctgaccatccgcatcggcaaggccatcgccccccccgtgaagaaccgcaagcccgagctggaggccgtgaccaaggcctgccaggcccagatccacgagatgctggacctgggccgcgactagttaattaagcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 6 - DNA, 6355 bp, synthetic construct
  source 1..6355: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccatgctgtacatcttccgcctgatcatcaccgtgatctactccatcctggtgtgcgtgttcggctccatctactgcctgttctccccccgcaaccccaagcacgtcgccaccttcggccacatgttcggccgcctggcccccctgttcggcctgaaggtggagtgccgcaagcccaccgacgccgagtcctacggcaacgccatctacatcgccaaccaccagaacaactacgacatggtgaccgcctccaacatcgtgcagccccccaccgtgaccgtgggcaagaagtccctgctgtggattcccttcttcggccagctctactggctgacgggcaacctgctgatcgaccgcaacaaccgcaccaaggcccacggcaccatcgccgaggtggtcaaccacttcaagaagcgccgcatctccatctggatgttccccgagggcacccgctcccgcggccggggcctgctgcccttcaagacgggcgccttccacgccgccatcgccgcgggcgtgcccatcatccccgtgtgcgtgtccaccacctccaacaagatcaacctgaaccgcctgcacaacggcctggtgatcgtggagatgctgccccccatcgacgtgtcccagtacggcaaggaccaggtgcgcgagctggccgcccactgccgctccatcatggagcagaagatcgccgagctggacaaggaggtggccgagcgcgaggccgcgggcaaggtgtagttaattaagcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 7 - DNA, 7173 bp, synthetic construct
  source 1..7173: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggaggtaccccgctcccgtctggtcctcacgttcgtgtacggcctggatcccggaaagggcggatgcacgtggtgttgccccgccattggcgcccacgtttcaaagtccccggccagaaatgcacaggaccggcccggctcgcacaggccatgacgaatgcccagatttcgacagcaaaacaatctggaataatcgcaaccattcgcgttttgaacgaaacgaaaagacgctgtttagcacgtttccgatatcgtgggggccgaagcatgattggggggaggaaagcgtggccccaaggtagcccattctgtgccacacgccgacgaggaccaatccccggcatcagccttcatcgacggctgcgccgcacatataaagccggacgccttcccgacacgttcaaacagttttatttcctccacttcctgaatcaaacaaatcttcaaggaagatcctgctcttgagcaactcgtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacaacttattacgtattctgaccggcgctgatgtggcgcggacgccgtcgtactctttcagactttactcttgaggaattgaacctttctcgcttgctggcatgtaaacattggcgcaattaattgtgtgatgaagaaagggtggcacaagatggatcgcgaatgtacgagatcgacaacgatggtgattgttatgaggggccaaacctggctcaatcttgtcgcatgtccggcgcaatgtgatccagcggcgtgactctcgcaacctggtagtgtgtgcgcaccgggtcgctttgattaaaactgatcgcattgccatcccgtcaactcacaagcctactctagctcccattgcgcactcgggcgcccggctcgatcaatgttctgagcggagggcgaagcgtcaggaaatcgtctcggcagctggaagcgcatggaatgcggagcggagatcgaatcactgaagaatgggaggcaggtgctgctgattacgagtgtggaaaagaaagggggcagagagccgtcctcagatccgaccactatgcaggtagccgctcgcccgtgcccgcctggctgaatattgacacatgcccatcaaggcaggcaggcatttctgtgcatgcaccaaacccataatcttcgaaaacacacagcatgtaccaacgcacgcgtaaaagttggggtgctgccagtgcgtcatgccaggcatgatgtgctcctgcacatcggccatgatctcctccatcgtctcgggcgtctccggcgcctggtccgggagccgttccgccagatacccagacgccacctccgacctcacggggtacttttcgagcgactgtcggtagtcgacgatcgcgtccaccatggagtagccgaggcgccggaactggcgtgacggagggagaacagggggggttgatgatcacacgccagtctcacaacgcatgtaagacccgttggattatgagtataatcatgcattactagttgggtgagcgccaggcataaggcacaccgacgtcgatggcacgagcaactcccgcatcatatttcctattgtcctaacgccaagccggtcaccacccgcatgctcgtactacagcgcacgcaccgcttcgtgatccaccgggtgaacgtagtcctcgacggaaacatctggttcgggcctcctgcttgcactcccgcccatgccgacaacctttctgctgttaccacgacccacaatgcaacgcgacacgaccgtgtgggactgatcggttcactgcacctgcatgcaattgtcacaagcgcttactccaattgtattcgtttgttttctgggagcagttgctcgaccgcccgcgtcccgcaggcagcgatgacgtgtgcgtggcctgggtgtttcgtcgaaaggccagcaaccctaaatcgcaggcgatccggagattgggatctgatccgagtttggaccagatccgccccgatgcggcacgggaactgcatcgactcggcgcggaacccagctttcgtaaatgccagattggtgtccgatacctggatttgccatcagcgaaacaagacttcagcagcgagcgtatttggcgggcgtgctaccagggttgcatacattgcccatttctgtctggaccgctttactggcgcagagggtgagttgatggggttggcaggcatcgaaacgcgcgtgcatggtgtgcgtgtctgttttcggctgcacgaattcaatagtcggatgggcgacggtagaattgggtgtggcgctcgcgtgcatgcctcgccccgtcgggtgtcatgaccgggactggaatcccccctcgcgaccatcttgctaacgctcccgactctcccgaccgcgcgcaggatagactcttgttcaaccaatcgacaatgtggggcctgctgtggaagcacatgaccctgcgcctgctgttcgtgtccgtgtgcctggtgacctcctccatcggcgcgaacatgatggccctggccctgttcctggtgatccgccccttctcccgctccctgtaccgccgcctggtgtcccagtgcgtggcctgcatgtggatcgacgccctgtccctgctgctgcccggcaccaacatccacatcgccgccgactccgacatgcccgacggcatcaccgcgggcatcgtggtggccaaccaccagtacgagggcgactggtggttcatgctgatggtggcccgcttcctgggcctgcacggcaacgtgaagatcatcgtgcgcgagggcctgaagaagatccccctgctgggctggctggtgcgcctggtggagtaccccttcatctcctcctcctggtccctgtcccgcaccaacctgttcggcctgctgcgctccttcaacgccgacgacttccccgtgctgctgttccagttccccgagggcgaccgcatcgacgccaaggtgcgccagcagtccctggccttcgccgccaaggagcagcgcccccacctgctgcacgtgctgctgccccgcaccacgggcttcaacacctgcatcgaggccctgcgcacctcccacccccccgtgtacgacatgaccatcgccatccccggcaccacgggccagccctcctcctcctcctcctcctcctccccctccgccgccgccgccgccgcctccccctccgcgggcgccgccgccgccgccgccgccgcctccaacacctccaccaacccctcctccaaccccccctcctcctcctccaccgacacctcccccgccgccgccgccgccgccgccgccgacgccgccgccgccgagtcccacgacgcctccctgttccacaccttcctgcgcttctgcaacggcgagggcccccgcgacgtgcacatccgcctgaagcgctactccctgaacgacgtgctggccgacccccactggctggacaccaagtgggccgagaaggaccgcgtgctgacctacttctcccgccacgcctgcttccccgcccccctgcccccccacatggccaacggcggcggctccacccagacccacggcggccggggcggcccccgcggccccgcgggctcccacgccgtggtgggcgccgcgggcaacgcccagtacctgcgctccttcaactcccgcaagttcaaggccgagacctccttcctggccctggcccgcctgctgctgacccccctgtgcctgcccctgctgctgctgatggcctgccccctgatgaccctgtacgtgtgcgtgtccaccgtgcgccgctggctgggcgaggagatcgtgttctcccccggcggccgggagggcggccgggagggcgacgccgtgtacgagtccgtgaactggtccgtgtgattaattaagcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 8 - DNA, 6529 bp, synthetic construct
  source 1..6529: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccatgtctgtgccgtcgaaggcgctctccctgcctagcttcctgttcagcgtattcgtcttctactggtcccttcctctctttgcaattatctaccgcatccgtttcgctaacgtcggcaagcgcaacgacatgctcgactggtggatggtgtttttcgcgtttcctgtgttctgcacgtccgttctgatcctgcggggtatcgtgctgtttaagcgcggacatatcgccgacaaggagttgctgaagtcggacgcggtggtcgtgatggtcgtgatggttgtggccaccgtcgtgcaggcgtttaacgtctggctggacgcaacgcttcgaaagtccaccgtccctggcctgctcgtgtacccggagggccaccgtagcctgaagccgcgcagcctgcccctcaagcggggaatgttgcacttcgctttcagccgccacctagccgtgcaactggtgatcacgcgtggtaaggaagaggtcatgtctgagaagacgtgctccgcgcggtggggccgcacccttgtcaccaactactcaaaggtgtgcgccgggggccgctggggcggggtaatcaagccaacggatttcgactcgttcgacgcctttttcaacgagattcaaaccacgtgggacgcctgctggcagctctcctaccaccagcccatcgcgggtgtcccccggctgtccctgaaggacgcgcacgagtacgactacccgccctccatgcgctggctgcaactgggcgtgaccctcctttcgatcgtgatcctcgcggccgtcctctacggcagttgggtcgtgctgcgcgcgggcgtgctatcactgggctcctcggttacccaacaggcggtcgtggtcctcgggggcgccgggggctggctgggcctgtcgctgctccgcagcttcttatagttaattaagcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 9 - DNA, 6754 bp, synthetic construct
  source 1..6754: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccatgtcggcgctaacgaaacttctgggtctgccgtccttcctgttttcagtctttgttttctactggtccctacccatctttgccatcttctaccgtatccgcttcgcctcgctgggcaagcgcaacgacatgctggactgggcccgctccctcgtggggttctttcgggtgacgcttttgcaggcaggcgaacagggtctgtacaagggaggcccttgcctatacctgtgcaaccaccgctcttgggccgacttctttgtcgacgcctacctaacggagggtcgcgctgccctgatgagccgctggcttgtgtacttcgtgttcccggtgttctgtacctcgtgcatgatcctgaagggtatagtcctgtttaagcgcgggaccatcgcggacaaggaggcgtttaacgcctggctggaccggaccctgggtgactctcacgtccctggcctcctggtctacccggagggacaccggagcaccaagcctacctcgctgcccttgaagcgaggcatgctgcactacgcgcattcccggaagctgccagtccaggtcatcgtcacgcgcggcaaagacgaggtcatgtccgagaaaggccagtccgttcacttcggccgcacctgcgtcacgaccttctccaaggtcctcaagccagccgactacccgaactttgaggcctttttcgccgacctccaggctacctgggacgcctgctgggcagccacctacggcctgaaggacctgaagggcgtgccgcgcttctcgatgccctctccccaggcttacacgtacagcgccaagacctgggcacagcaactggcgatcacgctcgtgtcgatcgtggtttttgcggccgtctgttacggctcctggcgtggccttgccgcagccctcgctgccacgggtgctgcgcaacaggtccgcggccagcaacagcaacagcaacagcaacagcgccgacggcgcctcagctcctctgggggatgggccgagggtcgcggcgcccaggccgggggacgcctccagtgggacgggggcgactgggggcggtgggtggcgctgcccctgtccctggaggtggtcacggtccttctggctgcgtgggtcggaggcagcgtgctcaggtccttcctgtagttaattaagcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 10 - DNA, 6595 bp, synthetic construct
  source 1..6595: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccatgtcggctctgacgaaacttctggggcttcccagctttctgttctcagtgttcgtgttttactggtccctgccgatttttgccatcctataccgcattcgctttgcgagcctgggcaagcgcaacgacatgctggactgggcgcgcgcgttagtcgggtttttccgcgtgacgctgttacaggctggcgagcagagcctgtacaaagggggtccgtgcctctacctgtgcaaccatcggtcgtgggctgacttctttatcgacgcctacctgacggagggccgggccgcgctgatgtcgcggtggctcgtgtactttgtctttcctgtgttctgcacctcctgcatggtcttgaagggcatcgtcctgttcaagcgcggtacaatcgcggacaaggaggccttcaacgcttggctcgaccggaccctcggcgacagccatgtgcccggcctcctggtctacccggagggacaccgctccaccaagcctgcgtcactgccgctcaagaggggcatgctgcactacgctcactcccgcaagctcccggtgcaggtcattatcacacggggtaaggacgaggtcctgagtgagaagtcccaatcggtgcacttcggccgcacctgcgtgacgaccttttcgaaggtgatcaagccctcggacttccccaacttcgaggcgttctttgccgatctccaggcaacctgggacgcgggctggacggccacctacggcctgaaggacctcaagggcgtcccccgattctccatgcccagcccgcaggcgtacacgtactcaagcaagatgtgggcgcagcaactggcgatcacccttttcagcattccggtctttgcaggcgtctgctacggtagctggcgcgtcttggctgccgcgctggctgccacgggtgccgctcaacaggtggtcaccctcgccttagctggctgggtgggaggctcggtgctgcggagctttctgtagttaattaagcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 11 - DNA, 6592 bp, synthetic construct
  source 1..6592: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccatgagcgtcatccggaaggccctcagcctgcccagcttcctgttctccgtgttcgtcttctactggtccctccccttctttgctattatctaccgtatccgcttcatcaacgtcggcaagcgcaacgacatgctggactgggcgaaggcgctggtcgcgtttttccatgtcaccgtcctcaagcagggcgagatgacgctgtaccgcggtggccgctgtctgtacctgtgcaaccaccgctcctgggccgactttttcatcgacacctacctgaccgagggtcgtgccgcgctcatgtcccgctggctggtgtactttgtgttccccgtcttttgcagctcggtaatcgtcctccgtggcatcgtcctgttcaagcgcggccacattgcgaataaggaggcgtttaacgcatggctggatgtgacgctcggcaagtcgacagtccccggcctcctggtgtacccagagggtcaccgtagcctgaagcccacctccctgcccctgaaacgcggcatgctgcacttcgcctactcccggaagctcccggtccaaattatcgtgacgcgtggcaaggaagaggtgctctccgagaagactctgtcggcgcgctggggcaggacactggtggtcaccttctcgcacgtcctcaagccgtccgactacgattcgttcgatgcgtttttcgatgacatccagcataagtgggacgcttgctggaagtccagctatgacaagcccacggcgggcgtgccagaactctccatgaagggggcgcacgagtacgactacccccgcagcatgtggtggctgcaactgggcatgaccctaatgtcgatcgtcatcttgatcggggtgatgtgggggtcctgggtcgcgctgcgcacgggcctggccgtcctcggggccgcacagcaacccgtggttctgtgcgccgcggtctggatcggcgcctccatgctccgcgccttcttctagttaattaagcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 12 - protein, 332 aa, Chlamydomonas reinhardtii
MARKSSLAQAAIERKPVLLRPQLNVPRMSGITALPMERRPLPVAPSPSAKPELPARSALVCHAAAASVPLPNSDSAPQPNVLLAKIRAIMFFAWSFLLSLPLFVTMMVMAPLVLAFDKYRRLAQHFVNNLWACASTAPFYKVTIIGRENLPPPDKPVVYVANHQSFLDIYSLFHLQRPFKFISKTSNFLIPIIGWSMFLTGHVMINRVDRRSQLKCLQQCRDLLAEGAPVLFFPEGTRSLDCKMAGFKKGAFSVAAKAGVEVVPITLLGTGSLMPSGKESQLRPGQVTIVVHKALPPNKNADQLCDAARQAVASSLPPELVGSATEMAPDEQ

SEQ ID NO: 13 - protein, 325 aa, Chlamydomonas reinhardtii
MSVLTKWLGLPSFLFSVFVFYWSLPIFAILYRIRFASLGKRNDMLDWARALVAYFRVTLLQAGEHTLYKGGPCLYLCNHRSWADFFIDAYLTEGRAALMSRWLVYFVFPVFCTSCMILKGIVLFKRGTIADKEAFNAWLDQTLGSSHVPGLLVYPEGHRSTKPASLPLKRGMLHYAHSRKLPVQIVVTRGKDEVLSEKSQSVHFGRTCVTTFSKVLKSADYPNFEAFFTDLQATWDSCWAATYGLEDLKNVPRFSMPGPQAYSYSSSMWVQQLAITLVSILVFAGVCYGSWRGLAAALAATGAAQQVVALVLAAWVGSSVLRSFL

SEQ ID NO: 14 - protein, 378 aa, Chlamydomonas schloesseri
MSALTKLLGLPSFLFSVFVFYWSLPIFAIFYRIRFASLGKRNDMLDWARSLVGFFRVTLLQAGEQGLYKGGPCLYLCNHRSWADFFVDAYLTEGRAALMSRWLVYFVFPVFCTSCMILKGIVLFKRGTIADKEAFNAWLDRTLGDSHVPGLLVYPEGHRSTKPTSLPLKRGMLHYAHSRKLPVQVIVTRGKDEVMSEKGQSVHFGRTCVTTFSKVLKPADYPNFEAFFADLQATWDACWAATYGLKDLKGVPRFSMPSPQAYTYSAKTWAQQLAITLVSIVVFAAVCYGSWRGLAAALAATGAAQQVRGQQQQQQQQQRRRRLSSSGGWAEGRGAQAGGRLQWDGGDWGRWVALPLSLEVVTVLLAAWVGGSVLRSFL

SEQ ID NO: 15 - protein, 325 aa, Chlamydomonas incerta
MSALTKLLGLPSFLFSVFVFYWSLPIFAILYRIRFASLGKRNDMLDWARALVGFFRVTLLQAGEQSLYKGGPCLYLCNHRSWADFFIDAYLTEGRAALMSRWLVYFVFPVFCTSCMVLKGIVLFKRGTIADKEAFNAWLDRTLGDSHVPGLLVYPEGHRSTKPASLPLKRGMLHYAHSRKLPVQVIITRGKDEVLSEKSQSVHFGRTCVTTFSKVIKPSDFPNFEAFFADLQATWDAGWTATYGLKDLKGVPRFSMPSPQAYTYSSKMWAQQLAITLFSIPVFAGVCYGSWRVLAAALAATGAAQQVVTLALAGWVGGSVLRSFL

SEQ ID NO: 16 - protein, 482 aa, Nannochloropsis oceanica
MWGLLWKHMTLRLLFVSVCLVTSSIGANMMALALFLVIRPFSRSLYRRLVSQCVACMWIDALSLLLPGTNIHIAADSDMPDGITAGIVVANHQYEGDWWFMLMVARFLGLHGNVKIIVREGLKKIPLLGWLVRLVEYPFISSSWSLSRTNLFGLLRSFNADDFPVLLFQFPEGDRIDAKVRQQSLAFAAKEQRPHLLHVLLPRTTGFNTCIEALRTSHPPVYDMTIAIPGTTGQPSSSSSSSSPSAAAAAASPSAGAAAAAAAASNTSTNPSSNPPSSSSTDTSPAAAAAAAADAAAAESHDASLFHTFLRFCNGEGPRDVHIRLKRYSLNDVLADPHWLDTKWAEKDRVLTYFSRHACFPAPLPPHMANGGGSTQTHGGRGGPRGPAGSHAVVGAAGNAQYLRSFNSRKFKAETSFLALARLLLTPLCLPLLLLMACPLMTLYVCVSTVRRWLGEEIVFSPGGREGGREGDAVYESVNWSV

SEQ ID NO: 17 - protein, 445 aa, Nannochloropsis oceanica
MSGPADARGPARVSDTIIGAPDRHHVGLTILVALLLSAFCFCEFFMLPVAMALLPVPGTTSRKLLRRWEGLVAKTWLSFGGWLLENVGGVKLILTGDSILPGDRVLILSNHRTRIDWMFLWCWAARFDLLSSYRVILKSSLRTFPWWGWGMSLCLFPFIHRGSNNRDADLTRIEHICQYLSELGVPNSLILFPEGTDLSQSNQKRDRDYALAKNLPIYHNVLHPRSGAFIASLTAMRPHLDAVVDLTIGYVDYTPGERPSELSLLKGRLPHEVHINMKRWDIKTTPLLRENPSDRAEQFLRESFDRKEALLTAFYSNNTDTSGGGLKQTSSLLVSSSSSPFSNSSAPLGFPSFSSPSTSVASSYNEPVYVHNAHWTYGKGLLGTGIAMALVLMLACCLPAVAFCGYSIVVFLLFVTVNSVWEGFDTLELNLAVWWGGQAGQTKQE

SEQ ID NO: 18 - protein, 344 aa, Brassica napus
MDVASARGVSSHPPYYSKPICSSQSSLIRIPISKGCCFARSSNLITSLHAASRGVTRRTSGVQWCYRSIRFDPFKVNDKNSRTVTVRSDLSGAATPESTYPEPEIKLSSRLRGICFCLVAGISAIVLIVLMIIGHPFVLLFDRYRRKFHHFIAKLWASISIYPFYKTDIQGLENLPSSDTPCVYVSNHQSFLDIYTLLSLGQSYKFISKTGIFVIPVIGWAMSMMGVVPLKRMDPRSQVDCLKRCMELVKKGASVFFFPEGTRSKDGRLGPFKKGAFTIAAKTGVPVVPITLMGTGKIMPTGSEGILNHGDVRVIIHKPIYGSKADVLCEEARNKIAESMNLLS

SEQ ID NO: 19 - protein, 225 aa, Synechocystis sp
MDSEINHRGGLSAPRPRETSLNLALYRGLKWGVVRPLLHGLFQAQVYGQELVPTRGPALVVSNHASYFDPPFLSCAMARPVAFMAKEELFNVPLLGPAIRLYGAYPVKRGSGDRGALRAALTALGDGWLVGVFLEGTRTKDGRIHQPKLGAAMIAAKAQVPIIPVSLGGVEQIFQPGSPWPHPVPLTIRIGKAIAPPVKNRKPELEAVTKACQAQIHEMLDLGRD

SEQ ID NO: 20 - protein, 303 aa, Volvox carteri
MSVPSKALSLPSFLFSVFVFYWSLPLFAIIYRIRFANVGKRNDMLDWWMVFFAFPVFCTSVLILRGIVLFKRGHIADKELLKSDAVVVMVVMVVATVVQAFNVWLDATLRKSTVPGLLVYPEGHRSLKPRSLPLKRGMLHFAFSRHLAVQLVITRGKEEVMSEKTCSARWGRTLVTNYSKVCAGGRWGGVIKPTDFDSFDAFFNEIQTTWDACWQLSYHQPIAGVPRLSLKDAHEYDYPPSMRWLQLGVTLLSIVILAAVLYGSWVVLRAGVLSLGSSVTQQAVVVLGGAGGWLGLSLLRSFL

SEQ ID NO: 21 - protein, 324 aa, Volvox africanus
MSVIRKALSLPSFLFSVFVFYWSLPFFAIIYRIRFINVGKRNDMLDWAKALVAFFHVTVLKQGEMTLYRGGRCLYLCNHRSWADFFIDTYLTEGRAALMSRWLVYFVFPVFCSSVIVLRGIVLFKRGHIANKEAFNAWLDVTLGKSTVPGLLVYPEGHRSLKPTSLPLKRGMLHFAYSRKLPVQIIVTRGKEEVLSEKTLSARWGRTLVVTFSHVLKPSDYDSFDAFFDDIQHKWDACWKSSYDKPTAGVPELSMKGAHEYDYPRSMWWLQLGMTLMSIVILIGVMWGSWVALRTGLAVLGAAQQPVVLCAAVWIGASMLRAFF

SEQ ID NO: 22 - protein, 245 aa, Escherichia coli
MLYIFRLIITVIYSILVCVFGSIYCLFSPRNPKHVATFGHMFGRLAPLFGLKVECRKPTDAESYGNAIYIANHQNNYDMVTASNIVQPPTVTVGKKSLLWIPFFGQLYWLTGNLLIDRNNRTKAHGTIAEVVNHFKKRRISIWMFPEGTRSRGRGLLPFKTGAFHAAIAAGVPIIPVCVSTTSNKINLNRLHNGLVIVEMLPPIDVSQYGKDQVRELAAHCRSIMEQKIAELDKEVAEREAAGKV

SEQ ID NO: 23 - protein, 444 aa, Prototheca moriformis
MAREGLDSVETADGGAKPENGAGEDVREGRSSPSDEDEALPAELEGLYPPEAPIHGPVDAGFAERVMTLQAKHDLAGKGDALLKGTLEETTQLRLRKGEAGSEAGPAGSGSLYELARSSPFIDLQHPIDAYELAKMVFLAPLFLFRALILLVLVASAYLSVMVMFHVLPAGAHGLYPGRDALAQLLIHRCCPILLFLSGFYHIPIKGAENIQKAKEARSIVIFNHLSYVDPAVLLCLMPLSGIAKAGVARIPFIGPLGVALRWLFVERRGSSDRSNPHTLKGDPIDAIRARRADHSAPLLVMAPEATTKAQQCLLKFRRGAFTSGCPVTPVLIRYRFRHFNPAWGLCRSTPLHVYRLLVQPVNHVSLEVLPPYFPDEQETKDWTRFASNVRVRMAQALQAPMVEQGIAEERRLLKAGVTTDLAGSRIIFRRKVAEQATSPRKRD

SEQ ID NO: 24 - protein, 373 aa, Prototheca moriformis
MTLAASIKLALRWPLFLLSMALLYWSVVVGSLGGYIKFLDLKSEQRGDLFAWSVAIKQAFGIRVLRVGKRDVYRGKCMYLVSWGMMAVVFPLFMIALRAMRAVLVFNRGQIPDKEKFNNWIDKELANTPQTGLAVYPEGHRSVLSDSLPLKRGMLHYAYSRKMTVQCIICANKEAVLSEKYQVARWGQTVWAGYSDPINAADFDSFEAFMTTVQSTWDTLWKEVYGADPTGLPDLEMDDPVLDTPAHLRYAVSASVVLVDVVFFLVLWFGARFAIWATSPLGPLRGYLLFAAILTVVASFVVYSEPFNSVAYNARKHQRRAAADPNRVRATGASISDKPAGVAPPTDLVAHSVKDVTGDSLLVDGELDSKKDA

SEQ ID NO: 25 - DNA, 7550 bp, synthetic construct
  source 1..7550: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaaagcttggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccggtacatgagcgtcccttcgaaggccctgtccctgccctcgtttctgttctctgtctttgtgttctactggtcgctgccgctctttgcgattatctaccgtatccgcttcgccaacgtcggcaagcgcaacgacatgctggactgggcgaaggccctggtcgcttttttccgcgtgacggtgctgaagcagggcgagatgtccctctaccgtggaggccgctgcctgtacctctgtaaccatcgctcctgggccgactttttcattgatacctacctcaccgagggccgcgcggccctgatgtcccgatggatggtctttttcgcctttccggtgttctgcacctctgtcctcatcctgcgcggcatcgtcctgttcaagcgaggccatatcgcggacaaggagacggcgtcgctgcaacccattgaggaggctttcaacgtctggctcgacgcgaccctccgcaagtccaccgtgccgggcctcctggtctacccggagggccatcggtccctcaagccccgctccctgccgctgaagcgcggaatgctgcacttcgctttctctcgccacctggctgtccaactgattatcacgcgaggtaaggaggaggtgatcaagccgacggatttcgactccttcgacgcgtttttccaggaggtgcagacgacctgggacgcctgctggcagctcagctaccaccagcccacggagggcgtgccgcgcctgagcctcaaggatgcgcacgagtacgactacccgccctcgatgtggtggctgcaactgggcgtcaccctcctgtcgattgtgctcctggcggccgtgctgtacggctcctggctggtgctgcgcgctggcatcctgtcgctgggatctagcggcacccaacagacgcatcgctacgtgagcgacgtccctgacctcgatcaactgaccgccctccaggagttcatgggcctgccgggcgacgtggacctccaggactacgctgacgtccccgagcggcgcatgacgctggtcctggacctggtggtcggccggacggaggagagcgcgaagggctgttgccagaagaagcgcgcggtctttgagatctcccgctaccgcgactacgcggccctgtgcacggagggctcgcaggtgatgcacgccctggacggcgtgatcacggacctgagcaacgggacctgcgactcgacggagtctgtggagcgtatcagcgccctgcccgctgagggcgtcggggagacgggtgtggagggcgcgcgacggctgctcaactacattagctttttcggcaccccgctcggcgagggcgcgtggcaggcctgccgcacggtgatggaccgacgcgctcgcgctgcctccatcctcgacgagcccttcaccctggaggaggtggctcgcgcggtgggcgggctcccgaacggcaaggcgcacggcctggaggtggccccgagcgagtgctaccgctacgcccgcctgccccgtgtggacgagcagggccgccagggcaccgaggtcaactgcatcgtcccggtcctgcatgtgctgctcgcccatatccgcaccacgggcgacttcccccaacagtttacgaagacggtggtcagccctattctgaagaagggcgacccgctggtcgcgggcaactactggggaatcgccgtcggtggcgccttcgccaagtgttacgcgtcggtgattctcaaccgactcgtccgcgcgggcgagacgctggacctccggcacccctgccaagcgggcttccgcctcgggtttggaaccgcgcatcacctgttcgtgaagcagcacctggtgcggcgacgtcgctcgggcgggtgccccgccctgcgctgtcggcgcctgccccgccggtgacacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtatcgatagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 26 - DNA, 6740 bp, synthetic construct
  source 1..6740: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaaagcttggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccggtacatgctgtgcctgtaccgttcggccgtcatctttattctctctagctccgtgctggcgttcatctgccactttctgggcgcgctcggtgcgtgcgtgctctggggtcaactgagcctctcgtctcgcaacctcttcgggctccgctgcaactgctgtctgctcaccgacagcatggaggccagctcgaaggtcctgacgttccccgcgttcctctgctccgtgttcgtgttctactggtcgctgcctatctttgcgatcatttaccgcatccggtttgcgaacctgggaaagcgcaacgatatgctggagtgggcgaagtcgctggtccggttctttggcgtgacggtcctgaaggagggcgagtcttccctgtaccgcggagggaagtgcctgtacctgtgcaaccaccgtagctgggcggacttctttatcgacgcctacctgacggaggggcgggcggccctgatgtcgcgctggctggtgtacttcgtgttccccgtgttttgtacgtcgtgcctgatcctgcggggcatcgtgctgttcaagcggggcaccatcgctgacaaggaggccttcaactcttggctcgatgccacgctgggtcggtcccatgtgccgggcctcctggtctacccggagggccaccgttccctgaagcctgcgtcgctgccgctgaagcgcggcatgctgcactacgcgtttagccgcaagatccctgtccagatcattatcacgcgaggtaaggaggaggtcctgtccgagaagcggatggccgtccgcttcggccggacgctggtgacgcagttctccaaggtcatcgagccgggccaattcgccagcttcgacgcgtttttctcggccgtgcagagcacgtgggacgagtgctggcgcggcgcctacggcgcgagcacgacctcgacgccccgccataccctgtcccacccacccacggagtacgactacccggcccgcatgcgcctgcaacaggccgcgatcacgctgttcagcatggtcgtgctggctggcgtgctggcgggctcgtggcgggcgggcgccgcggtctagcacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtatcgatagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 27 - DNA, 6653 bp, synthetic construct
  source 1..6653: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaaagcttggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccggtacatggcggtgagcaagctcctgggcctccccgcctttctgttcagcgtgttcgcgttctactggtccctccccatgctggcgatcacgtatcggctgcgcttcctgaacctcggcaagcgcaacgacatgctggactggggccgcggactcgtcaactttttcgctgtcaccatttacaaggtgggcgacggctcgctgtacaagggtggctcctgcctgtacctctgcaaccaccgctcttgggcggactttttcatcgacatctacatcacggaggggcgcggcgccatgatgagccgctggctcgtgtttttcgtgttccctatcttttgctccgccgtggtcaccctgaagggcattatcctgtttaagcggggcaccatcgccgacaaggagaagtttaacgcctggctggacgcgacgctggcttcgagcccggtgccgggcatgctggtgtacccggagggtcaccgttcgatcaagccccactcgctccccctgaagcgcggcatgctgcattacgcgttcagccgcaagctgcccgtccaggtgatcattacccgtggcaaggatgacgtgctctcggagaagaccatggcggtgcattggcgacggcaactggtgaccagctacggcaaggtgattaagccgagcgagtgcgcctcgttcgacgccttcatgcatgaggtgcagacggagtgggacaagcactgggcggaggcctacaacgcgcccaagcccccaagcaagagcctgcctcgcctcgtcccggccccgcatggctacgactacccactgagcatgcgcctgggtatgggttccgtgaccacgttctcgatcttcgccttcgcgggcgtcctgtacggcacctactgcacggctctgtgggtgctcgcgtttacgggcgcggcccagaaggtgatcgtgacggtgctcgccctgtggatgggtctggccctcacccgcgcggtgctgtagcacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtatcgatagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 28 - DNA, 6677 bp, synthetic construct
  source 1..6677: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaaagcttggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccggtacatggggcagcctctgctccagaagttcatctccctgcctaccttcctcctggcgatcttcctcctgtactggtccctcccgatctgcgtgctggtcggcaccctgaagctcctggatttcaccttcccgcggacggatatgtacaactggtgccagtttatgaccaagctgttccggatcaagatcaagaagtacggccctaacggcctgcgcaaggacacgggccgccacatcctctacctgtgcaaccaccgctcgtggaccgactttttcctggacgcttacctgacggagggcaacggctcgctcatgtcgcgctggctggtgttcgcggtcttcccggtctttatggtggcggtgatcgtgctgcgcggagtcatcctgttcaagcggggcacggtgctcgacaaggagaagttcaacgagtggctggactccaaggtgtcctcttcgccgatccccggcatgctcgtctaccccgaggggcaccgctcgctcgctcataagagcctcccgctcaagcgcgggatgctgtactacgcgcactcccgcaagtttcccgtgcagatcattatcagccgcaacaaggaggaggtgctgtccgagaagacgatgaaggcccactttggcgccacgatcgtcacgggcttctccgagctgatcgagtcggagggcaaggacatcaaggagttcctgagcgaggtccagacggcctgggacaagatgtgggactctgtgtactccgccgacgctgccgcgctccccgagctggagcatacggacgtccccaagttcgactactcgtttagcatgcgcgtgatgcagctcctggtcgtgtccggcaccgtggtcgtgttcctcggcacgatggcggccatggtcaagtgcgtgtgcgtgtgcctcgccgctacgggcgacctcggctgcaaggtgctcgtcagctcgctcgtgggctggtttctggtgtccatcctgaacagccgagcgccctccggcaagtgacacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtatcgatagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 29 - DNA, 6752 bp, synthetic construct
  source 1..6752: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaaagcttggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccggtacatgctgctcatcctgaagctgcccgcgttcctgctcgccgtgtacgtgatgtactggtcgctcccgctggccaacctgctcctcttcatcaaggcttggaaggtcgtgggcaaccgaaacgacatgtatcggtggtgccagttcctcgtccgcgcctgtgggatcaaggtgcggaagtgcggcaagcaggaactctaccgtgggggcgcgtgcctgtacctggccaaccaccgatcgtgggccgacttctttgtcgacgcctacctgacggagggccggggccaactcatgtcccgcgccgcggtgatcttcgtctttcccgtcgtgatcctgcccctgacgctggtgcgcgggtgcatttgctttcgccgggacggcgtgaaggacaaggagaagttcaaccgctggatcgacgcttgcatcggcgcctcgcccgtccccggcatctgcctgttccccgagggccaccgctcgcgcctcccccattccctcccgctgaagcgtggcatgctgaactacgcgtacagccgcaagatgccagtgcagatcgtcatctctgcgcataaggagctggccatggacgaaaagagcttccacgtccgcttcggcgccacctgcgtgaccgggtacggccccctgatgcgaaccgcggactacgcctccttcgacgagttctacaccgccctccagggcgagtggcagaagctctgggtggaggtcttctccgccgacccggcggccctcccggagctggtcatcagcgccccggcgggcttcacctaccccctgcaactgaagctgtcccagctcctgctcaccctcctggagctggcgctgatggcttggggcctcgcgtggtccgtccgtgcttggggcggggtggcggcccgcctggcgggcaccctccaactgggcgtctcgggcctccaagtcctccagtacctgcccctggtctggctggtggtcagcctggcgtcgtgctacgacagcacgctgccgcagcccgtcctggcggccgacggcacgccgctcgtcggctcctctcaagccacccgcgactccaagaaggctctgtgacacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtatcgatagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 30 - DNA, 6956 bp, synthetic construct
  source 1..6956: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaaagcttggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccggtacatgcgcctgggacgcgccatcgctcgggcgtgccgctggcccacgttcctcggctcgatcctgctcctgtactggagcgtggtctgcgtggcgctcagctaccacttcaagtggctgaacctgcgcggccgacgcaacgatgtcctgggctggagcggaggcatgaagtggtgggtccgtgccaaggtcctgaaggtcggggaccgggaactctaccgcggcccttgcgtgtacctgttcaaccaccgctcctggggcgacttcatcgtcgaccagtacgccaccgagggccgttcgctgttcatgtctcggctgaccgtcctgttcgtctttccgacgtttatgggcgctctgaaggtgctgaagtcggtgatcacctttaagcgcggcgccattgccgacaaggagcgatttaacaactggattgatgagcagcgcgcggcctctccccagacgggcctctccgtctaccccgagggccatcgctctacggccccggtgtccctgcccctgaagcgcggcatgctgtactacgcgcactcgcggcgcctgcccgtccaggtcatcattggggccaacaaggaggcgattatcagcgagaagcactgcaccgcccgcctgaaccagaccgcggccgtggggtacagcgaccctatcatgaccgccgactaccccgactttgaggcgttctgggagcaggtgcaggaggcctggaacaagacgtgggcggaggtgtacggtgctgactgggagggactgccagaactcgctccggggtccgcgggctggcaggaccccgacacgcactaccctctgtccgtgacgctgcccatggtcggggtggtcctggccaactttgcggccttcgctggcgtctcctggctggccttccgcgcgctcagcgccctcttcgccctgttcggccctgcgcagcccctggcgctggcgctggtcgcggcttacctcgccctctccttctacctgtacagccgtccggtcgacgccctccagctccacgagcgcctgcgccgtgagtccaagcatccgagccatctggcccgctccgctgcccacctggcgggcccgcgcggtgtggacgcgaacggcgctgccgctgccgaggcccgagtggcggctctgcggcacgcgcggccgacgcatcgacccacgccctacctcgggcggcgacggccgctcgcggccccacccgctcgggctccgccccggctgtcgctccgcgctccctccgcggctaccgccgtggcccgcggcatgctgtgacacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtatcgatagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 31 - DNA, 6767 bp, synthetic construct
  source 1..6767: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaaagcttggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccggtacatgtccgtgccccaactggtggcgaaggcctgtcgttggcccaccttcctcggctccattatcctcctgtactggtccgtggccttcatcaccctgagcggccacctgaagtggtggcgactccgtggtcgtcgcaacgacgtcctggcctggtcggctgggatgaagtggtgggtgcgatgccgagtcctgaaggtgggctctacggacctctaccgccagccgtgcgtgtacctgttcaaccaccgctcctggggcgacttcatcgtggaccagtacgtgaccgaggggcggtctctgttcatgtcccggctggcggtcctgggggtgttccccacgttcatctacagcctcgtgatcctcaagtccattatcctcttcaagcggggctccatcgccgataagcagcggttcaacgcctggatcgacaagcagcgagagggctcgcctcagacgggcctggcggtgtacccagagggccatcggtcgaccctgggggagcccctcccgctgaagcgcggcatgctgcattacgcgtactcccgaaagctccccgtccagatcgtcatcggtgccaacaaggaggcgattatcgcggagaagcgctgcaccgctcgcctgggccaaaccgcggtcgtgggctactcggagccgattatcaccgccgattacccggacttcgaggcgttctgggcggaggtgcaagaggcgtgggacgctcagtggcaagctgtgtttggtgccgactggcagggcctgccggaactcgccgtccaggagcccgtgacgcactaccccctgtccatcaccctgccgatgctggggatgacctggctgaacctcggcctgtttgcgtggacctgctggctgacctttcgcgccgtgcggtgggcctttgctctgtttggccccgcccagccgctggcgctggccgctgccgcggcctacctcgccctctccttctggctctactcccagccagtcaacgcgctgaaggccatgcagacccagcgcggacaccccggcaagctcgctgagagcgaggctaacctggccgcgctcgatggctcgggcgctcggcgcgacagctgacacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtatcgatagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 32 - DNA, 6749 bp, synthetic construct
  source 1..6749: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaaagcttggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccggtacatgaccctggcccgactgattgcgaaggccaccaagtggccgacgttcctgggtagcatcctcctgctctactggtccgtggtcttcgtcgcgctctcctaccacattaagtggctgaagctgaagggtcggcgcaacgacgtgctgggctgggccgctggcatgaagtggtggattcgctgtcgcgtcctgaaggtcggcacccaagagctgtaccgcgggcagtgcgtctacctcttcaaccaccgctcctggggcgacttcatcgtggaccaatacgtcaccgagggacgcagcctgttcatgagccgcctggccgtcctcggggtctttcccaccttcgtgtcggctctcgtggtcctcaagagcattatcctgttcaagcgcggctccgtggccgacaaggagaagttcaatcggtggatcgacgagcagcgcgcggccagcccacagccggggctgtcggtgtacccagagggccaccgctccaccctgggggagccgctgcccctgaagcgcggcatgctgcactacgccttctcccgcaagctccctattgtgatcggcgctaacaaggaggcgattatctccgagaagcactgcacggcccggctgaaccagacggcggtggtcggatactctgcgccgatcatgaccgccaactaccccagcttcgacgcgttctgggaggaggtgcaggaggcgtggaacgcccagtgggccgcggtcttcggggccgactgggacggcctgcccgagctggccccacaggagccggataccgagtacccggcctcgatcaccctcgggatgctgggtgtggtcgtggccaacatggccctgttctgtctcacctgctggctgagcttccggttctggcgttggctgttcgccctgttcggacccgcgcagcccgtggccctggccgctgccgcgggctacctggccctggctttttggctgtacagccgcccggtgaacgccctccaactgcacgcggagaagcgccgaccgggccaactggcccagtcggctaccaacgtcgccgcgctggacggcaagaaggcgtcctgacacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtatcgatagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 33 - DNA, 6818 bp, synthetic construct
  source 1..6818: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaaagcttggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccggtacatgctcctgctccgcctgcccgccttctggctggctgtgtggctgatgtactggagcctccctctgggcgtgctgacgctgtacatccccgtgtggcagctctggggcgcccgcaacgacacgttttactgggtcaagttcctggtgaccgccctgggcatcaagttccgcaagcgaggctcgggcgagctgtactctggccaagccccgtgcatctacctggcgaaccaccgctcgtgggcggacttctttgtggacgcgtacctcgctggcgggcgcgcgcagatgctgagccggatggcggtgctgtacgcgttcccgatgttcatggcccccgtgatcgccatgcggggctgcattgtctttaagcgcggacgcattgatgacaaggaggcgttcaacaagtggatcgacggcctcgtggaggcgtcgccgcagaacggtctgatggtctaccctgagggccaccgctctacgctggcccactcgctgcccctgaagcgcggcatgctgcactacgcgtactcccgcaagatgcccgtgcagattatcatgagcgcgaacaaggagagcgtcctctcggagaaggagctgcacgtgggcttcggttgcacggtcgtgacgtcttactcgggcctcatctcccccggcgactacgctggcttcgagggcttcatggctgcggtgcaggccgcttgggacaaggagtggcgcgacacgatgtcggcggacgcggccgggctgccgcccctgcacgtcagcgggaccattgatgacaaccactacccaccccgcctggtggtctcgcaactgtttttcagcctcctggggatcaccctcctgttcgccgcttcttcgattacctggggctggattctgcgagccaagggcgtcctgggcccccagggtgcgaagatggtggtctcctttctggtcgcgtggttcgcggccagcctggcctactgctacaacggccaactgccgaagccccgcctcctgcccgatggctccgaggccaacggcgggggtgctggctggaaggctgggggccgtccgggcggtggggccgctgccaagggaggcgcgctgaaggccgctgccgctggcgggggcggaggcggatctcccaagaagtccagctagcacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtatcgatagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 34 - DNA, 6794 bp, synthetic construct
  source 1..6794: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaaagcttggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccggtacatgtccatcccgaaggccctggcgagcgccctgcgctggcccccgtttctcttttccgtgttctgcctgtactgggacgtggctatcctggtcctgacctaccggatcaagtggctggactttaagaagaagcgaaacgacgtgctcgccctgggcaagggcctgaaggacatccaccgagtgaccatcctgaaggtgggctaccgtgagctgtaccgcaagaactgcatgtacctgtgcaaccaccgtagctgggcggacttcctcgtcgatcagtacgtcacggagggccggaccctcttcgtcggccgttgggccgtcgccgcggcttttcccctggtcatgatccccctgcgggccatccggtgcgcgatcctctttaagcgcggacacatcgcggacatcgagggattcaacaagtggattgactcccagttcgctaacagcccgcaaacgggtctgggtgtgtaccccgagggccatcgttcgacccacggcgagtcgctccctctgaagcgagggatgctgaagtacgccttcagccgaaagctcccggtgcagatcgtgatcgggggcaacaaggagtcgatcctgtccgagaagcactgcacggcgcgtttctaccagaccatcgcggtcggtttctccgaggtcctgaagcctgaggagtacgatgactttgagaacttcatgcagaaggtccaggagacgtggaaccgtgagtggagcgaggtcttctcgtccaacctgaagggcctcccggtgctgccggaggtccgggagccccaactggactacccaatggatgtgcgcatgattatggcgatctgcgcggtggtcaacatcctcatgctggcgggcgtggcctggctgacctggtgggccgtggcggccgtgatggcgctcatgggccccctgcaatggccagtcgctggcctggtcgtggcctacatcgtcgcgtctttttacgtgtactcgaaggctgatgacgtgcgtcgcacccaccaaaagatgttccaacagcggaagcaggcgcccccgattgtctccgctgcgggcgctggcgagggcgccggggagggcgccggagaggagaacggcgacaagaaggagcagtagcacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtatcgatagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 35 - DNA, 6833 bp, synthetic construct
  source 1..6833: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaaagcttggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccggtacatgtctctccagcgcacgctggactccgcgctgcgctggcccgccttcctcctgagcctgtttatcctgttttggtccgtccccttctgctcgctgacctactacatccgtttcctggacctcaagaccgagcgccggggagatgtgtttagctgggccgtcggcatgaagcggtggttcggcatcaagatcctgaaggtgggcgaccgcgaactcttccgcaaggactgcatgtacctgatcaaccatcggtcgtggggcgacttcctggtcgaccagtacctcacggagggccgatcgacctttctggcgcgcgcgatggtggcggtggcgttcccgttctttatgacggtcatcatggtgatgaaggccaccatggtctttaagcgcggcaacatcccagacaaggagaagttcaaccgctggatcgactccgagtttgcggccacgccccagaccgccatcgctgtgtacccggagggccatcggtcgaccatgaccgactcgctccccctgaagcgcggtatgctgcattacgcttactcccgcaagctcccggtccaggtgattatcgcggccaacaaggaggccgtgctgagcgagaagcacagcaccgcccgcttcgggcagacgatctgggcgggctactccgagcccatgtttcccgagaagtacgagaccttcgacaagttcatggacaaggtccaggccacgtggaacgaggagtggaacaccgtctttggcaccaagccggagggtctgcgcgagctggtctgcgaggacccgaagctcctgacgcccctgcacgtgcgtcgaggtgtctccacgatcattgcggtggtcgacatctgctttctggcgatcatgtactacagcctgcggatggcccgccgggtgtccgcggccctggggccgctggaggcgcccctcctggccctgctcgcggtctacgtgaccgcgagcctcgtgcgctactctcgcccctacgacggcgtggccgcgtctgctcgcaagggcaagctggcgcaggcgagcgccacccaagcgcccccggcgcccaagctggccgcgagcggagtggccgacgtgtccgcggccaccatcaaccgtgctgcctccctcgctgccgagccaggcgatccgcagggacgccagtgacacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtatcgatagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 36 - DNA, 6683 bp, synthetic construct
  source 1..6683: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaaagcttggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccggtacatgaaggcgatcaaggacaagatgtcgtttctgtttgcgatgctgattttttactggtcgttccccctgacggtcgcgttcggcacggactggtccctcagctacaaggcccgcaacgacatctttttctggaccgcgtttctcctgggctttttcaaggtccggacgtaccgcatcggcgagcaggggtgctaccgtgacgggagctgcatctggctgtgcaaccaccgcgactggggcgactttttcatcgacctgtacgtcgtggagggccgtgcgtgctttctgtcccgcctgatggtgctgatcgcctttccagtgtttgggttctacgggctggtcacccgcaccgcggtgtttttccggcgcaacaccaagggccgcgatttcaccaagtttaaccaattcatctccggtcagatccagcgcagcccgtgccagggcatcgtcgtgtacccggagggcacccgcaacatcaagcccgagtcgctgcccctgcgtcgcggcatgctgcgctacgcgtactccgagcgcaccccggtccagatcgtgatcaccacgaacaaggagcatgtgatctctcagaagatgcagcgagcgtccttcgggaccaagtgcatctcgggctttagcgaggtcatccggccgggcgactactccaacttcgaggacttctttgacgagatcaacaagacctggaaggccgagtggaagcgcacctacggcgcgagcatctctaacgccgtggaggtgacgccttccgagatgaagctgtacatccccttctacaacattaagctcggccagtacctgagcatgatggcctactcctacttcacggtcgccctgatggtgggagtggcctcgttcagctttgtgctgacccagtccatgctgggcctgcgcgtggtccacttcggcgggttctccaccgactgcatcgtggtcagcatcttcctgggcgtcttcgtggcctgcaccaagctcatcacccgaaagcaggactagcacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtatcgatagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 37 - DNA, 6521 bp, synthetic construct
  source 1..6521: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaaagcttggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccatggacctgtggcctggagcgtggatgctgctcctgctcctgttcctcctgctcctgttcctcctgcccacgctctggttctgctccccgtcggctaagtacttctttaagatggccttctacaacggctggattctgttcctcgccgtcctggcgatccctgtgtgtgccgtgcggggccgcaacgtggagaacatgaagatcctgcgcctcatgctcctgcatatcaagtacctctacggcatccgtgtggaggtccgcggggcgcatcacttcccgccctcccagccctacgtcgtggtctccaaccaccagtcgagcctggacctcctggggatgatggaggtcctgccgggacgctgcgtccctattgcgaagcgcgaactcctgtgggcgggctcggctggcctggcttgttggctggcgggcgtgatctttatcgaccgcaagcgcacgggcgacgccattagcgtgatgtccgaggtggcccagacgctcctgacgcaggacgtccgggtgtgggtcttcccggagggcacccgcaaccacaacgggagcatgctgcccttcaagcgcggcgcgttccacctggcggtgcaggctcaggtccccatcgtcccaatcgtgatgtcctcgtaccaggacttctactgcaagaaggagcgacgctttacctctggccaatgccaggtgcgagtcctcccgcccgtgccgacggagggcctcacccccgatgacgtcccggcgctggccgaccgagtccgacactccatgctgaccgtgttccgggagatcagcaccgatggccgcggcggaggcgactacctgaagaagccgggcggcggcggctagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtatcgatagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 38 - DNA, 6431 bp, synthetic construct
  source 1..6431: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaaagcttggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccatgtccatccctacgctgctctcgatcccctacttcgccgcgtgcgtctttgtcttctactggtctaacaccctgggctgcctgtgctacaagatcaagttcatgaacctgatcggcccccgcaaggacatgcatgactggtcggagtttatgaactggtttttcggcgtccagtatcggcaggtcggggagcagtcgctgcacaagggtgagcgctgcctgtacctgtccaaccatcggtcctgggcggactttttcgtcgacatctacctgacggagggcatggcggcccccatgagccggaacctggtgttctacgtctttccatttttcatgacctcggtgtccatcctcaagggcattatcctctttaagcgtggggtcatcgaggacaagcagaagtttaacgcgtggctcgactcgaagctcgactcttccatccgcccctccctcctggtctaccctgagggccaccgcagcacccgcgcgtcgagcctccccctcaagcgcggcatgctgcactacgcgcactcccgcaagctccctatccagattatcatgacgaagggcaaggagggtgtgctcagcgagaagcacatgcgcgcctcttacggcgtgaccctggcgacgggattctcggaggtgatcaagtcgtccaacttccctgagttcgaggcgttcgccctgaaggtgcaacaggtctgggacgcgaagtggcaggaggtctacaccgccgacatctctcgacggcctgactgctgaacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtatcgatagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 39 - DNA, 6692 bp, synthetic construct
  source 1..6692: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaaagcttggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccatgaacctggcgttcccgctggcggtggccgtgatgtactggtccctcccgatcggagccctcctgtaccgcatccgctttctcaacgtgttcggcccccgtaacgatatgctggagtgggcggagatgatgcgtgccttcctcggagtgcgcttcaagctcgtgggccaacgtcgcctgcacaagggcgagcgtatggtgtacctcgccaaccaccgctcgtgggccgactttttcctggacgtgtggacgaccgaggcgcgctgctcgatcatgtcccgctggatggtcttctttgcctttccggtgtttatggccagcgtcatcgtcctccgcggcatcgtgctgttcaagcggggcaagatcaaggacaaggacgcgtttaacgcctggctggacaagaccctggaccgccaggtcaactccggctctctgctcctgtacccggaggggacgcgtaacacgaagcctcatgcgctgcctctgaagcgcgggatgctgaagtactcctggagccgcggcctgccattccaaattatcgtcacgaccaacaaggagcgcgtcctgtcggagaagaagatggcggtcaagttcggggtgacgtgctgtacggggttttccgaggtcatcgagtccaagcgctacaaggatgacttcgacggctttatggaggaggtccagcgcgtgtgggataaggagtgggcggaggtgtacaaggccgctgcggagcccgagaagctggaggacctcacgatccccagcccggacgggttcgactacctcccctcgatgaagctgctccaggtgctggtgaccctcctgaccatcgcggtgttcagcgcgatggtcgtgggctccgcctacgccctgggtctggtggtccccgcggcccaggacgcctggaagtacgcgagctacgtcctggcgtcgatgcccatcctgggcctcgccagcaagctgctcgggggcagcaaggtcggcgctatcaagtatccgggcctcgccagcaagaagattgactgaacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtatcgatagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 40 - protein, 624 aa, Volvox carteri
MSVPSKALSLPSFLFSVFVFYWSLPLFAIIYRIRFANVGKRNDMLDWAKALVAFFRVTVLKQGEMSLYRGGRCLYLCNHRSWADFFIDTYLTEGRAALMSRWMVFFAFPVFCTSVLILRGIVLFKRGHIADKETASLQPIEEAFNVWLDATLRKSTVPGLLVYPEGHRSLKPRSLPLKRGMLHFAFSRHLAVQLIITRGKEEVIKPTDFDSFDAFFQEVQTTWDACWQLSYHQPTEGVPRLSLKDAHEYDYPPSMWWLQLGVTLLSIVLLAAVLYGSWLVLRAGILSLGSSGTQQTHRYVSDVPDLDQLTALQEFMGLPGDVDLQDYADVPERRMTLVLDLVVGRTEESAKGCCQKKRAVFEISRYRDYAALCTEGSQVMHALDGVITDLSNGTCDSTESVERISALPAEGVGETGVEGARRLLNYISFFGTPLGEGAWQACRTVMDRRARAASILDEPFTLEEVARAVGGLPNGKAHGLEVAPSECYRYARLPRVDEQGRQGTEVNCIVPVLHVLLAHIRTTGDFPQQFTKTVVSPILKKGDPLVAGNYWGIAVGGAFAKCYASVILNRLVRAGETLDLRHPCQAGFRLGFGTAHHLFVKQHLVRRRRSGGCPALRCRRLPRR

SEQ ID NO: 41 - protein, 354 aa, Astrephomene gubernaculifera
MLCLYRSAVIFILSSSVLAFICHFLGALGACVLWGQLSLSSRNLFGLRCNCCLLTDSMEASSKVLTFPAFLCSVFVFYWSLPIFAIIYRIRFANLGKRNDMLEWAKSLVRFFGVTVLKEGESSLYRGGKCLYLCNHRSWADFFIDAYLTEGRAALMSRWLVYFVFPVFCTSCLILRGIVLFKRGTIADKEAFNSWLDATLGRSHVPGLLVYPEGHRSLKPASLPLKRGMLHYAFSRKIPVQIIITRGKEEVLSEKRMAVRFGRTLVTQFSKVIEPGQFASFDAFFSAVQSTWDECWRGAYGASTTSTPRHTLSHPPTEYDYPARMRLQQAAITLFSMVVLAGVLAGSWRAGAAV

SEQ ID NO: 42 - protein, 325 aa, Edaphochlamys debaryana
MAVSKLLGLPAFLFSVFAFYWSLPMLAITYRLRFLNLGKRNDMLDWGRGLVNFFAVTIYKVGDGSLYKGGSCLYLCNHRSWADFFIDIYITEGRGAMMSRWLVFFVFPIFCSAVVTLKGIILFKRGTIADKEKFNAWLDATLASSPVPGMLVYPEGHRSIKPHSLPLKRGMLHYAFSRKLPVQVIITRGKDDVLSEKTMAVHWRRQLVTSYGKVIKPSECASFDAFMHEVQTEWDKHWAEAYNAPKPPSKSLPRLVPAPHGYDYPLSMRLGMGSVTTFSIFAFAGVLYGTYCTALWVLAFTGAAQKVIVTVLALWMGLALTRAVL

SEQ ID NO: 43 - protein, 333 aa, Dunaliella salina
MGQPLLQKFISLPTFLLAIFLLYWSLPICVLVGTLKLLDFTFPRTDMYNWCQFMTKLFRIKIKKYGPNGLRKDTGRHILYLCNHRSWTDFFLDAYLTEGNGSLMSRWLVFAVFPVFMVAVIVLRGVILFKRGTVLDKEKFNEWLDSKVSSSPIPGMLVYPEGHRSLAHKSLPLKRGMLYYAHSRKFPVQIIISRNKEEVLSEKTMKAHFGATIVTGFSELIESEGKDIKEFLSEVQTAWDKMWDSVYSADAAALPELEHTDVPKFDYSFSMRVMQLLVVSGTVVVFLGTMAAMVKCVCVCLAATGDLGCKVLVSSLVGWFLVSILNSRAPSGK

SEQ ID NO: 44 - protein, 358 aa, Scenedesmus sp.
MLLILKLPAFLLAVYVMYWSLPLANLLLFIKAWKVVGNRNDMYRWCQFLVRACGIKVRKCGKQELYRGGACLYLANHRSWADFFVDAYLTEGRGQLMSRAAVIFVFPVVILPLTLVRGCICFRRDGVKDKEKFNRWIDACIGASPVPGICLFPEGHRSRLPHSLPLKRGMLNYAYSRKMPVQIVISAHKELAMDEKSFHVRFGATCVTGYGPLMRTADYASFDEFYTALQGEWQKLWVEVFSADPAALPELVISAPAGFTYPLQLKLSQLLLTLLELALMAWGLAWSVRAWGGVAARLAGTLQLGVSGLQVLQYLPLVWLVVSLASCYDSTLPQPVLAADGTPLVGSSQATRDSKKAL

SEQ ID NO: 45 - protein, 426 aa, Micractinium conductrix
MRLGRAIARACRWPTFLGSILLLYWSVVCVALSYHFKWLNLRGRRNDVLGWSGGMKWWVRAKVLKVGDRELYRGPCVYLFNHRSWGDFIVDQYATEGRSLFMSRLTVLFVFPTFMGALKVLKSVITFKRGAIADKERFNNWIDEQRAASPQTGLSVYPEGHRSTAPVSLPLKRGMLYYAHSRRLPVQVIIGANKEAIISEKHCTARLNQTAAVGYSDPIMTADYPDFEAFWEQVQEAWNKTWAEVYGADWEGLPELAPGSAGWQDPDTHYPLSVTLPMVGVVLANFAAFAGVSWLAFRALSALFALFGPAQPLALALVAAYLALSFYLYSRPVDALQLHERLRRESKHPSHLARSAAHLAGPRGVDANGAAAAEARVAALRHARPTHRPTPYLGRRRPLAAPPARAPPRLSLRAPSAATAVARGML

SEQ ID NO: 46 - protein, 363 aa, Chlorella sorokiniana
MSVPQLVAKACRWPTFLGSIILLYWSVAFITLSGHLKWWRLRGRRNDVLAWSAGMKWWVRCRVLKVGSTDLYRQPCVYLFNHRSWGDFIVDQYVTEGRSLFMSRLAVLGVFPTFIYSLVILKSIILFKRGSIADKQRFNAWIDKQREGSPQTGLAVYPEGHRSTLGEPLPLKRGMLHYAYSRKLPVQIVIGANKEAIIAEKRCTARLGQTAVVGYSEPIITADYPDFEAFWAEVQEAWDAQWQAVFGADWQGLPELAVQEPVTHYPLSITLPMLGMTWLNLGLFAWTCWLTFRAVRWAFALFGPAQPLALAAAAAYLALSFWLYSQPVNALKAMQTQRGHPGKLAESEANLAALDGSGARRDS

SEQ ID NO: 47 - protein, 357 aa, Chlorella variabilis
MTLARLIAKATKWPTFLGSILLLYWSVVFVALSYHIKWLKLKGRRNDVLGWAAGMKWWIRCRVLKVGTQELYRGQCVYLFNHRSWGDFIVDQYVTEGRSLFMSRLAVLGVFPTFVSALVVLKSIILFKRGSVADKEKFNRWIDEQRAASPQPGLSVYPEGHRSTLGEPLPLKRGMLHYAFSRKLPIVIGANKEAIISEKHCTARLNQTAVVGYSAPIMTANYPSFDAFWEEVQEAWNAQWAAVFGADWDGLPELAPQEPDTEYPASITLGMLGVVVANMALFCLTCWLSFRFWRWLFALFGPAQPVALAAAAGYLALAFWLYSRPVNALQLHAEKRRPGQLAQSATNVAALDGKKAS

SEQ ID NO: 48 - protein, 380 aa, Raphidocelis subcapitata
MLLLRLPAFWLAVWLMYWSLPLGVLTLYIPVWQLWGARNDTFYWVKFLVTALGIKFRKRGSGELYSGQAPCIYLANHRSWADFFVDAYLAGGRAQMLSRMAVLYAFPMFMAPVIAMRGCIVFKRGRIDDKEAFNKWIDGLVEASPQNGLMVYPEGHRSTLAHSLPLKRGMLHYAYSRKMPVQIIMSANKESVLSEKELHVGFGCTVVTSYSGLISPGDYAGFEGFMAAVQAAWDKEWRDTMSADAAGLPPLHVSGTIDDNHYPPRLVVSQLFFSLLGITLLFAASSITWGWILRAKGVLGPQGAKMVVSFLVAWFAASLAYCYNGQLPKPRLLPDGSEANGGGAGWKAGGRPGGGAAAKGGALKAAAAGGGGGGSPKKSS

SEQ ID NO: 49 - protein, 372 aa, Chlorella desiccata
MSIPKALASALRWPPFLFSVFCLYWDVAILVLTYRIKWLDFKKKRNDVLALGKGLKDIHRVTILKVGYRELYRKNCMYLCNHRSWADFLVDQYVTEGRTLFVGRWAVAAAFPLVMIPLRAIRCAILFKRGHIADIEGFNKWIDSQFANSPQTGLGVYPEGHRSTHGESLPLKRGMLKYAFSRKLPVQIVIGGNKESILSEKHCTARFYQTIAVGFSEVLKPEEYDDFENFMQKVQETWNREWSEVFSSNLKGLPVLPEVREPQLDYPMDVRMIMAICAVVNILMLAGVAWLTWWAVAAVMALMGPLQWPVAGLVVAYIVASFYVYSKADDVRRTHQKMFQQRKQAPPIVSAAGAGEGAGEGAGEENGDKKEQ

SEQ ID NO: 50 - protein, 385 aa, Auxenochlorella protothecoides
MSLQRTLDSALRWPAFLLSLFILFWSVPFCSLTYYIRFLDLKTERRGDVFSWAVGMKRWFGIKILKVGDRELFRKDCMYLINHRSWGDFLVDQYLTEGRSTFLARAMVAVAFPFFMTVIMVMKATMVFKRGNIPDKEKFNRWIDSEFAATPQTAIAVYPEGHRSTMTDSLPLKRGMLHYAYSRKLPVQVIIAANKEAVLSEKHSTARFGQTIWAGYSEPMFPEKYETFDKFMDKVQATWNEEWNTVFGTKPEGLRELVCEDPKLLTPLHVRRGVSTIIAVVDICFLAIMYYSLRMARRVSAALGPLEAPLLALLAVYVTASLVRYSRPYDGVAASARKGKLAQASATQAPPAPKLAASGVADVSAATINRAASLAAEPGDPQGRQ

SEQ ID NO: 51 - protein, 335 aa, Chloropicon primus
MKAIKDKMSFLFAMLIFYWSFPLTVAFGTDWSLSYKARNDIFFWTAFLLGFFKVRTYRIGEQGCYRDGSCIWLCNHRDWGDFFIDLYVVEGRACFLSRLMVLIAFPVFGFYGLVTRTAVFFRRNTKGRDFTKFNQFISGQIQRSPCQGIVVYPEGTRNIKPESLPLRRGMLRYAYSERTPVQIVITTNKEHVISQKMQRASFGTKCISGFSEVIRPGDYSNFEDFFDEINKTWKAEWKRTYGASISNAVEVTPSEMKLYIPFYNIKLGQYLSMMAYSYFTVALMVGVASFSFVLTQSMLGLRVVHFGGFSTDCIVVSIFLGVFVACTKLITRKQD

SEQ ID NO: 52 - protein, 283 aa, Homo sapiens
MDLWPGAWMLLLLLFLLLLFLLPTLWFCSPSAKYFFKMAFYNGWILFLAVLAIPVCAVRGRNVENMKILRLMLLHIKYLYGIRVEVRGAHHFPPSQPYVVVSNHQSSLDLLGMMEVLPGRCVPIAKRELLWAGSAGLACWLAGVIFIDRKRTGDAISVMSEVAQTLLTQDVRVWVFPEGTRNHNGSMLPFKRGAFHLAVQAQVPIVPIVMSSYQDFYCKKERRFTSGQCQVRVLPPVPTEGLTPDDVPALADRVRHSMLTVFREISTDGRGGGDYLKKPGGGG

SEQ ID NO: 53 - protein, 253 aa, Chlamydomonas eustigma
MSIPTLLSIPYFAACVFVFYWSNTLGCLCYKIKFMNLIGPRKDMHDWSEFMNWFFGVQYRQVGEQSLHKGERCLYLSNHRSWADFFVDIYLTEGMAAPMSRNLVFYVFPFFMTSVSILKGIILFKRGVIEDKQKFNAWLDSKLDSSIRPSLLVYPEGHRSTRASSLPLKRGMLHYAHSRKLPIQIIMTKGKEGVLSEKHMRASYGVTLATGFSEVIKSSNFPEFEAFALKVQQVWDAKWQEVYTADISRRPDC

SEQ ID NO: 54 - protein, 340 aa, unidentified
MNLAFPLAVAVMYWSLPIGALLYRIRFLNVFGPRNDMLEWAEMMRAFLGVRFKLVGQRRLHKGERMVYLANHRSWADFFLDVWTTEARCSIMSRWMVFFAFPVFMASVIVLRGIVLFKRGKIKDKDAFNAWLDKTLDRQVNSGSLLLYPEGTRNTKPHALPLKRGMLKYSWSRGLPFQIIVTTNKERVLSEKKMAVKFGVTCCTGFSEVIESKRYKDDFDGFMEEVQRVWDKEWAEVYKAAAEPEKLEDLTIPSPDGFDYLPSMKLLQVLVTLLTIAVFSAMVVGSAYALGLVVPAAQDAWKYASYVLASMPILGLASKLLGGSKVGAIKYPGLASKKID

SEQ ID NO: 55 - protein, 156 aa, Volvox reticuliferus
MSVISKALSFPSFLFSVFVFYWSLPLFAIIYRIRFMNVSKRNDMLDWAKALVAFFRVTVLKQGEMTLYRGARCLYLCNHRSWADFFIDTYLTEGRAALMSRWLVYFVFPVFCTSVIILRGIVLFKRGHIANKEAFNAWLDATLAKSTVPGLLVYPE

SEQ ID NO: 56 - protein, 360 aa, Chlorella vulgaris
MPVPRIVAKATRWPTFLGSILLLFWSVVCVALSYHIKWLKLRGKRNDVLGWAAGMKWWIRCRVLKVGSQDLYREKCVYLFNHRSWGDFIVDQYVTEGRSLFMSRMAVLFVFPTFISALTVLKSIILFKRGAIADKEKFNAWIDEQRANSPQSGLSVYPEGHRSTKRDSLPLKRGMLYYAFSRKLPVQIVIGANKEAIISEKHLTARLGQTAVVGYSEPIMTAEYADFDAFWAKVQETWDRQWQTVFGAAWEGLPELEPVEPDTEYPLSITGRMVLVVIANLLLFCCACWLTFHFWTWLFALFGPLQPAALALSATYLALALWIYSQPVNALQVHAKKLRPGQPAQPAVDMAALLETKKAS

SEQ ID NO: 57 - DNA, 5697 bp, synthetic construct
  source 1..5697: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccccggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaaagcttgcctgctcaagcgggcgctcaacatgcagagcgtcagcgagacgggctgtggagatcgggagacggacgaggccgcctctgccctgtttgaactgagcgtcagcgctggctaaggggagggagactcatccccacgcccgcgccagggctctgatcccgtctcgggcggtgatcggcgcgcatgactacgacccaacgacgtacgagactgatgtcggtcccgacgaggagcgccgcgaggcactccagggctgccgaccatgtttacaccgaccaaaagcactcgctcgcatccattccgtgcgcccgcacatgcatcatcttttggtaccgatttcggtcttgttttacccctacgacctgccttccaaggtgtgagcaactcgcccggacatgaccgagggtgatcatccggatccccaggccccagcagcccctgccagaatggctcgcgctttccagcctgcaggcccgtctcccaggtcgacgcaacctacatgaccaccccaatctgtcccagaccccaaacaccctcctgccctgcttctctgtgatcgctgatcagcaacacccggggaattcatgagcgtgatctcgaaggccctctcgtttccatcctttctgttcagcgtgttcgtgttctactggtcgctgccgctgtttgcgatcatttaccgcatccgtttcatgaacgtgtcgaagcgcaacgacatgctcgactgggcgaaggcgctcgtcgcctttttccgcgtcaccgtgctgaagcagggcgagatgaccctctaccgcggcgcccgatgcctgtacctgtgcaaccaccggagctgggcggattttttcatcgacacctacctgacggagggccgggcggccctgatgtcgcgctggctcgtctactttgtcttcccggtgttctgcacctccgtcattatcctgcgtgggatcgtgctgttcaagcgtgggcatatcgccaacaaggaggccttcaacgcgtggctcgacgccacgctggccaagtccacggtgccgggcctcctggtctaccccgagtagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggccggcccgcgtctcgaacagagcgcggcggccgcatcgatacctgcaggaagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 58 - DNA, 6309 bp, synthetic construct
  source 1..6309: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccccggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaaagcttgcctgctcaagcgggcgctcaacatgcagagcgtcagcgagacgggctgtggagatcgggagacggacgaggccgcctctgccctgtttgaactgagcgtcagcgctggctaaggggagggagactcatccccacgcccgcgccagggctctgatcccgtctcgggcggtgatcggcgcgcatgactacgacccaacgacgtacgagactgatgtcggtcccgacgaggagcgccgcgaggcactccagggctgccgaccatgtttacaccgaccaaaagcactcgctcgcatccattccgtgcgcccgcacatgcatcatcttttggtaccgatttcggtcttgttttacccctacgacctgccttccaaggtgtgagcaactcgcccggacatgaccgagggtgatcatccggatccccaggccccagcagcccctgccagaatggctcgcgctttccagcctgcaggcccgtctcccaggtcgacgcaacctacatgaccaccccaatctgtcccagaccccaaacaccctcctgccctgcttctctgtgatcgctgatcagcaacacccggggaattcatgcccgtcccccggatcgtggcgaaggccacccgctggcccacgttcctcggctctattctgctcctgttttggtctgtcgtgtgcgtcgcgctcagctaccacatcaagtggctgaagctccgcggcaagcgcaacgatgtgctgggctgggccgcgggcatgaagtggtggattcgatgccgggtcctcaaggtgggctcgcaggatctgtaccgcgagaagtgcgtctacctctttaaccaccgctcctggggagacttcatcgtggaccagtacgtcacggagggccggagcctcttcatgagccgcatggccgtcctgttcgtctttcccacctttatctccgcgctgacggtgctgaagtcgattatcctgttcaagcggggtgccatcgccgacaaggagaagtttaacgcttggatcgacgagcagcgagccaactcgcctcagtcgggcctgtctgtgtacccagagggccaccgctcgacgaagcgcgactcgctgcccctcaagcgagggatgctgtactacgcgttcagccgaaagctgcccgtgcagatcgtgatcggcgccaacaaggaggctattatcagcgagaagcacctcacggcgcggctggggcagaccgccgtggtcggctacagcgagcccattatgaccgcggagtacgccgacttcgacgccttctgggcgaaggtccaggagacgtgggatcgccagtggcagacggtcttcggcgcggcctgggagggcctgcctgagctggagcctgtggagccagacacggagtaccccctctccatcacgggccgcatggtgctggtggtcattgcgaacctgctcctgttctgctgtgcttgctggctgacgtttcatttctggacgtggctgttcgccctgttcggcccgctccagcccgctgccctggccctgtctgcgacctacctggccctggctctctggatctactctcaacccgtgaacgccctccaggtgcacgcgaagaagctgcgtccgggccaacctgcccagcccgccgtggacatggcggccctcctggagaccaagaaggcgtcgtagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggccggcccgcgtctcgaacagagcgcggcggccgcatcgatacctgcaggaagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 59 - DNA, 6390 bp, synthetic construct
  source 1..6390: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggactttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaaagcttgcctgctcaagcgggcgctcaacatgcagagcgtcagcgagacgggctgtggagatcgggagacggacgaggccgcctctgccctgtttgaactgagcgtcagcgctggctaaggggagggagactcatccccacgcccgcgccagggctctgatcccgtctcgggcggtgatcggcgcgcatgactacgacccaacgacgtacgagactgatgtcggtcccgacgaggagcgccgcgaggcactccagggctgccgaccatgtttacaccgaccaaaagcactcgctcgcatccattccgtgcgcccgcacatgcatcatcttttggtaccgatttcggtcttgttttacccctacgacctgccttccaaggtgtgagcaactcgcccggacatgaccgagggtgatcatccggatccccaggccccagcagcccctgccagaatggctcgcgctttccagcctgcaggcccgtctcccaggtcgacgcaacctacatgaccaccccaatctgtcccagaccccaaacaccctcctgccctgcttctctgtgatcgctgatcagcaacacccggggaattcatgaacctggcgttcccgctggcggtggccgtgatgtactggtccctcccgatcggagccctcctgtaccgcatccgctttctcaacgtgttcggcccccgtaacgatatgctggagtgggcggagatgatgcgtgccttcctcggagtgcgcttcaagctcgtgggccaacgtcgcctgcacaagggcgagcgtatggtgtacctcgccaaccaccgctcgtgggccgactttttcctggacgtgtggacgaccgaggcgcgctgctcgatcatgtcccgctggatggtcttctttgcctttccggtgtttatggccagcgtcatcgtcctccgcggcatcgtgctgttcaagcggggcaagatcaaggacaaggacgcgtttaacgcctggctggacaagaccctggaccgccaggtcaactccggctctctgctcctgtacccggaggggacgcgtaacacgaagcctcatgcgctgcctctgaagcgcgggatgctgaagtactcctggagccgcggcctgccattccaaattatcgtcacgaccaacaaggagcgcgtcctgtcggagaagaagatggcggtcaagttcggggtgacgtgctgtacggggttttccgaggtcatcgagtccaagcgctacaaggatgacttcgacggctttatggaggaggtccagcgcgtgtgggataaggagtgggcggaggtgtacaaggccgctgcggagcccgagaagctggaggacctcacgatccccagcccggacgggttcgactacctcccctcgatgaagctgctccaggtgctggtgaccctcctgaccatcgcggtgttcagcgcgatggtcgtgggctccgcctacgccctgggtctggtggtccccgcggcccaggacgcctggaagtacgcgagctacgtcctggcgtcgatgcccatcctgggcctcgccagcaagctgctcgggggcagcaaggtcggcgctatcaagtatccgggcctcgccagcaagaagattgactgaacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccccggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 60 - DNA, 6302 bp, synthetic construct
  source 1..6302: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggactttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcattcgccgctcagcctacacgtcttctccgatacctttccctcattgcattttatgccagactgggtcccagcctgggtgggtgctcccgctcgattgctcgtgtcggaggcggggcacccccgctctctctatttatcactgcctctccccgaccaaccctgacgactgtaaccctgccagaaacaattcagcctcaacaagccgagttgtgcacaagggagactaattttttagacgggaagcaacccgcttccagaagcatccggacgggggtagcgaggctgtgtcgagcgccgtggggatctggccgatgaggtgcccgaaacccgtgtacagctcagcggctgggatcatcgacccccgggatcatcgaccccgtgggccgggcccccggaccctataactaaaagccgacgccagcgcaaaaccacaaacatttactccttaatcctcgctcctccttcatatacaaccacaagtaatcaactcaccgaattcatgaacctggcgttcccgctggcggtggccgtgatgtactggtccctcccgatcggagccctcctgtaccgcatccgctttctcaacgtgttcggcccccgtaacgatatgctggagtgggcggagatgatgcgtgccttcctcggagtgcgcttcaagctcgtgggccaacgtcgcctgcacaagggcgagcgtatggtgtacctcgccaaccaccgctcgtgggccgactttttcctggacgtgtggacgaccgaggcgcgctgctcgatcatgtcccgctggatggtcttctttgcctttccggtgtttatggccagcgtcatcgtcctccgcggcatcgtgctgttcaagcggggcaagatcaaggacaaggacgcgtttaacgcctggctggacaagaccctggaccgccaggtcaactccggctctctgctcctgtacccggaggggacgcgtaacacgaagcctcatgcgctgcctctgaagcgcgggatgctgaagtactcctggagccgcggcctgccattccaaattatcgtcacgaccaacaaggagcgcgtcctgtcggagaagaagatggcggtcaagttcggggtgacgtgctgtacggggttttccgaggtcatcgagtccaagcgctacaaggatgacttcgacggctttatggaggaggtccagcgcgtgtgggataaggagtgggcggaggtgtacaaggccgctgcggagcccgagaagctggaggacctcacgatccccagcccggacgggttcgactacctcccctcgatgaagctgctccaggtgctggtgaccctcctgaccatcgcggtgttcagcgcgatggtcgtgggctccgcctacgccctgggtctggtggtccccgcggcccaggacgcctggaagtacgcgagctacgtcctggcgtcgatgcccatcctgggcctcgccagcaagctgctcgggggcagcaaggtcggcgctatcaagtatccgggcctcgccagcaagaagattgactgacacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccccggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 61 - DNA, 6552 bp, synthetic construct
  source 1..6552: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggactttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcattctaccggggctgcatgaccaacctgctgcggaccacccccgcggccgcggtgaccttcaccagctttgagctgatcagcatggccctggcgcggtgggccgagaacgaggcgcccgcgcgcgcgcccacgcgcgggcccgcgcggccgccgctcgccgccatcgcgctcgtgaaccaggcccccggcgccgaggaggagccgggcgcggacgcggcgcacgcagccgaccgcctgcacggcatcgccgcccacacgggccggctggccgaggagccctgatattttgttcccctctcagctgaaatctgtgtctttcttgtgcctatttcatgtacacgatccagactgtgccgtcgcatgcggcgaggcagaggctggggcgcccaaggctggtcggcccattcaaaaaatgaatcgcgagatcgcatgggtgcatcgcaagaaagggcggaagatcgaacctcgtgcgcactctcgaaacaacgtcgactggagtgacataaccatgggacctcacagtcggccaccacaacgtttctgatcgctttcacaaggcgaagatagtgttcacgtcccaatcggaggctcaacgcgggtacgggcccgggtttctggcaaactgatctcccggacgcggagggccgtttggcgacctgcgattcccggggcgccctctaattgaaatttgaccgccttccacgcgtgataggtggtttacggcgcgactgagctcaaagcaaggaattcatgaacctggcgttcccgctggcggtggccgtgatgtactggtccctcccgatcggagccctcctgtaccgcatccgctttctcaacgtgttcggcccccgtaacgatatgctggagtgggcggagatgatgcgtgccttcctcggagtgcgcttcaagctcgtgggccaacgtcgcctgcacaagggcgagcgtatggtgtacctcgccaaccaccgctcgtgggccgactttttcctggacgtgtggacgaccgaggcgcgctgctcgatcatgtcccgctggatggtcttctttgcctttccggtgtttatggccagcgtcatcgtcctccgcggcatcgtgctgttcaagcggggcaagatcaaggacaaggacgcgtttaacgcctggctggacaagaccctggaccgccaggtcaactccggctctctgctcctgtacccggaggggacgcgtaacacgaagcctcatgcgctgcctctgaagcgcgggatgctgaagtactcctggagccgcggcctgccattccaaattatcgtcacgaccaacaaggagcgcgtcctgtcggagaagaagatggcggtcaagttcggggtgacgtgctgtacggggttttccgaggtcatcgagtccaagcgctacaaggatgacttcgacggctttatggaggaggtccagcgcgtgtgggataaggagtgggcggaggtgtacaaggccgctgcggagcccgagaagctggaggacctcacgatccccagcccggacgggttcgactacctcccctcgatgaagctgctccaggtgctggtgaccctcctgaccatcgcggtgttcagcgcgatggtcgtgggctccgcctacgccctgggtctggtggtccccgcggcccaggacgcctggaagtacgcgagctacgtcctggcgtcgatgcccatcctgggcctcgccagcaagctgctcgggggcagcaaggtcggcgctatcaagtatccgggcctcgccagcaagaagattgactgaacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccccggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 62 - DNA, 6592 bp, synthetic construct
  source 1..6592: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggactttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcagatgctttgaagatggacgttggtggtatggaatgcaaaattcaagtgcaagtcgacgctcgctgtttagtcgctaagaagcaacggttgtctacgaaaatgtaaacaattgaggcgtgatgcgtgaccccaaggcaggaaggctcaaggtgaaagctttctaggttacagaatttatctactcgagaatggtttgttgcagcttgaaaggtttacctaaggcggtttcgttatcatctgatctgcccatgcaaaatgtgagcgcgtcggggttgactactcgtcaggtcatgcaccgacccgcgcgcggaccatatcttcgtcggaaaagaggggatgaggggcaaaatgtcccattcttcctttccttacgtacgccacatagcatatctcgggcagatgtccgcttatcgtcagcgattggatatccaagatacggtcgcgtggtgtgttgactcatcggcgcgtcttcgcagctggcagggcgccgaccccctcccacccgcatataacctccccgcaccgaagcaaaggtcatccacaacttcaacacatccacgcattttctgtcgcgtctttagtgcctccagtctcaaggcaccttctctaccactccgaccctcccctcctcaaccggtaagccagtttgcttcgcgtattccacctactcgtgccagaaccgggcttgtaccatgttgacaaaccttgttgggggcagcaaaggttcgcggtcggccgcccgcatgcttaccgactccctgatcgccccttcgtcgcagacaccgaattcatgaacctggcgttcccgctggcggtggccgtgatgtactggtccctcccgatcggagccctcctgtaccgcatccgctttctcaacgtgttcggcccccgtaacgatatgctggagtgggcggagatgatgcgtgccttcctcggagtgcgcttcaagctcgtgggccaacgtcgcctgcacaagggcgagcgtatggtgtacctcgccaaccaccgctcgtgggccgactttttcctggacgtgtggacgaccgaggcgcgctgctcgatcatgtcccgctggatggtcttctttgcctttccggtgtttatggccagcgtcatcgtcctccgcggcatcgtgctgttcaagcggggcaagatcaaggacaaggacgcgtttaacgcctggctggacaagaccctggaccgccaggtcaactccggctctctgctcctgtacccggaggggacgcgtaacacgaagcctcatgcgctgcctctgaagcgcgggatgctgaagtactcctggagccgcggcctgccattccaaattatcgtcacgaccaacaaggagcgcgtcctgtcggagaagaagatggcggtcaagttcggggtgacgtgctgtacggggttttccgaggtcatcgagtccaagcgctacaaggatgacttcgacggctttatggaggaggtccagcgcgtgtgggataaggagtgggcggaggtgtacaaggccgctgcggagcccgagaagctggaggacctcacgatccccagcccggacgggttcgactacctcccctcgatgaagctgctccaggtgctggtgaccctcctgaccatcgcggtgttcagcgcgatggtcgtgggctccgcctacgccctgggtctggtggtccccgcggcccaggacgcctggaagtacgcgagctacgtcctggcgtcgatgcccatcctgggcctcgccagcaagctgctcgggggcagcaaggtcggcgctatcaagtatccgggcctcgccagcaagaagattgactgaacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccccggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 63 - protein, 286 aa, Ostreococcus sp.
MAPVFALSFQLSIFASTDGARVDRRARRSPGFEWGKFMTRSVANARFKLAPGSGTLAREKRRTVWLCNHRAWSDFFLDIYLTEGRAFTMSRMLVAYAFPLFMVPAMVCGVVFGFKRDAGDKEKLNAALDAHFDAFGGEFAGMVCYPEGTRNVRENSMPLKRGMLRYAHSRKMAVQCVCTARKERVFSSHLTCAERGLALPVYFAEVIHAEDYPDFEDFYNEIRSRWDNAWSAANGVSAAEAESLPDYTPREHVVRCTPRHDAVIVSSALLTVLGVALTWRACSKLL

SEQ ID NO: 64 - protein, 197 aa, Coccomyxa subellipsoidea
MGVLEKIQTLPAFFLGVFFLYWSLPLAALMNKVKFLKLAGKRNDAYGWGVALQQLFGVTMLRLPGPQLANPCGPIIFLSNHRSWADFYLDVVATEGNAQMLSRMAVALVFPMFMGAVCVIRSVILFQRGRNHEKEAFNNMIDRKMAASPVDGLIVYPEGHRSTLRHSLPLKRGMLNYAYIRKIPVQVIFCNAVLCLL

SEQ ID NO: 65 - DNA, 6259 bp, synthetic construct
  source 1..6259: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggactttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcattcgccgctcagcctacacgtcttctccgatacctttccctcattgcattttatgccagactgggtcccagcctgggtgggtgctcccgctcgattgctcgtgtcggaggcggggcacccccgctctctctatttatcactgcctctccccgaccaaccctgacgactgtaaccctgccagaaacaattcagcctcaacaagccgagttgtgcacaagggagactaattttttagacgggaagcaacccgcttccagaagcatccggacgggggtagcgaggctgtgtcgagcgccgtggggatctggccgatgaggtgcccgaaacccgtgtacagctcagcggctgggatcatcgacccccgggatcatcgaccccgtgggccgggcccccggaccctataactaaaagccgacgccagcgcaaaaccacaaacatttactccttaatcctcgctcctccttcatatacaaccacaagtaatcaactcaccaatgtcgctcattagcaaggccctctccctgcccagcttcctgctctccgtcttcgtgttctactggtcgctgccgatcttcgccatcccctaccgcatccggttcgccaacgtcggcaagcggaacgacatgctcgactgggcgaaggctctgttcgcgtttttccgcgtcaccgtcctgaagcagggggagcagggcctctaccgcggtggcccatgcctgtacctgtgcaaccaccgctcgtgggcggacttctttgtggacgtctacctgacggagggccgggccgcgctgatgtcccgctggctggtgtttttcgtcttccctgtgttctgcacctcctgcctcatcctgcggggcatcgtgctgttcaagcgcgggaccatcctcgacaaggaggcctttaacgcgtggctcgacgccacgctgcggcgctccaccgtccccggcctgctcatttaccccgaggggcaccgctcgctgaagccgacctcgctgccgctgaagcggggcatgctgcacttcgcttacagccgacggctccccgtccaactgatcgtgacccgcggcaaggaggacatcctgtcggagaagacgatggccatgaagtacggccgcaccctcgtgaccacgtactcgaaggtcctgaagcccacggactacgacagcttcgacgccttcatgggtgacgtccagacgagctgggacgcgggctggaaggcctcctacgaggcccccaccaagggggtgccccaactgtccatggcgggtgcccacgagtacgactacccggcgaccatgcggtggcagcaactggcgatcaccctcttctcgatcgtcgtgttcgcggccgtgctgtacggcacgtggtggggcctgggtgccgtcctggctgcggctggccccctcggccagcaagtggtcgtggtcctggcggccacctcctggctgggcgccagcctgatccggtcctgccgctaggtaccacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccccggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 66 - DNA, 6265 bp, synthetic construct
  source 1..6265: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggactttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcattcgccgctcagcctacacgtcttctccgatacctttccctcattgcattttatgccagactgggtcccagcctgggtgggtgctcccgctcgattgctcgtgtcggaggcggggcacccccgctctctctatttatcactgcctctccccgaccaaccctgacgactgtaaccctgccagaaacaattcagcctcaacaagccgagttgtgcacaagggagactaattttttagacgggaagcaacccgcttccagaagcatccggacgggggtagcgaggctgtgtcgagcgccgtggggatctggccgatgaggtgcccgaaacccgtgtacagctcagcggctgggatcatcgacccccgggatcatcgaccccgtgggccgggcccccggaccctataactaaaagccgacgccagcgcaaaaccacaaacatttactccttaatcctcgctcctccttcatatacaaccacaagtaatcaactcaccaatgtcgctgatcagcaaggcctcttccctgccgagcttcctgttttcggtgttcgtcttctactggtccctgccaatctttgcggtcccgtaccgtatccggtttgctaacatggggaagcgtaacgacatgctggactgggctaaggcgctggtggcctttttccgcgtcaccgtcctgaagcagggcgagcagggcctgtaccggggcgggacgtgcctctacctgtgcaaccaccgaagctgggccgactttttcatcgacgtctacctgacggagggccgtgcggccctgatgtcgcggtggctcgtgtttttcgtcttccctgtcttttgcacgagctgcctcatcctccgaggcatcgtcctgtttaagcggggcacgatcctggacaaggaggccttcaacgcctggctcgacgccacgctccgacgctcgatcgtgcctggcctggtggtctaccccgaggggcatcgctccctcaagcccacctcgctccccctgaagcgaggcatgctgcacttcgcgtttggccgtcagctcccggtgcaactgatcgtgacgcgcggaaaggaggacgtgctgtccgagaagaccatggccatgaagtacggccgcacgctggtgacgagctactctaaggtgctgaagccctctgactacgccagcttcgacgccttcatgacggacatccagacgtgctgggactcttgctggcgtgccgcttacgaggccccgaccaagggcgtcccccagctctccatggtcggcgcccacgagtacgactacccccggaccatttggtggcagcaactctggatcaccctggcctccatcctgatcttcgctggcgtgctctacggcacctggcgcggcctggcttccctcctggctgccgcgaccacgagcggcgcgctgggcccacaggtcctggtgaccgtggccgcggcctgggcgggctcctcgctcctgcggtcgctgctgtgagtaccacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccccggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 67 - DNA, 6253 bp, synthetic construct
  source 1..6253: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggactttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcattcgccgctcagcctacacgtcttctccgatacctttccctcattgcattttatgccagactgggtcccagcctgggtgggtgctcccgctcgattgctcgtgtcggaggcggggcacccccgctctctctatttatcactgcctctccccgaccaaccctgacgactgtaaccctgccagaaacaattcagcctcaacaagccgagttgtgcacaagggagactaattttttagacgggaagcaacccgcttccagaagcatccggacgggggtagcgaggctgtgtcgagcgccgtggggatctggccgatgaggtgcccgaaacccgtgtacagctcagcggctgggatcatcgacccccgggatcatcgaccccgtgggccgggcccccggaccctataactaaaagccgacgccagcgcaaaaccacaaacatttactccttaatcctcgctcctccttcatatacaaccacaagtaatcaactcaccaatgtccatcgtcaccaaggtgctgaacctcccctcgttcctgttcagcgtgttcgtcttttactggtccctgcccctgtttgcgatcctgtaccgcatccgcttcgccaacgtgggcaagcgcaacgacatgctggactgggcgaagacgctcgtcggctttttccacgtgaccatcctgaagcagggcgagctgtctctctaccgggacgggccgtgcctgtacctgtgcaaccaccgctcctgggccgacttctttatcgacacctacctgacggagggtcgcgctgccctgatgagccgctggctggtgtacttcgtgttcccggtcttctgcacgtccgtgattatcctgcgcggcatcgtgctgttcaagcgcgggtctatcgcggacaaggaggccttcaacgcgtggctggacgccacgctgcgtaagtccattgtccccgcgctcctggtgtacccggagggccaccgctcgctgcgccccacgtctctccctctgaagcgcggcatgctgcactacgcctacagccgccgactccctgtgcagctcatcgtcacgtcgggcaaggaggccgtcatgagcgagaagaccatgagcgtccgcttcggccgcaccctcgtcaccacgtactccaaggtgctgcgccccgcggactaccccacgtttgacgccttcttttccgacatccaagctacctgggacggctgctgggacgccgcttacaagcagccgtctgcgggtgcgccccagctccgcctggtggacccgcatggctacaactacccggcccgcatgtggtggctccaactgggcgtgaccctcctgtcgatcggggtgatggccgctgtgatcgccgcgagctggtggggtctgggcgctctcctggccgcgacgggcccggctaagcaggtggtcctgggcgtcgtggtctcgctgctcggcgcgagcctcctgcgatcgtgcctgtgagtaccacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccccggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 68 - DNA, 6253 bp, synthetic construct
  source 1..6253: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggactttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcattcgccgctcagcctacacgtcttctccgatacctttccctcattgcattttatgccagactgggtcccagcctgggtgggtgctcccgctcgattgctcgtgtcggaggcggggcacccccgctctctctatttatcactgcctctccccgaccaaccctgacgactgtaaccctgccagaaacaattcagcctcaacaagccgagttgtgcacaagggagactaattttttagacgggaagcaacccgcttccagaagcatccggacgggggtagcgaggctgtgtcgagcgccgtggggatctggccgatgaggtgcccgaaacccgtgtacagctcagcggctgggatcatcgacccccgggatcatcgaccccgtgggccgggcccccggaccctataactaaaagccgacgccagcgcaaaaccacaaacatttactccttaatcctcgctcctccttcatatacaaccacaagtaatcaactcaccaatgtccgtgatctacaaggctctgtcgctgccatccttcctgttctctgtgttcgtgttctactggtctctgcccctgttcgcgtctgtctaccgcatccggtttgcgaacttcggcaagcgcaacgacatgctggactgggcgaaggccctcgtggccttctttcgcgtcaccgtgctcaagcaaggcgagatgtcgctctaccgcggaggcccttgtctctacctgtgcaaccatcggtcgtgggctgacttctttatcgacacgtacctgaccgagggccgcgcggccctgatgagccgctggctggtctacttcgtgtttccggtgttttgtaccagcgtgctgatcctgcggggcatcgtcctgtttaagcggggtcacatcgtggacaaggaggcgttcaacgcgtggctcgacgccaccctgttcaagtctacggtgccgggcctcctggtgtaccccgagggccaccgctccctcaagcccgcctccctgcccctgaagcgtggcatgctccactttgctttttcccgcaagctgcccgtccaggtcatttgcacccgcggcaaggaggaggtcctgtgcgagaagacgctgtccgcgcggtggggccgcacgctggtggtcaccttctccaaggtgatcaagccgtccgagtacgactcgttcgacgcgtttttcaacgacgtgcaatctacctgggacgcgtgctggcagctctcctaccaccagccaacggcgggcgtcccggagctgtccatgaaggacgcccacgagtacgactacccccgctctatgtggtggctgcaactgggtatcaccctcctgtcctttattatcctggccgcggtgctgtggggctcttgggtcgctgtgcgcgcgggtttcgccacgctcggcagcgtccaacaggccgtggtcctgggcaccgcggggtggatcggcgcctcgatggtgtgcggcttcctctgagtaccacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccccggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 69 - DNA, 6256 bp, synthetic construct
  source 1..6256: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggactttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcattcgccgctcagcctacacgtcttctccgatacctttccctcattgcattttatgccagactgggtcccagcctgggtgggtgctcccgctcgattgctcgtgtcggaggcggggcacccccgctctctctatttatcactgcctctccccgaccaaccctgacgactgtaaccctgccagaaacaattcagcctcaacaagccgagttgtgcacaagggagactaattttttagacgggaagcaacccgcttccagaagcatccggacgggggtagcgaggctgtgtcgagcgccgtggggatctggccgatgaggtgcccgaaacccgtgtacagctcagcggctgggatcatcgacccccgggatcatcgaccccgtgggccgggcccccggaccctataactaaaagccgacgccagcgcaaaaccacaaacatttactccttaatcctcgctcctccttcatatacaaccacaagtaatcaactcaccaatgagcgcgctgaagaaggccctgtccctgcccagcttcctgttttctgtcttcgtcttctactggagcctgccaatcttcgcctcggtgtaccgcatccgcttcgcgcgcgctggaaagcgcaacgacatgctggactgggctacctccctggtggcgttctttcgcgtgaccgtgctcaagcagggtcagttttcgatctaccgcggaggcccctgcatctacctgtgcaaccaccgtagctgggccgatttctttatcgacatgtacctcacggagggccgcgccgcgttcatgtcccgttggatggtcttctttgtcttccccgtcttctgctccgccgtgctgattctgaagggcattatcctgtttaagcggggccgcatcctggacaaggaggcgtttaacgcctggctggacgccaccctggcgaagtcgacgatccccggcctggtcgtgtacccggagggccatcgctctctgaagccgacgtcgctgcccctgaagcgcgggatgctgcactacgcgttttcccgtaagctcccagtgcaactgatcgtcacccgcggcaaggaggaggtgctgtcggagaagaccctgtccatgcgcttcgggcggaccctggtgacctcttactcgaaggtgatgaagcccagcgactacggctccttcgatgcctttatgaccgacgtgcagtctggctgggaccagtgctggaaggaggcctactccgcgccgacccacggcctgccgcagctctcgatggctggcgccaccgagtacgactaccccgcttccatgcgctggcagcacctggcgatgacgctgttttcgatcgtcgtgtttgcgggtgtcctggccgcgagctggtggggcgtcggcgccctcctgtctgctgcgggccctacgggcaagcaggtggtcgtgctcctggcctcgtgctggttcggcgccagcctcctgcgtagcttcctgtgagtaccacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccccggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 70 - DNA, 6256 bp, synthetic construct
  source 1..6256: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggactttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcattcgccgctcagcctacacgtcttctccgatacctttccctcattgcattttatgccagactgggtcccagcctgggtgggtgctcccgctcgattgctcgtgtcggaggcggggcacccccgctctctctatttatcactgcctctccccgaccaaccctgacgactgtaaccctgccagaaacaattcagcctcaacaagccgagttgtgcacaagggagactaattttttagacgggaagcaacccgcttccagaagcatccggacgggggtagcgaggctgtgtcgagcgccgtggggatctggccgatgaggtgcccgaaacccgtgtacagctcagcggctgggatcatcgacccccgggatcatcgaccccgtgggccgggcccccggaccctataactaaaagccgacgccagcgcaaaaccacaaacatttactccttaatcctcgctcctccttcatatacaaccacaagtaatcaactcaccaatgtccctggtgaccaagttcctgtctctgccgtcgtttctcttttcggtgttcatcttttactggtccctgcctatcttcgccattccataccgcatccgcttcgctaacgtgggcaagcggaacgacatgctcgactgggcgaaggctctggtcggcttctttcgcgtgacggtcctgaagcccggcgcgacctccgtgtaccgcgggggtccgtgcctgtacctctgcaaccatcgctcctgggccgactttttcatcgacgtgtacctgacccagggccagtcggccctcatgtcgcggtggctggtgttctttgtctttcccgtgttctgtacctcctgcctgatcctccgcggcatcgtcctctttaagcggggcacgatcctggacaaggaggcgtttaacgcctggctcgatgctacgcgcgcccgaagcatcgtgccgggcctggtggtctacccggaggggcatcggtccctgaagccgacctcgctgccgctgaagcgcgggatgatccactacgccttctctcggcgcctgccagtccaactgatcgtgacctcgggcaaggagcatgtgctgagcgagaagacgatgtcggtcaagtttggcaagaccctcgtgacctcgtactcgaagccgctgaagcctagcgattacgcgacgtttgatgccttcatggccgacatccagtcgaactgggaccagtgctggaaggaggcgtacaccgctcccgctcgcggcatgccagaggtctccatggcgggcgcgcactcttacgcctacccacgcctgacctggtgcctccagctcgccatcaccgtcgtgtccatcgtgatcctgggaggggtgctggcgggctcgtggcggggcgccgcggccgtcctcgctgccgcgggccccgtcggccaacaggtgctcgtctccctgctcgccctgtgggcgggggtctccctcctgcgatcgttcctctgagtaccacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccccggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 71 - DNA, 6259 bp, synthetic construct
  source 1..6259: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggactttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcattcgccgctcagcctacacgtcttctccgatacctttccctcattgcattttatgccagactgggtcccagcctgggtgggtgctcccgctcgattgctcgtgtcggaggcggggcacccccgctctctctatttatcactgcctctccccgaccaaccctgacgactgtaaccctgccagaaacaattcagcctcaacaagccgagttgtgcacaagggagactaattttttagacgggaagcaacccgcttccagaagcatccggacgggggtagcgaggctgtgtcgagcgccgtggggatctggccgatgaggtgcccgaaacccgtgtacagctcagcggctgggatcatcgacccccgggatcatcgaccccgtgggccgggcccccggaccctataactaaaagccgacgccagcgcaaaaccacaaacatttactccttaatcctcgctcctccttcatatacaaccacaagtaatcaactcaccaatgagcattacctcgaaggctctgagcctgccttccttcctgttcaccgtgttcgtcttctactggtccctgcctctcttcgccattatctaccgcatccgctttgccaacgtgggcaagcggaacgatatgctggattgggctaaggcgctcgtcgcctttttccgcgtcaccgtcctgaagcagggtgagatgtccctgtaccgcggaggccgctgcctgtacctgtgcaaccaccgctcctgggccgactttttcatcgacacctacctgacggagggacgcgccgcgctcatgtctcggtggatggtgtttttcgcctttcccgtgttttccacgtccgtcctgatcctgcgcggcatcgtcctgtttaagcggggccatatcgccgacaaggaggcctttaactcttggctcgacgccaccctgcggaagtcgacggtgccagccctgctcgtctacccggagggacaccgctctctgaagccgcgctccctgcccctcaagcgcggtatgctgcatttcgcgttctcccgccagctcgcggtgcagatggtcatcacgcgaggcaaggaggaggtgctgagcgagaagacgtgctcggcccgctggggccgcaccctggtcaccacgtactccaaggtgatcaagcccacggactacgactcctttgacgcttttttcaacgagatccaaaccacgtgggacgcctgctggcaactgtcgtaccaccagcccaccgagggcgtgccccaactgtcgctgaagggcgcgcacgagtacgactacccggtgaacatgtggtggctgcaactgggcgtcacgctcctgtccatcacgatcctggtggcggtcctgtacgggagctgggtggcgctgcgcgcgggcgtctcttccctgggcagctccatcacgcaacaggcggtggtcctgggcgtggcgggctggctcggcgtgtccctcctgcggtcgttcctgtgagtaccacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccccggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 72 - DNA, 6256 bp, synthetic construct
  source 1..6256: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggactttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcattcgccgctcagcctacacgtcttctccgatacctttccctcattgcattttatgccagactgggtcccagcctgggtgggtgctcccgctcgattgctcgtgtcggaggcggggcacccccgctctctctatttatcactgcctctccccgaccaaccctgacgactgtaaccctgccagaaacaattcagcctcaacaagccgagttgtgcacaagggagactaattttttagacgggaagcaacccgcttccagaagcatccggacgggggtagcgaggctgtgtcgagcgccgtggggatctggccgatgaggtgcccgaaacccgtgtacagctcagcggctgggatcatcgacccccgggatcatcgaccccgtgggccgggcccccggaccctataactaaaagccgacgccagcgcaaaaccacaaacatttactccttaatcctcgctcctccttcatatacaaccacaagtaatcaactcaccaatgtccctgatctcgaaggctctgtcgctcccgtctttcctcttttcggtcttcgtgttctactggtcgctgccttttttcgccaccacgtaccgcatccgcttcgccaacgtcggtaagcgaaacgacatgctggactgggctaagacgctggtcgcgtttttccgcgtcaccgtcctgaagcagggcaacatgtccctctaccgtgggggccgctgcctgtacctgtgcaaccaccgctcctgggccgactttttcatcgacacctacctgacggagggccgcgcggccctgatgtcccggtggctcgtgtactttgtcttccccgtgttctgcacgagcgtgattatcctccgagggatcgtgctgtttaagcgtggccacatcgcggacaaggaggcttttaacgcgtggctcgacgccacgttctcccgcagcatcgtgccgggcctcctggtgtaccccgaggggcaccgctccctgaagcgcacctctctgcccctgaagcggggtatgatgcactacgcttactcgcgccgaatccccgtgcaactgatcgtgacccgcggcaaggaggaggtcctgtcggagaagaccctctcggcgcggtggggccgaaccctggtcacgacctacagcaaggtcctgaagccgtccgactacgactccttcgacgccttctttaacgacatccaggcgcagtgggacgcctgctggaagacgagctacgacaccccagccgcgggcgtcccgcagctctccatggtcgatgcccacgagtacgactacccccgtcgaatgtggtggctgcaactgggcatgaccctcctgtccatcgtgatcctggccgcggtcctgtggggctcctggctgggcatccgtgcgggcctggccacgctgggctccctccagcaacaggccgtcgtgtggggcgcttccggctggctgggcgcctcgctcctgctctcgttcttctgagtaccacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccccggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 73 - DNA, 6253 bp, synthetic construct
  source 1..6253: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggactttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcattcgccgctcagcctacacgtcttctccgatacctttccctcattgcattttatgccagactgggtcccagcctgggtgggtgctcccgctcgattgctcgtgtcggaggcggggcacccccgctctctctatttatcactgcctctccccgaccaaccctgacgactgtaaccctgccagaaacaattcagcctcaacaagccgagttgtgcacaagggagactaattttttagacgggaagcaacccgcttccagaagcatccggacgggggtagcgaggctgtgtcgagcgccgtggggatctggccgatgaggtgcccgaaacccgtgtacagctcagcggctgggatcatcgacccccgggatcatcgaccccgtgggccgggcccccggaccctataactaaaagccgacgccagcgcaaaaccacaaacatttactccttaatcctcgctcctccttcatatacaaccacaagtaatcaactcaccaatgggtgcggccggaaagctgctctccctcccatcctttctgttcagcgtgtttgtcttttactggagcctgccaatcttcgcgctggtgtaccgtatccgattcgccagctccggccgacgcaacgacatgctcgactgggctaagagcatcgtccggttctttggcgtgaccgtcgtgaaggtgggcgagaacagcctgtaccgcgaggggccatgtctgtacctgtgcaaccaccgctcgtgggccgattttttcatcgacgcttacctcaccgagggacgagcggccctgatgtcgcgctggctggtgtttttcgtgtttcccgtgttctgcacctcctgcctgatcctgcgcggcatcgtcctcttcaagcggggcaccatcgccgacaaggagggcttcaaccgctggctcgactccaagctgggctcctcgcacgtgcctggtctcctggtctaccctgagggccaccgcagcctccgcccctcttcgctgccgctgaagcgcggtatgctccactacgcctacggccggaagctcgccgtgcagattatctgcacggcggggaaggagggggtgatgtcggagaagaccatggccgtgcacttcggccggacgctcgtcacgaccttcagccaggtcctgcgcccatctgagtaccccaccttcgacgcctttttcgcggccgtgcagtccgcgtgggatgcgagctggaaggccgcgaaccagaccccctcggcgggcctgccccgcctgtccatggagggagcgcaggactacgactacccgcccgcgatgcgcgcgcagcaactgggagtgaccctcctgagcttcgtggtcttcggaggcgccctgctcgtcacgtggcgactcctggctgccctcctggcggctgcgggtgcggcccaacaggctgtcgccctgctcctggcgggctggacgggcgtctctatgctccgcagcttcctgtgagtaccacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccccggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 74 - DNA, 6259 bp, synthetic construct
  source 1..6259: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggactttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcattcgccgctcagcctacacgtcttctccgatacctttccctcattgcattttatgccagactgggtcccagcctgggtgggtgctcccgctcgattgctcgtgtcggaggcggggcacccccgctctctctatttatcactgcctctccccgaccaaccctgacgactgtaaccctgccagaaacaattcagcctcaacaagccgagttgtgcacaagggagactaattttttagacgggaagcaacccgcttccagaagcatccggacgggggtagcgaggctgtgtcgagcgccgtggggatctggccgatgaggtgcccgaaacccgtgtacagctcagcggctgggatcatcgacccccgggatcatcgaccccgtgggccgggcccccggaccctataactaaaagccgacgccagcgcaaaaccacaaacatttactccttaatcctcgctcctccttcatatacaaccacaagtaatcaactcaccaatgggtgcggcctcgaagctcctgggcctgccatccttcctgttctccgtcttcgtcttttactggtccctgcccatcttcgccatcgtctaccgcatccgcttcgcttccccagggcggcgcaacgacatgctggactgggccaagtcgattgtgggttttttcggcgtggccgtccacaaggcgggtacgaacagcctgtaccgagacggcccgtgcctgtacctctgtaaccaccgctcttgggcggactttttcatcgacgcctacctcacggagggccgtgcggctctgatgtcgcgctggctggtctttttcgtgttccccgtgttctgcacgagctgcctgatcctccgcggtatcgtcctcttcaagcgcggcaccatcgcggacaaggaggcctttaatagctggctggacgctacgctcggccgctccatcgtgcccggtctcctggtctaccccgagggccaccgttccctgcgccccgcctccctgccgctgaagcgcggcatgctgcactacgcttttggtcgcaagctgcccgtgcaggtggtctgcaccgctggcaaggagggagtcatgtcggagaagaccatggctgtgcgcttcggccgtcgcctcgtcaccaacttctcccaggtgatccgccctgccgacttcccaacgttcgacgccttcagcgcggccgtccagtccgcctgggacggctgctggaaggcggcctacgagacccccgctgcgggcctccctcgcctgtccatggagggctcgcatgagtacgagtacgactaccccgcccccatgcgcgtcctccaagtctgcatcacgctcctgtccatcgtcctcttcctgggcgtcctgctcggctcctggcgcgcgctggccgctgccctggctgccgcgggcgcggctcagcaagtcgtggtcgccctcctggcgacgtggctgggcgcgtccatgctccgctcttgcctctgagtaccacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccccggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 75 - DNA, 6256 bp, synthetic construct
  source 1..6256: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggactttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcattcgccgctcagcctacacgtcttctccgatacctttccctcattgcattttatgccagactgggtcccagcctgggtgggtgctcccgctcgattgctcgtgtcggaggcggggcacccccgctctctctatttatcactgcctctccccgaccaaccctgacgactgtaaccctgccagaaacaattcagcctcaacaagccgagttgtgcacaagggagactaattttttagacgggaagcaacccgcttccagaagcatccggacgggggtagcgaggctgtgtcgagcgccgtggggatctggccgatgaggtgcccgaaacccgtgtacagctcagcggctgggatcatcgacccccgggatcatcgaccccgtgggccgggcccccggaccctataactaaaagccgacgccagcgcaaaaccacaaacatttactccttaatcctcgctcctccttcatatacaaccacaagtaatcaactcaccaatgtccttcatcagcaaggccgtcggtctgcccagcttcctgttctccgtgttcgtgttctactggagcctgcccatcttcgcgctgacgtaccgcatccgcttcgcgtcgaagggcaagcgcaacgacatgctggagtgggctaaggccctggtccgctttttcggcgtgaaggtgctcacgcagggcgagcactcgctgtaccgcgggggcccgtgcctctacctctgcaaccatcggagctgggccgactttttcatcgacgcctacctgaccgagggccgagctgcgctgatgagccgatggctcgtctacttcgtgttccccatcttctgtacctccgtgatggtcctgcgcgggatcgtcctcttcaagcgtgggaccatcgccgataaggaggccttcaacgcctggctcgacgccaagatgaagtcctcgaccattccggggctcctggtctaccccgaggggcaccgcagcctcaagcgcacgtctctgcccctcaagcggggcatgctccattttgcgttctcgcgcggcctgcccgtccaactcgtcatgtgtgctggcaaggaggaggtcctgtctgagaagcgcatggccgtgcgcttcggtcggaccctcgtcgccacgtactccgaggtcctcaagcccggcaactacccgacgttcgacgccttcttttccggcatccagacggcctgggatagctgctggcaggcggcttacaagcagcctcccgagggtgccgcgcccctgcccatggtgaacacgaccgagtacgactatcccccgcgcatgcgccgtctgcaactgatcgtgaccctcctgtcgatcgtcgtgctggccgcggtgctcgcgggcagcgtcctggtcaccgtggtcgccgctgccgcggtgggccccatgccagcgggcgtggtcgtggccgtgctcgctagctggctcgggggcagcatgctgcgttctttcctctgagtaccacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccccggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 76 - DNA, 6256 bp, synthetic construct
  source 1..6256: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggactttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcattcgccgctcagcctacacgtcttctccgatacctttccctcattgcattttatgccagactgggtcccagcctgggtgggtgctcccgctcgattgctcgtgtcggaggcggggcacccccgctctctctatttatcactgcctctccccgaccaaccctgacgactgtaaccctgccagaaacaattcagcctcaacaagccgagttgtgcacaagggagactaattttttagacgggaagcaacccgcttccagaagcatccggacgggggtagcgaggctgtgtcgagcgccgtggggatctggccgatgaggtgcccgaaacccgtgtacagctcagcggctgggatcatcgacccccgggatcatcgaccccgtgggccgggcccccggaccctataactaaaagccgacgccagcgcaaaaccacaaacatttactccttaatcctcgctcctccttcatatacaaccacaagtaatcaactcaccaatgggcgccgcgtcgaaggtgctgggcctgccctcctttctcttctccgtcttcgtcctgtactggtctctgcccatcttcgcggtgctctaccgcatccgattcgccaagatgatcggcaagcgcaacgacgtgctggactgggccaagtctctcgtgcggtttttcggcatcaccgtcctgcgccagggccaagagcaactgtacacgggtggcccctgcctgtacctgtgcaaccaccgctcgtgggccgatttctttctggacgtgtacctgaccgagggccacgcggccgtcatgtcgcgctggctggtctacttcgtcttcccggtcttctgcacgtcctgcctgatcctcaagacgattgtcctcttcaagcgcggcaccatccgcgacaaggagggctttaaccgctggctggacgccgtcctggcccgctcgaacgtgcctggcctcctggtgttcccggagggccaccgcagcctgcgtcccgcctcgctgccactgaagcggggcatgctgcattacgcctacggccggaagctggcgatccagatggtggtcacgcgtggcaaggaggaggtgatgtccgagaagaccatggccgtccgcttcggccgcacgctggtcacgacctactccaaggtcctgcgcccggccgactaccctgactttgaggcgttctttgcggacctccagtccacctgggacggctgctgggaggccgcgtaccggaagcctcgggaggacgccgtgcggcactctctgggcgcggcccccggctttcactacccctttgcgacgcgtgtggcgcaactgggcgccaccctggggtccatcgccctgttcggcctggtcctggtgggctgctggcgtggcctggccgctgccctggcggccacgggcgccgcgcagcaagcggtcgccgcgctcctggccgtctgggtgggtggctccatgctccgttcgttcctgtgagtaccacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccccggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 77 - DNA, 6439 bp, synthetic construct
  source 1..6439: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggactttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcattcgccgctcagcctacacgtcttctccgatacctttccctcattgcattttatgccagactgggtcccagcctgggtgggtgctcccgctcgattgctcgtgtcggaggcggggcacccccgctctctctatttatcactgcctctccccgaccaaccctgacgactgtaaccctgccagaaacaattcagcctcaacaagccgagttgtgcacaagggagactaattttttagacgggaagcaacccgcttccagaagcatccggacgggggtagcgaggctgtgtcgagcgccgtggggatctggccgatgaggtgcccgaaacccgtgtacagctcagcggctgggatcatcgacccccgggatcatcgaccccgtgggccgggcccccggaccctataactaaaagccgacgccagcgcaaaaccacaaacatttactccttaatcctcgctcctccttcatatacaaccacaagtaatcaactcaccaatgcgccagctcaagtccctgatccaactgccctacttcctgttttgtgtcttcgtgttctactggtccttccccatcggctgcctcacccatcacatccacttcctgaagttttccggcaagcgcaacgacatgcacacctggtctgagaccatgtgctctttctttggcatccgcttcgtgaagtacggtgaggagaagctcagcaagggttcccgcaacatcctctacctgtccaaccaccgctcgtgggccgactttttcgtcgatgtctacctgaccgagggcaacgccgcgatgctcagccgcatgctggtcttctttgtctttcccatcttcatgacctcggtcgtggtcctgaagggcattatcctgttcaagcgcggcacgatcgccgacaaggagaagttcaacgcctggctggatgtgatgctggaacgatcgcccatccccggaatgctggtctaccccgagggccatcggtcgacgcggccccacagcctcccgctgaagcgcggcatgctgtactacgcccacagccggggcatgcccgtgcagatcattatctcgaacggcaaggagggcgtcatgtcggagaagcagcgccgagcgggctttggcaagacggtcgtggtcggcttctcttcggtgattgacaccgcggcctacaagggcaacgtcgagggctttatggcgcgagtgcaggaggagtggaacaagctgtggggtcaggtctactccgccgacccggcgggcctgccccctctgcgccaagatcccccgatgctggactacgcggccagcatgcgaacgcagcaactggccatcgccgtcttctccatgttcgccctggctggcgtgctgtgggtgacgtggcgcatgttcgcgtggctgaccgccctggccgggcccctgcaacacgccctggccgtgctcctgctcacctggttcgctgcgtccattatcgcgggcgtgatgggcccgtcgctcccgacccactcttcctcgctccagaatcccccttctctgaaggccgacaagagcgtgtcggccgctgccgctgccgctgccgcggcctccggcgacgggagcctccacgaggcccctagcggcaaggccccggctgccgctgccacccagaaccacaaccaccaacactgagtaccacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccccggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 78 - DNA, 6286 bp, synthetic construct
  source 1..6286: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggactttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcattcgccgctcagcctacacgtcttctccgatacctttccctcattgcattttatgccagactgggtcccagcctgggtgggtgctcccgctcgattgctcgtgtcggaggcggggcacccccgctctctctatttatcactgcctctccccgaccaaccctgacgactgtaaccctgccagaaacaattcagcctcaacaagccgagttgtgcacaagggagactaattttttagacgggaagcaacccgcttccagaagcatccggacgggggtagcgaggctgtgtcgagcgccgtggggatctggccgatgaggtgcccgaaacccgtgtacagctcagcggctgggatcatcgacccccgggatcatcgaccccgtgggccgggcccccggaccctataactaaaagccgacgccagcgcaaaaccacaaacatttactccttaatcctcgctcctccttcatatacaaccacaagtaatcaactcaccaatgggccagtccaccctccagaagctcatttccctgccgacgttctttctctcgatcttcctgatgtactggagcctgcccctctgcaccctgtgtggctacctgcgcctcctggactttaagtccacgcgcaaggacatgtacaactggtgccgcttcatgacgcgtctcttccgtatcaaggtcaagaagtacggctccaagtcgctccgcaaggacgcgggtcggcatatcctgtacctgtgcaaccatcgtagctggacggactttttcctcgacgcttacctgaccgagggcaacgcctccctgatgtcccgttggctcgtgtttgcggtctttccggtcttcatcgtgtcggtgatcgtcctccggggagtcatcctcttcaagcgcggcaccattctggacaaggagaagtttaatcggtggctggactcgaagatcctgtcgaccccgatccccggcgtcctcgtctaccccgagggccaccgctctctggccgacaagtcgctgccactcaagcgcggcatgctctactacgcccactcccgaaagttcccagtgcaaatcattatctcccgcaacaaggaggacgtcctgagcgagaagaccatgaaggcccacttcggcgccacgattatcacgggctgctccgacctgatcgagtctgacggcaaggacatcaaggtctttctgtccgagatccaggccgcgtgggatgacctgtgggagaaggtgtacgcggccgacgcgaccgctctgccacccctggagcagtcgaacatccccgagttcgactacagcatggggatgcgtgtcaagcagctcctggtcgtgtgcagctcggtgctggtgtttctggcggccatggtcgtgtttggtaagctctcttgcctgatctacgccgcgatgggtgacaccgagcgcaaggtcctggtgaccctcatcctgtacgtctggtttttcgtgagcatgctcaacagccgggccccgagcgccacgcagtaggtaccacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccccggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 79 - DNA, 6367 bp, synthetic construct
  source 1..6367: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggactttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcattcgccgctcagcctacacgtcttctccgatacctttccctcattgcattttatgccagactgggtcccagcctgggtgggtgctcccgctcgattgctcgtgtcggaggcggggcacccccgctctctctatttatcactgcctctccccgaccaaccctgacgactgtaaccctgccagaaacaattcagcctcaacaagccgagttgtgcacaagggagactaattttttagacgggaagcaacccgcttccagaagcatccggacgggggtagcgaggctgtgtcgagcgccgtggggatctggccgatgaggtgcccgaaacccgtgtacagctcagcggctgggatcatcgacccccgggatcatcgaccccgtgggccgggcccccggaccctataactaaaagccgacgccagcgcaaaaccacaaacatttactccttaatcctcgctcctccttcatatacaaccacaagtaatcaactcaccaatgtcggcggagaagctgctccacgctccatcctttgtcatcacctgcctggtgctgtactggtccctccctctctccgtgctgttcggctacatccgtgccttccagtggacgggtccccgaaacgacatgtactcttggggccagttcatgtccgacttctttggtgtgaagttcgtgcgcgtcggcaacgagaagctgtacaagccaaagaagggcgcgaacgtcatctacctctgcaaccaccgctcctgggccgactttttctgcgacgtctacctcacccagggcaacgccgctctcctgtcgcgcatgctggtgtttttcatctttccgtttttcatgtccagcgtcgtgatcctgaagggcgtcgtgctgttcaagcggggcaaggtggtcgacaaggacgcgttcaacaagcgtctcgacggcaagctggcggactcgcccatccctggcctcctggtctacccagagggcacgcggagcctgaaggacctgtctctgccgctgaagcgcgggatgctctacttcgcgcatagccgcaacattccggtgcagatcgtggtcagctctggaaaggagtacgtcctcaccgagaagaagatccgcgcccgcatcggccgcaccgtcgtgtccaccttctccgcgaagatcaccccgtccgacttcccatcgtgcgaggccttcctggagcacgtgcaaaaggagtgggaccgcctgtggcccgccgtcttcacggccgacgctgacaaggcccccgtcctggtgaacgaggacatcccgatgcacgactacgcgaacaccatgcgatgggcgcagctcggaatggccgtgctctctaccacggtggcgacgatcatgttcctcctgagctggaaggtcgtggacctgattttcggcatcttcgggcccctgaagcaggtcctgtacatgctcctgttcacctgggtgtcggtgtcgctcgtggcgagcttcctggggccaaagcatctgcccaagggggtcctgccctcccccgtcctgcccctgcccgtggcgcggccaccgcccccgtcccaggagccgtccagctcggggtctaagtgagtaccacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccccggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 80 - DNA, 6304 bp, synthetic construct
  source 1..6304: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggactttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcattcgccgctcagcctacacgtcttctccgatacctttccctcattgcattttatgccagactgggtcccagcctgggtgggtgctcccgctcgattgctcgtgtcggaggcggggcacccccgctctctctatttatcactgcctctccccgaccaaccctgacgactgtaaccctgccagaaacaattcagcctcaacaagccgagttgtgcacaagggagactaattttttagacgggaagcaacccgcttccagaagcatccggacgggggtagcgaggctgtgtcgagcgccgtggggatctggccgatgaggtgcccgaaacccgtgtacagctcagcggctgggatcatcgacccccgggatcatcgaccccgtgggccgggcccccggaccctataactaaaagccgacgccagcgcaaaaccacaaacatttactccttaatcctcgctcctccttcatatacaaccacaagtaatcaactcaccaatggcggccaacatggcgacgttccccttctttctcctgaccttctttttcctgtactggtccatggccatcggcaccctggtcggacgggtcaagttttggcagtacaacggcaccaaccgcaacgagatgttcgtgtgggccaagctgtgctcggactttctgggcgtcaagtacctgaagacgggctcgggctctctctacaagaaggagcgctgcgtgtacctgagcaaccaccgctcctgggcggacttctttgtggacgtgtacctgaccgagggcatcgcgcaggccatggcgcgaaccatggtcggcatcgccttccccatgtttattggcgcggctaccatcctgcgaggcatcctcctgtttaagcggggctccatcgccaacaaggacaagttcaacacctggctcgatgacaagctgaacagctccccgtgctcctctctgctcgtgtttcccgagggccaccgaagcaccaagtccacgagcctgccgctgaagcgcggcatgctgtactacgcgcattcgcgcgggctgcctgtgcaggtgatcatgacgcgcgggaaggagctggtgctgtcggagaagcaccatcacgcgggctacggcacccaactgattacgggcttttccgagctggtgctgagcaaggataaggagttcgagcagttttggaccgaggtccaggcgatgtgggaccgcatgtgggccgaggtgtacagcgtcgacccctccacggcggtgccttacacgcaggacgtgcccggcagcgtgtaccccctctccatgtcggtcctgtacaccgtgtttacggtgtcgaccgtcatctttctgctctcgacgctctggatcatggcgcggatcttcggcttcatgatctccccgctgcccgcgatgctccaaatggtggtctggggcgccgtcccggtcaccctggcggtgtcgttccgggcctctattatcgacaacgccaagacgtcctcgtccgccgtcatcaagaccgcgaagaaggcgcaatgagtaccacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccccggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 81 - DNA, 6289 bp, synthetic construct
  source 1..6289: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggactttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcattcgccgctcagcctacacgtcttctccgatacctttccctcattgcattttatgccagactgggtcccagcctgggtgggtgctcccgctcgattgctcgtgtcggaggcggggcacccccgctctctctatttatcactgcctctccccgaccaaccctgacgactgtaaccctgccagaaacaattcagcctcaacaagccgagttgtgcacaagggagactaattttttagacgggaagcaacccgcttccagaagcatccggacgggggtagcgaggctgtgtcgagcgccgtggggatctggccgatgaggtgcccgaaacccgtgtacagctcagcggctgggatcatcgacccccgggatcatcgaccccgtgggccgggcccccggaccctataactaaaagccgacgccagcgcaaaaccacaaacatttactccttaatcctcgctcctccttcatatacaaccacaagtaatcaactcaccaatggccagcttcgtcgatctcctgctccagttcccctactttggcctgacgatcttctttttctactggtccccacccctggcgtccatcgtctaccgaatccgcttcgcgtcggcgggaccccgcaaggacatctacgagtggacgaccgccatggtccagttcctcggcgtgcgcatccgtcaccaaggcaagcagaccctgtacaagaaggagcgctgcgtctacctgtgcaaccaccgctcctgggcggactttttcgtggacctgcacctgacccaaggcctggcggcccccatgagccgcaccatggtgtttttcgccttccctgtgttcatgagcatttgcatcgtgctcaagggcgtcctcctcttcaagcgcggcacgatcgccgacaagacgaagttcaacaagtggctggatgacagcctgggcaacagcccggtgtcctgcctgctcgtgtaccccgagggccaccgctctacgaaggcgaagtcgctgccgctgaagcgcggtatgctgtactacgcgcacagccgcggcctgcccgtgcagattatccataccgcgggcaaggaggccgtcctgtccgagaagcgcatgcgcgcgagctacggccagaccctcgtcacgggcttctcgcagcccatccagtccaaggacttcaaggacttcgaggccttctctgcggaggtccagcggagctgggacgcgctctggaccgaggtgtactccgcgaacccctccgcgctgcccccgctggtgcaggaggacgtccccaccgcggactacccgccttccatgcgcacgggcatgctcctggtcagcctggcggccatcgccctgttcctggcctttgtcgcgctcctggcctacatcacgggcccggtgatgagcctgttccccgctcccctccgcgtcccactcattctctttaagctgtcctggcacgcggccagcttcctgtactgccgggccaaggacacccctggcaaggtgaaggcggcgtgagtaccacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccccggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 82 - DNA, 6358 bp, synthetic construct
  source 1..6358: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggactttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcattcgccgctcagcctacacgtcttctccgatacctttccctcattgcattttatgccagactgggtcccagcctgggtgggtgctcccgctcgattgctcgtgtcggaggcggggcacccccgctctctctatttatcactgcctctccccgaccaaccctgacgactgtaaccctgccagaaacaattcagcctcaacaagccgagttgtgcacaagggagactaattttttagacgggaagcaacccgcttccagaagcatccggacgggggtagcgaggctgtgtcgagcgccgtggggatctggccgatgaggtgcccgaaacccgtgtacagctcagcggctgggatcatcgacccccgggatcatcgaccccgtgggccgggcccccggaccctataactaaaagccgacgccagcgcaaaaccacaaacatttactccttaatcctcgctcctccttcatatacaaccacaagtaatcaactcaccaatggccctggcgcgctccagctcctcgtccgcgctcctgctctggctgctccgcctgccgtactttctgctcaccatcacctttttctactggtccgccacgatcggcgccctgatctaccgcgtccgcttcctgtctaccgggccgcgcaacgacatgtacgactggaccctgctcatgacctcgttcctgggtctccacttccaccaactgggtgcccaacgcctgcactgcgcgccccgagtcgtgtttctggccaaccaccgaagctgggccgactttttcgtcgacatccacctgacccaggggaccgccgctcccatgtcccgcgcgctggtggcttttgcgtttcctgtgttcatgtgcaccgcctggctcctgcgcggactgattatcttcaagcgcggcgcgatcgaggacaaaaagaagttcaactcgtggctggacgagcagctcgacgcgtccgcgggctcgaacctcctggtgtaccccgagggacaccgcagcacccgacccacctccctcccactgaagcgcggcatgctgtactacgcccactcccgcggcctgcccgtgcaaattatcatgaccgctggcaaggagtctgtcctgtcggagaagcaccagtgcgcgtcctttggccaacacctcctgaccagcttcagccccgtgatctcgtctaaggacaaggacttcgaggtgttcgctacggaggtccagcaacgctgggattcgctgtggcgcgaggtgtttagcacgtcgacccaaggcgtccccctcctgcaccaggccagcgtcccactcttcgactaccctcccgctgtccgcacgcacatggctctcgcttccagcgccttcatcctgatgctgctcctggccgcgggcgcggccgtcctggccgtccgcgctaccctggccctgatgccgcccgtcctccaggtcgtggtcctcgctgccagctccctcctgatgctcgcgtcctttctgacgtcggctgccatcacctcgtccaccacgaccacgaccacgaccacgcgacagccccaggctgtggccgacaagggctcgaaggagtcctaggtaccacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccccggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 83 - protein, 326 aa, Volvulina compacta
MSLISKALSLPSFLLSVFVFYWSLPIFAIPYRIRFANVGKRNDMLDWAKALFAFFRVTVLKQGEQGLYRGGPCLYLCNHRSWADFFVDVYLTEGRAALMSRWLVFFVFPVFCTSCLILRGIVLFKRGTILDKEAFNAWLDATLRRSTVPGLLIYPEGHRSLKPTSLPLKRGMLHFAYSRRLPVQLIVTRGKEDILSEKTMAMKYGRTLVTTYSKVLKPTDYDSFDAFMGDVQTSWDAGWKASYEAPTKGVPQLSMAGAHEYDYPATMRWQQLAITLFSIVVFAAVLYGTWWGLGAVLAAAGPLGQQVVVVLAATSWLGASLIRSCR

SEQ ID NO: 84 - protein, 328 aa, Vitreochlamys sp.
MSLISKASSLPSFLFSVFVFYWSLPIFAVPYRIRFANMGKRNDMLDWAKALVAFFRVTVLKQGEQGLYRGGTCLYLCNHRSWADFFIDVYLTEGRAALMSRWLVFFVFPVFCTSCLILRGIVLFKRGTILDKEAFNAWLDATLRRSIVPGLVVYPEGHRSLKPTSLPLKRGMLHFAFGRQLPVQLIVTRGKEDVLSEKTMAMKYGRTLVTSYSKVLKPSDYASFDAFMTDIQTCWDSCWRAAYEAPTKGVPQLSMVGAHEYDYPRTIWWQQLWITLASILIFAGVLYGTWRGLASLLAAATTSGALGPQVLVTVAAAWAGSSLLRSLL

SEQ ID NO: 85 - protein, 324 aa, Colemanosphaera charkowiensis
MSIVTKVLNLPSFLFSVFVFYWSLPLFAILYRIRFANVGKRNDMLDWAKTLVGFFHVTILKQGELSLYRDGPCLYLCNHRSWADFFIDTYLTEGRAALMSRWLVYFVFPVFCTSVIILRGIVLFKRGSIADKEAFNAWLDATLRKSIVPALLVYPEGHRSLRPTSLPLKRGMLHYAYSRRLPVQLIVTSGKEAVMSEKTMSVRFGRTLVTTYSKVLRPADYPTFDAFFSDIQATWDGCWDAAYKQPSAGAPQLRLVDPHGYNYPARMWWLQLGVTLLSIGVMAAVIAASWWGLGALLAATGPAKQVVLGVVVSLLGASLLRSCL

SEQ ID NO: 86 - protein, 324 aa, Pleodorina japonica
MSVIYKALSLPSFLFSVFVFYWSLPLFASVYRIRFANFGKRNDMLDWAKALVAFFRVTVLKQGEMSLYRGGPCLYLCNHRSWADFFIDTYLTEGRAALMSRWLVYFVFPVFCTSVLILRGIVLFKRGHIVDKEAFNAWLDATLFKSTVPGLLVYPEGHRSLKPASLPLKRGMLHFAFSRKLPVQVICTRGKEEVLCEKTLSARWGRTLVVTFSKVIKPSEYDSFDAFFNDVQSTWDACWQLSYHQPTAGVPELSMKDAHEYDYPRSMWWLQLGITLLSFIILAAVLWGSWVAVRAGFATLGSVQQAVVLGTAGWIGASMVCGFL

SEQ ID NO: 87 - protein, 325 aa, Volvulina boldii
MSALKKALSLPSFLFSVFVFYWSLPIFASVYRIRFARAGKRNDMLDWATSLVAFFRVTVLKQGQFSIYRGGPCIYLCNHRSWADFFIDMYLTEGRAAFMSRWMVFFVFPVFCSAVLILKGIILFKRGRILDKEAFNAWLDATLAKSTIPGLVVYPEGHRSLKPTSLPLKRGMLHYAFSRKLPVQLIVTRGKEEVLSEKTLSMRFGRTLVTSYSKVMKPSDYGSFDAFMTDVQSGWDQCWKEAYSAPTHGLPQLSMAGATEYDYPASMRWQHLAMTLFSIVVFAGVLAASWWGVGALLSAAGPTGKQVVVLLASCWFGASLLRSFL

SEQ ID NO: 88 - protein, 325 aa, Pandorina morum
MSLVTKFLSLPSFLFSVFIFYWSLPIFAIPYRIRFANVGKRNDMLDWAKALVGFFRVTVLKPGATSVYRGGPCLYLCNHRSWADFFIDVYLTQGQSALMSRWLVFFVFPVFCTSCLILRGIVLFKRGTILDKEAFNAWLDATRARSIVPGLVVYPEGHRSLKPTSLPLKRGMIHYAFSRRLPVQLIVTSGKEHVLSEKTMSVKFGKTLVTSYSKPLKPSDYATFDAFMADIQSNWDQCWKEAYTAPARGMPEVSMAGAHSYAYPRLTWCLQLAITVVSIVILGGVLAGSWRGAAAVLAAAGPVGQQVLVSLLALWAGVSLLRSFL

SEQ ID NO: 89 - protein, 326 aa, Volvox carteri
MSITSKALSLPSFLFTVFVFYWSLPLFAIIYRIRFANVGKRNDMLDWAKALVAFFRVTVLKQGEMSLYRGGRCLYLCNHRSWADFFIDTYLTEGRAALMSRWMVFFAFPVFSTSVLILRGIVLFKRGHIADKEAFNSWLDATLRKSTVPALLVYPEGHRSLKPRSLPLKRGMLHFAFSRQLAVQMVITRGKEEVLSEKTCSARWGRTLVTTYSKVIKPTDYDSFDAFFNEIQTTWDACWQLSYHQPTEGVPQLSLKGAHEYDYPVNMWWLQLGVTLLSITILVAVLYGSWVALRAGVSSLGSSITQQAVVLGVAGWLGVSLLRSFL

SEQ ID NO: 90 - protein, 325 aa, Eudorina cylindrica
MSLISKALSLPSFLFSVFVFYWSLPFFATTYRIRFANVGKRNDMLDWAKTLVAFFRVTVLKQGNMSLYRGGRCLYLCNHRSWADFFIDTYLTEGRAALMSRWLVYFVFPVFCTSVIILRGIVLFKRGHIADKEAFNAWLDATFSRSIVPGLLVYPEGHRSLKRTSLPLKRGMMHYAYSRRIPVQLIVTRGKEEVLSEKTLSARWGRTLVTTYSKVLKPSDYDSFDAFFNDIQAQWDACWKTSYDTPAAGVPQLSMVDAHEYDYPRRMWWLQLGMTLLSIVILAAVLWGSWLGIRAGLATLGSLQQQAVVWGASGWLGASLLLSFF

SEQ ID NO: 91 - protein, 324 aa, Gonium multicoccum
MGAAGKLLSLPSFLFSVFVFYWSLPIFALVYRIRFASSGRRNDMLDWAKSIVRFFGVTVVKVGENSLYREGPCLYLCNHRSWADFFIDAYLTEGRAALMSRWLVFFVFPVFCTSCLILRGIVLFKRGTIADKEGFNRWLDSKLGSSHVPGLLVYPEGHRSLRPSSLPLKRGMLHYAYGRKLAVQIICTAGKEGVMSEKTMAVHFGRTLVTTFSQVLRPSEYPTFDAFFAAVQSAWDASWKAANQTPSAGLPRLSMEGAQDYDYPPAMRAQQLGVTLLSFVVFGGALLVTWRLLAALLAAAGAAQQAVALLLAGWTGVSMLRSFL

SEQ ID NO: 92 - protein, 326 aa, Gonium viridistellatum
MGAASKLLGLPSFLFSVFVFYWSLPIFAIVYRIRFASPGRRNDMLDWAKSIVGFFGVAVHKAGTNSLYRDGPCLYLCNHRSWADFFIDAYLTEGRAALMSRWLVFFVFPVFCTSCLILRGIVLFKRGTIADKEAFNSWLDATLGRSIVPGLLVYPEGHRSLRPASLPLKRGMLHYAFGRKLPVQVVCTAGKEGVMSEKTMAVRFGRRLVTNFSQVIRPADFPTFDAFSAAVQSAWDGCWKAAYETPAAGLPRLSMEGSHEYEYDYPAPMRVLQVCITLLSIVLFLGVLLGSWRALAAALAAAGAAQQVVVALLATWLGASMLRSCL

SEQ ID NO: 93 - protein, 325 aa, Volvox ferrisii
MSFISKAVGLPSFLFSVFVFYWSLPIFALTYRIRFASKGKRNDMLEWAKALVRFFGVKVLTQGEHSLYRGGPCLYLCNHRSWADFFIDAYLTEGRAALMSRWLVYFVFPIFCTSVMVLRGIVLFKRGTIADKEAFNAWLDAKMKSSTIPGLLVYPEGHRSLKRTSLPLKRGMLHFAFSRGLPVQLVMCAGKEEVLSEKRMAVRFGRTLVATYSEVLKPGNYPTFDAFFSGIQTAWDSCWQAAYKQPPEGAAPLPMVNTTEYDYPPRMRRLQLIVTLLSIVVLAAVLAGSVLVTVVAAAAVGPMPAGVVVAVLASWLGGSMLRSFL

SEQ ID NO: 94 - protein, 325 aa, Vitreochlamys aulata
MGAASKVLGLPSFLFSVFVLYWSLPIFAVLYRIRFAKMIGKRNDVLDWAKSLVRFFGITVLRQGQEQLYTGGPCLYLCNHRSWADFFLDVYLTEGHAAVMSRWLVYFVFPVFCTSCLILKTIVLFKRGTIRDKEGFNRWLDAVLARSNVPGLLVFPEGHRSLRPASLPLKRGMLHYAYGRKLAIQMVVTRGKEEVMSEKTMAVRFGRTLVTTYSKVLRPADYPDFEAFFADLQSTWDGCWEAAYRKPREDAVRHSLGAAPGFHYPFATRVAQLGATLGSIALFGLVLVGCWRGLAAALAATGAAQQAVAALLAVWVGGSMLRSFL

SEQ ID NO: 95 - protein, 386 aa, Chlamydomonas sp.
MRQLKSLIQLPYFLFCVFVFYWSFPIGCLTHHIHFLKFSGKRNDMHTWSETMCSFFGIRFVKYGEEKLSKGSRNILYLSNHRSWADFFVDVYLTEGNAAMLSRMLVFFVFPIFMTSVVVLKGIILFKRGTIADKEKFNAWLDVMLERSPIPGMLVYPEGHRSTRPHSLPLKRGMLYYAHSRGMPVQIIISNGKEGVMSEKQRRAGFGKTVVVGFSSVIDTAAYKGNVEGFMARVQEEWNKLWGQVYSADPAGLPPLRQDPPMLDYAASMRTQQLAIAVFSMFALAGVLWVTWRMFAWLTALAGPLQHALAVLLLTWFAASIIAGVMGPSLPTHSSSLQNPPSLKADKSVSAAAAAAAAASGDGSLHEAPSGKAPAAAATQNHNHQH

SEQ ID NO: 96 - protein, 335 aa, Dunaliella salina
MGQSTLQKLISLPTFFLSIFLMYWSLPLCTLCGYLRLLDFKSTRKDMYNWCRFMTRLFRIKVKKYGSKSLRKDAGRHILYLCNHRSWTDFFLDAYLTEGNASLMSRWLVFAVFPVFIVSVIVLRGVILFKRGTILDKEKFNRWLDSKILSTPIPGVLVYPEGHRSLADKSLPLKRGMLYYAHSRKFPVQIIISRNKEDVLSEKTMKAHFGATIITGCSDLIESDGKDIKVFLSEIQAAWDDLWEKVYAADATALPPLEQSNIPEFDYSMGMRVKQLLVVCSSVLVFLAAMVVFGKLSCLIYAAMGDTERKVLVTLILYVWFFVSMLNSRAPSATQ

SEQ ID NO: 97 - protein, 362 aa, Microglena sp.
MSAEKLLHAPSFVITCLVLYWSLPLSVLFGYIRAFQWTGPRNDMYSWGQFMSDFFGVKFVRVGNEKLYKPKKGANVIYLCNHRSWADFFCDVYLTQGNAALLSRMLVFFIFPFFMSSVVILKGVVLFKRGKVVDKDAFNKRLDGKLADSPIPGLLVYPEGTRSLKDLSLPLKRGMLYFAHSRNIPVQIVVSSGKEYVLTEKKIRARIGRTVVSTFSAKITPSDFPSCEAFLEHVQKEWDRLWPAVFTADADKAPVLVNEDIPMHDYANTMRWAQLGMAVLSTTVATIMFLLSWKVVDLIFGIFGPLKQVLYMLLFTWVSVSLVASFLGPKHLPKGVLPSPVLPLPVARPPPPSQEPSSSGSK

SEQ ID NO: 98 - protein, 341 aa, Chlamydomonas sp.
MAANMATFPFFLLTFFFLYWSMAIGTLVGRVKFWQYNGTNRNEMFVWAKLCSDFLGVKYLKTGSGSLYKKERCVYLSNHRSWADFFVDVYLTEGIAQAMARTMVGIAFPMFIGAATILRGILLFKRGSIANKDKFNTWLDDKLNSSPCSSLLVFPEGHRSTKSTSLPLKRGMLYYAHSRGLPVQVIMTRGKELVLSEKHHHAGYGTQLITGFSELVLSKDKEFEQFWTEVQAMWDRMWAEVYSVDPSTAVPYTQDVPGSVYPLSMSVLYTVFTVSTVIFLLSTLWIMARIFGFMISPLPAMLQMVVWGAVPVTLAVSFRASIIDNAKTSSSAVIKTAKKAQ

SEQ ID NO: 99 - protein, 336 aa, Chlamydomonas moewusii
MASFVDLLLQFPYFGLTIFFFYWSPPLASIVYRIRFASAGPRKDIYEWTTAMVQFLGVRIRHQGKQTLYKKERCVYLCNHRSWADFFVDLHLTQGLAAPMSRTMVFFAFPVFMSICIVLKGVLLFKRGTIADKTKFNKWLDDSLGNSPVSCLLVYPEGHRSTKAKSLPLKRGMLYYAHSRGLPVQIIHTAGKEAVLSEKRMRASYGQTLVTGFSQPIQSKDFKDFEAFSAEVQRSWDALWTEVYSANPSALPPLVQEDVPTADYPPSMRTGMLLVSLAAIALFLAFVALLAYITGPVMSLFPAPLRVPLILFKLSWHAASFLYCRAKDTPGKVKAA

SEQ ID NO: 100 - protein, 359 aa, Oophila amblystomatis
MALARSSSSSALLLWLLRLPYFLLTITFFYWSATIGALIYRVRFLSTGPRNDMYDWTLLMTSFLGLHFHQLGAQRLHCAPRVVFLANHRSWADFFVDIHLTQGTAAPMSRALVAFAFPVFMCTAWLLRGLIIFKRGAIEDKKKFNSWLDEQLDASAGSNLLVYPEGHRSTRPTSLPLKRGMLYYAHSRGLPVQIIMTAGKESVLSEKHQCASFGQHLLTSFSPVISSKDKDFEVFATEVQQRWDSLWREVFSTSTQGVPLLHQASVPLFDYPPAVRTHMALASSAFILMLLLAAGAAVLAVRATLALMPPVLQVVVLAASSLLMLASFLTSAAITSSTTTTTTTTRQPQAVADKGSKES

SEQ ID NO: 101 - protein, 324 aa, Edaphochlamys debaryana
MSALNKALGLPSFLFSVFVFYWSLPIFATFYRIRFASNGKRNDVLDWAKSLVRFFGVTVLQQGEHSLYRGGACLYLCNHRSWADFFIDAYLTQGLAALMSRWLVYFVFPVFCTSCMILRGVVLFKRGTIADKDAFNKWLDDTLGSSPVPGLLVYPEGHRSIKPTSLPLKRGMLHYAYSRKKPVQIIITRGKDEILSEKTLAVHFGRTVVTTFSKVLRPADYATFEAFFTELQSTWDSCWVAAYTTPIAGIPRLSMADPHAYDYSSTMRAQQLAITLFSILVLVGVLAGSWRVWTAVLAATGTAQTVVVLVLASWVGTSMLRSFL

SEQ ID NO: 102 - protein, 326 aa, Volvulina steinii
MSFLGKALSFPAFLFSVFVFYWSLPIFASVYRIRFVKRGKRNDMLDWAKSLVDFFRVRVLKQGENSLYRGGRCLYLCNHRSWADFFLDMYLTEGRAAFMSRWMVFFVFPVFCSACLVLRGIVFFKRGIIRDKEAFNAWLDATLSRSIVPGLVVYPEGHRSLRPTSLPLKRGMLHFAFDRRLPVQVIVTSGKEGVLSEKTLAMRFGCTLVVSYSKVLKPSEYDTFEAFMSDVQAQWDTCWKAVYGSPTTGAPELSMVGAHQYDYPLSMRVQHVAMTLLSIGIMGAVVIGSWRGWNAVLLALGGPVVQQAVVLLGAAWLGVSLLRSCL

SEQ ID NO: 103 - protein, 351 aa, Gonium octonarium
MTTMREEKWHTQQADVGAKSTASKAERVGATSKLLGFPSFLFSVFVLYWSLPIFAILYQIRFASPGRRNNSLDWAKSLVGFFGVTVVKTGEDSLYRGGPCLYLCNHRSWADFFIDAYLTEGRAALMSRWLVFFVFPVFLTAVRIIRGVVLFKRGAVADKEAFNAWLDATLKRSVVPGLLVYPEGHRSLRPTSLPLKRGMLHFAYGRKLPVQIIVTAGKESVMSEKALAVHFGRALVTKFSRALRPTDYPGFDAFFAAVQSTWDECWKAAYGTPAAGTPRLTIDGAHGYDYPPLMRWQLLVVTLLCILLFGGVLLASWRALAALLAAAGAARRAVVLLLALWVGASASRSFV

SEQ ID NO: 104 - protein, 360 aa, Chlamydomonas nivalis
MARGSGSIGRRPGWLSLLLDLPAFLLAIFILYWSSALGAALGYIRFTKMRGLRDDTFEWSKFMLHAFAVRIVRLGPVLPYRQSPCMYLCNHRSWADFFVDTYCTEGRSALMSRWLVWWVFPVFMSVCRIIGGVVLFKRGAVFDKEKFNAWIDKRLTEVPGHSLLVFPEGHRSISSSSLVLKRGMLHYAHSRKLPVQVVLSAGKEGVLSETRQCVRFGRTVVVTYADVIKSGDFPNFDDFLATVQSTWEEKWRQVHSAPKDGLPALDPVGMSFEYTPTTRWRHVAATLSSYVFMAVVLYASWQAAAYALLAMGPLAPVISTILVVWSAVSLTLAWLPERTPSARHWAAPATNGPVASSPVD

SEQ ID NO: 105 - protein, 326 aa, Pycnococcus provasolii
MSLVTEILLRITFCVDLFLLQIVIPLLALLYSPRDAGTHNMSAWTSKMLGYRIHKAGQNTCYVPEEKSTRVMYLANHRSWGDFIVDRLLTGFRGGSLSRIMVIVGIPMPATLAWLGSGQRGTSALWFFKRAKGATPALWATLDRNFEVAIEKAVVVYPEGHRNRSDKPLPIRTGTLRYAFDRNVHVQVLIAEGKERVINEAQLVTRRSPNVVTVSFSDPIVPKNYSTFEGFHGAVCEAWRTTWLAAYGAAAEAGYCAPPPALGPDGMAESATAADHKRIHRPPAAVPWTPPNPGPPPNQHASLGMHMARLCIVGAISAAAYYTLYE

SEQ ID NO: 106 - protein, 334 aa, Bathycoccus prasinos
MGLSSPSKKLESPSSSRDKYVDTRSLAEKGVHLIALLIAFPILAFSYPLSLLTRGTLHGVWNFSKEIRPPITQIWADFMTSNVMNVRFKIVSQKQLYRDGKCVYLCNHRAWADFFIDMSLCEGRSFTLSRMLVMYVFPVFMVPAVFVGAVFPFRRDKGGGHEELNNSIDKHWSRFEQYFPGFLCYPEGTRNIKPHSLPLKRGMLRYAHSRKLPVQIIICAGKEKVLSQKRFAAERNHVLPVAYSSVIHSDSYPVFEDFAEAIRHSWADEWANAYNHENKMEKLKDFKPSERTIRYERWVDFFTTGTVVSFFVFCALVANVLQIIFPKIIELGHA

SEQ ID NO: 107 - protein, 7 aa, unidentified
NHRSWAD

SEQ ID NO: 108 - protein, 7 aa, synthetic construct
NHRSWAD

SEQ ID NO: 109 - protein, 7 aa, synthetic construct
NHRSWTD

SEQ ID NO: 110 - protein, 8 aa, unidentified
GIVLFKRG

SEQ ID NO: 111 - protein, 8 aa, synthetic construct
GIVLFKRG

SEQ ID NO: 112 - protein, 8 aa, synthetic construct
GVLLFKRG

SEQ ID NO: 113 - protein, 8 aa, synthetic construct
GIILFKRG

SEQ ID NO: 114 - protein, 8 aa, synthetic construct
GVIFIDRK

SEQ ID NO: 115 - protein, 8 aa, synthetic construct
GLIIFKRG

SEQ ID NO: 116 - protein, 7 aa, synthetic construct
GVILFKR

SEQ ID NO: 117 - protein, 8 aa, synthetic construct
GILLFKRG

SEQ ID NO: 118 - protein, 8 aa, synthetic construct
GVVLFKRG

SEQ ID NO: 119 - protein, 7 aa, synthetic construct
GIVLFKR

SEQ ID NO: 120 - protein, 13 aa, unidentified
LLLYPEGTRNTKP

SEQ ID NO: 121 - protein, 13 aa, synthetic construct
LLIYPEGHRSLKP

SEQ ID NO: 122 - protein, 12 aa, synthetic construct
LLVYPEGHRSTK

SEQ ID NO: 123 - protein, 12 aa, synthetic construct
LLVYPEGHRSLR

SEQ ID NO: 124 - protein, 12 aa, synthetic construct
LLVYPEGHRSLK

SEQ ID NO: 125 - protein, 12 aa, synthetic construct
LVVYPEGHRSLK

SEQ ID NO: 126 - protein, 12 aa, synthetic construct
MLVYPEGHRSTR

SEQ ID NO: 127 - protein, 12 aa, synthetic construct
MLVYPEGHRSIK

SEQ ID NO: 128 - protein, 12 aa, synthetic construct
LLVYPEGHRSTR

SEQ ID NO: 129 - protein, 12 aa, synthetic construct
LLVFPEGHRSLR

SEQ ID NO: 130 - protein, 12 aa, synthetic construct
MLVYPEGHRSLA

SEQ ID NO: 131 - protein, 12 aa, synthetic construct
VLVYPEGHRSLA

SEQ ID NO: 132 - protein, 12 aa, synthetic construct
LLVFPEGHRSTK

SEQ ID NO: 133 - protein, 12 aa, synthetic construct
LLVYPEGTRSLK

SEQ ID NO: 134 - protein, 11 aa, synthetic construct
WVFPEGTRNHN

SEQ ID NO: 135 - protein, 11 aa, unidentified
PFQIIVTTNKE

SEQ ID NO: 136 - protein, 11 aa, synthetic construct
PVQLIVTRGKE

SEQ ID NO: 137 - protein, 11 aa, synthetic construct
PVQIIHTAGKE

SEQ ID NO: 138 - protein, 11 aa, synthetic construct
PVQLIVTSGKE

SEQ ID NO: 139 - protein, 11 aa, synthetic construct
PVQLVMCAGKE

SEQ ID NO: 140 - protein, 11 aa, synthetic construct
PVQVVCTAGKE

SEQ ID NO: 141 - protein, 11 aa, synthetic construct
AVQMVITRGKE

SEQ ID NO: 142 - protein, 11 aa, synthetic construct
PVQIVVTRGKD

SEQ ID NO: 143 - protein, 11 aa, synthetic construct
PVQIIITRGKE

SEQ ID NO: 144 - protein, 11 aa, synthetic construct
PVQVIITRGKD

SEQ ID NO: 145 - protein, 11 aa, synthetic construct
PVQIIISNGKE

SEQ ID NO: 146 - protein, 12 aa, synthetic construct
LPVQVICTRGKE

SEQ ID NO: 147 - protein, 11 aa, synthetic construct
AVQIICTAGKE

SEQ ID NO: 148 - protein, 11 aa, synthetic construct
PVQIIMTAGKE

SEQ ID NO: 149 - protein, 12 aa, synthetic construct
LPVQLIVTSGKE

SEQ ID NO: 150 - protein, 11 aa, synthetic construct
AIQMVVTRGKE

SEQ ID NO: 151 - protein, 11 aa, synthetic construct
PVQIIISRNKE

SEQ ID NO: 152 - protein, 11 aa, synthetic construct
PVQVIMTRGKE

SEQ ID NO: 153 - protein, 11 aa, synthetic construct
PVQIVVSSGKE

SEQ ID NO: 154 - protein, 10 aa, synthetic construct
PIVPIVMSSY

SEQ ID NO: 155 - protein, 302 aa, Chlamydomonas reinhardtii
MARKSSLAQAAPVAPSPSAKPELPARSALVCHAAAASVPLPNSDSAPQPNVLLAKIRAIMFFAWSFLLSLPLFVTMMVMAPLVLAFDKYRRLAQHFVNNLWACASTAPFYKVTIIGRENLPPPDKPVVYVANHQSFLDIYSLFHLQRPFKFISKTSNFLIPIIGWSMFLTGHVMINRVDRRSQLKCLQQCRDLLAEGAPVLFFPEGTRSLDCKMAGFKKGAFSVAAKAGVEVVPITLLGTGSLMPSGKESQLRPGQVTIVVHKALPPNKNADQLCDAARQAVASSLPPELVGSATEMAPDEQ

SEQ ID NO: 156 - protein, 258 aa, Brassica napus
MSDLSGAATPESTYPEPEIKLSSRLRGICFCLVAGISAIVLIVLMIIGHPFVLLFDRYRRKFHHFIAKLWASISIYPFYKTDIQGLENLPSSDTPCVYVSNHQSFLDIYTLLSLGQSYKFISKTGIFVIPVIGWAMSMMGVVPLKRMDPRSQVDCLKRCMELVKKGASVFFFPEGTRSKDGRLGPFKKGAFTIAAKTGVPVVPITLMGTGKIMPTGSEGILNHGDVRVIIHKPIYGSKADVLCEEARNKIAESMNLLS

(19) United States
(12) Patent Application Publication
(10) Pub. No.: US 2025/0290105 A1
(43) Pub. Date: Sep. 18, 2025

(54) REGIOSPECIFIC INCORPORATION OF FATTY ACIDS IN TRIGLYCERIDE OIL

(71) Applicant: Checkerspot, Inc., Alameda, CA (US)

(72) Inventors: Xinhua Zhao, Dublin, CA (US); Xiaoying Zhou, Dublin, CA (US); Mona Correa, Hercules, CA (US); Daniel Gates, Winters, CA (US); Leon H. Parker, IV, San Francisco, CA (US); Scott Franklin, Woodside, CA (US)

(73) Assignee: Checkerspot, Inc., Alameda, CA (US)

(21) Appl. No.: 18/861,314

(22) PCT Filed: Apr. 28, 2023
(86) PCT No.: PCT/US2023/066401

§ 371 Date: Oct. 29, 2024

Related U.S. Application Data
(60) Provisional application No. 63/336,767, filed on Apr. 29, 2022.
(60) Provisional application No. 63/420,188, filed on Oct. 28, 2022.

(51) Int. Cl. C12P 7/6463; A23D 9/007; A23D 9/02; C12N 1/12; C12N 9/10; C12R 1/89

(52) U.S. Cl. CPC C12P 7/6463; A23D 9/007; A23D 9/02; C12N 1/125; C12N 9/1025; C12Y 203/01051; C12R 2001/89

(57) ABSTRACT

Provided herein are triglyceride oil compositions enriched in 1,3-dioleoyl-2-palmitoylglycerol (OPO). Further provided herein are methods of producing non-naturally occurring triglyceride oil compositions enriched in OPO from non-naturally occurring microorganisms and applications thereof in a variety of end products, including, for example, polyols and nutritional supplements.

### CROSS REFERENCE

This application claims the benefit of U.S. Provisional Application No. 63/336,767, filed on Apr. 29, 2022, and U.S. Provisional Application No. 63/420,188, filed on Oct. 28, 2022, each of which is incorporated herein by reference in its entirety.

### SEQUENCE LISTING

The instant application contains a Sequence Listing which has been submitted electronically in XML file format and is hereby incorporated by reference in its entirety. Said XML copy, created on Apr. 27, 2023, is named 50727-729_601_SL.xml and is 480,759 bytes in size.

### BACKGROUND

Oleaginous microorganisms have the ability to convert carbon substrates into oils, including triacylglycerides (TAGs) and lipids, and accumulate these oils intracellularly. Some microorganisms can have capacity to accumulate lipids in amounts of up to 80% dry weight. Thus, oleaginous microorganisms, including microalgae, bacteria, fungi, and yeasts, can serve as a productive source for biobased oil production. Genetic and non-genetic modification techniques can allow for the production of non-naturally occurring oils having particular fatty acid profiles.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Various features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates two TAG species of the same molecular weight showing distinctive daughter ion ratios. Panel A shows OOP and Panel B shows OPO.

FIG. 2 illustrates the mass spectra of OOP/OPO triglycerides from oil produced from various strains of microalgae provided herein. Panel A shows CHK22, D552-1; Panel B shows CHK22, D552-3; Panel C shows CHK22, D552-4; Panel D shows the non-transgenic strain CHK22.

FIG. 3, Panels A-C illustrate amino acid sequence alignments of LPAATs described herein. EcPlsC (SEQ ID NO: 22), OlLPAAT2 (SEQ ID NO: 63), PmLPAAT2 (SEQ ID NO: 24), CosLPAAT2 (SEQ ID NO: 64), CrLPAAT2 (SEQ ID NO: 13), ChsLPAAT2 (SEQ ID NO: 14), CiLPAAT2 (SEQ ID NO: 15), VaLPAAT2 (SEQ ID NO: 21), VcLPAAT2 (SEQ ID NO: 20), NoLPAT4 (SEQ ID NO: 17), NoLPAT3 (SEQ ID NO: 16), PmLPAAT1 (SEQ ID NO: 23), Sll1848 (SEQ ID NO: 19), BnBAT2 (SEQ ID NO: 18), and CrLPAAT1 (SEQ ID NO: 12).

FIG. 4 illustrates schematic diagrams showing structural domains of LPAATs described herein.

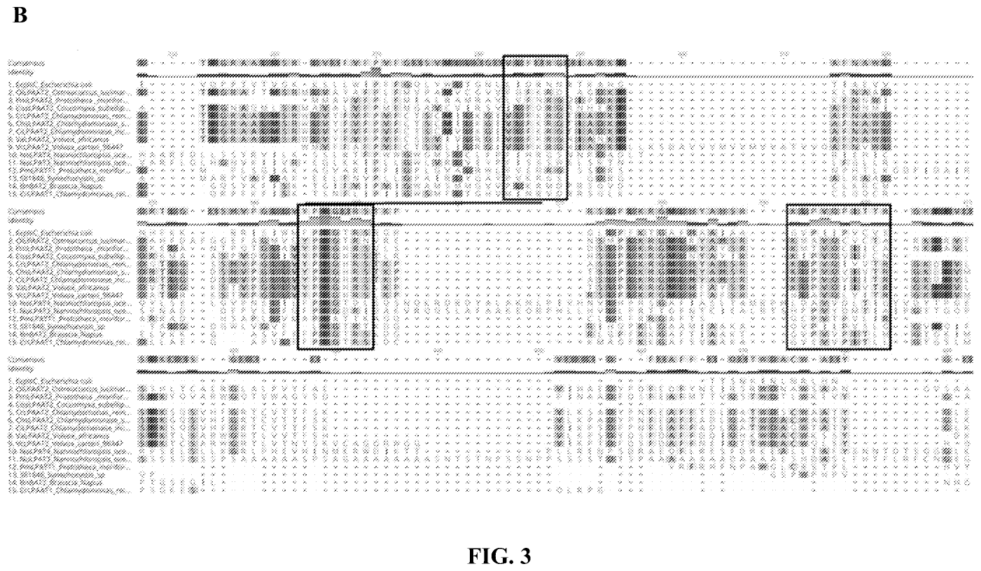

FIG. 5 is a bar graph showing the fatty acid composition at the sn-2 position for oils from various strains, including strains that have been transformed with pCHK385.

FIG. 6 illustrates LC-MS results for oil from CHK22 and strain D552-3.

FIG. 7 illustrates TAG oils enriched in OPO and downstream TAG intermediates generated through chemical conversion processes.

FIG. 8 illustrates an example diol produced from a TAG oil enriched in OPO.

FIG. 9 illustrates an overview of an example classical strain improvement strategy for generation of mutants derived from CHK22 (UTEX 1533).

FIG. 10 illustrates the strain improvement strategy of CHK22 to produce CHK100.

FIG. 11 illustrates transmembrane domains of LPAATs disclosed herein.

### SUMMARY

In some embodiments, provided herein is a non-naturally occurring oil comprising: at least 10 mg of ergosterol per 100 g of the oil; and a triacylglyceride (TAG) component, wherein at least 40% of TAGs in the TAG component are TAG species having a saturated fatty acid at the sn-2 position, wherein at least 50% of the acyl chains in the TAG component are C18:1. The saturated fatty acid can be C16:0. At least 50% of the TAGs in the TAG component can be TAG species having a saturated fatty acid at the sn-2 position. At least 50% of the acyl chains in the TAG component can be C18:1. The TAG species can comprise C18:1 at the sn-1 and sn-3 positions. The TAG species can comprise C16:0 at the sn-2 position. The TAG species can comprise or consists of 1,3-dioleolyl-2-palmitoyl glycerol (OPO). At least 50% of the acyl chains in the TAG component can be C18:1 and at least 20% of the acyl chains in the TAG component can be C16:0. At least 60% of the acyl chains in the TAG component can be C18:1 and at least 30% of the acyl chains in the TAG component can be C16:0. In some embodiments 50-67% or 60-67% of the acyl chains in the TAG component can be C18:1 and 20-33% of the acyl chains in the TAG component can be C16:0. The oil can have an OP:OO m/z ratio of at least 1.6 as determined by abundance of DAG ions resulting from mass spectrometry fragmentation of the TAG component in the oil. The oil can have an OP:OO m/z ratio of at least 2 as determined by abundance of DAG ions resulting from mass spectrometry fragmentation of the TAG component in the oil. The oil can have an OP:OO m/z ratio of at least 3 as determined by abundance of DAG ions resulting from mass spectrometry fragmentation of the TAG component in the oil. The oil can have an OP:OO m/z ratio of at least 4 as determined by abundance of DAG ions resulting from mass spectrometry fragmentation of the TAG component in the oil. The oil can comprise at least 50 mg of ergosterol per 100 g of the oil. The oil can comprise at least 100 mg of ergosterol per 100 g of the oil. The oil can comprise 100-200 mg of ergosterol per 100 g of the oil. The oil can further comprise no more than 5 mg of campesterol, no more than 5 mg of β-sitosterol, or no more than 5 mg of stigmasterol per 100 g of the oil. For example, the oil can comprise no more than 5 mg of campesterol per 100 g of the oil. In some embodiments, the oil does not comprise campesterol. For example, the oil can comprise no more than 5 mg of stigmasterol per 100 g of the oil. In some embodiments, the oil does not comprise stigmasterol. For example, the oil can comprise no more than 5 mg of β-sitosterol per 100 g of the oil. In some embodiments, the oil does not comprise β-sitosterol. The oil can further comprise one or more of ergosta-5,9-dien-3-ol, (3β)-, 5.xi.-ergost-7-en-3β-ol, 9,19-cyclolanostan-3-ol,24-methylene-, (3β)-, and ergosta-7,22-dien-3-ol, (3β). The oil can comprise at least 1 mg of ergosta-5,9-dien-3-ol, (3β)-per 100 g of the oil. The oil can comprise 1-50 mg of ergosta-5,9-dien-3-ol,(3β)-per 100 g of the oil. The oil can comprise at least 15 mg of 5.xi.-ergost-7-en-3β-ol per 100 g of the oil. The oil can comprise 1-50 mg of 5.xi.-ergost-7-en-3β-ol per 100 g of the oil. The oil can comprise at least 20 mg of 5.xi.-ergost-7-en-3β-ol per 100 g of the oil. The oil can comprise at least 5 mg of 9,19-cyclolanostan-3-ol, 24-methylene-,(3β)-per 100 g of the oil. The oil can be an algal oil. The oil can be a genetically modified algal oil. The oil can be produced from an algal cell. The oil can be produced from a genetically modified algal cell. The oil can be produced from a Prototheca cell.

Disclosed herein is a composition comprising the oil and one or more excipients. The composition can be a nutritional supplement. The composition can be an infant formula. The infant formula can comprise one or more of whey, casein, lactose, vitamin D, human milk oligosaccharides (HMOs), vegetable oils, and antibodies. Non-limiting examples of vegetable oils include soy oil, canola oil, sunflower oil, coconut oil, palm oil, and palm kernel oil. The infant formula can comprise soy protein. Disclosed herein is a TAG polyol produced from the oil.

Disclosed herein is a method for producing a TAG polyol, the method comprising: subjecting an oil described herein to epoxidation, thereby generating an epoxidized oil; and ring opening the epoxidized oil in the presence of an alcohol, an acid, or hydrogen and a suitable catalyst, thereby generating the TAG polyol. The epoxidized oil can be ring opened in the presence of an alcohol. The epoxidized oil can be ring opened in the presence of hydrogen and a suitable catalyst. The epoxidized oil can be ring opened in the presence of the acid. Disclosed herein is a TAG polyol produced by a method described herein.

Disclosed herein is a method for producing a TAG polyol, the method comprising: subjecting an oil described herein to hydroformylation, thereby generating a hydroformylated oil; and reducing the hydroformylated oil in the presence of hydrogen and a suitable catalyst, thereby generating the TAG polyol. Disclosed herein is a TAG polyol produced by a method described herein.

Disclosed herein is a microalgal cell comprising an exogenous gene that encodes for an enzyme having lysophosphatidic acid acyltransferase activity, wherein the cell produces an oil comprising a TAG component, wherein at least 40% of TAGs in the TAG component can be TAG species having a saturated fatty acid at the sn-2 position, wherein at least 50% of the acyl chains in the TAG component can be C18:1. The enzyme can be a lysophosphatidic acid acyltransferase (LPAAT). The enzyme can be an algal lysophosphatidic acid acyltransferase (LPAAT).

In some embodiments, an enzyme disclosed herein can comprise a sequence motif selected from any one of (I) NHXXXXD (or NHX₄D); (II) (F/Y)XXR; (III) EGXR; and (IV) Proline, wherein X is any amino acid.

In some embodiments, the enzyme can comprise NHXXXXD (or NHX₄D), wherein X is any amino acid. In some embodiments, the enzyme can comprise a sequence with at least 70%, at least 85%, or 100% sequence identity to any one of SEQ ID NO: 107-109.

In some embodiments, the enzyme can comprise (F/Y)XXR, wherein X is any amino acid. In some embodiments, the enzyme can comprise a sequence with at least 60%, at least 75%, at least 85%, or 100% sequence identity to any one of SEQ ID NO: 110-119.

In some embodiments, the enzyme can comprise EGXR, wherein X is any amino acid. In some embodiments, the enzyme can comprise a sequence with at least 50%, at least 55%, at least 60%, at least 65%, at least 70%, at least 75%, at least 80%, at least 90%, or 100% sequence identity to any one of SEQ ID NO: 120-134.

In some embodiments, the enzyme can comprise a sequence a sequence with a conserved proline. In some embodiments, the enzyme can comprise a sequence with at least 55%, at least 60%, at least 65%, at least 70%, at least 75%, at least 80%, at least 90%, or 100% sequence identity to any one of SEQ ID NO: 135-154.

The enzyme can be Chlamydomonas reinharith lysophosphatidic acid acyltransferase (CrLPAAT1). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 12. The cell can comprise a sequence with at least 80%, at least 90%, at least 95%, at least 96%, at least 97%, at least 98%, at least 99%, or 100% sequence identity to SEQ ID NO: 2.

The enzyme can be Chlamydomonas incerta lysophosphatidic acid acyltransferase (CiLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 15. The cell can comprise a sequence with at least 80%, at least 90%, at least 95%, at least 96%, at least 97%, at least 98%, at least 99%, or 100% sequence identity to SEQ ID NO: 10.

The enzyme can be Chlamydomonas schloesseri lysophosphatidic acid acyltransferase (ChsLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 14. The cell can comprise a sequence with at least 80%, at least 90%, at least 95%, at least 96%, at least 97%, at least 98%, at least 99%, or 100% sequence identity to SEQ ID NO: 9.

The enzyme can be Volvox africanus lysophosphatidic acid acyltransferase (VaLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 21. The cell can comprise a sequence with at least 80%, at least 90%, at least 95%, at least 96%, at least 97%, at least 98%, at least 99%, or 100% sequence identity to SEQ ID NO: 11.

The enzyme can be Nannochloropsis oceanica lysophosphatidic acid acyltransferase (NoLPAT3). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 16. The cell can comprise a sequence with at least 80%, at least 90%, at least 95%, at least 96%, at least 97%, at least 98%, at least 99%, or 100% sequence identity to SEQ ID NO: 7.

The enzyme can be Synechocystis sp. 1-acyl-sn-glycerol-3-phosphate acyltransferase (Sll1848). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 19. The cell can comprise a sequence with at least 80%, at least 90%, at least 95%, at least 96%, at least 97%, at least 98%, at least 99%, or 100% sequence identity to SEQ ID NO: 5.

The enzyme can be Chlamydomonas reinharditi lysophosphatidic acid acyltransferase (CrLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 13. The cell can comprise a sequence with at least 80%, at least 90%, at least 95%, at least 96%, at least 97%, at least 98%, at least 99%, or 100% sequence identity to SEQ ID NO: 1.

The enzyme can be Escherichia coli 1-acyl-sn-glycerol-3-phosphate acyltransferase (EcPlsC). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 22. The cell can comprise a sequence with at least 80%, at least 90%, at least 95%, at least 96%, at least 97%, at least 98%, at least 99%, or 100% sequence identity to SEQ ID NO: 6.

The enzyme can be Volvox carteri lysophosphatidic acid acyltransferase (VcLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 20. The cell can comprise a sequence with at least 80%, at least 90%, at least 95%, at least 96%, at least 97%, at least 98%, at least 99%, or 100% sequence identity to SEQ ID NO: 8. The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 40. The cell can comprise a sequence with at least 80%, at least 90%, at least 95%, at least 96%, at least 97%, at least 98%, at least 99%, or 100% sequence identity to SEQ ID NO: 25.

The enzyme can be Brassica napus acyltransferase (BnBAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 18. The cell can comprise a sequence with at least 80%, at least 90%, at least 95%, at least 96%, at least 97%, at least 98%, at least 99%, or 100% sequence identity to SEQ ID NO: 3. The enzyme can be Nannochloropsis oceanica lysophosphatidic acid acyltransferase (NoLPAT4). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 17. The cell can comprise a sequence with at least 80%, at least 90%, at least 95%, at least 96%, at least 97%, at least 98%, at least 99%, or 100% sequence identity to SEQ ID NO: 4.

The enzyme can be Astrephomene gubernaculifera lysophosphatidic acid acyltransferase (AgLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 41. The cell can comprise a sequence with at least 80%, at least 90%, at least 95%, at least 96%, at least 97%, at least 98%, at least 99%, or 100% sequence identity to SEQ ID NO: 26.

The enzyme can be Edaphochlamys debaryana lysophosphatidic acid acyltransferase (EdLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 42. The cell can comprise a sequence with at least 80%, at least 90%, at least 95%, at least 96%, at least 97%, at least 98%, at least 99%, or 100% sequence identity to SEQ ID NO: 27.

The enzyme can be Dunaliella salina lysophosphatidic acid acyltransferase (DsLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 43. The cell can comprise a sequence with at least 80%, at least 90%, at least 95%, at least 96%, at least 97%, at least 98%, at least 99%, or 100% sequence identity to SEQ ID NO: 28.

The enzyme can be Scenedesmus sp. lysophosphatidic acid acyltransferase (SceLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 44. The cell can comprise a sequence with at least 80%, at least 90%, at least 95%, at least 96%, at least 97%, at least 98%, at least 99%, or 100% sequence identity to SEQ ID NO: 29.

The enzyme can be Micractinium conductrix lysophosphatidic acid acyltransferase (McLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 45. The cell can comprise a sequence with at least 80%, at least 90%, at least 95%, at least 96%, at least 97%, at least 98%, at least 99%, or 100% sequence identity to SEQ ID NO: 30.

The enzyme can be Chlorella sorokiniana lysophosphatidic acid acyltransferase (ChsoLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 46. The cell can comprise a sequence with at least 80%, at least 90%, at least 95%, at least 96%, at least 97%, at least 98%, at least 99%, or 100% sequence identity to SEQ ID NO: 31.

The enzyme can be Chlorella variabilis lysophosphatidic acid acyltransferase (ChvaLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 47. The cell can comprise a sequence with at least 80%, at least 90%, at least 95%, at least 96%, at least 97%, at least 98%, at least 99%, or 100% sequence identity to SEQ ID NO: 32.

The enzyme can be Raphidocelis subcapitata lysophosphatidic acid acyltransferase (RsLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 48. The cell can comprise a sequence with at least 80%, at least 90%, at least 95%, at least 96%, at least 97%, at least 98%, at least 99%, or 100% sequence identity to SEQ ID NO: 33.

The enzyme can be Chlorella desiccata lysophosphatidic acid acyltransferase (ChdeLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 49. The cell can comprise a sequence with at least 80%, at least 90%, at least 95%, at least 96%, at least 97%, at least 98%, at least 99%, or 100% sequence identity to SEQ ID NO: 34.

The enzyme can be Auxenochlorella protothecoides lysophosphatidic acid acyltransferase (ApLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 50. The cell can comprise a sequence with at least 80%, at least 90%, at least 95%, at least 96%, at least 97%, at least 98%, at least 99%, or 100% sequence identity to SEQ ID NO: 35.

The enzyme can be Chloropicon primus lysophosphatidic acid acyltransferase (ChprPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 51. The cell can comprise a sequence with at least 80%, at least 90%, at least 95%, at least 96%, at least 97%, at least 98%, at least 99%, or 100% sequence identity to SEQ ID NO: 36.

The enzyme can be Homo sapiens lysophosphatidic acid acyltransferase (AGPAT1). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 52. The cell can comprise a sequence with at least 80%, at least 90%, at least 95%, at least 96%, at least 97%, at least 98%, at least 99%, or 100% sequence identity to SEQ ID NO: 37.

The enzyme can be Chlamydomonas eustigma lysophosphatidic acid acyltransferase (CeLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 53. The cell can comprise a sequence with at least 80%, at least 90%, at least 95%, at least 96%, at least 97%, at least 98%, at least 99%, or 100% sequence identity to SEQ ID NO: 38.

The enzyme can be a Pedinophyceae lysophosphatidic acid acyltransferase (PedLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 54. The cell can comprise a sequence with at least 80%, at least 90%, at least 95%, at least 96%, at least 97%, at least 98%, at least 99%, or 100% sequence identity to SEQ ID NO: 39. The cell can comprise a sequence with at least 80%, at least 90%, at least 95%, at least 96%, at least 97%, at least 98%, at least 99%, or 100% sequence identity to SEQ ID NO: 59. The cell can comprise a sequence with at least 80%, at least 90%, at least 95%, at least 96%, at least 97%, at least 98%, at least 99%, or 100% sequence identity to SEQ ID NO: 60. The cell can comprise a sequence with at least 80%, at least 90%, at least 95%, at least 96%, at least 97%, at least 98%, at least 99%, or 100% sequence identity to SEQ ID NO: 61. The cell can comprise a sequence with at least 80%, at least 90%, at least 95%, at least 96%, at least 97%, at least 98%, at least 99%, or 100% sequence identity to SEQ ID NO: 62.

The enzyme can be Volvox reticuliferus lysophosphatidic acid acyltransferase (VrLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 55. The cell can comprise a sequence with at least 80%, at least 90%, at least 95%, at least 96%, at least 97%, at least 98%, at least 99%, or 100% sequence identity to SEQ ID NO: 57.

The enzyme can be Chlorella vulgaris lysophosphatidic acid acyltransferase (ChvuLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 56. The cell can comprise a sequence with at least 80%, at least 90%, at least 95%, at least 96%, at least 97%, at least 98%, at least 99%, or 100% sequence identity to SEQ ID NO: 58.

The enzyme can be Volvulina compacta lysophosphatidic acid acyltransferase (VcomLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 83. The cell can comprise a sequence with at least 80%, at least 90%, at least 95%, at least 96%, at least 97%, at least 98%, at least 99%, or 100% sequence identity to SEQ ID NO: 65.

The enzyme can be Vitreochlamys sp. CL-2021 lysophosphatidic acid acyltransferase (VitrLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 84. The cell can comprise a sequence with at least 80%, at least 90%, at least 95%, at least 96%, at least 97%, at least 98%, at least 99%, or 100% sequence identity to SEQ ID NO: 66.

The enzyme can be Colemanosphaera charkowiensis lysophosphatidic acid acyltransferase (CchaLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 85. The cell can comprise a sequence with at least 80%, at least 90%, at least 95%, at least 96%, at least 97%, at least 98%, at least 99%, or 100% sequence identity to SEQ ID NO: 67.

The enzyme can be Pleodorina japonica lysophosphatidic acid acyltransferase (PjapLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 86. The cell can comprise a sequence with at least 80%, at least 90%, at least 95%, at least 96%, at least 97%, at least 98%, at least 99%, or 100% sequence identity to SEQ ID NO: 68.

The enzyme can be Volvulina boldii lysophosphatidic acid acyltransferase (VbolLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 87. The cell can comprise a sequence with at least 80%, at least 90%, at least 95%, at least 96%, at least 97%, at least 98%, at least 99%, or 100% sequence identity to SEQ ID NO: 69.

The enzyme can be Pandorina morum lysophosphatidic acid acyltransferase (PmorLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 88. The cell can comprise a sequence with at least 80%, at least 90%, at least 95%, at least 96%, at least 97%, at least 98%, at least 99%, or 100% sequence identity to SEQ ID NO: 70.

The enzyme can be Volvox carteri f. weismannia lysophosphatidic acid acyltransferase (VcarfLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 89. The cell can comprise a sequence with at least 80%, at least 90%, at least 95%, at least 96%, at least 97%, at least 98%, at least 99%, or 100% sequence identity to SEQ ID NO: 71.

The enzyme can be Eudorina cylindrica lysophosphatidic acid acyltransferase (EcylLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 90. The cell can comprise a sequence with at least 80%, at least 90%, at least 95%, at least 96%, at least 97%, at least 98%, at least 99%, or 100% sequence identity to SEQ ID NO: 72.

The enzyme can be Gonium multicoccum lysophosphatidic acid acyltransferase (GmulLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 91. The cell can comprise a sequence with at least 80%, at least 90%, at least 95%, at least 96%, at least 97%, at least 98%, at least 99%, or 100% sequence identity to SEQ ID NO: 73.

The enzyme can be Gonium viridistellatum lysophosphatidic acid acyltransferase (GvirLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 92. The cell can comprise a sequence with at least 80%, at least 90%, at least 95%, at least 96%, at least 97%, at least 98%, at least 99%, or 100% sequence identity to SEQ ID NO: 74.

The enzyme can be Volvox ferrisii lysophosphatidic acid acyltransferase (VferLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 93. The cell can comprise a sequence with at least 80%, at least 90%, at least 95%, at least 96%, at least 97%, at least 98%, at least 99%, or 100% sequence identity to SEQ ID NO: 75.

The enzyme can be Vitreochlamys aulata lysophosphatidic acid acyltransferase (VaulLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 94. The cell can comprise a sequence with at least 80%, at least 90%, at least 95%, at least 96%, at least 97%, at least 98%, at least 99%, or 100% sequence identity to SEQ ID NO: 76.

The enzyme can be Chlamydomonas sp. CCAC2762_B lysophosphatidic acid acyltransferase (Ch_CCAC2762_LPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 95. The cell can comprise a sequence with at least 80%, at least 90%, at least 95%, at least 96%, at least 97%, at least 98%, at least 99%, or 100% sequence identity to SEQ ID NO: 77.

The enzyme can be Dunaliella salina lysophosphatidic acid acyltransferase (DsalLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 96. The cell can comprise a sequence with at least 80%, at least 90%, at least 95%, at least 96%, at least 97%, at least 98%, at least 99%, or 100% sequence identity to SEQ ID NO: 78.

The enzyme can be Microglena sp. YARC lysophosphatidic acid acyltransferase (MyarcLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 97. The cell can comprise a sequence with at least 80%, at least 90%, at least 95%, at least 96%, at least 97%, at least 98%, at least 99%, or 100% sequence identity to SEQ ID NO: 79.

The enzyme can be Chlamydomonas sp. UWO_241 lysophosphatidic acid acyltransferase (CuwoLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 98. The cell can comprise a sequence with at least 80%, at least 90%, at least 95%, at least 96%, at least 97%, at least 98%, at least 99%, or 100% sequence identity to SEQ ID NO: 80.

The enzyme can be Chlamydomonas moewusii lysophosphatidic acid acyltransferase (CmoeLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 99. The cell can comprise a sequence with at least 80%, at least 90%, at least 95%, at least 96%, at least 97%, at least 98%, at least 99%, or 100% sequence identity to SEQ ID NO: 81.

The enzyme can be Oophila amblystomatis lysophosphatidic acid acyltransferase (OambLPAAT2). The enzyme can comprise a sequence with at least 50%, at least 60%, at least 70%, at least 80%, at least 90%, at least 95%, at least 96%, at least 97%, at least 98%, at least 99%, or 100% sequence identity to SEQ ID NO: 100. The cell can comprise a sequence with at least 80%, at least 90%, at least 95%, at least 96%, at least 97%, at least 98%, at least 99%, or 100% sequence identity to SEQ ID NO: 82.

The exogenous gene can be codon-optimized for expression in a Prototheca strain. In some embodiments, the cell does not comprise an exogenous glycerol-3-phosphate acyltransferase (GPAT1). The cell can be from a Prototheca base strain. The cell can be from the Prototheca moriformis base strain UTEX 1533. The cell can be derived from a classically improved strain from Prototheca moriformis base strain UTEX 1533. The cell can be from a non-genetically modified Prototheca base strain that produces an oil having a fatty acid profile of at least 50% oleic acid. The cell can be from a non-genetically modified Prototheca base strain that produces an oil having a fatty acid profile of at least 30% palmitic acid. The Prototheca base strain can be Prototheca wickerhamii. The Prototheca base strain can be Prototheca moriformis. The microalgal cell can produce at least 60% lipid by dry cell weight. The microalgal cell can produce at least 50% lipid by dry cell weight. The oil can comprise a triacylglyceride (TAG) component, wherein at least 40% of TAGs in the TAG component can be TAG species having a saturated fatty acid at the sn-2 position, wherein at least 50% of the acyl chains in the TAG component can be C18:1. The oil can comprise at least 10 mg of ergosterol per 100 g of the oil. The saturated fatty acid can be C16:0. At least 50% of the TAGs in the TAG component can be TAG species having a saturated fatty acid at the sn-2 position. At least 50% of the acyl chains in the TAG component can be C18:1. The TAG species can comprise C18:1 at the sn-1 and sn-3 positions. The TAG species can comprise C16:0 at the sn-2 position. The TAG species can comprise or consists of 1,3-dioleolyl-2-palmitoyl glycerol (OPO). At least 50% of the acyl chains in the TAG component can be C18:1 and at least 20% of the acyl chains in the TAG component can be C16:0. At least 60% of the acyl chains in the TAG component can be C18:1 and at least 30% of the acyl chains in the TAG component can be C16:0. In some embodiments 50-67% or 60-67% of the acyl chains in the TAG component can be C18:1 and 20-33% of the acyl chains in the TAG component can be C16:0. The oil can have an OP:OO m/z ratio of at least 1.6 as determined by abundance of DAG ions resulting from mass spectrometry fragmentation of the TAG component in the oil. The oil can have an OP:OO m/z ratio of at least 2 as determined by abundance of DAG ions resulting from mass spectrometry fragmentation of the TAG component in the oil. The oil can have an OP:OO m/z ratio of at least 3 as determined by abundance of DAG ions resulting from mass spectrometry fragmentation of the TAG component in the oil. The oil can comprise at least 50 mg of ergosterol per 100 g of the oil. The oil can comprise at least 100 mg of ergosterol per 100 g of the oil. The oil can further comprise one or more of 5.xi.-ergost-7-en-3β-ol, 9,19-cyclolanostan-3-ol,24-methylene-,(3β)-, and ergosta-7,22-dien-3-ol, (3β). The oil can comprise at least 20 mg of 5.xi.-ergost-7-en-3β-ol per 100 g of the oil. The oil can comprise at least 5 mg of 9,19-cyclolanostan-3-ol,24-methylene-,(3β)-per 100 g of the oil. The oil can be an algal oil. The oil can be a genetically modified algal oil. The oil can be produced from an algal cell. The oil can be produced from a genetically modified algal cell. The oil can be produced from a Prototheca cell.

Disclosed herein is a method of producing a non-naturally occurring oil, the method comprising cultivating a microalgal cell in a culture medium, wherein the oil comprises a TAG component, wherein at least 40% of TAG species in the TAG component have a saturated fatty acid at the sn-2 position, wherein at least 50% of the acyl chains in the TAG component can be C18:1. The method can further comprise isolating the oil composition from the culture medium. The method can further comprise expressing in the microalgal cell an exogenous enzyme having lysophosphatidic acid acyltransferase activity. The microalgal cell can be a microalgal cell described herein. The oil can comprise a triacylglyceride (TAG) component, wherein at least 40% of the TAGs in the TAG component can be TAG species having a saturated fatty acid at the sn-2 position, wherein at least 50% of the acyl chains in the TAG component can be C18:1. The oil can comprise at least 10 mg of ergosterol per 100 g of the oil. The saturated fatty acid can be C16:0. At least 50% of the TAGs in the TAG component can be TAG species having a saturated fatty acid at the sn-2 position. At least 50% of the acyl chains in the TAG component can be C18:1. The TAG species can comprise C18:1 at the sn-1 and sn-3 positions. The TAG species can comprise C16:0 at the sn-2 position. The TAG species can comprise or consists of 1,3-dioleolyl-2-palmitoyl glycerol (OPO). At least 50% of the acyl chains in the TAG component can be C18:1 and at least 20% of the acyl chains in the TAG component can be C16:0. At least 60% of the acyl chains in the TAG component can be C18:1 and at least 30% of the acyl chains in the TAG component can be C16:0. 50-67% or 60-67% of the acyl chains in the TAG component can be C18:1 and 20-33% of the acyl chains in the TAG component can be C16:0. The oil can have an OP:OO m/z ratio of at least 1.6 as determined by abundance of DAG ions resulting from mass spectrometry fragmentation of the TAG component in the oil. The oil can have an OP:OO m/z ratio of at least 2 as determined by abundance of DAG ions resulting from mass spectrometry fragmentation of the TAG component in the oil. The oil can have an OP:OO m/z ratio of at least 3 as determined by abundance of DAG ions resulting from mass spectrometry fragmentation of the TAG component in the oil. The oil can comprise at least 50 mg of ergosterol per 100 g of the oil. The oil can comprise at least 100 mg of ergosterol per 100 g of the oil. The oil can further comprise one or more of 5.xi.-ergost-7-en-3β-ol, 9,19-cyclolanostan-3-ol,24-methylene-,(3β)-, and ergosta-7,22-dien-3-ol, (3β). The oil can comprise at least 20 mg of 5.xi.-ergost-7-en-3β-ol per 100 g of the oil. The oil can comprise at least 5 mg of 9,19-cyclolanostan-3-ol,24-methylene-,(3β)-per 100 g of the oil. The oil can be an algal oil. The oil can be a genetically modified algal oil. The oil can be produced from an algal cell. The oil can be produced from a genetically modified algal cell. The oil can be produced from a Prototheca cell.

### DETAILED DESCRIPTION

Provided herein are oil compositions comprising TAGs enriched in 1,3-dioleoyl-2-palmitoylglycerol (OPO), methods of making, formulations, and applications thereof. Oil compositions provided herein can be produced by a genetically modified microorganism provided herein, such as a microalgal cell provided herein. Further provided are methods of producing and cultivating microorganisms that produce the oil compositions provided herein. Further provided herein are bioreactors comprising a non-naturally occurring microorganism provided herein that has been modified to produce the oil compositions provided herein.

### Definitions

As used herein, the term “microbial oil” refers to an oil produced or extracted from a microorganism (microbe), e.g., an oleaginous, single-celled, eukaryotic, or prokaryotic microorganism, including but not limited to, microalgae, yeast, bacteria, and fungi.

As used herein, the term “triacylglycerol”, “triglyceride”, or “TAG” refers to esters between glycerol and three fatty acids.

As used herein, the term “OPO” or “1,3-dioleoyl-2-palmitoylglycerol” refers to a TAG species having a palmitate (C16:0) at the sn-2 position of the TAG and oleate (C18:1) at the sn-1 and sn-3 positions of the TAG.

As used herein, the term “OOP” or “1,2-dioleoyl-3-palmitoylglycerol” refers to a TAG species having a palmitate (C16:0) at the sn-3 position of the TAG and oleate (C18:1) at the sn-1 and sn-2 positions of the TAG.

As used herein, the term “polyol” refers to triglycerols or fatty acid alcohols comprising hydroxyl functional groups. As used herein, the term “polyol derived from a TAG oil” generally refers to a polyol obtained from chemical conversion of a TAG oil, e.g., via epoxidation and ring opening, ozonolysis, and reduction, or hydroformylation and reduction.

As used herein, the term “polyurethane”, “PU”, or “urethane” refers to a class of polymers comprised of carbamate (urethane) linkages formed between a polyol and an isocyanate moiety.

As used herein, the term “high oleic” can refer to greater than 60% oleic acid, greater than 70% oleic acid, greater than 80% oleic acid, or greater than 90% oleic acid.

As used herein, the term “about” refers to ±10% from the value provided.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are described herein.

### Human Breast Milk

Human breast milk is the primary source of nutrition for newborn babies. Breast milk is comprised of fat, protein, carbohydrates, and various vitamins and minerals necessary to support healthy growth and development. Breast milk also contains factors that are important for protecting the infant against infection and inflammation, while also contributing to healthy development of the immune system and gut microbiome.

Breast milk contains about 3-55% fat, and 98% of which is in the form of TAGs containing more than 200 types of different fatty acids. Breast milk contains about 17-25% palmitic acid and 70-75% of which is uniquely distributed in the stereotypically numbered 2 (sn-2) position of TAGs, while unsaturated fatty acids, such as oleic acid and linoleic acid, are mostly distributed in the sn-1 and sn-3 positions. The high content of palmitic acid in the sn-2 position may contribute to enhanced absorption of calcium and essential fats in the infant gut, and overall digestive health. A key TAG in human breast milk is 1,3-dioleoyl-2-palmitoylglycerol, also referred to as sn-2 palmitate, oleic-palmitic-oleic, or OPO. OPO is found in human breast milk, but is generally absent in bovine milk.

Given the complex nature of human breast milk, infant formula manufacturers have struggled to recapitulate even a fraction of the features present in this remarkable, natural food. Some attention has been paid to achieving the relative proportions of desired fatty acids in infant formulas to recapitulate the components of breast milk. Products have also been created that recapitulate TAGs enriched in OPO that can be combined with other fat fractions to mimic human breast milk. Human milk fat substitute (HMFS) is a class of structured lipid that is widely used as an ingredient in infant formulas. Like human milk fat, HMFS is characterized by enrichment of palmitoyl (C16:0) groups specifically at the middle (sn-2 or β) position on the glycerol backbone. However, no single-source fat substitute delivers all the demonstrated benefits of the natural product.

Although the fat used in most infant formulas is derived from plants, many plants esterify saturated fatty acids to the sn-1 and sn-3 positions, and exclude palmitic acid in the sn-2 position. In contrast, engineered oilseed crops and/or oleaginous microorganisms can be used accumulate high levels of TAGs enriched in saturated fatty acid in the sn-2 position, such as OPO. These alternative sources of TAGs may thereby better mimic the stereoisomeric structure of human milk fat and provide improved sources of HMFS for infant nutrition.

### TAG Biosynthesis

Lysophosphatidic acid acyltransferase (LPAAT), a type of membrane-bound o-acyltransferase (MBOAT) encoded by a multigene family, is a rate-limiting enzyme in the Kennedy pathway in higher plants. The Kennedy pathway turns glycerol-3-phosphate into TAG using glycerol-3-phosphate acyltransferase (GPAT), LPAAT, phosphatidic acid phosphatase (PAP), and diacylglycerol acyltransferase (DGAT). In this pathway, LPAAT controls the production of phosphatidic acid (PA), a key intermediate.

LPAAT genes are present in both prokaryotes and eukaryotes. In mammals, LPAATs have been characterized as the 1-acylglycerol-3-phophate O-acyltransferase (AGPAT) family. For example, a Homo sapiens AGPAT gene is AGPAT1. AGPAT1 localizes to the endoplasmic reticulum. In some embodiments, the amino acid sequence of AGPAT1 is SEQ ID NO: 52. In some embodiments, AGPAT1 is expressed in a cell by transformation with a plasmid comprising a sequence with at least 80%, at least 90%, at least 95%, at least 96%, at least 97%, at least 98%, at least 99%, or 100% sequence identity to SEQ ID NO: 37. Plants also often have several LPAAT genes; for example, Arabidopsis thaliana has 5 LPAAT genes. In most microalgae and higher plants, TAG biosynthesis occurs both in the chloroplast and endoplasmic reticulum (ER), and the substrate preference for LPAAT enzymes is usually oleoyl-CoA. Among other eukaryotes, however, including animals and fungi, the main organelle for TAG biosynthesis is the endoplasmic reticulum (ER), and once again, the substrate preference for LPAAT enzymes is usually oleoyl-CoA.

The enzyme having lysophosphatidic acid acyltransferase activity can comprise four catalytic motifs: (I) NHXXXXD (or NHX₄D); (II) (F/Y)XXR; (III) EGXR; and (IV) Proline, wherein X is any amino acid. Motif I can comprise a conserved NHXXXXD (or NHX₄D) sequence in which the residue following histidine (H) is usually hydrophilic, whereas the residue preceding aspartic acid (D) is almost always hydrophobic. Motif II can comprise a (F/Y)XXR sequence, in which the first residue is either phenylalanine (F) or tyrosine (Y) followed by two residues and a conserved arginine (R). The residue that precedes this R can be hydrophilic, whereas the residue that precedes this hydrophilic residue can be hydrophobic. Motif III can comprise a conserved EGXR sequence. Motif IV can comprise a conserved proline (P). An enzyme described herein can comprise a sequence with one, two, three, or four of these catalytic motifs. A cell described herein can comprise an exogenous gene comprising a sequence that encodes for an enzyme with one, two, three, or four of these catalytic motifs.

In some embodiments, an enzyme can comprise a sequence with at least 70%, at least 85%, or 100% sequence identity to any one of SEQ ID NO: 107-109. In some embodiments, a cell can comprise an exogenous gene comprising a sequence that encodes for an enzyme with at least 70%, at least 85%, or 100% sequence identity to any one of SEQ ID NO: 107-109.

In some embodiments, an enzyme can comprise a sequence with at least 60%, at least 75%, at least 85%, or 100% sequence identity to any one of SEQ ID NO: 110-119. In some embodiments, a cell can comprise an exogenous gene comprising a sequence that encodes for an enzyme with at least 60%, at least 75%, at least 85%, or 100% sequence identity to any one of SEQ ID NO: 110-119.

In some embodiments, an enzyme can comprise EGHR or EGTR. In some embodiments, an enzyme can comprise a sequence with at least 50%, at least 55%, at least 60%, at least 65%, at least 70%, at least 75%, at least 80%, at least 90%, or 100% sequence identity to any one of SEQ ID NO: 120-134. In some embodiments, a cell can comprise an exogenous gene comprising a sequence that encodes for an enzyme with at least 50%, at least 55%, at least 60%, at least 65%, at least 70%, at least 75%, at least 80%, at least 90%, or 100% sequence identity to any one of SEQ ID NO: 120-134.

In some embodiments, an enzyme described herein can comprise catalytic motif IV: a conserved proline. In some embodiments, an enzyme described herein can comprise a third domain having a sequence with a conserved proline. In some embodiments, an enzyme can comprise a sequence with at least 55%, at least 60%, at least 65%, at least 70%, at least 75%, at least 80%, at least 90%, or 100% sequence identity to any one of SEQ ID NO: 135-154. In some embodiments, a cell can comprise an exogenous gene comprising a sequence that encodes for an enzyme with at least 55%, at least 60%, at least 65%, at least 70%, at least 75%, at least 80%, at least 90%, or 100% sequence identity to any one of SEQ ID NO: 135-154.

Chlamydomonas reinhardtii, a unicellular green algae, comprises two LPAAT genes: CrLPAAT1 and CrLPAAT2. The chlorophyte specific CrLPAAT2 enzyme localizes to ER membranes. Unlike canonical ER-located LPAATs, however, CrLPAAT2 prefers palmitoyl-CoA over oleoyl-CoA as the acyl donor substrate. In some embodiments, the amino acid sequence of CrLPAAT1 is SEQ ID NO: 12. In some embodiments, CrLPAAT1 is expressed in a cell by transformation with a plasmid comprising a sequence with at least 80%, at least 90%, at least 95%, at least 96%, at least 97%, at least 98%, at least 99%, or 1⁰⁰% sequence identity to SEQ ID NO: 2. In some embodiments, the amino acid sequence of CrLPAAT2 is SEQ ID NO: 13. In some embodiments, CrLPAAT2 is expressed in cells by transformation with a plasmid comprising a sequence with at least 80%, at least 90%, at least 95%, at least 96%, at least 97%, at least 98%, at least 99%, or 100% sequence identity to SEQ ID NO: 10. Related species Chlamydomonas schloesseri, Chlamydomonas incerta, and Chlamydomonas eustigma have similar genes—ChsLPAAT2, CiLPAAT2, and CeLPAAT2, respectively. In some embodiments, the amino acid sequence of ChsLPAAT2 is SEQ ID NO: 14. In some embodiments, ChsLPAAT1 is expressed in a cell by transformation with a plasmid comprising a sequence with at least 80%, at least 90%, at least 95%, at least 96%, at least 97%, at least 98%, at least 99%, or 100% sequence identity to SEQ ID NO: 9. In some embodiments, the amino acid sequence of CiLPAAT2 is SEQ ID NO: 15. In some embodiments, CiLPAAT1 is expressed in a cell by transformation with a plasmid comprising a sequence with at least 80%, at least 90%, at least 95%, at least 96%, at least 97%, at least 98%, at least 99%, or 100% sequence identity to SEQ ID NO: 10. In some embodiments, the amino acid sequence of CeLPAAT2 is SEQ ID NO: 53. In some embodiments, CeLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 38.

The marine microalgae genus Nannochloropsis has 4 LPAAT genes. Two of these genes, NoLPAT3 and NoLPAT4, are critical for TAG biosynthesis. NoLPAT3 and NoLPAT4 are typically found around the perimeter of lipid droplets, although these two genes are unrelated to other lipid droplet-associated proteins. The location of NoLPAT3 and NoLPAT4 on the perimeter of lipid droplets enhances accumulation of TAG, particularly during nutrient deprivation. In some embodiments, the amino acid sequence of NoLPAT3 is SEQ ID NO: 16. In some embodiments, NoLPAT3 is expressed in a cell by transformation with a plasmid comprising a sequence with at least 80%, at least 90%, at least 95%, at least 96%, at least 97%, at least 98%, at least 99%, or 100% sequence identity to SEQ ID NO: 7. In some embodiments, the amino acid sequence of NoLPAT4 is SEQ ID NO 17. In some embodiments, is expressed in a cell by transformation with a plasmid comprising a sequence with at least 80%, at least 90%, at least 95%, at least 96%, at least 97%, at least 98%, at least 99%, or 100% sequence identity to SEQ ID NO: 4.

In the oilseed rape Brassica napus, one of the LPAAT genes involved in TAG biosynthesis is BnBAT2. BnBAT2 is a prokaryotic-type LPAAT gene. In some embodiments, the amino acid sequence of BnBAT2 is SEQ ID NO: 18. In some embodiments, BnBAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 3.

The cyanobacteria Synechocystis sp. LPAAT gene is referred to as Sll1848. Sll1848 incorporates C18:0 and C18:1 in the sn-2 position, and is the major LPAAT specific to C16:0 in Synechocystis sp. In some embodiments, the amino acid sequence of Sll1848 is SEQ ID NO: 19. In some embodiments, Sll1848 is expressed in a cell by transformation with a plasmid comprising a sequence with at least 80%, at least 90%, at least 95%, at least 96%, at least 97%, at least 98%, at least 99%, or 100% sequence identity to SEQ ID NO: 5.

Species of green algae such as Volvox carteri, Volvox africanus, and Volvox reticuliferus also have similar LPAAT genes—VcLPAAT2, VaLPAAT2, and VrLPAAT2, respectively. In some embodiments, the amino acid sequence of VcLPAAT2 is SEQ ID NO: 20 or SEQ ID NO: 40. In some embodiments, VcLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 8 or SEQ ID NO: 25. In some embodiments, the amino acid sequence of VaLPAAT2 is SEQ ID NO: 21. In some embodiments, VaLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 11. In some embodiments, the amino acid sequence of VrLPAAT2 is SEQ ID NO: 55. In some embodiments, VrLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 57.

The LPAAT genes in Escherichia coli are referred to as PlsC genes. In E. coli, the EcPlsC mutant strain is characterized by a deficiency of LPAAT activity. The strain is also temperature-sensitive, unable to grow at 42° C. In some embodiments, the amino acid sequence of EcPlsC is SEQ ID NO: 22. In some embodiments, EcPlsC is expressed in a cell by transformation with a plasmid comprising a sequence with at least 80%, at least 90%, at least 95%, at least 96%, at least 97%, at least 98%, at least 99%, or 100% sequence identity to SEQ ID NO: 6.

The LPAATs of the microalgae Prototheca moriformis display a strong preference for insertion of unsaturated fatty acids at the sn-2 position, rarely inserting saturates. In some embodiments, the amino acid sequence of PmLPAAT1 is SEQ ID NO: 23. In some embodiments, the amino acid sequence of PmLPAAT2 is SEQ ID NO: 24.

Species of green algae such as Chlorella sorokiniana, Chlorella variabilis, Chlorella desiccata, and Chlorella vulgaris also have similar LPAAT genes—ChsoLPAAT2, ChvaLPAAT2, ChdeLPAAT2, and ChvuLPAAT2, respectively. In some embodiments, the amino acid sequence of ChsoLPAAT2 is SEQ ID NO: 46. In some embodiments, ChsoLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 31. In some embodiments, the amino acid sequence of ChvaLPAAT2 is SEQ ID NO: 47. In some embodiments, ChvaLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 32. In some embodiments, the amino acid sequence of ChdeLPAAT2 is SEQ ID NO: 49. In some embodiments, ChdeLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 34. In some embodiments, the amino acid sequence of ChvuLPAAT2 is SEQ ID NO: 56. In some embodiments, ChvuLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 58.

The multicellular green algae Astrephomene gubernaculifera comprises a LPAAT gene referred to as AgLPAAT2. In some embodiments, the amino acid sequence of AgLPAAT2 is SEQ ID NO: 41. In some embodiments, AgLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 26.

The green algae Edaphochlamys debaryana comprises a LPAAT gene referred to as EdLPAAT2. In some embodiments, the amino acid sequence of EdLPAAT2 is SEQ ID NO: 42. In some embodiments, EdLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 27.

The unicellular green algae Dunaliella salina comprises a LPAAT gene referred to as DsLPAAT2. In some embodiments, the amino acid sequence of DsLPAAT2 is SEQ ID NO: 43. In some embodiments, DsLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 28.

The colonial green algae Scenedesmus sp. comprises a LPAAT gene referred to as SceLPAAT2. In some embodiments, the amino acid sequence of SceLPAAT2 is SEQ ID NO: 44. In some embodiments, SceLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 29.

The colonial green algae Volvulina compacta comprises a LPAAT gene referred to as VcomLPAAT2. In some embodiments, the amino acid sequence of VcomLPAAT2 is SEQ ID NO: 83. In some embodiments, VcomLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 65.

The colonial green algae Pleodorina japonica comprises a LPAAT gene referred to as PjapLPAAT2. In some embodiments, the amino acid sequence of PjapLPAAT2 is SEQ ID NO: 86. In some embodiments, PjapLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 68.

The colonial green algae Volvulina boldii comprises a LPAAT gene referred to as VbolLPAAT2. In some embodiments, the amino acid sequence of VbolLPAAT2 is SEQ ID NO: 87. In some embodiments, VbolLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 69.

The colonial green algae Gonium multicoccum comprises a LPAAT gene referred to as GmulLPAAT2. In some embodiments, the amino acid sequence of GmulLPAAT2 is SEQ ID NO: 91. In some embodiments, GmulLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 73.

The colonial green algae Gonium viridistellatum comprises a LPAAT gene referred to as GvirLPAAT2. In some embodiments, the amino acid sequence of GvirLPAAT2 is SEQ ID NO: 92. In some embodiments, GvirLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 74.

The unicellular green algae Micractinium conductrix comprises a LPAAT gene referred to as McLPAAT2. In some embodiments, the amino acid sequence of McLPAAT2 is SEQ ID NO: 45. In some embodiments, McLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 30.

The unicellular green algae Dunaliella salina comprises a LPAAT gene referred to as DsalLPAAT2. In some embodiments, the amino acid sequence of DsalLPAAT2 is SEQ ID NO: 96. In some embodiments, DsalLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 78.

The microalgae Raphidocelis subcapita comprises a LPAAT gene referred to as RsLPAAT2. In some embodiments, the amino acid sequence of RsLPAAT2 is SEQ ID NO: 48. In some embodiments, RsLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 33.

The heterotrophic green algae Auxenochlorella protothecoides comprises a LPAAT gene referred to as ApLPAAT2. In some embodiments, the amino acid sequence of ApLPAAT2 is SEQ ID NO: 50. In some embodiments, ApLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 35.

The green algae Chloropicon primus comprises a LPAAT gene referred to as ChprLPAAT2. In some embodiments, the amino acid sequence of ChprLPAAT2 is SEQ ID NO: 51. In some embodiments, ChprLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 36.

The green algae class Pedinophyceae comprises a LPAAT gene referred to as PedLPAAT2. In some embodiments, the amino acid sequence of PedLPAAT2 is SEQ ID NO: 54. In some embodiments, PedLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 39. In some embodiments, PedLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 59. In some embodiments, PedLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 60. In some embodiments, PedLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 61. In some embodiments, PedLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 62.

The green algae Vitreochlamys sp. CL-2021 comprises a LPAAT gene referred to as VitrLPAAT2. In some embodiments, the amino acid sequence of VitrLPAAT2 is SEQ ID NO: 84. In some embodiments, VitrLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 66.

The green algae Colemanosphaera charkowiensis comprises a LPAAT gene referred to as CchaLPAAT2. In some embodiments, the amino acid sequence of CchaLPAAT2 is SEQ ID NO: 85. In some embodiments, CchaLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 67.

The green algae Volvulina boldii comprises a LPAAT gene referred to as VbolLPAAT2. In some embodiments, the amino acid sequence of VbolLPAAT2 is SEQ ID NO: 87. In some embodiments, VbolLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 69.

The green algae Pandorina morum comprises a LPAAT gene referred to as PmorLPAAT2. In some embodiments, the amino acid sequence of PmorLPAAT2 is SEQ ID NO: 88. In some embodiments, PmorLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 70.

The green algae Volvox carteri f. weismannia comprises a LPAAT gene referred to as VcarfLPAAT2. In some embodiments, the amino acid sequence of VcarfLPAAT2 is SEQ ID NO: 89. In some embodiments, VcarfLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 71.

The green algae Eudorina cylindrica comprises a LPAAT gene referred to as EcylLPAAT2. In some embodiments, the amino acid sequence of EcylLPAAT2 is SEQ ID NO: 90. In some embodiments, EcylLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 72.

The green algae Volvox ferrisii comprises a LPAAT gene referred to as VferLPAAT2. In some embodiments, the amino acid sequence of VferLPAAT2 is SEQ ID NO: 93. In some embodiments, VferLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 75.

The green algae Vitreochlamys aulata comprises a LPAAT gene referred to as VaulLPAAT2. In some embodiments, the amino acid sequence of VaulLPAAT2 is SEQ ID NO: 94. In some embodiments, VaulLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 76.

The green algae Chlamydomonas sp. CCAC2762_B comprises a LPAAT gene referred to as Ch_CCAC2762_LPAAT2. In some embodiments, the amino acid sequence of Ch_CCAC2762_LPAAT2 is SEQ ID NO: 95. In some embodiments, Ch_CCAC2762_LPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 77.

The green algae Microglena sp. YARC comprises a LPAAT gene referred to as MyarcLPAAT2. In some embodiments, the amino acid sequence of MyarcLPAAT2 is SEQ ID NO: 97. In some embodiments, MyarcLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 79.

The green algae Chlamydomonas sp. UWO_241 comprises a LPAAT gene referred to as CuwoLPAAT2. In some embodiments, the amino acid sequence of CuwoLPAAT2 is SEQ ID NO: 98. In some embodiments, CuwoLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 80.

The green algae Chlamydomonas moewusii comprises a LPAAT gene referred to as CmoeLPAAT2. In some embodiments, the amino acid sequence of CmoeLPAAT2 is SEQ ID NO: 99. In some embodiments, CmoeLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 81.

The green algae Oophila amblystomatis comprises a LPAAT gene referred to as OambLPAAT2. In some embodiments, the amino acid sequence of OambLPAAT2 is SEQ ID NO: 100. In some embodiments, OambLPAAT2 is expressed in a cell by transformation with a plasmid comprising a sequence with at least 80%, at least 90%, at least 95%, at least 96%, at least 97%, at least 98%, at least 99%, or 100% sequence identity to SEQ ID NO: 82.

The green algae Edaphochlamys debaryana_SRR13719274 comprises a LPAAT gene referred to as EdLPAAT2. In some embodiments, the amino acid sequence of EdLPAAT2 (Edaphochlamys debaryana_SRR13719274) is SEQ ID NO: 101.

The colonial green algae Volvulina steinii comprises a LPAAT gene referred to as VsteLPAAT2. In some embodiments, the amino acid sequence of VsteLPAAT2 is SEQ ID NO: 102.

The colonial green algae Gonium octonarium comprises a LPAAT gene referred to as GoctLPAAT2. In some embodiments, the amino acid sequence of GoctLPAAT2 is SEQ ID NO: 103.

The unicellular green algae Chlamydomonas nivalis comprises a LPAAT gene referred to as CnivLPAAT2. In some embodiments, the amino acid sequence of CnivLPAAT2 is SEQ ID NO: 104.

The green algae Pycnococcus provasolii comprises a LPAAT gene referred to as PproLPAAT2. In some embodiments, the amino acid sequence of PproLPAAT2 is SEQ ID NO: 105.

The green algae Bathycoccus prasinos comprises a LPAAT gene referred to as BpraLPAAT2. In some embodiments, the amino acid sequence of GoctLPAAT2 is SEQ ID NO: 106.

CrLPAAT1-ΔCST* is a Chlamydomonas reinhardtii LPAAT with a total of 30 amino acids (aa 12-41; CST signal) removed from CrLPAAT1. BnBAT2-ΔCST* is a Brassica napus LPAAT with a total of 86 amino acids (aa 2-87) removed from BnBAT2. In some embodiments, the amino acid sequence of CrLPAAT1-ΔCST* is SEQ ID NO: 155. In some embodiments, the amino acid sequence of BnBAT2-ΔCST* is SEQ ID NO: 156.

### Tag Oils of the Disclosure

Provided herein are TAG oil compositions having TAG species with a saturated fatty acid at the sn-2 position. In some embodiments, the TAG species is OPO.

The complexity and physical properties of an oil can be evaluated by the fatty acid profile of the TAG component of an oil and the TAG profile. The fatty acid profile is a measure of fatty acid composition and can be determined by subjecting an oil to transesterification to generate fatty acid methyl esters (FAMEs) and subsequently quantitating fatty acid type by Gas Chromatography equipped with a Flame Ionization Detector (GC/FID). Accordingly, fatty acid content of a TAG oil provided herein can be determined by GC/FID. Since TAGs comprise three fatty acids arrayed along the glycerol backbone in the triglyceride molecule, the number of possible distinct regioisomers of TAGs can be defined by the number of fatty acid species in the oil raised to the third power.

Genetic expression of an enzyme described herein can be modulated to enrich for OPO production. Expression of a gene described herein can be modulated singly or in combination with expression of one or more enzymes described herein, to enrich for OPO production. Genetic expression of an enzyme described herein can be modulated to enhance LPAAT activity. Expression of a gene described herein can be modulated singly or in combination with expression of one or more enzymes described herein, to enhance LPAAT activity. In some embodiments, a cell described herein is modified to express an exogenous gene to enhance LPAAT activity. In some embodiments, a cell described herein is modified to delete an endogenous gene to enhance LPAAT activity.

Provided herein are methods of genetically modifying and cultivating a cell to enrich for production of an oil provided herein. Further provided herein are cells that are genetically modified to enrich for production of an oil described herein. A cell provided herein can contain recombinant nucleic acids operable to increase expression of a gene described herein to enhance LPAAT activity. Further, a cell provided herein can exclude endogenous nucleic acids operable to decrease expression of a gene described herein to enhance LPAAT activity. In some embodiments, a microalgal cell is modified to enrich for production of an oil provided herein.

Oil compositions provided herein are produced by a microorganism, such as a microalgal cell. As such, an oil composition produced therefrom may comprise one or more sterol components that are characteristic of an algal oil. The amount of a sterol component in an oil can be determined on a weight-by-weight basis, e.g., mg of a sterol per 100 g of the oil. Sterol composition (i.e., a sterol profile) can be determined by mass spectrometry, for example, gas chromatography-mass spectrometry (GC-MS); liquid chromatography-mass spectrometry (LC-MS); tandem mass spectrometry (MS/MS), and coupled liquid and gas chromatography with subsequent flame ionization detection (LC-GC-FID). An example sterol profile as determined by GC/MS analysis of TMS esters of the non-saponifiable fraction of the RBD oil produced from CHK22 is shown in Table 26. Cholestan-3-ol(3a,5b) was used as an internal standard (ISTD).

TABLE 26
CHK22 RBD Oil Sterol Profile

| Sterol Name | mg/100 g | % w/w |
|---|---|---|
| Cholestan-3-ol (3a,5b) ISTD | ** ISTD ** | ** ISTD ** |
| Ergosta-7,22-dien-3-ol, | 9.288 | 0.009 |
| (3β,22E)-, TMS derivative | | |
| Unknown 2 | 107.521 | 0.108 |
| Ergosterol, TMS derivative | 124.381 | 0.124 |
| Unknown | 69.057 | 0.069 |
| Unknown | 35.660 | 0.036 |
| 5.Xi.-Ergost-7-en-3β-ol, | 120.417 | 0.120 |
| TMS derivative | | |
| Unknown | 10.601 | 0.011 |
| 9,19-Cyclolanostan-3-ol, | 5.366 | 0.005 |
| 24-methylene-, (3β)-, O-TMS | | |

In some embodiments, the oil contains a positive amount of ergosta-7,22-dien-3-ol, (3β,22E)-. In some embodiments, the amount of ergosta-7,22-dien-3-ol, (3β,22E)- in an oil provided herein is at least 2 mg, at least 5 mg, or at least 8 mg, or at least 9 mg per 100 g of the oil. In some embodiments, the amount of ergosta-7,22-dien-3-ol, (3β,22E)- in an oil is 1-100 mg, 2-30 mg, 3-20 mg, or 5-15 mg per 100 g of the oil.

In some embodiments, the oil contains a positive amount of ergosterol. In some embodiments, the amount of ergosterol in an oil provided herein is at least 50 mg, at least 100 mg, or at least 125 mg per 100 g of the oil. In some embodiments, the amount of ergosterol in an oil provided herein is at least 10 mg, at least 20 mg, at least 30 mg, at least 40 mg, at least 50 mg, at least 60 mg, at least 70 mg, at least 80 mg, at least 90 mg, at least 100 mg, at least 110 mg, at least 120 mg, at least 130 mg, at least 140 mg, at least 150 mg, at least 160 mg, at least 170 mg, at least 180 mg, at least 190 mg, or at least 200 mg per 100 g of the oil. In some embodiments, the amount of ergosterol in an oil is 10-50 mg, 50-100 mg, 100-150 mg, 150-200 mg, 10-100 mg, 100-200 mg, 100-2000 mg, or 10-200 mg per 100 g of the oil.

In some embodiments, the oil contains a positive amount of campesterol, a positive amount of β-sitosterol, or a positive amount of stigmasterol. In some embodiments, the amount of campesterol, β-sitosterol, or stigmasterol is no more than 5 mg per 100 g of the oil. In some embodiments, the oil does not contain campesterol. In some embodiments, the amount of campesterol in an oil provided herein is no more than 5 mg of campesterol per 100 g of the oil. In some embodiments, the oil does not contain stigmasterol. In some embodiments, the amount of stigmasterol in an oil is no more than 5 mg per 100 g of the oil. In some embodiments, the oil does not contain β-sitosterol. In some embodiments, the amount of β-sitosterol in an oil is no more than 5 mg per 100 g of the oil.

In some embodiments, the oil contains a positive amount of ergosta-5,8-dien-3-ol, (3β)-. In some embodiments, the amount of ergosta-5,8-dien-3-ol, (3β)- is at least 1 mg per 100 g of the oil. In some embodiments, the amount of ergosta-5,8-dien-3-ol, (3β)- is at least 1 mg, at least 2 mg, at least 3 mg, at least 4 mg, at least 5 mg, at least 6 mg, at least 7 mg, at least 8 mg, at least 9 mg, at least 10 mg, at least 15 mg, at least 20 mg, at least 25 mg, at least 30 mg, at least 35 mg, at least 40 mg, at least 45 mg, or at least 50 mg per 100 g of the oil. In some embodiments, the amount of ergosta-5,8-dien-3-ol, (3β)- is 1-5 mg, 10-20 mg, 50-100 mg, 100-150 mg, 150-200 mg, 1-50 mg, 20-100 mg, 100-200 mg, or 20-200 mg per 100 g of the oil.

In some embodiments, the oil contains a positive amount of 5.xi.-ergost-7-en-3β-ol. In some embodiments, the amount of 5.xi.-ergost-7-en-30-ol is at least 15 mg per 100 g of the oil. In some embodiments the amount of 5.xi.-ergost-7-en-30-ol is at least 20 mg per 100 g of the oil. In some embodiments, the amount of 5.xi.-ergost-7-en-30-ol is at least 1 mg, at least 2 mg, at least 3 mg, at least 4 mg, at least 5 mg, at least 10 mg, at least 15 mg, at least 20 mg, at least 25 mg, at least 30 mg, at least 35 mg, at least 40 mg, at least 45 mg, at least 50 mg, at least 60 mg, at least 70 mg, at least 80 mg, at least 90 mg, at least 100 mg, at least 110 mg, at least 120 mg, at least 130 mg, at least 140 mg, at least 150 mg, at least 160 mg, at least 170 mg, at least 180 mg, at least 190 mg, or at least 200 mg per 100 g of the oil. In some embodiments, the amount of 5.xi.-ergost-7-en-30-ol is 1-50 mg, 20-50 mg, 50-100 mg, 100-150 mg, 150-200 mg, 20-100 mg, 100-2000 mg, or 20-200 mg per 100 g of the oil.

In some embodiments, the oil contains a positive amount of 9,19-cyclolanostan-3-ol,24-methylene-,(3β)-. In some embodiments, the amount of 9,19-cyclolanostan-3-ol,24-methylene-,(3β)- is at least 5 mg of per 100 g of the oil. In some embodiments, the amount of 9,19-cyclolanostan-3-ol,24-methylene-,(3β)- is at least 1 mg, at least 2 mg, at least 3 mg, at least 4 mg, at least 5 mg, at least 10 mg, at least 15 mg, at least 20 mg, at least 25 mg, at least 30 mg, at least 35 mg, at least 40 mg, at least 45 mg, or at least 50 mg per 100 g of the oil. In some embodiments, the amount of 9,19-cyclolanostan-3-ol,24-methylene-,(3β)- is 5-10 mg, 10-20 mg, 20-30 mg, 30-40 mg, 40-50 mg, 5-25 mg, 25-50 mg, or 5-50 mg of per 100 g of the oil.

TAG profile analysis of oil compositions provided herein can be conducted using liquid chromatography-mass spectrometry (LC-MS). TAG oils provided herein can be extracted from cells into a solution of 3:1 toluene/2-propanol (v/v) by means of mechanical disruption using ceramic beads and vigorous agitation. Filtered extracts can then be injected on LC mass spectrometer for profiling.

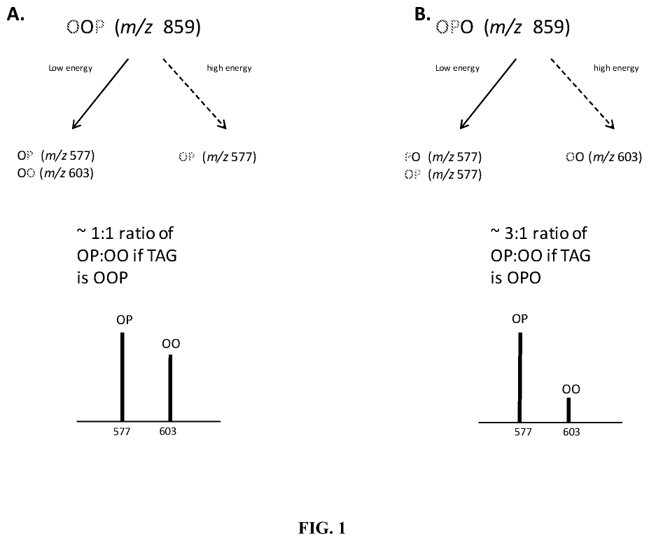

Chromatographic separation of TAG regioisomers cannot be achieved by the LCMS method described. Instead, qualitative assessment of the regiospecificity of a predominant TAG species can be performed based on the abundance of diacylglycerol (DAG) ions resulting from fragmentation of the TAG species (FIG. 1). In LC-APCI-MS analyses of TAGs, the loss of the fatty acid at the sn-1 and sn-3 positions are energetically more favorable compared to the sn-2 position, which results in a greater abundance of the DAG ions formed from those fragments. In the case of an OOP regioisomer, an approximate 1:1 ratio of the OP DAG ion (m/z 577) and OO DAG ion (m/z 603) can be observed, as shown in FIG. 1, Panel A. In the case of the OPO regioisomer, a greater abundance of the OP DAG ion (m/z 577) can be observed, as shown in FIG. 1, Panel B.

FIG. 1 demonstrates that TAG species of the same molecular weight show distinctive daughter ion ratios. The two TAG species, OOP and OPO in Panel A and Panel B, respectively, have identical mass/charge (m/z) ratios for the parent ion (859), but as illustrated, the molecules fragment very differently due to the effects of differing collision energies. Low energy collisions eliminate fatty acyl groups from the sn-1 or sn-3 position with greater frequency due to the relative inaccessibility of the acyl group at the sn-2 position. In the case of OOP, as shown in Panel A, low energy collisions result in daughter ions with m/z of 577 (OP) and 603 (OO), while less frequent high energy collisions resulting in the elimination of O from the sn-2 position also results in a daughter ion of m/z 577, OP. As a consequence, the resulting mass spectra of the daughter ions is the sum of all these fragments. The peak at 577 (OP) results from the more frequent elimination of O at sn-1 and the less frequent elimination of O at sn-2. Hence, the 577 fragment is roughly 1.5-2× the height of the 603 fragment (OO) resulting from the more frequent elimination of P at the sn-3 position. Alternatively, and as shown in Panel B, the OPO TAG species shows very different daughter ion ratios as the products of low energy collisions PO and OP have the same m/z of 577, while the daughter ion of the less frequently generated, high energy collision representing OO has a m/z of 603. For this TAG species, the 577 peak height, resulting from the summation of the low energy derived PO and OP daughter ions, is substantially greater than that for the 603 fragment, which requires much higher energy to generate.

In some embodiments, the oil can have an OP:OO m/z ratio of as determined by abundance of DAG ions resulting from mass spectrometry fragmentation of the TAG component in the oil. In some embodiments, the oil can have an OP:OO m/z ratio of at least 1.6 as determined by abundance of DAG ions resulting from mass spectrometry fragmentation of the TAG component in the oil. In some embodiments, the oil can have an OP:OO m/z ratio of at least 1.6, at least 1.7, at least 1.8, at least 1.9, at least 2.0, at least 2.1, at least 2.2, at least 2.3 at least 2.4 at least 2.5, at least 2.6, at least 2.7, at least 2.8, at least 2.9, at least 3.0, at least 3.1, at least 3.2, at least 3.3, at least 3.4, at least 3.5, at least 3.6, at least 3.6, at least 3.7, at least 3.8, at least 3.9, at least 4.0, at least 4.1, at least 4.2, at least 4.3, at least 4.4, at least 4.5, at least 4.6, at least 4.7, at least 4.8, at least 4.9, or at least 5.0 as determined by abundance of DAG ions resulting from mass spectrometry fragmentation of the TAG component in the oil.

TAG regiospecificity can further be determined by a porcine pancreatic lipase assay. This method can be used to determine identity of the sn-2 fatty acid of a TAG by incubating the TAG with porcine pancreatic lipase. The lipase reaction results in deacylation of the fatty acids at the sn-1 and sn-3 positions. The remaining sn-2-monoacylglycerols (MAGs) can then be isolated and identified by solid phase extraction and fatty acid composition analysis by gas chromatography using a direct transesterification method.

Oil compositions provided herein can include a TAG component in which at least 40% of the TAGs in the TAG component are TAG species having a saturated fatty acid at the sn-2 position. In some embodiments, at least about 50% of the TAGs in the TAG component is a TAG species having a saturated fatty acid at the sn-2 position. In some embodiments, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% of the TAGs in the TAG component is a TAG species having a saturated fatty acid at the sn-2 position. In some embodiments, 40-45%, 45-50%, 50-55%, 55-60%, 40-50%, 50-60%, or 40-60% of the TAGs in the TAG component is TAG species having a saturated fatty acid at the sn-2 position. In some embodiments, the saturated fatty acid at the sn-2 position is palmitate.

Oil compositions provided herein can include a TAG component in which at least some of the acyl chains of the TAG component are C18:1. In some embodiments, at least about 50% of the acyl chains in the TAG component are C18:1. In some embodiments, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, or at least about 70% of the acyl chains in the TAG component are C18:1. In some embodiments, at least about 50-55%, at least about 55-60%, at least about 60-65%, at least about 60-67%, at least about 65-70%, at least about 50-60%, at least about 50-67%, at least about 60-70%, or at least about 50-70% of the acyl chains in the TAG component are C18:1.

### Microbial Oils

In some embodiments, an oil provided herein is obtained from a genetically modified microorganism, for example, microalgae, oleaginous yeast, or oleaginous bacteria. In some embodiments, the genetically modified microorganism is a genetically modified Prototheca sp. strain. In some embodiments, a genetically modified microorganism comprises an exogenous gene or exogenous nucleotides. Alternatively, or additionally, a genetically modified microorganism does not comprise an endogenous gene or endogenous nucleotides. Genetic modification methods described herein can be used to confer enriched oleic acid and/or OPO TAG production in a microorganism.

An oil provided herein can be obtained from microalgae, oleaginous yeast, or oleaginous bacteria. In some embodiments, the microorganism is a modified Prototheca sp. strain. A non-genetically modified Prototheca sp. strain can be produced by classical strain improvement strategies. A genetically modified Prototheca sp. strain can be produced by expression of an exogenous gene and/or deletion of an endogenous gene in the microorganism. In some embodiments, a microorganism provided herein is a genetically modified Prototheca sp. strain produced by classical strain improvement strategies to optimize for enriched oleic acid production (e.g., at least 50% oleic acid) and enriched OPO production (e.g., at least 70% OPO). In some embodiments, a microorganism provided herein is a genetically modified Prototheca sp. strain produced by classical strain improvement strategies to optimize for enriched palmitic acid production (e.g., at least 30% palmitic acid) and enriched OPO production (e.g., at least 70% OPO).

An oil provided herein can be produced from a Prototheca cell. The oil produced can be from a UTEX 1533 Prototheca strain cell. The oil produced can be from a cell of a strain that does not express an exogenous thioesterase.

In some embodiments, an oil provided herein is produced by microalgae. In some embodiments, the microalgae is a species of a genus selected from the group consisting of: Chlorella sp., Pseudochlorella sp., Heterochlorella sp., Prototheca sp., Arthrospira sp., Euglena sp., Nannochloropsis sp., Phaeodactylum sp., Chlamydomonas sp., Scenedesmus sp., Ostreococcus sp., Selenastrum sp., Haematococcus sp., Nitzschia, Dunaliella, Navicula sp., Trebouxia sp., Pseudotrebouxia sp., Vavicula sp., Bracteococcus sp., Gomphonema sp., Watanabea, sp., Botryococcus sp., Tetraselmis sp., and Isochrysis sp. In some embodiments, the microalgae is Prototheca sp. In some embodiments, the microalgae is P. mornformis. In some embodiments, the microalgae is P. wickerhamii.

### Genetic Engineering and Classical Strain Improvement

Production of TAGs oils having particular phenotypes can be achieved by genetic and non-genetic modification techniques of microorganisms. While genetic engineering techniques can be used to targeted to phenotypes elicited in a host oleaginous microbe, classical strain improvement and other non-genetic engineering techniques can be employed to further enhance these phenotypes. Similarly, classical strain improvement or other non-genetic engineering techniques can be employed to enhance certain phenotypes that have been selected by genetic engineering techniques. Phenotypes can include elaboration of a particular fatty acid profile, a particular TAG profile, yield on carbon, growth productivity, volumetric oil accumulation (e.g., g oil/L culture), oil productivity (e.g., g oil/L culture day), and oil as a percent dry cell weight (DCW) as a measure of strain performance.

In some embodiments, a microalgal cell provided herein produces at least 50% lipid by dry cell weight. In some embodiments, a microalgal cell provided herein produces at least 60% lipid by dry cell weight.

For example, a microorganism that has been non-genetically modified to confer a first phenotype (e.g., enriched oleic acid production) can be genetically modified to confer a second phenotype (e.g., enriched OPO production). The resulting microorganism can then confer both the first and second phenotype (e.g., enriched oleic acid and OPO production).

Microorganisms provided herein can be produced by classical strain improvement strategies to select for microorganism having a desired phenotype, e.g., high oleic oil production. Classical strain improvement (also called “mutation breeding”) involves exposing organisms to chemicals or radiation to generate mutants with desirable traits. Ultraviolet (UV) light can be used to introduce random mutations within a microorganism's nuclear genome. Chemical mutagens include compounds which inhibit or disrupt biosynthetic processes of a microorganism, e.g., antibiotics, antifungals, or carcinogens. Non-limiting examples of chemical mutagens include ICR-191, ethyl methanesulfonate (EMS), and 4-nitroquinoline-1-oxide (4-NQO). Non-limiting examples of chemical mutagens also include acridine mutagens, amino acid analogs, fatty acid biosynthesis inhibitors, cholesterol biosynthetic inhibitors, mTOR inhibitors, and membrane solubilizing agents. Combinations of chemical mutagens can also be used simultaneously to induce mutagenesis. Following mutagenesis, selective or enrichment agents can be used to select or enrich for strains of interest. Non-limiting examples of enrichment agents include L-canavanine, cerulenin, triparanol, clomiphene, clomiphene citrate, clotrimazole, terfenadine, fluphenazine, AZD8055, BASF 13-338, cafenstrole, clomiphene, PF-042110, and phenethyl alcohol.

Microorganisms provided herein having enhanced or altered lysophosphatidic acid acyltransferase activity can be produced by genetic engineering techniques, such as genetic recombination. Enhancement of lysophosphatidic acid acyltransferase activity can be achieved by enhancing expression of an exogenous gene and/or reducing expression of an endogenous gene in the microorganism. Enhancement of gene expression can be through overexpression of an endogenous gene in a microorganism or expression of an exogenous gene. Reduction of gene expression can be through deletion or removal of an endogenous gene in a microorganism.

For example, a non-naturally occurring microorganism provided herein can be genetically modified to enhance or alter lysophosphatidic acid acyltransferase activity in the microorganism. Enhancement or alteration of lysophosphatidic acid acyltransferase activity can be achieved by enhancing expression of an exogenous LPAAT gene and/or reducing expression of an endogenous LPAAT gene in the microorganism.

Methods provided herein include classical strain improvement and/or genetic engineering methods to improve strain productivity, carbon yield, oleic acid content, and OPO content. Glucose consumption rate can be highly predictive indicator of lipid titer. As such, glucose consumption rate can be used as an enrichment tool in the mutant selection process.

### Polyol Applications and End Products

Oils described herein can be used as substrates in chemistries used to produce polyols, for example hydroformylation/reduction or epoxidation and ring opening. Oleic acid moieties at the sn-1 and sn-3 position, when subjected to the aforementioned chemistries, will result in diols, that, when formulated with one or more excipients for a variety of applications, including but not limited to, process oils (e.g., for tires), waxes, lubricants, other polyols, macrodiols, other polyesterdiols, can be used to create polyurethane products, e.g., hard foams, soft foams, cast polyurethanes, thermoplastic polyurethanes (TPUs), elastomers, adhesives, coatings, laminates, films, and dispersions. Polyurethane products can be used to construct aerospace, automotive, medical, electronic, building and construction goods; sporting goods or recreational equipment, e.g., skis, snowboards, sidewalls, boating equipment, kayaks; and other consumer goods, e.g., industrial containers, coolers, mattresses, leather goods, apparel, footwear, mannequins, and phone cases. These polyurethane applications can serve as sustainable alternatives to petroleum-based, non-renewable materials, such as acrylonitrile butadiene styrene (ABS), ultra-high molecular weight polyethylene (UHMWPE), or high density polyethylene (HDPE).

Oils provided herein can have improved production efficiency and a TAG composition that is enhanced for improved control of urethane chemistry. These characteristics of microbial oil can result in a greater degree of hydroxyl group (—OH) uniformity relative to oils with greater TAG heterogeneity (hence, lower purity) and/or diversity (e.g., oilseed or plant derived oils). Polyols derived from oils provided herein highly enriched in single, hydroxylated TAG species, can be preferable in generating polymers, including in instances where physical properties of a polymer can be compromised by molecular impurities, such as non-hydroxylated fatty acids or randomness in the regioselective insertion of fatty acid moieties on the glycerol backbone that may be present in oils having a more diverse or heterogeneous TAG profile.

Polyols described herein can be particularly useful for producing polyurethane materials. For example, oils provided herein can have relatively low TAG diversity, low fatty acid diversity, and the fatty acids present in the oils may be hydroxylated fatty acids. A higher ratio of hydroxylated fatty acids to non-hydroxylated fatty acids can allow for increased chemical reactivity. Oils having low TAG diversity and a high proportion of hydroxylated fatty acids can be especially desirable in production of polyurethanes because hydroxylated fatty acids that can participate in crosslinking reactions with isocyanates. Thus, polyols generated from highly hydroxylated fatty acids comprising the oil, can yield polyurethane materials having superior properties.

### Polyol Production

The hydroxyl group functionality of polyols can be introduced via a chemical conversion of a triglyceride oil. This conversion typically involves the presence of a double bond on an acyl moiety of the fatty acid, which can then be converted to incorporate one or more hydroxyl groups using several different chemistries including, for example, epoxidation/ring opening, ozonolysis, and hydroformylation and reduction.

Epoxidation and subsequent ring opening across the carbon-carbon double bonds of an acyl chain can be carried out using a variety of reagents including, for example, water, hydrogen, methanol, ethanol, propanol, isopropanol, or polyols. Ring opening can be facilitated by reaction with an alcohol, including, for example, β-substituted alcohols. Ring opening of epoxidized oil can also be effected through the use of a hydrogenation catalyst, such as nickel, to create a hydroxyl moiety.

Hydroformylation with synthesis gas (syngas) can be carried out using rhodium or cobalt catalysts to form the aldehyde at the olefinic group. The aldehyde can subsequently undergo reduction to an alcohol in the presence of hydrogen and a nickel catalyst to generate the polyol.

The hydroformylation chemistry results in the preservation of fatty acid length and formation of primary hydroxyl group moieties. Primary hydroxyl group functionalities can be desirable in some PU applications due to increased reactivity compared to secondary hydroxyl group moieties. Hydroxyl groups introduced to olefinic groups in the acyl chain can participate in subsequent downstream chemistries, i.e., reaction with an isocyanate moiety to form a urethane linkage or reaction with methyl esters to form polyesters. Saturated fatty acids which do not contain double bonds cannot participate in crosslinking reactions with isocyanates. Hence, saturated fatty acids can compromise the structural integrity and degrade performance of the polymer produced therefrom.

In some embodiments, a polyol provided herein contains a substantial proportion of primary hydroxyl groups. In some embodiments, a polyol provided herein contains secondary hydroxyl groups. In some embodiments, a polyol provided herein can be modified to increase the proportion of primary hydroxyl groups.

Derivatives of TAG oils provided herein can be starting materials for producing polyols. Non-limiting examples of these TAG derivatives include fatty acids, fatty acid methyl esters, fatty acid ethyl esters, hydroxylated fatty acids, hydroxylated fatty methyl esters, and hydroxylated fatty ethyl esters. Non-limiting examples of polyols include polyester diols, polyether diols, hydrogenated polyols, hydroformylated polyols, and epoxidized, ring opened polyols. Alternatively, TAG oils provided herein, without further chemical modification, can be directly used as starting materials for generating polyols.

Fatty acid methyl esters (FAMEs) can be generated through ester chemistry. For example, the TAG can be cleaved through transesterification into FAMEs and glycerol. In turn, FAMEs can be subjected to epoxidation and ring opening, for example, to create FAMEs of alcohols. Alternatively, polyols can first be generated from a TAG through epoxidation and ring opening, for example, followed by transesterification, into FAMEs of alcohols and glycerol. Glycerol and potassium methoxide catalyst can be removed by washing with water.

Catalysts, including potassium methoxide (KOCH₃), 1,5,7-triazabicyclo[4.4.0]dec-5-ene (TBD), Titanium(IV) isopropoxide (TIP), dibutyltin dilaurate (DBTDL), tris(pentafluorophenyl)borane (BCF), and potassium tert-butoxide, among others, can be utilized to re-esterify ester groups to alcohol moieties. The dual functionality of alcohol FAMEs can be used to create polymer networks using only the methyl esters of the alcohol. Due to the polarity of the molecules (ester on one end and alcohol at the other end), the resulting polymer networks can elongate in a linear, unidirectional manner, and terminate in a single hydroxyl group.

Polymer networks can also be elongated in a bi-directional manner by incorporation of a diol, such as low molecular weight diols or the polyols provided herein. Non-limiting examples of diols include propylene glycol, alkyl diols, 1,4-butanediol, 1,3-propanediol, and 1,6-hexanediol. In some embodiments, diols can be produced using microbial hosts.

Hyperbranched polyols can be prepared to achieve a range of properties, such as molecular weight, viscosity, branching, and reactivity. For example, hyperbranched polyols can combine with isocyanates, ionogenic molecules, or hydrophobic compounds to produce higher order polyols.

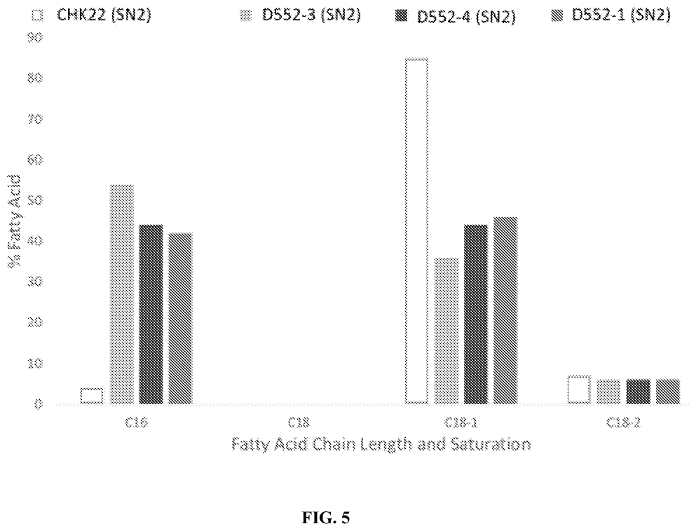

In some embodiments, TAG oils described herein can be utilized to create materials for polymer applications. As shown in FIG. 7, TAG oils enriched in OPO can undergo chemical conversion to create oils containing functional groups, such as epoxy, ethoxy, methoxy, or hydroxy. Possible modifications of OPO include (from left to right): hydroformylation and reduction, resulting in a diol; epoxidation, resulting in the di-epoxy; and epoxidation followed by ring opening (e.g., with ethanol), resulting in a diol. TAGs enriched in OPO can allow for a variety of chemistries to be deployed around the carbon-carbon double bond of the oleic moieties, which can thereby produce numerous varieties of monomers for use as chain extenders in polymer chemistry applications.

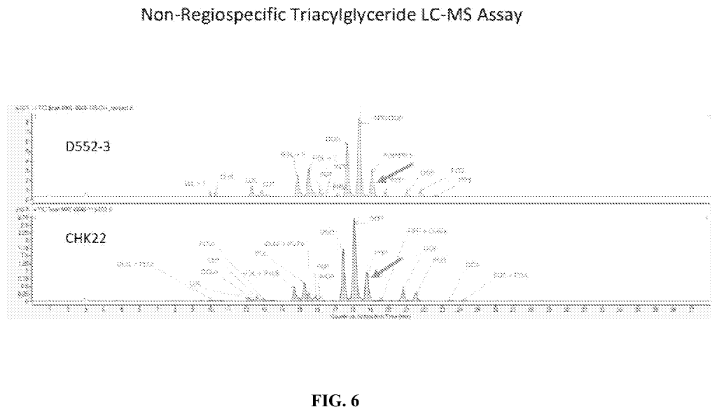

In some embodiments, strains producing OPO enriched TAGs such as those described herein can be useful substrates for additional genetic modifications with a delta-12 fatty acid desaturase (FAH12). Upregulation of FAH12 expression in such an OPO accumulating strain, can increase ricinoleic acid production, thereby increasing levels of ricinoleic acid incorporated into TAGs containing oleic acid. As a consequence, such strains can thereby produce diols, similar to those described in FIG. 7, but in vivo without requiring additional chemical modifications. Non-limiting examples of oleate 12-hydroxylases include the Lesquerella fendleri FAH12, Ricinus communis FAH12, or Claviceps purpurea FAH12. An example of such a diol is shown in FIG. 8.

### EXAMPLES

### Example 1: Expressing Chlamydomonas reinhardtii (CrLPAAT2) in P. Moriformis Strain CHK22

The microalgae Prototheca moriformis strain CHK22 (UTEX 1533) obtained from University of Texas at Austin Culture Collection of Algae produces a fatty acid ratio of oleate:palmitate of roughly 2:1, which can be suitable for OPO production. However, the P. moriformis lysophosphatidic acid acyltransferases (LPAATs) exhibit a strong preference for insertion of unsaturated fatty acids over saturated fatty acids at the sn-2 position. In this example, heterologous LPAAT genes were tested to identify genes that can efficiently direct palmitate (a saturated fatty acid) to the s-2 position of TAGs produced by the microalgae.

The C. reinhardtii CrLPAAT2 was introduced into a P. moriformis strain CHK22. The expression construct pCHK385 contained 5Y and 3Y homology arms to permit targeted integration of CrLPAAT2 into the genome and is shown in Table 1.

TABLE 1
Integrative sequences for the transformation of P. moriformis with pCHK385
encoding CrLPAAT2.

| SEQ ID | |
|---|---|
| NO: | Sequence |
| 1 | gctcttccgcttgcccgcaccctcgttgatctgggagccctgcgcagccccttaaatcatctcagtcaggtttctatgttcaactgagcc |
| | taaagggctttcgtcacgcgcacgagcacacgtatatcggctacgcagtctctcagaagcggtagaacagttcgcaagccctcgtc |
| | ggtcgaaaacttgcgccagtactattgaattaaattaaatgatcgaatgagacgcgaaacttttgcagaatgccactgagtttgccc |
| | agagaatgggagtggcgccattcaccatccgcctgtgcacggcctgattcgccgagacgatgaatggcgagaccagggagcggct |
| | tgcgagccccgagccggtagcaggaataacggtcgacaatcttcctgtccaattactggcaaccattagaaagagccggagcgcg |
| | ttgaaattctgcaatcgagtaatttttcgatgcggcgggcctgctgaaccctaaggctccggcctatgtttaaggcgatccaagatgc |
| | acgcggccccaggcacgtgtctcaagcacaaaccccagccttagtttcgagactttgggagatagcggcagatatctagtttggcat |
| | tttgtatattaattaagtctcgcaatggagcgctctgatgcggtgcagcgtcggctgcagcacctggcagtggcgctggggtcgccct |
| | 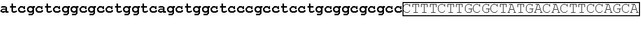 |
| | 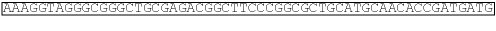 |
| | 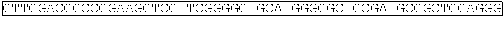 |
| | 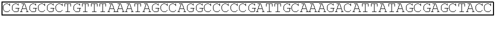 |
| | 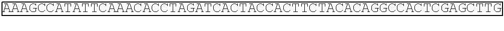 |
| | 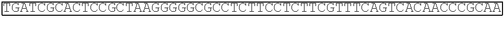 |
| | 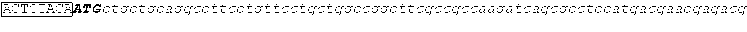 |
| | tccgaccgccccctggtgcacttcacccccaacaagggctggatgaacgaccccaacggcctgtggtacgacgagaaggacgccaa |
| | gtggcacctgtacttccagtacaacccgaacgacaccgtctgggggacgcccttgttctggggccacgccacgtccgacgacctgacc |
| | aactgggaggaccagcccatcgccatcgccccgaagcgcaacgactccggcgccttctccggctccatggtggtggactacaacaac |
| | acctccggcttcttcaacgacaccatcgacccgcgccagcgctgcgtggccatctggacctacaacaccccggagtccgaggagcag |
| | tacatctcctacagcctggacggcggctacaccttcaccgagtaccagaagaaccccgtgctggccgccaactccacccagttccgcg |
| | acccgaaggtcttctggtacgagccctcccagaagtggatcatgaccgcggccaagtcccaggactacaagatcgagatctactcctc |
| | cgacgacctgaagtcctggaagctggagtccgcgttcgccaacgagggcttcctcggctaccagtacgagtgccccggcctgatcgag |
| | gtccccaccgagcaggaccccagcaagtcctactgggtgatgttcatctccatcaaccccggcgccccggccggcggctccttcaacc |
| | agtacttcgtcggcagcttcaacggcacccacttcgaggccttcgacaaccagtcccgcgtggtggacttcggcaaggactactacgcc |
| | ctgcagaccttcttcaacaccgacccgacctacgggagcgccctgggcatcgcgtgggcctccaactgggagtactccgccttcgtgcc |
| | caccaacccctggcgctcctccatgtccctcgtgcgcaagttctccctcaacaccgagtaccaggccaacccggagacggagctgatc |
| | aacctgaaggccgagccgatcctgaacatcagcaacgccggcccctggagccggttcgccaccaacaccacgttgacgaaggcca |
| | acagctacaacgtcgacctgtccaacagcaccggcaccctggagttcgagctggtgtacgccgtcaacaccacccagacgatctcca |
| | agtccgtgttcgcggacctctccctctggttcaagggcctggaggaccccgaggagtacctccgcatgggcttcgaggtgtccgcgtcct |
| | ccttcttcctggaccgcgggaacagcaaggtgaagttcgtgaaggagaacccctacttcaccaaccgcatgagcgtgaacaaccagc |
| | ccttcaagagcgagaacgacctgtcctactacaaggtgtacggcttgctggaccagaacatcctggagctgtacttcaacgacggcga |
| | cgtcgtgtccaccaacacctacttcatgaccaccgggaacgccctgggctccgtgaacatgacgacgggggtggacaacctgttctac |
| | atcgacaagttccaggtgcgcgaggtcaagtgactTAAGACGCCCGCGCGGCGCACCTGACCTGTTCTCT |
| | CGAGGGCGCCTGTTCTGCCTTGCGAAACAAGCCCCTGGAGCATGCGTGCATGATCGT |
| | CTCTGGCGCCCCGCCGCGCGGTTTGTCGCCCTCGCGGGCGCCGCGGCCGCGGGGGCG |
| | CATTGAAATTGTTGCAAACCCCACCTGACAGATTGAGGGCCCAGGCAGGAAGGCGT |
| | TGAGATGGAGGTACAGGAGTCAAGTAACTGAAAGTTTTTATGATAACTAACAACAA |
| | AGGGTCGTTTCTGGCCAGCGAATGACAAGAACAAGATTCCACATTTCCGTGTAGAGG |
| | CTTGCCATCGAATGTGAGCGGGCGGGCCGCGGACCCGACAAAACCCTTACGACGTG |
| | GTAAGAAAAACGTGGCGGGCACTGTCCCTGTAGCCTGAAGACCAGCAGGAGACGAT |
| | CGGAAGCATCACAGCACAACCGGTgcagcagcagctcggatagtatcgacacactctggacgctggtcgtgtgat |
| | ggactgttgccgccacacttgctgccttgacctgtgaatatccctgccgcttttatcaaacagcctcagtgtgtttgatcttgtgtgtacgcgct |
| | tttgcgagttgctagctgcttgtgctatttgcgaataccacccccagcatccccttccctcgtttcatatcgcttgcatcccaaccgcaacttat |
| | ctacgctgtcctgctatccctcagcgctgctcctgctcctgctcactgcccctcgcacagccttggtttgggctccgcctgtattctcctggtac |
| | 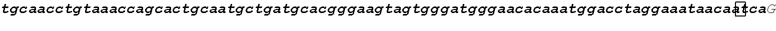 |
| | 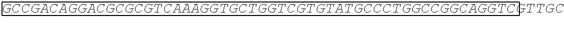 |
| | 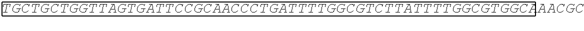 |
| | 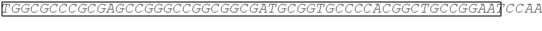 |
| | 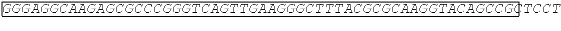 |
| | 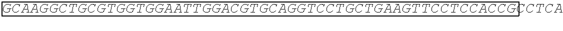 |
| | 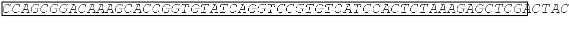 |
| | 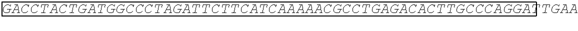 |
| | 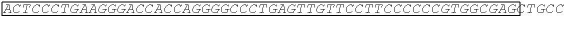 |
| | 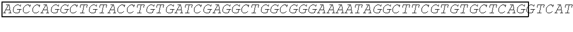 |
| | 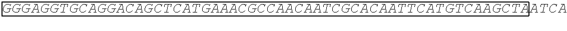 |
| | 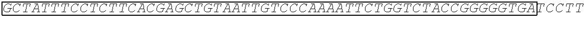 |
| | 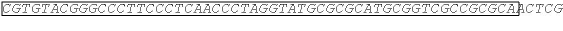 |
| | 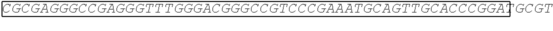 |
| | 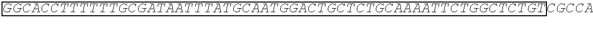 |
| | 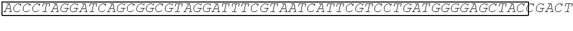 |
| | 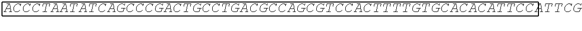 |
| | 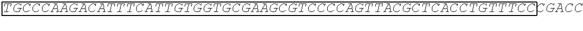 |
| | 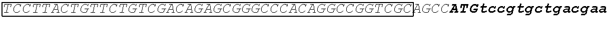 |
| | gtggctgggcctgccctccttcctgttctccgtgttcgtgttctactggtccctgcccatcttcgccatcctgtaccgcatccgcttcgcctccct |
| | gggcaagcgcaacgacatgctggactgggcccgcgccctggtggcctacttccgcgtgaccctgctccaggcgggcgagcacaccct |
| | gtacaagggcggcccctgcctgtacctgtgcaaccaccgctcctgggccgacttcttcatcgacgcctacctgaccgagggccgcgcc |
| | gccctgatgtcccgctggctggtgtacttcgtgttccccgtgttctgcacctcctgcatgatcctgaagggcatcgtgctgttcaagcgcggc |
| | accatcgccgacaaggaggccttcaacgcctggctggaccagaccctgggctcctcccacgtgcccggcctgctggtgtaccccgag |
| | ggccaccgctccaccaagcccgcctccctgcccctgaagcgcggcatgctgcactacgcccactcccgcaagctgcccgtgcagatc |
| | gtggtgacccgcggcaaggacgaggtgctgtccgagaagtcccagtccgtgcacttcggccgcacctgcgtgaccaccttctccaagg |
| | tgctgaagtccgccgactaccccaacttcgaggccttcttcaccgacctccaggccacctgggactcctgctgggccgccacctacggc |
| | ctggaggacctgaagaacgtgccccgcttctccatgcccggcccccaggcctactcctactcctcctccatgtgggtgcagcaactggc |
| | gatcaccctggtgtccatcctggtgttcgcgggcgtgtgctacggctcctggcgcggcctggccgccgccctggccgccacgggcgccg |
| | cccagcaggtggtggccctggtgctggccgcctgggtgggctcctccgtgctgcgctccttcctgTAGttaattaagcagcagcagctc |
| | ggatagtatcgacacactctggacgctggtcgtgtgatggactgttgccgccacacttgctgccttgacctgtgaatatccctgccgcttttatc |
| | aaacagcctcagtgtgtttgatcttgtgtgtacgcgcttttgcgagttgctagctgcttgtgctatttgcgaataccacccccagcatccccttccc |
| | tcgtttcatatcgcttgcatcccaaccgcaacttatctacgctgtcctgctatccctcagcgctgctcctgctcctgctcactgcccctcgcacag |
| | ccttggtttgggctccgcctgtattctcctggtactgcaacctgtaaaccagcactgcaatgctgatgcacgggaagtagtgggatgggaaca |
| | caaatggaagcgtctgcgtgttgggagctggagtcgtgggcttgacgacggcgctgcagctcttgcaggatgtgcctggcgtgcgcg |
| | ttcacgtcgtggctgagaaatatggcgacgaaacgttgacggctggggccgggggctgtggatgccatacgcattgggtacgcgg |
| | ccattggatgggattggtaggcttatggagggataatagagtttttgtcggatccaacgcatgttgatgcagtagcccggtgtgctga |
| | aagtatggaaggatggtgcattggctattcacatgcgctgcccaccccttttcgcaggaaatgtgccggcatcgttggtgcaccgatg |
| | gggaaaatcgacgttcgaccactacatgaaaatgtatacctctgaagatgcagcaactgcgggtgcgaaacggataacggtttggt |
| | aggtgtatgtcgcagcatgtgctggattttgcgggcaactccccctgccacggcccattgcaggtgtcatgttgactggaggatacga |
| | actttcggccgtcaaattcccagaggaggaccccctctgggccgacattgtgcccactgctcttccgcttgctgtttcctgtgtgaaatt |
| | gt |

The construct can be written as 5′DAO1b::CrTUB2:ScSUC2:PmPGH:CvNR:PmAMT03: CrLPAAT2:CvNR::3′DAO1b. Proceeding in the 5′ to 3′ direction, the bold, lowercase sequences represent genomic DNA from CHK22 that permit targeted integration at the DAO1b locus via homologous recombination. The C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics, while the coding region is indicated in lowercase italics. The P. moriformis PmPGH 3′-UTR is indicated by uppercase underlined text followed by a linker, indicated by lowercase, bold italics. The P. moriformis PmAMT03 promoter, indicated by boxed, italicized text, drives the expression of the CrLPAAT2. The initiator ATG and terminator TGA codons of the CrLPAAT2 are indicated by uppercase, bold italics, while the remainder of the gene is indicated by bold italics. The C. vulgaris nitrate reductase 3′-UTR is indicated by lowercase underlined text followed by the CHK22 DAO1b genomic region indicated by bold, lowercase text.

### Strain Growth and Transformation

Unless indicated otherwise, strains described herein were grown in 50-mL conical tube formats with shaking at 200 rpm at 28° C. for 96 hours for lipid production. Culture supernatants were harvested via centrifugation and washed once with MilliQ water. The resulting cellular pellet was lyophilized to dryness and subjected to direct transesterification to generate fatty acid methyl esters (FAMEs) for subsequent quantitation and characterization by gas chromatography-flame ionization detection (GC/FID).

Strains utilized for transformation were grown in vegetative growth medium for 24 hours prior to transformation. Cells were pelleted by centrifugation, resuspended in culture medium, and then re-centrifuged. The resulting cell pellet was re-suspended in culture medium and 5×10⁷ cells plated to the appropriate selection medium and allowed to dry in a sterile biosafety cabinet. Transformants of CHK22 were generated via particle bombardment of previously plated cells using gold nanoparticles. Primary transformants were selected for ability to grow on plates containing sucrose as the sole carbon source. Transformants were grown in lipid production medium and their resulting biomass processed as previously described for fatty acid analysis.

### LCMS Analytical Method

TAG profile analysis was conducted using LCMS. TAGs were extracted from cells into a solution of 3:1 toluene/2-propanol (v/v), by means of mechanical disruption using ceramic beads and vigorous agitation. The filtered extracts were then injected on an Agilent 1290 Infinity II UHPLC system coupled to a 6470B triple quadrupole mass spectrometer and APCI ionization source.

### LCMS Determination of Regioisomers

Qualitative assessment of the regiospecificity of a predominant TAG species of the resulting oil was performed based on the abundance of DAG ions resulting from fragmentation of the TAG species (FIG. 1).

### Example 2: Determination of the Sn-2 Fatty Acid Profile Following Digestion with Pancreatic Porcine Lipase

The fatty acid composition at the sn-2 position was determined after incubating the TAGs with porcine pancreatic lipase. The lipase reaction results in deacylation of the TAG at the sn-1 and sn-3 positions, thereby leaving sn-2-MAGs. The sn-2-MAGs were then isolated using Agilent Bond Elut NH2 propyl solid phase extraction (SPE) cartridges and subjected to fatty acid composition analysis by gas chromatography using a direct transesterification method.

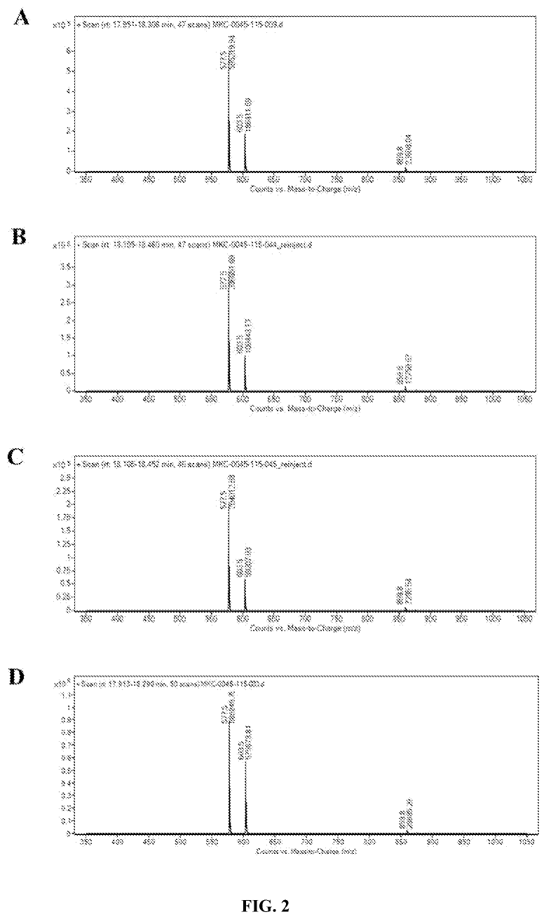

Mass spectra and DAG ion ratios for the non-transgenic strain CHK22 and CHK22 transformed with pCHK385 (D552-1, D552-3, and D552-4) are shown in FIG. 2 and Table 2. FIG. 2 shows mass spectrum of OOP/OPO triglyceride. While regioisomers are not chromatographically separated by this analytical method, the higher ratio of m/z 577 (OP) to m/z 603 (OO) suggests a greater abundance of the OPO regioisomer compared to that of CHK22.

TABLE 2
Screen of primary transformants of CHK22 with pCHK385.

| | FAME Profiles | | | | | | | | | TAG Profile |
|---|---|---|---|---|---|---|---|---|---|---|
| | | | | | | | C18:3 | | | m/z 577/603 |
| Sample | C12:0 | C14:0 | C16:0 | C18:0 | C18:1 | C18:2 | alpha | C20:0 | C20:1 | (OP:OO) |
| pCHK385-1 | 0.04 | 1.20 | 22.15 | 2.86 | 59.82 | 11.81 | 0.57 | 0.19 | 0.13 | 3.19 |
| pCHK385-2 | 0.03 | 1.12 | 21.48 | 3.27 | 61.96 | 9.91 | 0.58 | 0.28 | 0.13 | 1.57 |
| pCHK385-3 | 0.04 | 1.37 | 27.09 | 2.53 | 55.51 | 11.16 | 0.62 | 0.17 | 0.10 | 3.35 |
| pCHK385-4 | 0.04 | 1.24 | 22.05 | 2.78 | 59.11 | 12.66 | 0.59 | 0.18 | 0.14 | 3.45 |
| CHK22 | 0.03 | 1.21 | 26.75 | 3.40 | 59.41 | 7.11 | 0.52 | 0.28 | 0.10 | 1.54 |
| Control | | | | | | | | | | |

Table 2 shows the fatty acid/TAG profiles of four primary transformants of CHK22 transformed with pCHK385. P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization GC/FID. TAG analysis was conducted using the LCMS method as described above.

The fatty acid composition at the sn-2 position of the transgenic lines CHK22, D552; 1, 3, and 4 were further determined after incubating the TAGs with porcine pancreatic lipase. The results were shown in Table 3.

TABLE 3
Determination of the percentage of palmitate at sn-2 position
following digestion with pancreatic porcine lipase, for
the primary transformants of CHK22 with pCHK385.

| | | C16:0 | C18:0 | C18:1 | C18:2 |
|---|---|---|---|---|---|
| CHK22 expressing | Purified Tag Prep 1 | 22.69 | 3.07 | 59.63 | 11.02 |
| CrLPAAT2 | Purified Tag Prep 2 | 22.73 | 3.11 | 59.59 | 10.87 |
| | sn2 Mag Eluate Prep 1 | 42.33 | 0.68 | 46.52 | 6.46 |
| | sn2 Mag Eluate Prep 2 | 41.83 | 0.60 | 46.84 | 6.64 |
| | Purified Tag Prep 1 | 28.42 | 2.40 | 54.63 | 10.63 |
| | Purified Tag Prep 2 | 28.63 | 2.40 | 54.71 | 10.29 |
| | sn2 Mag Eluate Prep 1 | 53.11 | 0.45 | 36.61 | 5.93 |
| | sn2 Mag Eluate Prep 2 | 53.42 | 0.43 | 36.76 | 5.64 |
| | Purified Tag Prep 1 | 22.52 | 25.67 | 59.02 | 12.19 |
| | Purified Tag Prep 2 | 22.84 | 2.72 | 59.05 | 11.74 |
| | sn2 Mag Eluate Prep 1 | 43.68 | 0.63 | 44.58 | 6.52 |
| | sn2 Mag Eluate Prep 2 | 43.72 | 0.67 | 43.89 | 6.51 |
| CHK22 | Purified Tag Prep 1 | 27.68 | 3.43 | 60.22 | 5.24 |
| | Purified Tag Prep 2 | 26.66 | 3.33 | 60.16 | 6.51 |
| | sn2 Mag Eluate Prep 1 | 9.50 | 1.48 | 75.65 | 6.79 |
| | sn2 Mag Eluate Prep 2 | 4.15 | 0.69 | 82.94 | 8.29 |

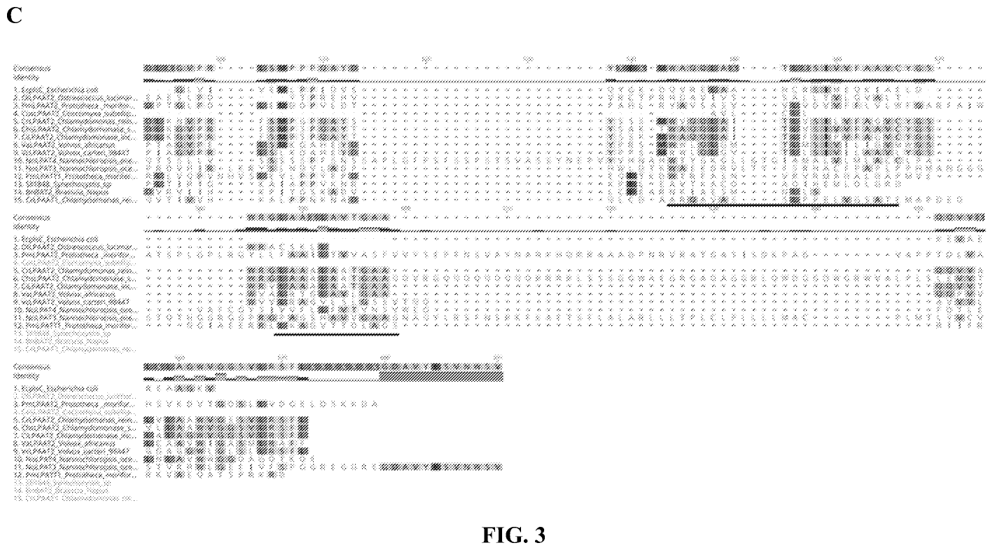

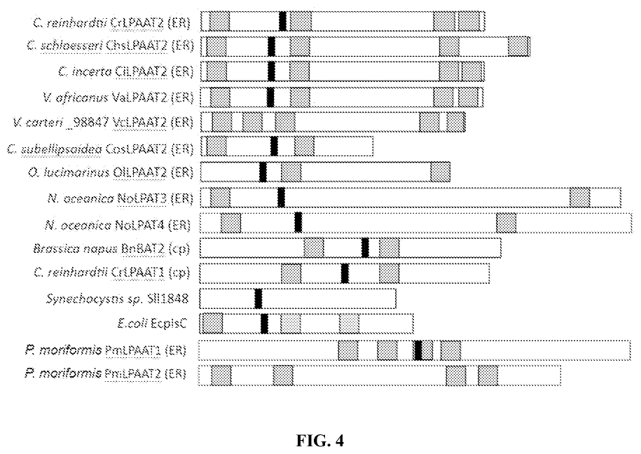

In Table 3, P. moriformis base strain CHK22 is shown as a non-transgenic control. Strains were grown for 120 hours, 200-mL culture in 1-L flask at 200 rpm at 28° C. in lipid production medium. Biomass was harvested, lyophilized to dryness, and subjected to direct transesterification to generate FAMEs for subsequent quantitation and characterization by GC/FID. The fatty acid composition at the sn-2 position was determined with the porcine pancreatic lipase assay as described above. A corresponding bar graph showing the percentage of palmitic acid at the sn-2 position for various strains that had been transformed with pCHK385 is shown in FIG. 5. A graph showing LC-MS results for oil from CHK22 and strain D552-3 is shown in FIG. 6, showing that D552-3 has a similar molecular signature to CHK22, but that the amounts of palmitic acid in the s-2 position had significantly increased.

### Example 3: Expressing C. reinhardtii LPAAT1 (CrLPAAT1) in P. moriformis Strain CHK22

C. reinhardtii CrLPAAT1 was introduced into a P. moriformis strain CHK22. The expression construct pCHK391 contained 5Y and 3Y homology arms to permit targeted integration of CrLPAAT1 into the genome and is shown in Table 4.

TABLE 4
Intcgrative sequences for the transformation of P. moriformis with pCHK391
encoding CrLPAAT1.

| SEQ ID | |
|---|---|
| NO: | Sequence |
| 2 | gctcttccgcttgcccgcaccctcgttgatctgggagccctgcgcagccccttaaatcatctcagtcaggtttctatgttcaactgag |
| | cctaaagggctttcgtcacgcgcacgagcacacgtatatcggctacgcagtctctcagaagcggtagaacagttcgcaagccct |
| | cgtcggtcgaaaacttgcgccagtactattgaattaaattaaatgatcgaatgagacgcgaaacttttgcagaatgccactgagt |
| | ttgcccagagaatgggagtggcgccattcaccatccgcctgtgcacggcctgattcgccgagacgatgaatggcgagaccagg |
| | gagcggcttgcgagccccgagccggtagcaggaataacggtcgacaatcttcctgtccaattactggcaaccattagaaagagc |
| | cggagcgcgttgaaattctgcaatcgagtaatttttcgatgcggcgggcctgctgaaccctaaggctccggcctatgtttaaggcg |
| | atccaagatgcacgcggccccaggcacgtgtctcaagcacaaaccccagccttagtttcgagactttgggagatagcggcagat |
| | atctagtttggcattttgtatattaattaagtctcgcaatggagcgctctgatgcggtgcagcgtcggctgcagcacctggcagtgg |
| | 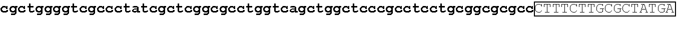 |
| | 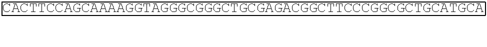 |
| | 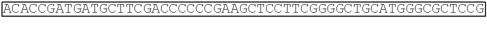 |
| | 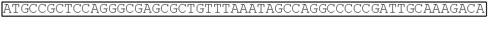 |
| | 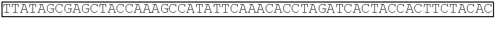 |
| | 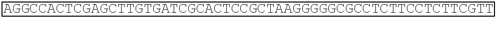 |
| | 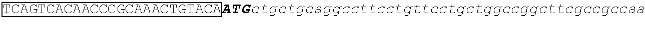 |
| | gatcagcgcctccatgacgaacgagacgtccgaccgccccctggtgcacttcacccccaacaagggctggatgaacgaccccaa |
| | cggcctgtggtacgacgagaaggacgccaagtggcacctgtacttccagtacaacccgaacgacaccgtctgggggacgcccttg |
| | ttctggggccacgccacgtccgacgacctgaccaactgggaggaccagcccatcgccatcgccccgaagcgcaacgactccggc |
| | gccttctccggctccatggtggtggactacaacaacacctccggcttcttcaacgacaccatcgacccgcgccagcgctgcgtggcc |
| | atctggacctacaacaccccggagtccgaggagcagtacatctcctacagcctggacggcggctacaccttcaccgagtaccaga |
| | agaaccccgtgctggccgccaactccacccagttccgcgacccgaaggtcttctggtacgagccctcccagaagtggatcatgacc |
| | gcggccaagtcccaggactacaagatcgagatctactcctccgacgacctgaagtcctggaagctggagtccgcgttcgccaacg |
| | agggcttcctcggctaccagtacgagtgccccggcctgatcgaggtccccaccgagcaggaccccagcaagtcctactgggtgatg |
| | ttcatctccatcaaccccggcgccccggccggcggctccttcaaccagtacttcgtcggcagcttcaacggcacccacttcgaggcct |
| | tcgacaaccagtcccgcgtggtggacttcggcaaggactactacgccctgcagaccttcttcaacaccgacccgacctacgggagc |
| | gccctgggcatcgcgtgggcctccaactgggagtactccgccttcgtgcccaccaacccctggcgctcctccatgtccctcgtgcgca |
| | agttctccctcaacaccgagtaccaggccaacccggagacggagctgatcaacctgaaggccgagccgatcctgaacatcagca |
| | acgccggcccctggagccggttcgccaccaacaccacgttgacgaaggccaacagctacaacgtcgacctgtccaacagcaccg |
| | gcaccctggagttcgagctggtgtacgccgtcaacaccacccagacgatctccaagtccgtgttcgcggacctctccctctggttcaa |
| | gggcctggaggaccccgaggagtacctccgcatgggcttcgaggtgtccgcgtcctccttcttcctggaccgcgggaacagcaagg |
| | tgaagttcgtgaaggagaacccctacttcaccaaccgcatgagcgtgaacaaccagcccttcaagagcgagaacgacctgtccta |
| | ctacaaggtgtacggcttgctggaccagaacatcctggagctgtacttcaacgacggcgacgtcgtgtccaccaacacctacttcat |
| | gaccaccgggaacgccctgggctccgtgaacatgacgacgggggtggacaacctgttctacatcgacaagttccaggtgcgcgag |
| | gtcaagtgactTAAGACGCCCGCGCGGCGCACCTGACCTGTTCTCTCGAGGGCGCCTGTT |
| | CTGCCTTGCGAAACAAGCCCCTGGAGCATGCGTGCATGATCGTCTCTGGCGCCCCG |
| | CCGCGCGGTTTGTCGCCCTCGCGGGCGCCGCGGCCGCGGGGGCGCATTGAAATTG |
| | TTGCAAACCCCACCTGACAGATTGAGGGCCCAGGCAGGAAGGCGTTGAGATGGAG |
| | GTACAGGAGTCAAGTAACTGAAAGTTTTTATGATAACTAACAACAAAGGGTCGTT |
| | TCTGGCCAGCGAATGACAAGAACAAGATTCCACATTTCCGTGTAGAGGCTTGCCAT |
| | CGAATGTGAGCGGGGGGCCGCGGACCCGACAAAACCCTTACGACGTGGTAAGAA |
| | AAACGTGGCGGGCACTGTCCCTGTAGCCTGAAGACCAGCAGGAGACGATCGGAAG |
| | CATCACAGCACAACCGGTgcagcagcagctcggatagtatcgacacactctggacgctgtcgtgtgatggactgtt |
| | gccgccacacttgctgccttgacctgtgaatatccctgccgcttttatcaaacagcctcagtgtgtttgatcttgtgtgtacgcgcttttgcg |
| | agttgctagctgcttgtgctatttgcgaataccacccccagcatccccttccctcgtttcatatcgcttgcatcccaaccgcaacttatcta |
| | cgctgtcctgctatccctcagcgctgctcctgctcctgctcactgcccctcgcacagccttggtttgggctccgcctgtattctcctggtact |
| | gcaacctgtaaaccagcactgcaatgctgatgcacgggaagtagtgggatgggaacacaaatggacctaggaaataacaatca |
| | 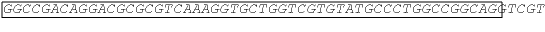 |
| | 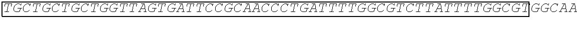 |
| | 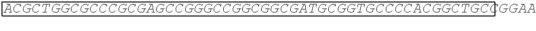 |
| | 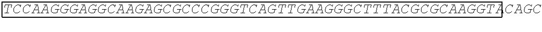 |
| | 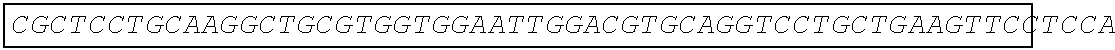 |
| | 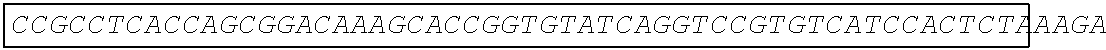 |
| | 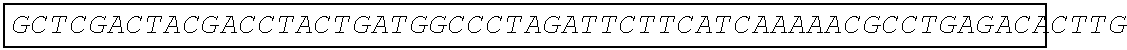 |
| | 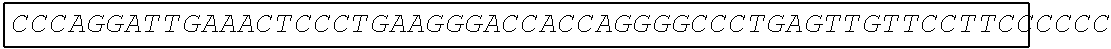 |
| | 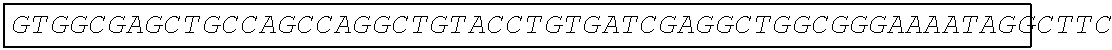 |
| | 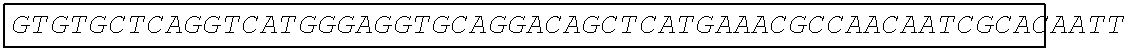 |
| | 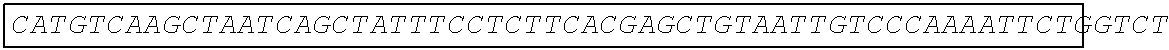 |
| | 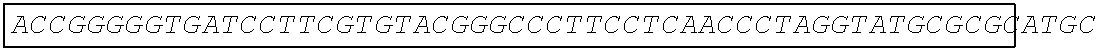 |
| | 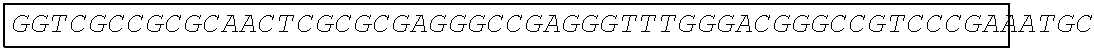 |
| | 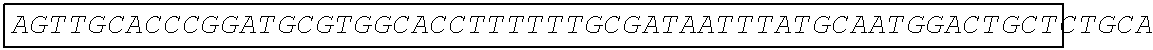 |
| | 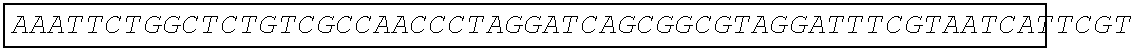 |
| | 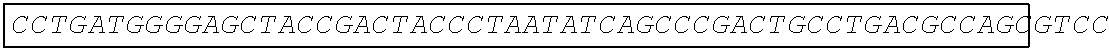 |
| | 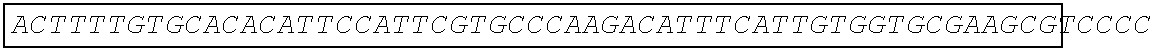 |
| | 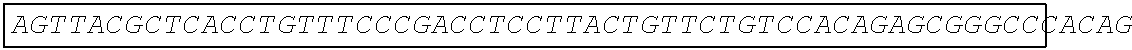 |
| | 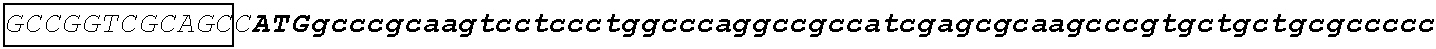 |
| | agctcaacgtgccccgcatgtccggcatcaccgccctgcccatggagcgccgccccctgcccgtggccccctccccctccgccaag |
| | cccgagctgcccgcccgctccgccctggtgtgccacgccgccgccgcctccgtgcccctgcccaactccgactccgccccccagcc |
| | caacgtgctgctggccaagatccgcgccatcatgttcttcgcctggtccttcctgctgtccctgcccctgttcgtgaccatgatggtgatg |
| | gcccccctggtgctggccttcgacaagtaccgccgcctggcccagcacttcgtgaacaacctgtgggcctgcgcctccaccgccccc |
| | ttctacaaggtgaccatcatcggccgcgagaacctgcccccccccgacaagcccgtggtgtacgtggccaaccaccagtccttcctg |
| | gacatctactccctgttccacctccagcgccccttcaagttcatctccaagacctccaacttcctgatccccatcatcggctggtccatgt |
| | tcctgacgggccacgtgatgatcaaccgcgtggaccgccgctcccagctcaagtgcctccagcagtgccgcgacctgctggccga |
| | gggcgcccccgtgctgttcttccccgagggcacccgctccctggactgcaagatggcgggcttcaagaagggcgccttctccgtggc |
| | cgccaaggcgggcgtggaggtggtgcccatcaccctgctgggcacgggctccctgatgccctccggcaaggagtcccagctccgc |
| | cccggccaggtgaccatcgtggtgcacaaggccctgccccccaacaagaacgccgaccagctctgcgacgccgcccgccaggc |
| | cgtggcctcctccctgccccccgagctggtgggctccgcgaccgagatggcccccgacgagcagTAGttaattaagcagcagca |
| | gctcggatagtatcgacacactctggacgctggtcgtgtgatggactgttgccgccacacttgctgccttgacctgtgaatatccctgccg |
| | cttttatcaaacagcctcagtgtgtttgatcttgtgtgtacgcgcttttgcgagttgctagctgcttgtgctatttgcgaataccacccccagcat |
| | ccccttccctcgtttcatatcgcttgcatcccaaccgcaacttatctacgctgtcctgctatccctcagcgctgctcctgctcctgctcactgc |
| | ccctcgcacagccttggtttgggctccgcctgtattctcctggtactgcaacctgtaaaccagcactgcaatgctgatgcacgggaagtag |
| | tgggatgggaacacaaatggaagcgtctgcgtgttgggagctggagtcgtgggcttgacgacggcgctgcagctcttgcaggatg |
| | tgcctggcgtgcgcgttcacgtcgtggctgagaaatatggcgacgaaacgttgacggctggggccggcgggctgtggatgccat |
| | acgcattgggtacgcggccattggatgggattggtaggcttatggagggataatagagtttttgtcggatccaacgcatgttgatg |
| | cagtagcccggtgtgctgaaagtatggaaggatggtgcattggctattcacatgcgctgcccaccccttttcgcaggaaatgtgc |
| | cggcatcgttggtgcaccgatggggaaaatcgacgttcgaccactacatgaaaatgtatacctctgaagatgcagcaactgcgg |
| | gtgcgaaacggataacggtttggtaggtgtatgtcgcagcatgtgctggattttgcgggcaactccccctgccacggcccattgc |
| | aggtgtcatgttgactggaggatacgaactttcggccgtcaaattcccagaggaggaccccctctgggccgacattgtgcccact |
| | gctcttccgcttgctgtttcctgtgtgaaattgt |

The construct can be written as 5′DAO1b::CrTUB2:ScSUC2:PmPGH:CvNR:PmAMT03:CrLPAAT1:CvNR::3′DAO1b. Proceeding in the 5′ to 3′ direction, bold, lowercase sequences represent genomic DNA from CHK22 that permit targeted integration at the DAO1b locus via homologous recombination, the C. reinhardtii β-tubulin promoter driving expression of the yeast sucrose invertase gene (conferring the ability of CHK22 to metabolize sucrose) is indicated by boxed, uppercase text. The initiator ATG and terminator TAA for invertase are indicated by uppercase, bold italics while the coding region is indicated in lowercase italics. The P. moriformis PmPGH 3′-UTR is indicated by uppercase underlined text followed by a linker, indicated by lowercase, bold italics. The P. moriformis PmAMT03 promoter, indicated by boxed italicized text, drives the expression of the CrLPAAT1. The initiator ATG and terminator TGA codons of the CrLPAAT1 are indicated by uppercase, bold italics, while the remainder of the gene is indicated by bold italics. The C. vulgaris nitrate reductase 3′-UTR is indicated by lowercase, underlined text followed by the CHK22 DAO1b genomic region indicated by bold, lowercase text.

Table 5 shows the fatty acid/TAG profiles of four primary transformants of CHK22 transformed with pCHK391. The P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 5
Screen of primary transformants of CHK22 with pCHK391.

| | FAME Profiles | | | | | | | | | TAG Profile |
|---|---|---|---|---|---|---|---|---|---|---|
| | | | | | | | C18:3 | | | m/z 577/603 |
| Sample | C12:0 | C14:0 | C16:0 | C18:0 | C18:1 | C18:2 | alpha | C20:0 | C20:1 | (OP:OO) |
| pCHK391-1 | 0.04 | 0.72 | 20.49 | 2.38 | 59.35 | 14.38 | 0.78 | 0.27 | 0.26 | 1.52 |
| pCHK391-2 | 0.04 | 0.71 | 20.25 | 2.30 | 59.33 | 14.75 | 0.73 | 0.26 | 0.26 | 1.52 |
| pCHK391-3 | 0.04 | 0.71 | 20.05 | 2.27 | 59.36 | 14.90 | 0.77 | 0.26 | 0.26 | 1.48 |
| pCHK391-4 | 0.03 | 1.08 | 24.60 | 2.93 | 59.71 | 9.39 | 0.55 | 0.29 | 0.15 | 1.59 |
| CHK22 | 0.03 | 1.21 | 26.75 | 3.40 | 59.41 | 7.11 | 0.52 | 0.28 | 0.10 | 1.54 |
| Control | | | | | | | | | | |

### Example 4: Expressing Brassica napus BnBAT2 in P. moriformis Strain CHK22

B. napus BnBAT2 was introduced into a P. moriformis strain CHK22. The expression construct pCHK384 contained 5Y and 3Y homology arms to permit targeted integration of BnBAT2 into the genome and is shown in Table 6.

TABLE 6
Integrative sequences for the transformation of P. moriformis with pCHK384
encoding BnBAT2.

| SEQ ID | |
|---|---|
| NO: | Sequence |
| 3 | gctcttccgcttgcccgcaccctcgttgatctgggagccctgcgcagccccttaaatcatctcagtcaggtttctatgttcaactgagcc |
| | taaagggctttcgtcacgcgcacgagcacacgtatatcggctacgcagtctctcagaagcggtagaacagttcgcaagccctcgtc |
| | ggtcgaaaacttgcgccagtactattgaattaaattaaatgatcgaatgagacgcgaaacttttgcagaatgccactgagtttgccc |
| | agagaatgggagtggcgccattcaccatccgcctgtgcacggcctgattcgccgagacgatgaatggcgagaccagggagcggct |
| | tgcgagccccgagccggtagcaggaataacggtcgacaatcttcctgtccaattactggcaaccattagaaagagccggagcgcg |
| | ttgaaattctgcaatcgagtaatttttcgatgcggcgggcctgctgaaccctaaggctccggcctatgtttaaggcgatccaagatgc |
| | acgcggccccaggcacgtgtctcaagcacaaaccccagccttagtttcgagactttgggagatagcggcagatatctagtttggcat |
| | tttgtatattaattaagtctcgcaatggagcgctctgatgcggtgcagcgtcggctgcagcacctggcagtggcgctggggtcgccct |
| | 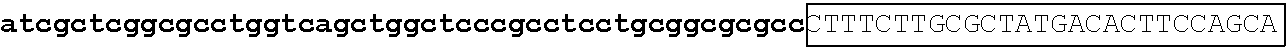 |
| | 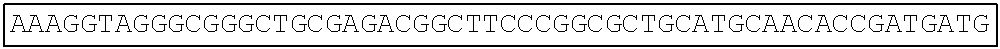 |
| | 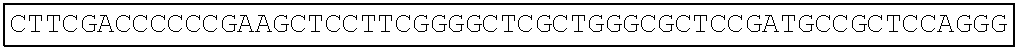 |
| | 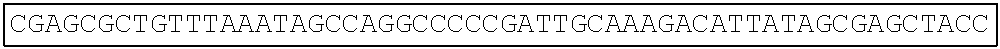 |
| | 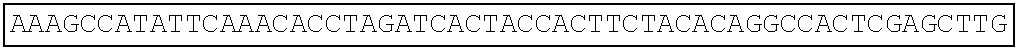 |
| | 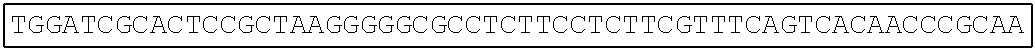 |
| | 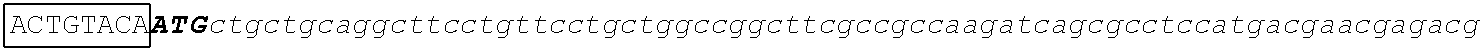 |
| | tccgaccgccccctggtgcacttcacccccaacaagggctggatgaacgaccccaacggcctgtggtacgacgagaaggacgccaa |
| | gtggcacctgtacttccagtacaacccgaacgacaccgtctgggggacgcccttgttctggggccacgccacgtccgacgacctgacc |
| | aactgggaggaccagcccatcgccatcgccccgaagcgcaacgactccggcgccttctccggctccatggtggtggactacaacaac |
| | acctccggcttcttcaacgacaccatcgacccgcgccagcgctgcgtggccatctggacctacaacaccccggagtccgaggagcag |
| | tacatctcctacagcctggacggcggctacaccttcaccgagtaccagaagaaccccgtgctggccgccaactccacccagttccgcg |
| | acccgaaggtcttctggtacgagccctcccagaagtggatcatgaccgcggccaagtcccaggactacaagatcgagatctactcctc |
| | cgacgacctgaagtcctggaagctggagtccgcgttcgccaacgagggcttcctcggctaccagtacgagtgccccggcctgatcgag |
| | gtccccaccgagcaggaccccagcaagtcctactgggtgatgttcatctccatcaaccccggcgccccggccggcggctccttcaacc |
| | agtacttcgtcggcagcttcaacggcacccacttcgaggccttcgacaaccagtcccgcgtggtggacttcggcaaggactactacgcc |
| | ctgcagaccttcttcaacaccgacccgacctacgggagcgccctgggcatcgcgtgggcctccaactgggagtactccgccttcgtgcc |
| | caccaacccctggcgctcctccatgtccctcgtgcgcaagttctccctcaacaccgagtaccaggccaacccggagacggagctgatc |
| | aacctgaaggccgagccgatcctgaacatcagcaacgccggcccctggagccggttcgccaccaacaccacgttgacgaaggcca |
| | acagctacaacgtcgacctgtccaacagcaccggcaccctggagttcgagctggtgtacgccgtcaacaccacccagacgatctcca |
| | agtccgtgttcgcggacctctccctctggttcaagggcctggaggaccccgaggagtacctccgcatgggcttcgaggtgtccgcgtcct |
| | ccttcttcctggaccgcgggaacagcaaggtgaagttcgtgaaggagaacccctacttcaccaaccgcatgagcgtgaacaaccagc |
| | ccttcaagagcgagaacgacctgtcctactacaaggtgtacggcttgctggaccagaacatcctggagctgtacttcaacgacggcga |
| | cgtcgtgtccaccaacacctacttcatgaccaccgggaacgccctgggctccgtgaacatgacgacgggggtggacaacctgttctac |
| | atcgacaagttccaggtgcgcgaggtcaagtgactTAAGACGCCCGCGCGGCGCACCTGACCTGTTCTCT |
| | CGAGGGCGCCTGTTCTGCCTTGCGAAACAAGCCCCTGGAGCATGCGTGCATGATCGT |
| | CTCTGGCGCCCCGCCGCGCGGTTTGTCGCCCTCGCGGGCGCCGCGGCCGCGGGGGCG |
| | CATTGAAATTGTTGCAAACCCCACCTGACAGATTGAGGGCCCAGGCAGGAAGGCGT |
| | TGAGATGGAGGTACAGGAGTCAAGTAACTGAAAGTTTTTATGATAACTAACAACAA |
| | AGGGTCGTTTCTGGCCAGCGAATGACAAGAACAAGATTCCACATTTCCGTGTAGAGG |
| | CTTGCCATCGAATGTGAGCGGGCGGGCCGCGGACCCGACAAAACCCTTACGACGTG |
| | GTAAGAAAAACGTGGCGGGCACTGTCCCTGTAGCCTGAAGACCAGCAGGAGACGAT |
| | CGGAAGCATCACAGCACAACCGGTgcagcagcagctcggatagtatcgacacactctggacgctggtcgtgtgat |
| | ggactgttgccgccacacttgctgccttgacctgtgaatatccctgccgcttttatcaaacagcctcagtgtgtttgatcttgtgtgtacgcgct |
| | tttgcgagttgctagctgcttgtgctatttgcgaataccacccccagcatccccttccctcgtttcatatcgcttgcatcccaaccgcaacttat |
| | ctacgctgtcctgctatccctcagcgctgctcctgctcctgctcactgcccctcgcacagccttggtttgggctccgcctgtattctcctggtac |
| | 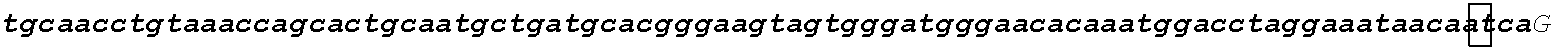 |
| | 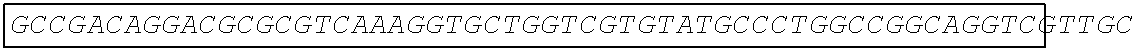 |
| | 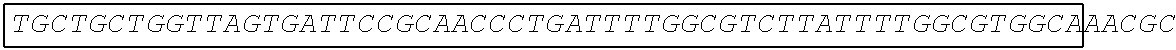 |
| | 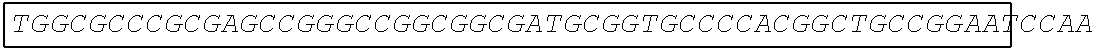 |
| | 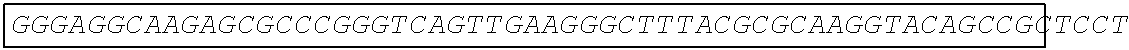 |
| | 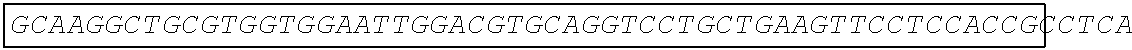 |
| | 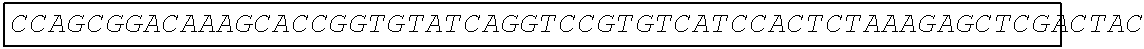 |
| | 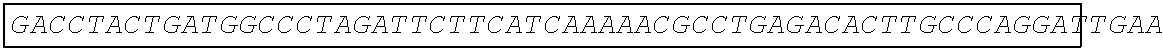 |
| | 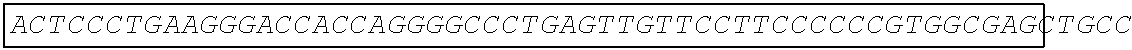 |
| | 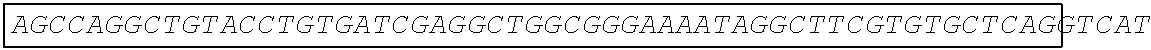 |
| | 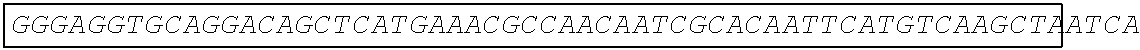 |
| | 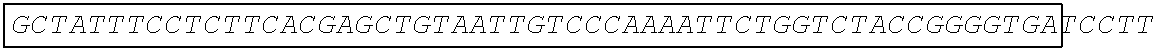 |
| | 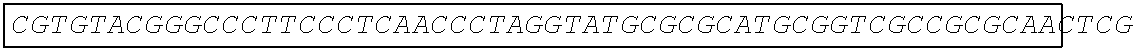 |
| | 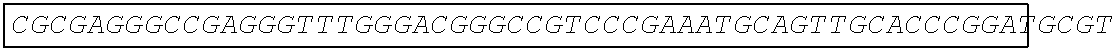 |
| | 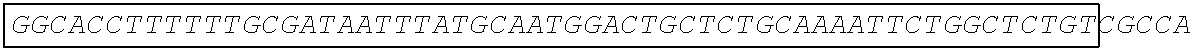 |
| | 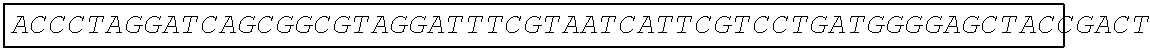 |
| | 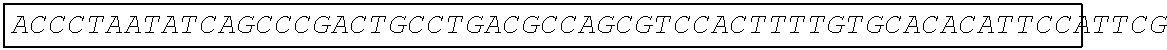 |
| | 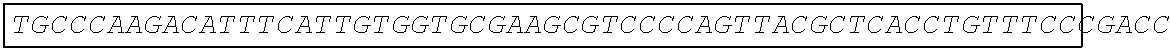 |
| | 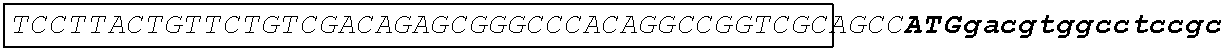 |
| | ccgcggcgtgtcctcccaccccccctactactccaagcccatctgctcctcccagtcctccctgatccgcatccccatctccaagggctgct |
| | gcttcgcccgctcctccaacctgatcacctccctgcacgccgcctcccgcggcgtgacccgccgcacctccggcgtgcagtggtgctac |
| | cgctccatccgcttcgaccccttcaaggtgaacgacaagaactcccgcaccgtgaccgtgcgctccgacctgtccggcgccgccaccc |
| | ccgagtccacctaccccgagcccgagatcaagctgtcctcccgcctgcgcggcatctgcttctgcctggtgggggcatctccgccatcg |
| | tgctgatcgtgctgatgatcatcggccaccccttcgtgctgctgttcgaccgctaccgccgcaagttccaccacttcatcgccaagctgtgg |
| | gcctccatctccatctaccccttctacaagaccgacatccagggcctggagaacctgccctcctccgacaccccctgcgtgtacgtgtcc |
| | aaccaccagtccttcctggacatctacaccctgctgtccctgggccagtcctacaagttcatctccaagacgggcatcttcgtgatccccg |
| | tgatcggctgggccatgtccatgatgggcgtggtgcccctgaagcgcatggacccccgctcccaggtggactgcctgaagcgctgcat |
| | ggagctggtgaagaagggcgcctccgtgttcttcttccccgagggcacccgctccaaggacggccgcctgggccccttcaagaaggg |
| | cgccttcaccatcgccgccaagacgggcgtgcccgtggtgcccatcaccctgatgggcacgggcaagatcatgcccacgggctccga |
| | gggcatcctgaaccacggcgacgtgcgcgtgatcatccacaagcccatctacggctccaaggccgacgtgctgtgcgaggaggccc |
| | gcaacaagatcgccgagtccatgaacctgctgtccTAGttaattaagcagcagcagctcggatagtatcgacacactctggacgctggt |
| | cgtgtgatggactgttgccgccacacttgctgccttgacctgtgaatatccctgccgcttttatcaaacagcctcagtgtgtttgatcttgtgtgta |
| | cgcgcttttgcgagttgctagctgcttgtgctatttgcgaataccacccccagcatccccttccctcgtttcatatcgcttgcatcccaaccgcaa |
| | cttatctacgctgtcctgctatccctcagcgctgctcctgctcctgctcactgcccctcgcacagccttggtttgggctccgcctgtattctcctg |
| | gtactgcaacctgtaaaccagcactgcaatgctgatgcacgggaagtagtgggatgggaacacaaatggaagcgtctgcgtgttgggag |
| | ctggagtcgtgggcttgacgacggcgctgcagctcttgcaggatgtgcctggcgtgcgcgttcacgtcgtggctgagaaatatggcg |
| | acgaaacgttgacggctggggccggcgggctgtggatgccatacgcattgggtacgcggccattggatgggattggtaggcttatg |
| | gagggataatagagtttttgtcggatccaacgcatgttgatgcagtagcccggtgtgctgaaagtatggaaggatggtgcattggct |
| | attcacatgcgctgcccaccccttttcgcaggaaatgtgccggcatcgttggtgcaccgatggggaaaatcgacgttcgaccactac |
| | atgaaaatgtatacctctgaagatgcagcaactgcgggtgcgaaacggataacggtttggtaggtgtatgtcgcagcatgtgctgg |
| | attttgcgggcaactccccctgccacggcccattgcaggtgtcatgttgactggaggatacgaactttcggccgtcaaattcccagag |
| | gaggaccccctctgggccgacattgtgcccactgctcttccgcttgctgtttcctgtgtgaaattgt |

The construct can be written as 5′DAO1b::CrTUB2:ScSUC2:PmPGH:CvNR:PmAMT03:BnBAT2:CvNR::3′DAO1b. Proceeding in the 5′ to 3′ direction, bold, lowercase sequences represent genomic DNA from CHK22 that permit targeted integration at the DAO1b locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text followed by a linker, indicated by lowercase, bold italics. The P. moriformis PmAMT03 promoter, indicated by boxed italicized text, drives the expression of the BnBAT2. The initiator ATG and terminator TGA codons of the BnBAT2 are indicated by uppercase, bold italics, while the remainder of the gene is indicated by bold italics. The C. vulgaris nitrate reductase 3′-UTR is indicated by lowercase underlined text followed by the CHK22 DAO1b genomic region indicated by bold, lowercase text.

Table 7 shows the fatty acid/TAG profiles of four primary transformants of CHK22 transformed with pCHK384. P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 7
Screen of primary transformants of CHK22 with pCHK384.

| | FAME Profiles | | | | | | | | | TAG Profile |
|---|---|---|---|---|---|---|---|---|---|---|
| | | | | | | | C18:3 | | | m/z 577/603 |
| Sample | C12:0 | C14:0 | C16:0 | C18:0 | C18:1 | C18:2 | alpha | C20:0 | C20:1 | (OP:OO) |
| pCHK384-1 | 0.04 | 0.65 | 19.44 | 2.43 | 60.06 | 14.75 | 0.79 | 0.27 | 0.28 | 1.53 |
| pCHK384-2 | 0.03 | 0.97 | 23.19 | 2.91 | 60.48 | 10.10 | 0.58 | 0.28 | 0.18 | 1.57 |
| pCHK384-3 | 0.04 | 0.65 | 19.62 | 2.48 | 59.53 | 15.02 | 0.79 | 0.27 | 0.29 | 1.45 |
| pCHK384-4 | 0.04 | 0.65 | 19.35 | 2.40 | 59.82 | 15.07 | 0.79 | 0.27 | 0.29 | 1.52 |
| CHK22 | 0.03 | 1.21 | 26.75 | 3.40 | 59.41 | 7.11 | 0.52 | 0.28 | 0.10 | 1.54 |
| Control | | | | | | | | | | |

### Example 5: Expressing Nannochloropsis NoLPAT4 in P. moriformis Strain CHK22

Nannochloropsis NoLPAT4 was introduced into a P. moriformis strain CHK22. The expression construct pCHK386 contained 5Y and 3Y homology arms to permit targeted integration of NoLPAT4 into the genome and is shown in Table 8.

TABLE 8
Integrative sequences for the transformation of P. moriformis with pCHK386
encoding NoLPAT4.

| SEQ | |
|---|---|
| ID | |
| NO: | Sequence |
| 4 | gctcttccgcttgcccgcaccctcgttgatctgggagccctgcgcagccccttaaatcatctcagtcaggtttctatgttcaactgagcct |
| | aaagggctttcgtcacgcgcacgagcacacgtatatcggctacgcagtctctcagaagcggtagaacagttcgcaagccctcgtcgg |
| | tcgaaaacttgcgccagtactattgaattaaattaaatgatcgaatgagacgcgaaacttttgcagaatgccactgagtttgcccaga |
| | gaatgggagtggcgccattcaccatccgcctgtgcacggcctgattcgccgagacgatgaatggcgagaccagggagcggcttgcg |
| | agccccgagccggtagcaggaataacggtcgacaatcttcctgtccaattactggcaaccattagaaagagccggagcgcgttgaa |
| | attctgcaatcgagtaatttttcgatgcggcgggcctgctgaaccctaaggctccggcctatgtttaaggcgatccaagatgcacgcgg |
| | ccccaggcacgtgtctcaagcacaaaccccagccttagtttcgagactttgggagatagcggcagatatctagtttggcattttgtatat |
| | taattaagtctcgcaatggagcgctctgatgcggtgcagcgtcggctgcagcacctggcagtggcgctggggtcgccctatcgctcgg |
| | 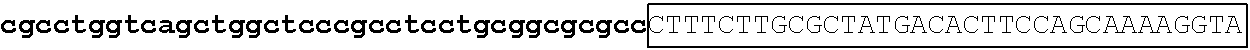 |
| | 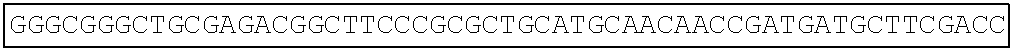 |
| | 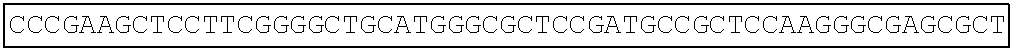 |
| | 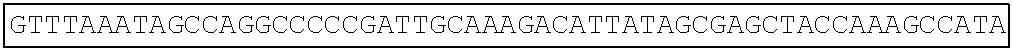 |
| | 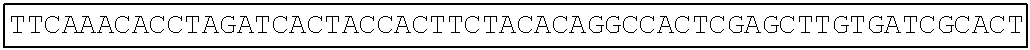 |
| | 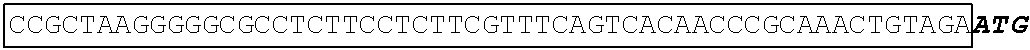 |
| | ctgctgcaggccttcctgttcctgctggccggcttcgccgccaagatcagcgcctccatgacgaacgagacgtccgaccgccccctggtg |
| | cacttcacccccaacaagggctggatgaacgaccccaacggcctgtggtacgacgagaaggacgccaagtggcacctgtacttccagt |
| | acaacccgaacgacaccgtctgggggacgcccttgttctggggccacgccacgtccgacgacctgaccaactgggaggaccagccca |
| | tcgccatcgccccgaagcgcaacgactccggcgccttctccggctccatggtggtggactacaacaacacctccggcttcttcaacgaca |
| | ccatcgacccgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctggtacgagc |
| | cctcccagaagtggatcatgaccgcggccaagtcccaggactacaagatcgagatctactcctccgacgacctgaagtcctggaagctg |
| | gagtccgcgttcgccaacgagggcttcctcggctaccagtacgagtgccccggcctgatcgaggtccccaccgagcaggaccccagca |
| | agtcctactgggtgatgttcatctccatcaaccccggcgccccggccggcggctccttcaaccagtacttcgtcggcagcttcaacggcac |
| | ccacttcgaggccttcgacaaccagtcccgcgtggtggacttcggcaaggactactacgccctgcagaccttcttcaacaccgacccgac |
| | ctacgggagcgccctgggcatcgcgtgggcctccaactgggagtactccgccttcgtgcccaccaacccctggcgctcctccatgtccctc |
| | gtgcgcaagttctccctcaacaccgagtaccaggccaacccggagacggagctgatcaacctgaaggccgagccgatcctgaacatc |
| | agcaacgccggcccctggagccggttcgccaccaacaccacgttgacgaaggccaacagctacaacgtcgacctgtccaacagcacc |
| | ggcaccctggagttcgagctggtgtacgccgtcaacaccacccagacgatctccaagtccgtgttcgcggacctctccctctggttcaagg |
| | gcctggaggaccccgaggagtacctccgcatgggcttcgaggtgtccgcgtcctccttcttcctggaccgcgggaacagcaaggtgaag |
| | ttcgtgaaggagaacccctacttcaccaaccgcatgagcgtgaacaaccagcccttcaagagcgagaacgacctgtcctactacaagg |
| | tgtacggcttgctggaccagaacatcctggagctgtacttcaacgacggcgacgtcgtgtccaccaacacctacttcatgaccaccggga |
| | acgccctgggctccgtgaacatgacgacgggggtggacaacctgttctacatcgacaagttccaggtgcgcgaggtcaagtgactTA |
| | AGACGCCCGCGCGGCGCACCTGACCTGTTCTCTCGAGGGCGCCTGTTCTGCCTTGCGA |
| | AACAAGCCCCTGGAGCATGCGTGCATGATCGTCTCTGGCGCCCCGCCGCGCGGTTTGT |
| | CGCCCTCGCGGGCGCCGCGGCCGCGGGGGCGCATTGAAATTGTTGCAAACCCCACCT |
| | GACAGATTGAGGGCCCAGGCAGGAAGGCGTTGAGATGGAGGTACAGGAGTCAAGTA |
| | ACTGAAAGTTTTTATGATAACTAACAACAAAGGGTCGTTTCTGGCCAGCGAATGACA |
| | AGAACAAGATTCCACATTTCCGTGTAGAGGCTTGCCATCGAATGTGAGCGGGCGGGC |
| | CGCGGACCCGACAAAACCCTTACGACGTGGTAAGAAAAACGTGGCGGGCACTGTCCC |
| | TGTAGCCTGAAGACCAGCAGGAGACGATCGGAAGCATCACAGCACAACCGGTgcagcag |
| | cagctcggatagtatcgacacactctggacgctggtcgtgtgatggactgttgccgccacacttgctgccttgacctgtgaatatccctgcc |
| | gcttttatcaaacagcctcagtgtgtttgatcttgtgtgtacgcgcttttgcgagttgctagctgcttgtgctatttgcgaataccacccccagca |
| | tccccttccctcgtttcatatcgcttgcatcccaaccgcaacttatctacgctgtcctgctatccctcagcgctgctcctgctcctgctcactgcc |
| | cctcgcacagccttggtttgggctccgcctgtattctcctggtactgcaacctgtaaaccagcactgcaatgctgatgcacgggaagtagtg |
| | 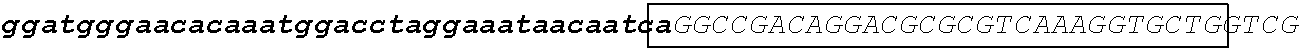 |
| | 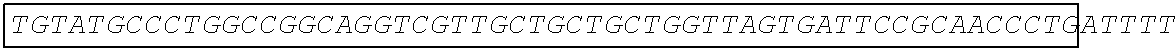 |
| | 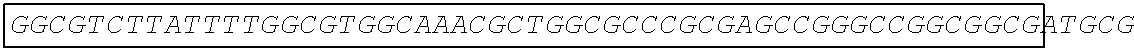 |
| | 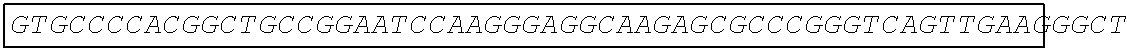 |
| | 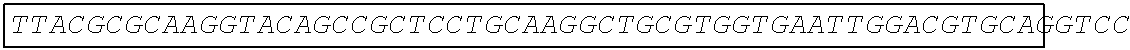 |
| | 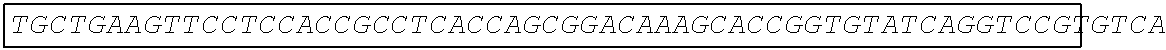 |
| | 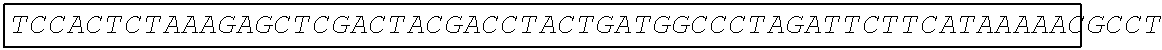 |
| | 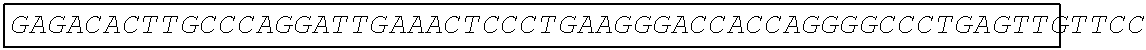 |
| | 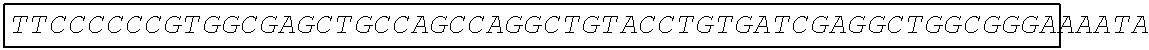 |
| | 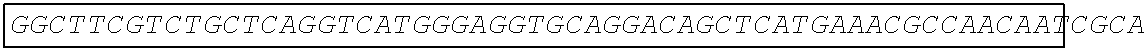 |
| | 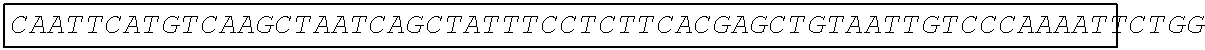 |
| | 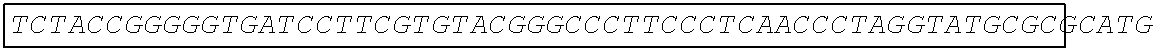 |
| | 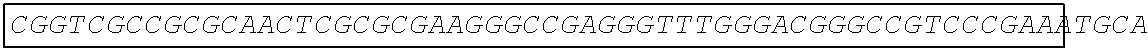 |
| | 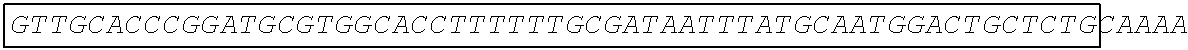 |
| | 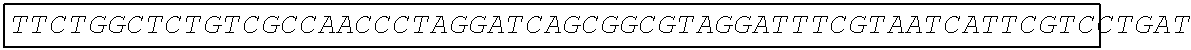 |
| | 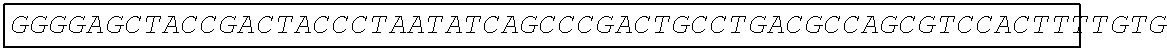 |
| | 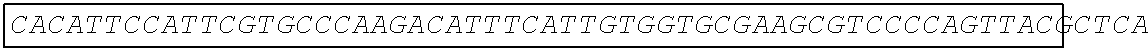 |
| | 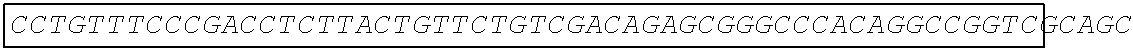 |
| | 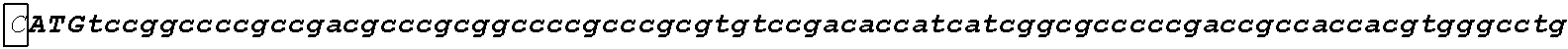 |
| | accatcctggtcgccctgctgctgtccgccttctgcttctgcgagttcttcatgctgcccgtggccatggccctgctgcccgtgcccggcacca |
| | cctcccgcaagctgctgcgccgctgggagggcctggtggccaagacctggctgtccttcggcggctggctgctggagaacgtgggcggc |
| | gtgaagctgatcctgacgggcgactccatcctgcccggcgaccgcgtgctgatcctgtccaaccaccgcacccgcatcgactggatgttc |
| | ctgtggtgctgggccgcccgcttcgacctgctgtcctcctaccgcgtgatcctgaagtcctccctgcgcaccttcccctggtggggctgggg |
| | catgtccctgtgcctgttccccttcatccaccgcggctccaacaaccgcgacgccgacctgacccgcatcgagcacatctgccagtacctg |
| | tccgagctgggcgtgcccaactccctgatcctgttccccgagggcaccgacctgtcccagtccaaccagaagcgcgaccgcgactacgc |
| | cctggccaagaacctgcccatctaccacaacgtgctgcacccccgctccggcgccttcatcgcctccctgaccgccatgcgcccccacct |
| | ggacgccgtggtggacctgaccatcggctacgtggactacacccccggcgagcgcccctccgagctgtccctgctgaagggccgcctg |
| | ccccacgaggtgcacatcaacatgaagcgctgggacatcaagaccacccccctgctgcgcgagaacccctccgaccgcgccgagca |
| | gttcctgcgcgagtccttcgaccgcaaggaggccctgctgaccgccttctactccaacaacaccgacacctccggcggcggcctgaagc |
| | agacctcctccctgctggtgtcctcctcctcctcccccttctccaactcctccgcccccctgggcttcccctccttctcctccccctcgacctccg |
| | tggcctcctcctacaacgagcccgtgtacgtgcacaacgcccactggacctacggcaagggcctgctgggcacgggcatcgccatggc |
| | cctcgtgctgatgctggcctgctgcctgcccgccgtggccttctgcggctactccatcgtggtgttcctgctgttcgtgaccgtgaactccgtgt |
| | gggagggcttcgacaccctggagctgaacctggccgtgtggtggggggccaggcgggccagaccaagcaggagTAGttaattaa |
| | gcagcagcagctcggatagtatcgacacactctggacgctggtcgtgtgatggactgttgccgccacacttgctgccttgacctgtgaatatcc |
| | ctgccgcttttatcaaacagcctcagtgtgtttgatcttgtgtgtacgcgcttttgcgagttgctagctgcttgtgctatttgcgaataccacccccag |
| | catccccttccctcgtttcatatcgcttgcatcccaaccgcaacttatctacgctgtcctgctatccctcagcgctgctcctgctcctgctcactgcc |
| | cctcgcacagccttggtttgggctccgcctgtattctcctggtactgcaacctgtaaaccagcactgcaatgctgatgcacgggaagtagtggg |
| | atgggaacacaaatggaagcgtctgcgtgttgggagctggagtcgtgggcttgacgacggcgctgcagctcttgcaggatgtgcctgg |
| | cgtgcgcgttcacgtcgtggctgagaaatatggcgacgaaacgttgacggctggggccggcgggctgtggatgccatacgcattggg |
| | tacgcggccattggatgggattggtaggcttatggagggataatagagtttttgtcggatccaacgcatgttgatgcagtagcccggtg |
| | tgctgaaagtatggaaggatggtgcattggctattcacatgcgctgcccaccccttttcgcaggaaatgtgccggcatcgttggtgcac |
| | cgatggggaaaatcgacgttcgaccactacatgaaaatgtatacctctgaagatgcagcaactgcgggtgcgaaacggataacggt |
| | ttggtaggtgtatgtcgcagcatgtgctggattttgcgggcaactccccctgccacggcccattgcaggtgtcatgttgactggaggat |
| | acgaactttcggccgtcaaattcccagaggaggaccccctctgggccgacattgtgcccactgctcttccgcttgctgtttcctgtgtga |
| | aattgt |

The construct can be written as 5′DAO1b::CrTUB2:ScSUC2:PmPGH:CvNR:PmAMT03:NoLPAT4:CvNR::3′DAO1b. Proceeding in the 5′ to 3′ direction, bold, lowercase sequences represent genomic DNA from CHK22 that permit targeted integration at the DAO1b locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text followed by a linker, indicated by lowercase, bold italics. The P. moriformis PmAMT03 promoter, indicated by boxed italicized text, drives the expression of the NoLPAT4. The initiator ATG and terminator TGA codons of the NoLPAT4 are indicated by uppercase, bold italics, while the remainder of the gene is indicated by bold italics. The C. vulgaris nitrate reductase 3′-UTR is indicated by lowercase underlined text followed by the CHK22 DAO1b genomic region indicated by bold, lowercase text.

Table 9 shows the fatty acid/TAG profiles of four primary transformants of CHK22 transformed with pCHK386. P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 9
Screen of primary transformants of CHK22 with pCHK386.

| | FAME Profiles | | | | | | | | | TAG Profile |
|---|---|---|---|---|---|---|---|---|---|---|
| | | | | | | | C18:3 | | | m/z 577/603 |
| Sample | C12:0 | C14:0 | C16:0 | C18:0 | C18:1 | C18:2 | alpha | C20:0 | C20:1 | (OP:OO) |
| pCHK386-1 | 0.03 | 1.18 | 21.97 | 3.22 | 61.19 | 10.16 | 0.60 | 0.28 | 0.13 | 1.50 |
| pCHK386-2 | 0.04 | 1.18 | 20.89 | 3.36 | 61.46 | 10.84 | 0.57 | 0.27 | 0.13 | 1.46 |
| pCHK386-3 | 0.04 | 1.15 | 20.68 | 3.57 | 61.62 | 10.74 | 0.59 | 0.28 | 0.13 | 1.55 |
| pCHK386-4 | 0.04 | 1.18 | 20.80 | 3.48 | 61.42 | 10.86 | 0.58 | 0.27 | 0.13 | 1.53 |
| CHK22 | 0.03 | 1.21 | 26.75 | 3.40 | 59.41 | 7.11 | 0.52 | 0.28 | 0.10 | 1.54 |
| Control | | | | | | | | | | |

### Example 6: Expressing Synechocystis sp SII1848 in P. moriformis Strain CHK22

Synechocystis sp Sll1848 was introduced into a P. moriformis strain CHK22. The expression construct pCHK387 contained 5Y and 3Y homology arms to permit targeted integration of Sll1848 into the genome and is shown in Table 10.

TABLE 10
Integrative sequences for the transformation of P. moriformis with pCHK387
encoding Synechocystis sp SII1848.

| SEQ | |
|---|---|
| ID | |
| NO: | Sequence |
| 5 | gctcttccgcttgcccgcaccctcgttgatctgggagccctgcgcagccccttaaatcatctcagtcaggtttctatgttcaactgagcct |
| | aaagggctttcgtcacgcgcacgagcacacgtatatcggctacgcagtctctcagaagcggtagaacagttcgcaagccctcgtcgg |
| | tcgaaaacttgcgccagtactattgaattaaattaaatgatcgaatgagacgcgaaacttttgcagaatgccactgagtttgcccaga |
| | gaatgggagtggcgccattcaccatccgcctgtgcacggcctgattcgccgagacgatgaatggcgagaccagggagcggcttgcg |
| | agccccgagccggtagcaggaataacggtcgacaatcttcctgtccaattactggcaaccattagaaagagccggagcgcgttgaa |
| | attctgcaatcgagtaatttttcgatgcggcgggcctgctgaaccctaaggctccggcctatgtttaaggcgatccaagatgcacgcgg |
| | ccccaggcacgtgtctcaagcacaaaccccagccttagtttcgagactttgggagatagcggcagatatctagtttggcattttgtatat |
| | taattaagtctcgcaatggagcgctctgatgcggtgcagcgtcggctgcagcacctggcagtggcgctggggtcgccctatcgctcgg |
| | 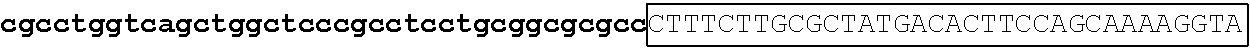 |
| | 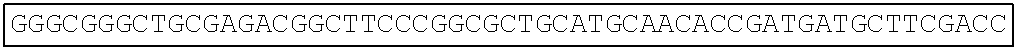 |
| | 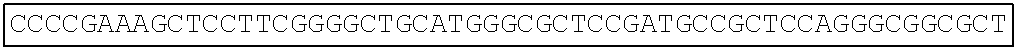 |
| | 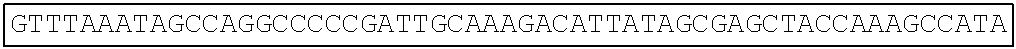 |
| | 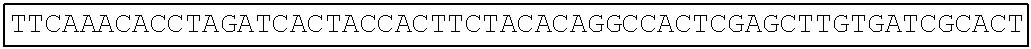 |
| | 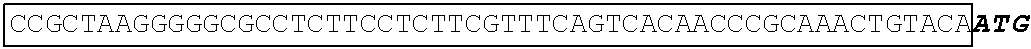 |
| | ctgctgcaggccttcctgttcctgctggccggcttcgccgccaagatcagcgcctccatgacgaacgagacgtccgaccgccccctggtg |
| | cacttcacccccaacaagggctggatgaacgaccccaacggcctgtggtacgacgagaaggacgccaagtggcacctgtacttccagt |
| | acaacccgaacgacaccgtctgggggacgcccttgttctggggccacgccacgtccgacgacctgaccaactgggaggaccagccca |
| | tcgccatcgccccgaagcgcaacgactccggcgccttctccggctccatggtggtggactacaacaacacctccggcttcttcaacgaca |
| | ccatcgacccgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctggtacgagc |
| | cctcccagaagtggatcatgaccgcggccaagtcccaggactacaagatcgagatctactcctccgacgacctgaagtcctggaagctg |
| | gagtccgcgttcgccaacgagggcttcctcggctaccagtacgagtgccccggcctgatcgaggtccccaccgagcaggaccccagca |
| | agtcctactgggtgatgttcatctccatcaaccccggcgccccggccggcggctccttcaaccagtacttcgtcggcagcttcaacggcac |
| | ccacttcgaggccttcgacaaccagtcccgcgtggtggacttcggcaaggactactacgccctgcagaccttcttcaacaccgacccgac |
| | ctacgggagcgccctgggcatcgcgtgggcctccaactgggagtactccgccttcgtgcccaccaacccctggcgctcctccatgtccctc |
| | gtgcgcaagttctccctcaacaccgagtaccaggccaacccggagacggagctgatcaacctgaaggccgagccgatcctgaacatc |
| | agcaacgccggcccctggagccggttcgccaccaacaccacgttgacgaaggccaacagctacaacgtcgacctgtccaacagcacc |
| | ggcaccctggagttcgagctggtgtacgccgtcaacaccacccagacgatctccaagtccgtgttcgcggacctctccctctggttcaagg |
| | gcctggaggaccccgaggagtacctccgcatgggcttcgaggtgtccgcgtcctccttcttcctggaccgcgggaacagcaaggtgaag |
| | ttcgtgaaggagaacccctacttcaccaaccgcatgagcgtgaacaaccagcccttcaagagcgagaacgacctgtcctactacaagg |
| | tgtacggcttgctggaccagaacatcctggagctgtacttcaacgacggcgacgtcgtgtccaccaacacctacttcatgaccaccggga |
| | acgccctgggctccgtgaacatgacgacgggggtggacaacctgttctacatcgacaagttccaggtgcgcgaggtcaagtgactTA |
| | AGACGCCCGCGCGGCGCACCTGACCTGTTCTCTCGAGGGCGCCTGTTCTGCCTTGCGA |
| | AACAAGCCCCTGGAGCATGCGTGCATGATCGTCTCTGGCGCCCCGCCGCGCGGTTTGT |
| | CGCCCTCGCGGGCGCCGCGGCCGCGGGGGCGCATTGAAATTGTTGCAAACCCCACCT |
| | GACAGATTGAGGGCCCAGGCAGGAAGGCGTTGAGATGGAGGTACAGGAGTCAAGTA |
| | ACTGAAAGTTTTTATGATAACTAACAACAAAGGGTCGTTTCTGGCCAGCGAATGACA |
| | AGAACAAGATTCCACATTTCCGTGTAGAGGCTTGCCATCGAATGTGAGCGGGCGGGC |
| | CGCGGACCCGACAAAACCCTTACGACGTGGTAAGAAAAACGTGGCGGGCACTGTCCC |
| | TGTAGCCTGAAGACCAGCAGGAGACGATCGGAAGCATCACAGCACAACCGGTgcagcag |
| | cagctcggatagtatcgacacactctggacgctggtgtgtgatggactgttgccgccacacttgctgccttgacctgtgaatatccctgcc |
| | gcttttatcaaacagcctcagtgtgtttgatcttgtgtgtacgcgcttttgcgagttgctagctgcttgtgctatttgcgaataccacccccagca |
| | tccccttccctcgtttcatatcgcttgcatcccaaccgcaacttatctacgctgtcctgctatccctcagcgctgctcctgctcctgctcactgcc |
| | cctcgcacagccttggtttgggctccgcctgtattctcctggtactgcaacctgtaaaccagcactgcaatgctgatgcacgggaagtagtg |
| | 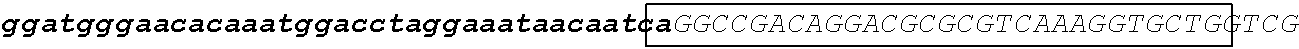 |
| | 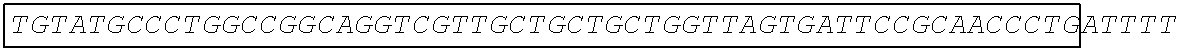 |
| | 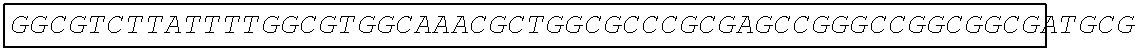 |
| | 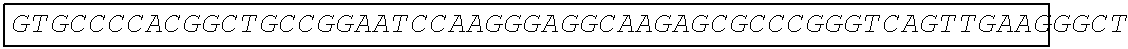 |
| | 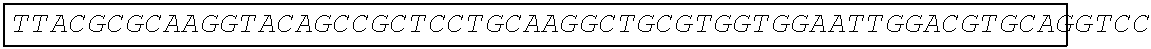 |
| | 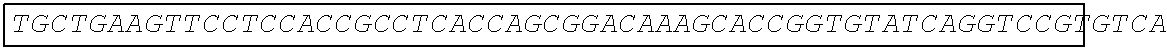 |
| | 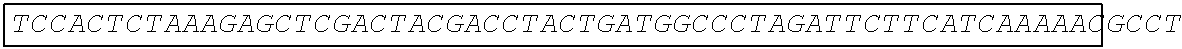 |
| | 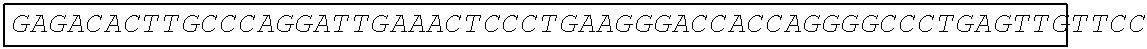 |
| | 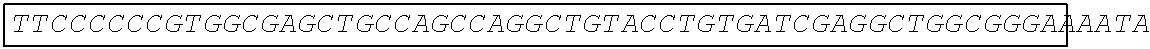 |
| | 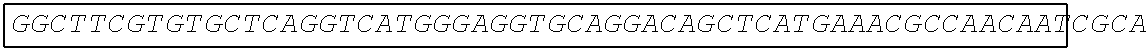 |
| | 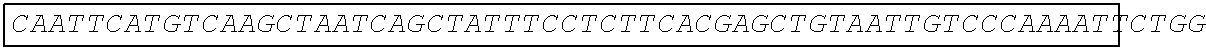 |
| | 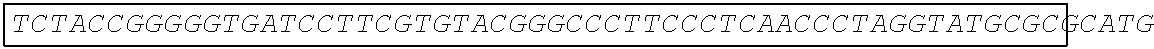 |
| | 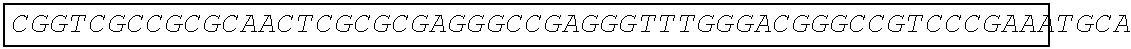 |
| | 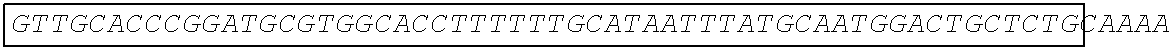 |
| | 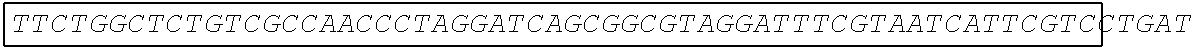 |
| | 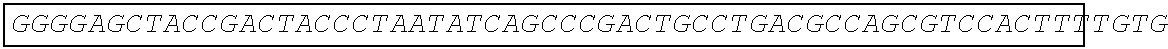 |
| | 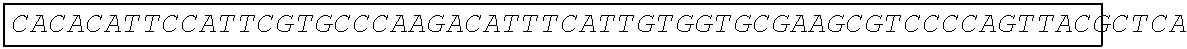 |
| | 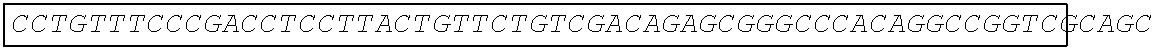 |
| | 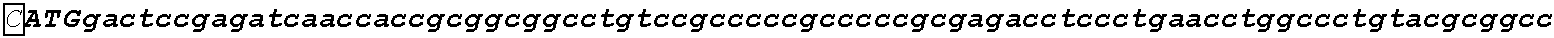 |
| | tgaagtggggcgtggtgcgccccctgctgcacggcctgttccaggcccaggtgtacggccaggagctggtgcccacccgcggccccgc |
| | cctggtggtgtccaaccacgcctcctacttcgacccccccttcctgtcctgcgccatggcccgccccgtggccttcatggccaaggaggag |
| | ctgttcaacgtgcccctgctgggccccgccatccgcctgtacggcgcctaccccgtgaagcgcggctccggcgaccgcggcgccctgcg |
| | cgccgccctgaccgccctgggcgacggctggctggtgggcgtgttcctggagggcacccgcaccaaggacggccgcatccaccagcc |
| | caagctgggcgccgccatgatcgccgccaaggcccaggtgcccatcatccccgtgtccctgggcggcgtggagcagatcttccagccc |
| | ggctccccctggccccaccccgtgcccctgaccatccgcatcggcaaggccatcgccccccccgtgaagaaccgcaagcccgagctgg |
| | aggccgtgaccaaggcctgccaggcccagatccacgagatgctggacctgggccgcgacTAGttaattaagcagcagcagctcgga |
| | tagtatcgacacactctggacgctggtcgtgtgatggactgttgccgccacacttgctgccttgacctgtgaatatccctgccgcttttatcaaac |
| | agcctcagtgtgtttgatcttgtgtgtacgcgcttttgcgagttgctagctgcttgtgctatttgcgaataccacccccagcatccccttccctcgttt |
| | catatcgcttgcatcccaaccgcaacttatctacgctgtcctgctatccctcagcgctgctcctgctcctgctcactgcccctcgcacagccttgg |
| | tttgggctccgcctgtattctcctggtactgcaacctgtaaaccagcactgcaatgctgatgcacgggaagtagtgggatgggaacacaaatgg |
| | aagcgtctgcgtgttgggagctggagtcgtgggcttgacgacggcgctgcagctcttgcaggatgtgcctggcgtgcgcgttcacgtc |
| | gtggctgagaaatatggcgacgaaacgttgacggctggggccggcgggctgtggatgccatacgcattgggtacgcggccattgga |
| | tgggattggtaggcttatggagggataatagagtttttgtcggatccaacgcatgttgatgcagtagcccggtgtgctgaaagtatgga |
| | aggatggtgcattggctattcacatgcgctgcccaccccttttcgcaggaaatgtgccggcatcgttggtgcaccgatggggaaaatc |
| | gacgttcgaccactacatgaaaatgtatacctctgaagatgcagcaactgcgggtgcgaaacggataacggtttggtaggtgtatgtc |
| | gcagcatgtgctggattttgcgggcaactccccctgccacggcccattgcaggtgtcatgttgactggaggatacgaactttcggccgt |
| | caaattcccagaggaggaccccctctgggccgacattgtgcccactgctcttccgcttgctgtttcctgtgtgaaattgt |

The construct can be written as 5′DAO1b::CrTUB2:ScSUC2:PmPGH:CvNR:PmAMT03:Sll1848:CvNR::3′DAO1b. Proceeding in the 5′ to 3′ direction, bold, lowercase sequences represent genomic DNA from CHK22 that permit targeted integration at the DAO1b locus via homologous recombination, the C. reinhwdiii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text followed by a linker, indicated by lowercase, bold italics. The P. moriformis PmAMT03 promoter, indicated by boxed italicized text, drives the expression of the Sll1848. The initiator ATG and terminator TGA codons of the Sll1848 are indicated by uppercase, bold italics, while the remainder of the gene is indicated by bold italics. The C. vulgaris nitrate reductase 3′-UTR is indicated by lowercase underlined text followed by the CHK22 DAO1b genomic region indicated by bold, lowercase text.

Table 11 shows the fatty acid/TAG profiles of four primary transformants of CHK22 transformed with pCHK387. P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 11
Screen of primary transformants of CHK22 with pCHK387.

| | FAME Profiles | | | | | | | | | TAG Profile |
|---|---|---|---|---|---|---|---|---|---|---|
| | | | | | | | C18:3 | | | m/z 577/603 |
| Sample | C12:0 | C14:0 | C16:0 | C18:0 | C18:1 | C18:2 | alpha | C20:0 | C20:1 | (OP:OO) |
| pCHK387-1 | 0.03 | 0.81 | 19.20 | 2.44 | 60.34 | 14.56 | 0.76 | 0.27 | 0.23 | 1.60 |
| pCHK387-2 | 0.00 | 0.24 | 16.85 | 5.74 | 63.56 | 4.63 | 2.04 | 0.07 | 0.18 | 1.60 |
| pCHK387-3 | 0.04 | 0.73 | 17.67 | 2.02 | 58.43 | 18.37 | 0.84 | 0.22 | 0.25 | 1.50 |
| pCHK387-4 | 0.04 | 0.83 | 19.40 | 2.43 | 60.13 | 14.54 | 0.74 | 0.27 | 0.23 | 1.55 |
| CHK22 | 0.03 | 1.21 | 26.75 | 3.40 | 59.41 | 7.11 | 0.52 | 0.28 | 0.10 | 1.54 |
| Control | | | | | | | | | | |

### Example 7: Expressing Escherichia coli EcPlsC in P. moriformis Strain CHK22

The E. coli EcPlsC was introduced into a P. moriformis strain CHK22. The expression construct pCHK388 contained 5′ and 3′ homology arms to permit targeted integration of EcPlsC into the genome and is shown in Table 12.

TABLE 12
Integrative sequences for the transformation of P. moriformis with pCHK388
encoding Escherichia coli EcPlsC.

| SEQ ID | |
|---|---|
| NO: | Sequence |
| 6 | gctcttccgcttgcccgcaccctcgttgatctgggagccctgcgcagccccttaaatcatctcagtcaggtttctatgttcaactgagcct |
| | aaagggctttcgtcacgcgcacgagcacacgtatatcggctacgcagtctctcagaagcggtagaacagttcgcaagccctcgtcgg |
| | tcgaaaacttgcgccagtactattgaattaaattaaatgatcgaatgagacgcgaaacttttgcagaatgccactgagtttgcccaga |
| | gaatgggagtggcgccattcaccatccgcctgtgcacggcctgattcgccgagacgatgaatggcgagaccagggagcggcttgcg |
| | agccccgagccggtagcaggaataacggtcgacaatcttcctgtccaattactggcaaccattagaaagagccggagcgcgttgaa |
| | attctgcaatcgagtaatttttcgatgcggcgggcctgctgaaccctaaggctccggcctatgtttaaggcgatccaagatgcacgcgg |
| | ccccaggcacgtgtctcaagcacaaaccccagccttagtttcgagactttgggagatagcggcagatatctagtttggcattttgtatat |
| | taattaagtctcgcaatggagcgctctgatgcggtgcagcgtcggctgcagcacctggcagtggcgctggggtcgccctatcgctcgg |
| | 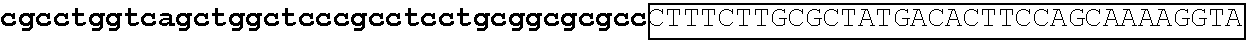 |
| | 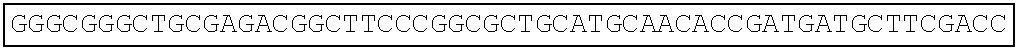 |
| | 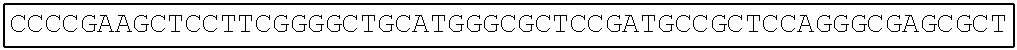 |
| | 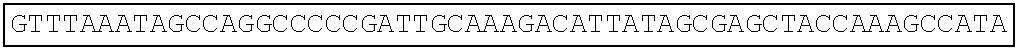 |
| | 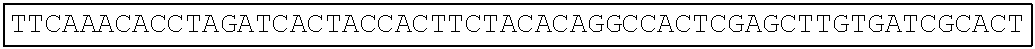 |
| | 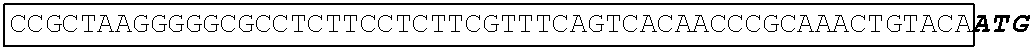 |
| | ctgctgcaggccttcctgttcctgctggccggcttcgccgccaagatcagcgcctccatgacgaacgagacgtccgaccgccccctggtg |
| | cacttcacccccaacaagggctggatgaacgaccccaacggcctgtggtacgacgagaaggacgccaagtggcacctgtacttccagt |
| | acaacccgaacgacaccgtctgggggacgcccttgttctggggccacgccacgtccgacgacctgaccaactgggaggaccagccca |
| | tcgccatcgccccgaagcgcaacgactccggcgccttctccggctccatggtggtggactacaacaacacctccggcttcttcaacgaca |
| | ccatcgacccgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctggtacgagc |
| | cctcccagaagtggatcatgaccgcggccaagtcccaggactacaagatcgagatctactcctccgacgacctgaagtcctggaagctg |
| | gagtccgcgttcgccaacgagggcttcctcggctaccagtacgagtgccccggcctgatcgaggtccccaccgagcaggaccccagca |
| | agtcctactgggtgatgttcatctccatcaaccccggcgccccggccggcggctccttcaaccagtacttcgtcggcagcttcaacggcac |
| | ccacttcgaggccttcgacaaccagtcccgcgtggtggacttcggcaaggactactacgccctgcagaccttcttcaacaccgacccgac |
| | ctacgggagcgccctgggcatcgcgtgggcctccaactgggagtactccgccttcgtgcccaccaacccctggcgctcctccatgtccctc |
| | gtgcgcaagttctccctcaacaccgagtaccaggccaacccggagacggagctgatcaacctgaaggccgagccgatcctgaacatc |
| | agcaacgccggcccctggagccggttcgccaccaacaccacgttgacgaaggccaacagctacaacgtcgacctgtccaacagcacc |
| | ggcaccctggagttcgagctggtgtacgccgtcaacaccacccagacgatctccaagtccgtgttcgcggacctctccctctggttcaagg |
| | gcctggaggaccccgaggagtacctccgcatgggcttcgaggtgtccgcgtcctccttcttcctggaccgcgggaacagcaaggtgaag |
| | ttcgtgaaggagaacccctacttcaccaaccgcatgagcgtgaacaaccagcccttcaagagcgagaacgacctgtcctactacaagg |
| | tgtacggcttgctggaccagaacatcctggagctgtacttcaacgacggcgacgtcgtgtccaccaacacctacttcatgaccaccggga |
| | acgccctgggctccgtgaacatgacgacgggggtggacaacctgttctacatcgacaagttccaggtgcgcgaggtcaagtgactTA |
| | AGACGCCCGCGCGGCGCACCTGACCTGTTCTCTCGAGGGCGCCTGTTCTGCCTTGCGA |
| | AACAAGCCCCTGGAGCATGCGTGCATGATCGTCTCTGGCGCCCCGCCGCGCGGTTTGT |
| | CGCCCTCGCGGGCGCCGCGGCCGCGGGGGCGCATTGAAATTGTTGCAAACCCCACCT |
| | GACAGATTGAGGGCCCAGGCAGGAAGGCGTTGAGATGGAGGTACAGGAGTCAAGTA |
| | ACTGAAAGTTTTTATGATAACTAACAACAAAGGGTCGTTTCTGGCCAGCGAATGACA |
| | AGAACAAGATTCCACATTTCCGTGTAGAGGCTTGCCATCGAATGTGAGCGGGCGGGC |
| | CGCGGACCCGACAAAACCCTTACGACGTGGTAAGAAAAACGTGGCGGGCACTGTCCC |
| | TGTAGCCTGAAGACCAGCAGGAGACGATCGGAAGCATCACAGCACAACCGGTgcagcag |
| | cagctcggatagtatcgacacactctggacgctggtcgtgtgatggactgttgccgccacacttgctgccttgacctgtgaatatccctgcc |
| | gcttttatcaaacagcctcagtgtgtttgatcttgtgtgtacgcgcttttgcgagttgctagctgcttgtgctatttgcgaataccacccccagca |
| | tccccttccctcgtttcatatcgcttgcatcccaaccgcaacttatctacgctgtcctgctatccctcagcgctgctcctgctcctgctcactgcc |
| | cctcgcacagccttggtttgggctccgcctgtattctcctggtactgcaacctgtaaaccagcactgcaatgctgatgcacgggaagtagtg |
| | 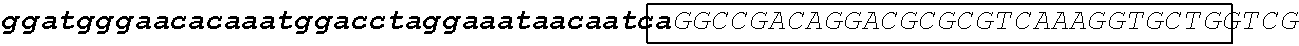 |
| | 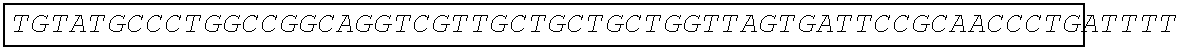 |
| | 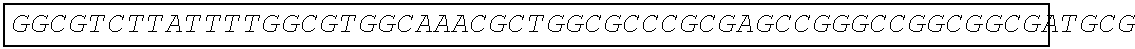 |
| | 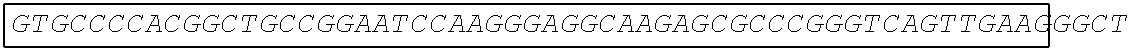 |
| | 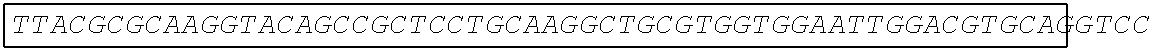 |
| | 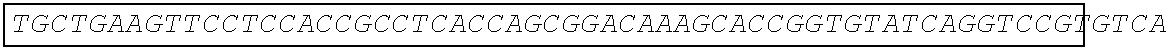 |
| | 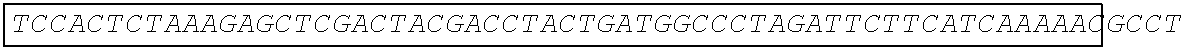 |
| | 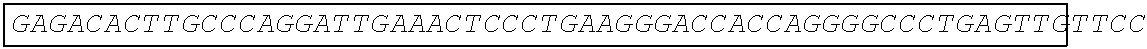 |
| | 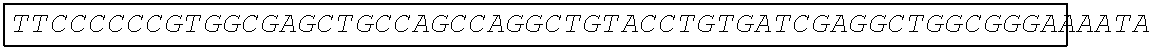 |
| | 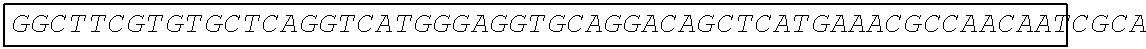 |
| | 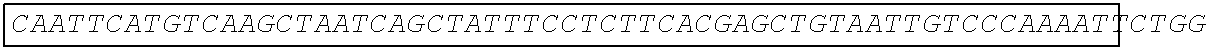 |
| | 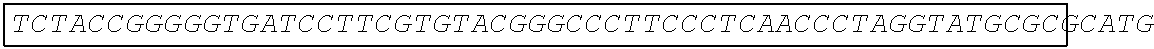 |
| | 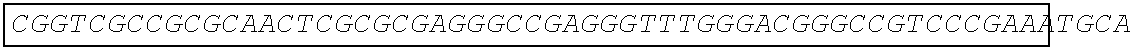 |
| | 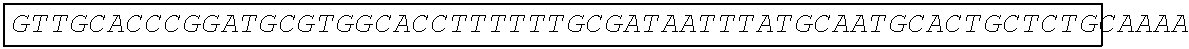 |
| | 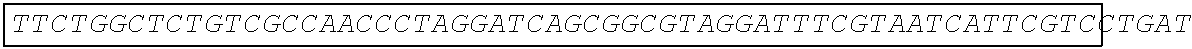 |
| | 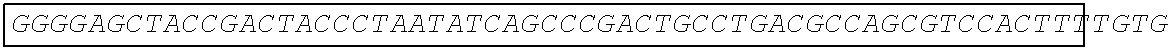 |
| | 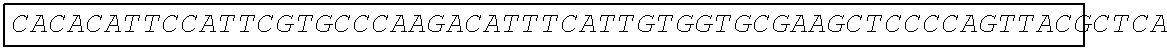 |
| | 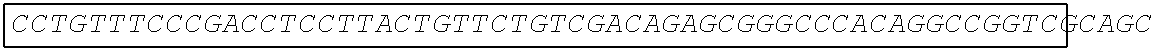 |
| | 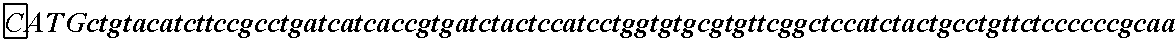 |
| | ccccaagcacgtcgccaccttcggccacatgttcggccgcctggcccccctgttcggcctgaaggtggagtgccgcaagcccaccgacg |
| | ccgagtcctacggcaacgccatctacatcgccaaccaccagaacaactacgacatggtgaccgcctccaacatcgtgcagccccccac |
| | cgtgaccgtgggcaagaagtccctgctgtggattcccttcttcggccagctctactggctgacgggcaacctgctgatcgaccgcaacaa |
| | ccgcaccaaggcccacggcaccatcgccgaggtggtcaaccacttcaagaagcgccgcatctccatctggatgttccccgagggcacc |
| | cgctcccgcggccggggcctgctgcccttcaagacgggcgccttccacgccgccatcgccgcgggcgtgcccatcatccccgtgtgcgt |
| | gtccaccacctccaacaagatcaacctgaaccgcctgcacaacggcctggtgatcgtggagatgctgccccccatcgacgtgtcccagt |
| | acggcaaggaccaggtgcgcgagctggccgcccactgccgctccatcatggagcagaagatcgccgagctggacaaggaggtggcc |
| | gagcgcgaggccgcgggcaaggtgTAGttaattaagcagcagcagctcggatagtatcgacacactctggacgctggtcgtgtgatgg |
| | actgttgccgccacacttgctgccttgacctgtgaatatccctgccgcttttatcaaacagcctcagtgtgtttgatcttgtgtgtacgcgcttttgcg |
| | agttgctagctgcttgtgctatttgcgaataccacccccagcatccccttccctcgtttcatatcgcttgcatcccaaccgcaacttatctacgctgt |
| | cctgctatccctcagcgctgctcctgctcctgctcactgcccctcgcacagccttggtttgggctccgcctgtattctcctggtactgcaacctgta |
| | aaccagcactgcaatgctgatgcacgggaagtagtgggatgggaacacaaatggaagcgtctgcgtgttgggagctggagtcgtgggctt |
| | gacgacggcgctgcagctcttgcaggatgtgcctggcgtgcgcgttcacgtcgtggctgagaaatatggcgacgaaacgttgacggc |
| | tggggccggcgggctgtggatgccatacgcattgggtacgcggccattggatgggattggtaggcttatggagggataatagagtttt |
| | tgtcggatccaacgcatgttgatgcagtagcccggtgtgctgaaagtatggaaggatggtgcattggctattcacatgcgctgcccac |
| | cccttttcgcaggaaatgtgccggcatcgttggtgcaccgatggggaaaatcgacgttcgaccactacatgaaaatgtatacctctga |
| | agatgcagcaactgcgggtgcgaaacggataacggtttggtaggtgtatgtcgcagcatgtgctggattttgcgggcaactccccctg |
| | ccacggcccattgcaggtgtcatgttgactggaggatacgaactttcggccgtcaaattcccagaggaggaccccctctgggccgac |
| | attgtgcccactgctcttccgcttgctgtttcctgtgtgaaattgt |

The construct can be written as 5′DAO1b::CrTUB2:ScSUC2:PmPGH:CvNR:PmAMT03:EcPlsC:CvNR::3′DAO1b. Proceeding in the 5′ to 3′ direction, bold, lowercase sequences represent genomic DNA from CHK22 that permit targeted integration at the DAO1b locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text followed by a linker, indicated by lowercase, bold italics. The P. moriformis PmAMT03 promoter, indicated by boxed italicized text, drives the expression of the EcPlsC. The initiator ATG and terminator TGA codons of the EcPlsC are indicated by uppercase, bold italics, while the remainder of the gene is indicated by bold italics. The C. vulgaris nitrate reductase 3′-UTR is indicated by lowercase underlined text followed by the CHK22 DAO1b genomic region indicated by bold, lowercase text.

Table 13 shows the fatty acid/TAG profiles of four primary transformants of CHK22 transformed with pCHK388. P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 13
Screen of primary transformants of CHK22 with pCHK388.

| | FAME Profiles | | | | | | | | | TAG Profile |
|---|---|---|---|---|---|---|---|---|---|---|
| | | | | | | | C18:3 | | | m/z 577/603 |
| Sample | C12:0 | C14:0 | C16:0 | C18:0 | C18:1 | C18:2 | alpha | C20:0 | C20:1 | (OP:OO) |
| pCHK388-1 | 0.03 | 1.19 | 24.48 | 3.30 | 60.71 | 8.20 | 0.53 | 0.27 | 0.11 | 1.55 |
| pCHK388-2 | 0.04 | 1.23 | 21.52 | 2.79 | 59.05 | 13.08 | 0.64 | 0.22 | 0.13 | 1.52 |
| pCHK388-3 | 0.04 | 1.20 | 21.59 | 2.81 | 59.43 | 12.62 | 0.62 | 0.22 | 0.13 | 1.54 |
| pCHK388-4 | 0.03 | 1.18 | 23.32 | 3.24 | 60.90 | 9.14 | 0.55 | 0.28 | 0.12 | 1.59 |
| CHK22 | 0.03 | 1.21 | 26.75 | 3.40 | 59.41 | 7.11 | 0.52 | 0.28 | 0.10 | 1.54 |
| Control | | | | | | | | | | |

### Example 8: Expressing Nannochloropsis NoLPAT3 in P. moriformis Strain CHK22

The Nannochloropsis NoLPAT3 was introduced into a P. moriformis strain CHK22. The expression construct pCHK453 contained 5′ and 3′ homology arms to permit targeted integration of NoLPAT3 into the genome and is shown in Table 14.

TABLE 14
Integrative sequences for the transformation of P. moriformis with pCHK453
encoding Nannochloropsis NoLPAT3.

| SEQ ID | |
|---|---|
| NO: | Sequence |
| 7 | ccctcaactgcgacgctgggaaccttctccgggcaggcgatgtgcgtgggtttgcctccttggcacggctctacaccgtcgagtacgcc |
| | atgaggcggtgatggctgtgtcggttgccacttcgtccagagacggcaagtcgtccatcctctgcgtgtgtggcgcgacgctgcagca |
| | gtccctctgcagcagatgagcgtgactttggccatttcacgcactcgagtgtacacaatccatttttcttaaagcaaatgactgctgatt |
| | gaccagatactgtaacgctgatttcgctccagatcgcacagatagcgaccatgttgctgcgtctgaaaatctggattccgaattcgacc |
| | ctggcgctccatccatgcaacagatggcgacacttgttacaattcctgtcacccatcggcatggagcaggtccacttagattcccgatc |
| | acccacgcacatctcgctaatagtcattcgttcgtgtcttcgatcaatctcaagtgagtgtgcatggatcttggttgacgatgcggtatg |
| | ggtttgcgccgctggctgcagggtctgcccaaggcaagctaacccagctcctctccccgacaatactctcgcaggcaaagccggtca |
| | cttgccttccagattgccaataaactcaattatggcctctgtcatgccatccatgggtctgatgaatggtcacgctcgtgtcctgaccgtt |
| | 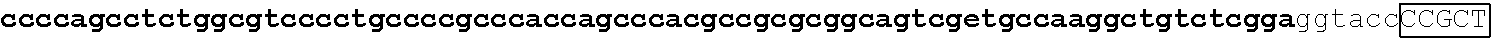 |
| | 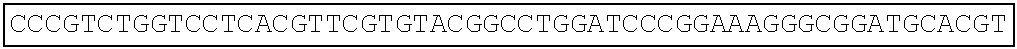 |
| | 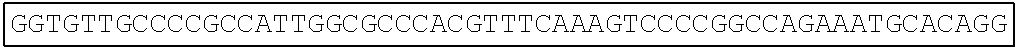 |
| | 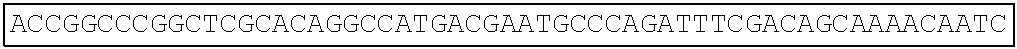 |
| | 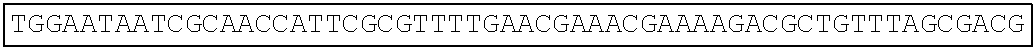 |
| | 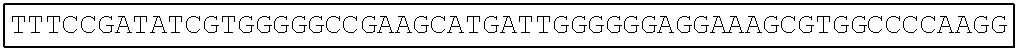 |
| | 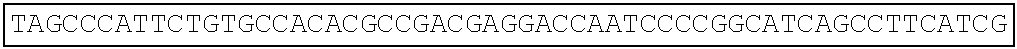 |
| | 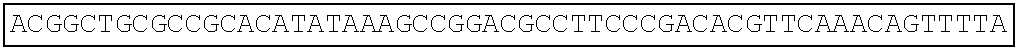 |
| | 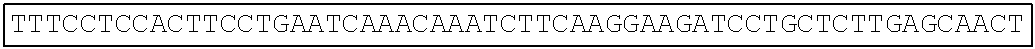 |
| | 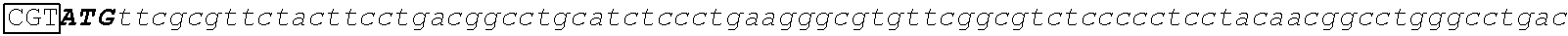 |
| | gccccagatgggctgggacaactggaacacgttcgcctgcgacgtctccgagcagctgctgctggacacggccgaccgcatctccgac |
| | ctgggcctgaaggacatgggctacaagtacatcatcctggacgactgctggtcctccggccgcgactccgacggcttcctggtcgccgac |
| | gagcagaagttccccaacggcatgggccacgtcgccgaccacctgcacaacaactccttcctgttcggcatgtactcctccgcgggcga |
| | gtacacgtgcgccggctaccccggctccctgggccgcgaggaggaggacgcccagttcttcgcgaacaaccgcgtggactacctgaa |
| | gtacgacaactgctacaacaagggccagttcggcacgcccgagatctcctaccaccgctacaaggccatgtccgacgccctgaacaag |
| | acgggccgccccatcttctactccctgtgcaactggggccaggacctgaccttctactggggctccggcatcgcgaactcctggcgcatgt |
| | ccggcgacgtcacggcggagttcacgcgccccgactcccgctgcccctgcgacggcgacgagtacgactgcaagtacgccggcttcca |
| | ctgctccatcatgaacatcctgaacaaggccgcccccatgggccagaacgcgggcgtcggcggctggaacgacctggacaacctgga |
| | ggtcggcgtcggcaacctgacggacgacgaggagaaggcgcacttctccatgtgggccatggtgaagtcccccctgatcatcggcgcg |
| | aacgtgaacaacctgaaggcctcctcctactccatctactcccaggcgtccgtcatcgccatcaaccaggactccaacggcatccccgcc |
| | acgcgcgtctggcgctactacgtgtccgacacggacgagtacggccagggcgagatccagatgtggtccggccccctggacaacggc |
| | gaccaggtcgtggcgctgctgaacggcggctccgtgtcccgccccatgaacacgaccctggaggagatcttcttcgactccaacctggg |
| | ctccaagaagctgacctccacctgggacatctacgacctgtgggcgaaccgcgtcgacaactccacggcgtccgccatcctgggccgc |
| | aacaagaccgccaccggcatcctgtacaacgccaccgagcagtcctacaaggacggcctgtccaagaacgacacccgcctgttcggc |
| | cagaagatcggctccctgtcccccaacgcgatcctgaacacgaccgtccccgcccacggcatcgcgttctaccgcctgcgcccctcctcc |
| | TGATACAACTTATTACGTATTCTGACCGGCGCTGATGTGGCGCGGACGCCGTCGTACT |
| | CTTTCAGACTTTACTCTTGAGGAATTGAACCTTTCTCGCTTGCTGGCATGTAAACATTG |
| | GCGCAATTAATTGTGTGATGAAGAAAGGGTGGCACAAGATGGATCGCGAATGTACGA |
| | GATCGACAACGATGGTGATTGTTATGAGGGGCCAAACCTGGCTCAATCTTGTCGCATG |
| | TCCGGCGCAATGTGATCCAGCGGCGTGACTCTCGCAACCTGGTAGTGTGTGCGCACCG |
| | GGTCGCTTTGATTAAAACTGATCGCATTGCCATCCCGTCAACTCACAAGCCTACTCTA |
| | GCTCCCATTGCGCACTCGGGCGCCCGGCTCGATCAATGTTCTGAGCGGAGGGCGAAG |
| | CGTCAGGAAATCGTCTCGGCAGCTGGAAGCGCATGGAATGCGGAGCGGAGATCGAAT |
| | 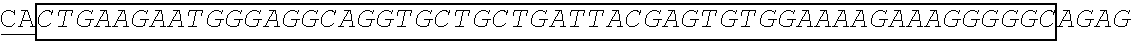 |
| | 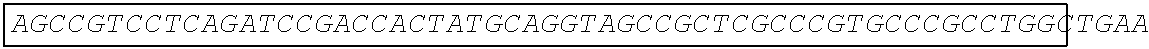 |
| | 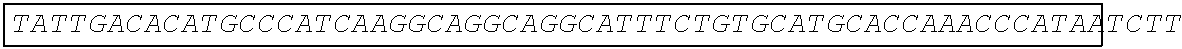 |
| | 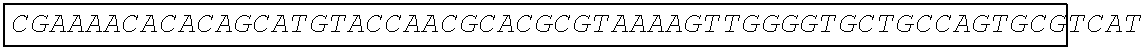 |
| | 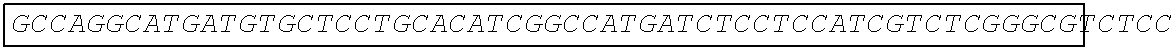 |
| | 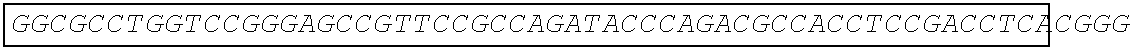 |
| | 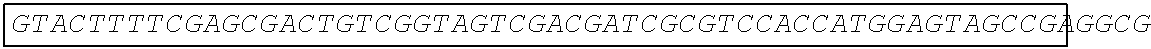 |
| | 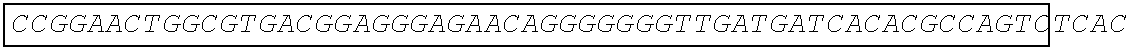 |
| | 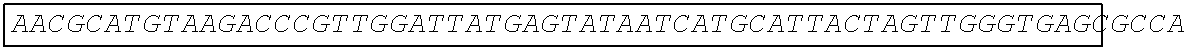 |
| | 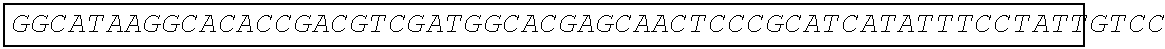 |
| | 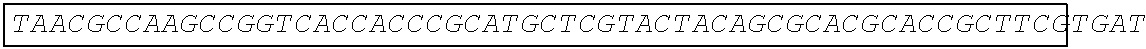 |
| | 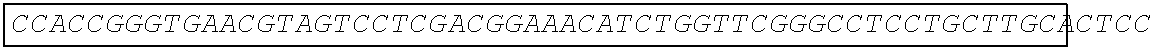 |
| | 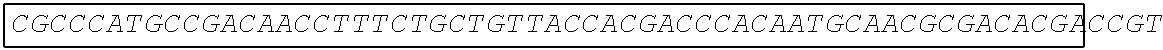 |
| | 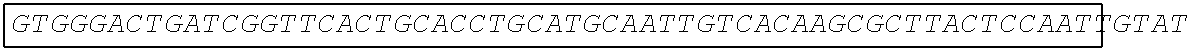 |
| | 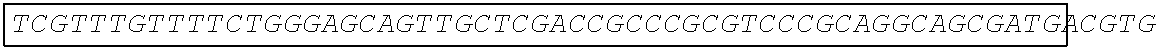 |
| | 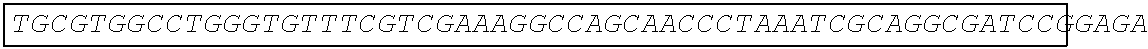 |
| | 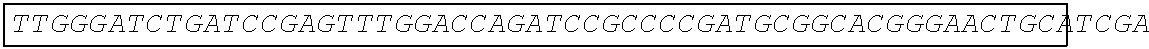 |
| | 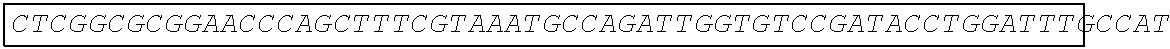 |
| | 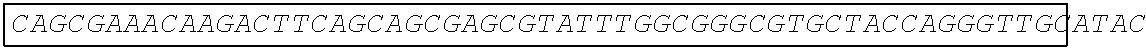 |
| | 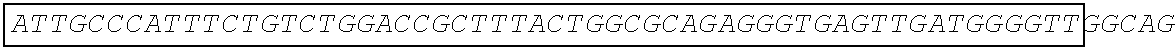 |
| | 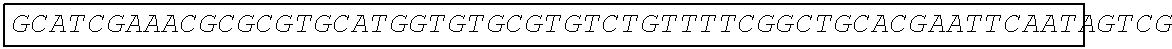 |
| | 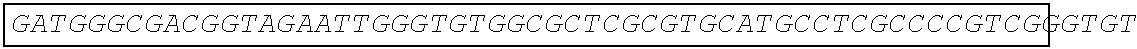 |
| | 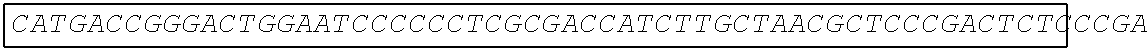 |
| | 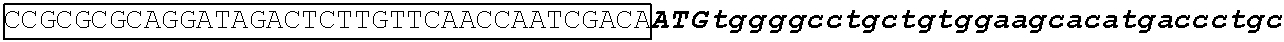 |
| | gcctgctgttcgtgtccgtgtgcctggtgacctcctccatcggcgcgaacatgatggccctggccctgttcctggtgatccgccccttctcccg |
| | ctccctgtaccgccgcctggtgtcccagtgcgtggcctgcatgtggatcgacgccctgtccctgctgctgcccggcaccaacatccacatc |
| | gccgccgactccgacatgcccgacggcatcaccgcgggcatcgtggtggccaaccaccagtacgagggcgactggtggttcatgctga |
| | tggtggcccgcttcctgggcctgcacggcaacgtgaagatcatcgtgcgcgagggcctgaagaagatccccctgctgggctggctggtg |
| | cgcctggtggagtaccccttcatctcctcctcctggtccctgtcccgcaccaacctgttcggcctgctgcgctccttcaacgccgacgacttcc |
| | ccgtgctgctgttccagttccccgagggcgaccgcatcgacgccaaggtgcgccagcagtccctggccttcgccgccaaggagcagcg |
| | cccccacctgctgcacgtgctgctgccccgcaccacgggcttcaacacctgcatcgaggccctgcgcacctcccacccccccgtgtacga |
| | catgaccatcgccatccccggcaccacgggccagccctcctcctcctcctcctcctcctccccctccgccgccgccgccgccgcctccccc |
| | tccgcgggcgccgccgccgccgccgccgccgcctccaacacctccaccaacccctcctccaaccccccctcctcctcctccaccgacac |
| | ctcccccgccgccgccgccgccgccgccgccgacgccgccgccgccgagtcccacgacgcctccctgttccacaccttcctgcgcttctg |
| | caacggcgagggcccccgcgacgtgcacatccgcctgaagcgctactccctgaacgacgtgctggccgacccccactggctggacac |
| | caagtgggccgagaaggaccgcgtgctgacctacttctcccgccacgcctgcttccccgcccccctgcccccccacatggccaacggcg |
| | gcggctccacccagacccacggcggccggggggcccccgcggccccgcgggctcccacgccgtggtgggcgccgcgggcaacgc |
| | ccagtacctgcgctccttcaactcccgcaagttcaaggccgagacctccttcctggccctggcccgcctgctgctgacccccctgtgcctgc |
| | ccctgctgctgctgatggcctgccccctgatgaccctgtacgtgtgcgtgtccaccgtgcgccgctggctgggcgaggagatcgtgttctcc |
| | cccggcggccggggagggcgacgccgtgtacgagtccgtgaactggtccgtgtgattaattaagcagcagcagctcg |
| | gatagtatcgacacactctggacgctggtcgtgtgatggactgttgccgccacacttgctgccttgacctgtgaatatccctgccgcttttatcaa |
| | acagcctcagtgtgtttgatcttgtgtgtacgcgcttttgcgagttgctagctgcttgtgctatttgcgaataccacccccagcatccccttccctcg |
| | tttcatatcgcttgcatcccaaccgcaacttatctacgctgtcctgctatccctcagcgctgctcctgctcctgctcactgcccctcgcacagcctt |
| | ggtttgggctccgcctgtattctcctggtactgcaacctgtaaaccagcactgcaatgctgatgcacgggaagtagtgggatgggaacacaaat |
| | ggaggccggcccgcgtctcgaacagagcgcggcggccgcatcgatacctgcaggacagcgccatgccacgccctttgatggcttcaag |
| | tacgattacggtgttggattgtgtgtttgttgcgtagtgtgcatggtttagaataatacacttgatttcttgctcacggcaatctcggcttgt |
| | ccgcaggttcaaccccatttcggagtctcaggtcagccgcgcaatgaccagccgctacttcaaggacttgcacgacaacgccgaggt |
| | gagctatgtttaggacttgattggaaattgtcgtcgacgcatattcgcgctccgcgacagcacccaagcaaaatgtcaagtgcgttcc |
| | gatttgcgtccgcaggtcgatgttgtgatcgtcggcgccggatccgccggtctgtcctgcgcttacgagctgaccaagcaccctgacgt |
| | ccgggtacgcgagctgagattcgattagacataaattgaagattaaacccgtagaaaaatttgatggtcgcgaaactgtgctcgattg |
| | caagaaattgatcgtcctccactccgcaggtcgccatcatcgagcagggcgttgctcccggcggcggcgcctggctggggggacagc |
| | tgttctcggccatgtgtgtacgtagaaggatgaatttcagctggttttcgttgcacagctgtttgtgcatgatttgtttcagactattgttga |
| | atgtttttagatttcttaggatgcatgatttgtctgcatgcgact |

The construct can be written as 5′Thi4::PmHXT1v2:ScMEL1:PmPGK:CvNR:PmSAD2-2:NoLPAT3:CvNR::3′Thi4. Proceeding in the 5′ to 3′ direction, bold, lowercase sequences represent genomic DNA from strain CHK22 that permit targeted integration at the Thi4 locus via homologous recombination. The P. moriformis hexose transporter 1 (HXT1 v2) promoter driving expression of the S. caribergensis melibiase (ScMEL1) is indicated by boxed uppercase text. The initiator ATG and terminator TAA for ScMEL1 are indicated by uppercase, bold italics while the coding region is indicated in lowercase italics. P. moriformis phosphoglucokinase (PGK) 3′-UTR is indicated by uppercase underlined text followed by a stearoyl ACP desaturase-2 (SAD2-2) promoter from P. moriformis, indicated by boxed italicized text, driving the expression of NoLPAT3. The initiator ATG and terminator TGA codons of NoLPAT3 are indicated by uppercase, bold italics, while the remainder of the gene is indicated by bold italics. The C. vulgaris nitrate reductase 3′-UTR is indicated by lowercase underlined text followed by the P. moriformis Thi4 3′ flanking region indicated by bold, lowercase text.

Table 15 shows the fatty acid/TAG profiles of four primary transformants of CHK22 transformed with pCHK453. P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 15
Screen of primary transformants of CHK22 with pCHK453.

| | FAME Profiles | | | | | | | | | TAG Profile |
|---|---|---|---|---|---|---|---|---|---|---|
| | | | | | | | C18:3 | | | m/z 577/603 |
| Sample | C12:0 | C14:0 | C16:0 | C18:0 | C18:1 | C18:2 | alpha | C20:0 | C20:1 | (OP:OO) |
| pCHK453-1 | 0.03 | 1.20 | 26.34 | 3.37 | 60.29 | 6.78 | 0.48 | 0.26 | 0.10 | 1.49 |
| pCHK453-2 | 0.03 | 1.24 | 26.48 | 3.48 | 60.05 | 6.75 | 0.49 | 0.26 | 0.09 | 1.60 |
| pCHK453-3 | 0.03 | 1.20 | 26.28 | 3.07 | 60.33 | 7.09 | 0.47 | 0.24 | 0.07 | 1.57 |
| pCHK453-4 | 0.03 | 1.28 | 26.77 | 3.37 | 59.81 | 6.77 | 0.49 | 0.25 | 0.07 | 1.51 |
| CHK22 | 0.03 | 1.21 | 26.75 | 3.40 | 59.41 | 7.11 | 0.52 | 0.28 | 0.10 | 1.54 |
| Control | | | | | | | | | | |

### Example 9: Expressing Volvox carteri LPAAT2 (VcLPAAT2) in P. moriformis Strain CHK22

V. carteri VcLPAAT2 was introduced into a P. moriformis strain CHK22. The expression construct pCHK785 contained 5′ and 3′ homology arms to permit targeted integration of VcLPAAT2 into the genome and is shown in Table 16.

TABLE 16
Integrative sequences for the transformation of P. moriformis with pCHK785
encoding VcLPAAT2.

| SEQ | |
|---|---|
| ID | |
| NO: | Sequence |
| 8 | gctcttccgcttgcccgcaccctcgttgatctgggagccctgcgcagccccttaaatcatctcagtcaggtttctatgttcaactgagcct |
| | aaagggctttcgtcacgcgcacgagcacacgtatatcggctacgcagtctctcagaagcggtagaacagttcgcaagccctcgtcgg |
| | tcgaaaacttgcgccagtactattgaattaaattaaatgatcgaatgagacgcgaaacttttgcagaatgccactgagtttgcccaga |
| | gaatgggagtggcgccattcaccatccgcctgtgcacggcctgattcgccgagacgatgaatggcgagaccagggagcggcttgcg |
| | agccccgagccggtagcaggaataacggtcgacaatcttcctgtccaattactggcaaccattagaaagagccggagcgcgttgaa |
| | attctgcaatcgagtaatttttcgatgcggcgggcctgctgaaccctaaggctccggcctatgtttaaggcgatccaagatgcacgcgg |
| | ccccaggcacgtgtctcaagcacaaaccccagccttagtttcgagactttgggagatagcggcagatatctagtttggcattttgtatat |
| | taattaagtctcgcaatggagcgctctgatgcggtgcagcgtcggctgcagcacctggcagtggcgctggggtcgccctatcgctcgg |
| | 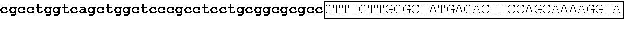 |
| | 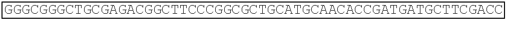 |
| | 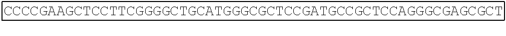 |
| | 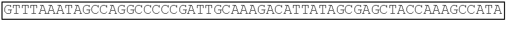 |
| | 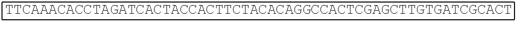 |
| | 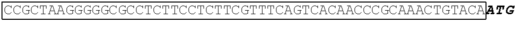 |
| | ctgctgcaggccttcctgttcctgctggccggcttcgccgccaagatcagcgcctccatgacgaacgagacgtccgaccgccccctggtg |
| | cacttcacccccaacaagggctggatgaacgaccccaacggcctgtggtacgacgagaaggacgccaagtggcacctgtacttccagt |
| | acaacccgaacgacaccgtctgggggacgcccttgttctggggccacgccacgtccgacgacctgaccaactgggaggaccagccca |
| | tcgccatcgccccgaagcgcaacgactccggcgccttctccggctccatggtggtggactacaacaacacctccggcttcttcaacgaca |
| | ccatcgacccgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctggtacgagc |
| | cctcccagaagtggatcatgaccgcggccaagtcccaggactacaagatcgagatctactcctccgacgacctgaagtcctggaagctg |
| | gagtccgcgttcgccaacgagggcttcctcggctaccagtacgagtgccccggcctgatcgaggtccccaccgagcaggaccccagca |
| | agtcctactgggtgatgttcatctccatcaaccccggcgccccggccggcggctccttcaaccagtacttcgtcggcagcttcaacggcac |
| | ccacttcgaggccttcgacaaccagtcccgcgtggtggacttcggcaaggactactacgccctgcagaccttcttcaacaccgacccgac |
| | ctacgggagcgccctgggcatcgcgtgggcctccaactgggagtactccgccttcgtgcccaccaacccctggcgctcctccatgtccctc |
| | gtgcgcaagttctccctcaacaccgagtaccaggccaacccggagacggagctgatcaacctgaaggccgagccgatcctgaacatc |
| | agcaacgccggcccctggagccggttcgccaccaacaccacgttgacgaaggccaacagctacaacgtcgacctgtccaacagcacc |
| | ggcaccctggagttcgagctggtgtacgccgtcaacaccacccagacgatctccaagtccgtgttcgcggacctctccctctggttcaagg |
| | gcctggaggaccccgaggagtacctccgcatgggcttcgaggtgtccgcgtcctccttcttcctggaccgcgggaacagcaaggtgaag |
| | ttcgtgaaggagaacccctacttcaccaaccgcatgagcgtgaacaaccagcccttcaagagcgagaacgacctgtcctactacaagg |
| | tgtacggcttgctggaccagaacatcctggagctgtacttcaacgacggcgacgtcgtgtccaccaacacctacttcatgaccaccggga |
| | acgccctgggctccgtgaacatgacgacgggggtggacaacctgttctacatcgacaagttccaggtgcgcgaggtcaagtgactTA |
| | AGACGCCCGCGCGGCGCACCTGACCTGTTCTCTCGAGGGCGCCTGTTCTGCCTTGCGA |
| | AACAAGCCCCTGGAGCATGCGTGCATGATCGTCTCTGGCGCCCCGCCGCGCGGTTTGT |
| | CGCCCTCGCGGGCGCCGCGGCCGCGGGGGCGCATTGAAATTGTTGCAAACCCCACCT |
| | GACAGATTGAGGGCCCAGGCAGGAAGGCGTTGAGATGGAGGTACAGGAGTCAAGTA |
| | ACTGAAAGTTTTTATGATAACTAACAACAAAGGGTCGTTTCTGGCCAGCGAATGACA |
| | AGAACAAGATTCCACATTTCCGTGTAGAGGCTTGCCATCGAATGTGAGCGGGCGGGC |
| | CGCGGACCCGACAAAACCCTTACGACGTGGTAAGAAAAACGTGGCGGGCACTGTCCC |
| | TGTAGCCTGAAGACCAGCAGGAGACGATCGGAAGCATCACAGCACAACCGGTgcagcag |
| | cagctcggatagtatcgacacactctggacgctggtcgtgtgatggactgttgccgccacacttgctgccttgacctgtgaatatccctgcc |
| | gcttttatcaaacagcctcagtgtgtttgatcttgtgtgtacgcgcttttgcgagttgctagctgcttgtgctatttgcgaataccacccccagca |
| | tccccttccctcgtttcatatcgcttgcatcccaaccgcaacttatctacgctgtcctgctatccctcagcgctgctcctgctcctgctcactgcc |
| | cctcgcacagccttggtttgggctccgcctgtattctcctggtactgcaacctgtaaaccagcactgcaatgctgatgcacgggaagtagtg |
| | 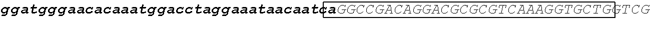 |
| | 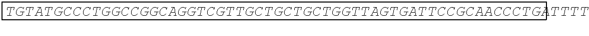 |
| | 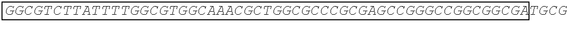 |
| | 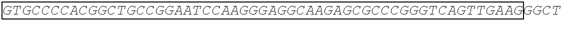 |
| | 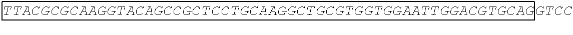 |
| | 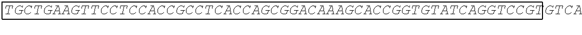 |
| | 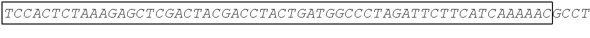 |
| | 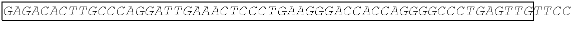 |
| | 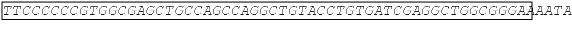 |
| | 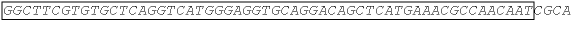 |
| | 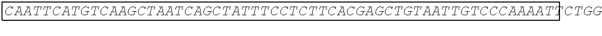 |
| | 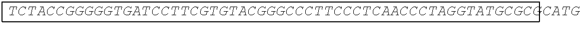 |
| | 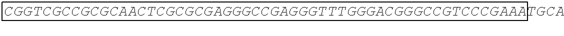 |
| | 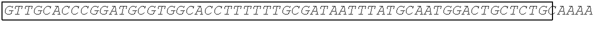 |
| | 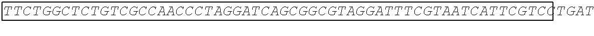 |
| | 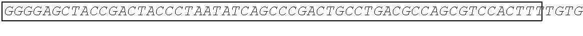 |
| | 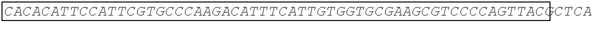 |
| | 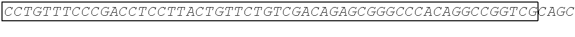 |
| | 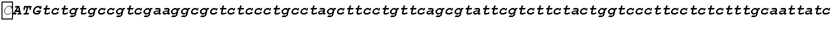 |
| | taccgcatccgtttcgctaacgtcggcaagcgcaacgacatgctcgactggtggatggtgtttttcgcgtttcctgtgttctgcacgtccgttc |
| | tgatcctgcggggtatcgtgctgtttaagcgcggacatatcgccgacaaggagttgctgaagtcggacgcggtggtcgtgatggtcgtgatgg |
| | ttgtggccaccgtcgtgcaggcgtttaacgtctggctggacgcaacgcttcgaaagtccaccgtccctggcctgctcgtgtacccggagggcc |
| | accgtagcctgaagccgcgcagcctgcccctcaagcggggaatgttgcacttcgctttcagccgccacctagccgtgcaactggtgatca |
| | cgcgtggtaaggaagaggtcatgtctgagaagacgtgctccgcgcggtggggccgcacccttgtcaccaactactcaaaggtgtgcgcc |
| | gggggccgctggggcggggtaatcaagccaacggatttcgactcgttcgacgcctttttcaacgagattcaaaccacgtgggacgcctg |
| | ctggcagctctcctaccaccagcccatcgcgggtgtcccccggctgtccctgaaggacgcgcacgagtacgactacccgccctccatgc |
| | gctggctgcaactgggcgtgaccctcctttcgatcgtgatcctcgcggccgtcctctacggcagttgggtcgtgctgcgcgcgggcgtgcta |
| | tcactgggctcctcggttacccaacaggcggtcgtggtcctcgggggcgccgggggctggctgggcctgtcgctgctccgcagcttcttaTAG |
| | ttaattaagcagcagcagctcggatagtatcgacacactctggacgctggtcgtgtgatggactgttgccgccacacttgctgccttgacctgtgaa |
| | tatccctgccgcttttatcaaacagcctcagtgtgtttgatcttgtgtgtacgcgcttttgcgagttgctagctgcttgtgctatttgcgaata |
| | ccacccccagcatccccttccctcgtttcatatcgcttgcatcccaaccgcaacttatctacgctgtcctgctatccctcagcgctgctcctgctcc |
| | tgctcactgcccctcgcacagccttggtttgggctccgcctgtattctcctggtactgcaacctgtaaaccagcactgcaatgctgatgcacggg |
| | aagtagtgggatgggaacacaaatggaagcgtctgcgtgttgggagctggagtcgtgggcttgacgacggcgctgcagctcttgcagg |
| | atgtgcctggcgtgcgcgttcacgtcgtggctgagaaatatggcgacgaaacgttgacggctggggccggcgggctgtggatgccat |
| | acgcattgggtacgcggccattggatgggattggtaggcttatggagggataatagagtttttgtcggatccaacgcatgttgatgca |
| | gtagcccggtgtgctgaaagtatggaaggatggtgcattggctattcacatgcgctgcccaccccttttcgcaggaaatgtgccggca |
| | tcgttggtgcaccgatggggaaaatcgacgttcgaccactacatgaaaatgtatacctctgaagatgcagcaactgcgggtgcgaaa |
| | cggataacggtttggtaggtgtatgtcgcagcatgtgctggattttgcgggcaactccccctgccacggcccattgcaggtgtcatgttg |
| | actggaggatacgaactttcggccgtcaaattcccagaggaggaccccctctgggccgacattgtgcccactgctcttccgcttgctgt |
| | ttcctgtgtgaaattgt |

The construct can be written as 5′DAO1b::CrTUB2:ScSUC2:PmPGH:CvNR:PmAMT03:VcLPAAT2:CvNR::3′DAO1b. Proceeding in the 5′ to 3′ direction, bold, lowercase sequences represent genomic DNA from CHK22 that permit targeted integration at the DAO1b locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text followed by a linker, indicated by lowercase, bold italics. The P. moriformis PmAMT03 promoter, indicated by boxed italicized text, drives the expression of the VcLPAAT2. The initiator ATG and terminator TGA codons of the VcLPAAT2 are indicated by uppercase, bold italics, while the remainder of the gene is indicated by bold italics. The C. vulgaris nitrate reductase 3′-UTR is indicated by lowercase underlined text followed by the CHK22 DAO1b genomic region indicated by bold, lowercase text.

Table 17 shows the fatty acid/TAG profiles of seven primary transformants of CHK22 transformed with pCHK785. The P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 17
Screen of primary transformants of CHK22 with pCHK785.

| | FAME Profiles | | | | | | | | | TAG Profile |
|---|---|---|---|---|---|---|---|---|---|---|
| | | | | | | | C18:3 | | | m/z 577/603 |
| Sample | C12:0 | C14:0 | C16:0 | C18:0 | C18:1 | C18:2 | alpha | C20:0 | C20:1 | (OP:OO) |
| pCHK785-1 | 0.06 | 1.60 | 21.48 | 3.63 | 57.66 | 12.96 | 0.56 | 0.30 | 0.23 | 1.55 |
| pCHK785-2 | 0.06 | 1.68 | 24.57 | 3.76 | 55.08 | 9.77 | 0.42 | 0.16 | 2.02 | 1.58 |
| pCHK785-3 | 0.05 | 1.61 | 25.61 | 2.89 | 56.40 | 8.44 | 0.43 | 0.29 | 1.80 | 1.51 |
| pCHK785-4 | 0.07 | 1.78 | 22.51 | 3.73 | 53.75 | 11.69 | 0.46 | 0.33 | 2.88 | 1.55 |
| pCHK785-5 | 0.08 | 1.99 | 24.84 | 3.29 | 53.55 | 10.28 | 0.46 | 0.24 | 2.69 | 1.51 |
| pCHK785-6 | 0.06 | 1.77 | 25.64 | 3.32 | 55.68 | 9.90 | 0.48 | 0.11 | 1.38 | 1.52 |
| pCHK785-7 | 0.04 | 1.52 | 25.33 | 3.06 | 58.60 | 8.97 | 0.47 | 0.27 | 0.25 | 1.58 |
| CHK22 | 0.04 | 1.52 | 26.43 | 2.97 | 58.76 | 7.88 | 0.44 | 0.25 | 0.23 | 1.53 |
| Control | | | | | | | | | | |

### Example 10: Expressing Chlamydomonase schloesseri LPAAT2 (ChsLPAAT2) in P. moriformis Strain CHK22

C. schloesseri ChsLPAAT2 was introduced into a P. moriformis strain CHK22. The expression construct pCHK786 contained 5Y and 3Y homology arms to permit targeted integration of ChsLPAAT2 into the genome and is shown in Table 18.

TABLE 18
Integrative sequences for the transformation of P. moriformis with pCHK786
encoding ChsLPAAT2.

| SEQ | |
|---|---|
| ID | |
| NO: | Sequence |
| 9 | gctcttccgcttgcccgcaccctcgttgatctgggagccctgcgcagccccttaaatcatctcagtcaggtttctatgttcaactgagcct |
| | aaagggctttcgtcacgcgcacgagcacacgtatatcggctacgcagtctctcagaagcggtagaacagttcgcaagccctcgtcgg |
| | tcgaaaacttgcgccagtactattgaattaaattaaatgatcgaatgagacgcgaaacttttgcagaatgccactgagtttgcccaga |
| | gaatgggagtggcgccattcaccatccgcctgtgcacggcctgattcgccgagacgatgaatggcgagaccagggagcggcttgcg |
| | agccccgagccggtagcaggaataacggtcgacaatcttcctgtccaattactggcaaccattagaaagagccggagcgcgttgaa |
| | attctgcaatcgagtaatttttcgatgcggcgggcctgctgaaccctaaggctccggcctatgtttaaggcgatccaagatgcacgcgg |
| | ccccaggcacgtgtctcaagcacaaaccccagccttagtttcgagactttgggagatagcggcagatatctagtttggcattttgtatat |
| | taattaagtctcgcaatggagcgctctgatgcggtgcagcgtcggctgcagcacctggcagtggcgctggggtcgccctatcgctcgg |
| | 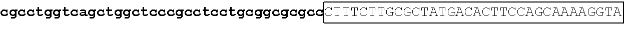 |
| | 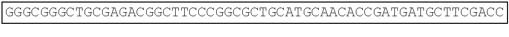 |
| | 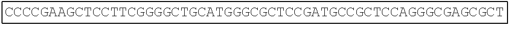 |
| | 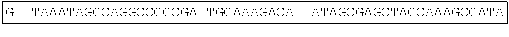 |
| | 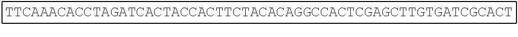 |
| | 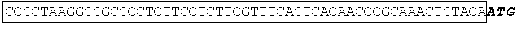 |
| | ctgctgcaggccttcctgttcctgctggccggcttcgccgccaagatcagcgcctccatgacgaacgagacgtccgaccgccccctggtg |
| | cacttcacccccaacaagggctggatgaacgaccccaacggcctgtggtacgacgagaaggacgccaagtggcacctgtacttccagt |
| | acaacccgaacgacaccgtctgggggacgcccttgttctggggccacgccacgtccgacgacctgaccaactgggaggaccagccca |
| | tcgccatcgccccgaagcgcaacgactccggcgccttctccggctccatggtggtggactacaacaacacctccggcttcttcaacgaca |
| | ccatcgacccgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctggtacgagc |
| | cctcccagaagtggatcatgaccgcggccaagtcccaggactacaagatcgagatctactcctccgacgacctgaagtcctggaagctg |
| | gagtccgcgttcgccaacgagggcttcctcggctaccagtacgagtgccccggcctgatcgaggtccccaccgagcaggaccccagca |
| | agtcctactgggtgatgttcatctccatcaaccccggcgccccggccggcggctccttcaaccagtacttcgtcggcagcttcaacggcac |
| | ccacttcgaggccttcgacaaccagtcccgcgtggtggacttcggcaaggactactacgccctgcagaccttcttcaacaccgacccgac |
| | ctacgggagcgccctgggcatcgcgtgggcctccaactgggagtactccgccttcgtgcccaccaacccctggcgctcctccatgtccctc |
| | gtgcgcaagttctccctcaacaccgagtaccaggccaacccggagacggagctgatcaacctgaaggccgagccgatcctgaacatc |
| | agcaacgccggcccctggagccggttcgccaccaacaccacgttgacgaaggccaacagctacaacgtcgacctgtccaacagcacc |
| | ggcaccctggagttcgagctggtgtacgccgtcaacaccacccagacgatctccaagtccgtgttcgcggacctctccctctggttcaagg |
| | gcctggaggaccccgaggagtacctccgcatgggcttcgaggtgtccgcgtcctccttcttcctggaccgcgggaacagcaaggtgaag |
| | ttcgtgaaggagaacccctacttcaccaaccgcatgagcgtgaacaaccagcccttcaagagcgagaacgacctgtcctactacaagg |
| | tgtacggcttgctggaccagaacatcctggagctgtacttcaacgacggcgacgtcgtgtccaccaacacctacttcatgaccaccggga |
| | acgccctgggctccgtgaacatgacgacgggggtggacaacctgttctacatcgacaagttccaggtgcgcgaggtcaagtgactTA |
| | AGACGCCCGCGCGGCGCACCTGACCTGTTCTCTCGAGGGCGCCTGTTCTGCCTTGCGA |
| | AACAAGCCCCTGGAGCATGCGTGCATGATCGTCTCTGGCGCCCCGCCGCGCGGTTTGT |
| | CGCCCTCGCGGGCGCCGCGGCCGCGGGGGCGCATTGAAATTGTTGCAAACCCCACCT |
| | GACAGATTGAGGGCCCAGGCAGGAAGGCGTTGAGATGGAGGTACAGGAGTCAAGTA |
| | ACTGAAAGTTTTTATGATAACTAACAACAAAGGGTCGTTTCTGGCCAGCGAATGACA |
| | AGAACAAGATTCCACATTTCCGTGTAGAGGCTTGCCATCGAATGTGAGCGGGCGGGC |
| | CGCGGACCCGACAAAACCCTTACGACGTGGTAAGAAAAACGTGGCGGGCACTGTCCC |
| | TGTAGCCTGAAGACCAGCAGGAGACGATCGGAAGCATCACAGCACAACCGGTgcagcag |
| | cagctcggatagtatcgacacactctggacgctggtcgtgtgatggactgttgccgccacacttgctgccttgacctgtgaatatccctgcc |
| | gcttttatcaaacagcctcagtgtgtttgatcttgtgtgtacgcgcttttgcgagttgctagctgcttgtgctatttgcgaataccacccccagca |
| | tccccttccctcgtttcatatcgcttgcatcccaaccgcaacttatctacgctgtcctgctatccctcagcgctgctcctgctcctgctcactgcc |
| | cctcgcacagccttggtttgggctccgcctgtattctcctggtactgcaacctgtaaaccagcactgcaatgctgatgcacgggaagtagtg |
| | 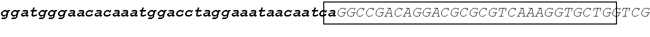 |
| | 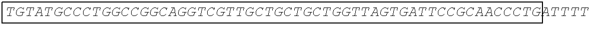 |
| | 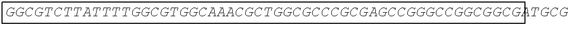 |
| | 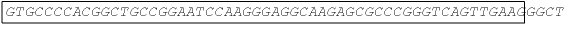 |
| | 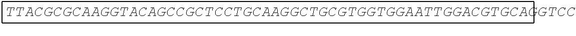 |
| | 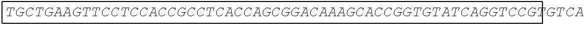 |
| | 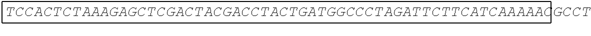 |
| | 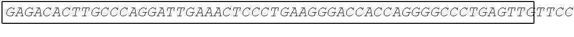 |
| | 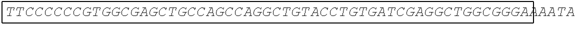 |
| | 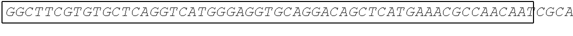 |
| | 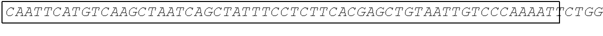 |
| | 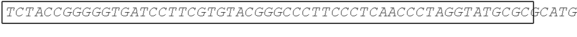 |
| | 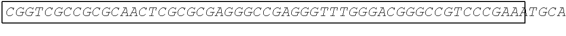 |
| | 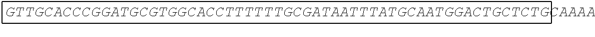 |
| | 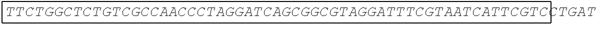 |
| | 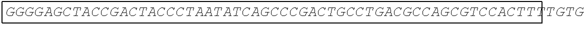 |
| | 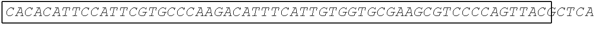 |
| | 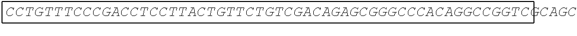 |
| | 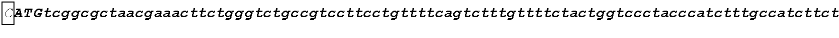 |
| | accgtatccgcttcgcctcgctgggcaagcgcaacgacatgctggactgggcccgctccctcgtggggttctttcgggtgacgcttttgcaggcag |
| | gcgaacagggtctgtacaagggaggcccttgcctatacctgtgcaaccaccgctcttgggccgacttctttgtcgacgcctacctaacgga |
| | gggtcgcgctgccctgatgagccgctggcttgtgtacttcgtgttcccggtgttctgtacctcgtgcatgatcctgaagggtatagtcctgttta |
| | agcgcgggaccatcgcggacaaggaggcgtttaacgcctggctggaccggaccctgggtgactctcacgtccctggcctcctggtctac |
| | ccggagggacaccggagcaccaagcctacctcgctgcccttgaagcgaggcatgctgcactacgcgcattcccggaagctgccagtcc |
| | aggtcatcgtcacgcgcggcaaagacgaggtcatgtccgagaaaggccagtccgttcacttcggccgcacctgcgtcacgaccttctcc |
| | aaggtcctcaagccagccgactacccgaactttgaggcctttttcgcgacctccaggctacctgggacgcctgctgggcagccacctac |
| | ggcctgaaggacctgaagggcgtgccgcgcttctcgatgccctctccccaggcttacacgtacagcgccaagacctgggcacagcaac |
| | tggcgatcacgctcgtgtcgatcgtggtttttgcggccgtctgttacggctcctggcgtggccttgccgcagccctcgctgccacgggtgctg |
| | cgcaacaggtccgcggccagcaacagcaacagcaacagcaacagcgccgacggcgcctcagctcctctgggggatgggccgaggg |
| | tcgcggcgcccaggccgggggacgcctccagtgggacgggggcgactgggggcggtgggtggcgctgcccctgtccctggaggtggt |
| | cacggtccttctggctgcgtgggtcggaggcagcgtgctcaggtccttcctgTAGttaattaagcagcagcagctcggatagtatcgacacact |
| | ctggacgctggtcgtgtgatggactgttgccgccacacttgctgccttgacctgtgaatatccctgccgcttttatcaaacagcctcagtgtgtttg |
| | atcttgtgtgtacgcgcttttgcgagttgctagctgcttgtgctatttgcgaataccacccccagcatccccttccctcgtttcatatcgcttgcat |
| | cccaaccgcaacttatctacgctgtcctgctatccctcagcgctgctcctgctcctgctcactgcccctcgcacagccttggtttgggctccgcct |
| | gtattctcctggtactgcaacctgtaaaccagcactgcaatgctgatgcacgggaagtagtgggatgggaacacaaatggaagcgtctgcgt |
| | gttgggagctggagtcgtgggcttgacgacggcgctgcagctcttgcaggatgtgcctggcgtgcgcgttcacgtcgtggctgagaaa |
| | tatggcgacgaaacgttgacggctggggccggcgggctgtggatgccatacgcattgggtacgcggccattggatgggattggtagg |
| | cttatggagggataatagagtttttgtcggatccaacgcatgttgatgcagtagcccggtgtgctgaaagtatggaaggatggtgcatt |
| | ggctattcacatgcgctgcccaccccttttcgcaggaaatgtgccggcatcgttggtgcaccgatggggaaaatcgacgttcgaccac |
| | tacatgaaaatgtatacctctgaagatgcagcaactgcgggtgcgaaacggataacggtttggtaggtgtatgtcgcagcatgtgctg |
| | gattttgcgggcaactccccctgccacggcccattgcaggtgtcatgttgactggaggatacgaactttcggccgtcaaattcccagag |
| | gaggaccccctctgggccgacattgtgcccactgctcttccgcttgctgtttcctgtgtgaaattgt |

The construct can be written as 5′DAO1b::CrTUB2:ScSUC2:PmPGH:CvNR:PmAMT03:ChsLPAAT2:CvNR::3′DAO1b. Proceeding in the 5′ to 3′ direction, bold, lowercase sequences represent genomic DNA from CHK22 that permit targeted integration at the DAO1b locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text followed by a linker. The P. moriformis PmAMT03 promoter, indicated by boxed italicized text, drives the expression of the ChsLPAAT2. The initiator ATG and terminator TGA codons of the ChsLPAAT2 are indicated by uppercase, bold italics, while the remainder of the gene is indicated by bold italics. The C. vulgaris nitrate reductase 3′-UTR is indicated by lowercase underlined text followed by the CHK22 DAO1b genomic region indicated by bold, lowercase text.

Table 19 shows the fatty acid/TAG profiles of six primary transformants of CHK22 transformed with pCHK786. The P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 19
Screen of primary transformants of CHK22 with pCHK786.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK786- | 0.06 | 1.64 | 22.11 | 3.43 | 57.43 | 12.61 | 0.55 | 0.32 | 0.26 | 1.56 |
| 1 | | | | | | | | | | |
| pCHK786- | 0.06 | 1.63 | 21.58 | 3.52 | 56.71 | 14.04 | 0.55 | 0.04 | 0.27 | 1.99 |
| 2 | | | | | | | | | | |
| pCHK786- | 0.06 | 1.80 | 26.04 | 2.82 | 55.77 | 9.53 | 0.57 | 0.07 | 1.61 | 1.50 |
| 3 | | | | | | | | | | |
| pCHK786- | 0.04 | 1.48 | 25.14 | 2.93 | 58.91 | 9.00 | 0.49 | 0.25 | 0.25 | 1.58 |
| 4 | | | | | | | | | | |
| pCHK786- | 0.04 | 1.47 | 24.92 | 3.10 | 59.00 | 8.98 | 0.48 | 0.29 | 0.27 | 1.50 |
| 5 | | | | | | | | | | |
| pCHK786- | 0.06 | 1.60 | 21.73 | 3.35 | 57.88 | 12.90 | 0.55 | 0.04 | 0.31 | 1.60 |
| 6 | | | | | | | | | | |
| CHK22 | 0.04 | 1.52 | 26.43 | 2.97 | 58.76 | 7.88 | 0.44 | 0.25 | 0.23 | 1.53 |
| Control | | | | | | | | | | |

### Example 11: Expressing Chlamydomonas incerta LPAAT2 (CiLPAAT2) in P. moriformis Strain CHK22

C. incerta CiLPAAT2 was introduced into a P. moriformis strain CHK22. The expression construct pCHK787 contained 5Y and 3Y homology arms to permit targeted integration of CiLPAAT2 into the genome and is shown in Table 20.

TABLE 20
Integrative sequences for the transformation of P. moriformis with pCHK787
encoding CiLPAAT2.

| SEQ | |
|---|---|
| ID | |
| NO: | Sequence |
| 10 | gctcttccgcttgcccgcaccctcgttgatctgggagccctgcgcagccccttaaatcatctcagtcaggtttctatgttcaactgagcct |
| | aaagggctttcgtcacgcgcacgagcacacgtatatcggctacgcagtctctcagaagcggtagaacagttcgcaagccctcgtcgg |
| | tcgaaaacttgcgccagtactattgaattaaattaaatgatcgaatgagacgcgaaacttttgcagaatgccactgagtttgcccaga |
| | gaatgggagtggcgccattcaccatccgcctgtgcacggcctgattcgccgagacgatgaatggcgagaccagggagcggcttgcg |
| | agccccgagccggtagcaggaataacggtcgacaatcttcctgtccaattactggcaaccattagaaagagccggagcgcgttgaa |
| | attctgcaatcgagtaatttttcgatgcggcgggcctgctgaaccctaaggctccggcctatgtttaaggcgatccaagatgcacgcgg |
| | ccccaggcacgtgtctcaagcacaaaccccagccttagtttcgagactttgggagatagcggcagatatctagtttggcattttgtatat |
| | taattaagtctcgcaatggagcgctctgatgcggtgcagcgtcggctgcagcacctggcagtggcgctggggtcgccctatcgctcgg |
| | 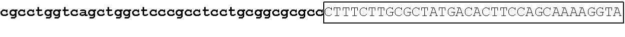 |
| | 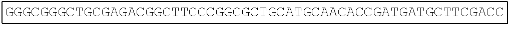 |
| | 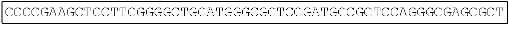 |
| | 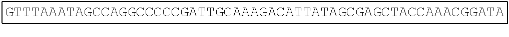 |
| | 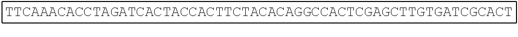 |
| | 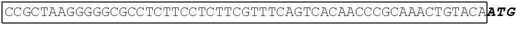 |
| | ctgctgcaggccttcctgttcctgctggccggcttcgccgccaagatcagcgcctccatgacgaacgagacgtccgaccgccccctggtg |
| | cacttcacccccaacaagggctggatgaacgaccccaacggcctgtggtacgacgagaaggacgccaagtggcacctgtacttccagt |
| | acaacccgaacgacaccgtctgggggacgcccttgttctggggccacgccacgtccgacgacctgaccaactgggaggaccagccca |
| | tcgccatcgccccgaagcgcaacgactccggcgccttctccggctccatggtggtggactacaacaacacctccggcttcttcaacgaca |
| | ccatcgacccgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctggtacgagc |
| | cctcccagaagtggatcatgaccgcggccaagtcccaggactacaagatcgagatctactcctccgacgacctgaagtcctggaagctg |
| | gagtccgcgttcgccaacgagggcttcctcggctaccagtacgagtgccccggcctgatcgaggtccccaccgagcaggaccccagca |
| | agtcctactgggtgatgttcatctccatcaaccccggcgccccggccggcggctccttcaaccagtacttcgtcggcagcttcaacggcac |
| | ccacttcgaggccttcgacaaccagtcccgcgtggtggacttcggcaaggactactacgccctgcagaccttcttcaacaccgacccgac |
| | ctacgggagcgccctgggcatcgcgtgggcctccaactgggagtactccgccttcgtgcccaccaacccctggcgctcctccatgtccctc |
| | gtgcgcaagttctccctcaacaccgagtaccaggccaacccggagacggagctgatcaacctgaaggccgagccgatcctgaacatc |
| | agcaacgccggcccctggagccggttcgccaccaacaccacgttgacgaaggccaacagctacaacgtcgacctgtccaacagcacc |
| | ggcaccctggagttcgagctggtgtacgccgtcaacaccacccagacgatctccaagtccgtgttcgcggacctctccctctggttcaagg |
| | gcctggaggaccccgaggagtacctccgcatgggcttcgaggtgtccgcgtcctccttcttcctggaccgcgggaacagcaaggtgaag |
| | ttcgtgaaggagaacccctacttcaccaaccgcatgagcgtgaacaaccagcccttcaagagcgagaacgacctgtcctactacaagg |
| | tgtacggcttgctggaccagaacatcctggagctgtacttcaacgacggcgacgtgtgtccaccaacacctacttcatgaccaccggga |
| | acgccctgggctccgtgaacatgacgacgggggtggacaacctgttctacatcgacaagttccaggtgcgcgaggtcaagtgactTA |
| | AGACGCCCGCGCGGCGCACCTGACCTGTTCTCTCGAGGGCGCCTGTTCTGCCTTGCGA |
| | AACAAGCCCCTGGAGCATGCGTGCATGATCGTCTCTGGCGCCCCGCCGCGCGGTTTGT |
| | CGCCCTCGCGGGCGCCGCGGCCGCGGGGGCGCATTGAAATTGTTGCAAACCCCACCT |
| | GACAGATTGAGGGCCCAGGCAGGAAGGCGTTGAGATGGAGGTACAGGAGTCAAGTA |
| | ACTGAAAGTTTTTATGATAACTAACAACAAAGGGTCGTTTCTGGCCAGCGAATGACA |
| | AGAACAAGATTCCACATTTCCGTGTAGAGGCTTGCCATCGAATGTGAGCGGGCGGGC |
| | CGCGGACCCGACAAAACCCTTACGACGTGGTAAGAAAAACGTGGCGGGCACTGTCCC |
| | TGTAGCCTGAAGACCAGCAGGAGACGATCGGAAGCATCACAGCACAACCGGTgcagcag |
| | cagctcggatagtatcgacacactctggacgctggtcgtgtgatggactgttgccgccacacttgctgccttgacctgtgaatatccctgccgc |
| | ttttatcaaacagcctcagtgtgtttgatcttgtgtgtacgcgcttttgcgagttgctagctgcttgtgctatttgcgaataccacccccagca |
| | tccccttccctcgtttcatatcgcttgcatcccaaccgcaacttatctacgctgtcctgctatccctcagcgctgctcctgctcctgctcactg |
| | cccctcgcacagccttggtttgggctccgcctgtattctcctggtactgcaacctgtaaaccagcactgcaatgctgatgcacgggaagtagtg |
| | 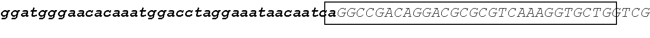 |
| | 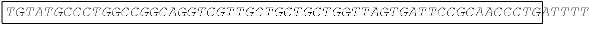 |
| | 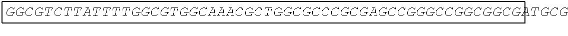 |
| | 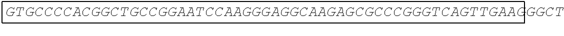 |
| | 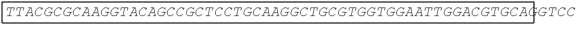 |
| | 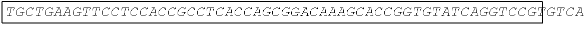 |
| | 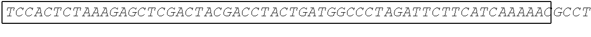 |
| | 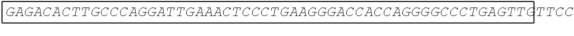 |
| | 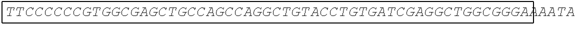 |
| | 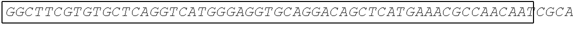 |
| | 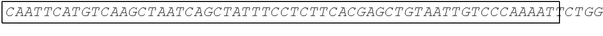 |
| | 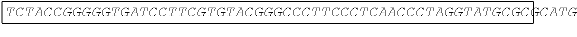 |
| | 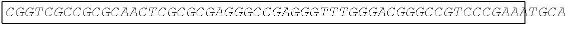 |
| | 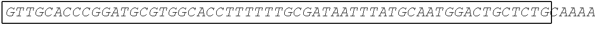 |
| | 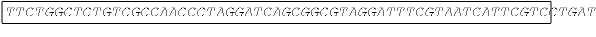 |
| | 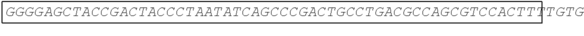 |
| | 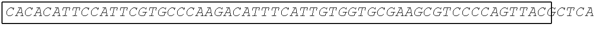 |
| | 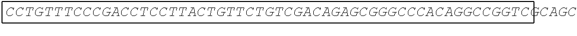 |
| | 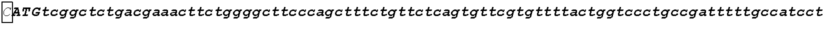 |
| | ataccgcattcgctttgcgagcctgggcaagcgcaacgacatgctggactgggcgcgcgcgttagtcgggtttttccgcgtgacgctgttaca |
| | ggctggcgagcagagcctgtacaaagggggtccgtgcctctacctgtgcaaccatcggtcgtgggctgacttctttatcgacgcctacctgacg |
| | gagggccgggccgcgctgatgtcgcggtggctcgtgtactttgtctttcctgtgttctgcacctcctgcatggtcttgaagggcatcgtcct |
| | gttcaagcgcggtacaatcgcggacaaggaggccttcaacgcttggctcgaccggaccctcggcgacagccatgtgcccggcctcctggtc |
| | tacccggagggacaccgctccaccaagcctgcgtcactgccgctcaagaggggcatgctgcactacgctcactcccgcaagctcccgg |
| | tgcaggtcattatcacacggggtaaggacgaggtcctgagtgagaagtcccaatcggtgcacttcggccgcacctgcgtgacgacctttt |
| | cgaaggtgatcaagccctcggacttccccaacttcgaggcgttctttgccgatctccaggcaacctgggacgcgggctggacggccacct |
| | acggcctgaaggacctcaagggcgtcccccgattctccatgcccagcccgcaggcgtacacgtactcaagcaagatgtgggcgcagc |
| | aactggcgatcacccttttcagcattccggtctttgcaggcgtctgctacggtagctggcgcgtcttggctgccgcgctggctgccacgggtg |
| | ccgctcaacaggtggtcaccctcgccttagctggctgggtgggaggctcggtgctgcggagctttctgTAGttaattaagcagcagcagctcgg |
| | atagtatcgacacactctggacgctggtcgtgtgatggactgttgccgccacacttgctgccttgacctgtgaatatccctgccgcttttatc |
| | aaacagcctcagtgtgtttgatcttgtgtgtacgcgcttttgcgagttgctagctgcttgtgctatttgcgaataccacccccagcatccccttc |
| | cctcgtttcatatcgcttgcatcccaaccgcaacttatctacgctgtcctgctatccctcagcgctgctcctgctcctgctcactgcccctcgca |
| | cagccttggtttgggctccgcctgtattctcctggtactgcaacctgtaaaccagcactgcaatgctgatgcacgggaagtagtgggatgggaa |
| | cacaaatggaagcgtctgcgtgttgggagctggagtcgtgggcttgacgacggcgctgcagctcttgcaggatgtgcctggcgtgcgcgttca |
| | cgtcgtggctgagaaatatggcgacgaaacgttgacggctggggccggcgggctgtggatgccatacgcattgggtacgcggccatt |
| | ggatgggattggtaggcttatggagggataatagagtttttgtcggatccaacgcatgttgatgcagtagcccggtgtgctgaaagtat |
| | ggaaggatggtgcattggctattcacatgcgctgcccaccccttttcgcaggaaatgtgccggcatcgttggtgcaccgatggggaaa |
| | atcgacgttcgaccactacatgaaaatgtatacctctgaagatgcagcaactgcgggtgcgaaacggataacggtttggtaggtgtat |
| | gtcgcagcatgtgctggattttgcgggcaactccccctgccacggcccattgcaggtgtcatgttgactggaggatacgaactttcggc |
| | cgtcaaattcccagaggaggaccccctctgggccgacattgtgcccactgctcttccgcttgctgtttcctgtgtgaaattgt |

The construct can be written as 5′ DAO1b::CrTUB2:ScSUC2:PmPGH:CvNR:PmAMT03:CiLPAAT2:CvNR::3′DAO1b. Proceeding in the 5Y to 3Y direction, bold, lowercase sequences represent genomic DNA from CHK22 that permit targeted integration at the DAO1b locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text followed by a linker, indicated by lowercase, bold italics. The P. moriformis PmAMT03 promoter, indicated by boxed italicized text, drives the expression of the CiLPAAT2. The initiator ATG and terminator TGA codons of the CiLPAAT2 are indicated by uppercase, bold italics, while the remainder of the gene is indicated by bold italics. The C. vulgaris nitrate reductase 3′-UTR is indicated by lowercase underlined text followed by the CHK22 DAO1b genomic region indicated by bold, lowercase text.

Table 21 shows the fatty acid/TAG profiles of seven primary transformants of CHK22 transformed with pCHK787. P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 21
Screen of primary transformants of CHK22 with pCHK787.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK787- | 0.06 | 1.59 | 21.37 | 3.80 | 56.59 | 13.98 | 0.55 | 0.27 | 0.27 | 2.13 |
| 1 | | | | | | | | | | |
| pCHK787- | 0.05 | 1.55 | 21.51 | 3.25 | 57.93 | 12.91 | 0.61 | 0.30 | 0.28 | 1.55 |
| 2 | | | | | | | | | | |
| pCHK787- | 0.06 | 1.56 | 22.21 | 3.60 | 57.26 | 12.69 | 0.52 | 0.27 | 0.28 | 1.95 |
| 3 | | | | | | | | | | |
| pCHK787- | 0.04 | 1.46 | 25.58 | 3.15 | 58.81 | 8.53 | 0.46 | 0.26 | 0.25 | 1.59 |
| 4 | | | | | | | | | | |
| pCHK787- | 0.06 | 1.54 | 21.36 | 3.69 | 57.17 | 13.54 | 0.53 | 0.29 | 0.30 | 2.21 |
| 5 | | | | | | | | | | |
| pCHK787- | 0.06 | 1.55 | 22.40 | 3.84 | 57.60 | 12.16 | 0.51 | 0.03 | 0.29 | 2.04 |
| 6 | | | | | | | | | | |
| pCHK787 | 0.05 | 1.65 | 23.19 | 3.06 | 57.86 | 11.70 | 0.56 | 0.04 | 0.26 | 1.53 |
| CHK22 | 0.04 | 1.52 | 26.43 | 2.97 | 58.76 | 7.88 | 0.44 | 0.25 | 0.23 | 1.53 |
| Control | | | | | | | | | | |

### Example 12: Expressing Volvox africanus LPAAT2 (VaLPAAT2) in P. moriformis Strain CHK22

V. africanus VaLPAAT2 was introduced into a P. moriformis strain CHK22. The expression construct pCHK788 contained 5′ and 3′ homology arms to permit targeted integration of VaLPAAT2 into the genome and is shown in Table 22.

TABLE 22
Integrative sequences for the transformation of P. moriformis with pCHK788
encoding VaLPAAT2.

| SEQ | |
|---|---|
| ID | |
| NO: | Sequence |
| 11 | gctcttccgcttgcccgcaccctcgttgatctgggagccctgcgcagccccttaaatcatctcagtcaggtttctatgttcaactgagcct |
| | aaagggctttcgtcacgcgcacgagcacacgtatatcggctacgcagtctctcagaagcggtagaacagttcgcaagccctcgtcgg |
| | tcgaaaacttgcgccagtactattgaattaaattaaatgatcgaatgagacgcgaaacttttgcagaatgccactgagtttgcccaga |
| | gaatgggagtggcgccattcaccatccgcctgtgcacggcctgattcgccgagacgatgaatggcgagaccagggagcggcttgcg |
| | agccccgagccggtagcaggaataacggtcgacaatcttcctgtccaattactggcaaccattagaaagagccggagcgcgttgaa |
| | attctgcaatcgagtaatttttcgatgcgggggcctgctgaaccctaaggctccggcctatgtttaaggcgatccaagatgcacgcgg |
| | ccccaggcacgtgtctcaagcacaaaccccagccttagtttcgagactttgggagatagcggcagatatctagtttggcattttgtatat |
| | taattaagtctcgcaatggagcgctctgatgcggtgcagcgtcggctgcagcacctggcagtggcgctggggtcgccctatcgctcgg |
| | 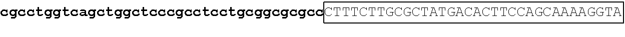 |
| | 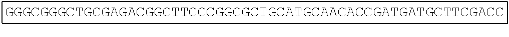 |
| | 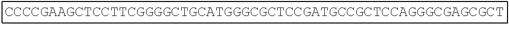 |
| | 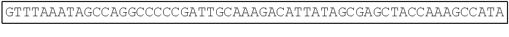 |
| | 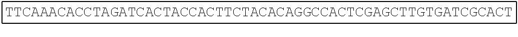 |
| | 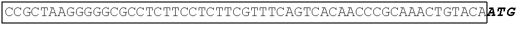 |
| | ctgctgcaggccttcctgttcctgctggccggcttcgccgccaagatcagcgcctccatgacgaacgagacgtccgaccgccccctggtg |
| | cacttcacccccaacaagggctggatgaacgaccccaacggcctgtggtacgacgagaaggacgccaagtggcacctgtacttccagt |
| | acaacccgaacgacaccgtctgggggacgcccttgttctggggccacgccacgtccgacgacctgaccaactgggaggaccagccca |
| | tcgccatcgccccgaagcgcaacgactccggcgccttctccggctccatggtggtggactacaacaacacctccggcttcttcaacgaca |
| | ccatcgacccgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctggtacgagc |
| | cctcccagaagtggatcatgaccgcggccaagtcccaggactacaagatcgagatctactcctccgacgacctgaagtcctggaagctg |
| | gagtccgcgttcgccaacgagggcttcctcggctaccagtacgagtgccccggcctgatcgaggtccccaccgagcaggaccccagca |
| | agtcctactgggtgatgttcatctccatcaaccccggcgccccggccggcggctccttcaaccagtacttcgtcggcagcttcaacggcac |
| | ccacttcgaggccttcgacaaccagtcccgcgtggtggacttcggcaaggactactacgccctgcagaccttcttcaacaccgacccgac |
| | ctacgggagcgccctgggcatcgcgtgggcctccaactgggagtactccgccttcgtgcccaccaacccctggcgctcctccatgtccctc |
| | gtgcgcaagttctccctcaacaccgagtaccaggccaacccggagacggagctgatcaacctgaaggccgagccgatcctgaacatc |
| | agcaacgccggcccctggagccggttcgccaccaacaccacgttgacgaaggccaacagctacaacgtcgacctgtccaacagcacc |
| | ggcaccctggagttcgagctggtgtacgccgtcaacaccacccagacgatctccaagtccgtgttcgcggacctctccctctggttcaagg |
| | gcctggaggaccccgaggagtacctccgcatgggcttcgaggtgtccgcgtcctccttcttcctggaccgcgggaacagcaaggtgaag |
| | ttcgtgaaggagaacccctacttcaccaaccgcatgagcgtgaacaaccagcccttcaagagcgagaacgacctgtcctactacaagg |
| | tgtacggcttgctggaccagaacatcctggagctgtacttcaacgacggcgacgtcgtgtccaccaacacctacttcatgaccaccggga |
| | acgccctgggctccgtgaacatgacgacgggggtggacaacctgttctacatcgacaagttccaggtgcgcgaggtcaagtgactTA |
| | AGACGCCCGCGCGGCGCACCTGACCTGTTCTCTCGAGGGCGCCTGTTCTGCCTTGCGA |
| | AACAAGCCCCTGGAGCATGCGTGCATGATCGTCTCTGGCGCCCCGCCGCGCGGTTTGT |
| | CGCCCTCGCGGGCGCCGCGGCCGCGGGGGCGCATTGAAATTGTTGCAAACCCCACCT |
| | GACAGATTGAGGGCCCAGGCAGGAAGGCGTTGAGATGGAGGTACAGGAGTCAAGTA |
| | ACTGAAAGTTTTTATGATAACTAACAACAAAGGGTCGTTTCTGGCCAGCGAATGACA |
| | AGAACAAGATTCCACATTTCCGTGTAGAGGCTTGCCATCGAATGTGAGCGGGCGGGC |
| | CGCGGACCCGACAAAACCCTTACGACGTGGTAAGAAAAACGTGGCGGGCACTGTCCC |
| | TGTAGCCTGAAGACCAGCAGGAGACGATCGGAAGCATCACAGCACAACCGGTgcagcag |
| | cagctcggatagtatcgacacactctggacgctggtcgtgtgatggactgttgccgccacacttgctgccttgacctgtgaatatccctgcc |
| | gcttttatcaaacagcctcagtgtgtttgatcttgtgtgtacgcgcttttgcgagttgctagctgcttgtgctatttgcgaataccacccccagca |
| | tccccttccctcgtttcatatcgcttgcatcccaaccgcaacttatctacgctgtcctgctatccctcagcgctgctcctgctcctgctcactgcc |
| | cctcgcacagccttggtttgggctccgcctgtattctcctggtactgcaacctgtaaaccagcactgcaatgctgatgcacgggaagtagtg |
| | 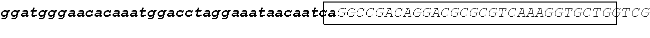 |
| | 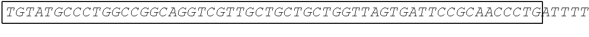 |
| | 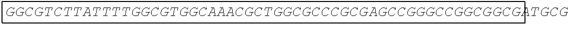 |
| | 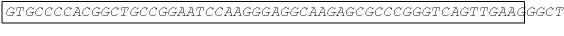 |
| | 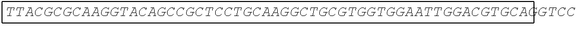 |
| | 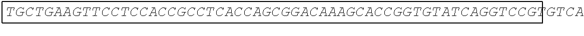 |
| | 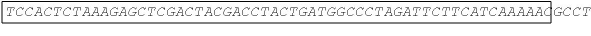 |
| | 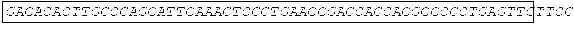 |
| | 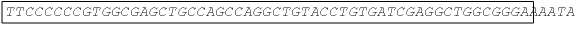 |
| | 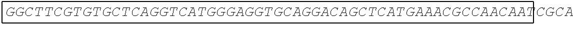 |
| | 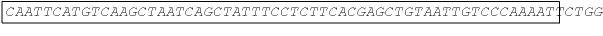 |
| | 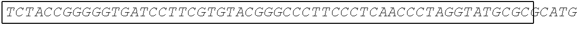 |
| | 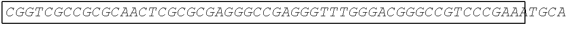 |
| | 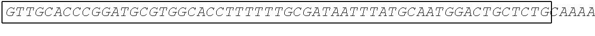 |
| | 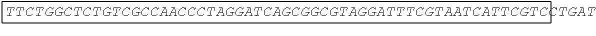 |
| | 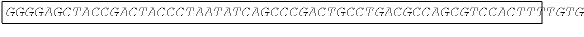 |
| | 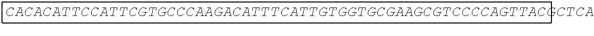 |
| | 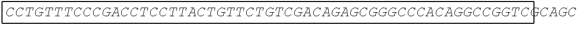 |
| | 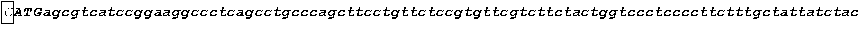 |
| | cgtatccgcttcatcaacgtcggcaagcgcaacgacatgctggactgggcgaaggcgctggtcgcgtttttccatgtcaccgtcctcaagc |
| | agggcgagatgacgctgtaccgcggtggccgctgtctgtacctgtgcaaccaccgctcctgggccgactttttcatcgacacctacctgac |
| | cgagggtcgtgccgcgctcatgtcccgctggctggtgtactttgtgttccccgtcttttgcagctcggtaatcgtcctccgtggcatcgtcctgtt |
| | caagcgcggccacattgcgaataaggaggcgtttaacgcatggctggatgtgacgctcggcaagtcgacagtccccggcctcctggtgt |
| | acccagagggtcaccgtagcctgaagcccacctccctgcccctgaaacgcggcatgctgcacttcgcctactcccggaagctcccggtc |
| | caaattatcgtgacgcgtggcaaggaagaggtgctctccgagaagactctgtcggcgcgctggggcaggacactggtggtcaccttctc |
| | gcacgtcctcaagccgtccgactacgattcgttcgatgcgtttttcgatgacatccagcataagtgggacgcttgctggaagtccagctatg |
| | acaagcccacgggggcgtgccagaactctccatgaagggggcgcacgagtacgactacccccgcagcatgtggtggctgcaactgg |
| | gcatgaccctaatgtcgatcgtcatcttgatcggggtgatgtgggggtcctgggtcgcgctgcgcacgggcctggccgtcctcggggccgc |
| | acagcaacccgtggttctgtgcgccgcggtctggatcggcgcctccatgctccgcgccttcttcTAGttaattaagcagcagcagctcggatagta |
| | tcgacacactctggacgctggtcgtgtgatggactgttgccgccacacttgctgccttgacctgtgaatatccctgccgcttttatcaaacagcct |
| | cagtgtgtttgatcttgtgtgtacgcgcttttgcgagttgctagctgcttgtgctatttgcgaataccacccccagcatccccttccctcgttt |
| | catatcgcttgcatcccaaccgcaacttatctacgctgtcctgctatccctcagcgctgctcctgctcctgctcactgcccctcgcacagccttgg |
| | tttgggctccgcctgtattctcctggtactgcaacctgtaaaccagcactgcaatgctgatgcacgggaagtagtgggatgggaacacaaatgg |
| | aagcgtctgcgtgttgggagctggagtcgtgggcttgacgacggcgctgcagctcttgcaggatgtgcctggcgtgcgcgttcacgtc |
| | gtggctgagaaatatggcgacgaaacgttgacggctggggccggcgggctgtggatgccatacgcattgggtacgcggccattgga |
| | tgggattggtaggcttatggagggataatagagtttttgtcggatccaacgcatgttgatgcagtagcccggtgtgctgaaagtatgga |
| | aggatggtgcattggctattcacatgcgctgcccaccccttttcgcaggaaatgtgccggcatcgttggtgcaccgatggggaaaatc |
| | gacgttcgaccactacatgaaaatgtatacctctgaagatgcagcaactgcgggtgcgaaacggataacggtttggtaggtgtatgtc |
| | gcagcatgtgctggattttgcgggcaactccccctgccacggcccattgcaggtgtcatgttgactggaggatacgaactttcggccgt |
| | caaattcccagaggaggaccccctctgggccgacattgtgcccactgctcttccgcttgctgtttcctgtgtgaaattgt |

The construct can be written as 5′DAO1b::CrTUB2:ScSUC2:PmPGH:CvNR:PmAMT03:VaLPAAT2:CvNR::3′DAO1b. Proceeding in the 5′ to 3′ direction, bold, lowercase sequences represent genomic DNA from CHK22 that permit targeted integration at the DAO1b locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text followed by a linker, indicated by lowercase, bold italics. The P. moriformis PmAMT03 promoter, indicated by boxed italicized text, drives the expression of the VaLPAAT2. The initiator ATG and terminator TGA codons of the VaLPAAT2 are indicated by uppercase, bold italics, while the remainder of the gene is indicated by bold italics. The C. vulgaris nitrate reductase 3′-UTR is indicated by lowercase underlined text followed by the CHK22 DAO1b genomic region indicated by bold, lowercase text.

Table 23 shows the fatty acid/TAG profiles of six primary transformants of CHK22 transformed with pCHK788. P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMES for subsequent quantitation and characterization by GC/FID. TAG analysis was conducted using the LCMS method as described above.

TABLE 23
Screen of primary transformants of CHK22 with pCHK788.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK788- | 0.05 | 1.60 | 22.85 | 3.53 | 57.65 | 11.64 | 0.50 | 0.29 | 0.28 | 1.76 |
| 1 | | | | | | | | | | |
| pCHK788- | 0.05 | 1.55 | 21.80 | 3.70 | 57.40 | 12.85 | 0.53 | 0.29 | 0.28 | 1.76 |
| 2 | | | | | | | | | | |
| pCHK788- | 0.05 | 1.61 | 23.72 | 3.19 | 57.12 | 11.72 | 0.54 | 0.04 | 0.39 | 1.96 |
| 3 | | | | | | | | | | |
| pCHK788- | 0.06 | 1.62 | 21.68 | 3.88 | 56.61 | 13.71 | 0.55 | 0.03 | 0.31 | 1.96 |
| 4 | | | | | | | | | | |
| pCHK788- | 0.05 | 1.58 | 24.35 | 3.27 | 57.83 | 10.37 | 0.47 | 0.29 | 0.27 | 1.68 |
| 5 | | | | | | | | | | |
| pCHK788- | 0.06 | 1.55 | 21.86 | 3.61 | 57.60 | 12.88 | 0.52 | 0.04 | 0.31 | 1.86 |
| 6 | | | | | | | | | | |
| CHK22 | 0.04 | 1.52 | 26.43 | 2.97 | 58.76 | 7.88 | 0.44 | 0.25 | 0.23 | 1.53 |
| Control | | | | | | | | | | |

As the transformants of CHK22 expressing CrLPAAT2 produced the higher OP:00 ratio, the percent identity (%) of all the amino acid sequences across the entire LPAAT protein was compared against that of CrLPAAT2. The results are shown in Table 24. The resulted phenotype of expressing these LPAATs in CHK22 is indicated with the m/z (577.5/603.5) OP:OO ratio; the OP:OO ratio shown in the rows of PmLPAAT1 or PmLPAAT2 represents the analytical results of the non-transgenic line CHK22.

In some embodiments, a microalgal cell provided herein comprises an exogenous gene that encodes for a LPAAT in Table 24. In some embodiments, the exogenous gene can comprise a sequence with at least 50%, at least 60%, at least 70%, at least 80%, at least 90%, at least 95%, at least 96%, at least 97%, at least 98%, at least 99%, or 100% sequence identity to a LPAAT in Table 24.

TABLE 24
OP:OO Ratio

| | | | Identity | m/z (577.5/603.5) |
|---|---|---|---|---|
| Gene Name | Organism | GenBank ID | (%) | OP:OO |
| CrLPAAT2 | Chlamydomonas | XP_001699586.2 | 100.0 | 3.45 |
| | reinhardtii | | | |
| CiLPAAT2 | Chlamydomonas | KAG2426747.1 | 89.8 | 2.21 |
| | incerta | | | |
| ChsLPAAT2 | Chlamydomonas | KAG2454274.1 | 77.2 | 1.99 |
| | schloesseri | | | |
| VaLPAAT2 | Volvox africanus | GIL63906.1 | 69.2 | 1.96 |
| NoLPAT3 | Nannochloropsis | BAW99691.1 | 7.6 | 1.6 |
| | oceanica | | | |
| Sll1848 | Synechocystis sp | WP_010873224.1 | 17.7 | 1.60 |
| CrLPAAT1 | Chlamydomonas | XP_042921325.1 | 15.5 | 1.59 |
| | reinhardtii | | | |
| EcPlsC | Escherichia coli | HBN6599230.1 | 10.5 | 1.59 |
| VcLPAAT2_98847_V1 | Volvox | XP_002957774.1 | 47.5 | 1.58 |
| | carteri_98847 | | | |
| BnBAT2 | Brasscia napus | NP_001302928.1 | 14.1 | 1.57 |
| NoLPAT4 | Nannochloropsis | BAW99690.1 | 11.2 | 1.55 |
| | oceanica | | | |
| AtLPAAT2_ABG48392 | Arabidopsis | NP_567052.1 | 11.7 | |
| | thaliana_{—} | | | |
| | ABG48392 | | | |
| BnBAT1.13 | Brasscia | NP_001302981.1 | 11.7 | |
| | napus_ADC97478 | | | |
| BnBAT1.5 | Brasscia | NP_001302955.1 | 10.5 | |
| | napus_ADC97479 | | | |
| CnLPAAT2 | Cocos nucifera | Q42670.1 | 14.4 | |
| CosLPAAT2 | Coccomyxa | XP_005647638.1 | 47.2 | |
| | subellipsoidea | | | |
| CuPSR23_LPAAT2-1 | Cuphea PRS23 | LG018472.1 | 12.2 | |
| CuPSR23_LPAAT3-1 | Cuphea PRS23 | LG018473.1 | 13.3 | |
| CuPSR23_LPAATx | Cuphea PSR23 | LG018471.1 | 16.2 | |
| LaLPAAT2 | Limnanthes | Q42868.1 | 13.2 | |
| | alba_AAC49185 | | | |
| MaLPAAT2 | Mortierella | XP_002501997.1 | 9.383 | |
| | alpina_AED33305 | | | |
| MpLPAAT2 | Micrommia | XP_002501997.1 | 9.4 | |
| | pusilla_XP_{—} | | | |
| | 002501997 | | | |
| OlLPAAT2 | Ostreococcus | XP_001421357.1 | 32.8 | |
| | lucimarinus | | | |
| ScSLC1 | Saccharomyces | NP_010231.1 | 13.2 | |
| | cerevisiae | | | |
| PmLPAAT1 | Prototheca | N/A | 8.4 | 1.54 |
| | moriformis | | | |
| PmLPAAT2 | Prototheca | N/A | 32.1 | 1.54 |
| | moriformis | | | |

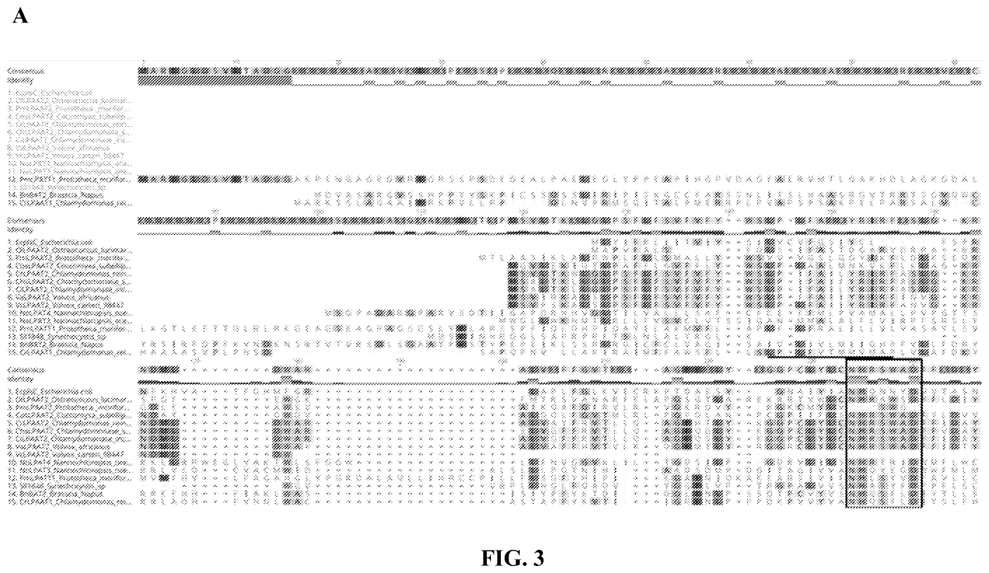

Table 25 shows the amino acid sequences of LPAATs described in the Examples herein. FIG. 3, Panels A-C illustrate amino acid sequence alignments of the amino acid sequences of LPAATs described herein, including EcPlsC (SEQ ID NO: 22), OlLPAAT2 (SEQ ID NO: 63), PmLPAAT2 (SEQ ID NO: 24), CosLPAAT2 (SEQ ID NO: 64), CrLPAAT2 (SEQ ID NO: 13), ChsLPAAT2 (SEQ ID NO: 14), CiLPAAT2 (SEQ ID NO: 15), VaLPAAT2 (SEQ ID NO: 21), VcLPAAT2 (SEQ ID NO: 20), NoLPAT4 (SEQ ID NO: 17), NoLPAT3 (SEQ ID NO: 16), PmLPAAT1 (SEQ ID NO: 23), Sll1848 (SEQ ID NO: 19), BnBAT2 (SEQ ID NO: 18), and CrLPAAT1 (SEQ ID NO: 12).

In some embodiments, a microalgal cell provided herein comprises an exogenous gene that encodes for a LPAAT in Table 25. In some embodiments, the exogenous gene can comprise a sequence that encodes for an enzyme with at least 50%, at least 60%, at least 70%, at least 80%, at least 90%, at least 95%, at least 96%, at least 97%, at least 98%, at least 99%, or 100% sequence identity to a LPAAT in Table 25. In some embodiments, the exogenous gene can comprise a sequence that encodes for an enzyme with at least 50%, at least 60%, at least 70%, at least 80%, at least 90%, at least 95%, at least 96%, at least 97%, at least 98%, at least 99%, or 100% sequence identity to any one of SEQ ID NO: 12-24, 63, and 64.

TABLE 25
LPAAT Amino Acid Sequences

| SEQ ID | | |
|---|---|---|
| NO: | LPAAT | Sequence |
| 12 | CrLPAAT1 | MARKSSLAQAAIERKPVLLRPQLNVPRMSGITALPMERRP |
| | | LPVAPSPSAKPELPARSALVCHAAAASVPLPNSDSAPQPN |
| | | VLLAKIRAIMFFAWSFLLSLPLFVTMMVMAPLVLAFDKY |
| | | RRLAQHFVNNLWACASTAPFYKVTIIGRENLPPPDKPVVY |
| | | VANHQSFLDIYSLFHLQRPFKFISKTSNFLIPIIGWSMFLTG |
| | | HVMINRVDRRSQLKCLQQCRDLLAEGAPVLFFPEGTRSL |
| | | DCKMAGFKKGAFSVAAKAGVEVVPITLLGTGSLMPSGKE |
| | | SQLRPGQVTIVVHKALPPNKNADQLCDAARQAVASSLPP |
| | | ELVGSATEMAPDEQ |
| 13 | CrLPAAT2 | MSVLTKWLGLPSFLFSVFVFYWSLPIFAILYRIRFASLGKR |
| | | NDMLDWARALVAYFRVTLLQAGEHTLYKGGPCLYLCNH |
| | | RSWADFFIDAYLTEGRAALMSRWLVYFVFPVFCTSCMIL |
| | | KGIVLFKRGTIADKEAFNAWLDQTLGSSHVPGLLVYPEG |
| | | HRSTKPASLPLKRGMLHYAHSRKLPVQIVVTRGKDEVLS |
| | | EKSQSVHFGRTCVTTFSKVLKSADYPNFEAFFTDLQATW |
| | | DSCWAATYGLEDLKNVPRFSMPGPQAYSYSSSMWVQQL |
| | | AITLVSILVFAGVCYGSWRGLAAALAATGAAQQVVALVL |
| | | AAWVGSSVLRSFL |
| 14 | ChsLPAAT2 | MSALTKLLGLPSFLFSVFVFYWSLPIFAIFYRIRFASLGKRN |
| | | DMLDWARSLVGFFRVTLLQAGEQGLYKGGPCLYLCNHR |
| | | SWADFFVDAYLTEGRAALMSRWLVYFVFPVFCTSCMILK |
| | | GIVLFKRGTIADKEAFNAWLDRTLGDSHVPGLLVYPEGH |
| | | RSTKPTSLPLKRGMLHYAHSRKLPVQVIVTRGKDEVMSE |
| | | KGQSVHFGRTCVTTFSKVLKPADYPNFEAFFADLQATWD |
| | | ACWAATYGLKDLKGVPRFSMPSPQAYTYSAKTWAQQLA |
| | | ITLVSIVVFAAVCYGSWRGLAAALAATGAAQQVRGQQQ |
| | | QQQQQQRRRRLSSSGGWAEGRGAQAGGRLQWDGGDWG |
| | | RWVALPLSLEVVTVLLAAWVGGSVLRSFL |
| 15 | CILPAAT2 | MSALTKLLGLPSFLFSVFVFYWSLPIFAILYRIRFASLGKRN |
| | | DMLDWARALVGFFRVTLLQAGEQSLYKGGPCLYLCNHR |
| | | SWADFFIDAYLTEGRAALMSRWLVYFVFPVFCTSCMVLK |
| | | GIVLFKRGTIADKEAFNAWLDRTLGDSHVPGLLVYPEGH |
| | | RSTKPASLPLKRGMLHYAHSRKLPVQVIITRGKDEVLSEK |
| | | SQSVHFGRTCVTTFSKVIKPSDFPNFEAFFADLQATWDAG |
| | | WTATYGLKDLKGVPRFSMPSPQAYTYSSKMWAQQLAIT |
| | | LFSIPVFAGVCYGSWRVLAAALAATGAAQQVVTLALAG |
| | | WVGGSVLRSFL |
| 16 | NOLPAT3 | MWGLLWKHMTLRLLFVSVCLVTSSIGANMMALALFLVI |
| | | RPFSRSLYRRLVSQCVACMWIDALSLLLPGTNIHIAADSD |
| | | MPDGITAGIVVANHQYEGDWWFMLMVARFLGLHGNVKI |
| | | IVREGLKKIPLLGWLVRLVEYPFISSSWSLSRTNLFGLLRSF |
| | | NADDFPVLLFQFPEGDRIDAKVRQQSLAFAAKEQRPHLLH |
| | | VLLPRTTGFNTCIEALRTSHPPVYDMTIAIPGTTGQPSSSSS |
| | | SSSPSAAAAAASPSAGAAAAAAAASNTSTNPSSNPPSSSST |
| | | DTSPAAAAAAAADAAAAESHDASLFHTFLRFCNGEGPRD |
| | | VHIRLKRYSLNDVLADPHWLDTKWAEKDRVLTYFSRHA |
| | | CFPAPLPPHMANGGGSTQTHGGRGGPRGPAGSHAVVGA |
| | | AGNAQYLRSFNSRKFKAETSFLALARLLLTPLCLPLLLLM |
| | | ACPLMTLYVCVSTVRRWLGEEIVFSPGGREGGREGDAVY |
| | | ESVNWSV |
| 17 | NOLPAT4 | MSGPADARGPARVSDTIIGAPDRHHVGLTILVALLLSAFC |
| | | FCEFFMLPVAMALLPVPGTTSRKLLRRWEGLVAKTWLSF |
| | | GGWLLENVGGVKLILTGDSILPGDRVLILSNHRTRIDWMF |
| | | LWCWAARFDLLSSYRVILKSSLRTFPWWGWGMSLCLFPF |
| | | IHRGSNNRDADLTRIEHICQYLSELGVPNSLILFPEGTDLSQ |
| | | SNQKRDRDYALAKNLPIYHNVLHPRSGAFIASLTAMRPHL |
| | | DAVVDLTIGYVDYTPGERPSELSLLKGRLPHEVHINMKR |
| | | WDIKTTPLLRENPSDRAEQFLRESFDRKEALLTAFYSNNT |
| | | DTSGGGLKQTSSLLVSSSSSPFSNSSAPLGFPSFSSPSTSVA |
| | | SSYNEPVYVHNAHWTYGKGLLGTGIAMALVLMLACCLP |
| | | AVAFCGYSIVVFLLFVTVNSVWEGFDTLELNLAVWWGG |
| | | QAGQTKQE |
| 18 | BnBAT2 | MDVASARGVSSHPPYYSKPICSSQSSLIRIPISKGCCFARSS |
| | | NLITSLHAASRGVTRRTSGVQWCYRSIRFDPFKVNDKNSR |
| | | TVTVRSDLSGAATPESTYPEPEIKLSSRLRGICFCLVAGISA |
| | | IVLIVLMIIGHPFVLLFDRYRRKFHHFIAKLWASISIYPFYK |
| | | TDIQGLENLPSSDTPCVYVSNHQSFLDIYTLLSLGQSYKFIS |
| | | KTGIFVIPVIGWAMSMMGVVPLKRMDPRSQVDCLKRCM |
| | | ELVKKGASVFFFPEGTRSKDGRLGPFKKGAFTIAAKTGVP |
| | | VVPITLMGTGKIMPTGSEGILNHGDVRVIIHKPIYGSKADV |
| | | LCEEARNKIAESMNLLS |
| 19 | S111848 | MDSEINHRGGLSAPRPRETSLNLALYRGLKWGVVRPLLH |
| | | GLFQAQVYGQELVPTRGPALVVSNHASYFDPPFLSCAMA |
| | | RPVAFMAKEELFNVPLLGPAIRLYGAYPVKRGSGDRGAL |
| | | RAALTALGDGWLVGVFLEGTRTKDGRIHQPKLGAAMIA |
| | | AKAQVPIIPVSLGGVEQIFQPGSPWPHPVPLTIRIGKAIAPP |
| | | VKNRKPELEAVTKACQAQIHEMLDLGRD |
| 20 | VcLPAAT2 | MSVPSKALSLPSFLFSVFVFYWSLPLFAIIYRIRFANVGKR |
| | | NDMLDWWMVFFAFPVFCTSVLILRGIVLFKRGHIADKEL |
| | | LKSDAVVVMVVMVVATVVQAFNVWLDATLRKSTVPGL |
| | | LVYPEGHRSLKPRSLPLKRGMLHFAFSRHLAVQLVITRGK |
| | | EEVMSEKTCSARWGRTLVTNYSKVCAGGRWGGVIKPTD |
| | | FDSFDAFFNEIQTTWDACWQLSYHQPIAGVPRLSLKDAHE |
| | | YDYPPSMRWLQLGVTLLSIVILAAVLYGSWVVLRAGVLS |
| | | LGSSVTQQAVVVLGGAGGWLGLSLLRSFL |
| 21 | VaLPAAT2 | MSVIRKALSLPSFLFSVFVFYWSLPFFAIIYRIRFINVGKRN |
| | | DMLDWAKALVAFFHVTVLKQGEMTLYRGGRCLYLCNH |
| | | RSWADFFIDTYLTEGRAALMSRWLVYFVFPVFCSSVIVLR |
| | | GIVLFKRGHIANKEAFNAWLDVTLGKSTVPGLLVYPEGH |
| | | RSLKPTSLPLKRGMLHFAYSRKLPVQIIVTRGKEEVLSEKT |
| | | LSARWGRTLVVTFSHVLKPSDYDSFDAFFDDIQHKWDAC |
| | | WKSSYDKPTAGVPELSMKGAHEYDYPRSMWWLQLGMT |
| | | LMSIVILIGVMWGSWVALRTGLAVLGAAQQPVVLCAAV |
| | | WIGASMLRAFF |
| 22 | EcPlsC | MLYIFRLIITVIYSILVCVFGSIYCLFSPRNPKHVATFGHMF |
| | | GRLAPLFGLKVECRKPTDAESYGNAIYIANHQNNYDMVT |
| | | ASNIVQPPTVTVGKKSLLWIPFFGQLYWLTGNLLIDRNNR |
| | | TKAHGTIAEVVNHFKKRRISIWMFPEGTRSRGRGLLPFKT |
| | | GAFHAAIAAGVPIIPVCVSTTSNKINLNRLHNGLVIVEMLP |
| | | PIDVSQYGKDQVRELAAHCRSIMEQKIAELDKEVAEREA |
| | | AGKV |
| 23 | PmLPAAT1 | MAREGLDSVETADGGAKPENGAGEDVREGRSSPSDEDEA |
| | | LPAELEGLYPPEAPIHGPVDAGFAERVMTLQAKHDLAGK |
| | | GDALLKGTLEETTQLRLRKGEAGSEAGPAGSGSLYELARS |
| | | SPFIDLQHPIDAYELAKMVFLAPLFLFRALILLVLVASAYL |
| | | SVMVMFHVLPAGAHGLYPGRDALAQLLIHRCCPILLFLSG |
| | | FYHIPIKGAENIQKAKEARSIVIFNHLSYVDPAVLLCLMPL |
| | | SGIAKAGVARIPFIGPLGVALRWLFVERRGSSDRSNPHTLK |
| | | GDPIDAIRARRADHSAPLLVMAPEATTKAQQCLLKFRRG |
| | | AFTSGCPVTPVLIRYRFRHFNPAWGLCRSTPLHVYRLLVQ |
| | | PVNHVSLEVLPPYFPDEQETKDWTRFASNVRVRMAQALQ |
| | | APMVEQGIAEERRLLKAGVTTDLAGSRIIFRRKVAEQATS |
| | | PRKRD |
| 24 | PmLPAAT2 | MTLAASIKLALRWPLFLLSMALLYWSVVVGSLGGYIKFL |
| | | DLKSEQRGDLFAWSVAIKQAFGIRVLRVGKRDVYRGKC |
| | | MYLVSWGMMAVVFPLFMIALRAMRAVLVFNRGQIPDKE |
| | | KFNNWIDKELANTPQTGLAVYPEGHRSVLSDSLPLKRGM |
| | | LHYAYSRKMTVQCIICANKEAVLSEKYQVARWGQTVWA |
| | | GYSDPINAADFDSFEAFMTTVQSTWDTLWKEVYGADPTG |
| | | LPDLEMDDPVLDTPAHLRYAVSASVVLVDVVFFLVLWFG |
| | | ARFAIWATSPLGPLRGYLLFAAILTVVASFVVYSEPFNSVA |
| | | YNARKHQRRAAADPNRVRATGASISDKPAGVAPPTDLVA |
| | | HSVKDVTGDSLLVDGELDSKKDA |
| 63 | OILPAAT2 | MAPVFALSFQLSIFASTDGARVDRRARRSPGFEWGKFMT |
| | | RSVANARFKLAPGSGTLAREKRRTVWLCNHRAWSDFFL |
| | | DIYLTEGRAFTMSRMLVAYAFPLFMVPAMVCGVVFGFKR |
| | | DAGDKEKLNAALDAHFDAFGGEFAGMVCYPEGTRNVRE |
| | | NSMPLKRGMLRYAHSRKMAVQCVCTARKERVFSSHLTC |
| | | AERGLALPVYFAEVIHAEDYPDFEDFYNEIRSRWDNAWS |
| | | AANGVSAAEAESLPDYTPREHVVRCTPRHDAVIVSSALLT |
| | | VLGVALTWRACSKLL |
| 64 | CosLPAAT2 | MGVLEKIQTLPAFFLGVFFLYWSLPLAALMNKVKFLKLA |
| | | GKRNDAYGWGVALQQLFGVTMLRLPGPQLANPCGPIIFL |
| | | SNHRSWADFYLDVVATEGNAQMLSRMAVALVFPMFMG |
| | | AVCVIRSVILFQRGRNHEKEAFNNMIDRKMAASPVDGLIV |
| | | YPEGHRSTLRHSLPLKRGMLNYAYIRKIPVQVIFCNAVLC |
| | | LL |

### Example 13: Expressing Volvox carteri f. nagariensis LPAAT2 (VcLPAAT2 V2) in P. moriformis Strain CHK22

VcLPAAT2 V2 was introduced into a P. moriformis strain CHK22. The expression construct pCHK967 contained 5′ and 3′ homology arms to permit targeted integration of VcLPAAT2 V2 into the genome and is shown in Table 27.

TABLE 27
Integrative sequences for the transformation of P. moriformis with pCHK967
encoding VcLPAAT2 V2.

| SEQ | |
|---|---|
| ID | |
| NO: | Nucleotide Sequence |
| 25 | gctcttccgcttgcccgcaccctcgttgatctgggagccctgcgcagccccttaaatcatctcagtcaggtttctatgttcaactgagcct |
| | aaagggctttcgtcacgcgcacgagcacacgtatatcggctacgcagtctctcagaagcggtagaacagttcgcaagccctcgtcgg |
| | tcgaaaacttgcgccagtactattgaattaaattaaatgatcgaatgagacgcgaaacttttgcagaatgccactgagtttgcccaga |
| | gaatgggagtggcgccattcaccatccgcctgtgcacggcctgattcgccgagacgatgaatggcgagaccagggagcggcttgcg |
| | agccccgagccggtagcaggaataacggtcgacaatcttcctgtccaattactggcaaccattagaaagagccggagcgcgttgaa |
| | attctgcaatcgagtaatttttcgatgcggcgggcctgctgaaccctaaggctccggcctatgtttaaggcgatccaagatgcacgcgg |
| | ccccaggcacgtgtctcaagcacaaaccccagccttagtttcgagactttgggagatagcggcagatatctagtttggcattttgtatat |
| | taattaagtctcgcaatggagcgctctgatgcggtgcagcgtcggctgcagcacctggcagtggcgctggggtcgccctatcgctcgg |
| | 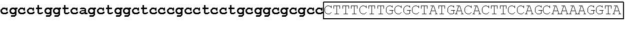 |
| | 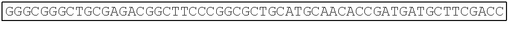 |
| | 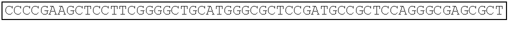 |
| | 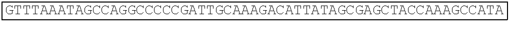 |
| | 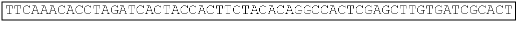 |
| | 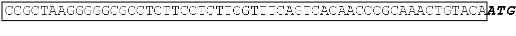 |
| | ctgctgcaggccttcctgttcctgctggccggcttcgccgccaagatcagcgcctccatgacgaacgagacgtccgaccgccccctggtg |
| | cacttcacccccaacaagggctggatgaacgaccccaacggcctgtggtacgacgagaaggacgccaagtggcacctgtacttccagt |
| | acaacccgaacgacaccgtctgggggacgcccttgttctggggccacgccacgtccgacgacctgaccaactgggaggaccagccca |
| | tcgccatcgccccgaagcgcaacgactccggcgccttctccggctccatggtggtggactacaacaacacctccggcttcttcaacgaca |
| | ccatcgacccgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctggtacgagc |
| | cctcccagaagtggatcatgaccgcggccaagtcccaggactacaagatcgagatctactcctccgacgacctgaagtcctggaagctg |
| | gagtccgcgttcgccaacgagggcttcctcggctaccagtacgagtgccccggcctgatcgaggtccccaccgagcaggaccccagca |
| | agtcctactgggtgatgttcatctccatcaaccccggcgccccggccggcggctccttcaaccagtacttcgtcggcagcttcaacggcac |
| | ccacttcgaggccttcgacaaccagtcccgcgtggtggacttcggcaaggactactacgccctgcagaccttcttcaacaccgacccgac |
| | ctacgggagcgccctgggcatcgcgtgggcctccaactgggagtactccgccttcgtgcccaccaacccctggcgctcctccatgtccctc |
| | gtgcgcaagttctccctcaacaccgagtaccaggccaacccggagacggagctgatcaacctgaaggccgagccgatcctgaacatc |
| | agcaacgccggcccctggagccggttcgccaccaacaccacgttgacgaaggccaacagctacaacgtcgacctgtccaacagcacc |
| | ggcaccctggagttcgagctggtgtacgccgtcaacaccacccagacgatctccaagtccgtgttcgcggacctctccctctggttcaagg |
| | gcctggaggaccccgaggagtacctccgcatgggcttcgaggtgtccgcgtcctccttcttcctggaccgcgggaacagcaaggtgaag |
| | ttcgtgaaggagaacccctacttcaccaaccgcatgagcgtgaacaaccagcccttcaagagcgagaacgacctgtcctactacaagg |
| | tgtacggcttgctggaccagaacatcctggagctgtacttcaacgacggcgacgtcgtgtccaccaacacctacttcatgaccaccggga |
| | acgccctgggctccgtgaacatgacgacgggggtggacaacctgttctacatcgacaagttccaggtgcgcgaggtcaagtgactTA |
| | AGACGCCCGCGCGGCGCACCTGACCTGTTCTCTCGAGGGCGCCTGTTCTGCCTTGCGA |
| | AACAAGCCCCTGGAGCATGCGTGCATGATCGTCTCTGGCGCCCCGCCGCGCGGTTTGT |
| | CGCCCTCGCGGGCGCCGCGGCCGCGGGGGCGCATTGAAATTGTTGCAAACCCCACCT |
| | GACAGATTGAGGGCCCAGGCAGGAAGGCGTTGAGATGGAGGTACAGGAGTCAAGTA |
| | ACTGAAAGTTTTTATGATAACTAACAACAAAGGGTCGTTTCTGGCCAGCGAATGACA |
| | AGAACAAGATTCCACATTTCCGTGTAGAGGCTTGCCATCGAATGTGAGCGGGCGGGC |
| | CGCGGACCCGACAAAACCCTTACGACGTGGTAAGAAAAACGTGGCGGGCACTGTCCC |
| | TGTAGCCTGAAGACCAGCAGGAGACGATCGGAAGCATCACAGCACAACCGGtgcagcag |
| | cagctcggatagtatcgacacactctggacgctggtcgtgtgatggactgttgccgccacacttgctgccttgacctgtgaatatccctgcc |
| | gcttttatcaaacagcctcagtgtgtttgatcttgtgtgtacgcgcttttgcgagttgctagctgcttgtgctatttgcgaataccacccccagca |
| | tccccttccctcgtttcatatcgcttgcatcccaaccgcaacttatctacgctgtcctgctatccctcagcgctgctcctgctcctgctcactgcc |
| | cctcgcacagccttggtttgggctccgcctgtattctcctggtactgcaacctgtaaaccagcactgcaatgctgatgcacgggaagtagtg |
| | 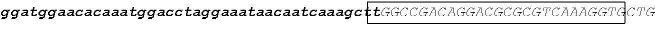 |
| | 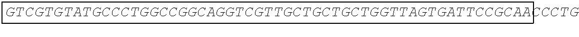 |
| | 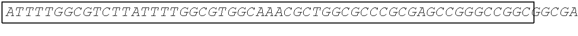 |
| | 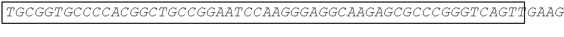 |
| | 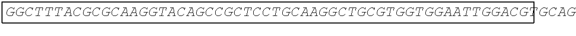 |
| | 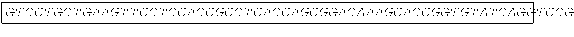 |
| | 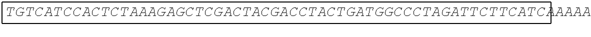 |
| | 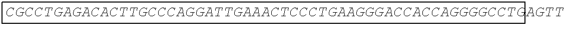 |
| | 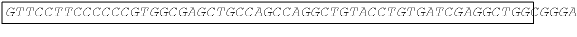 |
| | 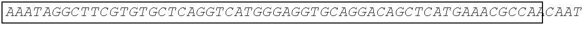 |
| | 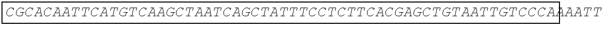 |
| | 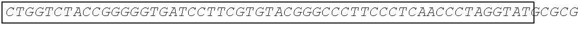 |
| | 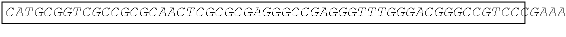 |
| | 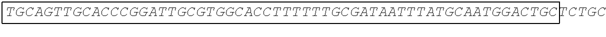 |
| | 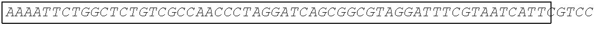 |
| | 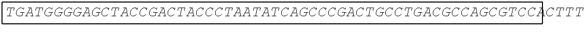 |
| | 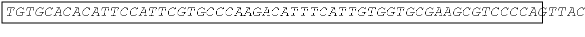 |
| | 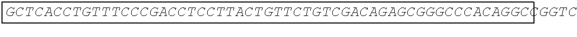 |
| | 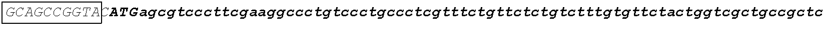 |
| | tttgcgattatctaccgtatccgcttcgccaacgtcggcaagcgcaacgacatgctggactgggcgaaggccctggtcgcttttttccgcgt |
| | gacggtgctgaagcagggcgagatgtccctctaccgtggaggccgctgcctgtacctctgtaaccatcgctcctgggccgactttttcattg |
| | atacctacctcaccgagggccgcgcggccctgatgtcccgatggatggtctttttcgcctttccggtgttctgcacctctgtcctcatcctgcgc |
| | ggcatcgtcctgttcaagcgaggccatatcgcggacaaggagacggcgtcgctgcaacccattgaggaggctttcaacgtctggctcga |
| | cgcgaccctccgcaagtccaccgtgccgggcctcctggtctacccggagggccatcggtccctcaagccccgctccctgccgctgaagc |
| | gcggaatgctgcacttcgctttctctcgccacctggctgtccaactgattatcacgcgaggtaaggaggaggtgatcaagccgacggattt |
| | cgactccttcgacgcgtttttccaggaggtgcagacgacctgggacgcctgctggcagctcagctaccaccagcccacggagggcgtgc |
| | cgcgcctgagcctcaaggatgcgcacgagtacgactacccgccctcgatgtggtggctgcaactgggcgtcaccctcctgtcgattgtgct |
| | cctggcggccgtgctgtacggctcctggctggtgctgcgcgctggcatcctgtcgctgggatctagcggcacccaacagacgcatcgcta |
| | cgtgagcgacgtccctgacctcgatcaactgaccgccctccaggagttcatgggcctgccgggcgacgtggacctccaggactacgctg |
| | acgtccccgagcggcgcatgacgctggtcctggacctggtggtcggccggacggaggagagcgcgaagggctgttgccagaagaag |
| | cgcgcggtctttgagatctcccgctaccgcgactacgcggccctgtgcacggagggctcgcaggtgatgcacgccctggacggcgtgat |
| | cacggacctgagcaacgggacctgcgactcgacggagtctgtggagcgtatcagcgccctgcccgctgagggcgtcggggagacgg |
| | gtgtggagggcgcgcgacggctgctcaactacattagctttttcggcaccccgctcggcgagggcgcgtggcaggcctgccgcacggtg |
| | atggaccgacgcgctcgcgctgcctccatcctcgacgagcccttcaccctggaggaggtggctcgcgcggtggggggctcccgaacg |
| | gcaaggcgcacggcctggaggtggccccgagcgagtgctaccgctacgcccgcctgccccgtgtggacgagcagggccgccagggc |
| | accgaggtcaactgcatcgtcccggtcctgcatgtgctgctcgcccatatccgcaccacgggcgacttcccccaacagtttacgaagacg |
| | gtggtcagccctattctgaagaagggcgacccgctggtcgcgggcaactactggggaatcgccgtcggtggcgccttcgccaagtgttac |
| | gcgtcggtgattctcaaccgactcgtccgcgcgggcgagacgctggacctccggcacccctgccaagcgggcttccgcctcgggtttgg |
| | aaccgcgcatcacctgttcgtgaagcagcacctggtgcggcgacgtcgctcggggggtgccccgccctgcgctgtcggcgcctgcccc |
| | gccggTGAcacgcccgcgcggcgcacctgacctgttctctcgagggcgcctgttctgccttgcgaaacaagcccctggagcatgcgtgc |
| | atgatcgtctctggcgccccgccgcgcggtttgtcgccctcgcgggcgccgcggccgcgggggcgcattgaaattgttgcaaaccccacct |
| | gacagattgagggcccaggcaggaaggcgttgagatggaggtacaggagtcaagtaactgaaagtttttatgataactaacaacaaagggtc |
| | gtttctggccagcgaatgacaagaacaagattccacatttccgtgtagaggcttgccatcgaatgtgagcggggggccgcggacccgacaa |
| | aacccttacgacgtggtaagaaaaacgtggcgggcactgtccctgtagcctgaagaccagcaggagacgatcggaagcatcacagcacaa |
| | ccggtatcgatagcgtctgcgtgttgggagctggagtcgtgggcttgacgacggcgctgcagctcttgcaggatgtgcctggcgtgcg |
| | cgttcacgtcgtggctgagaaatatggcgacgaaacgttgacggctggggccgggggctgtggatgccatacgcattgggtacgcg |
| | gccattggatgggattggtaggcttatggagggataatagagtttttgtcggatccaacgcatgttgatgcagtagcccggtgtgctga |
| | aagtatggaaggatggtgcattggctattcacatgcgctgcccaccccttttcgcaggaaatgtgccggcatcgttggtgcaccgatg |
| | gggaaaatcgacgttcgaccactacatgaaaatgtatacctctgaagatgcagcaactgcgggtgcgaaacggataacggtttggta |
| | ggtgtatgtcgcagcatgtgctggattttgcgggcaactccccctgccacggcccattgcaggtgtcatgttgactggaggatacgaac |
| | tttcggccgtcaaattcccagaggaggaccccctctgggccgacattgtgcccactgctcttccgcttgctgtttcctgtgtgaaattgt |

The construct can be written as 5′DAO1b::CrTUB2:ScSUC2:PmPGH:CvNR:PmAMT03:VcLPAAT2 V2:PmPGH::3′DAO1b. Proceeding in the 5′ to 3′ direction, bold, lowercase sequences represent genomic DNA from CHK22 that permit targeted integration at the DAO1b locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text that enables amplification of the VcLPAAT2 V2 gene, followed by a linker, indicated by lowercase, bold italics, in which contains a C. vulgaris nitrate reductase 3′-UTR. The P. moriformis PmAMT03 promoter, indicated by boxed italicized text, drives the expression of the VcLPAAT2 V2. The initiator ATG and terminator TGA codons of the CrLPAAT2 V2 are indicated by uppercase, bold italics, while the remainder of the gene is indicated by bold italics. The P. moriformis PmPGH 3′-UTR is indicated by lowercase underlined text followed by the CHK22 DAO1b genomic region indicated by bold, lowercase text.

Table 28 shows the fatty acid/TAG profiles of primary transformants of CHK22 transformed with pCHK967. P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMES for subsequent quantitation and characterization by GC/FID. TAG analysis was conducted using the LCMS method as described above.

TABLE 28
Screen of primary transformants of CHK22 with pCHK967.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK967- | 0.05 | 1.62 | 21.57 | 3.09 | 58.33 | 12.72 | 0.61 | 0.28 | 0.11 | 1.55 |
| 1 | | | | | | | | | | |
| pCHK967- | 0.05 | 1.61 | 24.49 | 2.75 | 59.06 | 9.64 | 0.56 | 0.26 | 0.09 | 1.45 |
| 2 | | | | | | | | | | |
| pCHK967- | 0.05 | 1.64 | 22.82 | 3.13 | 58.83 | 11.12 | 0.56 | 0.31 | 0.10 | 1.53 |
| 3 | | | | | | | | | | |
| pCHK967- | 0.05 | 1.59 | 22.40 | 3.26 | 58.98 | 11.26 | 0.57 | 0.30 | 0.10 | 1.57 |
| 4 | | | | | | | | | | |
| pCHK967- | 0.06 | 1.68 | 22.50 | 3.31 | 58.05 | 11.91 | 0.60 | 0.30 | 0.10 | 1.57 |
| 5 | | | | | | | | | | |
| CHK22 | 0.04 | 1.56 | 26.48 | 3.06 | 58.93 | 7.59 | 0.47 | 0.27 | 0.07 | 1.56 |
| Control | | | | | | | | | | |

### Example 14: Expressing Astrephomene gubernaculifera LPAAT2 (AgLPAAT2) in P. moriformis Strain CHK22

AgLPAAT2 was introduced into a P. moriformis strain CHK22. The expression construct pCHK968 contained 5′ and 3′ homology arms to permit targeted integration of AgLPAAT2 into the genome and is shown in Table 29.

TABLE 29
Integrative sequences for the transformation of P. moriformis with pCHK968
encoding AgLPAAT2.

| SEQ | |
|---|---|
| ID | |
| NO: | Nucleotide Sequence |
| 26 | gctcttccgcttgcccgcaccctcgttgatctgggagccctgcgcagccccttaaatcatctcagtcaggtttctatgttcaactgagcct |
| | aaagggctttcgtcacgcgcacgagcacacgtatatcggctacgcagtctctcagaagcggtagaacagttcgcaagccctcgtcgg |
| | tcgaaaacttgcgccagtactattgaattaaattaaatgatcgaatgagacgcgaaacttttgcagaatgccactgagtttgcccaga |
| | gaatgggagtggcgccattcaccatccgcctgtgcacggcctgattcgccgagacgatgaatggcgagaccagggagcggcttgcg |
| | agccccgagccggtagcaggaataacggtcgacaatcttcctgtccaattactggcaaccattagaaagagccggagcgcgttgaa |
| | attctgcaatcgagtaatttttcgatgcggcgggcctgctgaaccctaaggctccggcctatgtttaaggcgatccaagatgcacgcgg |
| | ccccaggcacgtgtctcaagcacaaaccccagccttagtttcgagactttgggagatagcggcagatatctagtttggcattttgtatat |
| | taattaagtctcgcaatggagcgctctgatgcggtgcagcgtcggctgcagcacctggcagtggcgctggggtcgccctatcgctcgg |
| | 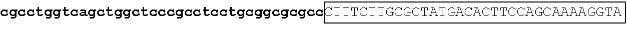 |
| | 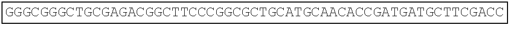 |
| | 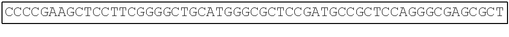 |
| | 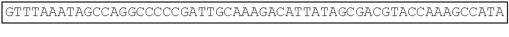 |
| | 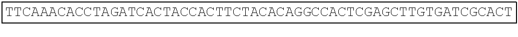 |
| | 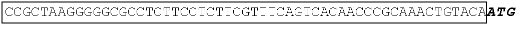 |
| | ctgctgcaggccttcctgttcctgctggccggcttcgccgccaagatcagcgcctccatgacgaacgagacgtccgaccgccccctggtg |
| | cacttcacccccaacaagggctggatgaacgaccccaacggcctgtggtacgacgagaaggacgccaagtggcacctgtacttccagt |
| | acaacccgaacgacaccgtctgggggacgcccttgttctggggccacgccacgtccgacgacctgaccaactgggaggaccagccca |
| | tcgccatcgccccgaagcgcaacgactccggcgccttctccggctccatggtggtggactacaacaacacctccggcttcttcaacgaca |
| | ccatcgacccgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctggtacgagc |
| | cctcccagaagtggatcatgaccgcggccaagtcccaggactacaagatcgagatctactcctccgacgacctgaagtcctggaagctg |
| | gagtccgcgttcgccaacgagggcttcctcggctaccagtacgagtgccccggcctgatcgaggtccccaccgagcaggaccccagca |
| | agtcctactgggtgatgttcatctccatcaaccccggcgccccggccggcggctccttcaaccagtacttcgtcggcagcttcaacggcac |
| | ccacttcgaggccttcgacaaccagtcccgcgtggtggacttcggcaaggactactacgccctgcagaccttcttcaacaccgacccgac |
| | ctacgggagcgccctgggcatcgcgtgggcctccaactgggagtactccgccttcgtgcccaccaacccctggcgctcctccatgtccctc |
| | gtgcgcaagttctccctcaacaccgagtaccaggccaacccggagacggagctgatcaacctgaaggccgagccgatcctgaacatc |
| | agcaacgccggcccctggagccggttcgccaccaacaccacgttgacgaaggccaacagctacaacgtcgacctgtccaacagcacc |
| | ggcaccctggagttcgagctggtgtacgccgtcaacaccacccagacgatctccaagtccgtgttcgcggacctctccctctggttcaagg |
| | gcctggaggaccccgaggagtacctccgcatgggcttcgaggtgtccgcgtcctccttcttcctggaccgcgggaacagcaaggtgaag |
| | ttcgtgaaggagaacccctacttcaccaaccgcatgagcgtgaacaaccagcccttcaagagcgagaacgacctgtcctactacaagg |
| | tgtacggcttgctggaccagaacatcctggagctgtacttcaacgacggcgacgtcgtgtccaccaacacctacttcatgaccaccggga |
| | acgccctgggctccgtgaacatgacgacgggggtggacaacctgttctacatcgacaagttccaggtgcgcgaggtcaagtgactTA |
| | AGACGCCCGCGCGGCGCACCTGACCTGTTCTCTCGAGGGCGCCTGTTCTGCCTTGCGA |
| | AACAAGCCCCTGGAGCATGCGTGCATGATCGTCTCTGGCGCCCCGCCGCGCGGTTTGT |
| | CGCCCTCGCGGGCGCCGCGGCCGCGGGGGCGCATTGAAATTGTTGCAAACCCCACCT |
| | GACAGATTGAGGGCCCAGGCAGGAAGGCGTTGAGATGGAGGTACAGGAGTCAAGTA |
| | ACTGAAAGTTTTTATGATAACTAACAACAAAGGGTCGTTTCTGGCCAGCGAATGACA |
| | AGAACAAGATTCCACATTTCCGTGTAGAGGCTTGCCATCGAATGTGAGCGGGCGGGC |
| | CGCGGACCCGACAAAACCCTTACGACGTGGTAAGAAAAACGTGGCGGGCACTGTCCC |
| | TGTAGCCTGAAGACCAGCAGGAGACGATCGGAAGCATCACAGCACAACCGGtgcagcag |
| | cagctcggatagtatcgacacactctggacgctggtgtgtgatggactgttgccgccacacttgctgccttgacctgtgaatatccctgccgc |
| | ttttatcaaacagcctcagtgtgtttgatcttgtgtgtacgcgcttttgcgagttgctagctgcttgtgctatttgcgaataccacccccagca |
| | tccccttccctcgtttcatatcgcttgcatcccaaccgcaacttatctacgctgtcctgctatccctcagcgctgctcctgctcctgctcactg |
| | cccctcgcacagccttggtttgggctccgcctgtattctcctggtactgcaacctgtaaaccagcactgcaatgctgatgcacgggaagtagtg |
| | 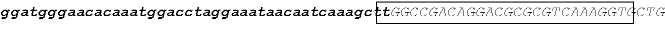 |
| | 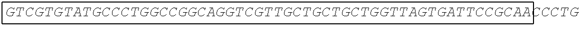 |
| | 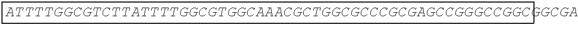 |
| | 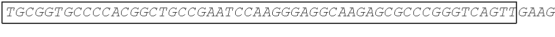 |
| | 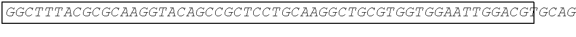 |
| | 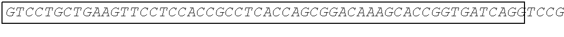 |
| | 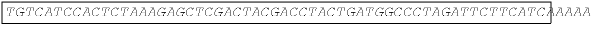 |
| | 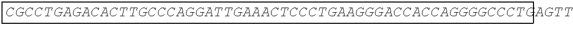 |
| | 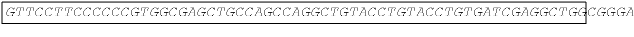 |
| | 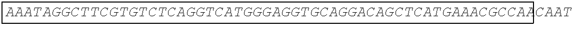 |
| | 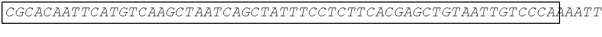 |
| | 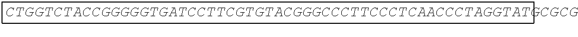 |
| | 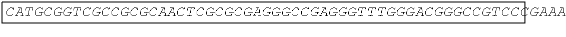 |
| | 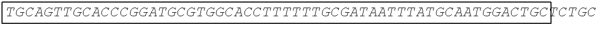 |
| | 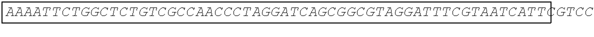 |
| | 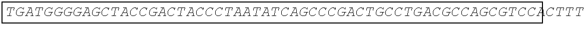 |
| | 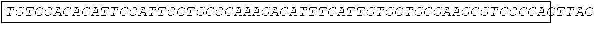 |
| | 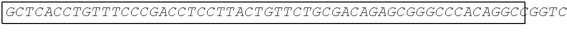 |
| | 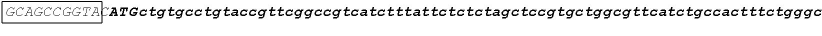 |
| | gcgctcggtgcgtgcgtgctctggggtcaactgagcctctcgtctcgcaacctcttcgggctccgctgcaactgctgtctgctcaccgacagca |
| | tggaggccagctcgaaggtcctgacgttccccgcgttcctctgctccgtgttcgtgttctactggtcgctgcctatctttgcgatcatttacc |
| | gcatccggtttgcgaacctgggaaagcgcaacgatatgctggagtgggcgaagtcgctggtccggttctttggcgtgacggtcctgaagg |
| | agggcgagtcttccctgtaccgcggagggaagtgcctgtacctgtgcaaccaccgtagctgggcggacttctttatcgacgcctacctgac |
| | ggaggggcgggcggccctgatgtcgcgctggctggtgtacttcgtgttccccgtgttttgtacgtcgtgcctgatcctgcggggcatcgtgct |
| | gttcaagcggggcaccatcgctgacaaggaggccttcaactcttggctcgatgccacgctgggtcggtcccatgtgccgggcctcctggt |
| | ctacccggagggccaccgttccctgaagcctgcgtcgctgccgctgaagcgcggcatgctgcactacgcgtttagccgcaagatccctgt |
| | ccagatcattatcacgcgaggtaaggaggaggtcctgtccgagaagcggatggccgtccgcttcggccggacgctggtgacgcagttct |
| | ccaaggtcatcgagccgggccaattcgccagcttcgacgcgtttttctcggccgtgcagagcacgtgggacgagtgctggcgcggcgcc |
| | tacggcgcgagcacgacctcgacgccccgccataccctgtcccacccacccacggagtacgactacccggcccgcatgcgcctgcaa |
| | caggccgcgatcacgctgttcagcatggtcgtgctggctggcgtgctgggggctcgtggggggggcgccgcggtcTAGcacgcc |
| | cgcgcggcgcacctgacctgttctctcgagggcgcctgttctgccttgcgaaacaagcccctggagcatgcgtgcatgatcgtctctggcgcc |
| | ccgccgcgcggtttgtcgccctcgcgggcgccgcggccgcgggggcgcattgaaattgttgcaaaccccacctgacagattgagggccca |
| | ggcaggaaggcgttgagatggaggtacaggagtcaagtaactgaaagtttttatgataactaacaacaaagggtcgtttctggccagcgaatg |
| | acaagaacaagattccacatttccgtgtagaggcttgccatcgaatgtgagcggggggccgcggacccgacaaaacccttacgacgtggt |
| | aagaaaaacgtggcgggcactgtccctgtagcctgaagaccagcaggagacgatcggaagcatcacagcacaaccggtatcgatagcgt |
| | ctgcgtgttgggagctggagtcgtgggcttgacgacggcgctgcagctcttgcaggatgtgcctggcgtgcgcgttcacgtcgtggct |
| | gagaaatatggcgacgaaacgttgacggctggggccggcgggctgtggatgccatacgcattgggtacgcggccattggatgggat |
| | tggtaggcttatggagggataatagagtttttgtcggatccaacgcatgttgatgcagtagcccggtgtgctgaaagtatggaaggat |
| | ggtgcattggctattcacatgcgctgcccaccccttttcgcaggaaatgtgccggcatcgttggtgcaccgatggggaaaatcgacgtt |
| | cgaccactacatgaaaatgtatacctctgaagatgcagcaactgcgggtgcgaaacggataacggtttggtaggtgtatgtcgcagc |
| | atgtgctggattttgcgggcaactccccctgccacggcccattgcaggtgtcatgttgactggaggatacgaactttcggccgtcaaat |
| | tcccagaggaggaccccctctgggccgacattgtgcccactgctcttccgcttgctgtttcctgtgtgaaattgt |

The construct can be written as 5′DAO1b::CrTUB2:ScSUC2:PmPGH:CvNR:PmAMT03:AgLPAAT2:PmPGH::3′DAO1b. Proceeding in the 5′ to 3′ direction bold, lowercase sequences represent genomic DNA from CHK22 that permit targeted integration at the DAO1b locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text that enables amplification of the AgLPAAT2 gene, followed by a linker, indicated by lowercase, bold italics, in which contains a C. vulgaris nitrate reductase 3′-UTR. The P. moriformis PmAMT03 promoter, indicated by boxed italicized text, drives the expression of the AgLPAAT2. The initiator ATG and terminator TAG codons of the AgLPAAT2 are indicated by uppercase, bold italics, while the remainder of the gene is indicated by bold italics. The P. moriformis PmPGH 3′-UTR is indicated by lowercase underlined text followed by the CHK22 DAO1b genomic region indicated by bold, lowercase text.

Table 30 shows the fatty acid/TAG profiles of primary transformants of CHK22 transformed with pCHK968. The P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as

TABLE 30
Screen of primary transformants of CHK22 with pCHK968.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK968- | 0.06 | 1.67 | 20.64 | 3.35 | 57.91 | 13.90 | 0.64 | 0.32 | 0.11 | 1.59 |
| 1 | | | | | | | | | | |
| pCHK968- | 0.07 | 2.09 | 19.46 | 2.71 | 51.79 | 21.09 | 0.82 | 0.23 | 0.17 | 2.21 |
| 2 | | | | | | | | | | |
| pCHK968- | 0.05 | 1.59 | 21.25 | 3.49 | 58.21 | 12.97 | 0.59 | 0.30 | 0.11 | 1.83 |
| 3 | | | | | | | | | | |
| pCHK968- | 0.05 | 1.53 | 20.67 | 3.25 | 58.74 | 13.26 | 0.57 | 0.31 | 0.11 | 1.53 |
| 4 | | | | | | | | | | |
| pCHK968- | 0.05 | 1.52 | 21.18 | 3.25 | 58.34 | 13.19 | 0.60 | 0.30 | 0.11 | 1.63 |
| 5 | | | | | | | | | | |
| pCHK968- | 0.05 | 1.54 | 24.14 | 3.28 | 58.66 | 9.94 | 0.53 | 0.30 | 0.08 | 1.64 |
| 6 | | | | | | | | | | |
| CHK22 | 0.04 | 1.56 | 26.48 | 3.06 | 58.93 | 7.59 | 0.47 | 0.27 | 0.07 | 1.56 |
| Control | | | | | | | | | | |

### Example 15: Expressing Edaphochlamys debaryana LPAAT2 (EdLPAAT2) in P. moriformis Strain CHK22

EdLPAAT2 was introduced into a P. moriformis strain CHK22. The expression construct pCHK969 contained 5Y and 3Y homology arms to permit targeted integration of EdLPAAT2 into the genome and is shown in Table 31.

TABLE 31
Integrative sequences for the transformation of P. moriformis with pCHK969
encoding EdLPAAT2.

| SEQ | |
|---|---|
| ID | |
| NO: | Nucleotide Sequence |
| 27 | gctcttccgcttgcccgcaccctcgttgatctgggagccctgcgcagccccttaaatcatctcagtcaggtttctatgttcaactgagc |
| | ctaaagggctttcgtcacgcgcacgagcacacgtatatcggctacgcagtctctcagaagcggtagaacagttcgcaagccctcgtcgg |
| | tcgaaaacttgcgccagtactattgaattaaattaaatgatcgaatgagacgcgaaacttttgcagaatgccactgagtttgcccaga |
| | gaatgggagtggcgccattcaccatccgcctgtgcacggcctgattgccgagacgatgaatggcgagaccagggagcggcttgcg |
| | agccccgagccggtagcaggaataacggtcgacaatcttcctgtccaattactggcaaccattagaaagagccggagcgcgttgaa |
| | attctgcaatcgagtaatttttcgatgcggcgggcctgctgaaccctaaggctccggcctatgtttaaggcgatccaagatgcacgcgg |
| | ccccaggcacgtgtctcaagcacaaaccccagccttagtttcgagactttgggagatagcggcagatatctagtttggcattttgtatat |
| | taattaagtctcgcaatggagcgctctgatgcggtgcagcgtcggctgcagcacctggcagtggcgctggggtcgccctatcgctcgg |
| | 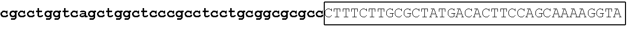 |
| | 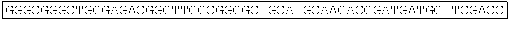 |
| | 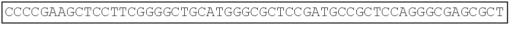 |
| | 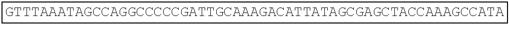 |
| | 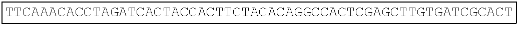 |
| | 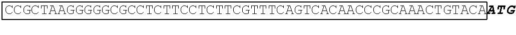 |
| | ctgctgcaggccttcctgttcctgctggccggcttcgccgccaagatcagcgcctccatgacgaacgagacgtccgaccgccccctggtg |
| | cacttcacccccaacaagggctggatgaacgaccccaacggcctgtggtacgacgagaaggacgccaagtggcacctgtacttccagt |
| | acaacccgaacgacaccgtctgggggacgcccttgttctggggccacgccacgtccgacgacctgaccaactgggaggaccagccca |
| | tcgccatcgccccgaagcgcaacgactccggcgccttctccggctccatggtggtggactacaacaacacctccggcttcttcaacgaca |
| | ccatcgacccgcgccagcgctgcgtggccatctggacctacaacacccggagtccgaggagcagtacatctcctacagcctggacgg |
| | cggctacaccttcaccgagtaccagaagccccgtgctggccgccaactccacccagttccgcgacccgaaggtcttctggtacgagc |
| | cctcccagaagtggatcatgaccgcggccaagtcccaggactacaagatcgagatctactcctccgacgacctgaagtcctggaagctg |
| | gagtccgcgttcgccaacgagggcttcctcggctaccagtacgagtgccccggcctgatcgaggtccccaccgagcaggaccccagca |
| | agtcctactgggtgatgttcatctccatcaaccccggcgccccggccggcggctccttcaaccagtacttcgtcaggcagcttcaacggcac |
| | ccacttcgaggccttcgacaaccagtcccgcgtggtggacttcggcaaggactactacgccctgcagaccttcttcaacaccgacccgac |
| | ctacgggagcgccctgggcatcgcgtgggcctccaactgggagtactccgccttcgtgcccaccaacccctggcgctcctccatgtccctc |
| | gtgcgcaagttctccctcaacaccgagtaccaggccaacccggagacggagctgatcaacctgaaggccgagccgatcctgaacatc |
| | agcaacgccggcccctggagccggttcgccaccaacaccacgttgacgaaggccaacagctacaacgtcgacctgtccaacagcacc |
| | ggcaccctggagttcgagctggtgtacgccgtcaacaccacccagacgatctccaagtccgtgttcgcggacctctccctctggttcaagg |
| | gcctggaggaccccgaggagtacctccgcatgggcttcgaggtgtccgcgtcctccttcttcctggaccgcgggaacagcaaggtgaag |
| | ttcgtgaaggagaacccctacttcaccaaccgcatgagcgtgaacaaccagcccttcaagagcgagaacgacctgtcctactacaagg |
| | tgtacggcttgctggaccagaacatcctggagctgtacttcaacgacggcgacgtcgtgtccaccaacacctacttcatgaccaccggga |
| | acgccctgggctccgtgaacatgacgacgggggtggacaacctgttctacatcgacaagttccaggtgcgcgaggtcaagtgactTA |
| | AGACGCCCGCGCGGCGCACCTGACCTGTTCTCTCGAGGGCGCCTGTTCTGCCTTGCGA |
| | AACAAGCCCCTGGAGCATGCGTGCATGATCGTCTCTGGCGCCCCGCCGCGCGGTTTGT |
| | CGCCCTCGCGGGCGCCGCGGCCGCGGGGGCGCATTGAAATTGTTGCAAACCCCACCT |
| | GACAGATTGAGGGCCCAGGCAGGAAGGOGTTGAGATGGAGGTACAGGAGTCAAGTA |
| | ACTGAAAGTTTTTATGATAACTAACAACAAAGGGTCGTTTCTGGCCAGCGAATGACA |
| | AGAACAAGATTCCACATTTCCGTGTAGAGGCTTGCCATCGAATGTGAGCGGGCGGGC |
| | CGCGGACCCGACAAAACCCTTACGACGTGGTAAGAAAAACGTGGCGGGCACTGTCCC |
| | TGTAGCCTGAAGACCAGCAGGAGACGATCGGAAGCATCACAGCACAACCGGtgcagcag |
| | cagctcggatagtatcgacacactctggacgctggtcgtgtgatggactgttgccgccacacttgctgccttgacctgtgaatatccctgccgc |
| | ttttatcaaacagcctcagtgtgtttgatcttgtgtgtacgcgcttttgcgagttgctagctgcttgtgctatttgcgaataccacccccagca |
| | tccccttccctcgtttcatatcgcttgcatcccaaccgcaacttatctacgctgtcctgctatccctcagcgctgctcctgctcctgctcactg |
| | cccctcgcacagccttggtttgggctccgcctgtattctcctggtactgcaacctgtaaaccagcactgcaatgctgatgcacgggaagtagtg |
| | 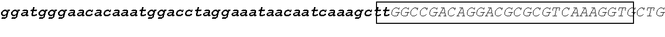 |
| | 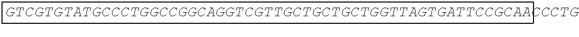 |
| | 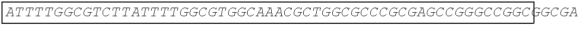 |
| | 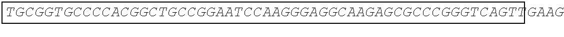 |
| | 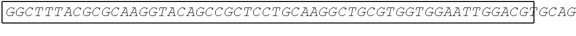 |
| | 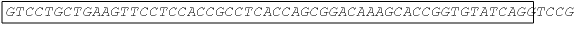 |
| | 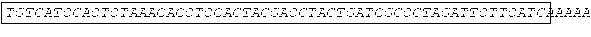 |
| | 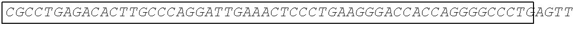 |
| | 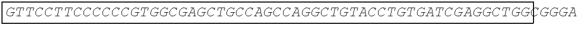 |
| | 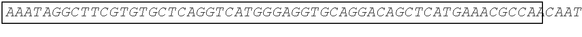 |
| | 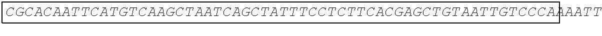 |
| | 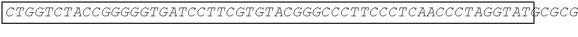 |
| | 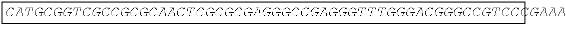 |
| | 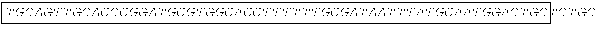 |
| | 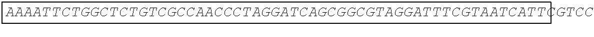 |
| | 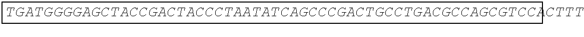 |
| | 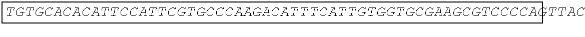 |
| | 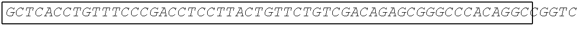 |
| | 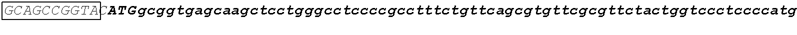 |
| | ctggcgatcacgtatcggctgcgcttcctgaacctcggcaagcgcaacgacatgctggactggggccgcggactcgtcaactttttcgctg |
| | tcaccatttacaaggtgggcgacggctcgctgtacaagggtggctcctgcctgtacctctgcaaccaccgctcttgggcggactttttcatcg |
| | acatctacatcacggaggggcgcggcgccatgatgagccgctggctcgtgtttttcgtgttccctatcttttgctccgccgtggtcaccctga |
| | agggcattatcctgtttaagcggggcaccatcgccgacaaggagaagtttaacgcctggctggacgcgacgctggcttcgagcccggtg |
| | ccgggcatgctggtgtacccggagggtcaccgttcgatcaagccccactcgctccccctgaagcgcggcatgctgcattacgcgttcagc |
| | cgcaagctgcccgtccaggtgatcattacccgtggcaaggatgacgtgctctcggagaagaccatggcggtgcattggcgacggcaact |
| | ggtgaccagctacggcaaggtgattaagccgagcgagtgcgcctcgttcgacgccttcatgcatgaggtgcagacggagtgggacaag |
| | cactgggcggaggcctacaacgcgcccaagcccccaagcaagagcctgcctcgcctcgtcccggccccgcatggctacgactaccca |
| | ctgagcatgcgcctgggtatgggttccgtgaccacgttctcgatcttcgccttcgcgggcgtcctgtacggcacctactgcacggctctgtgg |
| | gtgctcgcgtttacgggcgcggcccagaaggtgatcgtgacggtgctcgccctgtggatgggtctggccctcacccgcgcggtgctgTA |
| | Gcacgcccgcgcggcgcacctgacctgttctctcgagggcgcctgttctgccttgcgaaacaagcccctggagcatgcgtgcatgatcgtct |
| | ctggcgccccgccgcgcggttttgtcgccctcgcgggcgccgcggccgcgggggcgcattgaaattgttgcaaaccccacctgacagattg |
| | agggcccaggcaggaaggcgttgagatggaggtacaggagtcaagtaactgaaagtttttatgataactaacaacaaagggtcgtttctggcc |
| | agcgaatgacaagaacaagattccacatttccgtgtagaggcttgccatcgaatgtgagcgggcgggccgcggacccgacaaaacccttac |
| | gacgtggtaagaaaaacgtggcgggcactgtccctgtagcctgaagaccagcaggagacgatcggaagcatcacagcacaaccggtatc |
| | gatagcgtctgcgtgttgggagctggagtcgtgggcttgacgacggcgctgcagctcttgcaggatgtgcctggcgtgcgcgttcacg |
| | tcgtggctgagaaatatggcgacgaaacgttgacggctggggccggcgggctgtggatgccatacgcattgggtacgcggccattg |
| | gatgggattggtaggcttatggagggataatagagtttttgtcggatccaacgcatgttgatgcagtagcccggtgtgctgaaagtatg |
| | gaaggatggtgcattggctattcacatgcgctgcccaccccttttcgcaggaaatgtgccggcatcgttggtgcaccgatggggaaaa |
| | tcgacgttcgaccactacatgaaaatgtatacctctgaagatgcagcaactgcgggtgcgaaacggataacggtttggtaggtgtatg |
| | tcgcagcatgtgctggattttgcgggcaactccccctgccacggcccattgcaggtgtcatgttgactggaggatacgaactttcggcc |
| | gtcaaattcccagaggaggaccccctctgggccgacattgtgcccactgctcttccgcttgctgtttcctgtgtgaaattgt |

The construct can be written as 5′DAO1b::CrTUB2:ScSUC2:PmPGH:CvNR:PmAMT03:EdLPAAT2:PmPGH::3′DAO1b. Proceeding in the 5′ to 3′ direction bold, lowercase sequences represent genomic DNA from CIK22 that permit targeted integration at the DAO1b locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text that enables amplification of the EdLPAAT2 gene, followed by a linker, indicated by lowercase, bold italics, in which contains a C. vulgaris nitrate reductase 3′-UTR. The P. moriformis PmAMT03 promoter, indicated by boxed italicized text, drives the expression of the EdLPAAT2. The initiator ATG and terminator TAG codons of the EdLPAAT2 are indicated by uppercase, bold italics, while the remainder of the gene is indicated by bold italics. The P. moriformis PmPGH 3′-UTR is indicated by lowercase underlined text followed by the CHK22 DAO1b genomic region indicated by bold, lowercase text.

Table 32 shows the fatty acid/TAG profiles of primary transformants of CHK22 transformed with pCHK969. The P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 32
Screen of primary transformants of CHK22 with pCHK969.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK969- | 0.05 | 1.58 | 21.56 | 3.58 | 57.99 | 12.96 | 0.58 | 0.25 | 0.12 | 3.36 |
| 1 | | | | | | | | | | |
| pCHK969- | 0.05 | 1.60 | 21.25 | 3.51 | 57.37 | 13.94 | 0.56 | 0.22 | 0.12 | 3.78 |
| 2 | | | | | | | | | | |
| pCHK969- | 0.05 | 1.57 | 20.99 | 3.28 | 58.55 | 13.11 | 0.60 | 0.32 | 0.11 | 1.53 |
| 3 | | | | | | | | | | |
| pCHK969- | 0.05 | 1.51 | 22.39 | 3.25 | 59.01 | 11.41 | 0.56 | 0.31 | 0.10 | 1.55 |
| 4 | | | | | | | | | | |
| pCHK969- | 0.04 | 1.52 | 24.72 | 3.10 | 59.05 | 9.28 | 0.50 | 0.29 | 0.09 | 1.54 |
| 5 | | | | | | | | | | |
| pCHK969- | 0.05 | 1.68 | 26.00 | 3.09 | 57.68 | 9.20 | 0.51 | 0.29 | 0.09 | 2.19 |
| 6 | | | | | | | | | | |
| CHK22 | 0.04 | 1.56 | 26.48 | 3.06 | 58.93 | 7.59 | 0.47 | 0.27 | 0.07 | 1.56 |
| Control | | | | | | | | | | |

### Example 16: Expressing Dunaliella salina LPAAT2 (DsLPAAT2) in P. moriformis Strain CHK22

DsLPAAT2 was introduced into a P. moriformis strain CHK22. The expression construct pCHK971 contained 5Y and 3Y homology arms to permit targeted integration of DsLPAAT2 into the genome and is shown in Table 33.

TABLE 33
Integrative sequences for the transformation of P. moriformis with pCHK971 encoding DsLPAAT2.

| SEQ ID | |
|---|---|
| NO: | Nucleotide Sequence |
| 28 | gctcttccgcttghcccgcaccctcgttgatctgggagccctgcgcagccccttaaatcatctcagtcaggtttctatgttcaactgagcct |
| | aaagggctttcgtcacgcgcacgagcacacgtatatcggctacgcagtctctcagaagcggtagaacagttcgcaagccctcgtcgg |
| | tcgaaaacttgcgccagtactattgaattaaattaaatgatcgaatgagacgcgaaacttttgcagaatgccactgagtttgcccaga |
| | gaatgggagtggcgccattcaccatccgcctgtgcacggcctgattcgccgagacgatgaatggcgagaccagggagcggcttgcg |
| | agccccgagccggtagcaggaataacggtcgacaatcttcctgtccaattactggcaaccattagaaagagccggagcgcgttgaa |
| | attctgcaatcgagtaatttttcgatgcggcgggcctgctgaaccctaaggctccggcctatgtttaaggcgatccaagatgcacgcgg |
| | ccccaggcacgtgtctcaagcacaaaccccagccttagtttcgagactttgggagatagcggcagatatctagtttggcattttgtatat |
| | taattaagtctcgcaatggagcgctctgatgcggtgcagcgtcggctgcagcacctggcagtggcgctggggtcgccctatcgctcgg |
| | 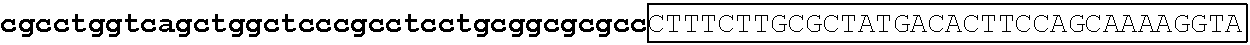 |
| | 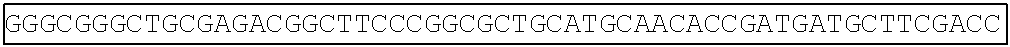 |
| | 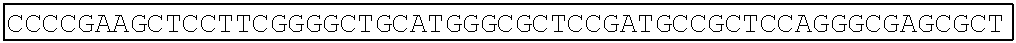 |
| | 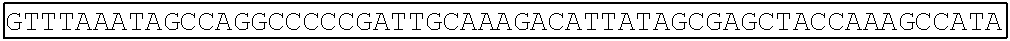 |
| | 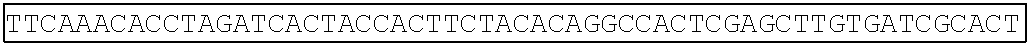 |
| | 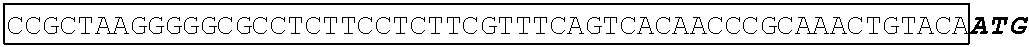 |
| | ctgctgcaggccttcctgttcctgctggccggcttcgccgccaagatcagcgcctccatgacgaacgagacgtccgaccgccccctggtg |
| | cacttcacccccaacaagggctggatgaacgaccccaacggcctgtggtacgacgagaaggacgccaagtggcacctgtacttccagt |
| | acaacccgaacgacaccgtctgggggacgcccttgttctggggccacgccacgtccgacgacctgaccaactgggaggaccagccca |
| | tcgccatcgccccgaagcgcaacgactccggcgccttctccggctccatggtggtggactacaacaacacctccggcttcttcaacgaca |
| | ccatcgacccgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctggtacgagc |
| | cctcccagaagtggatcatgaccgcggccaagtcccaggactacaagatcgagatctactcctccgacgacctgaagtcctggaagctg |
| | gagtccgcgttcgccaacgagggcttcctcggctaccagtacgagtgccccggcctgatcgaggtcccaccgagcaggaccccagca |
| | agtcctactgggtgatgttcatctccatcaaccccggcgccccggccggcggctccttcaaccagtacttcgtcggcagcttcaacggcac |
| | ccacttcgaggccttcgacaaccagtcccgcgtggtggacttcggcaaggactactacgccctgcagaccttcttcaacaccgacccgac |
| | ctacgggagcgccctgggcatcgcgtgggcctccaactgggagtactccgccttcgtgcccaccaacccctggcgctcctccatgtccctc |
| | gtgcgcaagttctccctcaacaccgagtaccaggccaacccggagacggagctgatcaacctgaaggccgagccgatcctgaacatc |
| | agcaacgccggcccctggagccggttcgccaccaacaccacgttgacgaaggccaacagctacaacgtcgacctgtccaacagcacc |
| | ggcaccctggagttcgagctggtgtacgccgtcaacaccacccagacgatctccaagtccgtgttcgcggacctctccctctggttcaagg |
| | gcctggaggaccccgaggagtacctccgcatgggcttcgaggtgtccgcgtcctccttcttcctggaccgcgggaacagcaaggtgaag |
| | ttcgtgaaggagaacccctacttcaccaaccgcatgagcgtgaacaaccagcccttcaagagcgagaacgacctgtcctactacaagg |
| | tgtacggcttgctggaccagaacatcctggagctgtacttcaacgacggcgacgtcgtgtccaccaacacctacttcatgaccaccggga |
| | acgccctgggctccgtgaacatgacgacgggggtggacaacctgttctacatcgacaagttccaggtgcgcgaggtcaagtgactTA |
| | AGACGCCCGCGCGGCGCACCTGACCTGTTCTCTCGAGGGCGCCTGTTCTGCCTTGCGA |
| | AACAAGCCCCTGGAGCATGCGTGCATGATCGTCTCTGGCGCCCCGCCGCGCGGTTTGT |
| | CGCCCTCGCGGGCGCCGCGGCCGCGGGGGCGCATTGAAATTGTTGCAAACCCCACCT |
| | GACAGATTGAGGGCCCAGGCAGGAAGGCGTTGAGATGGAGGTACAGGAGTCAAGTA |
| | ACTGAAAGTTTTTATGATAACTAACAACAAAGGGTCGTTTCTGGCCAGCGAATGACA |
| | AGAACAAGATTCCACATTTCCGTGTAGAGGCTTGCCATCGAATGTGAGCGGGCGGGC |
| | CGCGGACCCGACAAAACCCTTACGACGTGGTAAGAAAAACGTGGCGGGCACTGTCCC |
| | TGTAGCCTGAAGACCAGCAGGAGACGATCGGAAGCATCACAGCACAACCGGtgcagcag |
| | cagctcggatagtatcgacacactctggacgctggtcgtgtgatggactgttgccgccacacttgctgccttgacctgtgaatatccctgcc |
| | gcttttatcaaacagcctcagtgtgtttgatcttgtgtgtacgcgcttttgcgagttgctagctgcttgtgctatttgcgaataccaccccc |
| | agcatccccttccctcgtttcatatcgcttgcatcccaaccgcaacttatctacgctgtcctgctatccctcagcgctgctcctgctcctgc |
| | tcactgcccctcgcacagccttggtttgggctccgcctgtattctcctggtactgcaacctgtaaaccagcactgcaatgctgatgcacggg |
| | 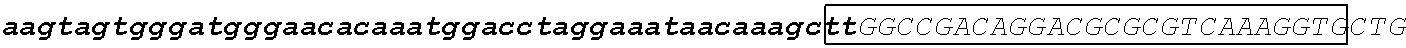 |
| | 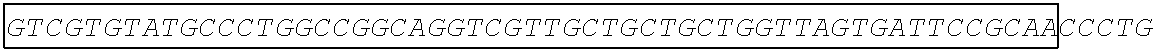 |
| | 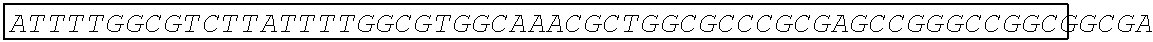 |
| | 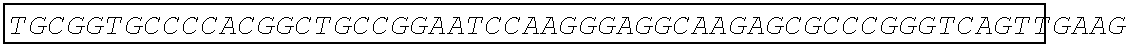 |
| | 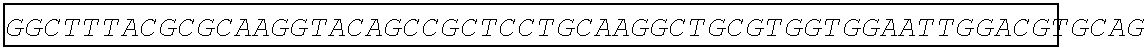 |
| | 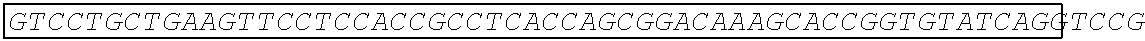 |
| | 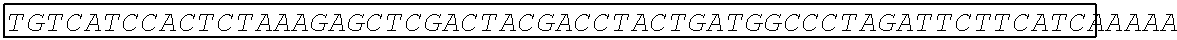 |
| | 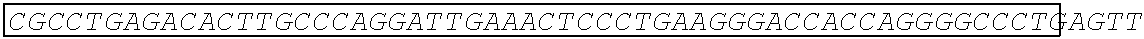 |
| | 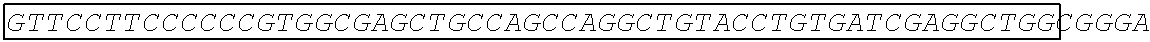 |
| | 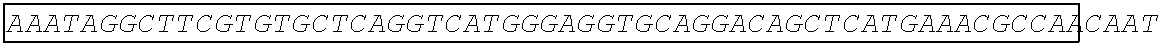 |
| | 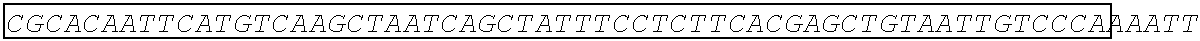 |
| | 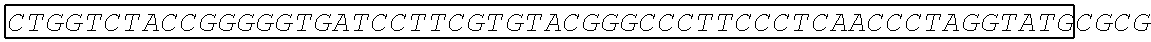 |
| | 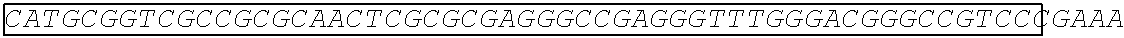 |
| | 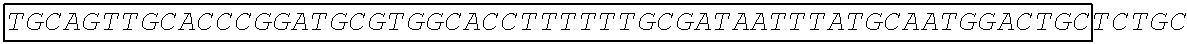 |
| | 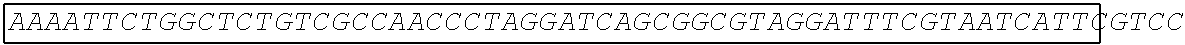 |
| | 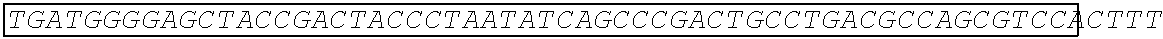 |
| | 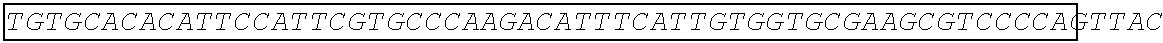 |
| | 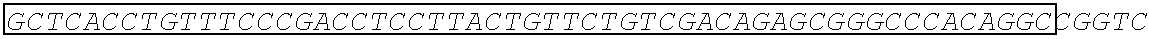 |
| | 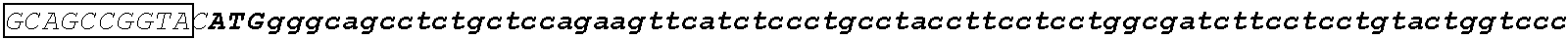 |
| | tcccgatctgcgtgctggtcggcaccctgaagctcctggatttcaccttcccgcggacggatatgtacaactggtgccagtttatgaccaag |
| | ctgttccggatcaagatcaagaagtacggccctaacggcctgcgcaaggacacgggccgccacatcctctacctgtgcaaccaccgctc |
| | gtggaccgactttttcctggacgcttacctgacggagggcaacggctcgctcatgtcgcgctggctggtgttcgcggtcttcccggtcttta |
| | tggtggcggtgatcgtgctgcgcggagtcatcctgttcaagcggggcacggtgctcgacaaggagaagttcaacgagtggctggactccaa |
| | ggtgtcctcttcgccgatccccggcatgctcgtctaccccgaggggcaccgctcgctcgctcataagagcctcccgctcaagcgcgggat |
| | gctgtactacgcgcactcccgcaagtttcccgtgcagatcattatcagccgcaacaaggaggaggtgctgtccgagaagacgatgaag |
| | gcccactttggcgccacgatcgtcacgggcttctccgagctgatcgagtcggagggcaaggacatcaaggagttcctgagcgaggtcca |
| | gacggcctgggacaagatgtgggactctgtgtactccgccgacgctgccgcgctccccgagctggagcatacggacgtccccaagttcg |
| | actactcgtttagcatgcgcgtgatgcagctcctggtcgtgtccggcaccgtggtcgtgttcctcggcacgatggcggccatggtcaagtgc |
| | gtgtgcgtgtgcctcgccgctacgggcgacctcggctgcaaggtgctcgtcagctcgctcgtgggctggtttctggtgtccatcctgaacag |
| | ccgagcgccctccggcaagTGAcacgcccgcgcggcgcacctgacctgttctctcgagggcgcctgttctgccttgcgaaacaagcccc |
| | tggagcatgcgtgcatgatcgtctctggcgccccgccgcgcggtttgtcgccctcgcgggcgccgcggccgcgggggcgcattgaaattgt |
| | tgcaaaccccacctgacagattgagggcccaggcaggaaggcgttgagatggaggtacaggagtcaagtaactgaaagtttttatgataact |
| | aacaacaaagggtcgtttctggccagcgaatgacaagaacaagattccacatttccgtgtagaggcttgccatcgaatgtgagcgggcgggc |
| | cgcggacccgacaaaacccttacgacgtggtaagaaaaacgtggcgggcactgtccctgtagcctgaagaccagcaggagacgatcgga |
| | agcatcacagcacaaccggtatcgatagcgtctgcgtgttgggagctggagtcgtgggcttgacgacggcgctgcagctcttgcaggat |
| | gtgcctggcgtgcgcgttcacgtcgtggctgagaaatatggcgacgaaacgttgacggctggggccggcgggctgtggatgccatac |
| | gcattgggtacgcggccattggatgggattggtaggcttatggagggataatagagtttttgtcggatccaacgcatgttgatgcagta |
| | gcccggtgtgctgaaagtatggaaggatggtgcattggctattcacatgcgctgcccaccccttttcgcaggaaatgtgccggcatcgt |
| | tggtgcaccgatggggaaaatcgacgttcgaccactacatgaaaatgtatacctctgaagatgcagcaactgcgggtgcgaaacgg |
| | ataacggtttggtaggtgtatgtcgcagcatgtgctggattttgcgggcaactccccctgccacggcccattgcaggtgtcatgttgact |
| | ggaggatacgaactttcggccgtcaaattcccagaggaggaccccctctgggccgacattgtgcccactgctcttccgcttgctgtttc |
| | ctgtgtgaaattgt |

The construct can be written as 5′DAO1b::CrTUB2:ScSUC2:PmPGH:CvNR:PmAMT03:DsLPAAT2:PmPGH::3′DAO1b. Proceeding in the 5′ to 3′ direction bold, lowercase sequences represent genomic DNA from CHK22 that permit targeted integration at the DAO1b locus via homologous recombination, the C. rhinanthin β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text that enables amplification of the DsLPAAT2 gene, followed by a linker, indicated by lowercase, bold italics, in which contains a C. vulgaris nitrate reductase 3′-UTR. The P. moriformis PmAMT03 promoter, indicated by boxed italicized text, drives the expression of the DsLPAAT2. The initiator ATG and terminator TGA codons of the DsLPAAT2 are indicated by uppercase, bold italics, while the remainder of the gene is indicated by bold italics. The P. moriformis PmPGH 3′-UTR is indicated by lowercase underlined text followed by the CHK22 DAO1b genomic region indicated by bold, lowercase text.

Table 34 shows the fatty acid/TAG profiles of primary transformants of CHK22 transformed with pCHK971. The P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 34
Screen of primary transformants of CHK22 with pCHK971.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK971- | 0.06 | 1.56 | 21.15 | 3.35 | 56.64 | 14.85 | 0.63 | 0.23 | 0.11 | 2.16 |
| 1 | | | | | | | | | | |
| pCHK971- | 0.06 | 1.58 | 21.67 | 3.56 | 56.73 | 14.04 | 0.60 | 0.22 | 0.08 | 2.35 |
| 2 | | | | | | | | | | |
| pCHK971- | 0.06 | 1.58 | 20.50 | 3.36 | 56.76 | 15.24 | 0.67 | 0.24 | 0.08 | 2.11 |
| 3 | | | | | | | | | | |
| pCHK971- | 0.05 | 1.51 | 20.65 | 3.44 | 57.15 | 14.86 | 0.57 | 0.24 | 0.10 | 2.20 |
| 4 | | | | | | | | | | |
| pCHK971- | 0.04 | 1.44 | 24.31 | 3.15 | 59.43 | 9.30 | 0.49 | 0.30 | 0.09 | 1.57 |
| 5 | | | | | | | | | | |
| pCHK971- | 0.06 | 1.52 | 19.88 | 3.23 | 56.45 | 16.58 | 0.60 | 0.23 | 0.11 | 2.24 |
| 6 | | | | | | | | | | |
| CHK22 | 0.04 | 1.56 | 26.48 | 3.06 | 58.93 | 7.59 | 0.47 | 0.27 | 0.07 | 1.56 |
| Control | | | | | | | | | | |

### Example 17: Expressing Scenedesmus sp. LPAAT2 (SceLPAAT2) in P. moriformis Strain CHK22

SceLPAAT2 was introduced into a P. moriformis strain CHK22. The expression construct pCHK973 contained 5′ and 3′ homology arms to permit targeted integration of SceLPAAT2 into the genome and is shown in Table 35.

TABLE 35
Integrative sequences for the transformation of P. moriformis with pCHK973 encoding SceLPAAT2.

| SEQ ID | |
|---|---|
| NO: | Nucleotide Sequence |
| 29 | gctcttccgcttgcccgcaccctcgttgatctgggagccctgcgcagccccttaaatcatctcagtcaggtttctatgttcaactgagcct |
| | aaagggctttcgtcacgcgcacgagcacacgtatatcggctacgcagtctctcagaagcggtagaacagttcgcaagccctcgtcgg |
| | tcgaaaacttgcgccagtactattgaattaaattaaatgatcgaatgagacgcgaaacttttgcagaatgccactgagtttgcccaga |
| | gaatgggagtggcgccattcaccatccgcctgtgcacggcctgattcgccgagacgatgaatggcgagaccagggagcggcttgcg |
| | agccccgagccggtagcaggaataacggtcgacaatcttcctgtccaattactggcaaccattagaaagagccggagcgcgttgaa |
| | attctgcaatcgagtaatttttcgatgcggcgggcctgctgaaccctaaggctccggcctatgtttaaggcgatccaagatgcacgcgg |
| | ccccaggcacgtgtctcaagcacaaaccccagccttagtttcgagactttgggagatagcggcagatatctagtttggcattttgtatat |
| | taattaagtctcgcaatggagcgctctgatgcggtgcagcgtcggctgcagcacctggcagtggcgctggggtcgccctatcgctcgg |
| | 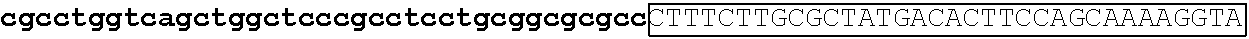 |
| | 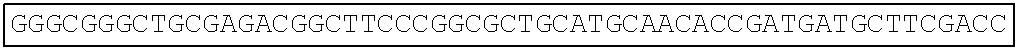 |
| | 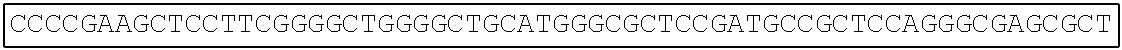 |
| | 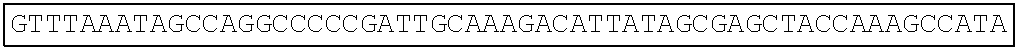 |
| | 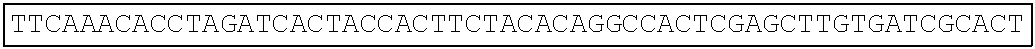 |
| | 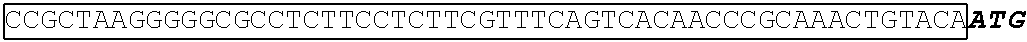 |
| | ctgctgcaggccttcctgttcctgctggccggcttcgccgccaagatcagcgcctccatgacgaacgagacgtccgaccgccccctggtg |
| | cacttcacccccaacaagggctggatgaacgaccccaacggcctgtggtacgacgagaaggacgccaagtggcacctgtacttccagt |
| | acaacccgaacgacaccgtctgggggacgcccttgttctggggccacgccacgtccgacgacctgaccaactgggaggaccagccca |
| | tcgccatcgccccgaagcgcaacgactccggcgccttctccggctccatggtggtggactacaacaacacctccggcttcttcaacgaca |
| | ccatcgacccgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctggtacgagc |
| | cctcccagaagtggatcatgaccgcggccaagtcccaggactacaagatcgagatctactcctccgacgacctgaagtcctggaagctg |
| | gagtccgcgttcgccaacgagggcttcctcggctaccagtacgagtgccccggcctgatcgaggtccccaccgagcaggaccccagca |
| | agtcctactgggtgatgttcatctccatcaaccccggcgccccggccggcggctccttcaaccagtacttcgtcggcagcttcaacggcac |
| | ccacttcgaggccttcgacaaccagtcccgcgtggtggacttcggcaaggactactacgccctgcagaccttcttcaacaccgacccgac |
| | ctacgggagcgccctgggcatcgcgtgggcctccaactgggagtactccgccttcgtgcccaccaacccctggcgctcctccatgtccctc |
| | gtgcgcaagttctccctcaacaccgagtaccaggccaacccggagacggagctgatcaacctgaaggccgagccgatcctgaacatc |
| | agcaacgccggcccctggagccggttcgccaccaacaccacgttgacgaaggccaacagctacaacgtcgacctgtccaacagcacc |
| | ggcaccctggagttcgagctggtgtacgccgtcaacaccacccagacgatctccaagtccgtgttcgcggacctctccctctggttcaagg |
| | gcctggaggaccccgaggagtacctccgcatgggcttcgaggtgtccgcgtcctccttcttcctggaccgcgggaacagcaaggtgaag |
| | ttcgtgaaggagaacccctacttcaccaaccgcatgagcgtgaacaaccagcccttcaagagcgagaacgacctgtcctactacaagg |
| | tgtacggcttgctggaccagaacatcctggagctgtacttcaacgacggcgacgtcgtgtccaccaacacctacttcatgaccaccggga |
| | acgccctgggctccgtgaacatgacgacgggggtggacaacctgttctacatcgacaagttccaggtgcgcgaggtcaagtgactTA |
| | AGACGCCCGCGCGGCGCACCTGACCTGTTCTCTCGAGGGCGCCTGTTCTGCCTTGCGA |
| | AACAAGCCCCTGGAGCATGCGTGCATGATCGTCTCTGGCGCCCCGCCGCGCGGTTTGT |
| | CGCCCTCGCGGGCGCCGCGGCCGCGGGGGCGCATTGAAATTGTTGCAAACCCCACCT |
| | GACAGATTGAGGGCCCAGGCAGGAAGGCGTTGAGATGGAGGTACAGGAGTCAAGTA |
| | ACTGAAAGTTTTTATGATAACTAACAACAAAGGGTCGTTTCTGGCCAGCGAATGACA |
| | AGAACAAGATTCCACATTTCCGTGTAGAGGCTTGCCATCGAATGTGAGCGGGCGGGC |
| | CGCGGACCCGACAAAACCCTTACGACGTGGTAAGAAAAACGTGGCGGGCACTGTCCC |
| | TGTAGCCTGAAGACCAGCAGGAGACGATCGGAAGCATCACAGCACAACCGGtgcagcag |
| | cagctcggatagtatcgacacactctggacgctggtcgtgtgatggactgttgccgccacacttgctgccttgacctgtgaatatccctgcc |
| | gcttttatcaaacagcctcagtgtgtttgatcttgtgtgtacgcgcttttgcgagttgctagctgcttgtgctatttgcgaataccaccccc |
| | agcatccccttccctcgtttcatatcgcttgcatcccaaccgcaacttatctacgctgtcctgctatccctcagcgctgctcctgctcctgc |
| | tcactgcccctcgcacagccttggtttgggctccgcctgtattctcctggtactgcaacctgtaaaccagcactgcaatgctgatgcacggg |
| | 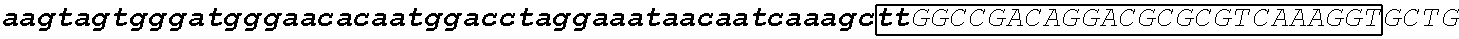 |
| | 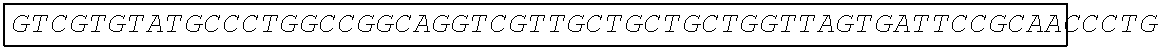 |
| | 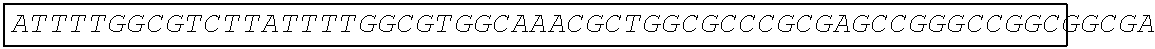 |
| | 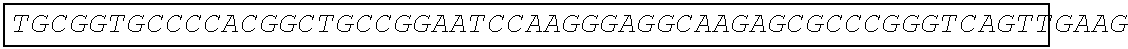 |
| | 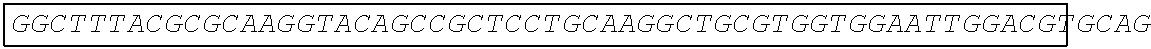 |
| | 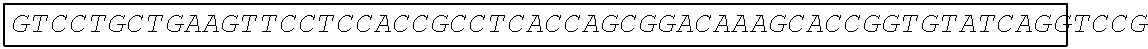 |
| | 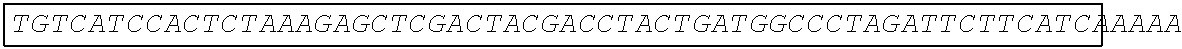 |
| | 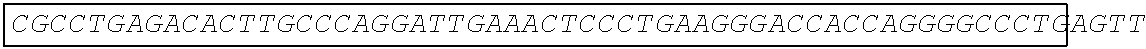 |
| | 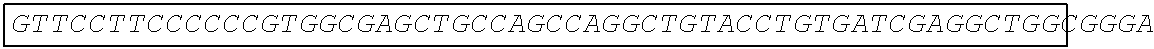 |
| | 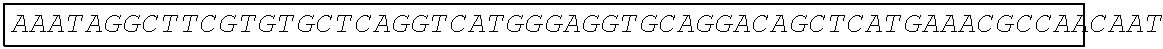 |
| | 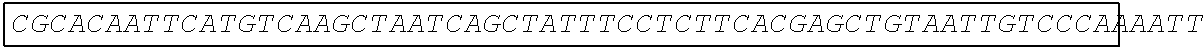 |
| | 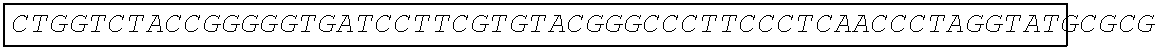 |
| | 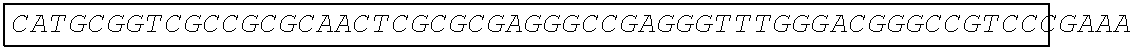 |
| | 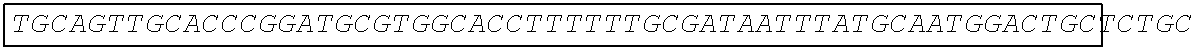 |
| | 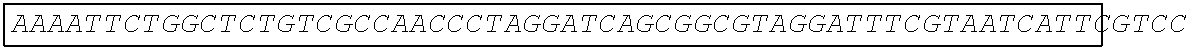 |
| | 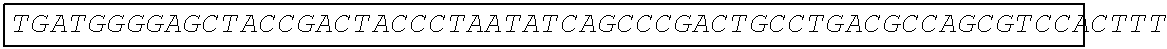 |
| | 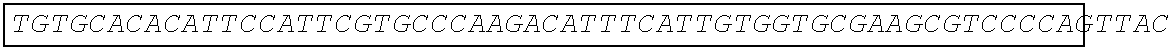 |
| | 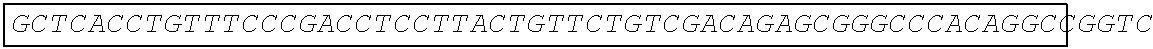 |
| | 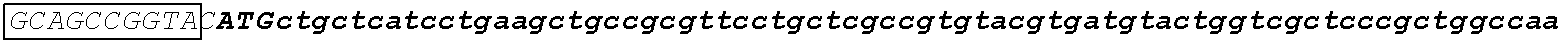 |
| | cctgctcctcttcatcaaggcttggaaggtcgtgggcaaccgaaacgacatgtatcggtggtgccagttcctcgtccgcgcctgtgggatc |
| | aaggtgcggaagtgcggcaagcaggaactctaccgtgggggcgcgtgcctgtacctggccaaccaccgatcgtgggccgacttctttgt |
| | cgacgcctacctgacggagggccggggccaactcatgtcccgcgccgcggtgatcttcgtctttcccgtcgtgatcctgcccctgacgctg |
| | gtgcgcgggtgcatttgctttcgccgggacggcgtgaaggacaaggagaagttcaaccgctggatcgacgcttgcatcggcgcctcgcc |
| | cgtccccggcatctgcctgttccccgagggccaccgctcgcgcctcccccattccctcccgctgaagcgtggcatgctgaactacgcgtac |
| | agccgcaagatgccagtgcagatcgtcatctctgcgcataaggagctggccatggacgaaaagagcttccacgtccgcttcggcgcca |
| | cctgcgtgaccgggtacggccccctgatgcgaaccgcggactacgcctccttcgacgagttctacaccgccctccagggcgagtggcag |
| | aagctctgggtggaggtcttctccgccgacccggcggccctcccggagctggtcatcagcgccccggcgggcttcacctaccccctgcaa |
| | ctgaagctgtcccagctcctgctcaccctcctggagctggcgctgatggcttggggcctcgcgtggtccgtccgtgcttggggcggggtgg |
| | cggcccgcctggcgggcaccctccaactgggcgtctcgggcctccaagtcctccagtacctgcccctggtctggctggtggtcagcctgg |
| | cgtcgtgctacgacagcacgctgccgcagcccgtcctggcggccgacggcacgccgctcgtcggctcctctcaagccacccgcgactcc |
| | aagaaggctctgTGAcacgcccgcgcggcgcacctgacctgttctctcgagggcgcctgttctgccttgcgaaacaagcccctggagcat |
| | gcgtgcatgatcgtctctggcgccccgccgcgcggtttgtcgccctcgcgggcgccgcggccgcgggggcgcattgaaattgttgcaaacc |
| | ccacctgacagattgagggcccaggcaggaaggcgttgagatggaggtacaggagtcaagtaactgaaagtttttatgataactaacaacaa |
| | agggtcgtttctggccagcgaatgacaagaacaagattccacatttccgtgtagaggcttgccatcgaatgtgagcgggcgggccgcggacc |
| | cgacaaaacccttacgacgtggtaagaaaaacgtggcgggcactgtccctgtagcctgaagaccagcaggagacgatcggaagcatcaca |
| | gcacaaccggtatcgatagcgtctgcgtgttgggagctggagtcgtgggcttgacgacggcgctgcagctcttgcaggatgtgcctgg |
| | cgtgcgcgttcacgtcgtggctgagaaatatggcgacgaaacgttgacggctggggccggcgggctgtggatgccatacgcattggg |
| | tacgcggccattggatgggattggtaggcttatggagggataatagagtttttgtcggatccaacgcatgttgatgcagtagcccggtg |
| | tgctgaaagtatggaaggatggtgcattggctattcacatgcgctgcccaccccttttcgcaggaaatgtgccggcatcgttggtgcac |
| | cgatggggaaaatcgacgttcgaccactacatgaaaatgtatacctctgaagatgcagcaactgcgggtgcgaaacggataacggt |
| | ttggtaggtgtatgtcgcagcatgtgctggattttgcgggcaactccccctgccacggcccattgcaggtgtcatgttgactggaggat |
| | acgaactttcggccgtcaaattcccagaggaggaccccctctgggccgacattgtgcccactgctcttccgcttgctgtttcctgtgtga |
| | aattgt |

The construct can be written as 5′DAO1b::CrTUB2:ScSUC2:PmPGH:CvNR:PmAMT03:SceLPAAT2:PmPGH::3′DAO1b. Proceeding in the 5′ to 3′ direction, bold, lowercase sequences represent genomic DNA from CHK22 that permit targeted integration at the DAO1b locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text that enables amplification of the SceLPAAT2 gene, followed by a linker, indicated by lowercase, bold italics, in which contains a C. vulgaris nitrate reductase 3′-UTR. The P. moriformis PmAMT03 promoter, indicated by boxed italicized text, drives the expression of the SceLPAAT2. The initiator ATG and terminator TGA codons of the SceLPAAT2 are indicated by uppercase, bold italics, while the remainder of the gene is indicated by bold italics. The P. moriformis PmPGH 3′-UTR is indicated by lowercase underlined text followed by the CHK22 DAO1b genomic region indicated by bold, lowercase text.

Table 36 shows the fatty acid/TAG profiles of primary transformants of CHK22 transformed with pCHK973. The P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 36
Screen of primary transformants of CHK22 with pCHK973.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK973- | 0.05 | 1.54 | 23.14 | 3.14 | 58.67 | 11.06 | 0.57 | 0.30 | 0.10 | 1.56 |
| 1 | | | | | | | | | | |
| pCHK973- | 0.06 | 1.51 | 21.14 | 3.51 | 55.80 | 15.58 | 0.63 | 0.24 | 0.08 | 1.64 |
| 2 | | | | | | | | | | |
| pCHK973- | 0.05 | 1.51 | 22.45 | 3.02 | 58.88 | 11.65 | 0.52 | 0.28 | 0.10 | 1.62 |
| 3 | | | | | | | | | | |
| pCHK973- | 0.07 | 1.65 | 19.94 | 3.49 | 54.37 | 17.96 | 0.68 | 0.26 | 0.12 | 1.59 |
| 4 | | | | | | | | | | |
| pCHK973- | 0.06 | 1.65 | 20.99 | 3.57 | 54.54 | 16.80 | 0.63 | 0.24 | 0.12 | 1.54 |
| 5 | | | | | | | | | | |
| pCHK973- | 0.06 | 1.56 | 21.96 | 3.40 | 55.61 | 15.04 | 0.59 | 0.24 | 0.11 | 1.64 |
| 6 | | | | | | | | | | |
| CHK22 | 0.04 | 1.56 | 26.48 | 3.06 | 58.93 | 7.59 | 0.47 | 0.27 | 0.07 | 1.56 |
| Control | | | | | | | | | | |

### Example 18: Expressing Micractinum conductrix LPAAT2 (McLPAAT2) in P. moriformis Strain CHK22

McLPAAT2 was introduced into a P. moriformis strain CHK22. The expression construct pCHK974 contained 5Y and 3Y homology arms to permit targeted integration of McLPAAT2 into the genome and is shown in Table 37.

TABLE 37
Integrative sequences for the transformation of P. moriformis with pCHK974 encoding McLPAAT2.

| SEQ ID | |
|---|---|
| NO: | Nucleotide Sequence |
| 30 | gctcttccgcttgcccgcaccctcgttgatctgggagccctgcgcagccccttaaatcatctcagtcaggtttctatgttcaactgagccta |
| | aagggctttcgtcacgcgcacgagcacacgtatatcggctacgcagtctctcagaagcggtagaacagttcgcaagccctcgtcggtc |
| | gaaaacttgcgccagtactattgaattaaattaaatgatcgaatgagacgcgaaacttttgcagaatgccactgagtttgcccagagaa |
| | tgggagtggcgccattcaccatccgcctgtgcacggcctgattcgccgagacgatgaatggcgagaccagggagcggcttgcgagcc |
| | ccgagccggtagcaggaataacggtcgacaatcttcctgtccaattactggcaaccattagaaagagccggagcgcgttgaaattctg |
| | caatcgagtaatttttcgatgcggcgggcctgctgaaccctaaggctccggcctatgtttaaggcgatccaagatgcacgcggccccag |
| | gcacgtgtctcaagcacaaaccccagccttagtttcgagactttgggagatagcggcagatatctagtttggcattttgtatattaattaa |
| | gtctcgcaatggagcgctctgatgcggtgcagcgtcggctgcagcacctggcagtggcgctggggtcgccctatcgctcggcgcctggt |
| | 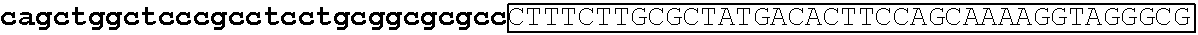 |
| | 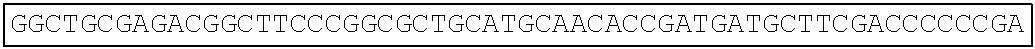 |
| | 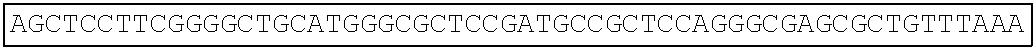 |
| | 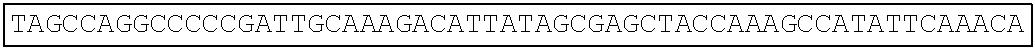 |
| | 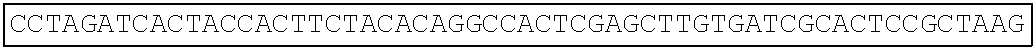 |
| | 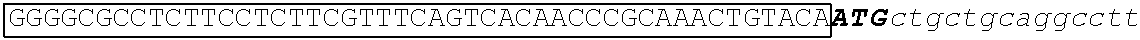 |
| | cctgttcctgctggccggcttcgccgccaagatcagcgcctccatgacgaacgagacgtccgaccgccccctggtgcacttcacccccaac |
| | aagggctggatgaacgaccccaacggcctgtggtacgacgagaaggacgccaagtggcacctgtacttccagtacaacccgaacgac |
| | accgtctgggggacgcccttgttctggggccacgccacgtccgacgacctgaccaactgggaggaccagcccatcgccatcgccccgaa |
| | gcgcaacgactccggcgccttctccggctccatggtggtggactacaacaacacctccggcttcttcaacgacaccatcgacccgcgcca |
| | gcgctgcgtggccatctggacctacaacaccccggagtccgaggagcagtacatctcctacagcctggacggcggctacaccttcaccga |
| | gtaccagaagaaccccgtgctggccgccaactccacccagttccgcgacccgaaggtcttctggtacgagccctcccagaagtggatcat |
| | gaccgcggccaagtcccaggactacaagatcgagatctactcctccgacgacctgaagtcctggaagctggagtccgcgttcgccaacg |
| | agggcttcctcggctaccagtacgagtgccccggcctgatcgaggtccccaccgagcaggaccccagcaagtcctactgggtgatgttcat |
| | ctccatcaaccccggcgccccggccggcggctccttcaaccagtacttcgtcggcagcttcaacggcacccacttcgaggccttcgacaac |
| | cagtcccgcgtggtggacttcggcaaggactactacgccctgcagaccttcttcaacaccgacccgacctacgggagcgccctgggcatc |
| | gcgtgggcctccaactgggagtactccgccttcgtgcccaccaacccctggcgctcctccatgtccctcgtgcgcaagttctccctcaacacc |
| | gagtaccaggccaacccggagacggagctgatcaacctgaaggccgagccgatcctgaacatcagcaacgccggcccctggagccgg |
| | ttcgccaccaacaccacgttgacgaaggccaacagctacaacgtcgacctgtccaacagcaccggcaccctggagttcgagctggtgta |
| | cgccgtcaacaccacccagacgatctccaagtccgtgttcgcggacctctccctctggttcaagggcctggaggaccccgaggagtacctc |
| | cgcatgggcttcgaggtgtccgcgtcctccttcttcctggaccgcgggaacagcaaggtgaagttcgtgaaggagaacccctacttcacca |
| | accgcatgagcgtgaacaaccagcccttcaagagcgagaacgacctgtcctactacaaggtgtacggcttgctggaccagaacatcctg |
| | gagctgtacttcaacgacggcgacgtcgtgtccaccaacacctacttcatgaccaccgggaacgccctgggctccgtgaacatgacgacg |
| | ggggtggacaacctgttctacatcgacaagttccaggtgcgcgaggtcaagtgactTAAGACGCCCGCGCGGCGCACC |
| | TGACCTGTTCTCTCGAGGGCGCCTGTTCTGCCTTGCGAAACAAGCCCCTGGAGCATGCG |
| | TGCATGATCGTCTCTGGCGCCCCGCCGCGCGGTTTGTCGCCCTCGCGGGCGCCGCGGCC |
| | GCGGGGGCGCATTGAAATTGTTGCAAACCCCACCTGACAGATTGAGGGCCCAGGCAG |
| | GAAGGCGTTGAGATGGAGGTACAGGAGTCAAGTAACTGAAAGTTTTTATGATAACTAA |
| | CAACAAAGGGTCGTTTCTGGCCAGCGAATGACAAGAACAAGATTCCACATTTCCGTGT |
| | AGAGGCTTGCCATCGAATGTGAGCGGGCGGGCCGCGGACCCGACAAAACCCTTACGA |
| | CGTGGTAAGAAAAACGTGGCGGGCACTGTCCCTGTAGCCTGAAGACCAGCAGGAGAC |
| | GATCGGAAGCATCACAGCACAACCGGtgcagcagcagctcggatagtatcgacacactctggacgctggtcgtgtgatggactgttgccgcc |
| | acacttgctgccttgacctgtgaatatccctgccgcttttatcaaacagcctcagtgtgtttgatcttgtgtgtacgcgcttttgcgagttg |
| | ctagctgcttgtgctatttgcgaataccacccccagcatccccttccctcgtttcatatcgcttgcatcccaaccgcaacttatctacgctg |
| | tcctgctatccctcagcgctgctcctgctcctgctcactgcccctcgcacagccttggtttgggctccgcctgtattctcctggtactgc |
| | 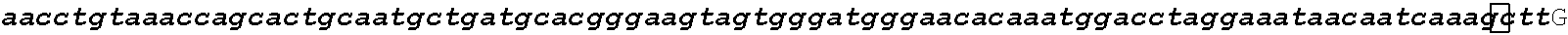 |
| | 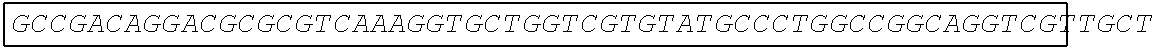 |
| | 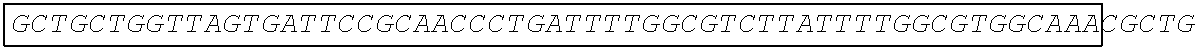 |
| | 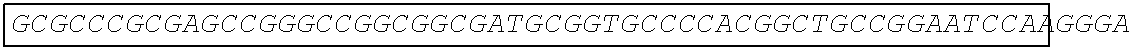 |
| | 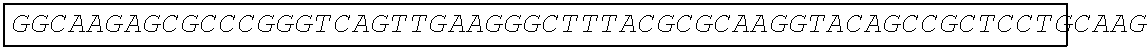 |
| | 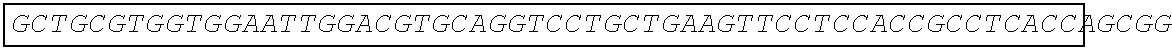 |
| | 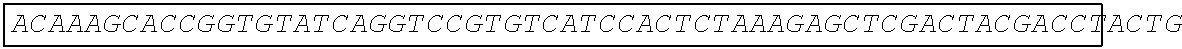 |
| | 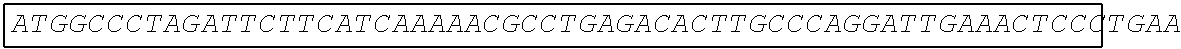 |
| | 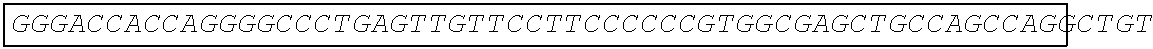 |
| | 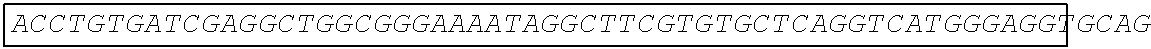 |
| | 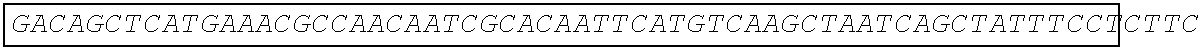 |
| | 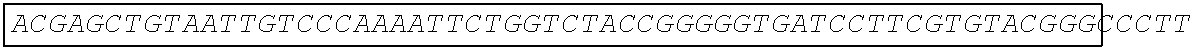 |
| | 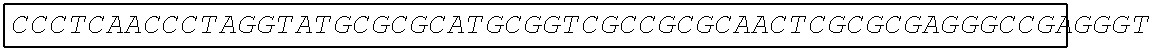 |
| | 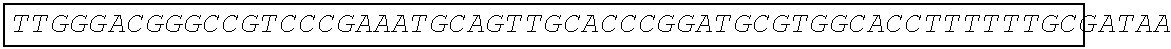 |
| | 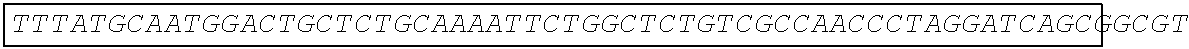 |
| | 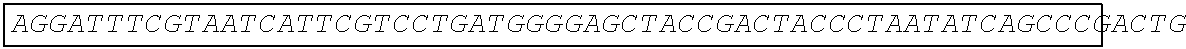 |
| | 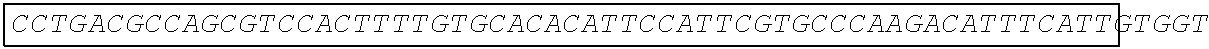 |
| | 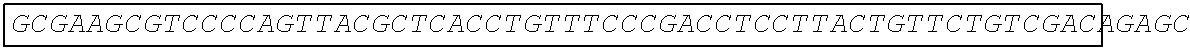 |
| | 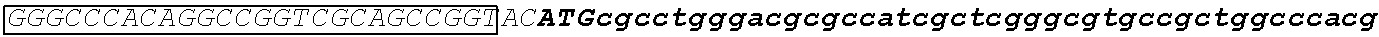 |
| | ttcctcggctcgatcctgctcctgtactggagcgtggtctgcgtggcgctcagctaccacttcaagtggctgaacctgcgcggccgacgcaac |
| | gatgtcctgggctggagcggaggcatgaagtggtgggtccgtgccaaggtcctgaaggtcggggaccgggaactctaccgcggcccttg |
| | cgtgtacctgttcaaccaccgctcctggggcgacttcatcgtcgaccagtacgccaccgagggccgttcgctgttcatgtctcggctgaccgt |
| | cctgttcgtctttccgacgtttatgggcgctctgaaggtgctgaagtcggtgatcacctttaagcgcggcgccattgccgacaaggagcgattt |
| | aacaactggattgatgagcagcgcgcggcctctccccagacgggcctctccgtctaccccgagggccatcgctctacggccccggtgtcc |
| | ctgcccctgaagcgcggcatgctgtactacgcgcactcgcggcgcctgcccgtccaggtcatcattggggccaacaaggaggcgattatc |
| | agcgagaagcactgcaccgcccgcctgaaccagaccgcggccgtggggtacagcgaccctatcatgaccgccgactaccccgactttg |
| | aggcgttctgggagcaggtgcaggaggcctggaacaagacgtgggcggaggtgtacggtgctgactgggagggactgccagaactcg |
| | ctccggggtccgcgggctggcaggaccccgacacgcactaccctctgtccgtgacgctgcccatggtcggggtggtcctggccaactttgc |
| | ggccttcgctggcgtctcctggctggccttccgcgcgctcagcgccctcttcgccctgttcggccctgcgcagcccctggcgctggcgctggtc |
| | gcggcttacctcgccctctccttctacctgtacagccgtccggtcgacgccctccagctccacgagcgcctgcgccgtgagtccaagcatcc |
| | gagccatctggcccgctccgctgcccacctggcgggcccgcgcggtgtggacgcgaacggcgctgccgctgccgaggcccgagtggcg |
| | gctctgcggcacgcgcggccgacgcatcgacccacgccctacctcgggcggcgacggccgctcgcggccccacccgctcgggctccgc |
| | cccggctgtcgctccgcgctccctccgcggctaccgccgtggcccgcggcatgctgTGAcacgcccgcgcggcgcacctgacctgttctc |
| | tcgagggcgcctgttctgccttgcgaaacaagcccctggagcatgcgtgcatgatcgtctctggcgccccgccgcgcggtttgtcgccctcgc |
| | gggcgccgcggccgcgggggcgcattgaaattgttgcaaaccccacctgacagattgagggcccaggcaggaaggcgttgagatggaggt |
| | acaggagtcaagtaactgaaagtttttatgataactaacaacaaagggtcgtttctggccagcgaatgacaagaacaagattccacatttccgtgt |
| | agaggcttgccatcgaatgtgagcgggcgggccgcggacccgacaaaacccttacgacgtggtaagaaaaacgtggcgggcactgtccctg |
| | tagcctgaagaccagcaggagacgatcggaagcatcacagcacaaccggtatcgatagcgtctgcgtgttgggagctggagtcgtgggct |
| | tgacgacggcgctgcagctcttgcaggatgtgcctggcgtgcgcgttcacgtcgtggctgagaaatatggcgacgaaacgttgacggc |
| | tggggccggcgggctgtggatgccatacgcattgggtacgcggccattggatgggattggtaggcttatggagggataatagagttttt |
| | gtcggatccaacgcatgttgatgcagtagcccggtgtgctgaaagtatggaaggatggtgcattggctattcacatgcgctgcccaccc |
| | cttttcgcaggaaatgtgccggcatcgttggtgcaccgatggggaaaatcgacgttcgaccactacatgaaaatgtatacctctgaaga |
| | tgcagcaactgcgggtgcgaaacggataacggtttggtaggtgtatgtcgcagcatgtgctggattttgcgggcaactccccctgccac |
| | ggcccattgcaggtgtcatgttgactggaggatacgaactttcggccgtcaaattcccagaggaggaccccctctgggccgacattgtg |
| | cccactgctcttccgcttgctgtttcctgtgtgaaattgt |

The construct can be written as 5′DAO1b::CrTUB2:ScSUC2:PmPGH:CvNR:PmAMT03:McLPAAT2:PmPGH::3′DAO1b. Proceeding in the 5′ to 3′ direction, bold, lowercase sequences represent genomic DNA from CHK22 that permit targeted integration at the DAO1b locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text that enables amplification of the McLPAAT2 gene, followed by a linker, indicated by lowercase, bold italics, in which contains a C. vulgaris nitrate reductase 3′-UTR. The P. moriformis PmAMT03 promoter, indicated by boxed italicized text, drives the expression of the McLPAAT2. The initiator ATG and terminator TGA codons of the McLPAAT2 are indicated by uppercase, bold italics, while the remainder of the gene is indicated by bold italics. The P. moriformis PmPGH 3′-UTR is indicated by lowercase underlined text followed by the CHK22 DAO1b genomic region indicated by bold, lowercase text.

Table 38 shows the fatty acid/TAG profiles of primary transformants of CHK22 transformed with pCHK974. The P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 38
Screen of primary transformants of CHK22 with pCHK974.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK974- | 0.04 | 1.51 | 23.69 | 3.15 | 59.18 | 10.11 | 0.51 | 0.32 | 0.10 | 1.54 |
| 1 | | | | | | | | | | |
| pCHK974- | 0.06 | 1.68 | 21.27 | 3.61 | 57.79 | 13.10 | 0.59 | 0.33 | 0.11 | 1.49 |
| 2 | | | | | | | | | | |
| pCHK974- | 0.05 | 1.58 | 22.76 | 3.41 | 58.49 | 11.19 | 0.55 | 0.31 | 0.07 | 1.52 |
| 3 | | | | | | | | | | |
| pCHK974- | 0.05 | 1.51 | 20.53 | 3.45 | 58.36 | 13.52 | 0.58 | 0.32 | 0.11 | 1.56 |
| 4 | | | | | | | | | | |
| pCHK974- | 0.05 | 1.60 | 22.41 | 3.12 | 58.05 | 12.12 | 0.54 | 0.28 | 0.10 | 1.55 |
| 5 | | | | | | | | | | |
| pCHK974- | 0.04 | 1.49 | 24.72 | 3.15 | 59.16 | 9.06 | 0.51 | 0.28 | 0.07 | 1.54 |
| 6 | | | | | | | | | | |
| CHK22 | 0.04 | 1.56 | 26.48 | 3.06 | 58.93 | 7.59 | 0.47 | 0.27 | 0.07 | 1.56 |
| Control | | | | | | | | | | |

### Example 19: Expressing Chlorella sorokiniana LPAAT2 (ChsoLPAAT2) in P. moriformis Strain CHK22

ChsoLPAAT2 was introduced into a P. moriformis strain CHK22. The expression construct pCHK975 contained 5Y and 3Y homology arms to permit targeted integration of ChsoLPAAT2 into the genome and is shown in Table 39.

TABLE 39
Integrative sequences for the transformation of P. moriformis with pCHK975 encoding ChsoLPAAT2.

| SEQ ID | |
|---|---|
| NO: | Nucleotide Sequence |
| 31 | gctcttccgcttgcccgcaccctcgttgatctgggagccctgcgcagccccttaaatcatctcagtcaggtttctatgttcaactgagcct |
| | aaagggctttcgtcacgcgcacgagcacacgtatatcggctacgcagtctctcagaagcggtagaacagttcgcaagccctcgtcgg |
| | tcgaaaacttgcgccagtactattgaattaaattaaatgatcgaatgagacgcgaaacttttgcagaatgccactgagtttgcccaga |
| | gaatgggagtggcgccattcaccatccgcctgtgcacggcctgattcgccgagacgatgaatggcgagaccagggagcggcttgcg |
| | agccccgagccggtagcaggaataacggtcgacaatcttcctgtccaattactggcaaccattagaaagagccggagcgcgttgaa |
| | attctgcaatcgagtaatttttcgatgcggcgggcctgctgaaccctaaggctccggcctatgtttaaggcgatccaagatgcacgcgg |
| | ccccaggcacgtgtctcaagcacaaaccccagccttagtttcgagactttgggagatagcggcagatatctagtttggcattttgtatat |
| | taattaagtctcgcaatggagcgctctgatgcggtgcagcgtcggctgcagcacctggcagtggcgctggggtcgccctatcgctcgg |
| | 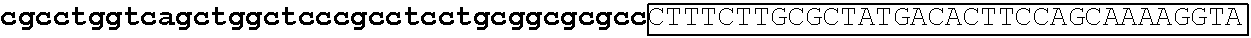 |
| | 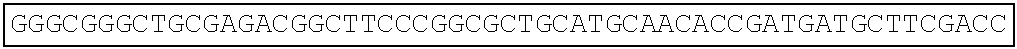 |
| | 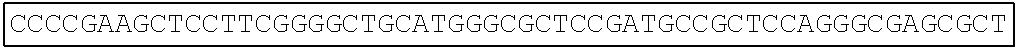 |
| | 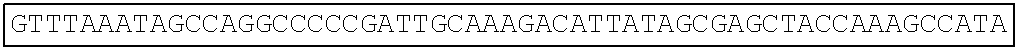 |
| | 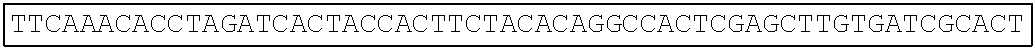 |
| | 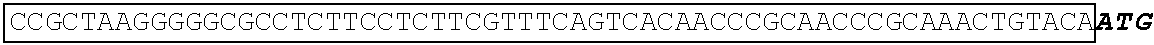 |
| | ctgctgcaggccttcctgttcctgctggccggcttcgccgccaagatcagcgcctccatgacgaacgagacgtccgaccgccccctggtg |
| | cacttcacccccaacaagggctggatgaacgaccccaacggcctgtggtacgacgagaaggacgccaagtggcacctgtacttccagt |
| | acaacccgaacgacaccgtctgggggacgcccttgttctggggccacgccacgtccgacgacctgaccaactgggaggaccagccca |
| | tcgccatcgccccgaagcgcaacgactccggcgccttctccggctccatggtggtggactacaacaacacctccggcttcttcaacgaca |
| | ccatcgacccgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctggtacgagc |
| | cctcccagaagtggatcatgaccgcggccaagtcccaggactacaagatcgagatctactcctccgacgacctgaagtcctggaagctg |
| | gagtccgcgttcgccaacgagggcttcctcggctaccagtacgagtgccccggcctgatcgaggtccccaccgagcaggaccccagca |
| | agtcctactgggtgatgttcatctccatcaaccccggcgccccggccggcggctccttcaaccagtacttcgtcggcagcttcaacggcac |
| | ccacttcgaggccttcgacaaccagtcccgcgtggtggacttcggcaaggactactacgccctgcagaccttcttcaacaccgacccgac |
| | ctacgggagcgccctgggcatcgcgtgggcctccaactgggagtactccgccttcgtgcccaccaacccctggcgctcctccatgtccctc |
| | gtgcgcaagttctccctcaacaccgagtaccaggccaacccggagacggagctgatcaacctgaaggccgagccgatcctgaacatc |
| | agcaacgccggcccctggagccggttcgccaccaacaccacgttgacgaaggccaacagctacaacgtcgacctgtccaacagcacc |
| | ggcaccctggagttcgagctggtgtacgccgtcaacaccacccagacgatctccaagtccgtgttcgcggacctctccctctggttcaagg |
| | gcctggaggaccccgaggagtacctccgcatgggcttcgaggtgtccgcgtcctccttcttcctggaccgcgggaacagcaaggtgaag |
| | ttcgtgaaggagaacccctacttcaccaaccgcatgagcgtgaacaaccagcccttcaagagcgagaacgacctgtcctactacaagg |
| | tgtacggcttgctggaccagaacatcctggagctgtacttcaacgacggcgacgtcgtgtccaccaacacctacttcatgaccaccggga |
| | acgccctgggctccgtgaacatgacgacgggggtggacaacctgttctacatcgacaagttccaggtgcgcgaggtcaagtgactTA |
| | AGACGCCCGCGCGGCGCACCTGACCTGTTCTCTCGAGGGCGCCTGTTCTGCCTTGCGA |
| | AACAAGCCCCTGGAGCATGCGTGCATGATCGTCTCTGGCGCCCCGCCGCGCGGTTTGT |
| | CGCCCTCGCGGGCGCCGCGGCCGCGGGGGCGCATTGAAATTGTTGCAAACCCCACCT |
| | GACAGATTGAGGGCCCAGGCAGGAAGGCGTTGAGATGGAGGTACAGGAGTCAAGTA |
| | ACTGAAAGTTTTTATGATAACTAACAACAAAGGGTCGTTTCTGGCCAGCGAATGACA |
| | AGAACAAGATTCCACATTTCCGTGTAGAGGCTTGCCATCGAATGTGAGCGGGCGGGC |
| | CGCGGACCCGACAAAACCCTTACGACGTGGTAAGAAAAACGTGGCGGGCACTGTCCC |
| | TGTAGCCTGAAGACCAGCAGGAGACGATCGGAAGCATCACAGCACAACCGGtgcagcag |
| | cagctcggatagtatcgacacactctggacgctggtcgtgtgatggactgttgccgccacacttgctgccttgacctgtgaatatccctgcc |
| | gcttttatcaaacagcctcagtgtgtttgatcttgtgtgtacgcgcttttgcgagttgctagctgcttgtgctatttgcgaataccaccccc |
| | agcatccccttccctcgtttcatatcgcttgcatcccaaccgcaacttatctacgctgtcctgctatccctcagcgctgctcctgctcctgc |
| | tcactgcccctcgcacagccttggtttgggctccgcctgtattctcctggtactgcaacctgtaaaccagcactgcaatgctgatgcacggg |
| | 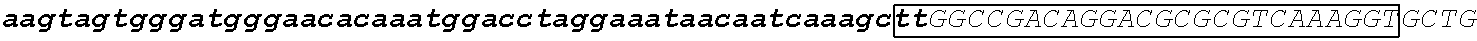 |
| | 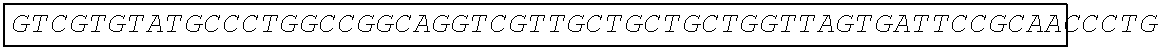 |
| | 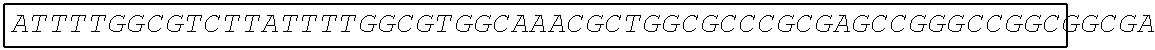 |
| | 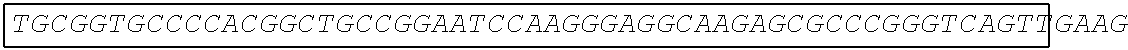 |
| | 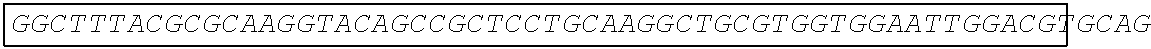 |
| | 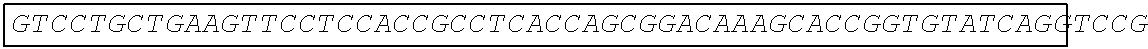 |
| | 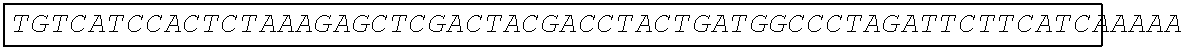 |
| | 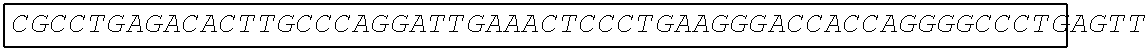 |
| | 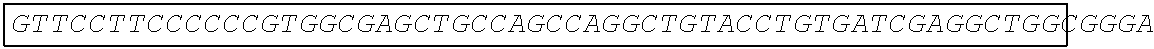 |
| | 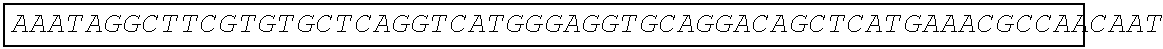 |
| | 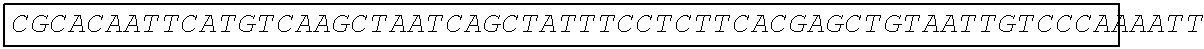 |
| | 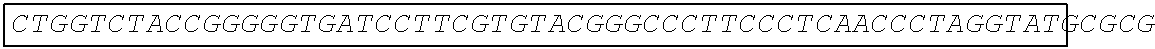 |
| | 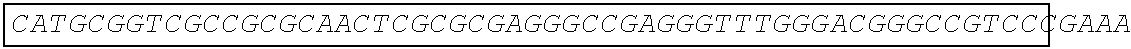 |
| | 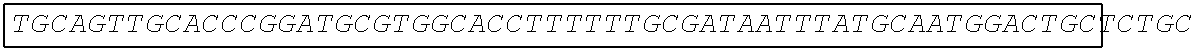 |
| | 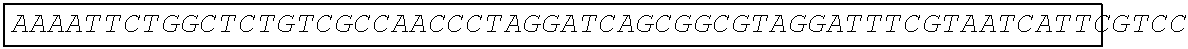 |
| | 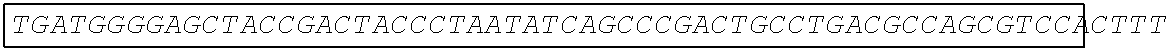 |
| | 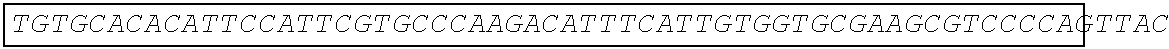 |
| | 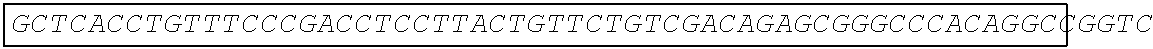 |
| | 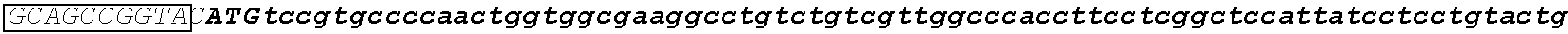 |
| | gtccgtggccttcatcaccctgagcggccacctgaagtggtggcgactccgtggtcgtcgcaacgacgtcctggcctggtcggctgggat |
| | gaagtggtgggtgcgatgccgagtcctgaaggtgggctctacggacctctaccgccagccgtgcgtgtacctgttcaaccaccgctcctg |
| | gggcgacttcatcgtggaccagtacgtgaccgaggggcggtctctgttcatgtcccggctggcggtcctgggggtgttccccacgttcatct |
| | acagcctcgtgatcctcaagtccattatcctcttcaagcggggctccatcgccgataagcagcggttcaacgcctggatcgacaagcagc |
| | gagagggctcgcctcagacgggcctggcggtgtacccagagggccatcggtcgaccctgggggagcccctcccgctgaagcgcggc |
| | atgctgcattacgcgtactcccgaaagctccccgtccagatcgtcatcggtgccaacaaggaggcgattatcgcggagaagcgctgcac |
| | cgctcgcctgggccaaaccgcggtcgtgggctactcggagccgattatcaccgccgattacccggacttcgaggcgttctgggcggagg |
| | tgcaagaggcgtgggacgctcagtggcaagctgtgtttggtgccgactggcagggcctgccggaactcgccgtccaggagcccgtgac |
| | gcactaccccctgtccatcaccctgccgatgctggggatgacctggctgaacctcggcctgtttgcgtggacctgctggctgacctttcgcg |
| | ccgtgcggtgggcctttgctctgtttggccccgcccagccgctggcgctggccgctgccgcggcctacctcgccctctccttctggctctactc |
| | ccagccagtcaacgcgctgaaggccatgcagacccagcgcggacaccccggcaagctcgctgagagcgaggctaacctggccgcg |
| | ctcgatggctcgggcgctcggcgcgacagcTGAcacgcccgcgcggcgcacctgacctgttctctcgagggcgcctgttctgccttgcg |
| | aaacaagcccctggagcatgcgtgcatgatcgtctctggcgccccgccgcgcggtttgtcgccctcgcgggcgccgcggccgcgggggc |
| | gcattgaaattgttgcaaaccccacctgacagattgagggcccaggcaggaaggcgttgagatggaggtacaggagtcaagtaactgaaag |
| | tttttatgataactaacaacaaagggtcgtttctggccagcgaatgacaagaacaagattccacatttccgtgtagaggcttgccatcgaatg |
| | tgagcgggcgggccgcggacccgacaaaacccttacgacgtggtaagaaaaacgtggcgggcactgtccctgtagcctgaagaccagcagga |
| | gacgatcggaagcatcacagcacaaccggtatcgatagcgtctcgctgttgggagctggagtcgtgggcttgacgacggcgctgcagc |
| | tcttgcaggatgtgcctggcgtgcgcgttcacgtcgtggctgagaaatatggcgacgaaacgttgacggctggggccggcgggctgt |
| | ggatgccatacgcattgggtacgcggccattggatgggattggtaggcttatggagggataatagagtttttgtcggatccaacgcat |
| | gttgatgcagtagcccggtgtgctgaaagtatggaaggatggtgcattggctattcacatgcgctgcccaccccttttcgcaggaaatg |
| | tgccggcatcgttggtgcaccgatggggaaaatcgacgttcgaccactacatgaaaatgtatacctctgaagatgcagcaactgcgg |
| | gtgcgaaacggataacggtttggtaggtgtatgtcgcagcatgtgctggattttgcgggcaactccccctgccacggcccattgcagg |
| | tgtcatgttgactggaggatacgaactttcggccgtcaaattcccagaggaggaccccctctgggccgacattgtgcccactgctcttc |
| | cgcttgctgtttcctgtgtgaaattgt |

The construct can be written as 5′DAO1b::CrTUB2:ScSUC2:PmPGH:CvNR:PmAMT03:ChsoLPAAT2:PmPGH::3′DAO1b. Proceeding in the 5′ to 3′ direction, bold, lowercase sequences represent genomic DNA from CHK22 that permit targeted integration at the DAO1b locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text that enables amplification of the ChsoLPAAT2 gene, followed by a linker, indicated by lowercase, bold italics, in which contains a Chlorella vulgaris nitrate reductase 3′-UTR. The P. moriformis PmAMT03 promoter, indicated by boxed italicized text, drives the expression of the ChsoLPAAT2. The initiator ATG and terminator TGA codons of the ChsoLPAAT2 are indicated by uppercase, bold italics, while the remainder of the gene is indicated by bold italics. The P. moriformis PmPGH 3′-UTR is indicated by lowercase underlined text followed by the CHK22 DAO1b genomic region indicated by bold, lowercase text.

Table 40 shows the fatty acid/TAG profiles of primary transformants of CHK22 transformed with pCHK975. The P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 40
Screen of primary transformants of CHK22 with pCHK975.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK975- | 0.07 | 0.03 | 20.71 | 3.44 | 57.62 | 14.19 | 0.59 | 0.26 | 0.12 | 1.53 |
| 1 | | | | | | | | | | |
| pCHK975- | 0.05 | 0.02 | 24.43 | 3.08 | 59.18 | 9.49 | 0.51 | 0.24 | 0.08 | 1.51 |
| 2 | | | | | | | | | | |
| pCHK975- | 0.06 | 0.02 | 20.71 | 3.4 | 57.82 | 14.16 | 0.57 | 0.24 | 0.11 | 1.49 |
| 3 | | | | | | | | | | |
| pCHK975- | 0.06 | 0.02 | 20.95 | 3.46 | 57.31 | 14.28 | 0.59 | 0.26 | 0.12 | 1.50 |
| 4 | | | | | | | | | | |
| pCHK975- | 0.06 | 0.02 | 20.92 | 3.5 | 57.12 | 14.39 | 0.57 | 0.25 | 0.11 | 1.51 |
| 5 | | | | | | | | | | |
| pCHK975- | 0.07 | 0.03 | 20.86 | 3.34 | 56.95 | 14.76 | 0.59 | 0.23 | 0.1 | 1.45 |
| 6 | | | | | | | | | | |
| CHK22 | 0.04 | 0.02 | 26.18 | 2.95 | 59.62 | 7.55 | 0.46 | 0.24 | 0.07 | 1.49 |
| Control | | | | | | | | | | |

### Example 20: Expressing Chlorella variabilis LPAAT2 (ChvaLPAAT2) in P. moriformis Strain CHK22

ChvaLPAAT2 was introduced into a P. moriformis strain CHK22. The expression construct pCHK976 contained 5′ and 3′ homology arms to permit targeted integration of ChvaLPAAT2 into the genome and is shown in Table 41.

TABLE 41
Integrative sequences for the transformation of P. moriformis with pCHK976 encoding ChvaLPAAT2.

| SEQ ID | |
|---|---|
| NO: | Nucleotide Sequence |
| 32 | gctcttccgcttgcccgcaccctcgttgatctgggagccctgcgcagccccttaaatcatctcagtcaggtttctatgttcaactgagcct |
| | aaagggctttcgtcacgcgcacgagcacacgtatatcggctacgcagtctctcagaagcggtagaacagttcgcaagccctcgtcgg |
| | tcgaaaacttgcgccagtactattgaattaaattaaatgatcgaatgagacgcgaaacttttgcagaatgccactgagtttgcccaga |
| | gaatgggagtggcgccattcaccatccgcctgtgcacggcctgattcgccgagacgatgaatggcgagaccagggagcggcttgcg |
| | agccccgagccggtagcaggaataacggtcgacaatcttcctgtccaattactggcaaccattagaaagagccggagcgcgttgaa |
| | attctgcaatcgagtaatttttcgatgcggcgggcctgctgaaccctaaggctccggcctatgtttaaggcgatccaagatgcacgcgg |
| | ccccaggcacgtgtctcaagcacaaaccccagccttagtttcgagactttgggagatagcggcagatatctagtttggcattttgtatat |
| | taattaagtctcgcaatggagcgctctgatgcggtgcagcgtcggctgcagcacctggcagtggcgctggggtcgccctatcgctcgg |
| | 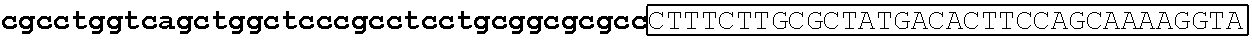 |
| | 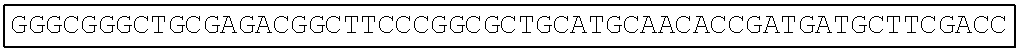 |
| | 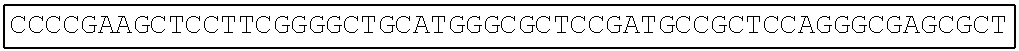 |
| | 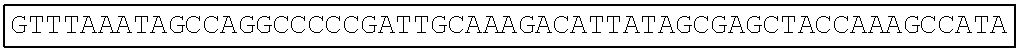 |
| | 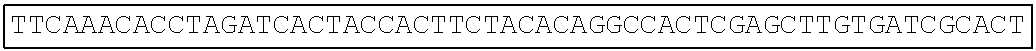 |
| | 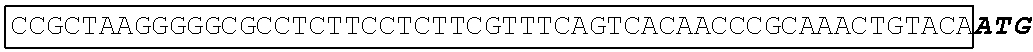 |
| | ctgctgcaggccttcctgttcctgctggccggcttcgccgccaagatcagcgcctccatgacgaacgagacgtccgaccgccccctggtg |
| | cacttcacccccaacaagggctggatgaacgaccccaacggcctgtggtacgacgagaaggacgccaagtggcacctgtacttccagt |
| | acaacccgaacgacaccgtctgggggacgcccttgttctggggccacgccacgtccgacgacctgaccaactgggaggaccagccca |
| | tcgccatcgccccgaagcgcaacgactccggcgccttctccggctccatggtggtggactacaacaacacctccggcttcttcaacgaca |
| | ccatcgacccgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctggtacgagc |
| | cctcccagaagtggatcatgaccgcggccaagtcccaggactacaagatcgagatctactcctccgacgacctgaagtcctggaagctg |
| | gagtccgcgttcgccaacgagggcttcctcggctaccagtacgagtgccccggcctgatcgaggtccccaccgagcaggaccccagca |
| | agtcctactgggtgatgttcatctccatcaaccccggcgccccggccggcggctccttcaaccagtacttcgtcggcagcttcaacggcac |
| | ccacttcgaggccttcgacaaccagtcccgcgtggtggacttcggcaaggactactacgccctgcagaccttcttcaacaccgacccgac |
| | ctacgggagcgccctgggcatcgcgtgggcctccaactgggagtactccgccttcgtgcccaccaacccctggcgctcctccatgtccctc |
| | gtgcgcaagttctccctcaacaccgagtaccaggccaacccggagacggagctgatcaacctgaaggccgagccgatcctgaacatc |
| | agcaacgccggcccctggagccggttcgccaccaacaccacgttgacgaaggccaacagctacaacgtcgacctgtccaacagcacc |
| | ggcaccctggagttcgagctggtgtacgccgtcaacaccacccagacgatctccaagtccgtgttcgcggacctctccctctggttcaagg |
| | gcctggaggaccccgaggagtacctccgcatgggcttcgaggtgtccgcgtcctccttcttcctggaccgcgggaacagcaaggtgaag |
| | ttcgtgaaggagaacccctacttcaccaaccgcatgagcgtgaacaaccagcccttcaagagcgagaacgacctgtcctactacaagg |
| | tgtacggcttgctggaccagaacatcctggagctgtacttcaacgacggcgacgtcgtgtccaccaacacctacttcatgaccaccggga |
| | acgccctgggctccgtgaacatgacgacgggggtggacaacctgttctacatcgacaagttccaggtgcgcgaggtcaagtgactTA |
| | AGACGCCCGCGCGGCGCACCTGACCTGTTCTCTCGAGGGCGCCTGTTCTGCCTTGCGA |
| | AACAAGCCCCTGGAGCATGCGTGCATGATCGTCTCTGGCGCCCCGCCGCGCGGTTTGT |
| | CGCCCTCGCGGGCGCCGCGGCCGCGGGGGCGCATTGAAATTGTTGCAAACCCCACCT |
| | GACAGATTGAGGGCCCAGGCAGGAAGGCGTTGAGATGGAGGTACAGGAGTCAAGTA |
| | ACTGAAAGTTTTTATGATAACTAACAACAAAGGGTCGTTTCTGGCCAGCGAATGACA |
| | AGAACAAGATTCCACATTTCCGTGTAGAGGCTTGCCATCGAATGTGAGCGGGCGGGC |
| | CGCGGACCCGACAAAACCCTTACGACGTGGTAAGAAAAACGTGGCGGGCACTGTCCC |
| | TGTAGCCTGAAGACCAGCAGGAGACGATCGGAAGCATCACAGCACAACCGGtgcagcag |
| | cagctcggatagtatcgacacactctggacgctggtcgtgtgatggactgttgccgccacacttgctgccttgacctgtgaatatccctgcc |
| | gcttttatcaaacagcctcagtgtgtttgatcttgtgtgtacgcgcttttgcgagttgctagctgcttgtgctatttgcgaataccaccccc |
| | agcatccccttccctcgtttcatatcgcttgcatcccaaccgcaacttatctacgctgtcctgctatccctcagcgctgctcctgctcctgc |
| | tcactgcccctcgcacagccttggtttgggctccgcctgtattctcctggtactgcaacctgtaaaccagcactgcaatgctgatgcacggg |
| | 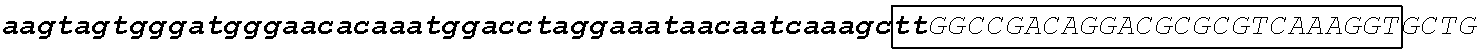 |
| | 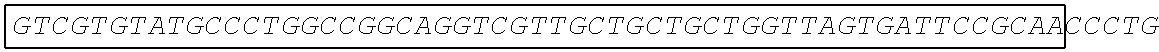 |
| | 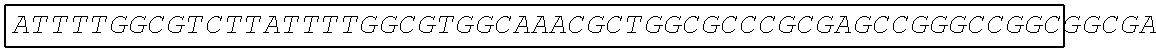 |
| | 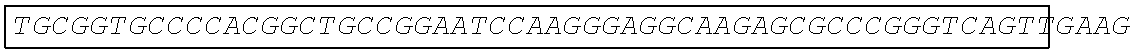 |
| | 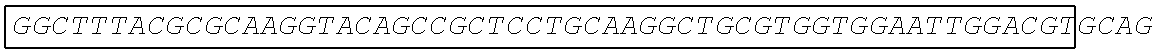 |
| | 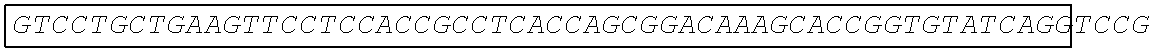 |
| | 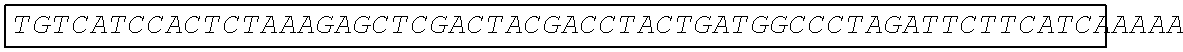 |
| | 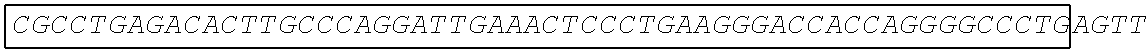 |
| | 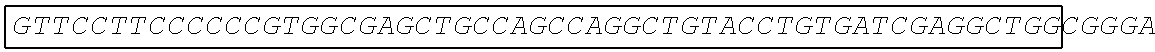 |
| | 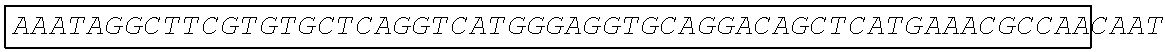 |
| | 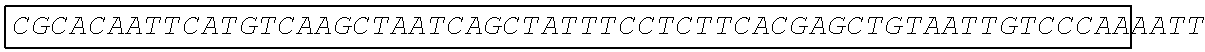 |
| | 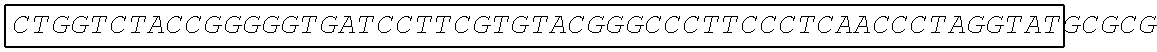 |
| | 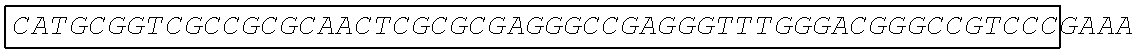 |
| | 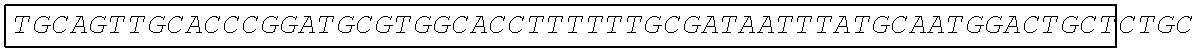 |
| | 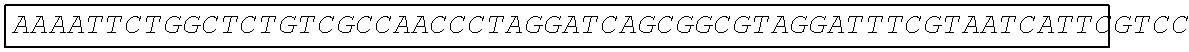 |
| | 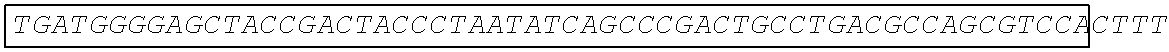 |
| | 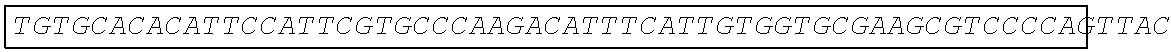 |
| | 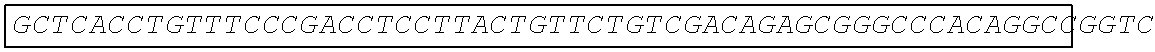 |
| | 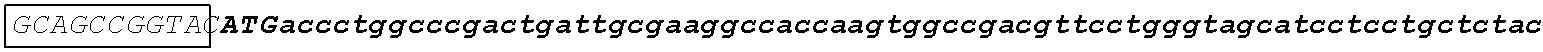 |
| | tggtccgtggtcttcgtcgcgctctcctaccacattaagtggctgaagctgaagggtcggcgcaacgacgtgctgggctgggccgctggc |
| | atgaagtggtggattcgctgtcgcgtcctgaaggtcggcacccaagagctgtaccgcgggcagtgcgtctacctcttcaaccaccgctcct |
| | ggggcgacttcatcgtggaccaatacgtcaccgagggacgcagcctgttcatgagccgcctggccgtcctcggggtctttcccaccttcgt |
| | gtcggctctcgtggtcctcaagagcattatcctgttcaagcgcggctccgtggccgacaaggagaagttcaatcggtggatcgacgagca |
| | gcgcgcggccagcccacagccggggctgtcggtgtacccagagggccaccgctccaccctgggggagccgctgcccctgaagcgcg |
| | gcatgctgcactacgccttctcccgcaagctccctattgtgatcggcgctaacaaggaggcgattatctccgagaagcactgcacggccc |
| | ggctgaaccagacggcggtggtcggatactctgcgccgatcatgaccgccaactaccccagcttcgacgcgttctgggaggaggtgca |
| | ggaggcgtggaacgcccagtgggccgcggtcttcggggccgactgggacggcctgcccgagctggccccacaggagccggataccg |
| | agtacccggcctcgatcaccctcgggatgctgggtgtggtcgtggccaacatggccctgttctgtctcacctgctggctgagcttccggttct |
| | ggcgttggctgttcgccctgttcggacccgcgcagcccgtggccctggccgctgccgcgggctacctggccctggctttttggctgtacagc |
| | cgcccggtgaacgccctccaactgcacgcggagaagcgccgaccgggccaactggcccagtcggctaccaacgtcgccgcgctgga |
| | cggcaagaaggcgtccTGAcacgcccgcgcggcgcacctgacctgttctctcgagggcgcctgttctgccttgcgaaacaagcccctgg |
| | agcatgcgtgcatgatcgtctctggcgccccgccgcgcggtttgtcgccctcgcgggcgccgcggccgcgggggcgcattgaaattgttgc |
| | aaaccccacctgacagattgagggcccaggcaggaaggcgttgagatggaggtacaggagtcaagtaactgaaagtttttatgataactaac |
| | aacaaagggtcgtttctggccagcgaatgacaagaacaagattccacatttccgtgtagaggcttgccatcgaatgtgagcgggcgggccgc |
| | ggacccgacaaaacccttacgacgtggtaagaaaaacgtggcgggcactgtccctgtagcctgaagaccagcaggagacgatcggaagc |
| | atcacagcacaaccggtatcgatagcgtctgcgtgttgggagctggagtcgtgggcttgacgacggcgctgcagctcttgcaggatgtg |
| | cctggcgtgcgcgttcacgtcgtggctgagaaatatggcgacgaaacgttgacggctggggccggcgggctgtggatgccatacgc |
| | attgggtacgcggccattggatgggattggtaggcttatggagggataatagagtttttgtcggatccaacgcatgttgatgcagtagc |
| | ccggtgtgctgaaagtatggaaggatggtgcattggctattcacatgcgctgcccaccccttttcgcaggaaatgtgccggcatcgttg |
| | gtgcaccgatggggaaaatcgacgttcgaccactacatgaaaatgtatacctctgaagatgcagcaactgcgggtgcgaaacggat |
| | aacggtttggtaggtgtatgtcgcagcatgtgctggattttgcgggcaactccccctgccacggcccattgcaggtgtcatgttgactgg |
| | aggatacgaactttcggccgtcaaattcccagaggaggaccccctctgggccgacattgtgcccactgctcttccgcttgctgtttcctg |
| | tgtgaaattgt |

The construct can be written as 5′DAO1b::CrTUB2:ScSUC2:PmPGH:CvNR:PmAMT03:ChvaLPAAT2:PmPGH::3′DAO1b Proceeding in the 5′ to 3′ direction bold, lowercase sequences represent genomic DNA from CHK22 that permit targeted integration at the DAO1b locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text that enables amplification of the ChvaLPAAT2 gene, followed by a linker, indicated by lowercase, bold italics, in which contains a C. vulgaris nitrate reductase 3′-UTR. The P. moriformis PmAMT03 promoter, indicated by boxed italicized text, drives the expression of the ChvaLPAAT2. The initiator ATG and terminator TAG codons of the ChvaLPAAT2 are indicated by uppercase, bold italics, while the remainder of the gene is indicated by bold italics. The P. moriformis PmPGH 3′-UTR is indicated by lowercase underlined text followed by the CHK22 DAO1b genomic region indicated by bold, lowercase text.

Table 42 shows the fatty acid/TAG profiles of primary transformants of CHK22 transformed with pCHK976. The P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 42
Screen of primary transformants of CHK22 with pCHK976.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK976- | 0.05 | 0.02 | 25.29 | 3.1 | 60.41 | 7.57 | 0.45 | 0.26 | 0.08 | 1.56 |
| 1 | | | | | | | | | | |
| pCHK976- | 0.05 | 0.02 | 23.82 | 3.11 | 59.49 | 9.74 | 0.5 | 0.27 | 0.09 | 1.51 |
| 2 | | | | | | | | | | |
| pCHK976- | 0.05 | 0.02 | 23.74 | 3.03 | 60.23 | 9.12 | 0.48 | 0.28 | 0.09 | 1.51 |
| 3 | | | | | | | | | | |
| pCHK976- | 0.05 | 0.02 | 23.45 | 3.19 | 59.56 | 9.93 | 0.54 | 0.3 | 0.1 | 1.58 |
| 4 | | | | | | | | | | |
| pCHK976- | 0.04 | 0.02 | 24.7 | 3.06 | 60.13 | 8.36 | 0.51 | 0.26 | 0.08 | 1.56 |
| 5 | | | | | | | | | | |
| pCHK976- | 0.06 | 0.03 | 21.49 | 3.27 | 58.89 | 12.13 | 0.57 | 0.3 | 0.11 | 1.50 |
| 6 | | | | | | | | | | |
| CHK22 | 0.04 | 0.02 | 26.18 | 2.95 | 59.62 | 7.55 | 0.46 | 0.24 | 0.07 | 1.49 |
| Control | | | | | | | | | | |

### Example 21: Expressing Raphidocelis subcapitata LPAAT2 (RsLPAAT2) in P. moriformis Strain CHK22

RsLPAAT2 was introduced into a P. moriformis strain CHK22. The expression construct pCHK977 contained 5Y and 3Y homology arms to permit targeted integration of RsLPAAT2 into the genome and is shown in Table 43.

TABLE 44
Integrative sequences for the transformation of P. moriformis with pCHK977 encoding RsLPAAT2.

| SEQ ID | |
|---|---|
| NO: | Nucleotide Sequence |
| 33 | gctcttccgcttgcccgcaccctcgttgatctgggagccctgcgcagccccttaaatcatctcagtcaggtttctatgttcaactgagcct |
| | aaagggctttcgtcacgcgcacgagcacacgtatatcggctacgcagtctctcagaagcggtagaacagttcgcaagccctcgtcgg |
| | tcgaaaacttgcgccagtactattgaattaaattaaatgatcgaatgagacgcgaaacttttgcagaatgccactgagtttgcccaga |
| | gaatgggagtggcgccattcaccatccgcctgtgcacggcctgattcgccgagacgatgaatggcgagaccagggagcggcttgcg |
| | agccccgagccggtagcaggaataacggtcgacaatcttcctgtccaattactggcaaccattagaaagagccggagcgcgttgaa |
| | attctgcaatcgagtaatttttcgatgcggcgggcctgctgaaccctaaggctccggcctatgtttaaggcgatccaagatgcacgcgg |
| | ccccaggcacgtgtctcaagcacaaaccccagccttagtttcgagactttgggagatagcggcagatatctagtttggcattttgtatat |
| | taattaagtctcgcaatggagcgctctgatgcggtgcagcgtcggctgcagcacctggcagtggcgctggggtcgccctatcgctcgg |
| | 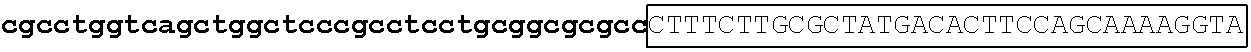 |
| | 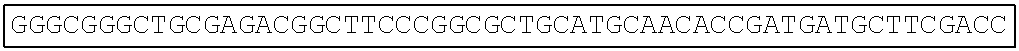 |
| | 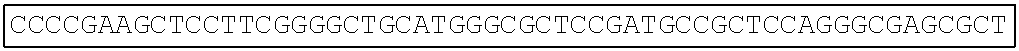 |
| | 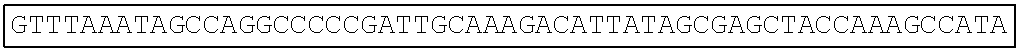 |
| | 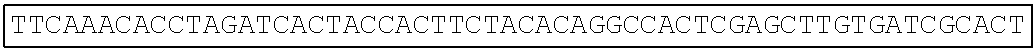 |
| | 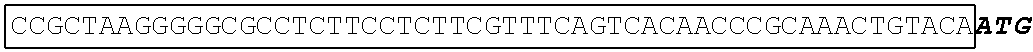 |
| | ctgctgcaggccttcctgttcctgctggccggcttcgccgccaagatcagcgcctccatgacgaacgagacgtccgaccgccccctggtg |
| | cacttcacccccaacaagggctggatgaacgaccccaacggcctgtggtacgacgagaaggacgccaagtggcacctgtacttccagt |
| | acaacccgaacgacaccgtctgggggacgcccttgttctggggccacgccacgtccgacgacctgaccaactgggaggaccagccca |
| | tcgccatcgccccgaagcgcaacgactccggcgccttctccggctccatggtggtggactacaacaacacctccggcttcttcaacgaca |
| | ccatcgacccgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctggtacgagc |
| | cctcccagaagtggatcatgaccgcggccaagtcccaggactacaagatcgagatctactcctccgacgacctgaagtcctggaagctg |
| | gagtccgcgttcgccaacgagggcttcctcggctaccagtacgagtgccccggcctgatcgaggtccccaccgagcaggaccccagca |
| | agtcctactgggtgatgttcatctccatcaaccccggcgccccggccggcggctccttcaaccagtacttcgtcggcagcttcaacggcac |
| | ccacttcgaggccttcgacaaccagtcccgcgtggtggacttcggcaaggactactacgccctgcagaccttcttcaacaccgacccgac |
| | ctacgggagcgccctgggcatcgcgtgggcctccaactgggagtactccgccttcgtgcccaccaacccctggcgctcctccatgtcctc |
| | gtgcgcaagttctccctcaacaccgagtaccaggccaacccggagacggagctgatcaacctgaaggccgagccgatcctgaacatc |
| | agcaacgccggcccctggagccggttcgccaccaacaccacgttgacgaaggccaacagctacaacgtcgacctgtccaacagcacc |
| | ggcaccctggagttcgagctggtgtacgccgtcaacaccacccagacgatctccaagtccgtgttcgcggacctctccctctggttcaagg |
| | gcctggaggaccccgaggagtacctccgcatgggcttcgaggtgtccgcgtcctccttcttcctggaccgcgggaacagcaaggtgaag |
| | ttcgtgaaggagaacccctacttcaccaaccgcaatgagcgtgaacaaccagcccttcaagagcgagaacgacctgtcctactacaagg |
| | tgtacggcttgctggaccagaacatcctggagctgtacttcaacgacggcgacgtcgtgtccaccaacacctacttcatgaccaccggga |
| | acgccctgggctccgtgaacatgacgacgggggtggacaacctgttctacatcgacaagttccaggtgcgcgaggtcaagtgactTA |
| | AGACGCCCGCGCGGCGCACCTGACCTGTTCTCTCGAGGGCGCCTGTTCTGCCTTGCGA |
| | AACAAGCCCCTGGAGCATGCGTGCATGATCGTCTCTGGCGCCCCGCCGCGCGGTTTGT |
| | CGCCCTCGCGGGCGCCGCGGCCGCGGGGGCGCATTGAAATTGTTGCAAACCCCACCT |
| | GACAGATTGAGGGCCCAGGCAGGAAGGCGTTGAGATGGAGGTACAGGAGTCAAGTA |
| | ACTGAAAGTTTTTATGATAACTAACAACAAAGGGTCGTTTCTGGCCAGCGAATGACA |
| | AGAACAAGATTCCACATTTCCGTGTAGAGGCTTGCCATCGAATGTGAGCGGGCGGGC |
| | CGCGGACCCGACAAAACCCTTACGACGTGGTAAGAAAAACGTGGCGGGCACTGTCCC |
| | TGTAGCCTGAAGACCAGCAGGAGACGATCGGAAGCATCACAGCACAACCGGtgcagcag |
| | cagctcggatagtatcgacacactctggacgctggtcgtgtgatggactgttgccgccacacttgctgccttgacctgtgaatatccctgcc |
| | gcttttatcaaacagcctcagtgtgtttgatcttgtgtgtacgcgcttttgcgagttgctagctgcttgtgctatttgcgaataccaccccc |
| | agcatccccttccctcgtttcatatcgcttgcatcccaaccgcaacttatctacgctgtcctgctatccctcagcgctgctcctgctcctgc |
| | tcactgcccctcgcacagccttggtttgggctccgcctgtattctcctggtactgcaacctgtaaaccagcactgcaatgctgatgcacggg |
| | 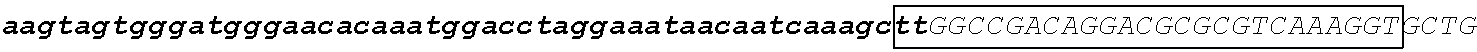 |
| | 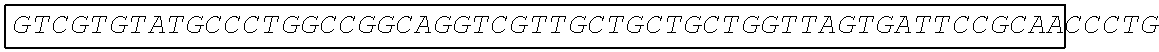 |
| | 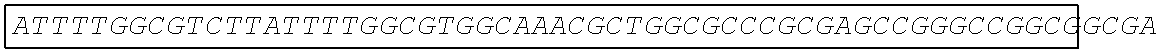 |
| | 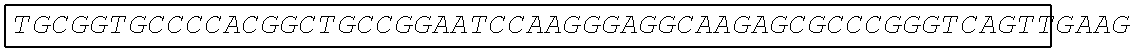 |
| | 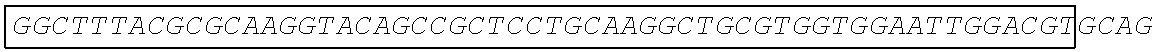 |
| | 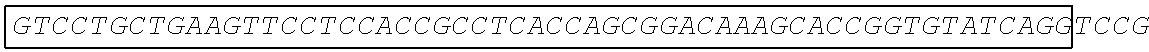 |
| | 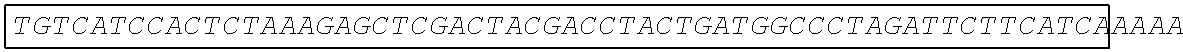 |
| | 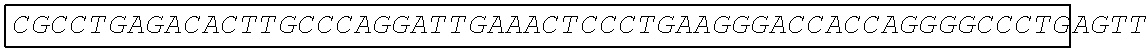 |
| | 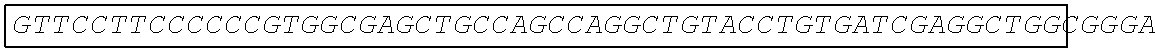 |
| | 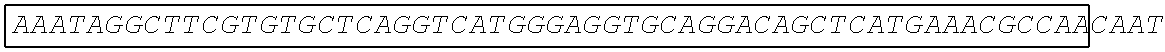 |
| | 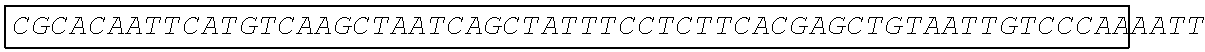 |
| | 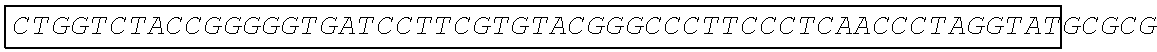 |
| | 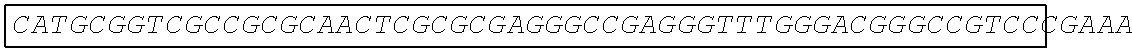 |
| | 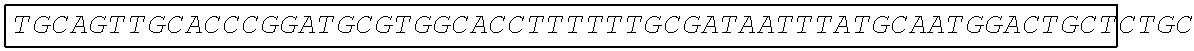 |
| | 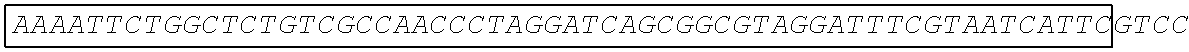 |
| | 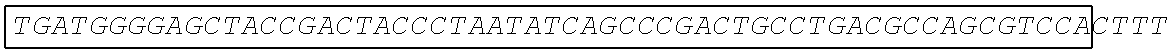 |
| | 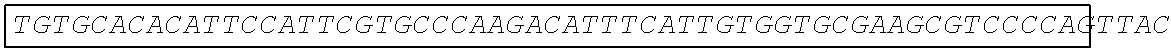 |
| | 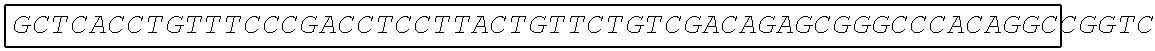 |
| | 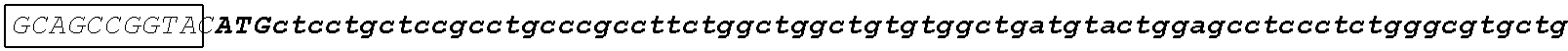 |
| | acgctgtacatccccgtgtggcagctctggggcgcccgcaacgacacgttttactgggtcaagttcctggtgaccgccctgggcatcaagt |
| | tccgcaagcgaggctcgggcgagctgtactctggccaagccccgtgcatctacctggcgaaccaccgctcgtgggcggacttctttgtgg |
| | acgcgtacctcgctggcgggcgcgcgcagatgctgagccggatggcggtgctgtacgcgttcccgatgttcatggcccccgtgatcgcca |
| | tgcggggctgcattgtctttaagcgcggacgcattgatgacaaggaggcgttcaacaagtggatcgacggcctcgtggaggcgtcgccg |
| | cagaacggtctgatggtctaccctgagggccaccgctctacgctggcccactcgctgcccctgaagcgcggcatgctgcactacgcgtac |
| | tcccgcaagatgcccgtgcagattatcatgagcgcgaacaaggagagcgtcctctcggagaaggagctgcacgtgggcttcggttgca |
| | cggtcgtgacgtcttactcgggcctcatctcccccggcgactacgctggcttcgagggcttcatggctgcggtgcaggccgcttgggacaa |
| | ggagtggcgcgacacgatgtcggcggacgcggccgggctgccgcccctgcacgtcagcgggaccattgatgacaaccactacccacc |
| | ccgcctggtggtctcgcaactgtttttcagcctcctggggatcaccctcctgttcgccgcttcttcgattacctggggctggattctgcgagc |
| | caagggcgtcctgggcccccagggtgcgaagatggtggtctcctttctggtcgcgtggttcgcggccagcctggcctactgctacaacggcc |
| | aactgccgaagccccgcctcctgcccgatggctccgaggccaacggcgggggtgctggctggaaggctgggggccgtccgggcggtg |
| | gggccgctgccaagggaggcgcgctgaaggccgctgccgctggcgggggcggaggcggatctcccaagaagtccagcTAGcacg |
| | cccgcgcggcgcacctgacctgttctctcgagggcgcctgttctgccttgcgaaacaagcccctggagcatgcgtgcatgatcgtctctggcg |
| | ccccgccgcgcggtttgtcgccctcgcgggcgccgcggccgcgggggcgcattgaaattgttgcaaaccccacctgacagattgagggcc |
| | caggcaggaaggcgttgagatggaggtacaggagtcaagtaactgaaagtttttatgataactaacaacaaagggtcgtttctggccagcgaa |
| | tgacaagaacaagattccacatttccgtgtagaggcttgccatcgaatgtgagcgggcgggccgcggacccgacaaaacccttacgacgtg |
| | gtaagaaaaacgtggcgggcactgtccctgtagcctgaagaccagcaggagacgatcggaagcatcacagcacaaccggtatcgatagc |
| | gtctgcgtgttgggagctggagtcgtgggcttgacgacggcgctgcagctcttgcaggatgtgcctggcgtgcgcgttcacgtcgtgg |
| | ctgagaaatatggcgacgaaacgttgacggctggggccggcgggctgtggatgccatacgcattgggtacgcggccattggatggg |
| | attggtaggcttatggagggataatagagtttttgtcggatccaacgcatgttgatgcagtagcccggtgtgctgaaagtatggaagg |
| | atggtgcattggctattcacatgcgctgcccaccccttttcgcaggaaatgtgccggcatcgttggtgcaccgatggggaaaatcgac |
| | gttcgaccactacatgaaaatgtatacctctgaagatgcagcaactgcgggtgcgaaacggataacggtttggtaggtgtatgtcgca |
| | gcatgtgctggattttgcgggcaactccccctgccacggcccattgcaggtgtcatgttgactggaggatacgaactttcggccgtcaa |
| | attcccagaggaggaccccctctgggccgacattgtgcccactgctcttccgcttgctgtttcctgtgtgaaattgt |

The construct can be written as 5′DAO1b::CrTUB2:ScSUC2:PmPGH:CvNR:PmAMT03: RsLPAAT2:PmPGH::3′DAO1b. Proceeding in the 5′ to 3′ direction bold, lowercase sequences represent genomic DNA from CHK22 that permit targeted integration at the DAO1b locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text that enables amplification of the RsLPAAT2 gene, followed by a linker, indicated by lowercase, bold italics, in which contains a C. vulgaris nitrate reductase 3′-UTR. The P. moriformis PmAMT03 promoter, indicated by boxed italicized text, drives the expression of the RsLPAAT2. The initiator ATG and terminator TAG codons of the RsLPAAT2 are indicated by uppercase, bold italics, while the remainder of the gene is indicated by bold italics. The P. moriformis PmPGH 3′-UTR is indicated by lowercase underlined text followed by the CHK22 DAO1b genomic region indicated by bold, lowercase text.

Table 44 shows the fatty acid/TAG profiles of primary transformants of CHK22 transformed with pCHK977. The P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 44
Screen of primary transformants of CHK22 with pCHK977.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK977- | 0.06 | 0.04 | 21.34 | 3.17 | 56.63 | 14.69 | 0.57 | 0.24 | 0.1 | 1.46 |
| 1 | | | | | | | | | | |
| pCHK977- | 0.06 | 0.02 | 20.32 | 3.34 | 55.75 | 16.31 | 0.58 | 0.25 | 0.11 | 1.53 |
| 2 | | | | | | | | | | |
| pCHK977- | 0.06 | 0.04 | 19.94 | 3.22 | 56 | 16.65 | 0.6 | 0.24 | 0.12 | 1.48 |
| 3 | | | | | | | | | | |
| pCHK977- | 0.06 | 0.03 | 21.97 | 3.25 | 56.97 | 13.74 | 0.54 | 0.24 | 0.1 | 1.46 |
| 4 | | | | | | | | | | |
| pCHK977- | 0.05 | 0.02 | 25.83 | 2.99 | 59.44 | 7.9 | 0.46 | 0.25 | 0.08 | 1.58 |
| 5 | | | | | | | | | | |
| pCHK977- | 0.04 | 0.02 | 26.09 | 3.12 | 59.68 | 7.43 | 0.44 | 0.27 | 0.08 | 1.55 |
| 6 | | | | | | | | | | |
| CHK22 | 0.04 | 0.02 | 26.18 | 2.95 | 59.62 | 7.55 | 0.46 | 0.24 | 0.07 | 1.49 |
| Control | | | | | | | | | | |

### Example 22: Expressing Chlorella desiccata LPAAT2 (ChdeLPAAT2) in P. moriformis Strain CHK22

ChdeLPAAT2 was introduced into a P. moriformis strain CHK22. The expression construct pCHK978 contained 5′ and 3′ homology arms to permit targeted integration of ChdeLPAAT2 into the genome and is shown in Table 45.

TABLE 45
Integrative sequences for the transformation of P. moriformis with pCHK978 encoding ChdeLPAAT2.

| SEQ ID | |
|---|---|
| NO: | Nucleotide Sequence |
| 34 | gctcttccgcttgcccgcaccctcgttgatctgggagccctgcgcagccccttaaatcatctcagtcaggtttctatgttcaactgagcct |
| | aaagggctttcgtcacgcgcacgagcacacgtatatcggctacgcagtctctcagaagcggtagaacagttcgcaagccctcgtcgg |
| | tcgaaaacttgcgccagtactattgaattaaattaaatgatcgaatgagacgcgaaacttttgcagaatgccactgagtttgcccaga |
| | gaatgggagtggcgccattcaccatccgcctgtgcacggcctgattcgccgagacgatgaatggcgagaccagggagcggcttgcg |
| | agccccgagccggtagcaggaataacggtcgacaatcttcctgtccaattactggcaaccattagaaagagccggagcgcgttgaa |
| | attctgcaatcgagtaatttttcgatgcggcgggcctgctgaaccctaaggctccggcctatgtttaaggcgatccaagatgcacgcgg |
| | ccccaggcacgtgtctcaagcacaaaccccagccttagtttcgagactttgggagatagcggcagatatctagtttggcattttgtatat |
| | taattaagtctcgcaatggagcgctctgatgcggtgcagcgtcggctgcagcacctggcagtggcgctggggtcgccctatcgctcgg |
| | 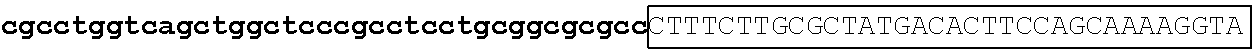 |
| | 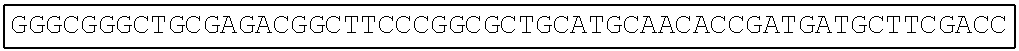 |
| | 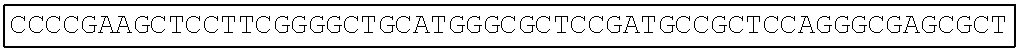 |
| | 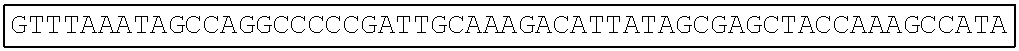 |
| | 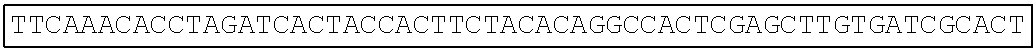 |
| | 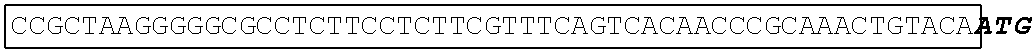 |
| | ctgctgcaggccttcctgttcctgctggccggcttcgccgccaagatcagcgcctccatgacgaacgagacgtccgaccgccccctggtg |
| | cacttcacccccaacaagggctggatgaacgaccccaacggcctgtggtacgacgagaaggacgccaagtggcacctgtacttccagt |
| | acaacccgaacgacaccgtctgggggacgcccttgttctgggggccacgccacgtccgacgacctgaccaactgggaggaccagccca |
| | tcgccatcgccccgaagcgcaacgactccggcgccttctccggctccatggtggtggactacaacaacacctccggcttcttcaacgaca |
| | ccatcgacccgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctggtacgagc |
| | cctcccagaagtggatcatgaccgcggccaagtcccaggactacaagatcgagatctactcctccgacgacctgaagtcctggaagctg |
| | gagtccgcgttcgccaacgagggcttcctcggctaccagtacgagtgccccggcctgatcgaggtccccaccgagcaggaccccagca |
| | agtcctactgggtgatgttcatctccatcaaccccggcgccccggccggcggctccttcaaccagtacttcgtcggcagcttcaacggcac |
| | ccacttcgaggccttcgacaaccagtcccgcgtggtggacttcggcaaggactactacgccctgcagaccttcttcaacaccgacccgac |
| | ctacgggagcgccctgggcatcgcgtgggcctccaactgggagtactccgccttcgtgcccaccaacccctggcgctcctccatgtccctc |
| | gtgcgcaagttctccctcaacaccgagtaccaggccaacccggagacggagctgatcaacctgaaggccgagccgatcctgaacatc |
| | agcaacgccggcccctggagccggttcgccaccaacaccacgttgacgaaggccaacagctacaacgtcgacctgtccaacagcacc |
| | ggcaccctggagttcgagctggtgtacgccgtcaacaccacccagacgatctccaagtccgtgttcgcggacctctccctctggttcaagg |
| | gcctggaggaccccgaggagtacctccgcatgggcttcgaggtgtccgcgtcctccttcttcctggaccgcgggaacagcaaggtgaag |
| | ttcgtgaaggagaacccctacttcaccaaccgcatgagcgtgaacaaccagcccttcaagagcgagaacgacctgtcctactacaagg |
| | tgtacggcttgctggaccagaacatcctggagctgtacttcaacgacggcgacgtcgtgtccaccaacacctacttcatgaccaccggga |
| | acgccctgggctccgtgaacatgacgacgggggtggacaacctgttctacatcgacaagttccaggtgcgcgaggtcaagtgactTA |
| | AGACGCCCGCGCGGCGCACCTGACCTGTTCTCTCGAGGGCGCCTGTTCTGCCTTGCGA |
| | AACAAGCCCCTGGAGCATGCGTGCATGATCGTCTCTGGCGCCCCGCCGCGCGGTTTGT |
| | CGCCCTCGCGGGCGCCGCGGCCGCGGGGGCGCATTGAAATTGTTGCAAACCCCACCT |
| | GACAGATTGAGGGCCCAGGCAGGAAGGCGTTGAGATGGAGGTACAGGAGTCAAGTA |
| | ACTGAAAGTTTTTATGATAACTAACAACAAAGGGTCGTTTCTGGCCAGCGAATGACA |
| | AGAACAAGATTCCACATTTCCGTGTAGAGGCTTGCCATCGAATGTGAGCGGGCGGGC |
| | CGCGGACCCGACAAAACCCTTACGACGTGGTAAGAAAAACGTGGCGGGCACTGTCCC |
| | TGTAGCCTGAAGACCAGCAGGAGACGATCGGAAGCATCACAGCACAACCGGtgcagcag |
| | cagctcggatagtatcgacacactctggacgctggtcgtgtgatggactgttgccgccacacttgctgccttgacctgtgaatatccctgcc |
| | gcttttatcaaacagcctcagtgtgtttgatcttgtgtgtacgcgcttttgcgagttgctagctgcttgtgctatttgcgaataccaccccc |
| | agcatccccttccctcgtttcatatcgcttgcatcccaaccgcaacttatctacgctgtcctgctatccctcagcgctgctcctgctcctgc |
| | tcactgcccctcgcacagccttggtttgggctccgcctgtattctcctggtactgcaacctgtaaaccagcactgcaatgctgatgcacggg |
| | 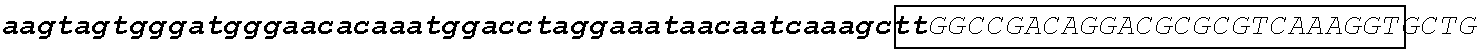 |
| | 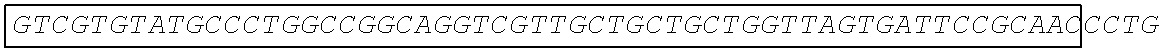 |
| | 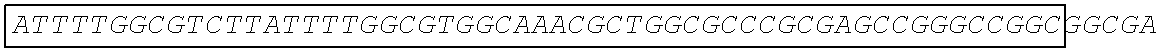 |
| | 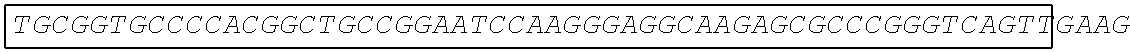 |
| | 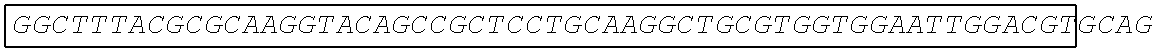 |
| | 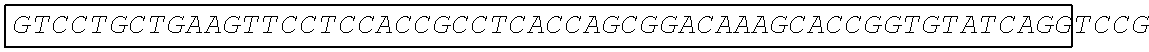 |
| | 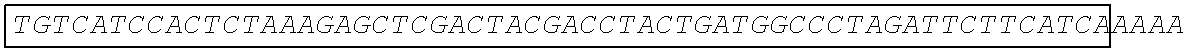 |
| | 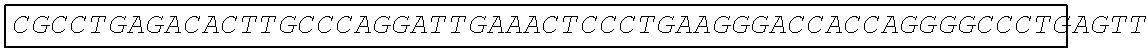 |
| | 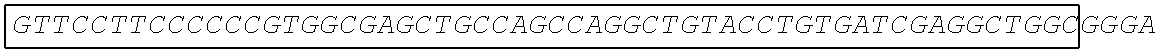 |
| | 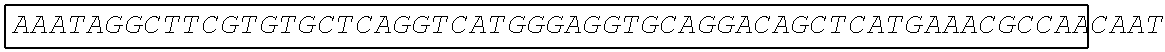 |
| | 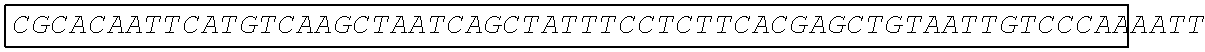 |
| | 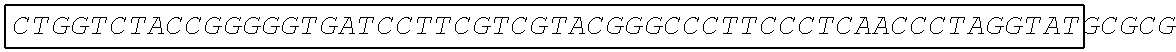 |
| | 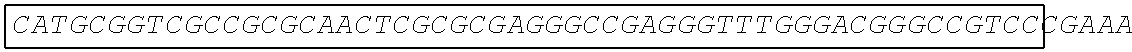 |
| | 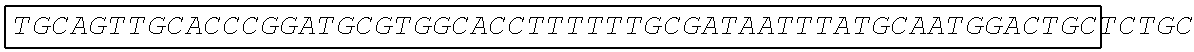 |
| | 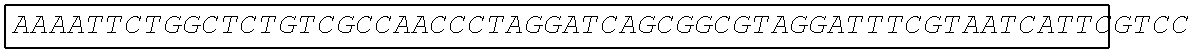 |
| | 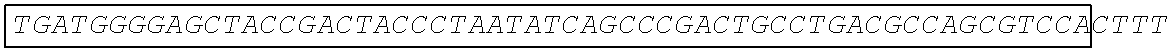 |
| | 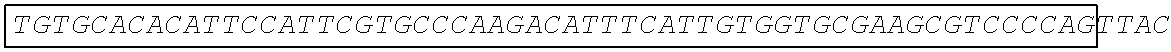 |
| | 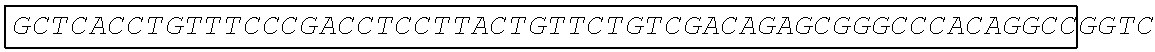 |
| | 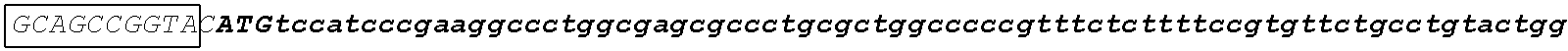 |
| | gacgtggctatcctggtcctgacctaccggatcaagtggctggactttaagaagaagcgaaacgacgtgctcgccctgggcaagggcct |
| | gaaggacatccaccgagtgaccatcctgaaggtgggctaccgtgagctgtaccgcaagaactgcatgtacctgtgcaaccaccgtagct |
| | gggcggacttcctcgtcgatcagtacgtcacggagggccggaccctcttcgtcggccgttgggccgtcgccgcggcttttcccctggtcatg |
| | atccccctgcgggccatccggtgcgcgatcctctttaagcgcggacacatcgcggacatcgagggattcaacaagtggattgactccca |
| | gttcgctaacagcccgcaaacgggtctgggtgtgtaccccgagggccatcgttcgacccacggcgagtcgctccctctgaagcgaggga |
| | tgctgaagtacgccttcagccgaaagctcccggtgcagatcgtgatcgggggcaacaaggagtcgatcctgtccgagaagcactgcac |
| | ggcgcgtttctaccagaccatcgcggtcggtttctccgaggtcctgaagcctgaggagtacgatgactttgagaacttcatgcagaaggtc |
| | caggagacgtggaaccgtgagtggagcgaggtcttctcgtccaacctgaagggcctcccggtgctgccggaggtccgggagccccaa |
| | ctggactacccaatggatgtgcgcatgattatggcgatctgcgcggtggtcaacatcctcatgctggcgggcgtggcctggctgacctggt |
| | gggccgtggcggccgtgatggcgctcatgggccccctgcaatggccagtcgctggcctggtcgtggcctacatcgtcgcgtctttttacgtg |
| | tactcgaaggctgatgacgtgcgtcgcacccaccaaaagatgttccaacagcggaagcaggcgcccccgattgtctccgctgcgggcg |
| | ctggcgagggcgccggggagggcgccggagaggagaacggcgacaagaaggagcagTAGcacgcccgcgcggcgcacctgac |
| | ctgttctctcgagggcgcctgttctgccttgcgaaacaagcccctggagcatgcgtgcatgatcgtctctggcgccccgccgcgcggtttgtcg |
| | ccctcgcgggcgccgcggccgcgggggcgcattgaaattgttgcaaaccccacctgacagattgagggcccaggcaggaaggcgttgag |
| | atggaggtacaggagtcaagtaactgaaagtttttatgataactaacaacaaagggtcgtttctggccagcgaatgacaagaacaagattccac |
| | atttccgtgtagaggcttgccatcgaatgtgagcgggcgggccgcggacccgacaaaacccttacgacgtggtaagaaaaacgtggcggg |
| | cactgtccctgtagcctgaagaccagcaggagacgatcggaagcatcacagcacaaccggtatcgatagcgtctgcgtgttgggagctgg |
| | agtcgtgggcttgacgacggcgctgcagctcttgcaggatgtgcctggcgtgcgcgttcacgtcgtggctgagaaatatggcgacga |
| | aacgttgacggctggggccggcgggctgtggatgccatacgcattgggtacgcggccattggatgggattggtaggcttatggaggg |
| | ataatagagtttttgtcggatccaacgcatgttgatgcagtagcccggtgtgctgaaagtatggaaggatggtgcattggctattcaca |
| | tgcgctgcccaccccttttcgcaggaaatgtgccggcatcgttggtgcaccgatggggaaaatcgacgttcgaccactacatgaaaat |
| | gtatacctctgaagatgcagcaactgcgggtgcgaaacggataacggtttggtaggtgtatgtcgcagcatgtgctggattttgcggg |
| | caactccccctgccacggcccattgcaggtgtcatgttgactggaggatacgaactttcggccgtcaaattcccagaggaggaccccc |
| | tctgggccgacattgtgcccactgctcttccgcttgctgtttcctgtgtgaaattgt |

The construct can be written as 5′DAO1b::CrTUB2:ScSUC2:PmPGH:CvNR:PmAMT03:ChdeLPAAT2:PmPGH::3′DAO1b. Proceeding in the 5′ to 3′ direction bold, lowercase sequences represent genomic DNA from CHK22 that permit targeted integration at the DAO1b locus via homologous recombination, the C. reinharditi β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text that enables amplification of the ChdeLPAAT2 gene, followed by a linker, indicated by lowercase, bold italics, in which contains a C. vulgaris nitrate reductase 3′-UTR. The P. moriformis PmAMT03 promoter, indicated by boxed italicized text, drives the expression of the ChdeLPAAT2. The initiator ATG and terminator TAG codons of the ChdeLPAAT2 are indicated by uppercase, bold italics, while the remainder of the gene is indicated by bold italics. The P. moriformis PmPGH 3′-UTR is indicated by lowercase underlined text followed by the CHK22 DAO1b genomic region indicated by bold, lowercase text.

Table 46 shows the fatty acid/TAG profiles of primary transformants of CHK22 transformed with pCHK978. The P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 46
Screen of primary transformants of CHK22 with pCHK978.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK978- | 0.04 | 0.02 | 25.77 | 3.1 | 60.01 | 7.39 | 0.48 | 0.26 | 0.08 | 1.72 |
| 1 | | | | | | | | | | |
| pCHK978- | 0.05 | 0.04 | 24.4 | 3.07 | 56.47 | 11.66 | 0.52 | 0.34 | 0.1 | 1.70 |
| 2 | | | | | | | | | | |
| pCHK978- | 0.06 | 0.03 | 21.29 | 3.2 | 59.24 | 12.16 | 0.55 | 0.29 | 0.1 | 1.55 |
| 3 | | | | | | | | | | |
| pCHK978- | 0.06 | 0.03 | 21.03 | 3.39 | 59.09 | 12.34 | 0.55 | 0.31 | 0.11 | 1.56 |
| 4 | | | | | | | | | | |
| pCHK978- | 0.05 | 0.02 | 22.31 | 3.05 | 59.53 | 11.05 | 0.53 | 0.28 | 0.1 | 1.56 |
| 5 | | | | | | | | | | |
| pCHK978- | 0.05 | 0.03 | 21.5 | 3.18 | 59.22 | 11.9 | 0.56 | 0.29 | 0.11 | 1.67 |
| 6 | | | | | | | | | | |
| CHK22 | 0.04 | 0.02 | 26.18 | 2.95 | 59.62 | 7.55 | 0.46 | 0.24 | 0.07 | 1.49 |
| Control | | | | | | | | | | |

### Example 23: Expressing Auxenochlorella protothecoides LPAAT2 (ApLPAAT2) in P. moriformis Strain CHK22

ApLPAAT2 was introduced into a P. moriformis strain CHK22. The expression construct pCHK979 contained 5Y and 3Y homology arms to permit targeted integration of ApLPAAT2 into the genome and is shown in Table 47.

TABLE 47
Integrative sequences for the transformation of P. moriformis with pCHK979
encoding ApLPAAT2.

| SEQ | |
|---|---|
| ID | |
| NO: | Nucleotide Sequence |
| 35 | gctcttccgcttgcccgcaccctegttgatctgggagccctgcgcagccccttaaatcatctcagtcaggtttctatgttcaactgagcct |
| | aaagggctttcgtcacgcgcacgagcacacgtatatcggctacgcagtctctcagaagcggtagaacagttcgcaagccctcgtcgg |
| | tcgaaaacttgcgccagtactattgaattaaattaaatgatcgaatgagacgcgaaacttttgcagaatgccactgagtttgcccaga |
| | gaatgggagtggcgccattcaccatccgcctgtgcacggcctgattcgccgagacgatgaatggcgagaccagggagcggcttgcg |
| | agccccgagccggtagcaggaataacggtcgacaatcttcctgtccaattactggcaaccattagaaagagccggagcgcgttgaa |
| | attctgcaatcgagtaatttttcgatgcggcgggcctgctgaaccctaaggctccggcctatgtttaaggcgatccaagatgcacgcgg |
| | ccccaggcacgtgtctcaagcacaaaccccagccttagtttcgagactttgggagatagcggcagatatctagtttggcattttgtatat |
| | taattaagtctcgcaatggagcgctctgatgcggtgcagcgtcggctgcagcacctggcagtggcgctggggtcgccctatcgctcgg |
| | 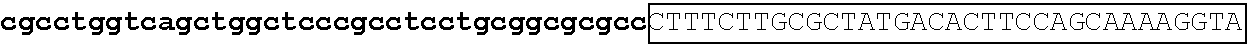 |
| | 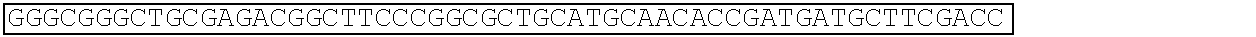 |
| | 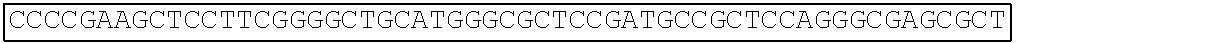 |
| | 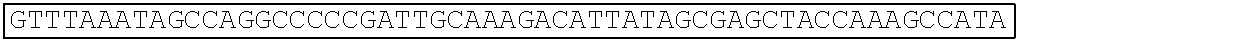 |
| | 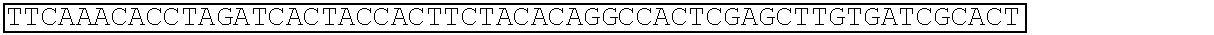 |
| | 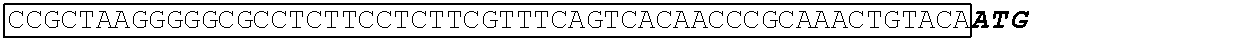 |
| | ctgctgcaggccttcctgttcctgctggccggcttcgccgccaagatcagcgcctccatgacgaacgagacgtccgaccgccccctggtg |
| | cacttcacccccaacaagggctggatgaacgaccccaacggcctgtggtacgacgagaaggacgccaagtggcacctgtacttccagt |
| | acaacccgaacgacaccgtctgggggacgcccttgttctggggccacgccacgtccgacgacctgaccaactgggaggaccagccca |
| | tcgccatcgccccgaagcgcaacgactccggcgccttctccggctccatggtggtggactacaacaacacctccggcttcttcaacgaca |
| | ccatcgacccgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctggtacgagc |
| | cctcccagaagtggatcatgaccgcggccaagtcccaggactacaagatcgagatctactcctccgacgacctgaagtcctggaagctg |
| | gagtccgcgttcgccaacgagggcttcctcggctaccagtacgagtgccccggcctgatcgaggtccccaccgagcaggaccccagca |
| | agtcctactgggtgatgttcatctccatcaaccccggcgccccggccggcggctccttcaaccagtacttcgtcggcagcttcaacggcac |
| | ccacttcgaggccttcgacaaccagtcccgcgtggtggacttcggcaaggactactacgccctgcagaccttcttcaacaccgacccgac |
| | ctacgggagcgccctgggcatcgcgtgggcctccaactgggagtactccgccttcgtgcccaccaacccctggcgctcctccatgtccctc |
| | gtgcgcaagttctccctcaacaccgagtaccaggccaacccggagacggagctgatcaacctgaaggccgagccgatcctgaacatc |
| | agcaacgccggcccctggagccggttcgccaccaacaccacgttgacgaaggccaacagctacaacgtcgacctgtccaacagcacc |
| | ggcaccctggagttcgagctggtgtacgccgtcaacaccacccagacgatctccaagtccgtgttcgcggacctctccctctggttcaagg |
| | gcctggaggaccccgaggagtacctccgcatgggcttcgaggtgtccgcgtcctccttcttcctggaccgcgggaacagcaaggtgaag |
| | ttcgtgaaggagaacccctacttcaccaaccgcatgagcgtgaacaaccagcccttcaagagcgagaacgacctgtcctactacaagg |
| | tgtacggcttgctggaccagaacatcctggagctgtacttcaacgacggcgacgtcgtgtccaccaacacctacttcatgaccaccggga |
| | acgccctgggctccgtgaacatgacgacgggggtggacaacctgttctacatcgacaagttccaggtgcgcgaggtcaagtgactTA |
| | AGACGCCCGCGCGGCGCACCTGACCTGTTCTCTCGAGGGCGCCTGTTCTGCCTTGCGA |
| | AACAAGCCCCTGGAGCATGCGTGCATGATCGTCTCTGGCGCCCCGCCGCGCGGTTTGT |
| | CGCCCTCGCGGGCGCCGCGGCCGCGGGGGCGCATTGAAATTGTTGCAAACCCCACCT |
| | GACAGATTGAGGGCCCAGGCAGGAAGGCGTTGAGATGGAGGTACAGGAGTCAAGTA |
| | ACTGAAAGTTTTTATGATAACTAACAACAAAGGGTCGTTTCTGGCCAGCGAATGACA |
| | AGAACAAGATTCCACATTTCCGTGTAGAGGCTTGCCATCGAATGTGAGCGGGCGGGC |
| | CGCGGACCCGACAAAACCCTTACGACGTGGTAAGAAAAACGTGGCGGGCACTGTCCC |
| | TGTAGCCTGAAGACCAGCAGGAGACGATCGGAAGCATCACAGCACAACCGGtgcagcag |
| | cagctcggatagtatcgacacactctggacgctggtcgtgtgatggactgttgccgccacacttgctgccttgacctgtgaatatccctgcc |
| | gcttttatcaaacagcctcagtgtgtttgatcttgtgtgtacgcgcttttgcgagttgctagctgcttgtgctatttgcgaataccacccccagca |
| | tccccttccctcgtttcatategcttgcatcccaaccgcaacttatctacgctgtcctgctatccctcagegctgctcctgctcctgctcactgcc |
| | cctcgcacagccttggtttgggctccgcctgtattctcctggtactgcaacctgtaaaccagcactgcaatgctgatgcacgggaagtagtg |
| | 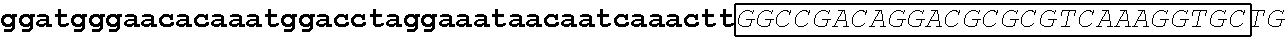 |
| | 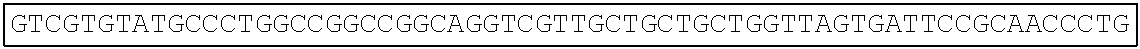 |
| | 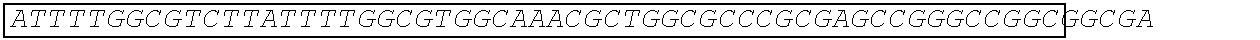 |
| | 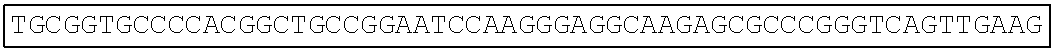 |
| | 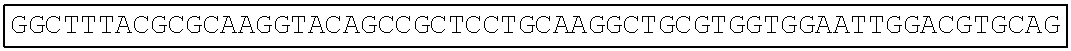 |
| | 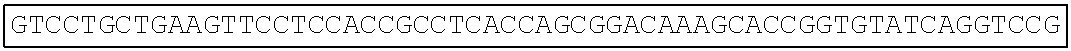 |
| | 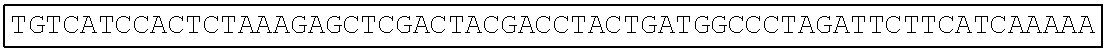 |
| | 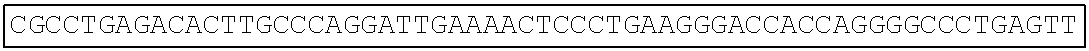 |
| | 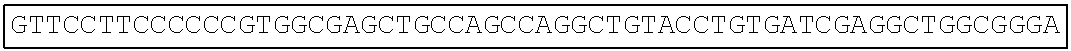 |
| | 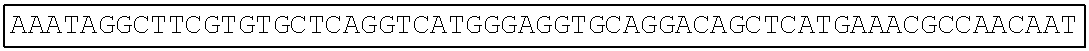 |
| | 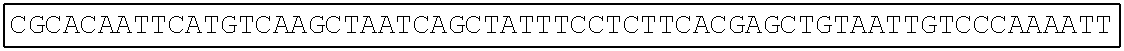 |
| | 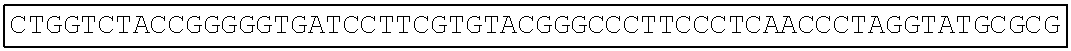 |
| | 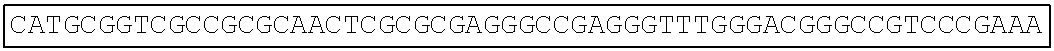 |
| | 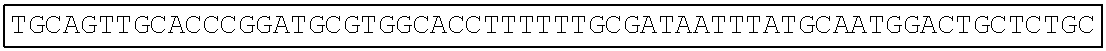 |
| | 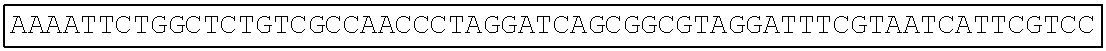 |
| | 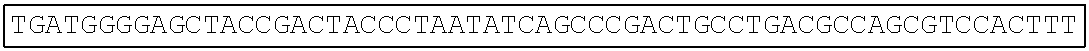 |
| | 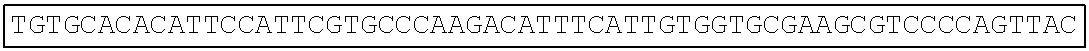 |
| | 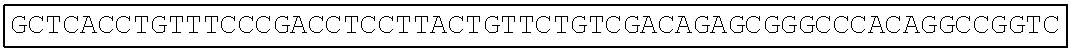 |
| | 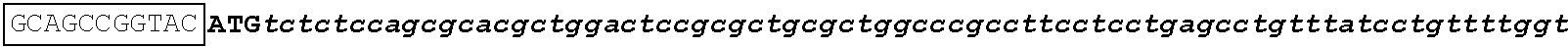 |
| | ccgtccccttctgctcgctgacctactacatccgtttcctggacctcaagaccgagcgccggggagatgtgtttagctgggccgtcggcatg |
| | aagcggtggttcggcatcaagatcctgaaggtgggcgaccgcgaactcttccgcaaggactgcatgtacctgatcaaccatcggtcgtg |
| | gggcgacttcctggtcgaccagtacctcacggagggccgatcgacctttctggcgcgcgcgatggtggcggtggcgttcccgttctttatg |
| | acggtcatcatggtgatgaaggccaccatggtctttaagcgcggcaacatcccagacaaggagaagttcaaccgctggatcgactccg |
| | agtttgcggccacgccccagaccgccatcgctgtgtacccggagggccatcggtcgaccatgaccgactcgctccccctgaagcgcggt |
| | atgctgcattacgcttactcccgcaagctcccggtccaggtgattatcgcggccaacaaggaggccgtgctgagcgagaagcacagca |
| | ccgcccgcttcgggcagacgatctggggggctactccgagcccatgtttcccgagaagtacgagaccttcgacaagttcatggacaag |
| | gtccaggccacgtggaacgaggagtggaacaccgtctttggcaccaagccggagggtctgcgcgagctggtctgcgaggacccgaag |
| | ctcctgacgcccctgcacgtgcgtcgaggtgtctccacgatcattgcggtggtcgacatctgctttctggcgatcatgtactacagcctgcgg |
| | atggcccgccgggtgtccgcggccctggggccgctggaggcgcccctcctggccctgctcgcggtctacgtgaccgcgagcctcgtgcg |
| | ctactctegcccctacgacggcgtggccgcgtctgctcgcaagggcaagctggcgcaggegagegccacccaagegcccccggegcc |
| | caagctggccgcgagcggagtggccgacgtgtccgeggccaccatcaaccgtgctgcctccctegctgccgagccaggcgatccgca |
| | gggacgccagTGAcacgcccgcgcggcgcacctgacctgttctctcgagggcgcctgttctgccttgcgaaacaagcccctggagcatg |
| | cgtgcatgatcgtctctggcgccccgccgcgcggtttgtcgccctcgcgggcgccgcggccgcgggggcgcattgaaattgttgcaaaccc |
| | cacctgacagattgagggcccaggcaggaaggcgttgagatggaggtacaggagtcaagtaactgaaagtttttatgataactaacaacaaa |
| | gggtcgtttctggccagcgaatgacaagaacaagattccacatttccgtgtagaggcttgccatcgaatgtgagcgcggaccc |
| | gacaaaacccttacgacgtggtaagaaaaacgtgggggcactgtccctgtagcctgaagaccagcaggagacgatoggaagcatcacag |
| | cacaaccggtatcgatagcgtctgcgtgttgggagctggagtcgtgggcttgacgacggcgctgcagctcttgcaggatgtgcctggc |
| | gtgcgcgttcacgtcgtggctgagaaatatggcgacgaaacgttgacggctggggccggcgggctgtggatgccatacgcattgggt |
| | acgcggccattggatgggattggtaggcttatggagggataatagagtttttgtcggatccaacgcatgttgatgcagtagcccggtgt |
| | gctgaaagtatggaaggatggtgcattggctattcacatgcgctgcccaccccttttcgcaggaaatgtgccggcatcgttggtgcacc |
| | gatggggaaaatcgacgttcgaccactacatgaaaatgtatacctctgaagatgcagcaactgcgggtgcgaaacggataacggttt |
| | ggtaggtgtatgtcgcagcatgtgctggattttgcgggcaactccccctgccacggcccattgcaggtgtcatgttgactggaggatac |
| | gaacttteggccgtcaaattcccagaggaggaccccctctgggccgacattgtgcccactgctcttccgcttgctgtttcctgtgtgaaa |
| | ttgt |

The construct can be written as 5′DAO1b::CrTUB2:ScSUC2:PmPGH:CvNR:PmAMT03:ApLPAAT2:PmPGH::3′DAO1b. Proceeding in the 5′ to 3′ direction bold, lowercase sequences represent genomic DNA from CHK22 that permit targeted integration at the DAO1b locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text that enables amplification of the ApLPAAT2 gene, followed by a linker, indicated by lowercase, bold italics, in which contains a C. vulgaris nitrate reductase 3′-UTR. The P. moriformis PmAMT03 promoter, indicated by boxed italicized text, drives the expression of the ApLPAAT2. The initiator ATG and terminator TAG codons of the ApLPAAT2 are indicated by uppercase, bold italics, while the remainder of the gene is indicated by bold italics. The P. moriformis PmPGH 3′-UTR is indicated by lowercase underlined text followed by the CHK22 DAO1b genomic region indicated by bold, lowercase text.

Table 48 shows the fatty acid/TAG profiles of primary transformants of CHK22 transformed with pCHK979. The P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 48
Screen of primary transformants of CHK22 with pCHK979.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK979- | 0.04 | 0.03 | 25.91 | 3.07 | 60.15 | 7.22 | 0.46 | 0.26 | 0.08 | 1.47 |
| 1 | | | | | | | | | | |
| pCHK979- | 0.06 | 0.04 | 21.27 | 3.41 | 57.2 | 14.02 | 0.54 | 0.26 | 0.11 | 1.58 |
| 2 | | | | | | | | | | |
| pCHK979- | 0.05 | 0.05 | 21.35 | 3.6 | 58.07 | 12.97 | 0.52 | 0.28 | 0.11 | 1.58 |
| 3 | | | | | | | | | | |
| pCHK979- | 0.06 | 0.03 | 21.43 | 3.39 | 58.06 | 13.06 | 0.54 | 0.27 | 0.1 | 1.55 |
| 4 | | | | | | | | | | |
| pCHK979- | 0.07 | 0.04 | 20.44 | 3.54 | 56.79 | 15.01 | 0.58 | 0.27 | 0.11 | 1.56 |
| 5 | | | | | | | | | | |
| pCHK979- | 0.05 | 0.03 | 24.9 | 3.03 | 59.02 | 9.16 | 0.48 | 0.25 | 0.08 | 1.54 |
| 6 | | | | | | | | | | |
| CHK22 | 0.04 | 0.02 | 26.18 | 2.95 | 59.62 | 7.55 | 0.46 | 0.24 | 0.07 | 1.49 |
| Control | | | | | | | | | | |

### Example 24: Expressing Chloropicon primus LPAAT2 (ChprLPAAT2) in P. moriformis Strain CHK22

ChprLPAAT2 was introduced into a P. moriformis strain CHK22. The expression construct pCHK980 contained 5Y and 3Y homology arms to permit targeted integration of ChprLPAAT2 into the genome and is shown in Table 49.

TABLE 49
Integrative sequences for the transformation of P. moriformis with pCHK980
encoding ChprLPAAT2.

| SEQ | |
|---|---|
| ID | |
| NO: | Nucleotide Sequence |
| 36 | gctcttccgcttgcccgcaccctcgttgatctgggagccctgcgcagccccttaaatcatctcagtcaggtttctatgttcaactgagcct |
| | aaagggctttcgtcacgcgcacgagcacacgtatatcggctacgcagtctctcagaagcggtagaacagttcgcaagccctcgtcgg |
| | tcgaaaacttgcgccagtactattgaattaaattaaatgatcgaatgagacgcgaaacttttgcagaatgccactgagtttgcccaga |
| | gaatgggagtggcgccattcaccatccgcctgtgcacggcctgattcgccgagacgatgaatggcgagaccagggagcggcttgcg |
| | agccccgagccggtagcaggaataacggtcgacaatcttcctgtccaattactggcaaccattagaaagagccggagcgcgttgaa |
| | attctgcaatcgagtaatttttcgatgcgggggcctgctgaaccctaaggctccggcctatgtttaaggcgatccaagatgcacgcgg |
| | ccccaggcacgtgtctcaagcacaaaccccagccttagtttcgagactttgggagatagcggcagatatctagtttggcattttgtatat |
| | taattaagtctcgcaatggagcgctctgatgcggtgcagcgtcggctgcagcacctggcagtggcgctggggtegccctatcgctcgg |
| | 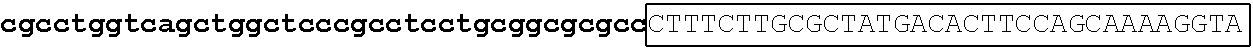 |
| | 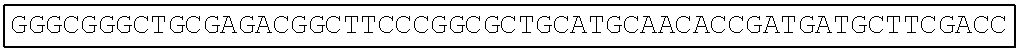 |
| | 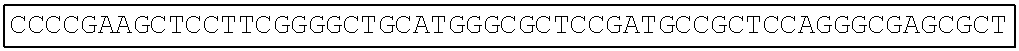 |
| | 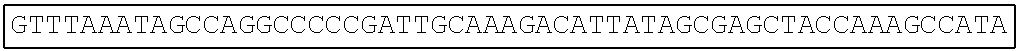 |
| | 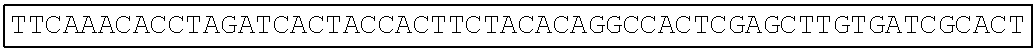 |
| | 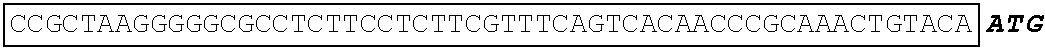 |
| | ctgctgcaggccttcctgttcctgctggccggcttcgccgccaagatcagcgcctccatgacgaacgagacgtccgaccgccccctggtg |
| | cacttcacccccaacaagggctggatgaacgaccccaacggcctgtggtacgacgagaaggacgccaagtggcacctgtacttccagt |
| | acaacccgaacgacaccgtctgggggacgcccttgttctggggccacgccacgtccgacgacctgaccaactgggaggaccagccca |
| | tcgccatcgccccgaagcgcaacgactccggcgccttctccggctccatggtggtggactacaacaacacctccggcttcttcaacgaca |
| | ccatcgacccgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctggtacgagc |
| | cctcccagaagtggatcatgaccgcggccaagtcccaggactacaagatcgagatctactcctccgacgacctgaagtcctggaagctg |
| | gagtccgcgttcgccaacgagggcttcctcggctaccagtacgagtgccccggcctgatcgaggtccccaccgagcaggaccccagca |
| | agtcctactgggtgatgttcatctccatcaaccccggcgccccggccggggctccttcaaccagtacttcgtcggcagcttcaacggcac |
| | ccacttcgaggccttcgacaaccagtcccgcgtggtggacttcggcaaggactactacgccctgcagaccttcttcaacaccgacccgac |
| | ctacgggagcgccctgggcatcgcgtgggcctccaactgggagtactccgccttcgtgcccaccaacccctggcgctcctccatgtccctc |
| | gtgcgcaagttctccctcaacaccgagtaccaggccaacccggagacggagctgatcaacctgaaggccgagccgatcctgaacatc |
| | agcaacgccggcccctggagccggttcgccaccaacaccacgttgacgaaggccaacagctacaacgtcgacctgtccaacagcacc |
| | ggcaccctggagttcgagctggtgtacgccgtcaacaccacccagacgatctccaagtccgtgttcgcggacctctccctctggttcaagg |
| | gcctggaggaccccgaggagtacctccgcatgggcttcgaggtgtccgcgtcctccttcttcctggaccgcgggaacagcaaggtgaag |
| | ttcgtgaaggagaacccctacttcaccaaccgcatgagcgtgaacaaccagcccttcaagagcgagaacgacctgtcctactacaagg |
| | tgtacggcttgctggaccagaacatcctggagctgtacttcaacgacggcgacgtcgtgtccaccaacacctacttcatgaccaccggga |
| | acgccctgggctccgtgaacatgacgacgggggtggacaacctgttctacatcgacaagttccaggtgcgcgaggtcaagtgactTA |
| | AGACGCCCGCGCGGCGCACCTGACCTGTTCTCTCGAGGGCGCCTGTTCTGCCTTGCGA |
| | AACAAGCCCCTGGAGCATGCGTGCATGATCGTCTCTGGCGCCCCGCCGCGCGGTTTGT |
| | CGCCCTCGCGGGCGCCGCGGCCGCGGGGGCGCATTGAAATTGTTGCAAACCCCACCT |
| | GACAGATTGAGGGCCCAGGCAGGAAGGCGTTGAGATGGAGGTACAGGAGTCAAGTA |
| | ACTGAAAGTTTTTATGATAACTAACAACAAAGGGTCGTTTCTGGCCAGCGAATGACA |
| | AGAACAAGATTCCACATTTCCGTGTAGAGGCTTGCCATCGAATGTGAGCGGGCGGGC |
| | CGCGGACCCGACAAAACCCTTACGACGTGGTAAGAAAAACGTGGCGGGCACTGTCCC |
| | TGTAGCCTGAAGACCAGCAGGAGACGATCGGAAGCATCACAGCACAACCGGtgcagcag |
| | cagctcggatagtatcgacacactctggacgctggtcgtgtgatggactgttgccgccacacttgctgccttgacctgtgaatatccctgcc |
| | gcttttatcaaacagcctcagtgtgtttgatcttgtgtgtacgcgcttttgcgagttgctagctgcttgtgctatttgcgaataccacccccagca |
| | tccccttccctcgtttcatategcttgcatcccaaccgcaacttatctacgctgtcctgctatccctcagegctgctcctgctcctgctcactgcc |
| | cctcgcacagccttggtttgggctccgcctgtattctcctggtactgcaacctgtaaaccagcactgcaatgctgatgcacgggaagtagtg |
| | 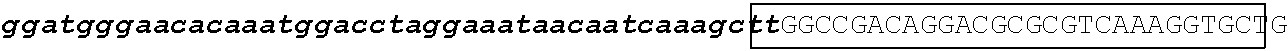 |
| | 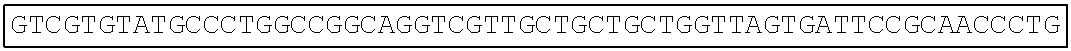 |
| | 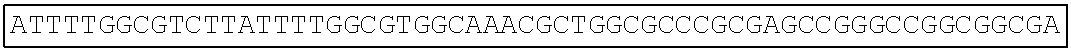 |
| | 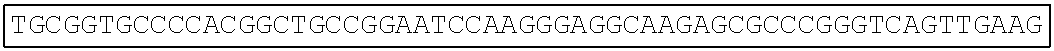 |
| | 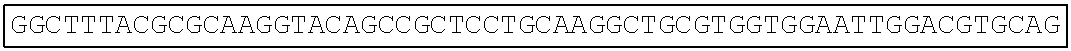 |
| | 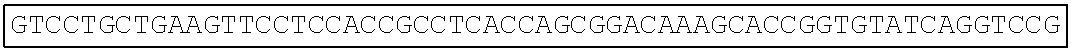 |
| | 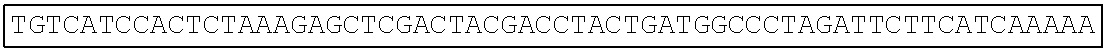 |
| | 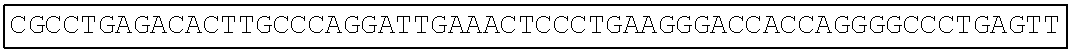 |
| | 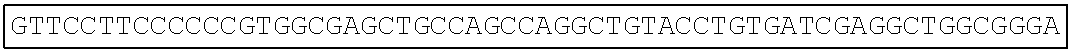 |
| | 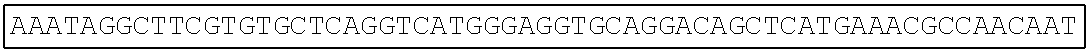 |
| | 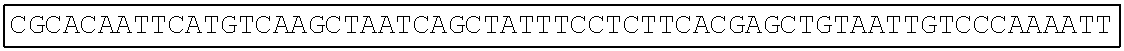 |
| | 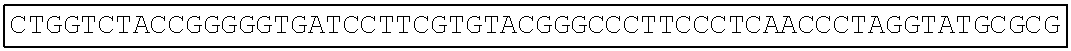 |
| | 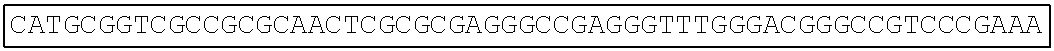 |
| | 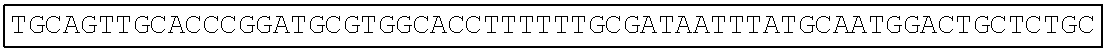 |
| | 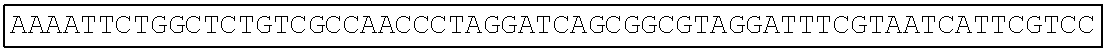 |
| | 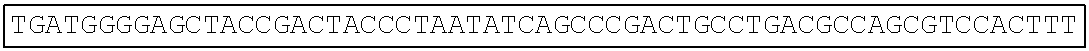 |
| | 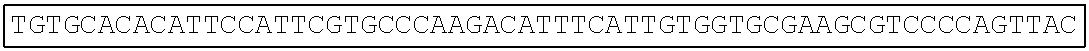 |
| | 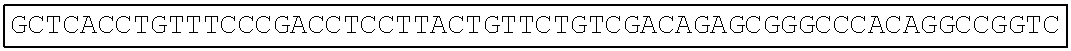 |
| | 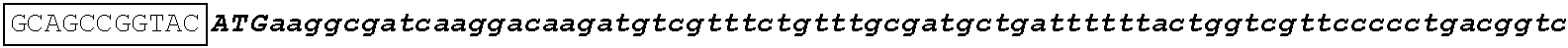 |
| | gcgttcggcacggactggtccctcagctacaaggcccgcaacgacatctttttctggaccgcgtttctcctgggctttttcaaggtccggacg |
| | taccgcatcggcgagcaggggtgctaccgtgacgggagctgcatctggctgtgcaaccaccgcgactggggcgactttttcatcgacctg |
| | tacgtcgtggagggccgtgcgtgctttctgtcccgcctgatggtgctgatcgcctttccagtgtttgggttctacgggctggtcacccgcaccg |
| | cggtgtttttccggcgcaacaccaagggccgcgatttcaccaagtttaaccaattcatctccggtcagatccagcgcagcccgtgccagg |
| | gcatcgtcgtgtacccggagggcacccgcaacatcaagcccgagtcgctgcccctgcgtcgcggcatgctgcgctacgcgtactccgag |
| | cgcaccccggtccagatcgtgatcaccacgaacaaggagcatgtgatctctcagaagatgcagcgagcgtccttcgggaccaagtgca |
| | tctcgggctttagcgaggtcatccggccgggcgactactccaacttcgaggacttctttgacgagatcaacaagacctggaaggccgagt |
| | ggaagcgcacctacggcgcgagcatctctaacgccgtggaggtgacgccttccgagatgaagctgtacatccccttctacaacattaag |
| | ctcggccagtacctgagcatgatggcctactcctacttcacggtcgccctgatggtgggagtggcctcgttcagctttgtgctgacccagtcc |
| | atgctgggcctgcgcgtggtccactteggegggttctccaccgactgcatcgtggtcagcatcttcctgggcgtcttcgtggcctgcaccaa |
| | gctcatcacccgaaagcaggacTAGcacgcccgcgcggcgcacctgacctgttctctcgagggcgcctgttctgccttgcgaaacaagc |
| | ccctggagcatgcgtgcatgatcgtctctggcgccccgccgcgcggtttgtcgccctcgcgggcgccgcggccgcgggggcgcattgaaa |
| | ttgttgcaaaccccacctgacagattgagggcccaggcaggaaggcgttgagatggaggtacaggagtcaagtaactgaaagtttttatgata |
| | actaacaacaaagggtcgtttctggccagcgaatgacaagaacaagattccacatttccgtgtagaggcttgccatcgaatgtgagcgggcgg |
| | gccgcggacccgacaaaacccttacgacgtggtaagaaaaacgtgggggcactgtccctgtagcctgaagaccagcaggagacgatcg |
| | gaagcatcacagcacaaccggtatcgatagcgtctgcgtgttgggagctggagtcgtgggcttgacgacggcgctgcagctcttgcag |
| | gatgtgcctggcgtgcgcgttcacgtcgtggctgagaaatatggcgacgaaacgttgacggctggggccggcgggctgtggatgcca |
| | tacgcattgggtacgcggccattggatgggattggtaggcttatggagggataatagagtttttgtcggatccaacgcatgttgatgca |
| | gtagcccggtgtgctgaaagtatggaaggatggtgcattggctattcacatgcgctgcccaccccttttcgcaggaaatgtgccggca |
| | tcgttggtgcaccgatggggaaaatcgacgttcgaccactacatgaaaatgtatacctctgaagatgcagcaactgcgggtgcgaaa |
| | cggataacggtttggtaggtgtatgtcgcagcatgtgctggattttgcgggcaactccccctgccacggcccattgcaggtgtcatgttg |
| | actggaggatacgaactttcggccgtcaaattcccagaggaggaccccctctgggccgacattgtgcccactgctcttccgcttgctgt |
| | ttcctgtgtgaaattgt |

The construct can be written as 5′DAO1b::CrTUB2:ScSUC2:PmPGH:CvNR:PmAMT03: ChprLPAAT2:PmPGH::3′DAO1b. Proceeding in the 5Y to 3Y direction bold, lowercase sequences represent genomic DNA from CHK22 that permit targeted integration at the DAO1b locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text that enables amplification of the ChprLPAAT2gene, followed by a linker, indicated by lowercase, bold italics, in which contains a C. vulgaris nitrate reductase 3′-UTR. The P. moriformis PmAMT03 promoter, indicated by boxed italicized text, drives the expression of the ChprLPAAT2. The initiator ATG and terminator CTAG codons of the ChprLPAAT2 are indicated by uppercase, bold italics, while the remainder of the gene is indicated by bold italics. The P. moriformis PmPGH 3′-UTR is indicated by lowercase underlined text followed by the CHK22 DAO1b genomic region indicated by bold, lowercase text.

Table 50 shows the fatty acid/TAG profiles of primary transformants of CHK22 transformed with pCHK98. The P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 50
Screen of primary transformants of CHK22 with pCHK980.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK980- | 0.04 | 0.02 | 21.31 | 2.97 | 61.56 | 10.33 | 0.64 | 0.27 | 0.1 | 1.44 |
| 1 | | | | | | | | | | |
| pCHK980- | 0.06 | 0.03 | 21.32 | 3.43 | 58.83 | 12.25 | 0.56 | 0.3 | 0.11 | 1.48 |
| 2 | | | | | | | | | | |
| pCHK980- | 0.06 | 0.03 | 21.18 | 3.42 | 58.51 | 12.63 | 0.53 | 0.3 | 0.11 | 1.51 |
| 3 | | | | | | | | | | |
| pCHK980- | 0.06 | 0.03 | 22.41 | 3.1 | 59.03 | 11.24 | 0.53 | 0.28 | 0.1 | 1.54 |
| 4 | | | | | | | | | | |
| pCHK980- | 0.06 | 0.03 | 21.29 | 3.32 | 58.8 | 12.37 | 0.55 | 0.3 | 0.11 | 1.43 |
| 5 | | | | | | | | | | |
| pCHK980- | 0.05 | 0.03 | 20.8 | 3.37 | 58.58 | 13.04 | 0.57 | 0.31 | 0.11 | 1.62 |
| 6 | | | | | | | | | | |
| CHK22 | 0.04 | 0.02 | 26.18 | 2.95 | 59.62 | 7.55 | 0.46 | 0.24 | 0.07 | 1.49 |
| Control | | | | | | | | | | |

### Example 25: Expressing Homo sapiens AGPAT1 in P. moriformis Strain CHK22

AGPAT1 was introduced into a P. moriformis strain CHK22. The expression construct pCHK966 contained 5′ and 3′ homology arms to permit targeted integration of AGPAT1 into the genome and is shown in Table 51.

TABLE 51
Integrative sequences for the transformation of P. moriformis with pCHK966
encoding AGPATI.

| SEQ | |
|---|---|
| ID | |
| NO: | Nucleotide Sequence |
| 37 | gctcttccgcttgcccgcaccctcgttgatctgggagccctgcgcagccccttaaatcatctcagtcaggtttctatgttcaactgagcct |
| | aaagggctttcgtcacgcgcacgagcacacgtatatcggctacgcagtctctcagaagcggtagaacagttcgcaagccctcgtcgg |
| | tcgaaaacttgcgccagtactattgaattaaattaaatgatcgaatgagacgcgaaacttttgcagaatgccactgagtttgcccaga |
| | gaatgggagtggcgccattcaccatccgcctgtgcacggcctgattcgccgagacgatgaatggcgagaccagggagcggcttgcg |
| | agccccgagccggtagcaggaataacggtcgacaatcttcctgtccaattactggcaaccattagaaagagccggagcgcgttgaa |
| | attctgcaatcgagtaatttttcgatgcggcgggcctgctgaaccctaaggctccggcctatgtttaaggcgatccaagatgcacgcgg |
| | ccccaggcacgtgtctcaagcacaaaccccagccttagtttcgagactttgggagatagcggcagatatctagtttggcattttgtatat |
| | taattaagtctcgcaatggagcgctctgatgcggtgcagcgtcggctgcagcacctggcagtggcgctggggtegccctatcgctcgg |
| | 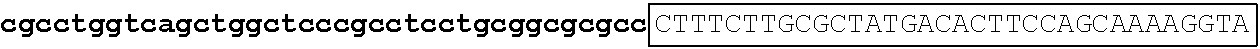 |
| | 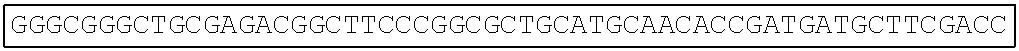 |
| | 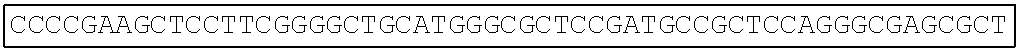 |
| | 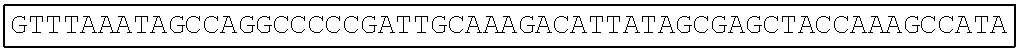 |
| | 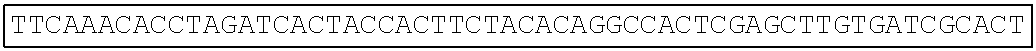 |
| | 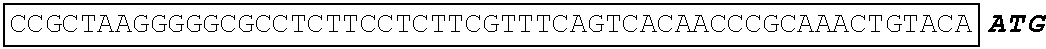 |
| | ctgctgcaggccttcctgttcctgctggccggcttcgccgccaagatcagcgcctccatgacgaacgagacgtccgaccgccccctggtg |
| | cacttcacccccaacaagggctggatgaacgaccccaacggcctgtggtacgacgagaaggacgccaagtggcacctgtacttccagt |
| | acaacccgaacgacaccgtctgggggacgcccttgttctggggccacgccacgtccgacgacctgaccaactgggaggaccagccca |
| | tcgccatcgccccgaagcgcaacgactccggcgccttctccggctccatggtggtggactacaacaacacctccggcttcttcaacgaca |
| | ccatcgacccgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctggtacgagc |
| | cctcccagaagtggatcatgaccgcggccaagtcccaggactacaagatcgagatctactcctccgacgacctgaagtcctggaagctg |
| | gagtccgcgttcgccaacgagggcttcctcggctaccagtacgagtgccccggcctgatcgaggtccccaccgagcaggaccccagca |
| | agtcctactgggtgatgttcatctccatcaaccccggcgccccggccggcggctccttcaaccagtacttcgtcggcagcttcaacggcac |
| | ccacttcgaggccttcgacaaccagtcccgcgtggtggacttcggcaaggactactacgccctgcagaccttcttcaacaccgacccgac |
| | ctacgggagcgccctgggcatcgcgtgggcctccaactgggagtactccgccttcgtgcccaccaacccctggcgctcctccatgtccctc |
| | gtgcgcaagttctccctcaacaccgagtaccaggccaacccggagacggagctgatcaacctgaaggccgagccgatcctgaacatc |
| | agcaacgccggcccctggagccggttcgccaccaacaccacgttgacgaaggccaacagctacaacgtcgacctgtccaacagcacc |
| | ggcaccctggagttcgagctggtgtacgccgtcaacaccacccagacgatctccaagtccgtgttcgcggacctctccctctggttcaagg |
| | gcctggaggaccccgaggagtacctccgcatgggcttcgaggtgtccgcgtcctccttcttcctggaccgcgggaacagcaaggtgaag |
| | ttcgtgaaggagaacccctacttcaccaaccgcatgagcgtgaacaaccagcccttcaagagcgagaacgacctgtcctactacaagg |
| | tgtacggcttgctggaccagaacatcctggagctgtacttcaacgacggcgacgtcgtgtccaccaacacctacttcatgaccaccggga |
| | acgccctgggctccgtgaacatgacgactggacaacctgttctacatcgacaagttccaggtgcgcgaggtcaagtgactTA |
| | AGACGCCCGCGCGGCGCACCTGACCTGTTCTCTCGAGGGCGCCTGTTCTGCCTTGCGA |
| | AACAAGCCCCTGGAGCATGCGTGCATGATCGTCTCTGGCGCCCCGCCGCGCGGTTTGT |
| | CGCCCTCGCGGGCGCCGCGGCCGCGGGGGCGCATTGAAATTGTTGCAAACCCCACCT |
| | GACAGATTGAGGGCCCAGGCAGGAAGGCGTTGAGATGGAGGTACAGGAGTCAAGTA |
| | ACTGAAAGTTTTTATGATAACTAACAACAAAGGGTCGTTTCTGGCCAGCGAATGACA |
| | AGAACAAGATTCCACATTTCCGTGTAGAGGCTTGCCATCGAATGTGAGCGGGCGGGC |
| | CGCGGACCCGACAAAACCCTTACGACGTGGTAAGAAAAACGTGGCGGGCACTGTCCC |
| | TGTAGCCTGAAGACCAGCAGGAGACGATCGGAAGCATCACAGCACAACCGGtgcagcag |
| | cagctcggatagtatcgacacactctggacgctggtcgtgtgatggactgttgccgccacacttgctgccttgacctgtgaatatccctgcc |
| | gcttttatcaaacagcctcagtgtgtttgatcttgtgtgtacgcgcttttgcgagttgctagctgcttgtgctatttgcgaataccacccccagca |
| | tccccttccctcgtttcatatcgcttgcatcccaaccgcaacttatctacgctgtcctgctatccctcagegctgctcctgctcctgctcactgcc |
| | cctcgcacagccttggtttgggctccgcctgtattctcctggtactgcaacctgtaaaccagcactgcaatgctgatgcacgggaagtagtg |
| | 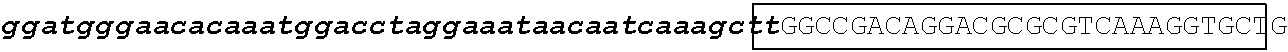 |
| | 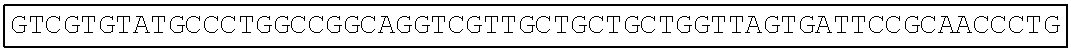 |
| | 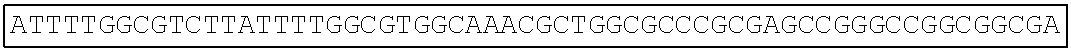 |
| | 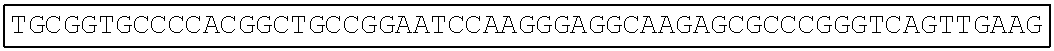 |
| | 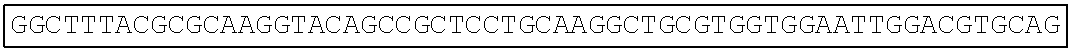 |
| | 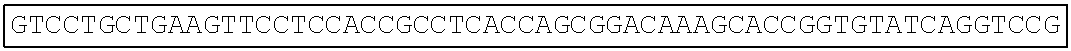 |
| | 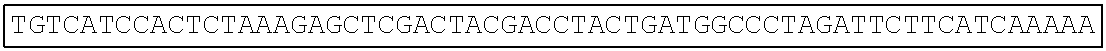 |
| | 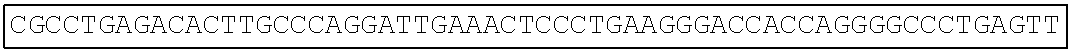 |
| | 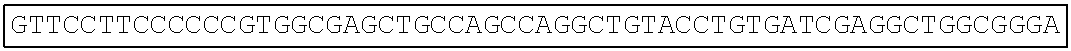 |
| | 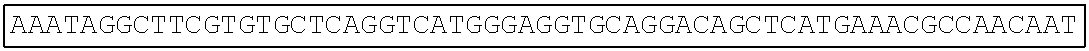 |
| | 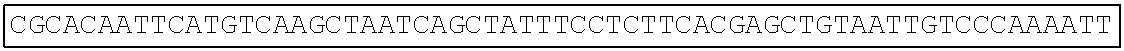 |
| | 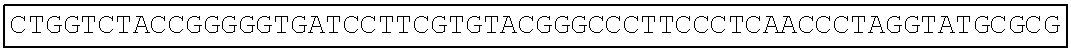 |
| | 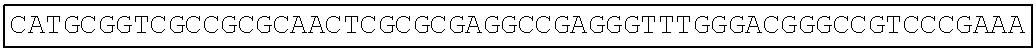 |
| | 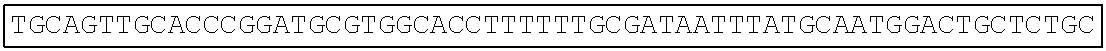 |
| | 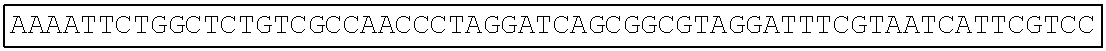 |
| | 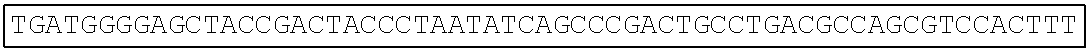 |
| | 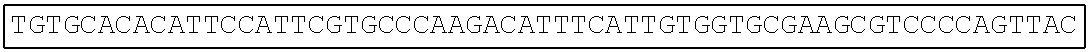 |
| | 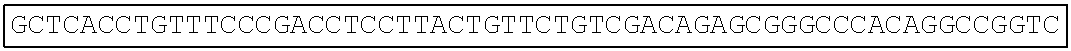 |
| | 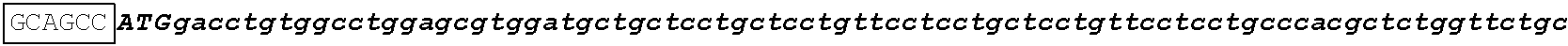 |
| | tccccgteggctaagtacttctttaagatggccttctacaacggctggattctgttcctcgccgtcctggcgatccctgtgtgtgccgtgcggg |
| | gccgcaacgtggagaacatgaagatcctgcgcctcatgctcctgcatatcaagtacctctacggcatccgtgtggaggtccgcggggcg |
| | catcacttccegccctcccagccctacgtegtggtctccaaccaccagtegagcctggacctcctggggatgatggaggtcctgccggga |
| | cgctgcgtccctattgcgaagcgcgaactcctgtgggcgggctcggctggcctggcttgttggctggcgggcgtgatctttatcgaccgca |
| | agcgcacgggcgacgccattagcgtgatgtccgaggtggcccagacgctcctgacgcaggacgtccgggtgtgggtcttcccggaggg |
| | cacccgcaaccacaacgggagcatgctgcccttcaagcgcggcgcgttccacctggcggtgcaggctcaggtccccategtcccaatc |
| | gtgatgtcctcgtaccaggacttctactgcaagaaggagcgacgctttacctctggccaatgccaggtgcgagtectcccgcccgtgccg |
| | acggagggcctcacccccgatgacgtcccggcgctggccgaccgagtccgacactccatgctgaccgtgttccgggagatcagcaccg |
| | atggccgcggcggaggcgactacctgaagaagccgggeggcggcggcTAGacgcccgcgcggcgcacctgacctgttctctcgag |
| | ggcgcctgttctgccttgcgaaacaagcccctggagcatgcgtgcatgatcgtctctggcgccccgccgcgcggtttgtcgccctcgcgggc |
| | gccgcggccgcgggggcgcattgaaattgttgcaaaccccacctgacagattgagggcccaggcaggaaggcgttgagatggaggtaca |
| | ggagtcaagtaactgaaagtttttatgataactaacaacaaagggtcgtttctggccagcgaatgacaagaacaagattccacatttccgtgtag |
| | aggcttgccatcgaatgtgagcggggggccgcggacccgacaaaacccttacgacgtggtaagaaaaacgtgggggcactgtccctgt |
| | agcctgaagaccagcaggagacgatcggaagcatcacagcacaaccggtatcgatagcgtctgcgtgttgggagctggagtcgtgggc |
| | ttgacgacggcgctgcagctcttgcaggatgtgcctggcgtgcgcgttcacgtcgtggctgagaaatatggcgacgaaacgttgacg |
| | gctggggccggcgggctgtggatgccatacgcattgggtacgcggccattggatgggattggtaggcttatggagggataatagagt |
| | ttttgtcggatccaacgcatgttgatgcagtagcccggtgtgctgaaagtatggaaggatggtgcattggctattcacatgegctgccc |
| | accccttttcgcaggaaatgtgccggcatcgttggtgcaccgatggggaaaatcgacgttcgaccactacatgaaaatgtatacctct |
| | gaagatgcagcaactgcgggtgcgaaacggataacggtttggtaggtgtatgtcgcagcatgtgctggattttgcgggcaactcccc |
| | ctgccacggcccattgcaggtgtcatgttgactggaggatacgaacttteggccgtcaaattcccagaggaggaccccctctgggccg |
| | acattgtgcc cactgctcttccgcttgctgtttcctgtgtgaaattgt |

The construct can be written as 5′DAO1b::CrTUB2:ScSUC2:PmPGH:CvNR:PmAMT03:AGPAT1:PmPGH::3′DAO1b. Proceeding in the 5′ to 3′ direction bold, lowercase sequences represent genomic DNA from CHK22 that permit targeted integration at the DAO1b locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text that enables amplification of the AGPAT1 gene, followed by a linker, indicated by lowercase, bold italics, in which contains a C. vulgaris nitrate reductase 3′-UTR. The P. moriformis PmAMT03 promoter, indicated by boxed italicized text, drives the expression of the AGPAT1. The initiator ATG and terminator TAG codons of the AGPAT1 are indicated by uppercase, bold italics, while the remainder of the gene is indicated by bold italics. The P. moriformis PmPGH 3′-UTR is indicated by lowercase underlined text followed by the CHK22 DAO1b genomic region indicated by bold, lowercase text.

Table 52 shows the fatty acid/TAG profiles of primary transformants of CHK22 transformed with pCHK966. The P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 52
Screen of primary transformants of CHK22 with pCHK966.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK966- | 0.05 | 1.54 | 20.69 | 3.16 | 59.29 | 12.79 | 0.58 | 0.32 | 0.00 | 1.58 |
| 1 | | | | | | | | | | |
| pCHK966- | 0.05 | 1.51 | 20.60 | 3.66 | 57.62 | 14.29 | 0.54 | 0.23 | 0.12 | 3.6 |
| 2 | | | | | | | | | | |
| pCHK966- | 0.11 | 2.16 | 20.07 | 2.85 | 53.51 | 18.23 | 0.78 | 0.14 | 0.00 | 3.49 |
| 3 | | | | | | | | | | |
| pCHK966- | 0.04 | 1.50 | 23.85 | 3.07 | 59.39 | 9.80 | 0.52 | 0.31 | 0.09 | 1.59 |
| 4 | | | | | | | | | | |
| pCHK966- | 0.06 | 1.60 | 20.73 | 3.14 | 58.41 | 13.59 | 0.60 | 0.30 | 0.11 | 1.53 |
| 5 | | | | | | | | | | |
| pCHK966- | 0.06 | 1.66 | 20.29 | 3.34 | 56.54 | 15.71 | 0.62 | 0.22 | 0.12 | 3.22 |
| 6 | | | | | | | | | | |
| pCHK966- | 0.04 | 1.43 | 25.51 | 2.69 | 59.72 | 8.47 | 0.48 | 0.23 | 0.08 | 2.73 |
| 7 | | | | | | | | | | |
| pCHK966- | 0.05 | 1.54 | 22.18 | 3.26 | 57.83 | 12.86 | 0.55 | 0.24 | 0.00 | 2.77 |
| 8 | | | | | | | | | | |
| CHK22 | 0.04 | 1.50 | 26.06 | 3.13 | 59.36 | 7.76 | 0.46 | 0.32 | 0.00 | 1.58 |
| Control | | | | | | | | | | |

### Example 26: Expressing Chlamydomonas eustigma LPAAT2 (CeLPAAT2) in P. moriformis Strain CHK22

CeLPAAT2 was introduced into a P. moriformis strain CHK22. The expression construct pCHK970 contained 5Y and 3Y homology arms to permit targeted integration of CeLPAAT2 into the genome and is shown in Table 53.

TABLE 53
Integrative sequences for the transformation of P. moriformis with pCHK970
encoding CeLPAAT2.

| SEQ | |
|---|---|
| ID | |
| NO: | Nucleotide Sequence |
| 38 | gctcttccgcttgcccgcaccctcgttgatctgggagccctgcgcagccccttaaatcatctcagtcaggtttctatgttcaactgagcct |
| | aaagggctttcgtcacgcgcacgagcacacgtatatcggctacgcagtctctcagaagcggtagaacagttcgcaagccctcgtcgg |
| | tcgaaaacttgcgccagtactattgaattaaattaaatgatcgaatgagacgcgaaacttttgcagaatgccactgagtttgcccaga |
| | gaatgggagtggcgccattcaccatccgcctgtgcacggcctgattcgccgagacgatgaatggcgagaccagggagcggcttgcg |
| | agccccgagccggtagcaggaataacggtcgacaatcttcctgtccaattactggcaaccattagaaagagccggagcgcgttgaa |
| | attctgcaatcgagtaatttttcgatgcggcgggcctgctgaaccctaaggctccggcctatgtttaaggcgatccaagatgcacgcgg |
| | ccccaggcacgtgtctcaagcacaaaccccagccttagtttcgagactttgggagatagcggcagatatctagtttggcattttgtatat |
| | taattaagtctcgcaatggagcgctctgatgcggtgcagcgtcggctgcagcacctggcagtggcgctggggtcgccctatcgctcgg |
| | 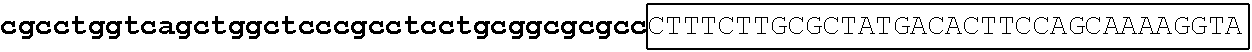 |
| | 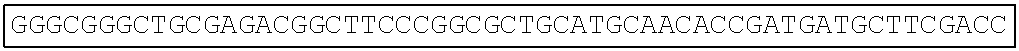 |
| | 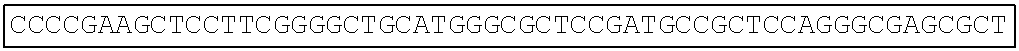 |
| | 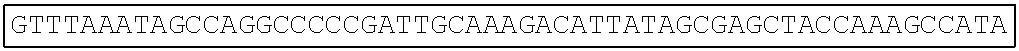 |
| | 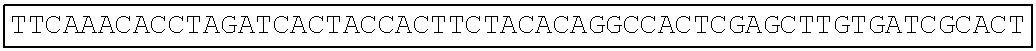 |
| | 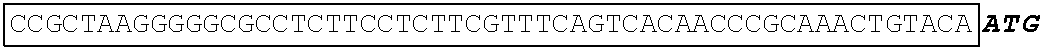 |
| | ctgctgcaggccttcctgttcctgctggccggcttcgccgccaagatcagcgcctccatgacgaacgagacgtccgaccgccccctggtg |
| | cacttcacccccaacaagggctggatgaacgaccccaacggcctgtggtacgacgagaaggacgccaagtggcacctgtacttccagt |
| | acaacccgaacgacaccgtctgggggacgcccttgttctggggccacgccacgtccgacgacctgaccaactgggaggaccagccca |
| | tcgccatcgccccgaagcgcaacgactccggcgccttctccggctccatggtggtggactacaacaacacctccggcttcttcaacgaca |
| | ccatcgacccgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctggtacgagc |
| | cctcccagaagtggatcatgaccgcggccaagtcccaggactacaagatcgagatctactcctccgacgacctgaagtcctggaagctg |
| | gagtccgcgttcgccaacgagggcttcctcggctaccagtacgagtgccccggcctgatcgaggtccccaccgagcaggaccccagca |
| | agtcctactgggtgatgttcatctccatcaaccccggcgccccggccggcggctccttcaaccagtacttcgtcggcagcttcaacggcac |
| | ccacttcgaggccttcgacaaccagtcccgcgtggtggacttcggcaaggactactacgccctgcagaccttcttcaacaccgacccgac |
| | ctacgggagcgccctgggcatcgcgtgggcctccaactgggagtactccgccttcgtgcccaccaacccctggcgctcctccatgtccctc |
| | gtgcgcaagttctccctcaacaccgagtaccaggccaacccggagacggagctgatcaacctgaaggccgagccgatcctgaacatc |
| | agcaacgccggcccctggagccggttcgccaccaacaccacgttgacgaaggccaacagctacaacgtcgacctgtccaacagcacc |
| | ggcaccctggagttcgagctggtgtacgccgtcaacaccacccagacgatctccaagtccgtgttcgcggacctctccctctggttcaagg |
| | gcctggaggaccccgaggagtacctccgcatgggcttcgaggtgtccgcgtcctccttcttcctggaccgcgggaacagcaaggtgaag |
| | ttcgtgaaggagaacccctacttcaccaaccgcatgagcgtgaacaaccagcccttcaagagcgagaacgacctgtcctactacaagg |
| | tgtacggcttgctggaccagaacatcctggagctgtacttcaacgacggcgacgtcgtgtccaccaacacctacttcatgaccaccggga |
| | acgccctgggctccgtgaacatgacgacgggggtggacaacctgttctacatcgacaagttccaggtgcgcgaggtcaagtgactTA |
| | AGACGCCCGCGCGGCGCACCTGACCTGTTCTCTCGAGGGCGCCTGTTCTGCCTTGCGA |
| | AACAAGCCCCTGGAGCATGCGTGCATGATCGTCTCTGGCGCCCCGCCGCGCGGTTTGT |
| | CGCCCTCGCGGGCGCCGCGGCCGCGGGGGCGCATTGAAATTGTTGCAAACCCCACCT |
| | GACAGATTGAGGGCCCAGGCAGGAAGGCGTTGAGATGGAGGTACAGGAGTCAAGTA |
| | ACTGAAAGTTTTTATGATAACTAACAACAAAGGGTCGTTTCTGGCCAGCGAATGACA |
| | AGAACAAGATTCCACATTTCCGTGTAGAGGCTTGCCATCGAATGTGAGCGGGCGGGC |
| | CGCGGACCCGACAAAACCCTTACGACGTGGTAAGAAAAACGTGGCGGGCACTGTCCC |
| | TGTAGCCTGAAGACCAGCAGGAGACGATCGGAAGCATCACAGCACAACCGGtgcagcag |
| | cagctcggatagtatcgacacactctggacgctggtgtgtgatggactgttgccgccacacttgctgccttgacctgtgaatatccctgcc |
| | gcttttatcaaacagcctcagtgtgtttgatcttgtgtgtacgcgcttttgcgagttgctagctgcttgtgctatttgcgaataccacccccagca |
| | tccccttccctegtttcatatcgcttgcatcccaaccgcaacttatctacgctgtcctgctatccctcagegctgctcctgctcctgctcactgcc |
| | cctcgcacagccttggtttgggctccgcctgtattctcctggtactgcaacctgtaaaccagcactgcaatgctgatgcacgggaagtagtg |
| | 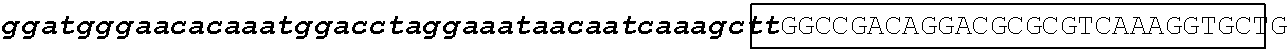 |
| | 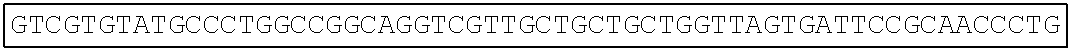 |
| | 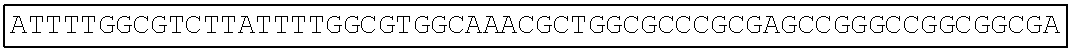 |
| | 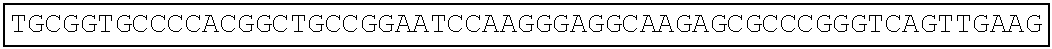 |
| | 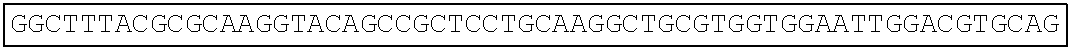 |
| | 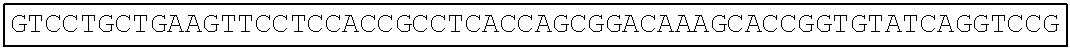 |
| | 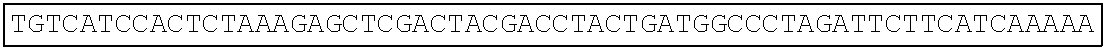 |
| | 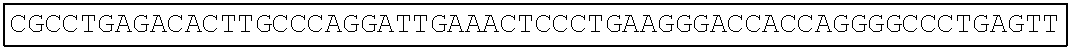 |
| | 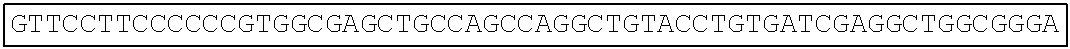 |
| | 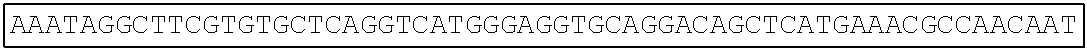 |
| | 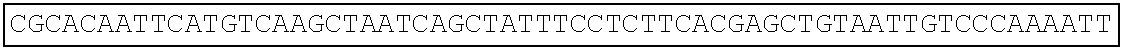 |
| | 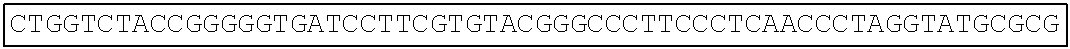 |
| | 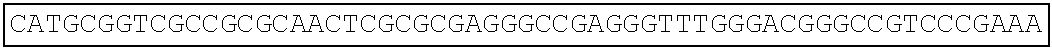 |
| | 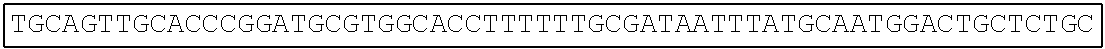 |
| | 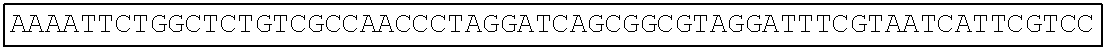 |
| | 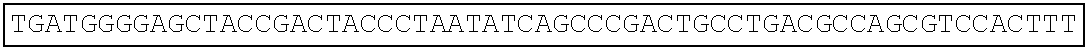 |
| | 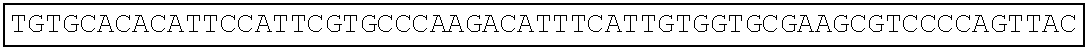 |
| | 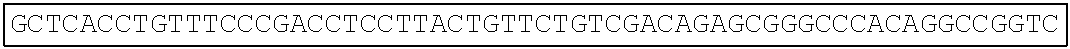 |
| | 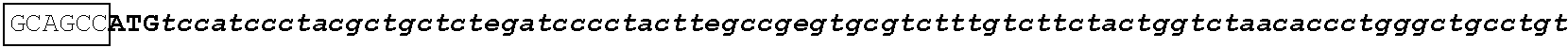 |
| | gctacaagatcaagttcatgaacctgatcggcccccgcaaggacatgcatgactggtcggagtttatgaactggtttttcggcgtccagtat |
| | cggcaggtcggggagcagtcgctgcacaagggtgagcgctgcctgtacctgtccaaccatcggtcctgggcggactttttcgtcgacatct |
| | acctgacggagggcatggcggcccccatgagccggaacctggtgttctacgtctttccatttttcatgacctcggtgtccatcctcaagggc |
| | attatcctctttaagcgtggggtcatcgaggacaagcagaagtttaacgcgtggctcgactegaagctegactcttccatccgcccctecct |
| | cctggtctaccctgagggccaccgcagcaccegegegtegagcctccccctcaagcgcggcatgctgcactacgcgcactcccgcaag |
| | ctccctatccagattatcatgacgaagggcaaggagggtgtgctcagcgagaagcacatgcgcgcctcttacggcgtgaccctggcgac |
| | gggattctcggaggtgatcaagtcgtccaacttccctgagttcgaggcgttcgccctgaaggtgcaacaggtctgggacgcgaagtggca |
| | ggaggtctacaccgccgacatctctegacggcctgactgcTGAacgcccgcgcggcgcacctgacctgttctctcgagggcgcctgttct |
| | gccttgcgaaacaagcccctggagcatgcgtgcatgatcgtctctggegccccgccgcgcggtttgtcgccctcgcgggcgccgcggccg |
| | cgggggcgcattgaaattgttgcaaaccccacctgacagattgagggcccaggcaggaaggcgttgagatggaggtacaggagtcaagta |
| | actgaaagtttttatgataacgggtcgtttctggccagcgaatgacaagaacaagattccacatttccgtgtagaggcttgccatc |
| | gaatgtgagcgggcgggccgcggacccgacaaaacccttacgacgtggtaagaaaaacgtgggggcactgtccctgtagcctgaagac |
| | cagcaggagacgatcggaagcatcacagcacaaccggtatcgatagcgtctgcgtgttgggagctggagtcgtgggcttgacgacggc |
| | gctgcagctcttgcaggatgtgcctggcgtgcgcgttcacgtcgtggctgagaaatatggcgacgaaacgttgacggctggggccgg |
| | cgggctgtggatgccatacgcattgggtacgcggccattggatgggattggtaggcttatggagggataatagagtttttgtcggatcc |
| | aacgcatgttgatgcagtagcccggtgtgctgaaagtatggaaggatggtgcattggctattcacatgcgctgcccaccccttttcgca |
| | ggaaatgtgccggcatcgttggtgcaccgatggggaaaatcgacgttcgaccactacatgaaaatgtatacctctgaagatgcagca |
| | actgcgggtgcgaaacggataacggtttggtaggtgtatgtcgcagcatgtgctggattttgcgggcaactccccctgccacggccca |
| | ttgcaggtgtcatgttgactggaggatacgaactttcggccgtcaaattcccagaggaggaccccctctgggccgacattgtgcccact |
| | gctcttccgcttgctgtttcctgtgtgaaattgt |

The construct can be written as 5′DAO1b::CrTUB2:ScSUC2:PmPGH:CvNR:PmAMT03:CeLPAAT2:PmPGH::3′DAO1b. Proceeding in the 5′ to 3′ direction bold, lowercase sequences represent genomic DNA from CHK22 that permit targeted integration at the DAO1b locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text that enables amplification of the CeLPAAT2 gene, followed by a linker, indicated by lowercase, bold italics, in which contains a C. vulgaris nitrate reductase 3′-UTR. The P. moriformis PmAMT03 promoter, indicated by boxed italicized text, drives the expression of the CeLPAAT2. The initiator ATG and terminator TAG codons of the CeLPAAT2 are indicated by uppercase, bold italics, while the remainder of the gene is indicated by bold italics. The P. moriformis PmPGH 3′-UTR is indicated by lowercase underlined text followed by the CHK22 DAO1b genomic region indicated by bold, lowercase text.

Table 54 shows the fatty acid/TAG profiles of primary transformants of CHK22 transformed with pCHK970. The P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 54
Screen of primary transformants of CHK22 with pCHK970.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK970- | 0.05 | 1.50 | 21.40 | 3.08 | 59.49 | 12.02 | 0.57 | 0.32 | 0.11 | 1.58 |
| 1 | | | | | | | | | | |
| pCHK970- | 0.05 | 1.55 | 22.86 | 3.06 | 58.95 | 11.22 | 0.54 | 0.31 | 0.00 | 1.59 |
| 2 | | | | | | | | | | |
| pCHK970- | 0.06 | 1.62 | 20.68 | 3.30 | 58.71 | 13.22 | 0.59 | 0.32 | 0.11 | 1.5 |
| 3 | | | | | | | | | | |
| pCHK970- | 0.06 | 1.63 | 20.85 | 3.07 | 58.65 | 13.27 | 0.60 | 0.31 | 0.11 | 1.59 |
| 4 | | | | | | | | | | |
| pCHK970- | 0.07 | 1.90 | 24.20 | 2.98 | 54.59 | 13.64 | 0.66 | 0.29 | 0.09 | 1.59 |
| 5 | | | | | | | | | | |
| pCHK970- | 0.05 | 1.58 | 22.46 | 3.12 | 58.84 | 11.58 | 0.55 | 0.32 | 0.10 | 1.57 |
| 6 | | | | | | | | | | |
| pCHK970- | 0.04 | 1.55 | 24.70 | 3.03 | 59.21 | 9.23 | 0.52 | 0.30 | 0.09 | 1.54 |
| 7 | | | | | | | | | | |
| pCHK970- | 0.06 | 1.58 | 20.53 | 3.24 | 58.59 | 13.56 | 0.61 | 0.32 | 0.00 | 1.55 |
| 8 | | | | | | | | | | |
| CHK22 | 0.04 | 1.50 | 26.06 | 3.13 | 59.36 | 7.76 | 0.46 | 0.32 | 0.00 | 1.58 |
| Control | | | | | | | | | | |

### Example 27: Expressing Pedinophyceae sp. LPAAT2 (PedLPAAT2) in P. moriformis Strain CHK22 Using the PmAMT03 Promoter

PedLPAAT2 was introduced into a P. moriformis strain CHK22. The expression construct pCHK972 contained 5Y and 3Y homology arms to permit targeted integration of PedLPAAT2 into the genome and is shown in Table 55.

TABLE 55
Integrative sequences for the transformation of P. moriformis with pCHK972
encoding PedLPAAT2.

| SEQ | |
|---|---|
| ID | |
| NO: | Nucleotide Sequence |
| 39 | gctcttccgcttgcccgcaccctcgttgatctgggagccctgcgcagccccttaaatcatctcagtcaggtttctatgttcaactgagcct |
| | aaagggctttcgtcacgcgcacgagcacacgtatatcggctacgcagtctctcagaagcggtagaacagttcgcaagccctcgtcgg |
| | tcgaaaacttgcgccagtactattgaattaaattaaatgatcgaatgagacgcgaaacttttgcagaatgccactgagtttgcccaga |
| | gaatgggagtggcgccattcaccatccgcctgtgcacggcctgattcgccgagacgatgaatggcgagaccagggagcggcttgcg |
| | agccccgagccggtagcaggaataacggtcgacaatcttcctgtccaattactggcaaccattagaaagagccggagcgcgttgaa |
| | attctgcaatcgagtaatttttcgatgcggcgggcctgctgaaccctaaggctccggcctatgtttaaggcgatccaagatgcacgcgg |
| | ccccaggcacgtgtctcaagcacaaaccccagccttagtttcgagactttgggagatagcggcagatatctagtttggcattttgtatat |
| | taattaagtctcgcaatggagcgctctgatgcggtgcagcgtcggctgcagcacctggcagtggcgctggggtcgccctatcgctcgg |
| | cgcctggtcagctggctcccgcctcctgeggcgcgcc 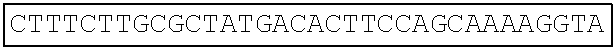 |
| | 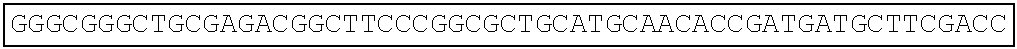 |
| | 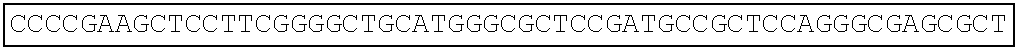 |
| | 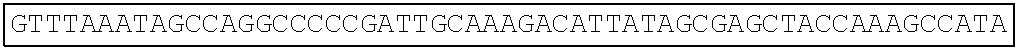 |
| | 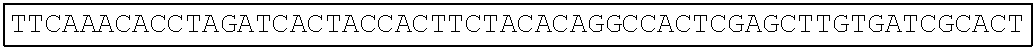 |
| | 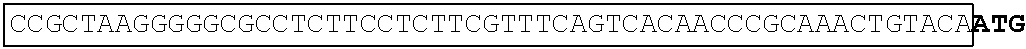 |
| | ctgctgcaggccttcctgttcctgctggccggcttcgccgccaagatcagcgcctccatgacgaacgagacgtccgaccgccccctggtg |
| | cacttcacccccaacaagggctggatgaacgaccccaacggcctgtggtacgacgagaaggacgccaagtggcacctgtacttccagt |
| | acaacccgaacgacaccgtctgggggacgcccttgttctggggccacgccacgtccgacgacctgaccaactgggaggaccagccca |
| | tcgccatcgccccgaagcgcaacgactccggcgccttctccggctccatggtggtggactacaacaacacctccggcttcttcaacgaca |
| | ccatcgacccgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctggtacgagc |
| | cctcccagaagtggatcatgaccgcggccaagtcccaggactacaagatcgagatctactcctccgacgacctgaagtcctggaagctg |
| | gagtccgcgttcgccaacgagggcttcctcggctaccagtacgagtgccccggcctgatcgaggtccccaccgagcaggaccccagca |
| | agtcctactgggtgatgttcatctccatcaaccccggcgccccggccggcggctccttcaaccagtacttcgtcggcagcttcaacggcac |
| | ccacttcgaggccttcgacaaccagtcccgcgtggtggacttcggcaaggactactacgccctgcagaccttcttcaacaccgacccgac |
| | ctacgggagcgccctgggcatcgcgtgggcctccaactgggagtactccgccttcgtgcccaccaacccctggcgctcctccatgtccctc |
| | gtgcgcaagttctccctcaacaccgagtaccaggccaacccggagacggagctgatcaacctgaaggccgagccgatcctgaacatc |
| | agcaacgccggcccctggagccggttcgccaccaacaccacgttgacgaaggccaacagctacaacgtcgacctgtccaacagcacc |
| | ggcaccctggagttcgagctggtgtacgccgtcaacaccacccagacgatctccaagtccgtgttcgcggacctctccctctggttcaagg |
| | gcctggaggaccccgaggagtacctccgcatgggcttcgaggtgtccgcgtcctccttcttcctggaccgcgggaacagcaaggtgaag |
| | ttcgtgaaggagaacccctacttcaccaaccgcatgagcgtgaacaaccagcccttcaagagcgagaacgacctgtcctactacaagg |
| | tgtacggcttgctggaccagaacatcctggagctgtacttcaacgacggcgacgtcgtgtccaccaacacctacttcatgaccaccggga |
| | acgccctgggctccgtgaacatgacgacgggggtggacaacctgttctacatcgacaagttccaggtgcgcgaggtcaagtgactTA |
| | AGACGCCCGCGCGGCGCACCTGACCTGTTCTCTCGAGGGCGCCTGTTCTGCCTTGCGA |
| | AACAAGCCCCTGGAGCATGCGTGCATGATCGTCTCTGGCGCCCCGCCGCGCGGTTTGT |
| | CGCCCTCGCGGGCGCCGCGGCCGCGGGGGCGCATTGAAATTGTTGCAAACCCCACCT |
| | GACAGATTGAGGGCCCAGGCAGGAAGGCGTTGAGATGGAGGTACAGGAGTCAAGTA |
| | ACTGAAAGTTTTTATGATAACTAACAACAAAGGGTCGTTTCTGGCCAGCGAATGACA |
| | AGAACAAGATTCCACATTTCCGTGTAGAGGCTTGCCATCGAATGTGAGCGGGCGGGC |
| | CGCGGACCCGACAAAACCCTTACGACGTGGTAAGAAAAACGTGGCGGGCACTGTCCC |
| | TGTAGCCTGAAGACCAGCAGGAGACGATCGGAAGCATCACAGCACAACCGGtgcagcag |
| | cagctcggatagtatcgacacactctggacgctggtegtgtgatggactgttgccgccacacttgctgccttgacctgtgaatatccctgcc |
| | gcttttatcaaacagcctcagtgtgtttgatcttgtgtgtacgcgcttttgcgagttgctagctgcttgtgctatttgcgaataccacccccagca |
| | tccccttccctcgtttcatategcttgcatcccaaccgcaacttatctacgctgtcctgctatccctcagegctgctcctgctcctgctcactgcc |
| | cctcgcacagccttggtttgggctccgcctgtattctcctggtactgcaacctgtaaaccagcactgcaatgctgatgcacgggaagtagtg |
| | ggatgggaacacaaatggacctaggaaataacaatcaaagctt 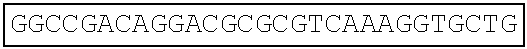 |
| | 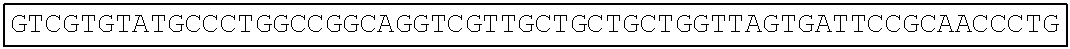 |
| | 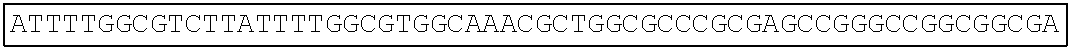 |
| | 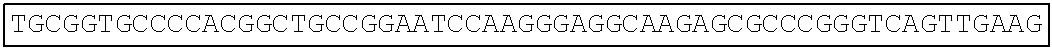 |
| | 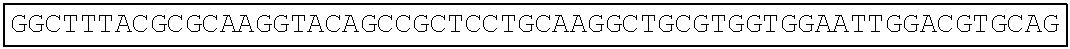 |
| | 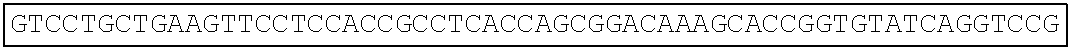 |
| | 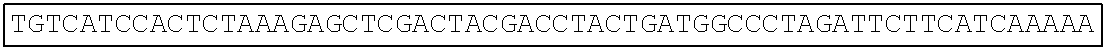 |
| | 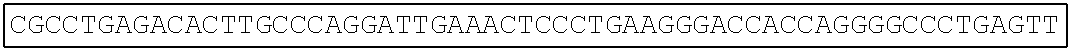 |
| | 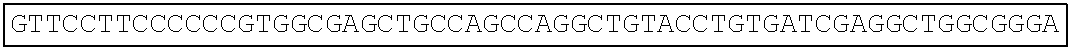 |
| | 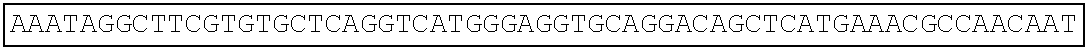 |
| | 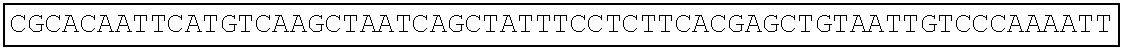 |
| | 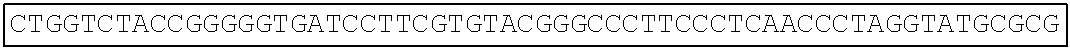 |
| | 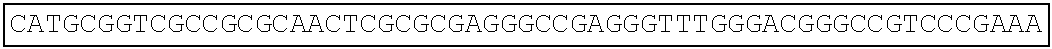 |
| | 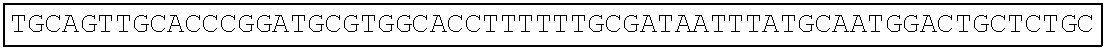 |
| | 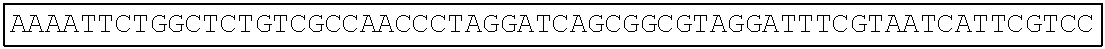 |
| | 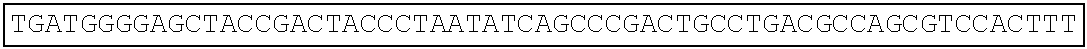 |
| | 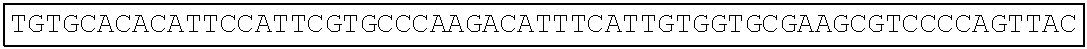 |
| | 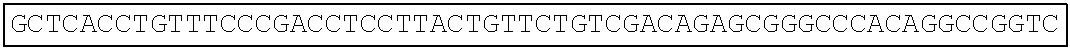 |
| | 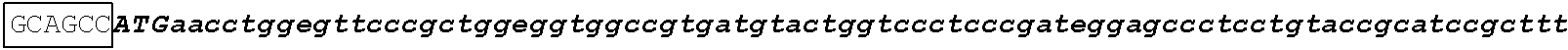 |
| | ctcaacgtgttcggcccccgtaacgatatgctggagtgggcggagatgatgcgtgccttcctcggagtgcgcttcaagctcgtgggccaa |
| | cgtcgcctgcacaagggcgagcgtatggtgtacctcgccaaccaccgctcgtgggccgactttttcctggacgtgtggacgaccgaggc |
| | gcgctgctcgatcatgtcccgctggatggtcttctttgcctttccggtgtttatggccagcgtcategtcctccgcggcatcgtgctgttcaagc |
| | ggggcaagatcaaggacaaggacgcgtttaacgcctggctggacaagaccctggaccgccaggtcaactccggctctctgctcctgta |
| | cccggaggggacgcgtaacacgaagcctcatgcgctgcctctgaagcgcgggatgctgaagtactcctggagccgeggcctgccattc |
| | caaattatcgtcacgaccaacaaggagcgcgtcctgtcggagaagaagatggcggtcaagttcggggtgacgtgctgtacggggttttc |
| | cgaggtcatcgagtccaagcgctacaaggatgacttcgacggctttatggaggaggtccagcgcgtgtgggataaggagtgggcggag |
| | gtgtacaaggccgctgcggagcccgagaagctggaggacctcacgatccccagcccggacgggttcgactacctcccctcgatgaag |
| | ctgctccaggtgctggtgaccctcctgaccatcgcggtgttcagcgcgatggtcgtgggctccgcctacgccctgggtctggtggtccccgc |
| | ggcccaggacgcctggaagtacgcgagctacgtcctggcgtcgatgcccatcctgggcctcgccagcaagctgctcgggggcagcaa |
| | ggtcggcgctatcaagtatccgggcctcgccagcaagaagattgacTGAacgcccgcgcggcgcacctgacctgttctctcgagggcg |
| | cctgttctgccttgcgaaacaagcccctggagcatgcgtgcatgatcgtctctggcgcccgccgcgcggtttgtcgccctcgcgggcgccg |
| | cggccgcgggggcgcattgaaattgttgcaaaccccacctgacagattgagggcccaggcaggaaggcgttgagatggaggtacaggagt |
| | caagtaactgaaagtttttatgataactaacaacaaagggtcgtttctggccagcgaatgacaagaacaagattccacatttccgtgtagaggctt |
| | gccatcgaatgtgagcggggggccgcggacccgacaaaacccttacgacgtggtaagaaaaacgtgggggcactgtccctgtagcctg |
| | aagaccagcaggagacgatcggaagcatcacagcacaaccggtatcgatagcgtctgcgtgttgggagctggagtcgtgggcttgacg |
| | acggcgctgcagctcttgcaggatgtgcctggcgtgcgcgttcacgtcgtggctgagaaatatggcgacgaaacgttgacggctggg |
| | gccggcgggctgtggatgccatacgcattgggtacgcggccattggatgggattggtaggcttatggagggataatagagtttttgtc |
| | ggatccaacgcatgttgatgcagtagcccggtgtgctgaaagtatggaaggatggtgcattggctattcacatgegctgcccacccct |
| | tttcgcaggaaatgtgccggcatcgttggtgcaccgatggggaaaatcgacgttcgaccactacatgaaaatgtatacctctgaagat |
| | gcagcaactgcgggtgcgaaacggataacggtttggtaggtgtatgtcgcagcatgtgctggattttgcgggcaactccccctgccac |
| | ggcccattgcaggtgtcatgttgactggaggatacgaactttcggccgtcaaattcccagaggaggaccccctctgggccgacattgt |
| | gcccactgctcttccgcttgctgtttcctgtgtgaaattgt |

The construct can be written as 5′DAO1b::CrTUB2:ScSUC2:PmPGH:CvNR:PmAMT03:PedLPAAT2:PmPGH::3′DAO1b. Proceeding in the 5′ to 3′ direction bold, lowercase sequences represent genomic DNA from CHK22 that permit targeted integration at the DAO1b locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text that enables amplification of the PedLPAAT2 gene, followed by a linker, indicated by lowercase, bold italics, in which contains a C. vulgaris nitrate reductase 3′-UTR. The P. moriformis PmAMT03 promoter, indicated by boxed italicized text, drive the expression of the PedLPAAT2. The initiator ATG and terminator TAG codons of the PedLPAAT2 are indicated by uppercase, bold italics, while the remainder of the gene is indicated by bold italics. The P. moriformis PmPGH 3′-UTR is indicated by lowercase underlined text followed by the CHK22 DAO1b genomic region indicated by bold, lowercase text.

Table 56 shows the fatty acid/TAG profiles of primary transformants of CHK22 transformed with pCHK972. The P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 56
Screen of primary transformants of CHK22 with pCHK972.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK972- | 0.05 | 1.56 | 23.91 | 3.02 | 58.99 | 10.16 | 0.51 | 0.33 | 0.09 | 1.49 |
| 1 | | | | | | | | | | |
| pCHK972- | 0.04 | 1.54 | 25.41 | 3.08 | 59.09 | 8.65 | 0.48 | 0.30 | 0.09 | 1.86 |
| 2 | | | | | | | | | | |
| pCHK972- | 0.04 | 1.58 | 25.14 | 2.92 | 58.78 | 9.28 | 0.49 | 0.27 | 0.09 | 2.00 |
| 3 | | | | | | | | | | |
| pCHK972- | 0.05 | 1.64 | 20.42 | 3.47 | 57.89 | 14.28 | 0.56 | 0.17 | 0.00 | 4.47 |
| 4 | | | | | | | | | | |
| pCHK972- | 0.06 | 1.73 | 21.15 | 3.09 | 57.89 | 13.56 | 0.61 | 0.31 | 0.10 | 1.55 |
| 5 | | | | | | | | | | |
| pCHK972- | 0.05 | 1.69 | 20.40 | 3.21 | 57.44 | 14.85 | 0.57 | 0.21 | 0.00 | 4.33 |
| 6 | | | | | | | | | | |
| pCHK972- | 0.06 | 1.68 | 20.59 | 3.45 | 57.50 | 14.43 | 0.57 | 0.22 | 0.12 | 4.16 |
| 7 | | | | | | | | | | |
| pCHK972- | 0.05 | 1.63 | 24.13 | 3.09 | 58.15 | 10.67 | 0.52 | 0.27 | 0.09 | 2.09 |
| 8 | | | | | | | | | | |
| CHK22 | 0.04 | 1.50 | 26.06 | 3.13 | 59.36 | 7.76 | 0.46 | 0.32 | 0.00 | 1.58 |
| Control | | | | | | | | | | |

As the transformants of CHK22 expressing CrLPAAT2 produced the higher OP:OO ratio, the percent identity (%) of all the amino acid sequences across the entire LPAAT protein was compared against that of CrLPAAT2 the results are shown in Table 57. Pairwise identities (%) between all the sequences vs CrLPAAT2. The resulted phenotype of expressing these LPAATs in CHK22 is indicated with the ml/z (577.5/603.5) OP:OO ratio; the OP:00 ratio shown in the rows of PmLPAAT1 or PmLPAAT2 represents the analytical results of the non-transgenic line CHK22. Amino acid sequences of the LPAATs described herein are listed in Table 58.

TABLE 57
OP:OO Ratio

| | | | | m/z (577.5/603.5) |
|---|---|---|---|---|
| Gene Name | Organisms | GenBank ID | Identity (%) | OP:OO |
| CrLPAAT2 | Chlamydomonas | XP_001699586.2 | 100 | 3.48 |
| | reinhardtii | | | |
| VcLPAAT2 V2 | Volvox carteri f. | XP_002947580.1 | 34.36 | 1.59 |
| | nagariensis_120491 | | | |
| AgLPAAT2 | Astrephomene | GFR47784.1 | 69.10 | 2.21 |
| | gubernaculifera | | | |
| EdLPAAT2 | Edaphochlamys | KAG2489453.1 | 59.94 | 3.78 |
| | debaryana | | | |
| CeLPAAT2 | Chlamydomonas | GAX78532.1 | 55.91 | 1.59 |
| | eustigma | | | |
| DsLPAAT2 | Dunaliella salina | KAF5838861.1 | 44.85 | 2.35 |
| PedLPAAT2 | Pedinophyceae sp | CAG9465508.1 | 44.61 | 4.47 |
| SceLPAAT2 | Scenedesmus sp. | KAF6259536.1 | 39.76 | 1.64 |
| | NREL 46B-D3 | | | |
| McLPAAT2 | Micractinium | PSC72189.1 | 39.76 | 1.55 |
| | conductrix | | | |
| ChsoLPAAT2 | Chlorella | PRW57325.1 | 40.73 | 1.53 |
| | sorokiniana | | | |
| ChvaLPAAT2 | Chlorella variabilis | XP_005852243.1 | 39.21 | 1.58 |
| RsLPAAT2 | Raphidocelis | GBF93205.1 | 40.06 | 1.58 |
| | subcapitata | | | |
| ChdeLPAAT2 | Chlorella desiccata | KAH7617329.1 | 40.30 | 1.71 |
| ApLPAAT2 | Auxenochlorella | XP_011401335.1 | 37.05 | 1.58 |
| | protothecoides | | | |
| ChprLPAAT2 | Chloropicon primus | QDZ21586.1 | 34.13 | 1.62 |
| AGPAT1 | Homo sapiens | NP_001358366.1 | 11.32 | 3.60 |
| VrLPAAT2 | Volvox reticuliferus | GIM16764.1 | 80.77 | 1.69 |
| ChvuLPAAT2 | Chlorella vulgaris | KAI3438340.1 | 39.21 | 1.62 |

TABLE 58
LPAAT Amino Acid Sequences

| SEQ ID | | |
|---|---|---|
| NO: | LPAAT | Amino Acid Sequence |
| 40 | VcLPAAT2 | MSVPSKALSLPSFLFSVFVFYWSLPLFAIIYRIRFANVGKR |
| | V2 | NDMLDWAKALVAFFRVTVLKQGEMSLYRGGRCLYLCN |
| | | HRSWADFFIDTYLTEGRAALMSRWMVFFAFPVFCTSVLIL |
| | | RGIVLFKRGHIADKETASLQPIEEAFNVWLDATLRKSTVP |
| | | GLLVYPEGHRSLKPRSLPLKRGMLHFAFSRHLAVQLIITR |
| | | GKEEVIKPTDFDSFDAFFQEVQTTWDACWQLSYHQPTEG |
| | | VPRLSLKDAHEYDYPPSMWWLQLGVTLLSIVLLAAVLYG |
| | | SWLVLRAGILSLGSSGTQQTHRYVSDVPDLDQLTALQEF |
| | | MGLPGDVDLQDYADVPERRMTLVLDLVVGRTEESAKGC |
| | | CQKKRAVFEISRYRDYAALCTEGSQVMHALDGVITDLSN |
| | | GTCDSTESVERISALPAEGVGETGVEGARRLLNYISFFGTP |
| | | LGEGAWQACRTVMDRRARAASILDEPFTLEEVARAVGGL |
| | | PNGKAHGLEVAPSECYRYARLPRVDEQGRQGTEVNCIVP |
| | | VLHVLLAHIRTTGDFPQQFTKTVVSPILKKGDPLVAGNY |
| | | WGIAVGGAFAKCYASVILNRLVRAGETLDLRHPCQAGFR |
| | | LGFGTAHHLFVKQHLVRRRRSGGCPALRCRRLPRR |
| 41 | AgLPAAT2 | MLCLYRSAVIFILSSSVLAFICHFLGALGACVLWGQLSLSS |
| | | RNLFGLRCNCCLLTDSMEASSKVLTFPAFLCSVFVFYWSL |
| | | PIFAIIYRIRFANLGKRNDMLEWAKSLVRFFGVTVLKEGES |
| | | SLYRGGKCLYLCNHRSWADFFIDAYLTEGRAALMSRWL |
| | | VYFVFPVFCTSCLILRGIVLFKRGTIADKEAFNSWLDATLG |
| | | RSHVPGLLVYPEGHRSLKPASLPLKRGMLHYAFSRKIPVQ |
| | | IIITRGKEEVLSEKRMAVRFGRTLVTQFSKVIEPGQFASFD |
| | | AFFSAVQSTWDECWRGAYGASTTSTPRHTLSHPPTEYDY |
| | | PARMRLQQAAITLFSMVVLAGVLAGSWRAGAAV |
| 42 | EdLPAAT2 | MAVSKLLGLPAFLFSVFAFYWSLPMLAITYRLRFLNLGKR |
| | (Edaphochlamys | NDMLDWGRGLVNFFAVTIYKVGDGSLYKGGSCLYLCNH |
| | debaryana) | RSWADFFIDIYITEGRGAMMSRWLVFFVFPIFCSAVVTLK |
| | | GIILFKRGTIADKEKFNAWLDATLASSPVPGMLVYPEGHR |
| | | SIKPHSLPLKRGMLHYAFSRKLPVQVIITRGKDDVLSEKT |
| | | MAVHWRRQLVTSYGKVIKPSECASFDAFMHEVQTEWDK |
| | | HWAEAYNAPKPPSKSLPRLVPAPHGYDYPLSMRLGMGSV |
| | | TTFSIFAFAGVLYGTYCTALWVLAFTGAAQKVIVTVLAL |
| | | WMGLALTRAVL |
| 43 | DsLPAAT2 | MGQPLLQKFISLPTFLLAIFLLYWSLPICVLVGTLKLLDFTF |
| | | PRTDMYNWCQFMTKLFRIKIKKYGPNGLRKDTGRHILYL |
| | | CNHRSWTDFFLDAYLTEGNGSLMSRWLVFAVFPVFMVA |
| | | VIVLRGVILFKRGTVLDKEKFNEWLDSKVSSSPIPGMLVY |
| | | PEGHRSLAHKSLPLKRGMLYYAHSRKFPVQIIISRNKEEVL |
| | | SEKTMKAHFGATIVTGFSELIESEGKDIKEFLSEVQTAWD |
| | | KMWDSVYSADAAALPELEHTDVPKFDYSFSMRVMQLLV |
| | | VSGTVVVFLGTMAAMVKCVCVCLAATGDLGCKVLVSSL |
| | | VGWFLVSILNSRAPSGK |
| 44 | SceLPAAT2 | MLLILKLPAFLLAVYVMYWSLPLANLLLFIKAWKVVGNR |
| | | NDMYRWCQFLVRACGIKVRKCGKQELYRGGACLYLAN |
| | | HRSWADFFVDAYLTEGRGQLMSRAAVIFVFPVVILPLTLV |
| | | RGCICFRRDGVKDKEKFNRWIDACIGASPVPGICLFPEGH |
| | | RSRLPHSLPLKRGMLNYAYSRKMPVQIVISAHKELAMDE |
| | | KSFHVRFGATCVTGYGPLMRTADYASFDEFYTALQGEW |
| | | QKLWVEVFSADPAALPELVISAPAGFTYPLQLKLSQLLLT |
| | | LLELALMAWGLAWSVRAWGGVAARLAGTLQLGVSGLQ |
| | | VLQYLPLVWLVVSLASCYDSTLPQPVLAADGTPLVGSSQ |
| | | ATRDSKKAL |
| 45 | McLPAAT2 | MRLGRAIARACRWPTFLGSILLLYWSVVCVALSYHFKWL |
| | | NLRGRRNDVLGWSGGMKWWVRAKVLKVGDRELYRGP |
| | | CVYLFNHRSWGDFIVDQYATEGRSLFMSRLTVLFVFPTF |
| | | MGALKVLKSVITFKRGAIADKERFNNWIDEQRAASPQTG |
| | | LSVYPEGHRSTAPVSLPLKRGMLYYAHSRRLPVQVIIGAN |
| | | KEAIISEKHCTARLNQTAAVGYSDPIMTADYPDFEAFWEQ |
| | | VQEAWNKTWAEVYGADWEGLPELAPGSAGWQDPDTHY |
| | | PLSVTLPMVGVVLANFAAFAGVSWLAFRALSALFALFGP |
| | | AQPLALALVAAYLALSFYLYSRPVDALQLHERLRRESKH |
| | | PSHLARSAAHLAGPRGVDANGAAAAEARVAALRHARPT |
| | | HRPTPYLGRRRPLAAPPARAPPRLSLRAPSAATAVARGML |
| 46 | ChsoLPAAT2 | MSVPQLVAKACRWPTFLGSIILLYWSVAFITLSGHLKWW |
| | | RLRGRRNDVLAWSAGMKWWVRCRVLKVGSTDLYRQPC |
| | | VYLFNHRSWGDFIVDQYVTEGRSLFMSRLAVLGVFPTFIY |
| | | SLVILKSIILFKRGSIADKQRFNAWIDKQREGSPQTGLAVY |
| | | PEGHRSTLGEPLPLKRGMLHYAYSRKLPVQIVIGANKEAII |
| | | AEKRCTARLGQTAVVGYSEPIITADYPDFEAFWAEVQEA |
| | | WDAQWQAVFGADWQGLPELAVQEPVTHYPLSITLPMLG |
| | | MTWLNLGLFAWTCWLTFRAVRWAFALFGPAQPLALAAA |
| | | AAYLALSFWLYSQPVNALKAMQTQRGHPGKLAESEANL |
| | | AALDGSGARRDS |
| 47 | ChvaLPAAT2 | MTLARLIAKATKWPTFLGSILLLYWSVVFVALSYHIKWL |
| | | KLKGRRNDVLGWAAGMKWWIRCRVLKVGTQELYRGQC |
| | | VYLFNHRSWGDFIVDQYVTEGRSLFMSRLAVLGVFPTFV |
| | | SALVVLKSIILFKRGSVADKEKFNRWIDEQRAASPQPGLS |
| | | VYPEGHRSTLGEPLPLKRGMLHYAFSRKLPIVIGANKEAII |
| | | SEKHCTARLNQTAVVGYSAPIMTANYPSFDAFWEEVQEA |
| | | WNAQWAAVFGADWDGLPELAPQEPDTEYPASITLGMLG |
| | | VVVANMALFCLTCWLSFRFWRWLFALFGPAQPVALAAA |
| | | AGYLALAFWLYSRPVNALQLHAEKRRPGQLAQSATNVA |
| | | ALDGKKAS |
| 48 | RsLPAAT2 | MLLLRLPAFWLAVWLMYWSLPLGVLTLYIPVWQLWGAR |
| | | NDTFYWVKFLVTALGIKFRKRGSGELYSGQAPCIYLANH |
| | | RSWADFFVDAYLAGGRAQMLSRMAVLYAFPMFMAPVIA |
| | | MRGCIVFKRGRIDDKEAFNKWIDGLVEASPQNGLMVYPE |
| | | GHRSTLAHSLPLKRGMLHYAYSRKMPVQIIMSANKESVL |
| | | SEKELHVGFGCTVVTSYSGLISPGDYAGFEGFMAAVQAA |
| | | WDKEWRDTMSADAAGLPPLHVSGTIDDNHYPPRLVVSQ |
| | | LFFSLLGITLLFAASSITWGWILRAKGVLGPQGAKMVVSF |
| | | LVAWFAASLAYCYNGQLPKPRLLPDGSEANGGGAGWKA |
| | | GGRPGGGAAAKGGALKAAAAGGGGGGSPKKSS |
| 49 | ChdeLPAAT2 | MSIPKALASALRWPPFLFSVFCLYWDVAILVLTYRIKWLD |
| | | FKKKRNDVLALGKGLKDIHRVTILKVGYRELYRKNCMYL |
| | | CNHRSWADFLVDQYVTEGRTLFVGRWAVAAAFPLVMIP |
| | | LRAIRCAILFKRGHIADIEGENKWIDSQFANSPQTGLGVYP |
| | | EGHRSTHGESLPLKRGMLKYAFSRKLPVQIVIGGNKESILS |
| | | EKHCTARFYQTIAVGFSEVLKPEEYDDFENFMQKVQETW |
| | | NREWSEVFSSNLKGLPVLPEVREPQLDYPMDVRMIMAIC |
| | | AVVNILMLAGVAWLTWWAVAAVMALMGPLQWPVAGL |
| | | VVAYIVASFYVYSKADDVRRTHQKMFQQRKQAPPIVSAA |
| | | GAGEGAGEGAGEENGDKKEQ |
| 50 | ApLPAAT2 | MSLQRTLDSALRWPAFLLSLFILFWSVPFCSLTYYIRFLDL |
| | | KTERRGDVFSWAVGMKRWFGIKILKVGDRELFRKDCMY |
| | | LINHRSWGDFLVDQYLTEGRSTFLARAMVAVAFPFFMTV |
| | | IMVMKATMVFKRGNIPDKEKFNRWIDSEFAATPQTAIAV |
| | | YPEGHRSTMTDSLPLKRGMLHYAYSRKLPVQVIIAANKE |
| | | AVLSEKHSTARFGQTIWAGYSEPMFPEKYETFDKFMDKV |
| | | QATWNEEWNTVFGTKPEGLRELVCEDPKLLTPLHVRRGV |
| | | STIIAVVDICFLAIMYYSLRMARRVSAALGPLEAPLLALLA |
| | | VYVTASLVRYSRPYDGVAASARKGKLAQASATQAPPAPK |
| | | LAASGVADVSAATINRAASLAAEPGDPQGRQ |
| 51 | ChprLPAAT2 | MKAIKDKMSFLFAMLIFYWSFPLTVAFGTDWSLSYKARN |
| | | DIFFWTAFLLGFFKVRTYRIGEQGCYRDGSCIWLCNHRD |
| | | WGDFFIDLYVVEGRACFLSRLMVLIAFPVFGFYGLVTRTA |
| | | VFFRRNTKGRDFTKFNQFISGQIQRSPCQGIVVYPEGTRNI |
| | | KPESLPLRRGMLRYAYSERTPVQIVITTNKEHVISQKMQR |
| | | ASFGTKCISGFSEVIRPGDYSNFEDFFDEINKTWKAEWKR |
| | | TYGASISNAVEVTPSEMKLYIPFYNIKLGQYLSMMAYSYF |
| | | TVALMVGVASFSFVLTQSMLGLRVVHFGGFSTDCIVVSIF |
| | | LGVFVACTKLITRKQD |
| 52 | AGPAT1 | MDLWPGAWMLLLLLFLLLLFLLPTLWFCSPSAKYFFKMA |
| | | FYNGWILFLAVLAIPVCAVRGRNVENMKILRLMLLHIKYL |
| | | YGIRVEVRGAHHFPPSQPYVVVSNHQSSLDLLGMMEVLP |
| | | GRCVPIAKRELLWAGSAGLACWLAGVIFIDRKRTGDAISV |
| | | MSEVAQTLLTQDVRVWVFPEGTRNHNGSMLPFKRGAFH |
| | | LAVQAQVPIVPIVMSSYQDFYCKKERRFTSGQCQVRVLPP |
| | | VPTEGLTPDDVPALADRVRHSMLTVFREISTDGRGGGDY |
| | | LKKPGGGG |
| 53 | CeLPAAT2 | MSIPTLLSIPYFAACVFVFYWSNTLGCLCYKIKFMNLIGPR |
| | | KDMHDWSEFMNWFFGVQYRQVGEQSLHKGERCLYLSN |
| | | HRSWADFFVDIYLTEGMAAPMSRNLVFYVFPFFMTSVSIL |
| | | KGIILFKRGVIEDKQKFNAWLDSKLDSSIRPSLLVYPEGHR |
| | | STRASSLPLKRGMLHYAHSRKLPIQIIMTKGKEGVLSEKH |
| | | MRASYGVTLATGFSEVIKSSNFPEFEAFALKVQQVWDAK |
| | | WQEVYTADISRRPDC |
| 54 | PedLPAAT2 | MNLAFPLAVAVMYWSLPIGALLYRIRFLNVFGPRNDMLE |
| | | WAEMMRAFLGVRFKLVGQRRLHKGERMVYLANHRSWA |
| | | DFFLDVWTTEARCSIMSRWMVFFAFPVFMASVIVLRGIVL |
| | | FKRGKIKDKDAFNAWLDKTLDRQVNSGSLLLYPEGTRNT |
| | | KPHALPLKRGMLKYSWSRGLPFQIIVTTNKERVLSEKKM |
| | | AVKFGVTCCTGFSEVIESKRYKDDFDGFMEEVQRVWDKE |
| | | WAEVYKAAAEPEKLEDLTIPSPDGFDYLPSMKLLQVLVT |
| | | LLTIAVFSAMVVGSAYALGLVVPAAQDAWKYASYVLAS |
| | | MPILGLASKLLGGSKVGAIKYPGLASKKID |
| 55 | VrLPAAT2 | MSVISKALSFPSFLFSVFVFYWSLPLFAIIYRIRFMNVSKRN |
| | | DMLDWAKALVAFFRVTVLKQGEMTLYRGARCLYLCNH |
| | | RSWADFFIDTYLTEGRAALMSRWLVYFVFPVFCTSVIILR |
| | | GIVLFKRGHIANKEAFNAWLDATLAKSTVPGLLVYPE |
| 56 | ChvuLPAAT2 | MPVPRIVAKATRWPTFLGSILLLFWSVVCVALSYHIKWLK |
| | | LRGKRNDVLGWAAGMKWWIRCRVLKVGSQDLYREKCV |
| | | YLFNHRSWGDFIVDQYVTEGRSLFMSRMAVLFVFPTFISA |
| | | LTVLKSIILFKRGAIADKEKFNAWIDEQRANSPQSGLSVYP |
| | | EGHRSTKRDSLPLKRGMLYYAFSRKLPVQIVIGANKEAIIS |
| | | EKHLTARLGQTAVVGYSEPIMTAEYADFDAFWAKVQET |
| | | WDRQWQTVFGAAWEGLPELEPVEPDTEYPLSITGRMVLV |
| | | VIANLLLFCCACWLTFHFWTWLFALFGPLQPAALALSAT |
| | | YLALALWIYSQPVNALQVHAKKLRPGQPAQPAVDMAAL |
| | | LETKKAS |

### Example 28: Expressing Volvax reticuliferus LPAAT2 (VrLPAAT2) in P. moriformis Strain CHK22

VrLPAAT2 was introduced into a P. moriformis strain CHK22. The expression construct pCHK1133 contained 5′ and 3′ homology arms to permit targeted integration of VrLPAAT2 into the genome and is shown in Table 59.

TABLE 59
Integrative sequences for the transformation of P. moriformis with pCHK1133
encoding VrLPAAT2.

| SEQ | |
|---|---|
| ID | |
| NO: | Nucleotide Sequence |
| 57 | gctcttccgcttgcccgcaccctegttgatctgggagccctgcgcagccccttaaatcatctcagtcaggtttctatgttcaactgagcct |
| | aaagggctttcgtcacgcgcacgagcacacgtatatcggctacgcagtctctcagaageggtagaacagttcgcaagccctcgtcgg |
| | tcgaaaacttgcgccagtactattgaattaaattaaatgatcgaatgagacgcgaaacttttgcagaatgccactgagtttgcccaga |
| | gaatgggagtggcgccattcaccatccgcctgtgcacggcctgattcgccgagacgatgaatggcgagaccagggagcggcttgcg |
| | agccccgagccggtagcaggaataacggtcgacaatcttcctgtccaattactggcaaccattagaaagagccggagcgcgttgaa |
| | attctgcaatcgagtaatttttcgatgcggcgggcctgctgaaccctaaggctccggcctatgtttaaggcgatccaagatgcacgcgg |
| | ccccaggcacgtgtctcaagcacaaaccccagccttagtttcgagactttgggagatagcggcagatatctagtttggcattttgtatat |
| | taattaagtctcgcaatggagcgctctgatgcggtgcagcgtcggctgcagcacctggcagtggcgctggggtcgccctatcgctcgg |
| | cgcctggtcagctggctcccgcctcctgcggcgcgcc 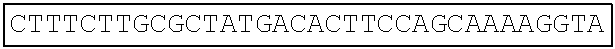 |
| | 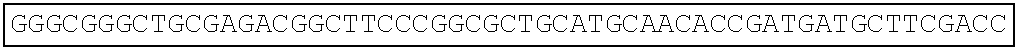 |
| | 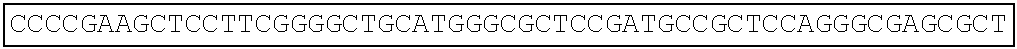 |
| | 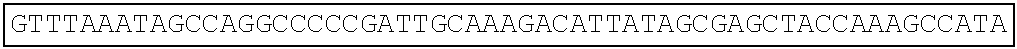 |
| | 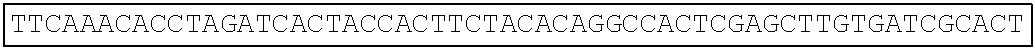 |
| | 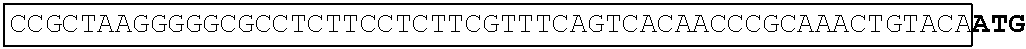 |
| | ctgctgcaggccttcctgttcctgctggccggcttcgccgccaagatcagcgcctccatgacgaacgagacgtccgaccgccccctggtg |
| | cacttcacccccaacaagggctggatgaacgaccccaacggcctgtggtacgacgagaaggacgccaagtggcacctgtacttccagt |
| | acaacccgaacgacaccgtctgggggacgcccttgttctggggccacgccacgtccgacgacctgaccaactgggaggaccagccca |
| | tcgccatcgccccgaagcgcaacgactccggcgccttctccggctccatggtggtggactacaacaacacctccggcttcttcaacgaca |
| | ccatcgacccgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctggtacgagc |
| | cctcccagaagtggatcatgaccgcggccaagtcccaggactacaagatcgagatctactcctccgacgacctgaagtcctggaagctg |
| | gagtccgcgttcgccaacgagggcttcctcggctaccagtacgagtgccccggcctgatcgaggtccccaccgagcaggaccccagca |
| | agtcctactgggtgatgttcatctccatcaaccccggcgccccggccggcggctccttcaaccagtacttcgtcggcagcttcaacggcac |
| | ccacttcgaggccttcgacaaccagtcccgcgtggtggacttcggcaaggactactacgccctgcagaccttcttcaacaccgacccgac |
| | ctacgggagcgccctgggcatcgcgtgggcctccaactgggagtactccgccttcgtgcccaccaacccctggcgctcctccatgtccctc |
| | gtgcgcaagttctccctcaacaccgagtaccaggccaacccggagacggagctgatcaacctgaaggccgagccgatcctgaacatc |
| | agcaacgccggcccctggagccggttcgccaccaacaccacgttgacgaaggccaacagctacaacgtcgacctgtccaacagcacc |
| | ggcaccctggagttcgagctggtgtacgccgtcaacaccacccagacgatctccaagtccgtgttcgcggacctctccctctggttcaagg |
| | gcctggaggaccccgaggagtacctccgcatgggcttcgaggtgtccgcgtcctccttcttcctggaccgcgggaacagcaaggtgaag |
| | ttcgtgaaggagaacccctacttcaccaaccgcatgagcgtgaacaaccagcccttcaagagcgagaacgacctgtcctactacaagg |
| | tgtacggcttgctggaccagaacatcctggagctgtacttcaacgacggcgacgtcgtgtccaccaacacctacttcatgaccaccggga |
| | acgccctgggctccgtgaacatgacgacgggggtggacaacctgttctacatcgacaagttccaggtgcgcgaggtcaagtgactTA |
| | AGACGCCCGCGCGGCGCACCTGACCTGTTCTCTCGAGGGCGCCTGTTCTGCCTTGCGA |
| | AACAAGCCCCTGGAGCATGCGTGCATGATCGTCTCTGGCGCCCCGCCGCGCGGTTTGT |
| | CGCCCTCGCGGGCGCCCCGGCCGCGGGGGCGCATTGAAATTGTTGCAAACCCCACCT |
| | GACAGATTGAGGGCCCAGGCAGGAAGGCGTTGAGATGGAGGTACAGGAGTCAAGTA |
| | ACTGAAAGTTTTTATGATAACTAACAACAAAGGGTCGTTTCTGGCCAGCGAATGACA |
| | AGAACAAGATTCCACATTTCCGTGTAGAGGCTTGCCATCGAATGTGAGCGGGCGGGC |
| | CGCGGACCCGACAAAACCCTTACGACGTGGTAAGAAAAACGTGGCGGGCACTGTCCC |
| | TGTAGCCTGAAGACCAGCAGGAGACGATCGGAAGCATCACAGCACAACCGGtgcagcag |
| | cagctcggatagtatcgacacactctggacgctggtcgtgtgatggactgttgccgccacacttgctgccttgacctgtgaatatccctgcc |
| | gcttttatcaaacagcctcagtgtgtttgatcttgtgtgtacgcgcttttgcgagttgctagctgcttgtgctatttgcgaataccacccccagca |
| | tccccttccctegtttcatatcgcttgcatcccaaccgcaacttatctacgctgtcctgctatccctcagegctgctcctgctcctgctcactgcc |
| | cctcgcacagccttggtttgggctccgcctgtattctcctggtactgcaacctgtaaaccagcactgcaatgctgatgcacgggaagtagtg |
| | ggatgggaacacaaatggacctaggaaataacaatca 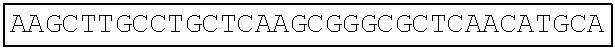 |
| | 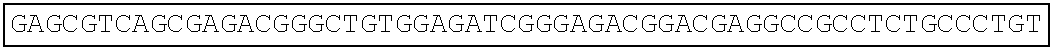 |
| | 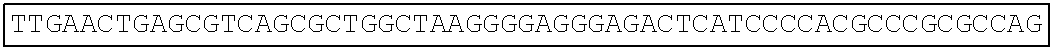 |
| | 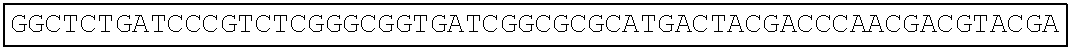 |
| | 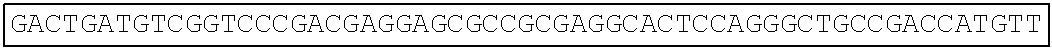 |
| | 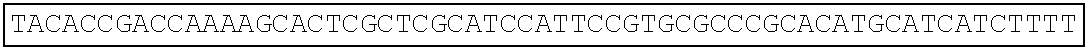 |
| | 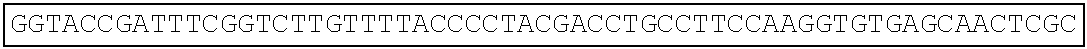 |
| | 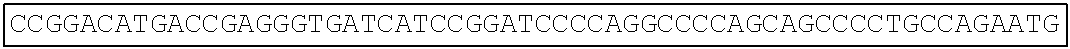 |
| | 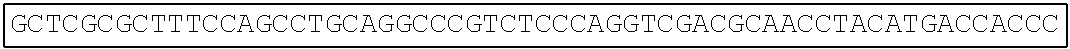 |
| | 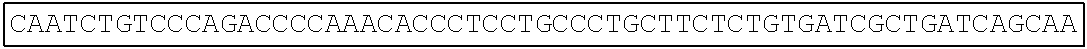 |
| | 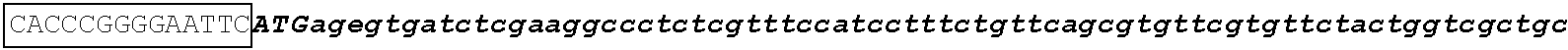 |
| | cgctgtttgcgatcatttaccgcatccgtttcatgaacgtgtcgaagcgcaacgacatgctcgactgggcgaagggctegtcgcctttttcc |
| | gcgtcaccgtgctgaagcagggcgagatgaccctctaccgcggcgcccgatgcctgtacctgtgcaaccaccggagctgggcggatttt |
| | ttcatcgacacctacctgacggagggccgggcggccctgatgtegcgctggctcgtctactttgtcttcccggtgttctgcacctccgtcatta |
| | tcctgcgtgggatcgtgctgttcaagcgtgggcatatcgccaacaaggaggccttcaacgcgtggctcgacgccacgctggccaagtcc |
| | acggtgccgggcctcctggtctaccccgagTAGacgcccgcgcggcgcacctgacctgttctctcgagggcgcctgttctgccttgcgaa |
| | acaagcccctggagcatgcgtgcatgatcgtctctggcgccccgccgcgcggtttgtcgccctcgcgggcgccgcggccgcgggggcgc |
| | attgaaattgttgcaaaccccacctgacagattgagggcccaggcaggaaggcgttgagatggaggtacaggagtcaagtaactgaaagtttt |
| | tatgataactaacaacaaagggtcgtttctggccagcgaatgacaagaacaagattccacatttccgtgtagaggcttgccatcgaatgtgagc |
| | gggcgggccgcggacccgacaaaacccttacgacgtggtaagaaaaacgtgggggcactgtccctgtagcctgaagaccagcaggaga |
| | cgatcggaagcatcacagcacaggccggcccgcgtctcgaacagagcgcggcggccgcatcgatacctgcaggaagcgtctgcgtgttg |
| | ggagctggagtcgtgggcttgacgacggcgctgcagctcttgcaggatgtgcctggcgtgcgcgttcacgtcgtggctgagaaatatg |
| | gcgacgaaacgttgacggctggggccggcgggctgtggatgccatacgcattgggtacgcggccattggatgggattggtaggctta |
| | tggagggataatagagtttttgtcggatccaacgcatgttgatgcagtagcccggtgtgctgaaagtatggaaggatggtgcattggc |
| | tattcacatgcgctgcccaccccttttcgcaggaaatgtgccggcatcgttggtgcaccgatggggaaaatcgacgttcgaccactac |
| | atgaaaatgtatacctctgaagatgcagcaactgcgggtgcgaaacggataacggtttggtaggtgtatgtcgcagcatgtgctggat |
| | tttgcgggcaactccccctgccacggcccattgcaggtgtcatgttgactggaggatacgaactttcggccgtcaaattcccagagga |
| | ggaccccctctgggccgacattgtgcccactgctcttccgcttgctgtttcctgtgtgaaattgt |

The construct can be written as 5′DAO1b::CrTUB2:ScSUC2:PmPGH:CvNR:PmACP:VrLPAAT2:PmPGH::3′DAO1b. Proceeding in the 5′ to 3′ direction bold, lowercase sequences represent genomic DNA from CHK22 that permit targeted integration at the DAO1b locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text that enables amplification of the VrLPAAT2 gene, followed by a linker, indicated by lowercase, bold italics, in which contains a C. vulgaris nitrate reductase 3′-UTR. An acyl carrier protein promoter from P. moriformis (PmACP), indicated by boxed italicized text, drive the expression of the VrLPAAT2. The initiator ATG and terminator TAG codons of the VrLPAAT2 are indicated by uppercase, bold italics, while the remainder of the gene is indicated by bold italics. The P. moriformis PmPGH 3′-UTR is indicated by lowercase underlined text followed by the CHK22 DAO1b genomic region indicated by bold, lowercase text.

Table 60 shows the fatty acid/TAG profiles of primary transformants of CHK22 transformed with pCHK1133. The P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 60
Screen of primary transformants of CHK22 with pCHK1133.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK1133-1 | 0.04 | 1.13 | 21.73 | 3.26 | 62.52 | 9.06 | 0.54 | 0.28 | 0.08 | 1.59 |
| pCHK1133-2 | 0.03 | 1.14 | 23.45 | 3.14 | 61.66 | 8.30 | 0.55 | 0.28 | 0.08 | 1.63 |
| pCHK1133-3 | 0.03 | 1.07 | 21.91 | 3.17 | 62.82 | 8.72 | 0.55 | 0.28 | 0.08 | 1.59 |
| pCHK1133-4 | 0.03 | 1.12 | 22.63 | 3.20 | 62.35 | 8.40 | 0.52 | 0.28 | 0.08 | 1.53 |
| pCHK1133-5 | 0.03 | 1.11 | 22.07 | 3.04 | 62.71 | 8.72 | 0.54 | 0.27 | 0.08 | 1.59 |
| pCHK1133-6 | 0.04 | 1.14 | 22.35 | 3.06 | 62.05 | 9.04 | 0.56 | 0.28 | 0.09 | 1.46 |
| pCHK1133-7 | 0.03 | 1.21 | 26.33 | 3.04 | 60.23 | 6.93 | 0.53 | 0.25 | 0.07 | 1.54 |
| pCHK1133-8 | 0.03 | 1.13 | 23.45 | 3.06 | 61.89 | 8.14 | 0.53 | 0.28 | 0.08 | 1.57 |
| pCHK1133-9 | 0.04 | 1.36 | 27.75 | 2.64 | 56.65 | 8.95 | 0.61 | 0.24 | 0.07 | 1.69 |
| pCHK1133- | 0.03 | 1.14 | 24.06 | 3.13 | 61.71 | 7.70 | 0.52 | 0.28 | 0.07 | 1.56 |
| 10 | | | | | | | | | | |
| CHK22 | 0.03 | 1.23 | 26.94 | 3.06 | 60.23 | 6.39 | 0.50 | 0.23 | 0.06 | 1.52 |
| Control | | | | | | | | | | |

### Example 29: Expressing Chlorella vulgaris LPAAT2 (ChvuLPAAT2) in P. moriformis Strain CHK22

ChvuLPAAT2 was introduced into a P. moriformis strain CHK22. The expression construct pCHK1134 contained 5′ and 3′ homology arms to permit targeted integration of ChvuLPAAT2 into the genome and is shown in Table 61.

TABLE 61
Integrative sequences for the transformation of P. moriformis with pCHK1134
encoding ChvuLPAAT2.

| SEQ | |
|---|---|
| ID | |
| NO: | Nucleotide Sequence |
| 58 | gctcttccgcttgcccgcaccctcgttgatctgggagccctgcgcagccccttaaatcatctcagtcaggtttctatgttcaactgagcct |
| | aaagggctttcgtcacgcgcacgagcacacgtatatcggctacgcagtctctcagaagcggtagaacagttcgcaagccctcgtcgg |
| | tcgaaaacttgcgccagtactattgaattaaattaaatgatcgaatgagacgcgaaacttttgcagaatgccactgagtttgcccaga |
| | gaatgggagtggcgccattcaccatccgcctgtgcacggcctgattcgccgagacgatgaatggcgagaccagggagcggcttgcg |
| | agccccgagccggtagcaggaataacggtcgacaatcttcctgtccaattactggcaaccattagaaagagccggagcgcgttgaa |
| | attctgcaatcgagtaatttttcgatgcggcgggcctgctgaaccctaaggctccggcctatgtttaaggcgatccaagatgcacgcgg |
| | ccccaggcacgtgtctcaagcacaaaccccagccttagtttcgagactttgggagatagcggcagatatctagtttggcattttgtatat |
| | taattaagtctcgcaatggagcgctctgatgcggtgcagcgtcggctgcagcacctggcagtggcgctggggtcgccctatcgctcgg |
| | cgcctggtcagctggctcccgcctcctgcggcgcgcc 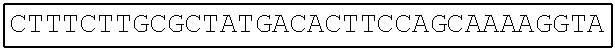 |
| | 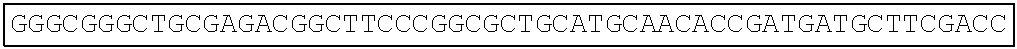 |
| | 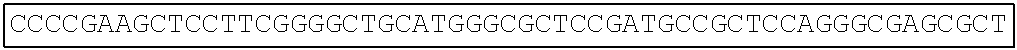 |
| | 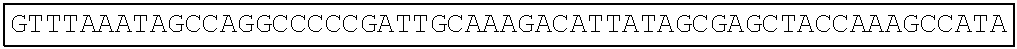 |
| | 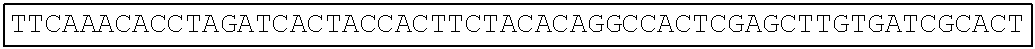 |
| | 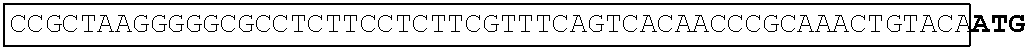 |
| | ctgctgcaggccttcctgttcctgctggccggcttcgccgccaagatcagcgcctccatgacgaacgagacgtccgaccgccccctggtg |
| | cacttcacccccaacaagggctggatgaacgaccccaacggcctgtggtacgacgagaaggacgccaagtggcacctgtacttccagt |
| | acaacccgaacgacaccgtctgggggacgcccttgttctggggccacgccacgtccgacgacctgaccaactgggaggaccagccca |
| | tcgccatcgccccgaagcgcaacgactccggcgccttctccggctccatggtggtggactacaacaacacctccggcttcttcaacgaca |
| | ccatcgacccgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctggtacgagc |
| | cctcccagaagtggatcatgaccgcggccaagtcccaggactacaagatcgagatctactcctccgacgacctgaagtcctggaagctg |
| | gagtccgcgttcgccaacgagggcttcctcggctaccagtacgagtgccccggcctgatcgaggtccccaccgagcaggaccccagca |
| | agtcctactgggtgatgttcatctccatcaaccccggcgccccggccggcggctccttcaaccagtacttcgtcggcagcttcaacggcac |
| | ccacttcgaggccttcgacaaccagtcccgcgtggtggacttcggcaaggactactacgccctgcagaccttcttcaacaccgacccgac |
| | ctacgggagcgccctgggcatcgcgtgggcctccaactgggagtactccgccttcgtgcccaccaacccctggcgctcctccatgtccctc |
| | gtgcgcaagttctccctcaacaccgagtaccaggccaacccggagacggagctgatcaacctgaaggccgagccgatcctgaacatc |
| | agcaacgccggcccctggagccggttcgccaccaacaccacgttgacgaaggccaacagctacaacgtcgacctgtccaacagcacc |
| | ggcaccctggagttcgagctggtgtacgccgtcaacaccacccagacgatctccaagtccgtgttcgcggacctctccctctggttcaagg |
| | gcctggaggaccccgaggagtacctccgcatgggcttcgaggtgtccgcgtcctccttcttcctggaccgcgggaacagcaaggtgaag |
| | ttcgtgaaggagaacccctacttcaccaaccgcatgagcgtgaacaaccagcccttcaagagcgagaacgacctgtcctactacaagg |
| | tgtacggcttgctggaccagaacatcctggagctgtacttcaacgacggcgacgtcgtgtccaccaacacctacttcatgaccaccggga |
| | acgccctgggctccgtgaacatgacgacgggggtggacaacctgttctacatcgacaagttccaggtgcgcgaggtcaagtgactTA |
| | AGACGCCCGCGCGGCGCACCTGACCTGTTCTCTCGAGGGCGCCTGTTCTGCCTTGCGA |
| | AACAAGCCCCTGGAGCATGCGTGCATGATCGTCTCTGGCGCCCCCCGCGCGGTTTGT |
| | CGCCCTCGCGGGCGCCCCGGCCGCGGGGGCGCATTGAAATTGTTGCAAACCCCACCT |
| | GACAGATTGAGGGCCCAGGCAGGAAGGCGTTGAGATGGAGGTACAGGAGTCAAGTA |
| | ACTGAAAGTTTTTATGATAACTAACAACAAAGGGTCGTTTCTGGCCAGCGAATGACA |
| | AGAACAAGATTCCACATTTCCGTGTAGAGGCTTGCCATCGAATGTGAGCGGGGGGC |
| | CGCGGACCCGACAAAACCCTTACGACGTGGTAAGAAAAACGTGGCGGGCACTGTCCC |
| | TGTAGCCTGAAGACCAGCAGGAGACGATCGGAAGCATCACAGCACAACCGGtgcagcag |
| | cagctcggatagtatcgacacactctggacgctggtcgtgtgatggactgttgccgccacacttgctgccttgacctgtgaatatccctgcc |
| | gcttttatcaaacagcctcagtgtgtttgatcttgtgtgtacgcgcttttgcgagttgctagctgcttgtgctatttgcgaataccacccccagca |
| | tccccttccctegtttcatategcttgcatcccaaccgcaacttatctacgctgtcctgctatccctcagegctgctcctgctcctgctcactgcc |
| | cctcgcacagccttggtttgggctccgcctgtattctcctggtactgcaacctgtaaaccagcactgcaatgctgatgcacgggaagtagtg |
| | ggatgggaacacaaatggacctaggaaataacaatca 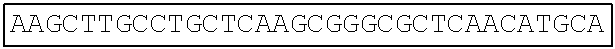 |
| | 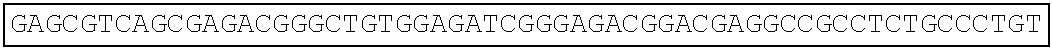 |
| | 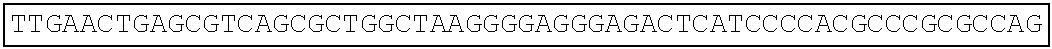 |
| | 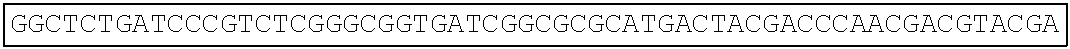 |
| | 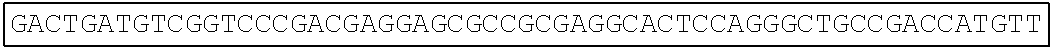 |
| | 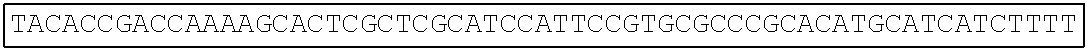 |
| | 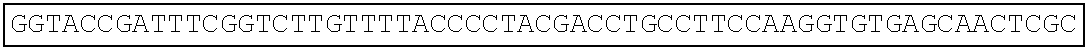 |
| | 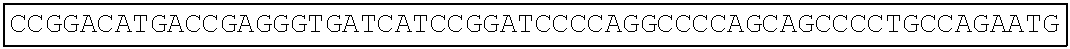 |
| | 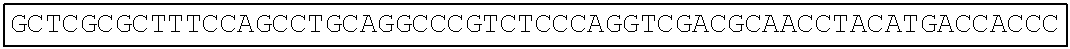 |
| | 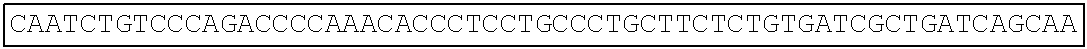 |
| | 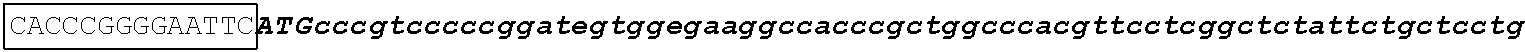 |
| | ttttggtctgtcgtgtgcgtcgcgctcagctaccacatcaagtggctgaagctccgcggcaagcgcaacgatgtgctgggctgggccgcg |
| | ggcatgaagtggtggattcgatgccgggtcctcaaggtgggctcgcaggatctgtaccgcgagaagtgcgtctacctctttaaccaccgct |
| | cctggggagacttcatcgtggaccagtacgtcacggagggccggagcctcttcatgagccgcatggccgtcctgttcgtctttcccaccttt |
| | atctccgcgctgacggtgctgaagtcgattatcctgttcaagcggggtgccatcgccgacaaggagaagtttaacgcttggatcgacgag |
| | cagcgagccaactcgcctcagtcgggcctgtctgtgtacccagagggccaccgctcgacgaagcgcgactcgctgcccctcaagcgag |
| | ggatgctgtactacgcgttcagccgaaagctgcccgtgcagatcgtgatcggcgccaacaaggaggctattatcagcgagaagcacctc |
| | acggcgcggctggggcagaccgccgtggtcggctacagegagcccattatgaccgcggagtacgccgacttcgacgccttctgggcga |
| | aggtccaggagacgtgggatcgccagtggcagacggtcttcggcgcggcctgggagggcctgcctgagctggagcctgtggagccag |
| | acacggagtaccccctctccatcacgggccgcatggtgctggtggtcattgcgaacctgctcctgttctgctgtgcttgctggctgacgtttca |
| | tttctggacgtggctgttcgccctgttcggcccgctccagcccgctgccctggccctgtctgcgacctacctggccctggctctctggatctact |
| | ctcaacccgtgaacgccctccaggtgcacgcgaagaagctgcgtccgggccaacctgcccagcccgccgtggacatggeggccctect |
| | ggagaccaagaaggcgtcgTAGacgcccgcgcggcgcacctgacctgttctctcgagggcgcctgttctgccttgegaaacaagcccc |
| | tggagcatgcgtgcatgatcgtctctggcgccccgccgcgcggtttgtcgccctcgcgggcgccgcggccgcgggggcgcattgaaattgt |
| | tgcaaaccccacctgacagattgagggcccaggcaggaaggcgttgagatggaggtacaggagtcaagtaactgaaagtttttatgataact |
| | aacaacaaagggtcgtttctggccagcgaatgacaagaacaagattccacatttccgtgtagaggcttgccatcgaatgtgagcggggggc |
| | cgcggacccgacaaaacccttacgacgtggtaagaaaaacgtggcgggcactgtccctgtagcctgaagaccagcaggagacgatcgga |
| | agcatcacagcacaggccggcccgcgtctcgaacagagcgcggcggccgcatcgatacctgcaggaagcgtctgcgtgttgggagctg |
| | gagtcgtgggcttgacgacggcgctgcagctcttgcaggatgtgcctggcgtgcgcgttcacgtcgtggctgagaaatatggcgacg |
| | aaacgttgacggctggggccggcgggctgtggatgccatacgcattgggtacgcggccattggatgggattggtaggcttatggagg |
| | gataatagagtttttgtcggatccaacgcatgttgatgcagtagcccggtgtgctgaaagtatggaaggatggtgcattggctattcac |
| | atgcgctgcccaccccttttcgcaggaaatgtgccggcatcgttggtgcaccgatggggaaaatcgacgttcgaccactacatgaaa |
| | atgtatacctctgaagatgcagcaactgcgggtgcgaaacggataacggtttggtaggtgtatgtcgcagcatgtgctggattttgcg |
| | ggcaactccccctgccacggcccattgcaggtgtcatgttgactggaggatacgaacttteggccgtcaaattcccagaggaggaccc |
| | cctctgggccgacattgtgcccactgctcttccgcttgctgtttcctgtgtgaaattgt |

The construct can be written as 5′DAO1b::CrTUB2:ScSUC2:PmPGH:CvNR:PmACP:ChvuLPAAT2:PmPGH::3′DAO1b. Proceeding in the 5′ to 3′ direction bold, lowercase sequences represent genomic DNA from CHK22 that permit targeted integration at the DAO1b locus via homologous recombination, the C. reinharditi β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text that enables amplification of the ChvuLPAAT2 gene, followed by a linker, indicated by lowercase, bold italics, in which contains a C. vulgaris nitrate reductase 3′-UTR. The P. moriformis PmACP promoter, indicated by boxed italicized text, drives the expression of the ChvuLPAAT2. The initiator ATG and terminator TAG codons of the ChvuLPAAT2 are indicated by uppercase, bold italics, while the remainder of the gene is indicated by bold italics. The P. moriformis PmPGH 3′-UTR is indicated by lowercase underlined text followed by the CHK22 DAO1b genomic region indicated by bold, lowercase text.

Table 62 shows the fatty acid/TAG profiles of primary transformants of CHK22 transformed with pCHK1134. The P. moriformis base strain CHK22 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 62
Screen of primary transformants of CHK22 with pCHK1134.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK1134-1 | 0.05 | 1.33 | 25.28 | 3.25 | 58.77 | 8.99 | 0.53 | 0.26 | 0.06 | 1.63 |
| pCHK1134-2 | 0.03 | 1.15 | 24.68 | 2.96 | 61.25 | 7.70 | 0.52 | 0.25 | 0.07 | 1.61 |
| pCHK1134-3 | 0.04 | 1.12 | 21.46 | 2.93 | 62.37 | 9.76 | 0.57 | 0.25 | 0.08 | 1.49 |
| pCHK1134-4 | 0.03 | 1.16 | 24.92 | 3.05 | 61.37 | 7.26 | 0.51 | 0.26 | 0.07 | 1.55 |
| pCHK1134-5 | 0.03 | 1.25 | 26.47 | 3.00 | 59.93 | 7.10 | 0.50 | 0.24 | 0.07 | 1.6 |
| pCHK1134-6 | 0.04 | 1.16 | 22,29 | 2.94 | 61.31 | 9.94 | 0.55 | 0.23 | 0.08 | 1.51 |
| pCHK1134-7 | 0.04 | 1.19 | 22-15 | 3.00 | 61.86 | 9.39 | 0.54 | 0.27 | 0.08 | 1.58 |
| pCHK1134-8 | 0.04 | 1.16 | 22.78 | 3.11 | 62.01 | 8.59 | 0.54 | 0.27 | 0.08 | 1.62 |
| pCHK1134-9 | 0.04 | 1.21 | 21.83 | 2.81 | 61.24 | 10.47 | 0.58 | 0.24 | 0.08 | 1.52 |
| pCHK1134- | 0.04 | 1.20 | 22.03 | 2.95 | 61.11 | 10.32 | 0.55 | 0.24 | 0.08 | 1.56 |
| 10 | | | | | | | | | | |
| CHK22 | 0.03 | 1.23 | 26.94 | 3.06 | 60.23 | 6.39 | 0.50 | 0.23 | 0.06 | 1.52 |
| Control | | | | | | | | | | |

### Example 30: Generation of a Classically Improved Microalgal Strain that Produces a Triglyceride Oil Enriched in Palmitate

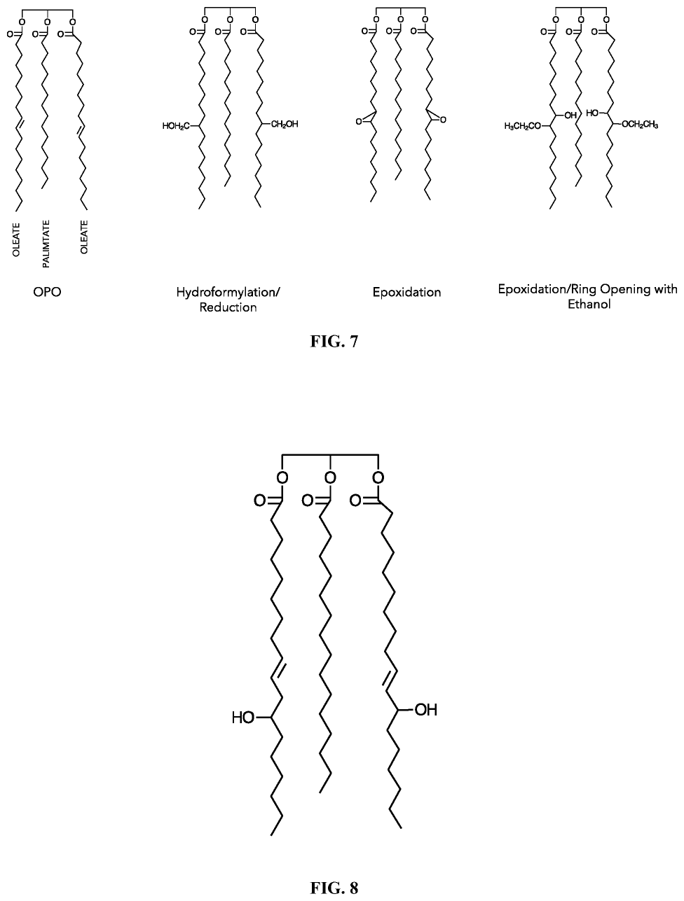

The microalgae P. moriformis strain CHK22 (UTEX 1533) was subjected to classical strain improvement to improve productivity, yield on carbon, and increase palmitic acid content. The mutagenesis, trait selection, and high-throughput, automated screening steps of the improvement process are outlined in the flow diagram in FIG. 9. Briefly, algal cells in log phase of growth were subjected to mutagenesis by treatment with chemicals or UV light (1). Cells were then sub-cultured into lipid production medium and subjected to selection/enrichment strategies (2). Strains were then plated to solid medium to obtain clonal isolates (3), followed by the interrogation of these isolates in lipid production medium in a 96-well format (4). Using glucose consumption as a surrogate for oil production, high glucose consuming strains were validated in lipid production medium in a tube or shake flask format (5). Successfully validated isolates were sub-cultured for multiple generations to stabilize mutations (6), followed by purification of clonal isolates (7), and subsequent re-interrogation in lipid production medium (8). Phenotypically stable clones were then validated in fermentation (9). Clones showing variability in (8) were revalidated and sub-cultured via (6) to generate stable lines.

CHK22 was chemically mutagenized for 30 min at 32° C. with 44 μM 4-nitroquinoline 1-oxide (4-NQO) or subjected to sham mutagenesis by the addition of only the mutagen solvent, DMSO. The mutagen was inactivated with the addition of sodium thiosulfate, which was then removed with repeated washing with water. The cells were then allowed to recover for 3 days in limited sugar growth media. The mutagenized and the mock-mutagenized populations were then independently cultured in lipid production media at 38° C. for five days. Optimal lipid production temperature for CHK22 is typically between 28-32° C. A higher-than-optimal temperature was applied as a stressor to provide a growth advantage to mutants having a predisposition toward producing higher palmitate levels. At the end of the 5-day, 38° C. lipid culture, the cells were collected and then incubated at 65° C. for 4 min. Based on range-finding experiments conducted in the same format, exposure to temperatures within this range for this duration of time typically kills >99% of the cell population. After heat exposure, the cells were recovered in limited sugar growth media for three days, diluted, and then plated. Mutant clones were then selected and assessed for glucose consumption rate and fatty acid profile in a 72h, 96-well block-based lipid production assay (as illustrated in FIG. 9). Table 63 summarizes glucose consumption rates and fatty acid profiles of TAG oil produced by the mutant strains. One mutant produced about 8% more palmitate than the CHK22 parent strain. This strain exhibited a significant reduction in glucose consumption rate, a hallmark of lower lipid titer.

TABLE 63
Fatty acid profiles of select mutant strains exhibiting higher glucose consumption
(Glc Rate) in a 96-well block lipid assay and increased palmitate (C16:0) levels.

| | Glc | | | | | | | | | | | | | | | 12- | |
|---|---|---|---|---|---|---|---|---|---|---|---|---|---|---|---|---|---|
| | Rate | C12: | C14: | C15: | C16: | C16: | C17: | C17: | C18: | C18: | C18: | C18:3 | C20: | C20: | C22: | OH, | C24: |
| Sample | (g/L*D) | 0 | 0 | 0 | 0 | 1 | 0 | 1 | 0 | 1 | 2 | alpha | 0 | 1 | 0 | 9_{ω}-C18:1 | 0 |
| CHK22 | 5.26 | 0.22 | 5.38 | 0.06 | 35.86 | 1.38 | 0.07 | 0.03 | 2.16 | 43.45 | 9.40 | 0.76 | 0.26 | 0.11 | 0.07 | 0.11 | 0.09 |
| mutant-1 | | | | | | | | | | | | | | | | | |
| CHK22 | 7.11 | 0.04 | 1.68 | 0.04 | 31.02 | 1.00 | 0.08 | 0.04 | 2.70 | 53.39 | 8.61 | 0.54 | 0.26 | 0.11 | 0.05 | 0.13 | 0.07 |
| mutant-2 | | | | | | | | | | | | | | | | | |
| CHK22 | 6.90 | 0.04 | 1.53 | 0.04 | 30.60 | 0.92 | 0.09 | 0.05 | 3.00 | 54.78 | 7.57 | 0.56 | 0.26 | 0.10 | 0.05 | 0.13 | 0.06 |
| mutant-3 | | | | | | | | | | | | | | | | | |
| CHK22 | 7.00 | 0.04 | 1.51 | 0.05 | 27.92 | 0.84 | 0.10 | 0.04 | 3.16 | 56.73 | 8.19 | 0.59 | 0.33 | 0.10 | 0.07 | 0.07 | 0.08 |
| Control | | | | | | | | | | | | | | | | | |
| CHK22 | 7.17 | 0.04 | 1.58 | 0.04 | 28.09 | 0.76 | 0.11 | 0.05 | 3.38 | 56.11 | 8.23 | 0.61 | 0.35 | 0.13 | 0.06 | 0.15 | 0.08 |
| Control | | | | | | | | | | | | | | | | | |
| CHK22 | 7.18 | 0.04 | 1.59 | 0.04 | 28.61 | 0.85 | 0.10 | 0.05 | 3.29 | 55.89 | 8.02 | 0.63 | 0.31 | 0.10 | 0.08 | 0.13 | 0.07 |
| Control | | | | | | | | | | | | | | | | | |

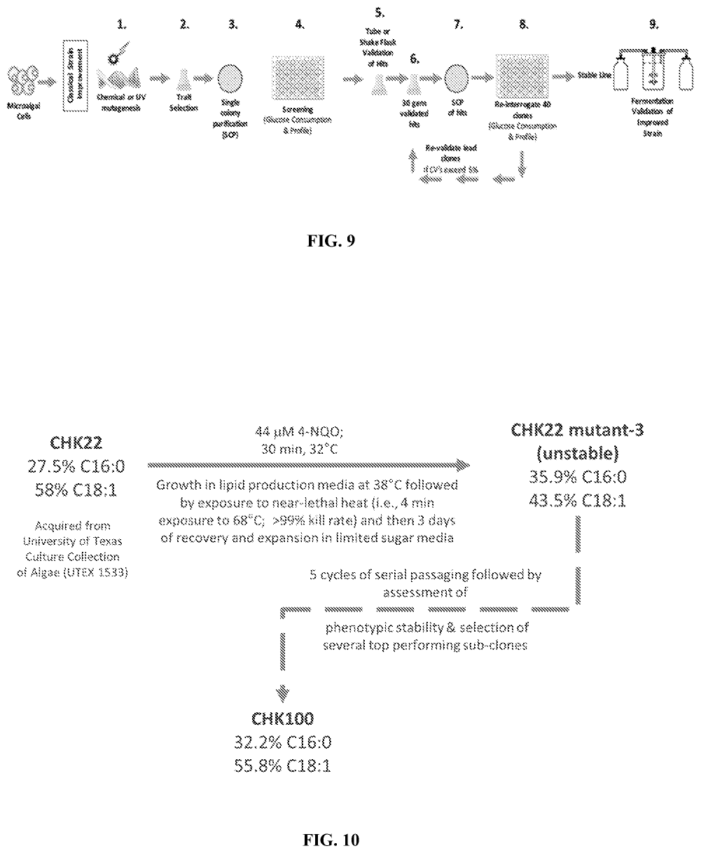

To further adapt the CHK22 mutant-3 strain to the altered fatty acid composition and improve lipid productivity, the strain was serially passaged in the absence of any selection for more than thirty population doublings and then plated to solid media to yield single colonies. These sub-clones were screened in a lipid production assay to assess the phenotypic stability of the lineage for both glucose consumption rate and fatty acid profile. After one cycle of serial passaging, the mutant was still phenotypically unstable. Several sub-clones that retained the higher palmitate level phenotype, along with increased rates of glucose consumption, were taken through additional rounds of serial passaging and stability assessment. After a total of five rounds of serial passaging and stability assessment, a stable strain, designated CHK100, was identified. CHK100 produced about 5% more palmitate than the CHK22 parental strain. The oil content (% on a weight-by-weight basis; % w/w), dry cell weight (DCW), and lipid titer (g/L) of CHK100 were also greater than those of CHK22 (Table 6 and Table 65). FIG. 10 illustrates the strain improvement strategy of CHK22 to produce CHK100.

TABLE 64
Tube-based assays on CHK100 showing improved lipid titer.

| | Oil | | | Avg. | Lipid | Avg. | | |
|---|---|---|---|---|---|---|---|---|
| | Content | Avg. | DCW | DCW | Titer | Lipid | NLB | |
| Sample | % w/w | % w/w | (g/L) | (g/L) | (g/L) | Titer (g/L) | (g/L) | PCP |
| CHK100-1 | 78.6 | 77.3 | 11.4 | 11.5 | 9.0 | 8.9 | 2.4 | 3.7 |
| CHK100-2 | 78.0 | | 11.3 | | 8.8 | | 2.5 | 3.5 |
| CHK100-3 | 75.5 | | 11.6 | | 8.8 | | 2.8 | 3.1 |
| CHK100-4 | 77.1 | | 11.6 | | 8.9 | | 2.7 | 3.4 |
| CHK22 | 77.7 | 76.2 | 8.8 | 9.2 | 6.8 | 7.0 | 2.0 | 3.5 |
| Control | | | | | | | | |
| CHK22 | 76.1 | | 9.1 | | 6.9 | | 2.2 | 3.2 |
| Control | | | | | | | | |
| CHK22 | 76.0 | | 9.4 | | 7.1 | | 2.3 | 3.2 |
| Control | | | | | | | | |
| CHK22 | 74.7 | | 9.6 | | 7.2 | | 2.4 | 3.0 |
| Control | | | | | | | | |

NLB, non-lipid biomass; PCP, per cell production.

TABLE 65
Tube-based assays on CHK100 showing increased C16:0 content.

| | C12: | C14: | C14: | C15: | C16: | C16: | C17: | C17: | C18: | C18: | C18: | C18:3 | C20: | C20: | C21: | C22: | |
|---|---|---|---|---|---|---|---|---|---|---|---|---|---|---|---|---|---|
| Sample | 0 | 0 | 1 | 0 | 0 | 1 | 0 | 1 | 0 | 1 | 2 | alpha | 0 | 1 | 0 | 0 | C24:0 |
| CHK100-1 | 0.03 | 1.22 | 0.02 | 0.03 | 32.25 | 1.36 | 0.06 | 0.04 | 2.03 | 55.65 | 6.36 | 0.50 | 0.19 | 0.05 | 0.05 | 0.04 | 0.00 |
| CHK100-2 | 0.03 | 1.22 | 0.03 | 0.03 | 32.25 | 1.36 | 0.07 | 0.04 | 2.02 | 55.61 | 6.37 | 0.53 | 0.19 | 0.05 | 0.05 | 0.04 | 0.00 |
| CHK100-3 | 0.03 | 1.18 | 0.02 | 0.03 | 32.07 | 1.40 | 0.07 | 0.03 | 1.99 | 55.89 | 6.27 | 0.57 | 0.18 | 0.05 | 0.05 | 0.03 | 0.00 |
| CHK100-4 | 0.03 | 1.18 | 0.02 | 0.04 | 32.15 | 1.38 | 0.07 | 0.03 | 1.99 | 55.91 | 6.25 | 0.55 | 0.19 | 0.05 | 0.04 | 0.03 | 0.00 |
| CHK22 | 0.04 | 1.39 | 0.02 | 0.03 | 27.59 | 0.85 | 0.09 | 0.03 | 3.27 | 57.92 | 7.45 | 0.64 | 0.33 | 0.07 | 0.06 | 0.06 | 0.07 |
| Control | | | | | | | | | | | | | | | | | |
| CHK22 | 0.04 | 1.39 | 0.02 | 0.03 | 27.62 | 0.85 | 0.10 | 0.04 | 3.27 | 57.88 | 7.44 | 0.63 | 0.32 | 0.07 | 0.06 | 0.06 | 0.06 |
| Control | | | | | | | | | | | | | | | | | |
| CHK22 | 0.04 | 1.36 | 0.02 | 0.04 | 27.39 | 0.85 | 0.10 | 0.03 | 3.27 | 58.28 | 7.30 | 0.68 | 0.31 | 0.07 | 0.06 | 0.06 | 0.06 |
| Control | | | | | | | | | | | | | | | | | |
| CHK22 | 0.04 | 1.36 | 0.02 | 0.03 | 27.37 | 0.85 | 0.9 | 0.04 | 3.29 | 58.34 | 7.30 | 0.67 | 0.32 | 0.07 | 0.05 | 0.06 | 0.06 |
| Control | | | | | | | | | | | | | | | | | |

All strains were subjected to standard lipid production conditions in which strains were grown in duplicate in 10 mL of lipid production medium. Cultures were grown with shaking (200 rpm) for 121 hours at 28° C. at which point about 1 mL of biomass was removed, applied to a polycarbonate filter, washed with an equal volume of Milli Q water, and placed in a tared glass vial at −80° C. for 30 minutes. Vials containing filters and frozen biomass were lyophilized to dryness overnight, weights were recorded, and filters with dried biomass were subjected to direct transesterification followed by GC/FID to quantitate FAMEs.

### Example 31: Expressing Pedinophyceae sp. LPAAT2 (PedLPAAT2) in P. moriformis Strain CHK100 Using the PmACP Promoter

PedLPAAT2 was introduced into a P. moriformis strain CHK22. The expression construct pCHK1126 contained 5Y and 3Y homology arms to permit targeted integration of PedLPAAT2 into the genome and is shown in Table 66.

TABLE 66
Integrative sequences for the transformation of P. moriformis with pCHK1126
encoding PedLPAAT2.

| SEQ | |
|---|---|
| ID | |
| NO: | Nucleotide Sequence |
| 59 | ccctcaactgcgacgctgggaaccttctccgggcaggcgatgtgcgtgggtttgcctccttggcacggctctacaccgtcgagtacgcc |
| | atgaggcggtgatggctgtgtcggttgccacttcgtccagagacggcaagtcgtccatcctctgcgtgtgtggcgcgacgctgcagca |
| | gtccctctgcagcagatgagcgtgactttggccatttcacgcactcgagtgtacacaatccatttttcttaaagcaaatgactgctgatt |
| | gaccagatactgtaacgctgatttcgctccagatcgcacagatagcgaccatgttgctgcgtctgaaaatctggattccgaattcgacc |
| | ctggcgctccatccatgcaacagatggcgacacttgttacaattcctgtcacccatcggcatggagcaggtccacttagattcccgatc |
| | acccacgcacatctcgctaatagtcattcgttcgtgtcttcgatcaatctcaagtgagtgtgcatggatcttggttgacgatgcggtatg |
| | ggtttgcgccgctggctgcagggtctgcccaaggcaagctaacccagctcctctccccgacaatactctcgcaggcaaagccggtca |
| | cttgccttccagattgccaataaactcaattatggcctctgtcatgccatccatgggtctgatgaatggtcacgctcgtgtcctgaccgtt |
| | 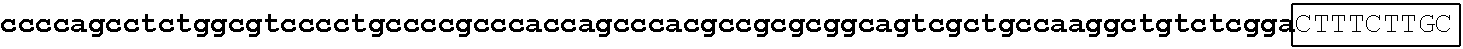 |
| | 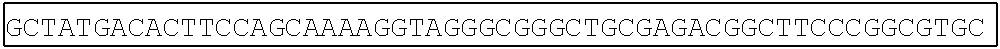 |
| | 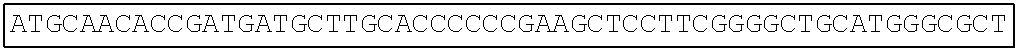 |
| | 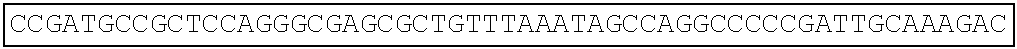 |
| | 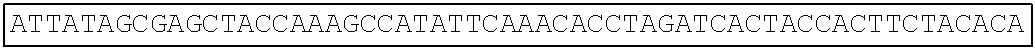 |
| | 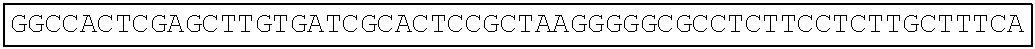 |
| | 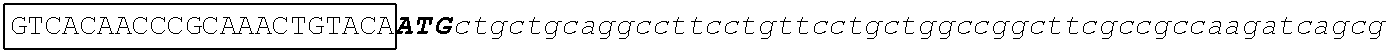 |
| | cctccatgacgaacgagacgtccgaccgccccctggtgcacttcacccccaacaagggctggatgaacgaccccaacggcctgtggta |
| | cgacgagaaggacgccaagtggcacctgtacttccagtacaacccgaacgacaccgtctgggggacgcccttgttctggggccacgcc |
| | acgtccgacgacctgaccaactgggaggaccagcccatcgccatcgccccgaagcgcaacgactccggcgccttctccggctccatgg |
| | tggtggactacaacaacacctccggcttcttcaacgacaccatcgacccgcgccagcgctgcgtggccatctggacctacaacaccccg |
| | gagtccgaggagcagtacatctcctacagcctggacggcggctacaccttcaccgagtaccagaagaaccccgtgctggccgccaact |
| | ccacccagttccgcgacccgaaggtcttctggtacgagccctcccagaagtggatcatgaccgcggccaagtcccaggactacaagat |
| | cgagatctactcctccgacgacctgaagtcctggaagctggagtccgcgttcgccaacgagggcttcctcggctaccagtacgagtgccc |
| | cggcctgatcgaggtccccaccgagcaggaccccagcaagtcctactgggtgatgttcatctccatcaaccccggcgccccggccggc |
| | ggctccttcaaccagtacttcgtcggcagcttcaacggcacccacttcgaggccttcgacaaccagtcccgcgtggtggacttcggcaag |
| | gactactacgccctgcagaccttcttcaacaccgacccgacctacgggagcgccctgggcatcgcgtgggcctccaactgggagtactc |
| | cgccttcgtgcccaccaacccctggcgctcctccatgtccctcgtgcgcaagttctccctcaacaccgagtaccaggccaacccggagac |
| | ggagctgatcaacctgaaggccgagccgatcctgaacatcagcaacgccggcccctggagccggttcgccaccaacaccacgttgac |
| | gaaggccaacagctacaacgtcgacctgtccaacagcaccggcaccctggagttcgagctggtgtacgccgtcaacaccacccagac |
| | gatctccaagtccgtgttcgcggacctctccctctggttcaagggcctggaggaccccgaggagtacctccgcatgggcttcgaggtgtcc |
| | gcgtcctccttcttcctggaccgcgggaacagcaaggtgaagttcgtgaaggagaacccctacttcaccaaccgcatgagcgtgaacaa |
| | ccagcccttcaagagcgagaacgacctgtcctactacaaggtgtacggcttgctggaccagaacatcctggagctgtacttcaacgacg |
| | gcgacgtcgtgtccaccaacacctacttcatgaccaccgggaacgccctgggctccgtgaacatgacgacgggggtggacaacctgttc |
| | tacatcgacaagttccaggtgcgcgaggtcaagtgactTAAGACGCCCGCGCGGCGCACCTGACCTGTTCT |
| | CTCGAGGGCGCCTGTTCTGCCTTGCGAAACAAGCCCCTGGAGCATGCGTGCATGATCG |
| | TCTCTGGCGCCCCGCCGCGCGGTTTGTCGCCCTCGCGGGCGCCGCGGCCGCGGGGGC |
| | GCATTGAAATTGTTGCAAACCCCACCTGACAGATTGAGGGCCCAGGCAGGAAGGCGT |
| | TGAGATGGAGGTACAGGAGTCAAGTAACTGAAAGTTTTTATGATAACTAACAACAAA |
| | GGGTCGTTTCTGGCCAGCGAATGACAAGAACAAGATTCCACATTTCCGTGTAGAGGC |
| | TTGCCATCGAATGTGAGCGGGCGGGCCGCGGACCCGACAAAACCCTTACGACGTGGT |
| | AAGAAAAACGTGGCGGGCACTGTCCCTGTAGCCTGAAGACCAGCAGGAGACGATCG |
| | GAAGCATCACAGCACAACCGGtgcagcagcagctcggatagtatcgacacactctggacgctggtcgtgtgatggact |
| | gttgccgccacacttgctgccttgacctgtgaatatccctgccgcttttatcaaacagcctcagtgtgtttgatcttgtgtgtacgcgcttttgcg |
| | agttgctagctgcttgtgctatttgcgaataccacccccagcatccccttccctcgtttcatatcgcttgcatcccaaccgcaacttatctacgc |
| | tgtcctgctatccctcagcgctgctcctgctcctgctcactgcccctcgcacagccttggtttgggctccgcctgtattctcctggtactgcaacc |
| | 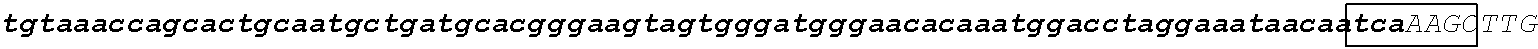 |
| | 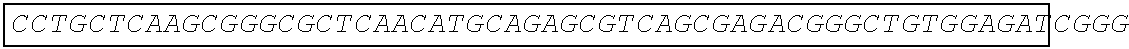 |
| | 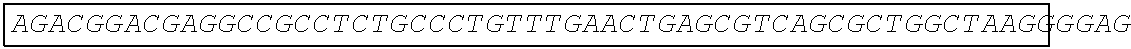 |
| | 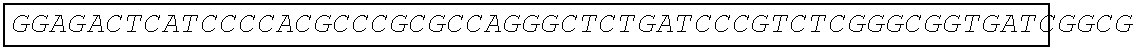 |
| | 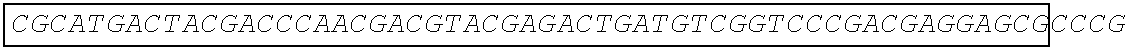 |
| | 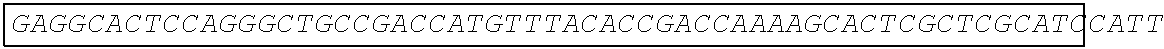 |
| | 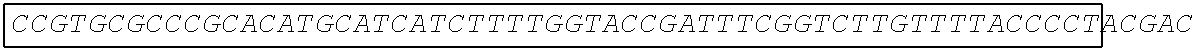 |
| | 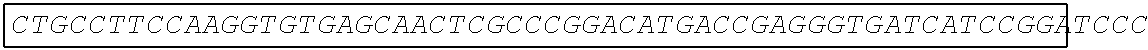 |
| | 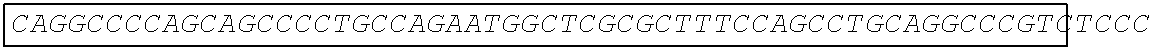 |
| | 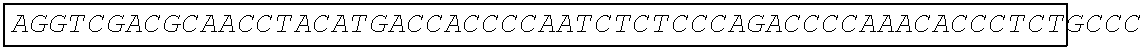 |
| | 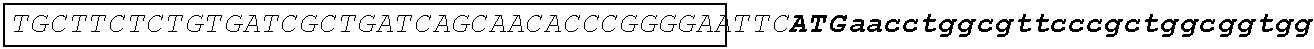 |
| | ccgtgatgtactggtccctcccgatcggagccctcctgtaccgcatccgctttctcaacgtgttcggcccccgtaacgatatgctggagtgg |
| | gcggagatgatgcgtgccttcctcggagtgcgcttcaagctcgtgggccaacgtcgcctgcacaagggcgagcgtatggtgtacctcgcc |
| | aaccaccgctcgtgggccgactttttcctggacgtgtggacgaccgaggcgcgctgctcgatcatgtcccgctggatggtcttctttgcctttc |
| | cggtgtttatggccagcgtcatcgtcctccgcggcatcgtgctgttcaagcggggcaagatcaaggacaaggacgcgtttaacgcctggc |
| | tggacaagaccctggaccgccaggtcaactccggctctctgctcctgtacccggaggggacgcgtaacacgaagcctcatgcgctgcct |
| | ctgaagcgcgggatgctgaagtactcctggagccgcggcctgccattccaaattatcgtcacgaccaacaaggagcgcgtcctgtcgga |
| | gaagaagatggcggtcaagttcggggtgacgtgctgtacggggttttccgaggtcatcgagtccaagcgctacaaggatgacttcgacg |
| | gctttatggaggaggtccagcgcgtgtgggataaggagtgggcggaggtgtacaaggccgctgcggagcccgagaagctggaggac |
| | ctcacgatccccagcccggacgggttcgactacctcccctcgatgaagctgctccaggtgctggtgaccctcctgaccatcgcggtgttca |
| | gcgcgatggtcgtgggctccgcctacgccctgggtctggtggtccccgcggcccaggacgcctggaagtacgcgagctacgtcctggcg |
| | tcgatgcccatcctgggcctcgccagcaagctgctcgggggcagcaaggtcggcgctatcaagtatccgggcctcgccagcaagaaga |
| | ttgacTGAacgcccgcgcggcgcacctgacctgttctctcgagggcgcctgttctgccttgcgaaacaagcccctggagcatgcgtgcatg |
| | atcgtctctggcgccccgccgcgcggtttgtcgccctcgcgggcgccccggccgcgggggcgcattgaaattgttgcaaaccccacctgac |
| | agattgagggcccaggcaggaaggcgttgagatggaggtacaggagtcaagtaactgaaagtttttatgataactaacaacaaagggtcgttt |
| | ctggccagcgaatgacaagaacaagattccacatttccgtgtagaggcttgccatcgaatgtgagcg gcgggccgcggacccgacaaaac |
| | ccttacgacgtggtaagaaaaacgtgggggcactgtccctgtagcctgaagaccagcaggagacgatcggaagcatcacagcacaggcc |
| | ggcccgcgtctcgaacagagcgcggcggccgcatcgatacctgcaggacagcgccatgccacgccctttgatggcttcaagtacgatta |
| | cggtgttggattgtgtgtttgttgcgtagtgtgcatggtttagaataatacacttgatttcttgctcacggcaatctcggcttgtccgcagg |
| | ttcaaccccatttcggagtctcaggtcagccgcgcaatgaccagccgctacttcaaggacttgcacgacaacgccgaggtgagctatg |
| | tttaggacttgattggaaattgtcgtcgacgcatattcgcgctccgcgacagcacccaagcaaaatgtcaagtgcgttccgatttgcgt |
| | ccgcaggtcgatgttgtgatcgtcggcgccggatccgccggtctgtcctgcgcttacgagctgaccaagcaccctgacgtccgggtac |
| | gcgagctgagattcgattagacataaattgaagattaaacccgtagaaaaatttgatggtcgcgaaactgtgctcgattgcaagaaa |
| | ttgatcgtcctccactccgcaggtcgccatcatcgagcagggcgttgctcccggcggcggcgcctggctggggggacagctgttctcg |
| | gccatgtgtgtacgtagaaggatgaatttcagctggttttcgttgcacagctgtttgtgcatgatttgtttcagactattgttgaatgttttt |
| | agatttcttaggatgcatgatttgtctgcatgcgact |

The construct can be written as 5′Thi4::CrTUB2:ScSUC2:PmPGH:CvNR:PmACP:PedLPAAT2:PmPGH::3′Thi4. Proceeding in the 5′ to 3′ direction bold, lowercase sequences represent genomic DNA from CHK100 that permit targeted integration at the Thi4 locus via homologous recombination, the C. reinhardtii β-tubulin promoter driving expression of the yeast sucrose invertase gene (conferring the ability of CHK100 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text that enables amplification of the PedLPAAT2 gene, followed by a linker, indicated by lowercase, bold italics, in which contains a C. vulgaris nitrate reductase 3′-UTR. The P. moriformis PmACP promoter, indicated by boxed italicized text, drives the expression of the PedLPAAT2. The initiator ATG and terminator TAG codons of the PedLPAAT2 are indicated by uppercase, bold italics, while the remainder of the gene is indicated by bold italics. The P. moriformis PmPGH 3′-UTR is indicated by lowercase underlined text followed by the CHK100 Thi4 genomic region indicated by bold, lowercase text.

Table 67 shows the fatty acid/TAG profiles of primary transformants of CHK100 transformed with pCHK1126. The P. moriformis base strain CHK100 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 67
Screen of primary transformants of CHK100 with pCHK1126.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK1126-1 | 0.03 | 1.24 | 29.75 | 2.10 | 57.26 | 7.38 | 0.39 | 0.17 | 0.07 | 3.79 |
| pCHK1126-2 | 0.03 | 1.23 | 30.71 | 2.19 | 55.74 | 7.74 | 0.49 | 0.19 | 0.07 | 4.42 |
| pCHK1126-3 | 0.03 | 1.28 | 32.21 | 2.28 | 54.06 | 7.59 | 0.69 | 0.21 | 0.07 | 4.5 |
| pCHK1126-4 | 0.03 | 1.24 | 30.53 | 2.22 | 56.36 | 7.40 | 0.42 | 0.17 | 0.07 | 4.31 |
| pCHK1126-5 | 0.03 | 1.33 | 32.07 | 2.29 | 55.48 | 6.47 | 0.42 | 0.19 | 0.05 | 1.47 |
| pCHK1126-6 | 0.04 | 1.69 | 31.44 | 2.23 | 55.42 | 6.84 | 0.47 | 0.18 | 0.06 | 4.46 |
| pCHK1126-7 | 0.03 | 1.30 | 32.18 | 2.28 | 55.38 | 6.49 | 0.45 | 0.18 | 0.05 | 1.52 |
| pCHK1126-8 | 0.03 | 1.24 | 30.45 | 2.32 | 56.35 | 7.40 | 0.45 | 0.18 | 0.07 | 4.72 |
| pCHK1126-9 | 0.03 | 1.25 | 30.99 | 2.30 | 55.94 | 7.29 | 0.43 | 0.18 | 0.07 | 4.39 |
| pCHK1126- | 0.03 | 1.18 | 29.08 | 1.41 | 57.06 | 8.73 | 0.28 | 0.12 | 0.08 | 4.35 |
| 10 | | | | | | | | | | |
| CHK100 | 0.03 | 1.33 | 32.22 | 2.29 | 55.28 | 6.50 | 0.44 | 0.18 | 0.05 | 1.60 |
| Control | | | | | | | | | | |

### Example 32: Expressing Pedinophyceae sp. LPAAT2 (PedLPAAT2) in P. moriformis Strain CHK100 Using the PmG3PDH Promoter

PedLPAAT2 was introduced into a P. moriformis strain CHK22. The expression construct pCHK1127 contained 5Y and 3Y homology arms to permit targeted integration of PedLPAAT2 into the genome and is shown in Table 68.

TABLE 68
Integrative sequences for the transformation of P. moriformis with pCHK1127
encoding PedLPAAT2.

| SEQ ID | |
|---|---|
| NO: | Nucleotide Sequence |
| 60 | ccctcaactgcgacgctgggaaccttctccgggcaggcgatgtgcgtgggtttgcctccttggcacggctctacaccgtcgagtacgcc |
| | atgaggcggtgatggctgtgtcggttgccacttcgtccagagacggcaagtcgtccatcctctgcgtgtgtggcgcgacgctgcagca |
| | gtccctctgcagcagatgagcgtgactttggccatttcacgcactcgagtgtacacaatccatttttcttaaagcaaatgactgctgatt |
| | gaccagatactgtaacgctgatttcgctccagatcgcacagatagcgaccatgttgctgcgtctgaaaatctggattccgaattcgacc |
| | ctggcgctccatccatgcaacagatggcgacacttgttacaattcctgtcacccatcggcatggagcaggtccacttagattcccgatc |
| | acccacgcacatctcgctaatagtcattcgttcgtgtcttcgatcaatctcaagtgagtgtgcatggatcttggttgacgatgcggtatg |
| | ggtttgcgccgctggctgcagggtctgcccaaggcaagctaacccagctcctctccccgacaatactctcgcaggcaaagccggtca |
| | cttgccttccagattgccaataaactcaattatggcctctgtcatgccatccatgggtctgatgaatggtcacgctcgtgtcctgaccgtt |
| | 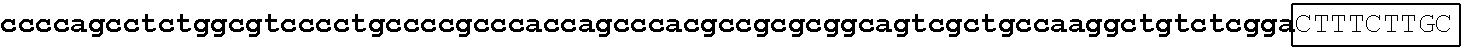 |
| | 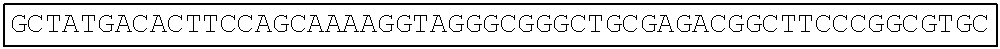 |
| | 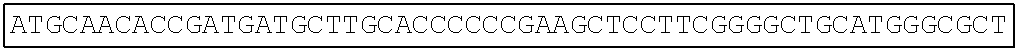 |
| | 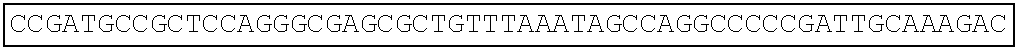 |
| | 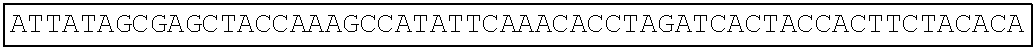 |
| | 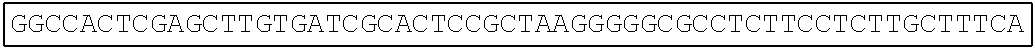 |
| | 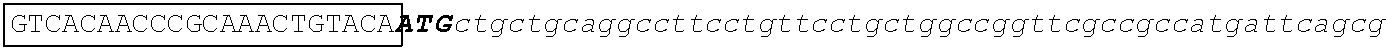 |
| | cctccatgacgaacgagacgtccgaccgccccctggtgcacttcacccccaacaagggctggatgaacgaccccaacggcctgtggta |
| | cgacgagaaggacgccaagtggcacctgtacttccagtacaacccgaacgacaccgtctgggggacgcccttgttctggggccacgcc |
| | acgtccgacgacctgaccaactgggaggaccagcccatcgccatcgccccgaagcgcaacgactccggcgccttctccggctccatgg |
| | tggtggactacaacaacacctccggcttcttcaacgacaccatcgacccgcgccagcgctgcgtggccatctggacctacaacaccccg |
| | gagtccgaggagcagtacatctcctacagcctggacggcggctacaccttcaccgagtaccagaagaaccccgtgctggccgccaact |
| | ccacccagttccgcgacccgaaggtcttctggtacgagccctcccagaagtggatcatgaccgcggccaagtcccaggactacaagat |
| | cgagatctactcctccgacgacctgaagtcctggaagctggagtccgcgttcgccaacgagggcttcctcggctaccagtacgagtgccc |
| | cggcctgatcgaggtccccaccgagcaggaccccagcaagtcctactgggtgatgttcatctccatcaaccccggcgccccggccggc |
| | ggctccttcaaccagtacttcgtcggcagcttcaacggcacccacttcgaggccttcgacaaccagtcccgcgtggtggacttcggcaag |
| | gactactacgccctgcagaccttcttcaacaccgacccgacctacgggagcgccctgggcatcgcgtgggcctccaactgggagtactc |
| | cgccttcgtgcccaccaacccctggcgctcctccatgtccctcgtgcgcaagttctccctcaacaccgagtaccaggccaacccggagac |
| | ggagctgatcaacctgaaggccgagccgatcctgaacatcagcaacgccggcccctggagccggttcgccaccaacaccacgttgac |
| | gaaggccaacagctacaacgtcgacctgtccaacagcaccggcaccctggagttcgagctggtgtacgccgtcaacaccacccagac |
| | gatctccaagtccgtgttcgcggacctctccctctggttcaagggcctggaggaccccgaggagtacctccgcatgggcttcgaggtgtcc |
| | gcgtcctccttcttcctggaccgcgggaacagcaaggtgaagttcgtgaaggagaacccctacttcaccaaccgcatgagcgtgaacaa |
| | ccagcccttcaagagcgagaacgacctgtcctactacaaggtgtacggcttgctggaccagaacatcctggagctgtacttcaacgacg |
| | gcgacgtcgtgtccaccaacacctacttcatgaccaccgggaacgccctgggctccgtgaacatgacgacgggggtggacaacctgttc |
| | tacatcgacaagttccaggtgcgcgaggtcaagtgactTAAGACGCCCGCGCGGCGCACCTGACCTGTTCT |
| | CTCGAGGGCGCCTGTTCTGCCTTGCGAAACAAGCCCCTGGAGCATGCGTGCATGATCG |
| | TCTCTGGCGCCCCGCCGCGCGGTTTGTCGCCCTCGCGGGCGCCGCGGCCGCGGGGGC |
| | GCATTGAAATTGTTGCAAACCCCACCTGACAGATTGAGGGCCCAGGCAGGAAGGCGT |
| | TGAGATGGAGGTACAGGAGTCAAGTAACTGAAAGTTTTTATGATAACTAACAACAAA |
| | GGGTCGTTTCTGGCCAGCGAATGACAAGAACAAGATTCCACATTTCCGTGTAGAGGC |
| | TTGCCATCGAATGTGAGCGGGCGGGCCGCGGACCCGACAAAACCCTTACGACGTGGT |
| | AAGAAAAACGTGGCGGGCACTGTCCCTGTAGCCTGAAGACCAGCAGGAGACGATCG |
| | GAAGCATCACAGCACAACCGGtgcagcagcagctcggatagtatcgacacactctggacgctggtcgtgtgatggact |
| | gttgccgccacacttgctgccttgacctgtgaatatccctgccgcttttatcaaacagcctcagtgtgtttgatcttgtgtgtacgcgcttttgcg |
| | agttgctagctgcttgtgctatttgcgaataccacccccagcatccccttccctcgtttcatatcgcttgcatcccaaccgcaacttatctacgc |
| | tgtcctgctatccctcagcgctgctcctgctcctgctcactgcccctcgcacagccttggtttgggctccgcctgtattctcctggtactgcaacc |
| | 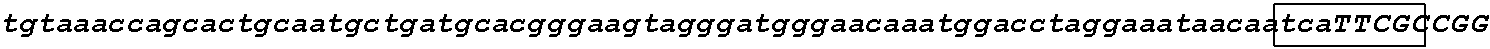 |
| | 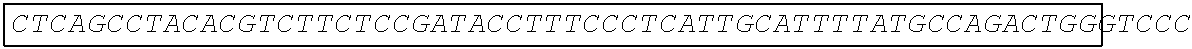 |
| | 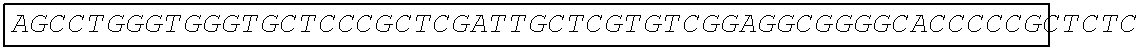 |
| | 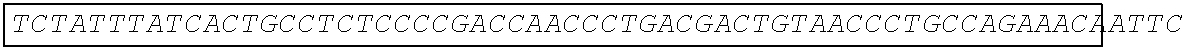 |
| | 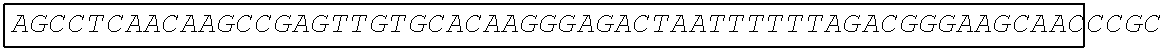 |
| | 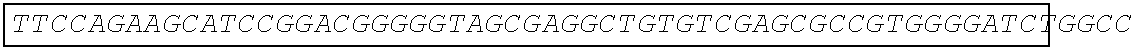 |
| | 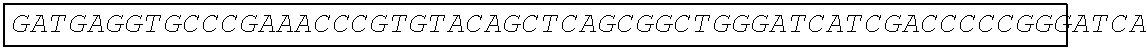 |
| | 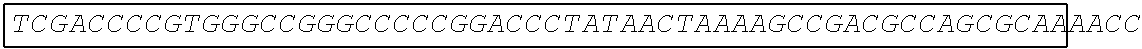 |
| | 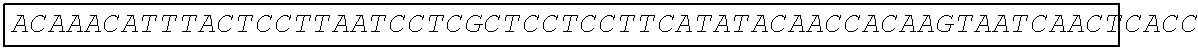 |
| | 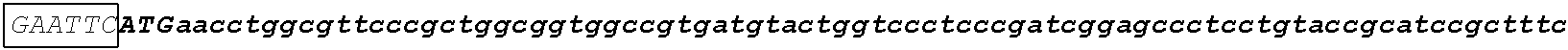 |
| | tcaacgtgttcggcccccgtaacgatatgctggagtgggcggagatgatgcgtgccttcctcggagtgcgcttcaagctcgtgggccaac |
| | gtcgcctgcacaagggcgagcgtatggtgtacctcgccaaccaccgctcgtgggccgactttttcctggacgtgtggacgaccgaggcg |
| | cgctgctcgatcatgtcccgctggatggtcttctttgcctttccggtgtttatggccagcgtcatcgtcctccgcggcatcgtgctgttcaagcg |
| | gggcaagatcaaggacaaggacgcgtttaacgcctggctggacaagaccctggaccgccaggtcaactccggctctctgctcctgtacc |
| | cggaggggacgcgtaacacgaagcctcatgcgctgcctctgaagcgcgggatgctgaagtactcctggagccgcggcctgccattcca |
| | aattatcgtcacgaccaacaaggagcgcgtcctgtcggagaagaagatggcggtcaagttcggggtgacgtgctgtacggggttttccg |
| | aggtcatcgagtccaagcgctacaaggatgacttcgacggctttatggaggaggtccagcgcgtgtgggataaggagtgggcggaggt |
| | gtacaaggccgctgcggagcccgagaagctggaggacctcacgatccccagcccggacgggttcgactacctcccctcgatgaagct |
| | gctccaggtgctggtgaccctcctgaccatcgcggtgttcagcgcgatggtcgtgggctccgcctacgccctgggtctggtggtccccgcg |
| | gcccaggacgcctggaagtacgcgagctacgtcctggcgtcgatgcccatcctgggcctcgccagcaagctgctcgggggcagcaag |
| | gtcggcgctatcaagtatccgggcctcgccagcaagaagattgacTGAcacgcccgcgcggcgcacctgacctgttctctcgagggcg |
| | cctgttctgccttgcgaaacaagcccctggagcatgcgtgcatgatcgtctctggcgccccgccgcgcggtttgtcgccctcgcgggcgccc |
| | cggccgcgggggcgcattgaaattgttgcaaaccccacctgacagattgagggcccaggcaggaaggcgttgagatggaggtacaggagt |
| | caagtaactgaaagtttttatgataactaacaacaaagggtcgtttctggccagcgaatgacaagaacaagattccacatttccgtgtagaggctt |
| | gccatcgaatgtgagcggggggccgcggacccgacaaaacccttacgacgtggtaagaaaaacgtgggggcactgtccctgtagcctg |
| | aagaccagcaggagacgatcggaagcatcacagcacaggccggcccgcgtctcgaacagagcgcggcggccgcatcgatacctgcagg |
| | acagcgccatgccacgccctttgatggcttcaagtacgattacggtgttggattgtgtgtttgttgcgtagtgtgcatggtttagaataat |
| | acacttgatttcttgctcacggcaatctcggcttgtccgcaggttcaaccccatttcggagtctcaggtcagccgcgcaatgaccagcc |
| | gctacttcaaggacttgcacgacaacgccgaggtgagctatgtttaggacttgattggaaattgtcgtcgacgcatattcgcgctccgc |
| | gacagcacccaagcaaaatgtcaagtgcgttccgatttgcgtccgcaggtcgatgttgtgatcgtcggcgccggatccgccggtctgt |
| | cctgcgcttacgagctgaccaagcaccctgacgtccgggtacgcgagctgagattcgattagacataaattgaagattaaacccgta |
| | gaaaaatttgatggtcgcgaaactgtgctcgattgcaagaaattgatcgtcctccactccgcaggtcgccatcatcgagcagggcgtt |
| | gctcccggcggcggcgcctggctggggggacagctgttctcggccatgtgtgtacgtagaaggatgaatttcagctggttttcgttgca |
| | cagctgtttgtgcatgatttgtttcagactattgttgaatgtttttagatttcttaggatgcatgatttgtctgcatgcgact |

The construct can be written as 5′Thi4::CrTUB2:ScSUC2:PmPGH:CvNR:PmG3PDH:PedLPAAT2:PmPGH::3′Thi4. Proceeding in the 5′ to 3′ direction bold, lowercase sequences represent genomic DNA from CHK100 that permit targeted integration at the Thi4 locus via homologous recombination, the C. reinhardtii β-tubulin promoter driving expression of the yeast sucrose invertase gene (conferring the ability of CHK100 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text that enables amplification of the PedLPAAT2 gene, followed by a linker, indicated by lowercase, bold italics, in which contains a C. vulgaris nitrate reductase 3′-UTR. The P. moriformis PmG3PDH promoter, indicated by boxed italicized text, drives the expression of the PedLPAAT2. The initiator ATG and terminator TAG codons of the PedLPAAT2 are indicated by uppercase, bold italics, while the remainder of the gene is indicated by bold italics. The P. moriformis PmPGH 3′-UTR is indicated by lowercase underlined text followed by the CHKC100 Thi4 genomic region indicated by bold, lowercase text.

Table 69 shows the fatty acid/TAG profiles of primary transformants of CHK100 transformed with pCHK1127. The P. moriformis base strain CHKC100 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 69
Screen of primary transformants of CHK100 with pCHK1127.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK1127-1 | 0.03 | 1.23 | 30.12 | 2.18 | 56.67 | 7.51 | 0.45 | 0.18 | 0.07 | 4.64 |
| pCHK1127-2 | 0.03 | 1.25 | 30.36 | 2.41 | 56.39 | 7.34 | 0.44 | 0.19 | 0.07 | 4.15 |
| pCHK1127-3 | 0.03 | 1.24 | 30.80 | 2.33 | 56.06 | 7.31 | 0.45 | 0.17 | 0.07 | 4.49 |
| pCHK1127-4 | 0.03 | 1.19 | 29.75 | 2.32 | 57.15 | 7.36 | 0.43 | 0.18 | 0.07 | 4.86 |
| pCHK1127-5 | 0.03 | 1.27 | 30.60 | 2.33 | 56.14 | 7.36 | 0.45 | 0.18 | 0.07 | 3.9 |
| pCHK1127-6 | 0.03 | 1.40 | 31.48 | 2.46 | 55.45 | 6.87 | 0.45 | 0.20 | 0.05 | 1.65 |
| pCHK1127-9 | 0.03 | 1.25 | 30.64 | 2.28 | 56.14 | 7.41 | 0.44 | 0.18 | 0.08 | 5.16 |
| pCHK1127- | 0.03 | 1.30 | 31.48 | 2.25 | 55.73 | 6.90 | 0.42 | 0.18 | 0.06 | 3.27 |
| 10 | | | | | | | | | | |
| pCHK1127- | 0.03 | 1.21 | 30.09 | 2.28 | 56.78 | 7.39 | 0.44 | 0.18 | 0.07 | 4.89 |
| 11 | | | | | | | | | | |
| pCHK1127- | 0.03 | 1.24 | 30.90 | 2.26 | 56.16 | 7.09 | 0.43 | 0.18 | 0.07 | 3.7 |
| 12 | | | | | | | | | | |
| CHK100 | 0.03 | 1.33 | 32.22 | 2.29 | 55.28 | 6.50 | 0.44 | 0.18 | 0.05 | 1.60 |
| Control | | | | | | | | | | |

### Example 33: Expressing Pedinophyceae sp. LPAAT2 (PedLPAAT2) in P. moriformis Strain CHK100 Using the PmL40-2 Promoter

PedLPAAT2 was introduced into a P. moriformis strain CHK100. The expression construct pCHK1128 contained 5′ and 3′ homology arms to permit targeted integration of PedLPAAT2 into the genome and is shown in Table 70.

TABLE 70
Integrative sequences for the transformation of P. moriformis with pCHK1128
encoding PedLPAAT2.

| SEQ ID | |
|---|---|
| NO: | Nucleotide Sequence |
| 61 | ccctcaactgcgacgctgggaaccttctccgggcaggcgatgtgcgtgggtttgcctccttggcacggctctacaccgtcgagtacgcc |
| | atgaggcggtgatggctgtgtcggttgccacttcgtccagagacggcaagtcgtccatcctctgcgtgtgtggcgcgacgctgcagca |
| | gtccctctgcagcagatgagcgtgactttggccatttcacgcactcgagtgtacacaatccatttttcttaaagcaaatgactgctgatt |
| | gaccagatactgtaacgctgatttcgctccagatcgcacagatagcgaccatgttgctgcgtctgaaaatctggattccgaattcgacc |
| | ctggcgctccatccatgcaacagatggcgacacttgttacaattcctgtcacccatcggcatggagcaggtccacttagattcccgatc |
| | acccacgcacatctcgctaatagtcattcgttcgtgtcttcgatcaatctcaagtgagtgtgcatggatcttggttgacgatgcggtatg |
| | ggtttgcgccgctggctgcagggtctgcccaaggcaagctaacccagctcctctccccgacaatactctcgcaggcaaagccggtca |
| | cttgccttccagattgccaataaactcaattatggcctctgtcatgccatccatgggtctgatgaatggtcacgctcgtgtcctgaccgtt |
| | 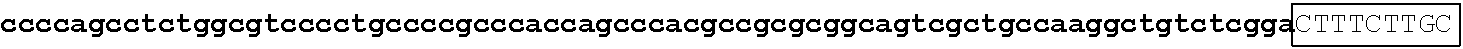 |
| | 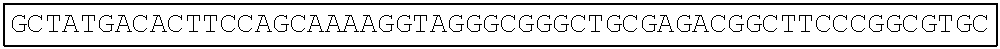 |
| | 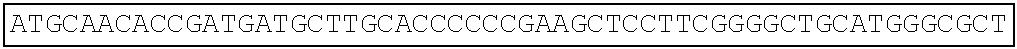 |
| | 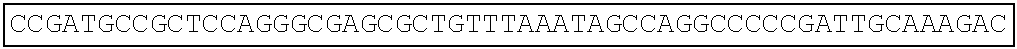 |
| | 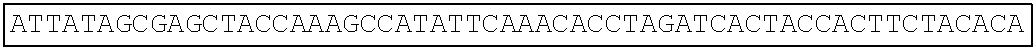 |
| | 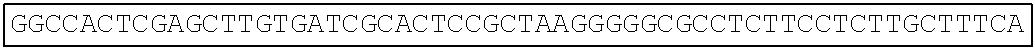 |
| | 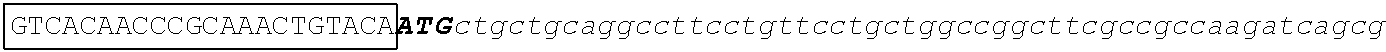 |
| | cctccatgacgaacgagacgtccgaccgccccctggtgcacttcacccccaacaagggctggatgaacgaccccaacggcctgtggta |
| | cgacgagaaggacgccaagtggcacctgtacttccagtacaacccgaacgacaccgtctgggggacgcccttgttctggggccacgcc |
| | acgtccgacgacctgaccaactgggaggaccagcccatcgccatcgccccgaagcgcaacgactccggcgccttctccggctccatgg |
| | tggtggactacaacaacacctccggcttcttcaacgacaccatcgacccgcgccagcgctgcgtggccatctggacctacaacaccccg |
| | gagtccgaggagcagtacatctcctacagcctggacggcggctacaccttcaccgagtaccagaagaaccccgtgctggccgccaact |
| | ccacccagttccgcgacccgaaggtcttctggtacgagccctcccagaagtggatcatgaccgcggccaagtcccaggactacaagat |
| | cgagatctactcctccgacgacctgaagtcctggaagctggagtccgcgttcgccaacgagggcttcctcggctaccagtacgagtgccc |
| | cggcctgatcgaggtccccaccgagcaggaccccagcaagtcctactgggtgatgttcatctccatcaaccccggcgccccggccggc |
| | ggctccttcaaccagtacttcgtcggcagcttcaacggcacccacttcgaggccttcgacaaccagtcccgcgtggtggacttcggcaag |
| | gactactacgccctgcagaccttcttcaacaccgacccgacctacgggagcgccctgggcatcgcgtgggcctccaactgggagtactc |
| | cgccttcgtgcccaccaacccctggcgctcctccatgtccctcgtgcgcaagttctccctcaacaccgagtaccaggccaacccggagac |
| | ggagctgatcaacctgaaggccgagccgatcctgaacatcagcaacgccggcccctggagccggttcgccaccaacaccacgttgac |
| | gaaggccaacagctacaacgtcgacctgtccaacagcaccggcaccctggagttcgagctggtgtacgccgtcaacaccacccagac |
| | gatctccaagtccgtgttcgcggacctctccctctggttcaagggcctggaggaccccgaggagtacctccgcatgggcttcgaggtgtcc |
| | gcgtcctccttcttcctggaccgcgggaacagcaaggtgaagttcgtgaaggagaacccctacttcaccaaccgcatgagcgtgaacaa |
| | ccagcccttcaagagcgagaacgacctgtcctactacaaggtgtacggcttgctggaccagaacatcctggagctgtacttcaacgacg |
| | gcgacgtcgtgtccaccaacacctacttcatgaccaccgggaacgccctgggctccgtgaacatgacgacgggggtggacaacctgttc |
| | tacatcgacaagttccaggtgcgcgaggtcaagtgactTAAGACGCCCGCGCGGCGCACCTGACCTGTTCT |
| | CTCGAGGGCGCCTGTTCTGCCTTGCGAAACAAGCCCCTGGAGCATGCGTGCATGATCG |
| | TCTCTGGCGCCCCGCCGCGCGGTTTGTCGCCCTCGCGGGCGCCGCGGCCGCGGGGGC |
| | GCATTGAAATTGTTGCAAACCCCACCTGACAGATTGAGGGCCCAGGCAGGAAGGCGT |
| | TGAGATGGAGGTACAGGAGTCAAGTAACTGAAAGTTTTTATGATAACTAACAACAAA |
| | GGGTCGTTTCTGGCCAGCGAATGACAAGAACAAGATTCCACATTTCCGTGTAGAGGC |
| | TTGCCATCGAATGTGAGCGGGCGGGCCGCGGACCCGACAAAACCCTTACGACGTGGT |
| | AAGAAAAACGTGGCGGGCACTGTCCCTGTAGCCTGAAGACCAGCAGGAGACGATCG |
| | GAAGCATCACAGCACAACCGGtgcagcagcagctcggatagtatcgacacactctggacgctggtcgtgtgatggact |
| | gttgccgccacacttgctgccttgacctgtgaatatccctgccgcttttatcaaacagcctcagtgtgtttgatcttgtgtgtacgcgcttttgcg |
| | agttgctagctgcttgtgctatttgcgaataccacccccagcatccccttccctcgtttcatatcgcttgcatcccaaccgcaacttatctacgc |
| | tgtcctgctatccctcagcgctgctcctgctcctgctcactgcccctcgcacagccttggtttgggctccgcctgtattctcctggtactgcaacc |
| | 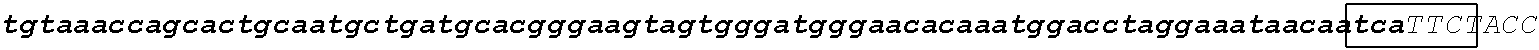 |
| | 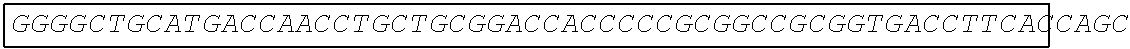 |
| | 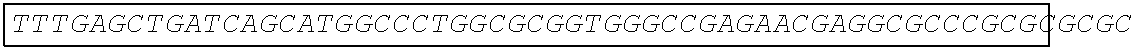 |
| | 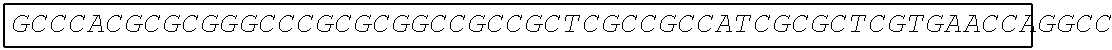 |
| | 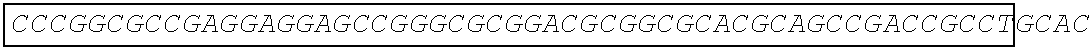 |
| | 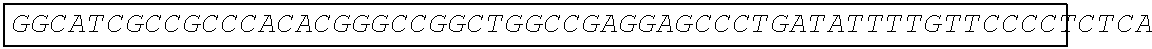 |
| | 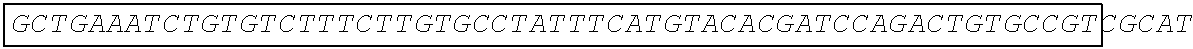 |
| | 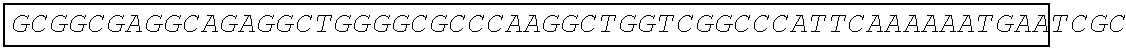 |
| | 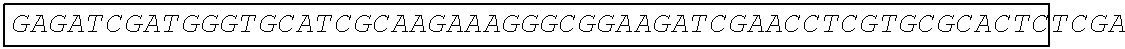 |
| | 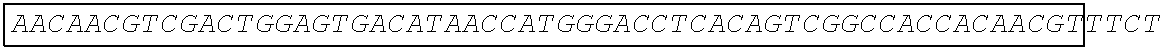 |
| | 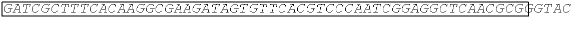 |
| | 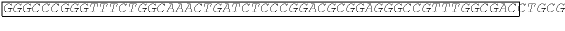 |
| | 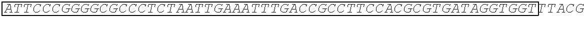 |
| | 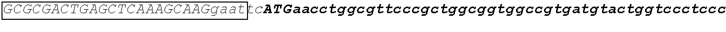 |
| | gatcggagccctcctgtaccgcatccgctttctcaacgtgttcggcccccgtaacgatatgctggagtgggcggagatgatgcgtgccttcc |
| | tcggagtgcgcttcaagctcgtgggccaacgtcgcctgcacaagggcgagcgtatggtgtacctcgccaaccaccgctcgtgggccgac |
| | tttttcctggacgtgtggacgaccgaggcgcgctgctcgatcatgtcccgctggatggtcttctttgcctttccggtgtttatggccagcgtcat |
| | cgtcctccgcggcatcgtgctgttcaagcggggcaagatcaaggacaaggacgcgtttaacgcctggctggacaagaccctggaccgc |
| | caggtcaactccggctctctgctcctgtacccggaggggacgcgtaacacgaagcctcatgcgctgcctctgaagcgcgggatgctgaa |
| | gtactcctggagccgcggcctgccattccaaattatcgtcacgaccaacaaggagcgcgtcctgtcggagaagaagatggcggtcaagt |
| | tcggggtgacgtgctgtacggggttttccgaggtcatcgagtccaagcgctacaaggatgacttcgacggctttatggaggaggtccagc |
| | gcgtgtgggataaggagtgggcggaggtgtacaaggccgctgcggagcccgagaagctggaggacctcacgatccccagcccggac |
| | gggttcgactacctcccctcgatgaagctgctccaggtgctggtgaccctcctgaccatcgcggtgttcagcgcgatggtcgtgggctccg |
| | cctacgccctgggtctggtggtccccgcggcccaggacgcctggaagtacgcgagctacgtcctggcgtcgatgcccatcctgggcctcg |
| | ccagcaagctgctcgggggcagcaaggtcggcgctatcaagtatccgggcctcgccagcaagaagattgacTGAacgcccgcgcg |
| | gcgcacctgacctgttctctcgagggcgcctgttctgccttgcgaaacaagcccctggagcatgcgtgcatgatcgtctctggcgccccgccg |
| | cgcggtttgtcgccctcgcgggcgccccggccgcgggggcgcattgaaattgttgcaaaccccacctgacagattgagggcccaggcagg |
| | aaggcgttgagatggaggtacaggagtcaagtaactgaaagtttttatgataactaacaacaaagggtcgtttctggccagcgaatgacaaga |
| | acaagattccacatttccgtgtagaggcttgccatcgaatgtgagcggggggccgcggacccgacaaaacccttacgacgtggtaagaaa |
| | aacgtggcgggcactgtccctgtagcctgaagaccagcaggagacgatcggaagcatcacagcacaggccggcccgcgtctcgaacaga |
| | gcgcggcggccgcatcgatacctgcaggacagcgccatgccacgccctttgatggcttcaagtacgattacggtgttggattgtgtgttt |
| | gttgcgtagtgtgcatggtttagaataatacacttgatttcttgctcacggcaatctcggcttgtccgcaggttcaaccccatttcggagt |
| | ctcaggtcagccgcgcaatgaccagccgctacttcaaggacttgcacgacaacgccgaggtgagctatgtttaggacttgattggaa |
| | attgtcgtcgacgcatattcgcgctccgcgacagcacccaagcaaaatgtcaagtgcgttccgatttgcgtccgcaggtcgatgttgtg |
| | atcgtcggcgccggatccgccggtctgtcctgcgcttacgagctgaccaagcaccctgacgtccgggtacgcgagctgagattcgatt |
| | agacataaattgaagattaaacccgtagaaaaatttgatggtcgcgaaactgtgctcgattgcaagaaattgatcgtcctccactccg |
| | caggtcgccatcatcgagcagggcgttgctcccggcggcggcgcctggctggggggacagctgttctcggccatgtgtgtacgtaga |
| | aggatgaatttcagctggttttcgttgcacagctgtttgtgcatgatttgtttcagactattgttgaatgtttttagatttcttaggatgcatg |
| | atttgtctgcatgcgact |

The construct can be written as 5′Thi4::CrTUB2:ScSUC2:PmPGH:CvNR:PmL40-2:PedLPAAT2:PmPGH::3′Thi4. Proceeding in the 5′ to 3′ direction bold, lowercase sequences represent genomic DNA from CHK100 that permit targeted integration at the Thi4 locus via homologous recombination, the C. reinhardtii β-tubulin promoter driving expression of the yeast sucrose invertase gene (conferring the ability of CHK100 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text that enables amplification of the PedLPAAT2 gene, followed by a linker, indicated by lowercase, bold italics, in which contains a C. vulgaris nitrate reductase 3′-UTR. The P. moriformis PmL40-2 promoter, indicated by boxed italicized text, drives the expression of the PedLPAAT2. The initiator ATG and terminator TAG codons of the PedLPAAT2 are indicated by uppercase, bold italics, while the remainder of the gene is indicated by bold italics. The P. moriformis PmPGH 3′-UTR is indicated by lowercase underlined text followed by the CHK100 Thi4 genomic region indicated by bold, lowercase text.

Table 71 shows the fatty acid/TAG profiles of primary transformants of CHK100 transformed with pCHK1128. The P. moriformis base strain CHK100 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as

TABLE 71
Screen of primary transformants of CHK100 with pCHK1128.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK1128-1 | 0.04 | 1.41 | 34.8 | 1.97 | 49.82 | 8.93 | 0.76 | 0.23 | 0.1 | 3.36 |
| pCHK1128-2 | 0.03 | 1.19 | 30.92 | 2.12 | 56.26 | 7.13 | 0.44 | 0.17 | 0.07 | 4.03 |
| pCHK1128-3 | 0.03 | 1.17 | 30.62 | 2.16 | 56.48 | 7.21 | 0.43 | 0.18 | 0.07 | 3.7 |
| pCHK1128-4 | 0.03 | 1.16 | 30.83 | 2.12 | 56.42 | 7.13 | 0.44 | 0.17 | 0.07 | 4.1 |
| pCHK1128-5 | 0.03 | 1.15 | 30.44 | 2.18 | 56.87 | 7.02 | 0.45 | 0.17 | 0.07 | 4.1 |
| pCHK1128-6 | 0.03 | 1.17 | 30.56 | 2.2 | 56.56 | 7.17 | 0.44 | 0.18 | 0.07 | 3.88 |
| pCHK1128-7 | 0.03 | 1.18 | 31.27 | 2.05 | 55.99 | 7.1 | 0.43 | 0.16 | 0.06 | 3.92 |
| pCHK1128-8 | 0.03 | 1.19 | 30.09 | 2.34 | 56.76 | 7.36 | 0.44 | 0.18 | 0.07 | 4.69 |
| CHK100 | 0.03 | 1.33 | 32.22 | 2.29 | 55.28 | 6.50 | 0.44 | 0.18 | 0.05 | 1.60 |
| Control | | | | | | | | | | |

### Example 34: Expressing Pedinophyceae sp. LPAAT2 (PedLPAAT2) in P. moriformis Strain CHK100 Using the PmMPGp Promoter

PedLPAAT2 was introduced into a P. moriformis strain CHKC100. The expression construct pCHK1129 contained 5Y and 3Y homology arms to permit targeted integration of PedLPAAT2 into the genome and is shown in Table 72.

TABLE 72
Integrative sequences for the transformation of P. moriformis with pCHK1129
encoding PedLPAAT2.

| SEQ ID | |
|---|---|
| NO: | Nucleotide Sequence |
| 62 | ccctcaactgcgacgctgggaaccttctccgggcaggcgatgtgcgtgggtttgcctccttggcacggctctacaccgtcgagtacgcc |
| | atgaggcggtgatggctgtgtcggttgccacttcgtccagagacggcaagtcgtccatcctctgcgtgtgtggcgcgacgctgcagca |
| | gtccctctgcagcagatgagcgtgactttggccatttcacgcactcgagtgtacacaatccatttttcttaaagcaaatgactgctgatt |
| | gaccagatactgtaacgctgatttcgctccagatcgcacagatagcgaccatgttgctgcgtctgaaaatctggattccgaattcgacc |
| | ctggcgctccatccatgcaacagatggcgacacttgttacaattcctgtcacccatcggcatggagcaggtccacttagattcccgatc |
| | acccacgcacatctcgctaatagtcattcgttcgtgtcttcgatcaatctcaagtgagtgtgcatggatcttggttgacgatgcggtatg |
| | ggtttgcgccgctggctgcagggtctgcccaaggcaagctaacccagctcctctccccgacaatactctcgcaggcaaagccggtca |
| | cttgccttccagattgccaataaactcaattatggcctctgtcatgccatccatgggtctgatgaatggtcacgctcgtgtcctgaccgtt |
| | 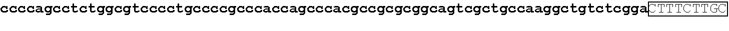 |
| | 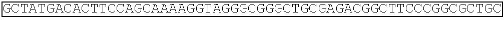 |
| | 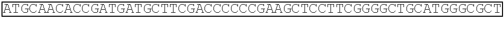 |
| | 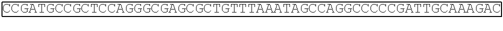 |
| | 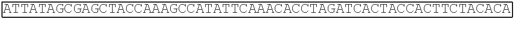 |
| | 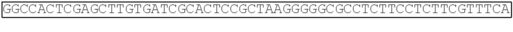 |
| | 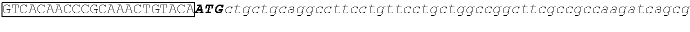 |
| | cctccatgacgaacgagacgtccgaccgccccctggtgcacttcacccccaacaagggctggatgaacgaccccaacggcctgtggta |
| | cgacgagaaggacgccaagtggcacctgtacttccagtacaacccgaacgacaccgtctgggggacgcccttgttctggggccacgcc |
| | acgtccgacgacctgaccaactgggaggaccagcccatcgccatcgccccgaagcgcaacgactccggcgccttctccggctccatgg |
| | tggtggactacaacaacacctccggcttcttcaacgacaccatcgacccgcgccagcgctgcgtggccatctggacctacaacaccccg |
| | gagtccgaggagcagtacatctcctacagcctggacggcggctacaccttcaccgagtaccagaagaaccccgtgctggccgccaact |
| | ccacccagttccgcgacccgaaggtcttctggtacgagccctcccagaagtggatcatgaccgcggccaagtcccaggactacaagat |
| | cgagatctactcctccgacgacctgaagtcctggaagctggagtccgcgttcgccaacgagggcttcctcggctaccagtacgagtgccc |
| | cggcctgatcgaggtccccaccgagcaggaccccagcaagtcctactgggtgatgttcatctccatcaaccccggcgccccggccggc |
| | ggctccttcaaccagtacttcgtcggcagcttcaacggcacccacttcgaggccttcgacaaccagtcccgcgtggtggacttcggcaag |
| | gactactacgccctgcagaccttcttcaacaccgacccgacctacgggagcgccctgggcatcgcgtgggcctccaactgggagtactc |
| | cgccttcgtgcccaccaacccctggcgctcctccatgtccctcgtgcgcaagttctccctcaacaccgagtaccaggccaacccggagac |
| | ggagctgatcaacctgaaggccgagccgatcctgaacatcagcaacgccggcccctggagccggttcgccaccaacaccacgttgac |
| | gaaggccaacagctacaacgtcgacctgtccaacagcaccggcaccctggagttcgagctggtgtacgccgtcaacaccacccagac |
| | gatctccaagtccgtgttcgcggacctctccctctggttcaagggcctggaggaccccgaggagtacctccgcatgggcttcgaggtgtcc |
| | gcgtcctccttcttcctggaccgcgggaacagcaaggtgaagttcgtgaaggagaacccctacttcaccaaccgcatgagcgtgaacaa |
| | ccagcccttcaagagcgagaacgacctgtcctactacaaggtgtacggcttgctggaccagaacatcctggagctgtacttcaacgacg |
| | gcgacgtcgtgtccaccaacacctacttcatgaccaccgggaacgccctgggctccgtgaacatgacgacgggggtggacaacctgttc |
| | tacatcgacaagttccaggtgcgcgaggtcaagtgactTAAGACGCCCGCGCGGCGCACCTGACCTGTTCT |
| | CTCGAGGGCGCCTGTTCTGCCTTGCGAAACAAGCCCCTGGAGCATGCGTGCATGATCG |
| | TCTCTGGCGCCCCGCCGCGCGGTTTGTCGCCCTCGCGGGCGCCGCGGCCGCGGGGGC |
| | GCATTGAAATTGTTGCAAACCCCACCTGACAGATTGAGGGCCCAGGCAGGAAGGCGT |
| | TGAGATGGAGGTACAGGAGTCAAGTAACTGAAAGTTTTTATGATAACTAACAACAAA |
| | GGGTCGTTTCTGGCCAGCGAATGACAAGAACAAGATTCCACATTTCCGTGTAGAGGC |
| | TTGCCATCGAATGTGAGCGGGCGGGCCGCGGACCCGACAAAACCCTTACGACGTGGT |
| | AAGAAAAACGTGGCGGGCACTGTCCCTGTAGCCTGAAGACCAGCAGGAGACGATCG |
| | GAAGCATCACAGCACAACCGGtgcagcagcagctcggatagtatcgacacactctggacgctggtcgtgtgatggact |
| | gttgccgccacacttgctgccttgacctgtgaatatccctgccgcttttatcaaacagcctcagtgtgtttgatcttgtgtgtacgcgcttttgcg |
| | agttgctagctgcttgtgctatttgcgaataccacccccagcatccccttccctcgtttcatatcgcttgcatcccaaccgcaacttatctacgc |
| | tgtcctgctatccctcagcgctgctcctgctcctgctcactgcccctcgcacagccttggtttgggctccgcctgtattctcctggtactgcaacc |
| | 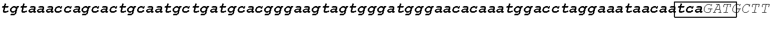 |
| | 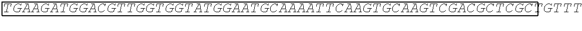 |
| | 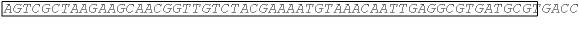 |
| | 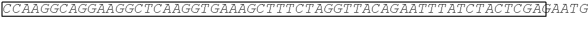 |
| | 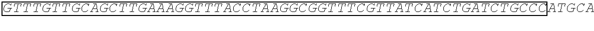 |
| | 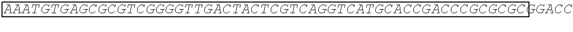 |
| | 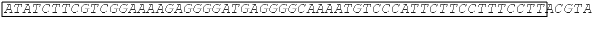 |
| | 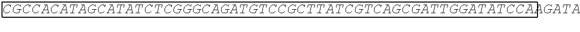 |
| | 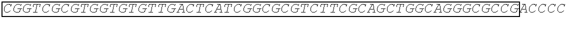 |
| | 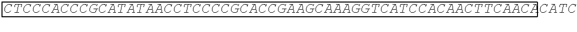 |
| | 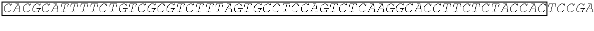 |
| | 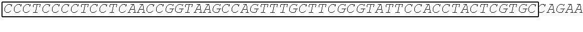 |
| | 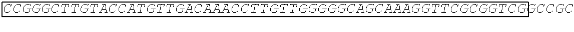 |
| | 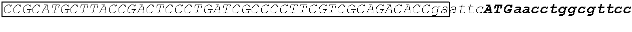 |
| | cgctggcggtggccgtgatgtactggtccctcccgatcggagccctcctgtaccgcatccgctttctcaacgtgttcggcccccgtaacgat |
| | atgctggagtgggcggagatgatgcgtgccttcctcggagtgcgcttcaagctcgtgggccaacgtcgcctgcacaagggcgagcgtat |
| | ggtgtacctcgccaaccaccgctcgtgggccgactttttcctggacgtgtggacgaccgaggcgcgctgctcgatcatgtcccgctggatg |
| | gtcttctttgcctttccggtgtttatggccagcgtcatcgtcctccgcggcatcgtgctgttcaagcggggcaagatcaaggacaaggacgc |
| | gtttaacgcctggctggacaagaccctggaccgccaggtcaactccggctctctgctcctgtacccggaggggacgcgtaacacgaagc |
| | ctcatgcgctgcctctgaagcgcgggatgctgaagtactcctggagccgcggcctgccattccaaattatcgtcacgaccaacaaggag |
| | cgcgtcctgtcggagaagaagatggcggtcaagttcggggtgacgtgctgtacggggttttccgaggtcatcgagtccaagcgctacaa |
| | ggatgacttcgacggctttatggaggaggtccagcgcgtgtgggataaggagtgggcggaggtgtacaaggccgctgcggagcccga |
| | gaagctggaggacctcacgatccccagcccggacgggttcgactacctcccctcgatgaagctgctccaggtgctggtgaccctcctga |
| | ccatcgcggtgttcagcgcgatggtcgtgggctccgcctacgccctgggtctggtggtccccgcggcccaggacgcctggaagtacgcg |
| | agctacgtcctggcgtcgatgcccatcctgggcctcgccagcaagctgctcgggggcagcaaggtcggcgctatcaagtatccgggcct |
| | cgccagcaagaagattgacTGAacgcccgcgcggcgcacctgacctgttctctcgagggcgcctgttctgccttgcgaaacaagcccct |
| | ggagcatgcgtgcatgatcgtctctggcgccccgccgcgcggtttgtcgccctcgcgggcgccccggccgcgggggcgcattgaaattgtt |
| | gcaaaccccacctgacagattgagggcccaggcaggaaggcgttgagatggaggtacaggagtcaagtaactgaaagtttttatgataacta |
| | acaacaaagggtcgtttctggccagcgaatgacaagaacaagattccacatttccgtgtagaggcttgccatcgaatgtgagcggggggcc |
| | gcggacccgacaaaacccttacgacgtggtaagaaaaacgtgggggcactgtccctgtagcctgaagaccagcaggagacgatcggaa |
| | gcatcacagcacaggccggcccgcgtctcgaacagagcgcggcggccgcatcgatacctgcaggacagcgccatgccacgccctttga |
| | tggcttcaagtacgattacggtgttggattgtgtgtttgttgcgtagtgtgcatggtttagaataatacacttgatttcttgctcacggcaa |
| | tctcggcttgtccgcaggttcaaccccatttcggagtctcaggtcagccgcgcaatgaccagccgctacttcaaggacttgcacgaca |
| | acgccgaggtgagctatgtttaggacttgattggaaattgtcgtcgacgcatattcgcgctccgcgacagcacccaagcaaaatgtca |
| | agtgcgttccgatttgcgtccgcaggtcgatgttgtgatcgtcggcgccggatccgccggtctgtcctgcgcttacgagctgaccaagc |
| | accctgacgtccgggtacgcgagctgagattcgattagacataaattgaagattaaacccgtagaaaaatttgatggtcgcgaaact |
| | gtgctcgattgcaagaaattgatcgtcctccactccgcaggtcgccatcatcgagcagggcgttgctcccggcggcggcgcctggctg |
| | gggggacagctgttctcggccatgtgtgtacgtagaaggatgaatttcagctggttttcgttgcacagctgtttgtgcatgatttgtttca |
| | gactattgttgaatgtttttagatttcttaggatgcatgatttgtctgcatgcgact |

The construct can be written as 5′Thi4::CrTUB2:ScSUC2:PmPGH:CvNR:PmMPGp:PedLAAT2:PmPGH::3′Thi4. Proceeding in the 5Y to 3Y direction, bold, lowercase sequences represent genomic DNA from CHK100 that permit targeted integration at the Thi4 locus via homologous recombination, the C. reinhardtii β-tubulin promoter driving expression of the yeast sucrose invertase gene (conferring the ability of CHK100 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text that enables amplification of the PedLPAAT2 gene, followed by a linker, indicated by lowercase, bold italics, in which contains a C. vulgaris nitrate reductase 3′-UTR. The P. moriformis PmMPGp promoter, indicated by boxed italicized text, drives the expression of the PedLPAAT2. The initiator ATG and terminator TAG codons of the PedLPAAT2 are indicated by uppercase, bold italics, while the remainder of the gene is indicated by bold italics. The P. moriformis PmPGH 3′-UTR is indicated by lowercase underlined text followed by the CHK100 Thi4 genomic region indicated by bold, lowercase text.

Table 73 shows the fatty acid/TAG profiles of primary transformants of CHK100 transformed with pCHK1129. The P. moriformis base strain CHK100 is shown as a non-transgenic control. Strains were grown for 96 hours in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 73
Screen of primary transformants of CHK100 with pCHK1129.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK1129-1 | 0.03 | 1.43 | 29.85 | 2.41 | 58.08 | 6 | 0.41 | 0.18 | 0.06 | 4.42 |
| pCHK1129-2 | 0.03 | 1.15 | 31.01 | 2.04 | 56.4 | 7 | 0.43 | 0.16 | 0.06 | 4.3 |
| pCHK1129-3 | 0.03 | 1.17 | 31.42 | 2.08 | 56.17 | 6.74 | 0.42 | 0.16 | 0.06 | 3 |
| pCHK1129-4 | 0.03 | 1.41 | 32.42 | 2.2 | 53.58 | 7.95 | 0.44 | 0.19 | 0.07 | 4.67 |
| pCHK1129-5 | 0.04 | 1.58 | 31.55 | 2.03 | 55.55 | 6.8 | 0.49 | 0.16 | 0.06 | 4.42 |
| pCHK1129-6 | 0.03 | 1.04 | 39.32 | 1.62 | 48.62 | 6.57 | 0.35 | 0.13 | 0.06 | 4.08 |
| pCHK1129-7 | 0.03 | 1.23 | 32.2 | 2.05 | 55.64 | 6.38 | 0.42 | 0.16 | 0.05 | 1.57 |
| pCHK1129-8 | 0.04 | 1.39 | 35.13 | 1.84 | 51.11 | 7.73 | 0.43 | 0.22 | 0.05 | 1.46 |
| CHK100 | 0.03 | 1.33 | 32.22 | 2.29 | 55.28 | 6.50 | 0.44 | 0.18 | 0.05 | 1.60 |
| Control | | | | | | | | | | |

### Example 35: Expressing Volvulina compacta (VcomLPAAT2) in P. moriformis Strain CHK22 Using the PmG3PDH Promoter

VcomLPAAT2 was introduced into a P. moriformis strain CHK22. The expression construct pCHK1210 contained 5Y and 3Y homology arms to permit targeted integration of VcomLPAAT2 into the genome and is shown in Table 74.

TABLE 74
Integrative sequences for the transformation of P. moriformis with pCHK1210
encoding VcomLPAAT2.

| SEQ ID | |
|---|---|
| NO: | Nucleotide Sequence |
| 65 | ccctcaactgcgacgctgggaaccttctccgggcaggcgatgtgcgtgggtttgcctccttggcacggctctacaccgtcgagtacgcc |
| | atgaggcggtgatggctgtgtcggttgccacttcgtccagagacggcaagtcgtccatcctctgcgtgtgtggcgcgacgctgcagca |
| | gtccctctgcagcagatgagcgtgactttggccatttcacgcactcgagtgtacacaatccatttttcttaaagcaaatgactgctgatt |
| | gaccagatactgtaacgctgatttcgctccagatcgcacagatagcgaccatgttgctgcgtctgaaaatctggattccgaattcgacc |
| | ctggcgctccatccatgcaacagatggcgacacttgttacaattcctgtcacccatcggcatggagcaggtccacttagattcccgatc |
| | acccacgcacatctcgctaatagtcattcgttcgtgtcttcgatcaatctcaagtgagtgtgcatggatcttggttgacgatgcggtatg |
| | ggtttgcgccgctggctgcagggtctgcccaaggcaagctaacccagctcctctccccgacaatactctcgcaggcaaagccggtca |
| | cttgccttccagattgccaataaactcaattatggcctctgtcatgccatccatgggtctgatgaatggtcacgctcgtgtcctgaccgtt |
| | 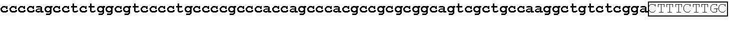 |
| | 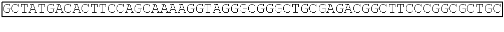 |
| | 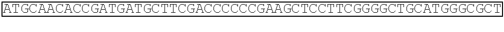 |
| | 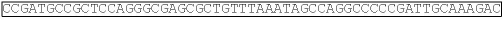 |
| | 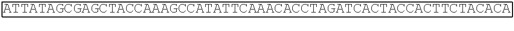 |
| | 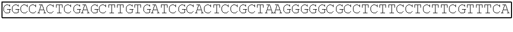 |
| | 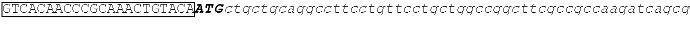 |
| | cctccatgacgaacgagacgtccgaccgccccctggtgcacttcacccccaacaagggctggatgaacgaccccaacggcctgtggta |
| | cgacgagaaggacgccaagtggcacctgtacttccagtacaacccgaacgacaccgtctgggggacgcccttgttctggggccacgcc |
| | acgtccgacgacctgaccaactgggaggaccagcccatcgccatcgccccgaagcgcaacgactccggcgccttctccggctccatgg |
| | tggtggactacaacaacacctccggcttcttcaacgacaccatcgacccgcgccagcgctgcgtggccatctggacctacaacaccccg |
| | gagtccgaggagcagtacatctcctacagcctggacggcggctacaccttcaccgagtaccagaagaaccccgtgctggccgccaact |
| | ccacccagttccgcgacccgaaggtcttctggtacgagccctcccagaagtggatcatgaccgcggccaagtcccaggactacaagat |
| | cgagatctactcctccgacgacctgaagtcctggaagctggagtccgcgttcgccaacgagggcttcctcggctaccagtacgagtgccc |
| | cggcctgatcgaggtccccaccgagcaggaccccagcaagtcctactgggtgatgttcatctccatcaaccccggcgccccggccggc |
| | ggctccttcaaccagtacttcgtcggcagcttcaacggcacccacttcgaggccttcgacaaccagtcccgcgtggtggacttcggcaag |
| | gactactacgccctgcagaccttcttcaacaccgacccgacctacgggagcgccctgggcatcgcgtgggcctccaactgggagtactc |
| | cgccttcgtgcccaccaacccctggcgctcctccatgtccctcgtgcgcaagttctccctcaacaccgagtaccaggccaacccggagac |
| | ggagctgatcaacctgaaggccgagccgatcctgaacatcagcaacgccggcccctggagccggttcgccaccaacaccacgttgac |
| | gaaggccaacagctacaacgtcgacctgtccaacagcaccggcaccctggagttcgagctggtgtacgccgtcaacaccacccagac |
| | gatctccaagtccgtgttcgcggacctctccctctggttcaagggcctggaggaccccgaggagtacctccgcatgggcttcgaggtgtcc |
| | gcgtcctccttcttcctggaccgcgggaacagcaaggtgaagttcgtgaaggagaacccctacttcaccaaccgcatgagcgtgaacaa |
| | ccagcccttcaagagcgagaacgacctgtcctactacaaggtgtacggcttgctggaccagaacatcctggagctgtacttcaacgacg |
| | gcgacgtcgtgtccaccaacacctacttcatgaccaccgggaacgccctgggctccgtgaacatgacgacgggggtggacaacctgttc |
| | tacatcgacaagttccaggtgcgcgaggtcaagtgactTAAGACGCCCGCGCGGCGCACCTGACCTGTTCT |
| | CTCGAGGGCGCCTGTTCTGCCTTGCGAAACAAGCCCCTGGAGCATGCGTGCATGATCG |
| | TCTCTGGCGCCCCGCCGCGCGGTTTGTCGCCCTCGCGGGCGCCGCGGCCGCGGGGGC |
| | GCATTGAAATTGTTGCAAACCCCACCTGACAGATTGAGGGCCCAGGCAGGAAGGCGT |
| | TGAGATGGAGGTACAGGAGTCAAGTAACTGAAAGTTTTTATGATAACTAACAACAAA |
| | GGGTCGTTTCTGGCCAGCGAATGACAAGAACAAGATTCCACATTTCCGTGTAGAGGC |
| | TTGCCATCGAATGTGAGCGGGCGGGCCGCGGACCCGACAAAACCCTTACGACGTGGT |
| | AAGAAAAACGTGGCGGGCACTGTCCCTGTAGCCTGAAGACCAGCAGGAGACGATCG |
| | GAAGCATCACAGCACAACCGGTgcagcagcagctcggatagtatcgacacactctggacgctggtcgtgtgatggac |
| | tgttgccgccacacttgctgccttgacctgtgaatatccctgccgcttttatcaaacagcctcagtgtgtttgatcttgtgtgtacgcgcttttgcg |
| | agttgctagctgcttgtgctatttgcgaataccacccccagcatccccttccctcgtttcatatcgcttgcatcccaaccgcaacttatctacgc |
| | tgtcctgctatccctcagcgctgctcctgctcctgctcactgcccctcgcacagccttggtttgggctccgcctgtattctcctggtactgcaacc |
| | 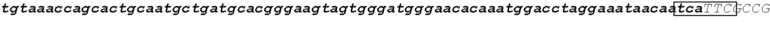 |
| | 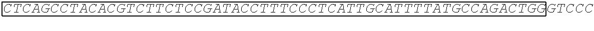 |
| | 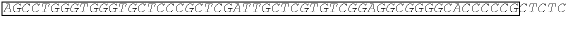 |
| | 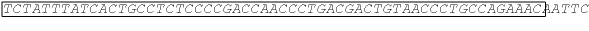 |
| | 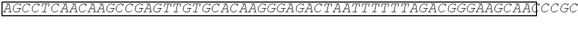 |
| | 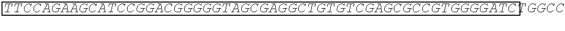 |
| | 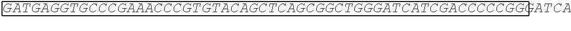 |
| | 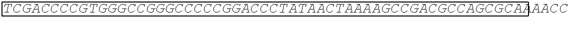 |
| | 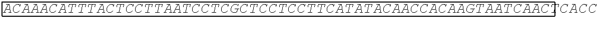 |
| | 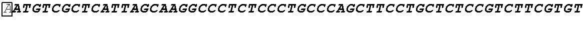 |
| | TCTACTGGTCGCTGCCGATCTTCGCCATCCCCTACCGCATCCGGTTCGCCAACGTCGGC |
| | AAGCGGAACGACATGCTCGACTGGGCGAAGGCTCTGTTCGCGTTTTTCCGCGTCACCGT |
| | CCTGAAGCAGGGGGAGCAGGGCCTCTACCGCGGTGGCCCATGCCTGTACCTGTGCAAC |
| | CACCGCTCGTGGGCGGACTTCTTTGTGGACGTCTACCTGACGGAGGGCCGGGCCGCGC |
| | TGATGTCCCGCTGGCTGGTGTTTTTCGTCTTCCCTGTGTTCTGCACCTCCTGCCTCATCC |
| | TGCGGGGCATCGTGCTGTTCAAGCGCGGGACCATCCTCGACAAGGAGGCCTTTAACGC |
| | GTGGCTCGACGCCACGCTGCGGCGCTCCACCGTCCCCGGCCTGCTCATTTACCCCGAG |
| | GGGCACCGCTCGCTGAAGCCGACCTCGCTGCCGCTGAAGCGGGGCATGCTGCACTTCG |
| | CTTACAGCCGACGGCTCCCCGTCCAACTGATCGTGACCCGCGGCAAGGAGGACATCCT |
| | GTCGGAGAAGACGATGGCCATGAAGTACGGCCGCACCCTCGTGACCACGTACTCGAAG |
| | GTCCTGAAGCCCACGGACTACGACAGCTTCGACGCCTTCATGGGTGACGTCCAGACGA |
| | GCTGGGACGCGGGCTGGAAGGCCTCCTACGAGGCCCCCACCAAGGGGGTGCCCCAACT |
| | GTCCATGGCGGGTGCCCACGAGTACGACTACCCGGCGACCATGCGGTGGCAGCAACTG |
| | GCGATCACCCTCTTCTCGATCGTCGTGTTCGCGGCCGTGCTGTACGGCACGTGGTGGG |
| | GCCTGGGTGCCGTCCTGGCTGCGGCTGGCCCCCTCGGCCAGCAAGTGGTCGTGGTCCT |
| | GGCGGCCACCTCCTGGCTGGGCGCCAGCCTGATCCGGTCCTGCCGCTAGgtaccacgcccgcg |
| | cggcgcacctgacctgttctctcgagggcgcctgttctgccttgcgaaacaagcccctggagcatgcgtgcatgatcgtctctggcgccccgc |
| | cgcgcggtttgtcgccctcgcgggcgccccggccgcgggggcgcattgaaattgttgcaaaccccacctgacagattgagggcccaggca |
| | ggaaggcgttgagatggaggtacaggagtcaagtaactgaaagtttttatgataactaacaacaaagggtcgtttctggccagcgaatgacaa |
| | gaacaagattccacatttccgtgtagaggcttgccatcgaatgtgagcggggggccgcggacccgacaaaacccttacgacgtggtaaga |
| | aaaacgtgggggcactgtccctgtagcctgaagaccagcaggagacgatoggaagcatcacagcacaggccggcccgcgtctcgaaca |
| | gagcgcggcggccgcatcgatacctgcaggacagcgccatgccacgccctttgatggcttcaagtacgattacggtgttggattgtgtg |
| | tttgttgcgtagtgtgcatggtttagaataatacacttgatttcttgctcacggcaatctcggcttgtccgcaggttcaaccccatttcgga |
| | gtctcaggtcagccgcgcaatgaccagccgctacttcaaggacttgcacgacaacgccgaggtgagctatgtttaggacttgattgga |
| | aattgtcgtcgacgcatattcgcgctccgcgacagcacccaagcaaaatgtcaagtgcgttccgatttgcgtccgcaggtcgatgttgt |
| | gatcgtcggcgccggatccgccggtctgtcctgcgcttacgagctgaccaagcaccctgacgtccgggtacgcgagctgagattcgat |
| | tagacataaattgaagattaaacccgtagaaaaatttgatggtcgcgaaactgtgctcgattgcaagaaattgatcgtcctccactcc |
| | gcaggtcgccatcatcgagcagggcgttgctcccggcggcggcgcctggctggggggacagctgttctcggccatgtgtgtacgtag |
| | aaggatgaatttcagctggttttcgttgcacagctgtttgtgcatgatttgtttcagactattgttgaatgtttttagatttcttaggatgcat |
| | gatttgtctgcatgcgact |

The construct can be written as 5′Thia4::CrTUB2:ScSUC2:PmPGH:CvNR:PmG3PDH:VcomLPAAT2:PmPGH::3′Thia4. Proceeding in the 5′ to 3′ direction, bold, lowercase sequences represent genomic DNA from CHK22 that permit targeted integration at the Thia4 locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR that enables amplification of the VcomLPAAT2 gene, is indicated by uppercase underlined text followed by a linker, indicated by lowercase, bold italics, which contains a C. vulgaris nitrate reductase 3′-UTR. The P. moriformis PmG3PDH promoter, indicated by boxed italicized text, drives the expression of the VcomLPAAT2. The initiator ATG and terminator TAG codons of the VcomLPAAT2 are indicated by uppercase, bold italics, while the remainder of the gene is indicated by bold italics. The PmPGH 3′-UTR is indicated by lowercase underlined text followed by the CHK22 Thia4 genomic region indicated by bold, lowercase text.

Table 75 shows the fatty acid/TAG profiles of primary transformants of CHK22 transformed with pCHK1210. The P. moriformis base strain CHK22 is shown as a non-transgenic control. Strains were grown for 96 hrs in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 75
Screen of primary transformants of CHK22 with pCHK1210.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK1210-1 | 0.04 | 1.34 | 27.76 | 2.47 | 57.59 | 8.42 | 0.51 | 0.22 | 0.00 | 4.25 |
| pCHK1210-2 | 0.04 | 1.34 | 27.91 | 2.78 | 57.65 | 7.96 | 0.51 | 0.25 | 0.00 | 3.93 |
| pCHK1210-3 | 0.03 | 1.30 | 27.68 | 2.63 | 58.35 | 7.58 | 0.48 | 0.23 | 0.00 | 4.24 |
| pCHK1210-4 | 0.04 | 1.29 | 27.75 | 2.60 | 58.19 | 7.69 | 0.49 | 0.23 | 0.00 | 3.75 |
| pCHK1210-5 | 0.04 | 1.37 | 28.03 | 2.42 | 57.05 | 8.60 | 0.49 | 0.23 | 0.00 | 4.39 |
| pCHK1210-6 | 0.04 | 1.34 | 28.03 | 2.75 | 57.66 | 7.72 | 0.50 | 0.24 | 0.00 | 3.69 |
| pCHK1210-7 | 0.04 | 1.32 | 27.80 | 2.66 | 57.78 | 7.91 | 0.51 | 0.23 | 0.00 | 3.97 |
| pCHK1210-8 | 0.04 | 1.34 | 28.09 | 2.83 | 57.40 | 7.87 | 0.51 | 0.25 | 0.00 | 3.94 |
| CHK22 | 0.04 | 1.27 | 27.29 | 2.45 | 59.08 | 7.45 | 0.54 | 0.23 | 0.00 | 1.5 |
| Control | | | | | | | | | | |

### Example 36: Expressing Vitreochlamys sp. CL-2021 (VitrLPAAT2) in P. moriformis Strain CHK22 Using the PmG3PDH Promoter

VitrLPAAT2 was introduced into a P. moriformis strain CHK22. The expression construct pCHK1211 contained 5Y and 3Y homology arms to permit targeted integration of VitrLPAAT2 into the genome and is shown in Table 76.

TABLE 76
Integrative sequences for the transformation of P. moriformis with pCHK1211
encoding VitrLPAAT2.

| SEQ ID | |
|---|---|
| NO: | Nucleotide Sequence |
| 66 | ccctcaactgcgacgctgggaaccttctccgggcaggcgatgtgcgtgggtttgcctccttggcacggctctacaccgtcgagtacgcc |
| | atgaggcggtgatggctgtgtcggttgccacttcgtccagagacggcaagtcgtccatcctctgcgtgtgtggcgcgacgctgcagca |
| | gtccctctgcagcagatgagcgtgactttggccatttcacgcactcgagtgtacacaatccatttttcttaaagcaaatgactgctgatt |
| | gaccagatactgtaacgctgatttcgctccagatcgcacagatagcgaccatgttgctgcgtctgaaaatctggattccgaattcgacc |
| | ctggcgctccatccatgcaacagatggcgacacttgttacaattcctgtcacccatcggcatggagcaggtccacttagattcccgatc |
| | acccacgcacatctcgctaatagtcattcgttcgtgtcttcgatcaatctcaagtgagtgtgcatggatcttggttgacgatgcggtatg |
| | ggtttgcgccgctggctgcagggtctgcccaaggcaagctaacccagctcctctccccgacaatactctcgcaggcaaagccggtca |
| | cttgccttccagattgccaataaactcaattatggcctctgtcatgccatccatgggtctgatgaatggtcacgctcgtgtcctgaccgtt |
| | 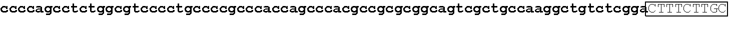 |
| | 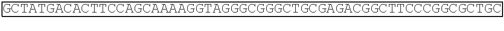 |
| | 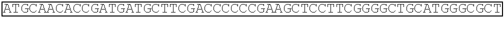 |
| | 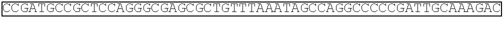 |
| | 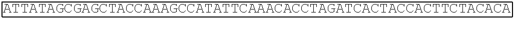 |
| | 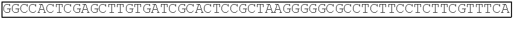 |
| | 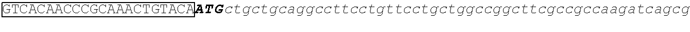 |
| | cctccatgacgaacgagacgtccgaccgccccctggtgcacttcacccccaacaagggctggatgaacgaccccaacggcctgtggta |
| | cgacgagaaggacgccaagtggcacctgtacttccagtacaacccgaacgacaccgtctgggggacgcccttgttctggggccacgcc |
| | acgtccgacgacctgaccaactgggaggaccagcccatcgccatcgccccgaagcgcaacgactccggcgccttctccggctccatgg |
| | tggtggactacaacaacacctccggcttcttcaacgacaccatcgacccgcgccagcgctgcgtggccatctggacctacaacaccccg |
| | gagtccgaggagcagtacatctcctacagcctggacggcggctacaccttcaccgagtaccagaagaaccccgtgctggccgccaact |
| | ccacccagttccgcgacccgaaggtcttctggtacgagccctcccagaagtggatcatgaccgcggccaagtcccaggactacaagat |
| | cgagatctactcctccgacgacctgaagtcctggaagctggagtccgcgttcgccaacgagggcttcctcggctaccagtacgagtgccc |
| | cggcctgatcgaggtccccaccgagcaggaccccagcaagtcctactgggtgatgttcatctccatcaaccccggcgccccggccggc |
| | ggctccttcaaccagtacttcgtcggcagcttcaacggcacccacttcgaggccttcgacaaccagtcccgcgtggtggacttcggcaag |
| | gactactacgccctgcagaccttcttcaacaccgacccgacctacgggagcgccctgggcatcgcgtgggcctccaactgggagtactc |
| | cgccttcgtgcccaccaacccctggcgctcctccatgtccctcgtgcgcaagttctccctcaacaccgagtaccaggccaacccggagac |
| | ggagctgatcaacctgaaggccgagccgatcctgaacatcagcaacgccggcccctggagccggttcgccaccaacaccacgttgac |
| | gaaggccaacagctacaacgtcgacctgtccaacagcaccggcaccctggagttcgagctggtgtacgccgtcaacaccacccagac |
| | gatctccaagtccgtgttcgcggacctctccctctggttcaagggcctggaggaccccgaggagtacctccgcatgggcttcgaggtgtcc |
| | gcgtcctccttcttcctggaccgcgggaacagcaaggtgaagttcgtgaaggagaacccctacttcaccaaccgcatgagcgtgaacaa |
| | ccagcccttcaagagcgagaacgacctgtcctactacaaggtgtacggcttgctggaccagaacatcctggagctgtacttcaacgacg |
| | gcgacgtcgtgtccaccaacacctacttcatgaccaccgggaacgccctgggctccgtgaacatgacgacgggggtggacaacctgttc |
| | tacatcgacaagttccaggtgcgcgaggtcaagtgactTAAGACGCCCGCGCGGCGCACCTGACCTGTTCT |
| | CTCGAGGGCGCCTGTTCTGCCTTGCGAAACAAGCCCCTGGAGCATGCGTGCATGATCG |
| | TCTCTGGCGCCCCGCCGCGCGGTTTGTCGCCCTCGCGGGCGCCGCGGCCGCGGGGGC |
| | GCATTGAAATTGTTGCAAACCCCACCTGACAGATTGAGGGCCCAGGCAGGAAGGCGT |
| | TGAGATGGAGGTACAGGAGTCAAGTAACTGAAAGTTTTTATGATAACTAACAACAAA |
| | GGGTCGTTTCTGGCCAGCGAATGACAAGAACAAGATTCCACATTTCCGTGTAGAGGC |
| | TTGCCATCGAATGTGAGCGGGCGGGCCGCGGACCCGACAAAACCCTTACGACGTGGT |
| | AAGAAAAACGTGGCGGGCACTGTCCCTGTAGCCTGAAGACCAGCAGGAGACGATCG |
| | GAAGCATCACAGCACAACCGGTgcagcagcagctcggatagtatcgacacactctggacgctggtcgtgtgatggac |
| | tgttgccgccacacttgctgccttgacctgtgaatatccctgccgcttttatcaaacagcctcagtgtgtttgatcttgtgtgtacgcgcttttgcg |
| | agttgctagctgcttgtgctatttgcgaataccacccccagcatccccttccctcgtttcatatcgcttgcatcccaaccgcaacttatctacgc |
| | tgtcctgctatccctcagcgctgctcctgctcctgctcactgcccctcgcacagccttggtttgggctccgcctgtattctcctggtactgcaacc |
| | 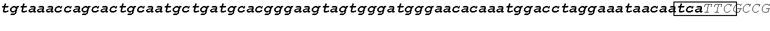 |
| | 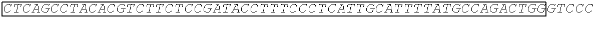 |
| | 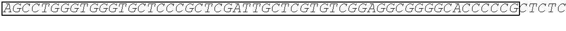 |
| | 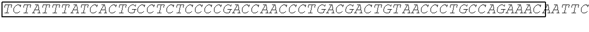 |
| | 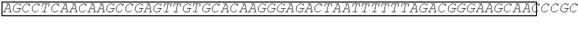 |
| | 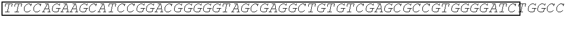 |
| | 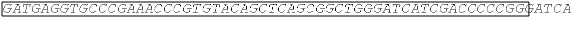 |
| | 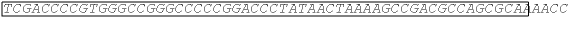 |
| | 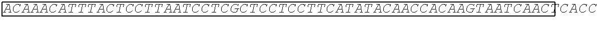 |
| | 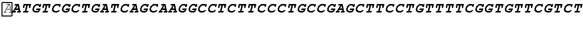 |
| | TCTACTGGTCCCTGCCAATCTTTGCGGTCCCGTACCGTATCCGGTTTGCTAACATGGGG |
| | AAGCGTAACGACATGCTGGACTGGGCTAAGGCGCTGGTGGCCTTTTTCCGCGTCACCG |
| | TCCTGAAGCAGGGCGAGCAGGGCCTGTACCGGGGCGGGACGTGCCTCTACCTGTGCAA |
| | CCACCGAAGCTGGGCCGACTTTTTCATCGACGTCTACCTGACGGAGGGCCGTGCGGCC |
| | CTGATGTCGCGGTGGCTCGTGTTTTTCGTCTTCCCTGTCTTTTGCACGAGCTGCCTCATC |
| | CTCCGAGGCATCGTCCTGTTTAAGCGGGGCACGATCCTGGACAAGGAGGCCTTCAACG |
| | CCTGGCTCGACGCCACGCTCCGACGCTCGATCGTGCCTGGCCTGGTGGTCTACCCCGA |
| | GGGGCATCGCTCCCTCAAGCCCACCTCGCTCCCCCTGAAGCGAGGCATGCTGCACTTC |
| | GCGTTTGGCCGTCAGCTCCCGGTGCAACTGATCGTGACGCGCGGAAAGGAGGACGTGC |
| | TGTCCGAGAAGACCATGGCCATGAAGTACGGCCGCACGCTGGTGACGAGCTACTCTAA |
| | GGTGCTGAAGCCCTCTGACTACGCCAGCTTCGACGCCTTCATGACGGACATCCAGACGT |
| | GCTGGGACTCTTGCTGGCGTGCCGCTTACGAGGCCCCGACCAAGGGCGTCCCCCAGCT |
| | CTCCATGGTCGGCGCCCACGAGTACGACTACCCCCGGACCATTTGGTGGCAGCAACTCT |
| | GGATCACCCTGGCCTCCATCCTGATCTTCGCTGGCGTGCTCTACGGCACCTGGCGCGGC |
| | CTGGCTTCCCTCCTGGCTGCCGCGACCACGAGCGGCGCGCTGGGCCCACAGGTCCTGG |
| | TGACCGTGGCCGCGGCCTGGGCGGGCTCCTCGCTCCTGCGGTCGCTGCTGTGAgtaccacg |
| | cccgcgcggcgcacctgacctgttctctcgagggcgcctgttctgccttgcgaaacaagcccctggagcatgcgtgcatgatcgtctctggcg |
| | ccccgccgcgcggtttgtcgccctcgcgggcgccccggccgcgggggcgcattgaaattgttgcaaaccccacctgacagattgagggcc |
| | caggcaggaaggcgttgagatggaggtacaggagtcaagtaactgaaagtttttatgataactaacaacaaagggtcgtttctggccagcgaa |
| | tgacaagaacaagattccacatttccgtgtagaggcttgccatcgaatgtgagcggggggccgcggacccgacaaaacccttacgacgtg |
| | gtaagaaaaacgtggcgggcactgtccctgtagcctgaagaccagcaggagacgatcggaagcatcacagcacaggccggcccgcgtct |
| | cgaacagagcgcggcggccgcatcgatacctgcaggacagcgccatgccacgccctttgatggcttcaagtacgattacggtgttggat |
| | tgtgtgtttgttgcgtagtgtgcatggtttagaataatacacttgatttcttgctcacggcaatctcggcttgtccgcaggttcaaccccat |
| | ttcggagtctcaggtcagccgcgcaatgaccagccgctacttcaaggacttgcacgacaacgccgaggtgagctatgtttaggacttg |
| | attggaaattgtcgtcgacgcatattcgcgctccgcgacagcacccaagcaaaatgtcaagtgcgttccgatttgcgtccgcaggtcg |
| | atgttgtgatcgtcggcgccggatccgccggtctgtcctgcgcttacgagctgaccaagcaccctgacgtccgggtacgcgagctgag |
| | attcgattagacataaattgaagattaaacccgtagaaaaatttgatggtcgcgaaactgtgctcgattgcaagaaattgatcgtcctc |
| | cactccgcaggtcgccatcatcgagcagggcgttgctcccggcggcggcgcctggctggggggacagctgttctcggccatgtgtgta |
| | cgtagaaggatgaatttcagctggttttcgttgcacagctgtttgtgcatgatttgtttcagactattgttgaatgtttttagatttcttagg |
| | atgcatgatttgtctgcatgcgact |

The construct can be written as 5′Thia4::CrTUB2:ScSUC2:PmPGH:CvNR:PmG3PDH:VitrLPAAT2:PmPGH::3′Thia4. Proceeding in the 5′ to 3′ direction bold, lowercase sequences represent genomic DNA from CHK22 that permit targeted integration at the Thia4 locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR that enables amplification of the VitrLPAAT2 gene, is indicated by uppercase underlined text followed by a linker, indicated by lowercase, bold italics, which contains a C. vulgaris nitrate reductase 3′-UTR. The P. moriformis PmG3PDH promoter, indicated by boxed italicized text, drive the expression of the VitrLPAAT2. The initiator ATG and terminator TGA codons of the VitrLPAAT2 are indicated by uppercase, bold italics, while the remainder of the gene is indicated by bold italics. The PmPGH 3′-UTR is indicated by lowercase underlined text followed by the CHK22 Thia4 genomic region indicated by bold, lowercase text.

Table 77 shows the fatty acid/TAG profiles of seven primary transformants of CHK22 transformed with pCHK1211. The P. moriformis base strain CHK22 is shown as a non-transgenic control. Strains were grown for 120 hrs in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 77
Screen of primary transformants of CHK22 with pCHK1211.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK1211-1 | 0.04 | 1.34 | 27.64 | 2.85 | 58.00 | 7.77 | 0.48 | 0.25 | 0.00 | 4.06 |
| pCHK1211-2 | 0.04 | 1.36 | 28.07 | 2.68 | 57.65 | 7.88 | 0.49 | 0.24 | 0.00 | 4.13 |
| pCHK1211-3 | 0.04 | 1.35 | 27.74 | 2.58 | 57.77 | 8.18 | 0.50 | 0.23 | 0.00 | 4.28 |
| pCHK1211-4 | 0.04 | 1.37 | 27.89 | 2.71 | 57.61 | 8.03 | 0.52 | 0.24 | 0.00 | 3.99 |
| pCHK1211-5 | 0.04 | 1.33 | 27.71 | 2.65 | 58.04 | 7.82 | 0.50 | 0.23 | 0.00 | 4.16 |
| pCHK1211-6 | 0.04 | 1.60 | 31.43 | 2.20 | 54.50 | 7.52 | 0.50 | 0.21 | 0.00 | 4.07 |
| pCHK1211-7 | 0.04 | 1.36 | 27.57 | 2.79 | 58.16 | 7.68 | 0.48 | 0.24 | 0.00 | 3.94 |
| CHK22 | 0.04 | 1.27 | 27.29 | 2.45 | 59.08 | 7.45 | 0.54 | 0.23 | 0.00 | 1.5 |
| Control | | | | | | | | | | |

### Example 37: Expressing Colemanosphaera charkowiensis (CchaLPAAT2) in P. moriformis Strain CHK22 Using the PmG3PDH Promoter

CcIwLPAAT2 was introduced into a P. moriformis strain CHK22. The expression construct pCHK1212 contained 5′ and 3′ homology arms to permit its targeted integration into the genome and is shown in Table 78.

TABLE 78
Integrative sequences for the transformation of P. moriformis with pCHK1212
encoding CchaLPAAT2.

| SEQ ID | |
|---|---|
| NO: | Nucleotide Sequence |
| 67 | ccctcaactgcgacgctgggaaccttctccgggcaggcgatgtgcgtgggtttgcctccttggcacggctctacaccgtcgagtacgcc |
| | atgaggcggtgatggctgtgtcggttgccacttcgtccagagacggcaagtcgtccatcctctgcgtgtgtggcgcgacgctgcagca |
| | gtccctctgcagcagatgagcgtgactttggccatttcacgcactcgagtgtacacaatccatttttcttaaagcaaatgactgctgatt |
| | gaccagatactgtaacgctgatttcgctccagatcgcacagatagcgaccatgttgctgcgtctgaaaatctggattccgaattcgacc |
| | ctggcgctccatccatgcaacagatggcgacacttgttacaattcctgtcacccatcggcatggagcaggtccacttagattcccgatc |
| | acccacgcacatctcgctaatagtcattcgttcgtgtcttcgatcaatctcaagtgagtgtgcatggatcttggttgacgatgcggtatg |
| | ggtttgcgccgctggctgcagggtctgcccaaggcaagctaacccagctcctctccccgacaatactctcgcaggcaaagccggtca |
| | cttgccttccagattgccaataaactcaattatggcctctgtcatgccatccatgggtctgatgaatggtcacgctcgtgtcctgaccgtt |
| | 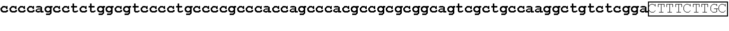 |
| | 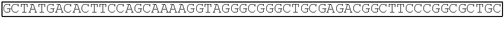 |
| | 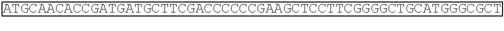 |
| | 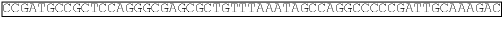 |
| | 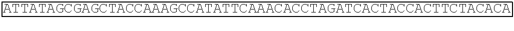 |
| | 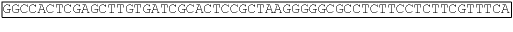 |
| | 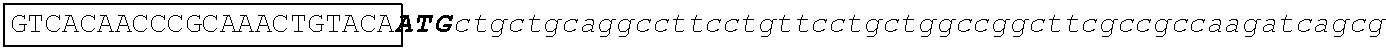 |
| | cctccatgacgaacgagacgtccgaccgccccctggtgcacttcacccccaacaagggctggatgaacgaccccaacggcctgtggta |
| | cgacgagaaggacgccaagtggcacctgtacttccagtacaacccgaacgacaccgtctgggggacgcccttgttctggggccacgcc |
| | acgtccgacgacctgaccaactgggaggaccagcccatcgccatcgccccgaagcgcaacgactccggcgccttctccggctccatgg |
| | tggtggactacaacaacacctccggcttcttcaacgacaccatcgacccgcgccagcgctgcgtggccatctggacctacaacaccccg |
| | gagtccgaggagcagtacatctcctacagcctggacggcggctacaccttcaccgagtaccagaagaaccccgtgctggccgccaact |
| | ccacccagttccgcgacccgaaggtcttctggtacgagccctcccagaagtggatcatgaccgcggccaagtcccaggactacaagat |
| | cgagatctactcctccgacgacctgaagtcctggaagctggagtccgcgttcgccaacgagggcttcctcggctaccagtacgagtgccc |
| | cggcctgatcgaggtccccaccgagcaggaccccagcaagtcctactgggtgatgttcatctccatcaaccccggcgccccggccggc |
| | ggctccttcaaccagtacttcgtcggcagcttcaacggcacccacttcgaggccttcgacaaccagtcccgcgtggtggacttcggcaag |
| | gactactacgccctgcagaccttcttcaacaccgacccgacctacgggagcgccctgggcatcgcgtgggcctccaactgggagtactc |
| | cgccttcgtgcccaccaacccctggcgctcctccatgtccctcgtgcgcaagttctccctcaacaccgagtaccaggccaacccggagac |
| | ggagctgatcaacctgaaggccgagccgatcctgaacatcagcaacgccggcccctggagccggttcgccaccaacaccacgttgac |
| | gaaggccaacagctacaacgtcgacctgtccaacagcaccggcaccctggagttcgagctggtgtacgccgtcaacaccacccagac |
| | gatctccaagtccgtgttcgcggacctctccctctggttcaagggcctggaggaccccgaggagtacctccgcatgggcttcgaggtgtcc |
| | gcgtcctccttcttcctggaccgcgggaacagcaaggtgaagttcgtgaaggagaacccctacttcaccaaccgcatgagcgtgaacaa |
| | ccagcccttcaagagcgagaacgacctgtcctactacaaggtgtacggcttgctggaccagaacatcctggagctgtacttcaacgacg |
| | gcgacgtcgtgtccaccaacacctacttcatgaccaccgg aacgccctgggctccgtgaacatgacgacgggggtggacaacctgttc |
| | tacatcgacaagttccaggtgcgcgaggtcaagtgactTAAGACGCCCGCGCGGCGCACCTGACCTGTTCT |
| | CTCGAGGGCGCCTGTTCTGCCTTGCGAAACAAGCCCCTGGAGCATGCGTGCATGATCG |
| | TCTCTGGCGCCCCGCCGCGCGGTTTGTCGCCCTCGCGGGCGCCGCGGCCGCGGGGGC |
| | GCATTGAAATTGTTGCAAACCCCACCTGACAGATTGAGGGCCCAGGCAGGAAGGCGT |
| | TGAGATGGAGGTACAGGAGTCAAGTAACTGAAAGTTTTTATGATAACTAACAACAAA |
| | GGGTCGTTTCTGGCCAGCGAATGACAAGAACAAGATTCCACATTTCCGTGTAGAGGC |
| | TTGCCATCGAATGTGAGCGGGCGGGCCGCGGACCCGACAAAACCCTTACGACGTGGT |
| | AAGAAAAACGTGGCGGGCACTGTCCCTGTAGCCTGAAGACCAGCAGGAGACGATCG |
| | GAAGCATCACAGCACAACCGGTgcagcagcagctcggatagtatcgacacactctggacgctggtcgtgtgatggac |
| | tgttgccgccacacttgctgccttgacctgtgaatatccctgccgcttttatcaaacagcctcagtgtgtttgatcttgtgtgtacgcgcttttgcg |
| | agttgctagctgcttgtgctatttgcgaataccacccccagcatccccttccctcgtttcatatcgcttgcatcccaaccgcaacttatctacgc |
| | tgtcctgctatccctcagcgctgctcctgctcctgctcactgcccctcgcacagccttggtttgggctccgcctgtattctcctggtactgcaacc |
| | 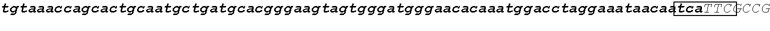 |
| | 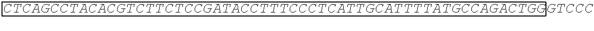 |
| | 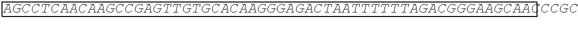 |
| | 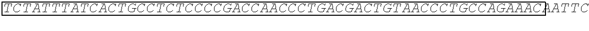 |
| | 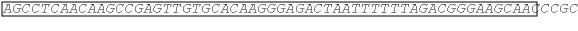 |
| | 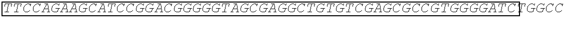 |
| | 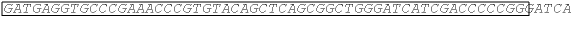 |
| | 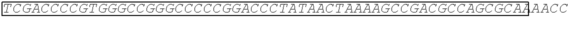 |
| | 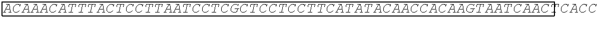 |
| | 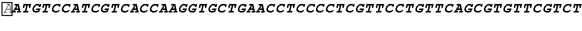 |
| | TTTACTGGTCCCTGCCCCTGTTTGCGATCCTGTACCGCATCCGCTTCGCCAACGTGGGC |
| | AAGCGCAACGACATGCTGGACTGGGCGAAGACGCTCGTCGGCTTTTTCCACGTGACCA |
| | TCCTGAAGCAGGGCGAGCTGTCTCTCTACCGGGACGGGCCGTGCCTGTACCTGTGCAA |
| | CCACCGCTCCTGGGCCGACTTCTTTATCGACACCTACCTGACGGAGGGTCGCGCTGCCC |
| | TGATGAGCCGCTGGCTGGTGTACTTCGTGTTCCCGGTCTTCTGCACGTCCGTGATTATC |
| | CTGCGCGGCATCGTGCTGTTCAAGCGCGGGTCTATCGCGGACAAGGAGGCCTTCAACG |
| | CGTGGCTGGACGCCACGCTGCGTAAGTCCATTGTCCCCGCGCTCCTGGTGTACCCGGA |
| | GGGCCACCGCTCGCTGCGCCCCACGTCTCTCCCTCTGAAGCGCGGCATGCTGCACTAC |
| | GCCTACAGCCGCCGACTCCCTGTGCAGCTCATCGTCACGTCGGGCAAGGAGGCCGTCA |
| | TGAGCGAGAAGACCATGAGCGTCCGCTTCGGCCGCACCCTCGTCACCACGTACTCCAA |
| | GGTGCTGCGCCCCGCGGACTACCCCACGTTTGACGCCTTCTTTTCCGACATCCAAGCTA |
| | CCTGGGACGGCTGCTGGGACGCCGCTTACAAGCAGCCGTCTGCGGGTGCGCCCCAGCT |
| | CCGCCTGGTGGACCCGCATGGCTACAACTACCCGGCCCGCATGTGGTGGCTCCAACTG |
| | GGCGTGACCCTCCTGTCGATCGGGGTGATGGCCGCTGTGATCGCCGCGAGCTGGTGGG |
| | GTCTGGGCGCTCTCCTGGCCGCGACGGGCCCGGCTAAGCAGGTGGTCCTGGGCGTCGT |
| | GGTCTCGCTGCTCGGCGCGAGCCTCCTGCGATCGTGCCTGTGAgtaccacgcccgcgcggcgcacc |
| | tgacctgttctctcgagggcgcctgttctgccttgcgaaacaagcccctggagcatgcgtgcatgatcgtctctggcgccccgccgcgcggttt |
| | gtcgccctcgcgggcgccccggccgcgggggcgcattgaaattgttgcaaaccccacctgacagattgagggcccaggcaggaaggcgtt |
| | gagatggaggtacaggagtcaagtaactgaaagtttttatgataactaacaacaaagggtcgtttctggccagcgaatgacaagaacaagattc |
| | cacatttccgtgtagaggcttgccatcgaatgtgagcggggggccgcggacccgacaaaacccttacgacgtggtaagaaaaacgtggcg |
| | ggcactgtccctgtagcctgaagaccagcaggagacgatcggaagcatcacagcacaggccggcccgcgtctcgaacagagcgcggcg |
| | gccgcatcgatacctgcaggacagcgccatgccacgccctttgatggcttcaagtacgattacggtgttggattgtgtgtttgttgcgtag |
| | tgtgcatggtttagaataatacacttgatttcttgctcacggcaatctcggcttgtccgcaggttcaaccccatttcggagtctcaggtca |
| | gccgcgcaatgaccagccgctacttcaaggacttgcacgacaacgccgaggtgagctatgtttaggacttgattggaaattgtcgtcg |
| | acgcatattcgcgctccgcgacagcacccaagcaaaatgtcaagtgcgttccgatttgcgtccgcaggtcgatgttgtgatcgtcggc |
| | gccggatccgccggtctgtcctgcgcttacgagctgaccaagcaccctgacgtccgggtacgcgagctgagattcgattagacataa |
| | attgaagattaaacccgtagaaaaatttgatggtcgcgaaactgtgctcgattgcaagaaattgatcgtcctccactccgcaggtcgc |
| | catcatcgagcagggcgttgctcccggcggcggcgcctggctggggggacagctgttctcggccatgtgtgtacgtagaaggatgaa |
| | tttcagctggttttcgttgcacagctgtttgtgcatgatttgtttcagactattgttgaatgtttttagatttcttaggatgcatgatttgtctg |
| | catgcgact |

The construct can be written as 5′Thia4::CrTUB2:ScSUC2:PmPGH:CvNR:PmG3PDH:CchaLPAAT2:PmPGH::3′Thia4. Proceeding in the 5′ to 3′ direction, bold, lowercase sequences represent genomic DNA from CHK22 that permit targeted integration at the Thia4 locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR that enables amplification of the CchaLPAAT2 gene, is indicated by uppercase underlined text followed by a linker, indicated by lowercase, bold italics, which contains a C. vulgaris nitrate reductase 3′-UTR. The P. moriformis PmG3PDH promoter, indicated by boxed italicized text, drive the expression of the CchaLPAAT2. The initiator ATG and terminator TGA codons of the CchaLPAAT2 are indicated by uppercase, bold italics, while the remainder of the gene is indicated by bold italics. The PmPGH 3′-UTR is indicated by lowercase underlined text followed by the CHK22 Thia4 genomic region indicated by bold, lowercase text.

Table 79 shows the fatty acid/TAG profiles of eight primary transformants of CHK22 transformed with pCHK1212. The P. moriformis base strain CHK22 is shown as a non-transgenic control. Strains were grown for 96 hrs in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 79
Screen of primary transformants of CHK22 with pCHK1212.

| | | | | | | | | | | TAG Profile |
|---|---|---|---|---|---|---|---|---|---|---|
| | FAME Profiles | | | | | | | | | m/z 577/603 |
| Sample | C12:0 | C14:0 | C16:0 | C18:0 | C18:1 | C18:2 | C18:3 alpha | C20:0 | C20:1 | (OP:OO) |
| pCHK1212-1 | 0.04 | 1.32 | 27.29 | 2.88 | 58.69 | 7.56 | 0.54 | 0.24 | 0.03 | 2.4 |
| pCHK1212-2 | 0.03 | 1.24 | 27.36 | 2.92 | 59.47 | 6.87 | 0.50 | 0.23 | 0.00 | 2.2 |
| pCHK1212-3 | 0.03 | 1.22 | 27.50 | 2.91 | 59.53 | 6.70 | 0.49 | 0.23 | 0.00 | 1.8 |
| pCHK1212-4 | 0.03 | 1.25 | 27.81 | 2.75 | 58.58 | 7.43 | 0.49 | 0.21 | 0.03 | 2.5 |
| pCHK1212-5 | 0.03 | 1.33 | 27.56 | 2.81 | 58.47 | 7.62 | 0.51 | 0.22 | 0.00 | 2.6 |
| pCHK1212-6 | 0.03 | 1.30 | 27.79 | 2.79 | 58.61 | 7.34 | 0.51 | 0.22 | 0.00 | 2.2 |
| pCHK1212-7 | 0.03 | 1.27 | 27.96 | 2.68 | 58.71 | 7.18 | 0.51 | 0.21 | 0.00 | 2.3 |
| pCHK1212-8 | 0.04 | 1.34 | 27.76 | 2.94 | 58.84 | 7.00 | 0.50 | 0.22 | 0.00 | 2.1 |
| CHK22 | 0.04 | 1.4 | 25.8 | 2.69 | 61.83 | 6.51 | 0.36 | 0.16 | 0 | 1.5 |
| Control | | | | | | | | | | |

### Example 38: Expressing Pleodorina Japonica (PjapLPAAT2) in P. moriformis Strain CHK22 Using the PmG3PDH Promoter

PjapLPAAT2 was introduced into a P. moriformis strain CHK22. The expression construct pCHK1213 contained 5′ and 3′ homology arms to permit its targeted integration into the genome and is shown in Table 80.

TABLE 80
Integrative sequences for the transformation of P. moriformis with pCHK1213 encoding PjapLPAAT2.

| SEQ | |
|---|---|
| ID | |
| NO: | Nucleotide Sequence |
| 68 | ccctcaactgcgacgctgggaaccttctccgggcaggcgatgtgcgtgggtttgcctccttggcacggctctacaccgtcgagtacgcc |
| | atgaggcggtgatggctgtgtcggttgccacttcgtccagagacggcaagtcgtccatcctctgcgtgtgtggcgcgacgctgcagca |
| | gtccctctgcagcagatgagcgtgactttggccatttcacgcactegagtgtacacaatccatttttcttaaagcaaatgactgctgatt |
| | gaccagatactgtaacgctgatttcgctccagatcgcacagatagcgaccatgttgctgcgtctgaaaatctggattccgaattegacc |
| | ctggcgctccatccatgcaacagatggcgacacttgttacaattcctgtcacccateggcatggagcaggtccacttagattcccgatc |
| | acccacgcacatctcgctaatagtcattcgttcgtgtcttcgatcaatctcaagtgagtgtgcatggatcttggttgacgatgcggtatg |
| | ggtttgcgccgctggctgcagggtctgcccaaggcaagctaacccagctcctctccccgacaatactctcgcaggcaaagccggtca |
| | cttgccttccagattgccaataaactcaattatggcctctgtcatgccatccatgggtctgatgaatggtcacgctcgtgtcctgaccgtt |
| | 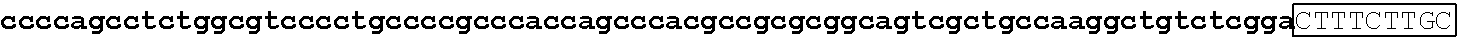 |
| | 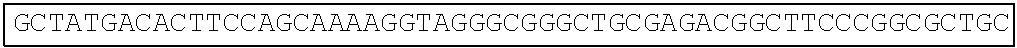 |
| | 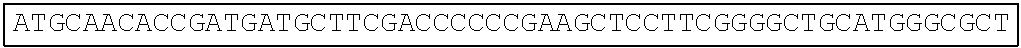 |
| | 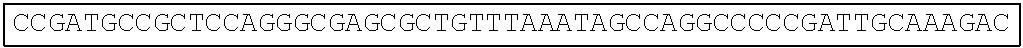 |
| | 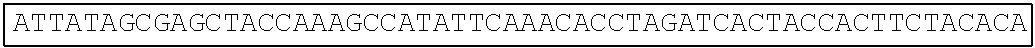 |
| | 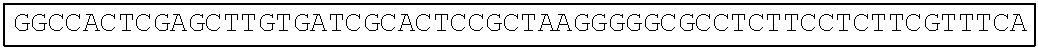 |
| | 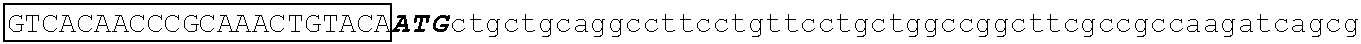 |
| | cctccatgacgaacgagacgtccgaccgccccctggtgcacttcacccccaacaagggctggatgaacgaccccaacggcctgtggta |
| | cgacgagaaggacgccaagtggcacctgtacttccagtacaacccgaacgacaccgtctgggggacgcccttgttctggggccacgcc |
| | acgtccgacgacctgaccaactgggaggaccagcccatcgccatcgccccgaagcgcaacgactccggcgccttctccggctccatgg |
| | tggtggactacaacaacacctccggcttcttcaacgacaccatcgacccgcgccagcgctgcgtggccatctggacctacaacaccccg |
| | gagtccgaggagcagtacatctcctacagcctggacggcggctacaccttcaccgagtaccagaagaaccccgtgctggccgccaact |
| | ccacccagttccgcgacccgaaggtcttctggtacgagccctcccagaagtggatcatgaccgcggccaagtcccaggactacaagat |
| | cgagatctactcctccgacgacctgaagtcctggaagctggagtccgcgttcgccaacgagggcttcctcggctaccagtacgagtgccc |
| | cggcctgatcgaggtccccaccgagcaggaccccagcaagtcctactgggtgatgttcatctccatcaaccccggcgccccggccggc |
| | ggctccttcaaccagtacttcgtcggcagcttcaacggcacccacttcgaggccttcgacaaccagtcccgcgtggtggacttcggcaag |
| | gactactacgccctgcagaccttcttcaacaccgacccgacctacgggagcgccctgggcatcgcgtgggcctccaactgggagtactc |
| | cgccttcgtgcccaccaacccctggcgctcctccatgtccctcgtgcgcaagttctccctcaacaccgagtaccaggccaacccggagac |
| | ggagctgatcaacctgaaggccgagccgatcctgaacatcagcaacgccggcccctggagccggttcgccaccaacaccacgttgac |
| | gaaggccaacagctacaacgtcgacctgtccaacagcaccggcaccctggagttcgagctggtgtacgccgtcaacaccacccagac |
| | gatctccaagtccgtgttcgcggacctctccctctggttcaagggcctggaggaccccgaggagtacctccgcatgggcttcgaggtgtcc |
| | gcgtcctccttcttcctggaccgcgggaacagcaaggtgaagttcgtgaaggagaacccctacttcaccaaccgcatgagcgtgaacaa |
| | ccagcccttcaagagcgagaacgacctgtcctactacaaggtgtacggcttgctggaccagaacatcctggagctgtacttcaacgacg |
| | gcgacgtcgtgtccaccaacacctacttcatgaccaccgggaacgccctgggctccgtgaacatgacgacgggggtggacaacctgttc |
| | tacatcgacaagttccaggtgcgcgaggtcaagtgactTAAGACGCCCGCGCGGCGCACCTGACCTGTTCT |
| | CTCGAGGGCGCCTGTTCTGCCTTGCGAAACAAGCCCCTGGAGCATGCGTGCATGATCG |
| | TCTCTGGCGCCCCGCCGCGCGGTTTGTCGCCCTCGCGGGCGCCGCGGCCGCGGGGGC |
| | GCATTGAAATTGTTGCAAACCCCACCTGACAGATTGAGGGCCCAGGCAGGAAGGCGT |
| | TGAGATGGAGGTACAGGAGTCAAGTAACTGAAAGTTTTTATGATAACTAACAACAAA |
| | GGGTCGTTTCTGGCCAGCGAATGACAAGAACAAGATTCCACATTTCCGTGTAGAGGC |
| | TTGCCATCGAATGTGAGCGGGCGGGCCGCGGACCCGACAAAACCCTTACGACGTGGT |
| | AAGAAAAACGTGGCGGGCACTGTCCCTGTAGCCTGAAGACCAGCAGGAGACGATCG |
| | GAAGCATCACAGCACAACCGGTgcagcagcagctcggatagtatcgacacactctggacgctggtcgtgtgatggac |
| | tgttgccgccacacttgctgccttgacctgtgaatatccctgccgcttttatcaaacagcctcagtgtgtttgatcttgtgtgtacgcgcttttgc |
| | gagttgctagctgcttgtgctatttgcgaataccacccccagcatccccttccctegtttcatategcttgcatcccaaccgcaacttatctacgc |
| | tgtcctgctatccctcagcgctgctcctgctcctgctcactgcccctcgcacagccttggtttgggctccgcctgtattctcctggtactgcaacc |
| | 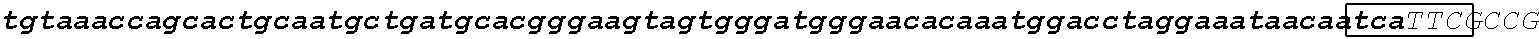 |
| | 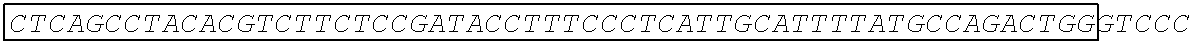 |
| | 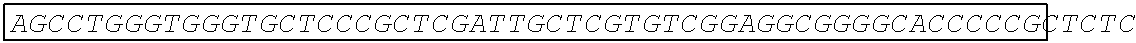 |
| | 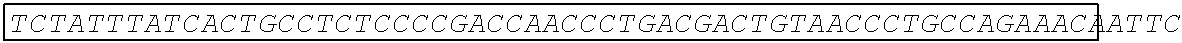 |
| | 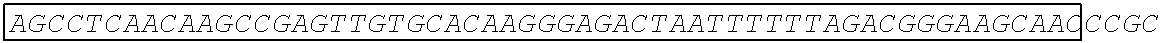 |
| | 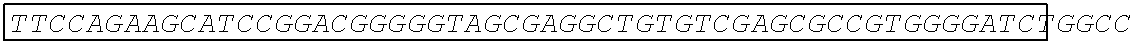 |
| | 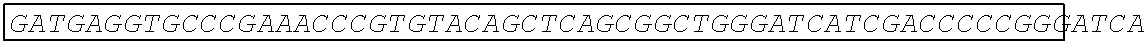 |
| | 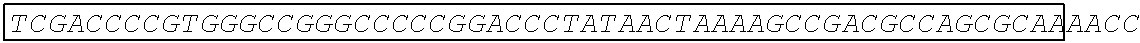 |
| | 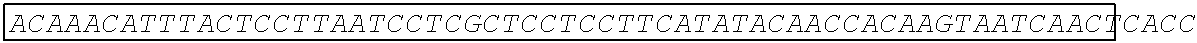 |
| | 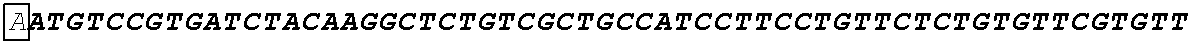 |
| | CTACTGGTCTCTGCCCCTGTTCGCGTCTGTCTACCGCATCCGGTTTGCGAACTTCGGCA |
| | AGCGCAACGACATGCTGGACTGGGCGAAGGCCCTCGTGGCCTTCTTTCGCGTCACCGT |
| | GCTCAAGCAAGGCGAGATGTCGCTCTACCGCGGAGGCCCTTGTCTCTACCTGTGCAACC |
| | ATCGGTCGTGGGCTGACTTCTTTATCGACACGTACCTGACCGAGGGCCGCGCGGCCCT |
| | GATGAGCCGCTGGCTGGTCTACTTCGTGTTTCCGGTGTTTTGTACCAGCGTGCTGATCC |
| | TGCGGGGCATCGTCCTGTTTAAGCGGGGTCACATCGTGGACAAGGAGGCGTTCAACGC |
| | GTGGCTCGACGCCACCCTGTTCAAGTCTACGGTGCCGGGCCTCCTGGTGTACCCCGAG |
| | GGCCACCGCTCCCTCAAGCCCGCCTCCCTGCCCCTGAAGCGTGGCATGCTCCACTTTGC |
| | TTTTTCCCGCAAGCTGCCCGTCCAGGTCATTTGCACCCGCGGCAAGGAGGAGGTCCTGT |
| | GCGAGAAGACGCTGTCCGCGCGGTGGGGCCGCACGCTGGTGGTCACCTTCTCCAAGGT |
| | GATCAAGCCGTCCGAGTACGACTCGTTCGACGCGTTTTTCAACGACGTGCAATCTACCT |
| | GGGACGCGTGCTGGCAGCTCTCCTACCACCAGCCAACGGCGGGCGTCCCGGAGCTGTC |
| | CATGAAGGACGCCCACGAGTACGACTACCCCCGCTCTATGTGGTGGCTGCAACTGGGT |
| | ATCACCCTCCTGTCCTTTATTATCCTGGCCGCGGTGCTGTGGGGCTCTTGGGTCGCTGT |
| | GCGCGCGGGTTTCGCCACGCTCGGCAGCGTCCAACAGGCCGTGGTCCTGGGCACCGCG |
| | GGGTGGATCGGCGCCTCGATGGTGTGCGGCTTCCTCTGAgtaccacgcccgogoggcgcacctgacct |
| | gttctctcgagggcgcctgttctgccttgcgaaacaagcccctggagcatgcgtgcatgatcgtctctggcgccccgccgcgcggtttgtcgc |
| | cctcgcgggcgccccggccgcgggggcgcattgaaattgttgcaaaccccacctgacagattgagggcccaggcaggaaggcgttgagat |
| | ggaggtacaggagtcaagtaactgaaagtttttatgataactaacaacaaagggtcgtttctggccagcgaatgacaagaacaagattccacat |
| | ttccgtgtagaggcttgccatcgaatgtgagcggggggccgcggacccgacaaaacccttacgacgtggtaagaaaaacgtgggggca |
| | ctgtccctgtagcctgaagaccagcaggagacgatcggaagcatcacagcacaggccggcccgcgtctcgaacagagcgcggcggccg |
| | catcgatacctgcaggacagcgccatgccacgccctttgatggcttcaagtacgattacggtgttggattgtgtgtttgttgcgtagtgtg |
| | catggtttagaataatacacttgatttcttgctcacggcaatctcggcttgtccgcaggttcaaccccattteggagtctcaggtcagccg |
| | cgcaatgaccagccgctacttcaaggacttgcacgacaacgccgaggtgagctatgtttaggacttgattggaaattgtcgtcgacgc |
| | atattegcgctccgcgacagcacccaagcaaaatgtcaagtgegttccgatttgcgtccgcaggtcgatgttgtgategteggcgccg |
| | gatccgccggtctgtcctgcgcttacgagctgaccaagcaccctgacgtccgggtacgcgagctgagattcgattagacataaattga |
| | agattaaacccgtagaaaaatttgatggtcgcgaaactgtgctcgattgcaagaaattgatcgtcctccactccgcaggtegccatca |
| | tcgagcagggcgttgctcccggcggcggcgcctggctggggggacagctgttctcggccatgtgtgtacgtagaaggatgaatttca |
| | gctggttttcgttgcacagctgtttgtgcatgatttgtttcagactattgttgaatgtttttagatttcttaggatgcatgatttgtctgcatgc |
| | gact |

The construct can be written as 5′ Thia4::CrTUB2:ScSUC2:PmPGH:CvNR:PmG3PDH:PjapLPAAT2:PmPGH::3′ Thia4. Proceeding in the 5′ to 3′ direction, bold, lowercase sequences represent genomic DNA from CHK22 that permit targeted integration at the Thia4 locus via homologous recombination, the C. reinhwdiii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR that enables amplification of the PjapLPAAT2 gene, is indicated by uppercase underlined text followed by a linker, indicated by lowercase, bold italics, which contains a C. vulgaris nitrate reductase 3′-UTR. A PmG3PDH promoter from P. moriformis, indicated by boxed italicized text, drive the expression of the PjapLPAAT2. The initiator ATG and terminator TGA codons of the PjapLPAAT2 are indicated by uppercase, bold italics, while the remainder of the gene is indicated by bold italics. The PmPGH 3′-UTR is indicated by lowercase underlined text followed by the CHK22 Thia4 genomic region indicated by bold, lowercase text.

Table 81 shows the fatty acid/TAG profiles of seven primary transformants of CHK22 transformed with pCHK1213. The P. moriformis base strain CHK22 is shown as a non-transgenic control. Strains were grown for 120 hrs in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 81
Screen of primary transformants of CHK22 with pCHK1213.

| | FAME Profiles | | | | | | | | | TAG Profile |
|---|---|---|---|---|---|---|---|---|---|---|
| | | | | | | | C18:3 | | | m/z 577/603 |
| Sample Name | C12:0 | C14:0 | C16:0 | C18:0 | C18:1 | C18:2 | alpha | C20:0 | C20:1 | (OP:OO) |
| pCHK1213-1 | 0.04 | 1.32 | 28.01 | 2.75 | 58.23 | 7.17 | 0.50 | 0.25 | 0.00 | 1.87 |
| pCHK1213-2 | 0.04 | 1.28 | 27.71 | 2.78 | 58.77 | 6.96 | 0.49 | 0.25 | 0.00 | 1.89 |
| pCHK1213-3 | 0.04 | 1.35 | 28.46 | 2.96 | 57.69 | 7.00 | 0.49 | 0.27 | 0.00 | 1.55 |
| pCHK1213-4 | 0.04 | 1.44 | 28.22 | 2.84 | 57.69 | 7.37 | 0.47 | 0.26 | 0.00 | 2.04 |
| pCHK1213-5 | 0.04 | 1.50 | 27.69 | 2.33 | 59.75 | 6.46 | 0.32 | 0.18 | 0.00 | 1.85 |
| pCHK1213-6 | 0.03 | 1.30 | 28.21 | 2.75 | 58.45 | 6.92 | 0.48 | 0.25 | 0.00 | 1.90 |
| pCHK1213-7 | 0.06 | 1.67 | 19.16 | 4.74 | 61.76 | 9.66 | 0.88 | 0.52 | 0.00 | 1.50 |
| CHK22 Control | 0.04 | 1.27 | 27.29 | 2.45 | 59.08 | 7.45 | 0.54 | 0.23 | 0.00 | 1.5 |

### Example 39: Expressing Volvulina boldii (VbolLPAAT2) in P. moriformis Strain CHK22 Using the PmG3PDH Promoter

VbolLPAAT2 was introduced into a P. moriformis strain CHK22. The expression construct pCHK1214 contained 5′ and 3′ homology arms to permit its targeted integration into the genome and is shown in Table 82.

TABLE 82
Integrative sequences for the transformation of P. moriformis with pCHK1214
encoding VbolLPAAT2.

| SEQ | |
|---|---|
| ID | |
| NO: | Nucleotide Sequence |
| 69 | ccctcaactgcgacgctgggaaccttctccgggcaggcgatgtgcgtgggtttgcctccttggcacggctctacaccgtegagtacgcc |
| | atgaggcggtgatggctgtgtcggttgccacttcgtccagagacggcaagtcgtccatcctctgcgtgtgtggcgcgacgctgcagca |
| | gtccctctgcagcagatgagcgtgactttggccatttcacgcactegagtgtacacaatccatttttcttaaagcaaatgactgctgatt |
| | gaccagatactgtaacgctgatttegctccagategcacagatagegaccatgttgctgcgtctgaaaatctggattcegaattegacc |
| | ctggcgctccatccatgcaacagatggcgacacttgttacaattcctgtcacccateggcatggagcaggtccacttagattcccgatc |
| | acccacgcacatctcgctaatagtcattcgttcgtgtcttcgatcaatctcaagtgagtgtgcatggatcttggttgacgatgcggtatg |
| | ggtttgcgccgctggctgcagggtctgcccaaggcaagctaacccagetcctctccccgacaatactctcgcaggcaaagccggtca |
| | cttgccttccagattgccaataaactcaattatggcctctgtcatgccatccatgggtctgatgaatggtcacgctcgtgtcctgaccgtt |
| | 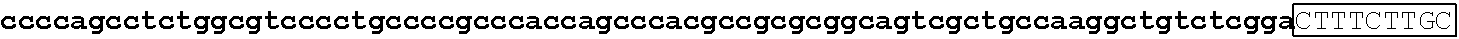 |
| | 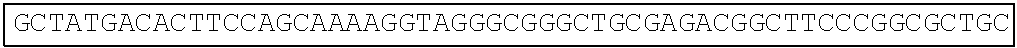 |
| | 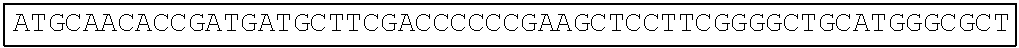 |
| | 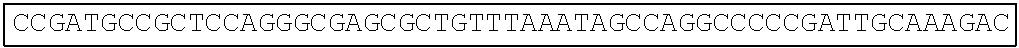 |
| | 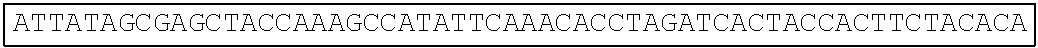 |
| | 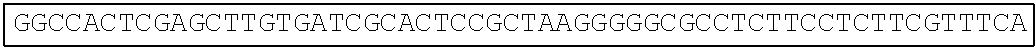 |
| | 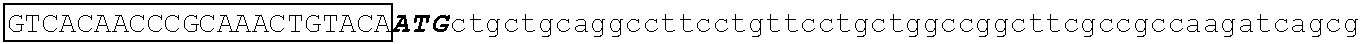 |
| | cctccatgacgaacgagacgtccgaccgccccctggtgcacttcacccccaacaagggctggatgaacgaccccaacggcctgtggta |
| | cgacgagaaggacgccaagtggcacctgtacttccagtacaacccgaacgacaccgtctgggggacgcccttgttctggggccacgcc |
| | acgtccgacgacctgaccaactgggaggaccagcccatcgccatcgccccgaagcgcaacgactccggcgccttctccggctccatgg |
| | tggtggactacaacaacacctccggcttcttcaacgacaccatcgacccgcgccagcgctgcgtggccatctggacctacaacaccccg |
| | gagtccgaggagcagtacatctcctacagcctggacggcggctacaccttcaccgagtaccagaagaaccccgtgctggccgccaact |
| | ccacccagttccgcgacccgaaggtcttctggtacgagccctcccagaagtggatcatgaccgcggccaagtcccaggactacaagat |
| | cgagatctactcctccgacgacctgaagtcctggaagctggagtccgcgttcgccaacgagggcttcctcggctaccagtacgagtgccc |
| | cggcctgatcgaggtccccaccgagcaggaccccagcaagtcctactgggtgatgttcatctccatcaaccccggcgccccggccggc |
| | ggctccttcaaccagtacttcgtcggcagcttcaacggcacccacttcgaggccttcgacaaccagtcccgcgtggtggacttcggcaag |
| | gactactacgccctgcagaccttcttcaacaccgacccgacctacgggagcgccctgggcatcgcgtgggcctccaactgggagtactc |
| | cgccttcgtgcccaccaacccctggcgctcctccatgtccctcgtgcgcaagttctccctcaacaccgagtaccaggccaacccggagac |
| | ggagctgatcaacctgaaggccgagccgatcctgaacatcagcaacgccggcccctggagccggttcgccaccaacaccacgttgac |
| | gaaggccaacagctacaacgtcgacctgtccaacagcaccggcaccctggagttcgagctggtgtacgccgtcaacaccacccagac |
| | gatctccaagtccgtgttcgcggacctctccctctggttcaagggcctggaggaccccgaggagtacctccgcatgggcttcgaggtgtcc |
| | gcgtcctccttcttcctggaccgcgggaacagcaaggtgaagttcgtgaaggagaacccctacttcaccaaccgcatgagcgtgaacaa |
| | ccagcccttcaagagcgagaacgacctgtcctactacaaggtgtacggcttgctggaccagaacatcctggagctgtacttcaacgacg |
| | gcgacgtcgtgtccaccaacacctacttcatgaccaccgggaacgccctgggctccgtgaacatgacgacgggggtggacaacctgttc |
| | tacatcgacaagttccaggtgcgcgaggtcaagtgactTAAGACGCCCGCGCGGCGCACCTGACCTGTTCT |
| | CTCGAGGGCGCCTGTTCTGCCTTGCGAAACAAGCCCCTGGAGCATGCGTGCATGATCG |
| | TCTCTGGCGCCCCGCCGCGCGGTTTGTCGCCCTCGCGGGCGCCGCGGCCGCGGGGGC |
| | GCATTGAAATTGTTGCAAACCCCACCTGACAGATTGAGGGCCCAGGCAGGAAGGCGT |
| | TGAGATGGAGGTACAGGAGTCAAGTAACTGAAAGTTTTTATGATAACTAACAACAAA |
| | GGGTCGTTTCTGGCCAGCGAATGACAAGAACAAGATTCCACATTTCCGTGTAGAGGC |
| | TTGCCATCGAATGTGAGCGGGCGGGCCGCGGACCCGACAAAACCCTTACGACGTGGT |
| | AAGAAAAACGTGGCGGGCACTGTCCCTGTAGCCTGAAGACCAGCAGGAGACGATCG |
| | GAAGCATCACAGCACAACCGGTgcagcagcagctcggatagtatcgacacactctggacgctggtcgtgtgatggac |
| | tgttgccgccacacttgctgccttgacctgtgaatatccctgccgcttttatcaaacagcctcagtgtgtttgatcttgtgtgtacgcgcttttgc |
| | gagttgctagctgcttgtgctatttgcgaataccacccccagcatccccttccctcgtttcatategcttgcatcccaaccgcaacttatctacgc |
| | tgtcctgctatccctcagegctgctcctgctcctgctcactgcccctcgcacagccttggtttgggctccgcctgtattctcctggtactgcaacc |
| | 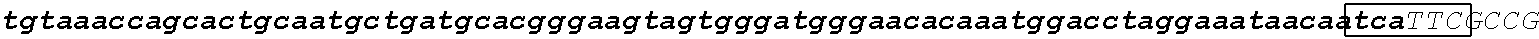 |
| | 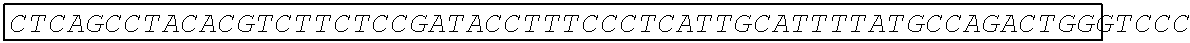 |
| | 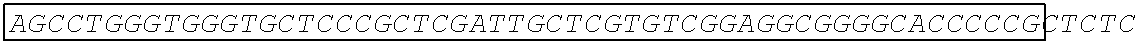 |
| | 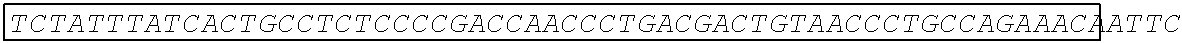 |
| | 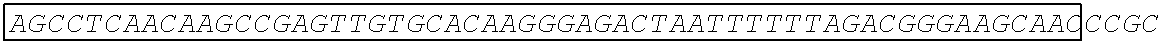 |
| | 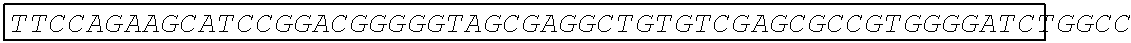 |
| | 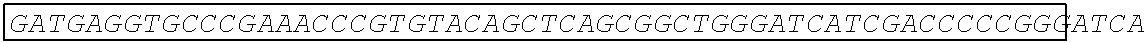 |
| | 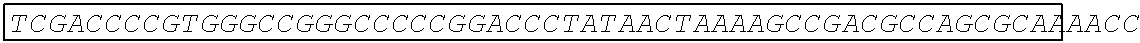 |
| | 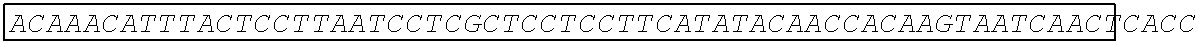 |
| | 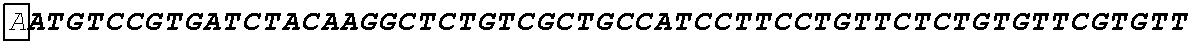 |
| | TCTACTGGAGCCTGCCAATCTTCGCCTCGGTGTACCGCATCCGCTTCGCGCGCGCTGGA |
| | AAGCGCAACGACATGCTGGACTGGGCTACCTCCCTGGTGGCGTTCTTTCGCGTGACCGT |
| | GCTCAAGCAGGGTCAGTTTTCGATCTACCGCGGAGGCCCCTGCATCTACCTGTGCAACC |
| | ACCGTAGCTGGGCCGATTTCTTTATCGACATGTACCTCACGGAGGGCCGCGCCGCGTTC |
| | ATGTCCCGTTGGATGGTCTTCTTTGTCTTCCCCGTCTTCTGCTCCGCCGTGCTGATTCTG |
| | AAGGGCATTATCCTGTTTAAGCGGGGCCGCATCCTGGACAAGGAGGCGTTTAACGCCT |
| | GGCTGGACGCCACCCTGGCGAAGTCGACGATCCCCGGCCTGGTCGTGTACCCGGAGGG |
| | CCATCGCTCTCTGAAGCCGACGTCGCTGCCCCTGAAGCGCGGGATGCTGCACTACGCG |
| | TTTTCCCGTAAGCTCCCAGTGCAACTGATCGTCACCCGCGGCAAGGAGGAGGTGCTGTC |
| | GGAGAAGACCCTGTCCATGCGCTTCGGGCGGACCCTGGTGACCTCTTACTCGAAGGTG |
| | ATGAAGCCCAGCGACTACGGCTCCTTCGATGCCTTTATGACCGACGTGCAGTCTGGCTG |
| | GGACCAGTGCTGGAAGGAGGCCTACTCCGCGCCGACCCACGGCCTGCCGCAGCTCTCG |
| | ATGGCTGGCGCCACCGAGTACGACTACCCCGCTTCCATGCGCTGGCAGCACCTGGCGA |
| | TGACGCTGTTTTCGATCGTCGTGTTTGCGGGTGTCCTGGCCGCGAGCTGGTGGGGCGT |
| | CGGCGCCCTCCTGTCTGCTGCGGGCCCTACGGGCAAGCAGGTGGTCGTGCTCCTGGCC |
| | TCGTGCTGGTTCGGCGCCAGCCTCCTGCGTAGCTTCCTGTGAgtaccacgcccgcgcggcgcacctg |
| | acctgttctctcgagggcgcctgttctgccttgcgaaacaagcccctggagcatgcgtgcatgatcgtctctggcgccccgccgcgcggtttgt |
| | cgccctcgcgggcgccccggccgcgggggcgcattgaaattgttgcaaaccccacctgacagattgagggcccaggcaggaaggcgttg |
| | agatggaggtacaggagtcaagtaactgaaagtttttatgataactaacaaacaaagggtcgtttctggccagcgaatgacaagaacaagattcc |
| | acatttccgtgtagaggcttgccatcgaatgtgagcggggggccgcggacccgacaaaacccttacgacgtggtaagaaaaacgtggcgg |
| | gcactgtccctgtagcctgaagaccagcaggagacgatcggaagcatcacagcacaggccggcccgcgtctcgaacagagcgcggcgg |
| | ccgcatcgatacctgcaggacagcgccatgccacgccctttgatggcttcaagtacgattacggtgttggattgtgtgtttgttgcgtagt |
| | gtgcatggtttagaataatacacttgatttcttgctcacggcaatctcggcttgtccgcaggttcaaccccatttcggagtctcaggtcag |
| | ccgcgcaatgaccagccgctacttcaaggacttgcacgacaacgccgaggtgagctatgtttaggacttgattggaaattgtcgtcga |
| | cgcatattcgcgctccgcgacagcacccaagcaaaatgtcaagtgcgttccgatttgcgtccgcaggtcgatgttgtgatcgtcggcg |
| | ccggatccgccggtctgtcctgcgcttacgagctgaccaagcaccctgacgtccgggtacgcgagctgagattcgattagacataaat |
| | tgaagattaaacccgtagaaaaatttgatggtcgcgaaactgtgctcgattgcaagaaattgatcgtcctccactccgcaggtegcca |
| | tcatcgagcagggcgttgctcccggcggcggcgcctggctggggggacagctgttctcggccatgtgtgtacgtagaaggatgaattt |
| | cagctggttttcgttgcacagctgtttgtgcatgatttgtttcagactattgttgaatgtttttagatttcttaggatgcatgatttgtctgcat |
| | gcgact |

The construct can be written as 5′Thia4::CrTUB2:ScSUC2:PmPGH:CvNR:PmG3PDH:VbolLPAAT2:PmPGH::3′Thia4. Proceeding in the 5′ to 3′ direction, bold, lowercase sequences represent genomic DNA from CHK22 that permit targeted integration at the Thia4 locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR that enables amplification of the VbolLPAAT2 gene, is indicated by uppercase underlined text followed by a linker, indicated by lowercase, bold italics, which contains a C. vulgaris nitrate reductase 3′-UTR. The P. moriformis PmG3PDH promoter, indicated by boxed italicized text, drive the expression of the VbolLPAAT2. The initiator ATG and terminator TGA codons of the VbolLPAAT2 are indicated by uppercase, bold italics, while the remainder of the gene is indicated by bold italics. The PmPGH 3′-UTR is indicated by lowercase underlined text followed by the CHK22 Thia4 genomic region indicated by bold, lowercase text.

Table 83 shows the fatty acid/TAG profiles of eight primary transformants of CHK22 transformed with pCHK1214. The P. moriformis base strain CHK22 is shown as a non-transgenic control. Strains were grown for 96 hrs in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above

TABLE 83
Screen of primary transformants of CHK22 with pCHK1214.

| | FAME Profiles | | | | | | | | | TAG Profile |
|---|---|---|---|---|---|---|---|---|---|---|
| | | | | | | | C18:3 | | | m/z 577/603 |
| Sample | C12:0 | C14:0 | C16:0 | C18:0 | C18:1 | C18:2 | alpha | C20:0 | C20:1 | (OP:OO) |
| pCHK1214-1 | 0.04 | 1.37 | 26.66 | 2.88 | 60.78 | 6.30 | 0.40 | 0.20 | 0.04 | 1.6 |
| pCHK1214-2 | 0.04 | 1.47 | 29.88 | 2.83 | 56.72 | 6.87 | 0.53 | 0.21 | 0.00 | 2 |
| pCHK1214-3 | 0.03 | 1.28 | 27.68 | 2.94 | 59.10 | 6.91 | 0.49 | 0.22 | 0.00 | 2 |
| pCHK1214-4 | 0.03 | 1.17 | 25.97 | 2.91 | 61.34 | 6.48 | 0.49 | 0.23 | 0.00 | 1.6 |
| pCHK1214-5 | 0.03 | 1.26 | 27.50 | 2.78 | 59.51 | 6.86 | 0.46 | 0.19 | 0.03 | 2.1 |
| pCHK1214-6 | 0.03 | 1.31 | 27.66 | 2.85 | 58.78 | 7.24 | 0.52 | 0.21 | 0.03 | 2 |
| pCHK1214-7 | 0.03 | 1.28 | 27.57 | 2.90 | 59.77 | 6.39 | 0.48 | 0.21 | 0.00 | 1.7 |
| pCHK1214-8 | 0.03 | 1.31 | 27.93 | 2.98 | 58.75 | 6.92 | 0.50 | 0.22 | 0.03 | 2.1 |
| CHK22 Control | 0.04 | 1.4 | 25.8 | 2.69 | 61.83 | 6.51 | 0.36 | 0.16 | 0 | 1.5 |

### Example 40: Expressing Pandorina morum (PmorLPAAT2) in P. Moriformis Strain Chk22 Using the PmG3PDH Promoter

PmorLPAAT2 was introduced into a P. moriformis strain CHK22. The expression construct pCHK1215 contained 5′ and 3′ homology arms to permit its targeted integration into the genome and is shown in Table 84.

TABLE 84
Integrative sequences for the transformation of P. moriformis with pCHK1215
encoding PmorLPAAT2.

| SEQ | |
|---|---|
| ID | |
| NO: | Nucleotide Sequence |
| 70 | ccctcaactgcgacgctgggaaccttctccgggcaggcgatgtgcgtgggtttgcctccttggcacggctctacaccgtcgagtacgcc |
| | atgaggcggtgatggctgtgtcggttgccacttcgtccagagacggcaagtcgtccatcctctgcgtgtgtggcgcgacgctgcagca |
| | gtccctctgcagcagatgagcgtgactttggccatttcacgcactegagtgtacacaatccatttttcttaaagcaaatgactgctgatt |
| | gaccagatactgtaacgctgatttcgctccagatcgcacagatagcgaccatgttgctgcgtctgaaaatctggattcegaattegacc |
| | ctggcgctccatccatgcaacagatggcgacacttgttacaattcctgtcacccateggcatggagcaggtccacttagattcccgatc |
| | acccacgcacatctcgctaatagtcattcgttcgtgtcttcgatcaatctcaagtgagtgtgcatggatcttggttgacgatgcggtatg |
| | ggtttgcgccgctggctgcagggtctgcccaaggcaagctaacccagctcctctccccgacaatactctcgcaggcaaagccggtca |
| | cttgccttccagattgccaataaactcaattatggcctctgtcatgccatccatgggtctgatgaatggtcacgctcgtgtcctgaccgtt |
| | 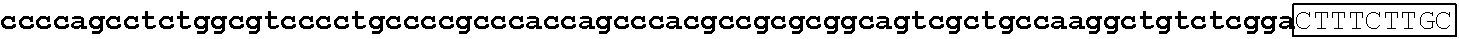 |
| | 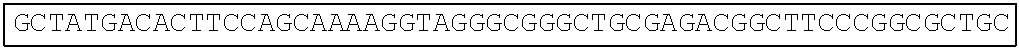 |
| | 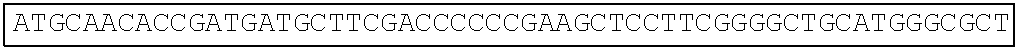 |
| | 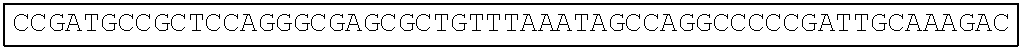 |
| | 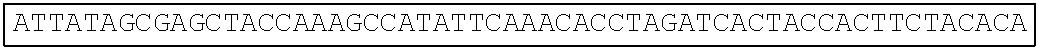 |
| | 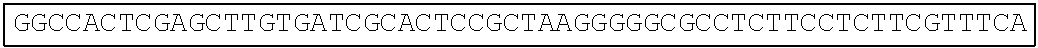 |
| | 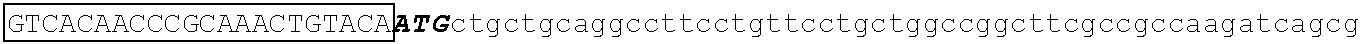 |
| | cctccatgacgaacgagacgtccgaccgccccctggtgcacttcacccccaacaagggctggatgaacgaccccaacggcctgtggta |
| | cgacgagaaggacgccaagtggcacctgtacttccagtacaacccgaacgacaccgtctgggggacgcccttgttctggggccacgcc |
| | acgtccgacgacctgaccaactgggaggaccagcccatcgccatcgccccgaagcgcaacgactccggcgccttctccggctccatgg |
| | tggtggactacaacaacacctccggcttcttcaacgacaccatcgacccgcgccagcgctgcgtggccatctggacctacaacaccccg |
| | gagtccgaggagcagtacatctcctacagcctggacggcggctacaccttcaccgagtaccagaagaaccccgtgctggccgccaact |
| | ccacccagttccgcgacccgaaggtcttctggtacgagccctcccagaagtggatcatgaccgcggccaagtcccaggactacaagat |
| | cgagatctactcctccgacgacctgaagtcctggaagctggagtccgcgttcgccaacgagggcttcctcggctaccagtacgagtgccc |
| | cggcctgatcgaggtccccaccgagcaggaccccagcaagtcctactgggtgatgttcatctccatcaaccccggcgccccggccggc |
| | ggctccttcaaccagtacttcgtcggcagcttcaacggcacccacttcgaggccttcgacaaccagtcccgcgtggtggacttcggcaag |
| | gactactacgccctgcagaccttcttcaacaccgacccgacctacgggagcgccctgggcatcgcgtgggcctccaactgggagtactc |
| | cgccttcgtgcccaccaacccctggcgctcctccatgtccctcgtgcgcaagttctccctcaacaccgagtaccaggccaacccggagac |
| | ggagctgatcaacctgaaggccgagccgatcctgaacatcagcaacgccggcccctggagccggttcgccaccaacaccacgttgac |
| | gaaggccaacagctacaacgtcgacctgtccaacagcaccggcaccctggagttcgagctggtgtacgccgtcaacaccacccagac |
| | gatctccaagtccgtgttcgcggacctctccctctggttcaagggcctggaggaccccgaggagtacctccgcatgggcttcgaggtgtcc |
| | gcgtcctccttcttcctggaccgcgggaacagcaaggtgaagttcgtgaaggagaacccctacttcaccaaccgcatgagcgtgaacaa |
| | ccagcccttcaagagcgagaacgacctgtcctactacaaggtgtacggcttgctggaccagaacatcctggagctgtacttcaacgacg |
| | gcgacgtcgtgtccaccaacacctacttcatgaccaccgggaacgccctgggctccgtgaacatgacgacgggggtggacaacctgttc |
| | tacatcgacaagttccaggtgcgcgaggtcaagtgactTAAGACGCCCGCGCGGCGCACCTGACCTGTTCT |
| | CTCGAGGGCGCCTGTTCTGCCTTGCGAAACAAGCCCCTGGAGCATGCGTGCATGATCG |
| | TCTCTGGCGCCCCGCCGCGCGGTTTGTCGCCCTCGCGGGCGCCGCGGCCGCGGGGGC |
| | GCATTGAAATTGTTGCAAACCCCACCTGACAGATTGAGGGCCCAGGCAGGAAGGCGT |
| | TGAGATGGAGGTACAGGAGTCAAGTAACTGAAAGTTTTTATGATAACTAACAACAAA |
| | GGGTCGTTTCTGGCCAGCGAATGACAAGAACAAGATTCCACATTTCCGTGTAGAGGC |
| | TTGCCATCGAATGTGAGCGGGGGGCCGCGGACCCGACAAAACCCTTACGACGTGGT |
| | AAGAAAAACGTGGCGGGCACTGTCCCTGTAGCCTGAAGACCAGCAGGAGACGATCG |
| | GAAGCATCACAGCACAACCGGTgcagcagcagcteggatagtatcgacacactctggacgctggtcgtgtgatggac |
| | tgttgccgccacacttgctgccttgacctgtgaatatccctgccgcttttatcaaacagcctcagtgtgtttgatcttgtgtgtacgcgcttttgc |
| | gagttgctagctgcttgtgctatttgegaataccacccccagcatccccttccctcgtttcatategcttgcatcccaaccgcaacttatctacgc |
| | tgtcctgctatccctcagegctgctcctgctcctgctcactgcccctcgcacagccttggtttgggctccgcctgtattctcctggtactgcaacc |
| | 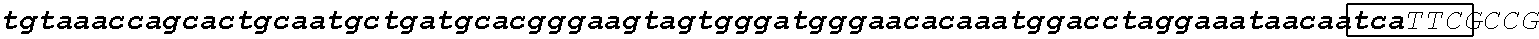 |
| | 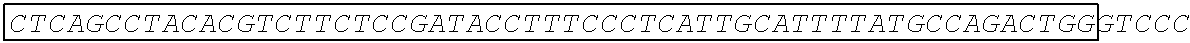 |
| | 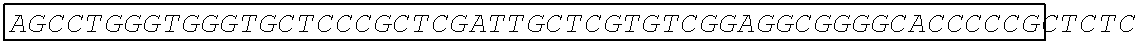 |
| | 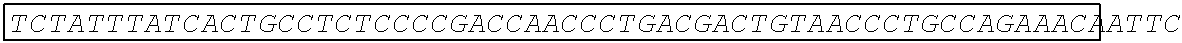 |
| | 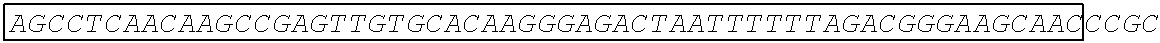 |
| | 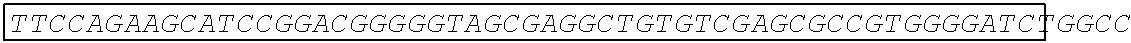 |
| | 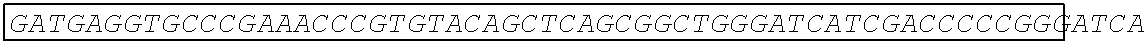 |
| | 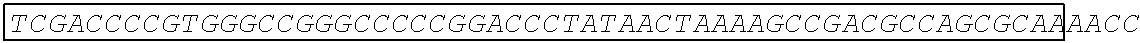 |
| | 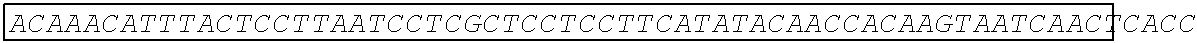 |
| | 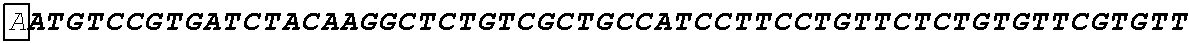 |
| | TTACTGGTCCCTGCCTATCTTCGCCATTCCATACCGCATCCGCTTCGCTAACGTGGGCA |
| | AGCGGAACGACATGCTCGACTGGGCGAAGGCTCTGGTCGGCTTCTTTCGCGTGACGGT |
| | CCTGAAGCCCGGCGCGACCTCCGTGTACCGCGGGGGTCCGTGCCTGTACCTCTGCAAC |
| | CATCGCTCCTGGGCCGACTTTTTCATCGACGTGTACCTGACCCAGGGCCAGTCGGCCCT |
| | CATGTCGCGGTGGCTGGTGTTCTTTGTCTTTCCCGTGTTCTGTACCTCCTGCCTGATCCT |
| | CCGCGGCATCGTCCTCTTTAAGCGGGGCACGATCCTGGACAAGGAGGCGTTTAACGCC |
| | TGGCTCGATGCTACGCGCGCCCGAAGCATCGTGCCGGGCCTGGTGGTCTACCCGGAGG |
| | GGCATCGGTCCCTGAAGCCGACCTCGCTGCCGCTGAAGCGCGGGATGATCCACTACGC |
| | CTTCTCTCGGCGCCTGCCAGTCCAACTGATCGTGACCTCGGGCAAGGAGCATGTGCTGA |
| | GCGAGAAGACGATGTCGGTCAAGTTTGGCAAGACCCTCGTGACCTCGTACTCGAAGCC |
| | GCTGAAGCCTAGCGATTACGCGACGTTTGATGCCTTCATGGCCGACATCCAGTCGAACT |
| | GGGACCAGTGCTGGAAGGAGGCGTACACCGCTCCCGCTCGCGGCATGCCAGAGGTCTC |
| | CATGGCGGGCGCGCACTCTTACGCCTACCCACGCCTGACCTGGTGCCTCCAGCTCGCC |
| | ATCACCGTCGTGTCCATCGTGATCCTGGGAGGGGTGCTGGCGGGCTCGTGGCGGGGCG |
| | CCGCGGCCGTCCTCGCTGCCGCGGGCCCCGTCGGCCAACAGGTGCTCGTCTCCCTGCT |
| | CGCCCTGTGGGCGGGGGTCTCCCTCCTGCGATCGTTCCTCTGAgtaccacgcccgegeggcgcacct |
| | gacctgttctctcgagggcgcctgttctgccttgcgaaacaagcccctggagcatgcgtgcatgatcgtctctggcgccccgccgcgcggttt |
| | gtcgccctcgcgggcgccccggccgcgggggcgcattgaaattgttgcaaaccccacctgacagattgagggcccaggcaggaaggcgtt |
| | gagatggaggtacaggagtcaagtaactgaaagtttttatgataactaacaacaaagggtcgtttctggccagcgaatgacaagaacaagattc |
| | cacatttccgtgtagaggcttgccatcgaatgtgagcggggggccgcggacccgacaaaacccttacgacgtggtaagaaaaacgtggcg |
| | ggcactgtccctgtagcctgaagaccagcaggagacgatcggaagcatcacagcacaggccggcccgcgtctcgaacagagcgcggcg |
| | gccgcatcgatacctgcaggacagcgccatgccacgccctttgatggcttcaagtacgattacggtgttggattgtgtgtttgttgcgtag |
| | tgtgcatggtttagaataatacacttgatttcttgctcacggcaatctcggcttgtccgcaggttcaaccccattteggagtctcaggtca |
| | gccgcgcaatgaccagccgctacttcaaggacttgcacgacaacgccgaggtgagctatgtttaggacttgattggaaattgtcgtcg |
| | acgcatattegcgctccgcgacagcacccaagcaaaatgtcaagtgcgttccgatttgcgtccgcaggtcgatgttgtgatcgtcggc |
| | gccggatccgccggtctgtcctgcgcttacgagctgaccaagcaccctgacgtccgggtacgcgagctgagattcgattagacataa |
| | attgaagattaaacccgtagaaaaatttgatggtcgcgaaactgtgctcgattgcaagaaattgatcgtcctccactccgcaggtcgc |
| | catcatcgagcagggcgttgctcccggcggcggcgcctggctggggggacagctgttctcggccatgtgtgtacgtagaaggatgaa |
| | tttcagctggttttcgttgcacagctgtttgtgcatgatttgtttcagactattgttgaatgtttttagatttcttaggatgcatgatttgtctg |
| | catgcgact |

The construct can be written as 5′ Thia4::CrTUB2:ScSUC2:PmPGH:CvNR:PmG3PDH:PmorLPAAT2:PmPGH::3′ Thia4. Proceeding in the 5′ to 3′ direction, bold, lowercase sequences represent genomic DNA from CHK22 that permit targeted integration at the Thia4 locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR that enables amplification of the PmorLPAAT2 gene, is indicated by uppercase underlined text followed by a linker, indicated by lowercase, bold italics, which contains a C. vulgaris nitrate reductase 3′-UTR. The P. moriformis PmG3PDH promoter, indicated by boxed italicized text, drive the expression of the PmorLPAAT2. The initiator ATG and terminator TGA codons of the PmorLPAAT2 are indicated by uppercase, bold italics, while the remainder of the gene is indicated by bold italics. The PmPGH 3′-UTR is indicated by lowercase underlined text followed by the CHK22 Thia4 genomic region indicated by bold, lowercase text.

Table 85 shows the fatty acid/TAG profiles of seven primary transformants of CHK22 transformed with pCHK1215. The P. moriformis base strain CHK22 is shown as a non-transgenic control. Strains were grown for 120 hrs in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above

TABLE 85
Screen of primary transformants of CHK22 with pCHK1215.

| | FAME Profiles | | | | | | | | | TAG Profile |
|---|---|---|---|---|---|---|---|---|---|---|
| | | | | | | | C18:3 | | | m/z 577/603 |
| Sample | C12:0 | C14:0 | C16:0 | C18:0 | C18:1 | C18:2 | alpha | C20:0 | C20:1 | (OP:OO) |
| pCHK1215-1 | 0.04 | 1.28 | 27.52 | 2.88 | 59.31 | 6.54 | 0.46 | 0.25 | 0.00 | 1.96 |
| pCHK1215-2 | 0.04 | 1.37 | 28.49 | 3.23 | 55.59 | 8.12 | 0.90 | 0.40 | 0.00 | 1.65 |
| pCHK1215-3 | 0.04 | 1.34 | 27.96 | 2.94 | 58.55 | 6.86 | 0.49 | 0.27 | 0.00 | 1.56 |
| pCHK1215-4 | 0.04 | 1.37 | 28.10 | 3.29 | 56.48 | 7.72 | 0.84 | 0.40 | 0.00 | 1.49 |
| pCHK1215-5 | 0.04 | 1.43 | 27.16 | 2.28 | 60.66 | 6.01 | 0.28 | 0.17 | 0.00 | 1.68 |
| pCHK1215-6 | 0.04 | 1.55 | 28.28 | 2.96 | 56.31 | 8.36 | 0.59 | 0.31 | 0.00 | 1.55 |
| pCHK1215-7 | 0.04 | 1.35 | 28.28 | 2.87 | 57.63 | 7.31 | 0.52 | 0.27 | 0.00 | 1.63 |
| CHK22 Control | 0.04 | 1.27 | 27.29 | 2.45 | 59.08 | 7.45 | 0.54 | 0.23 | 0.00 | 1.5 |

### Example 41: Expressing Volvox carteri f. weismannia (VcarfLPAAT2) in P. moriformis Strain CHK22 Using the PmG3PDH Promoter

VcarfLPAAT2 was introduced into a P. moriformis strain CHK22. The expression construct pCHK1216 contained 5′ and 3′ homology arms to permit its targeted integration into the genome and is shown in Table 86.

TABLE 86
Integrative sequences for the transformation of P. moriformis with pCHK1216
encoding VcarfLPAAT2.

| SEQ | |
|---|---|
| ID | |
| NO: | Nucleotide Sequence |
| 71 | ccctcaactgcgacgctgggaaccttctccgggcaggcgatgtgcgtgggtttgcctccttggcacggctctacaccgtegagtacgcc |
| | atgaggcggtgatggctgtgtcggttgccacttcgtccagagacggcaagtcgtccatcctctgcgtgtgtggcgcgacgctgcagca |
| | gtccctctgcagcagatgagcgtgactttggccatttcacgcactcgagtgtacacaatccatttttcttaaagcaaatgactgctgatt |
| | gaccagatactgtaacgctgatttcgctccagategcacagatagcgaccatgttgctgcgtctgaaaatctggattccgaattcgacc |
| | ctggcgctccatccatgcaacagatggcgacacttgttacaattcctgtcacccateggcatggagcaggtccacttagattcccgatc |
| | acccacgcacatctcgctaatagtcattcgttcgtgtcttcgatcaatctcaagtgagtgtgcatggatcttggttgacgatgcggtatg |
| | ggtttgcgccgctggctgcagggtctgcccaaggcaagctaacccagctcctctccccgacaatactctcgcaggcaaagceggtca |
| | cttgccttccagattgccaataaactcaattatggcctctgtcatgccatccatgggtctgatgaatggtcacgctcgtgtcctgaccgtt |
| | 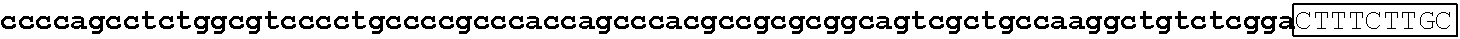 |
| | 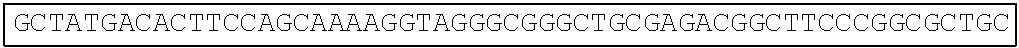 |
| | 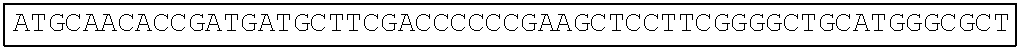 |
| | 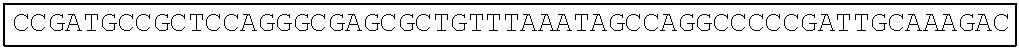 |
| | 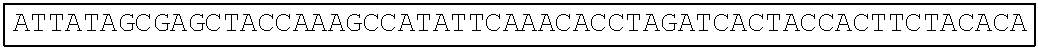 |
| | 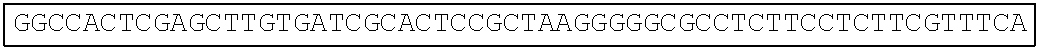 |
| | 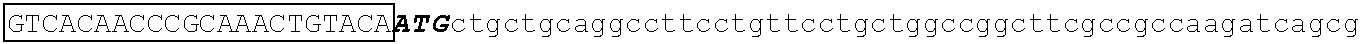 |
| | cctccatgacgaacgagacgtccgaccgccccctggtgcacttcacccccaacaagggctggatgaacgaccccaacggcctgtggta |
| | cgacgagaaggacgccaagtggcacctgtacttccagtacaacccgaacgacaccgtctgggggacgcccttgttctggggccacgcc |
| | acgtccgacgacctgaccaactgggaggaccagcccatcgccatcgccccgaagcgcaacgactccggcgccttctccggctccatgg |
| | tggtggactacaacaacacctccggcttcttcaacgacaccatcgacccgcgccagcgctgcgtggccatctggacctacaacaccccg |
| | gagtccgaggagcagtacatctcctacagcctggacggcggctacaccttcaccgagtaccagaagaaccccgtgctggccgccaact |
| | ccacccagttccgcgacccgaaggtcttctggtacgagccctcccagaagtggatcatgaccgcggccaagtcccaggactacaagat |
| | cgagatctactcctccgacgacctgaagtcctggaagctggagtccgcgttcgccaacgagggcttcctcggctaccagtacgagtgccc |
| | cggcctgatcgaggtccccaccgagcaggaccccagcaagtcctactgggtgatgttcatctccatcaaccccggcgccccggccggc |
| | ggctccttcaaccagtacttcgtcggcagcttcaacggcacccacttcgaggccttcgacaaccagtcccgcgtggtggacttcggcaag |
| | gactactacgccctgcagaccttcttcaacaccgacccgacctacgggagcgccctgggcatcgcgtgggcctccaactgggagtactc |
| | cgccttcgtgcccaccaacccctggcgctcctccatgtccctcgtgcgcaagttctccctcaacaccgagtaccaggccaacccggagac |
| | ggagctgatcaacctgaaggccgagccgatcctgaacatcagcaacgccggcccctggagccggttcgccaccaacaccacgttgac |
| | gaaggccaacagctacaacgtcgacctgtccaacagcaccggcaccctggagttcgagctggtgtacgccgtcaacaccacccagac |
| | gatctccaagtccgtgttcgcggacctctccctctggttcaagggcctggaggaccccgaggagtacctccgcatgggcttcgaggtgtcc |
| | gcgtcctccttcttcctggaccgcgggaacagcaaggtgaagttcgtgaaggagaacccctacttcaccaaccgcatgagcgtgaacaa |
| | ccagcccttcaagagcgagaacgacctgtcctactacaaggtgtacggcttgctggaccagaacatcctggagctgtacttcaacgacg |
| | gcgacgtcgtgtccaccaacacctacttcatgaccaccgggaacgccctgggctccgtgaacatgacgacgggggtggacaacctgttc |
| | tacatcgacaagttccaggtgcgcgaggtcaagtgactTAAGACGCCCGCGCGGCGCACCTGACCTGTTCT |
| | CTCGAGGGCGCCTGTTCTGCCTTGCGAAACAAGCCCCTGGAGCATGCGTGCATGATCG |
| | TCTCTGGCGCCCCGCCGCGCGGTTTGTCGCCCTCGCGGGCGCCGCGGCCGCGGGGGC |
| | GCATTGAAATTGTTGCAAACCCCACCTGACAGATTGAGGGCCCAGGCAGGAAGGCGT |
| | TGAGATGGAGGTACAGGAGTCAAGTAACTGAAAGTTTTTATGATAACTAACAACAAA |
| | GGGTCGTTTCTGGCCAGCGAATGACAAGAACAAGATTCCACATTTCCGTGTAGAGGC |
| | TTGCCATCGAATGTGAGCGGGCGGGCCGCGGACCCGACAAAACCCTTACGACGTGGT |
| | AAGAAAAACGTGGCGGGCACTGTCCCTGTAGCCTGAAGACCAGCAGGAGACGATCG |
| | GAAGCATCACAGCACAACCGGTgcagcagcagctcggatagtatcgacacactctggacgctggtcgtgtgatggac |
| | tgttgccgccacacttgctgccttgacctgtgaatatccctgccgcttttatcaaacagcctcagtgtgtttgatcttgtgtgtacgcgcttttgc |
| | gagttgctagctgcttgtgctatttgegaataccacccccagcatccccttccctcgtttcatategcttgcatcccaaccgcaacttatctacgc |
| | tgtcctgctatccctcagegctgctcctgctcctgctcactgcccctcgcacagccttggtttgggctccgcctgtattctcctggtactgcaacc |
| | 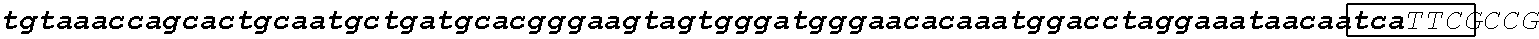 |
| | 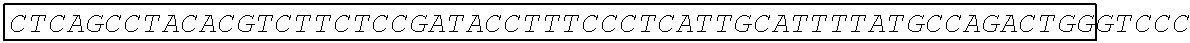 |
| | 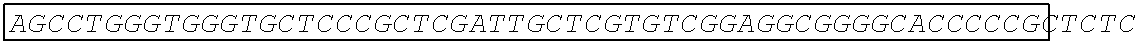 |
| | 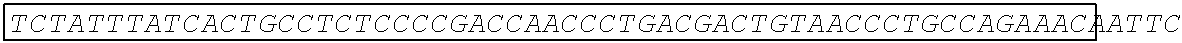 |
| | 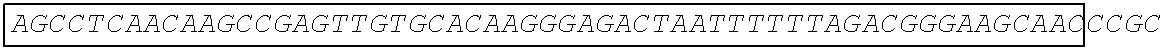 |
| | 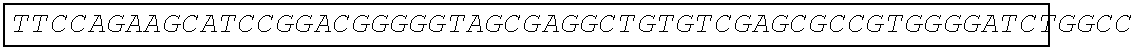 |
| | 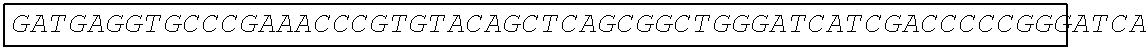 |
| | 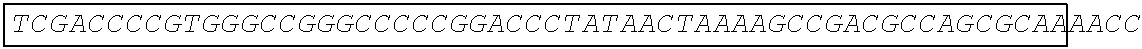 |
| | 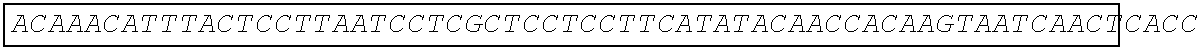 |
| | 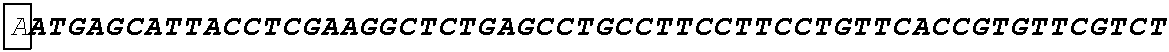 |
| | TCTACTGGTCCCTGCCTCTCTTCGCCATTATCTACCGCATCCGCTTTGCCAACGTGGGC |
| | AAGCGGAACGATATGCTGGATTGGGCTAAGGCGCTCGTCGCCTTTTTCCGCGTCACCGT |
| | CCTGAAGCAGGGTGAGATGTCCCTGTACCGCGGAGGCCGCTGCCTGTACCTGTGCAAC |
| | CACCGCTCCTGGGCCGACTTTTTCATCGACACCTACCTGACGGAGGGACGCGCCGCGC |
| | TCATGTCTCGGTGGATGGTGTTTTTCGCCTTTCCCGTGTTTTCCACGTCCGTCCTGATCC |
| | TGCGCGGCATCGTCCTGTTTAAGCGGGGCCATATCGCCGACAAGGAGGCCTTTAACTCT |
| | TGGCTCGACGCCACCCTGCGGAAGTCGACGGTGCCAGCCCTGCTCGTCTACCCGGAGG |
| | GACACCGCTCTCTGAAGCCGCGCTCCCTGCCCCTCAAGCGCGGTATGCTGCATTTCGCG |
| | TTCTCCCGCCAGCTCGCGGTGCAGATGGTCATCACGCGAGGCAAGGAGGAGGTGCTGA |
| | GCGAGAAGACGTGCTCGGCCCGCTGGGGCCGCACCCTGGTCACCACGTACTCCAAGGT |
| | GATCAAGCCCACGGACTACGACTCCTTTGACGCTTTTTTCAACGAGATCCAAACCACGT |
| | GGGACGCCTGCTGGCAACTGTCGTACCACCAGCCCACCGAGGGCGTGCCCCAACTGTC |
| | GCTGAAGGGCGCGCACGAGTACGACTACCCGGTGAACATGTGGTGGCTGCAACTGGGC |
| | GTCACGCTCCTGTCCATCACGATCCTGGTGGCGGTCCTGTACGGGAGCTGGGTGGCGC |
| | TGCGCGCGGGCGTCTCTTCCCTGGGCAGCTCCATCACGCAACAGGCGGTGGTCCTGGG |
| | CGTGGCGGGCTGGCTCGGCGTGTCCCTCCTGCGGTCGTTCCTGTGAgtaccacgcccgcgcggc |
| | gcacctgacctgttctctcgagggcgcctgttctgccttgcgaaacaagcccctggagcatgcgtgcatgatcgtctctggogccccgccgcg |
| | cggtttgtcgccctcgcgggcgccccggccgcgggggcgcattgaaattgttgcaaaccccacctgacagattgagggcccaggcaggaa |
| | ggcgttgagatggaggtacaggagtcaagtaactgaaagtttttatgataactaacaacaaagggtcgtttctggccagcgaatgacaagaac |
| | aagattccacatttccgtgtagaggcttgccatcgaatgtgagcgggcgggccgeggacccgacaaaacccttacgacgtggtaagaaaaa |
| | cgtggcgggcactgtccctgtagcctgaagaccagcaggagacgatcggaagcatcacagcacaggccggcccgcgtctcgaacagagc |
| | gcggcggccgcatcgatacctgcaggacagcgccatgccacgccctttgatggcttcaagtacgattacggtgttggattgtgtgtttgtt |
| | gcgtagtgtgcatggtttagaataatacacttgatttcttgctcacggcaatctcggcttgtccgcaggttcaaccccattteggagtctc |
| | aggtcagccgcgcaatgaccagccgctacttcaaggacttgcacgacaacgccgaggtgagctatgtttaggacttgattggaaatt |
| | gtcgtcgacgcatattegcgctccgcgacagcacccaagcaaaatgtcaagtgcgttccgatttgcgtccgcaggtcgatgttgtgatc |
| | gtcggcgccggatccgccggtctgtcctgcgcttacgagctgaccaagcaccctgacgtccgggtacgcgagctgagattcgattaga |
| | cataaattgaagattaaacccgtagaaaaatttgatggtcgcgaaactgtgctcgattgcaagaaattgatcgtcctccactccgcag |
| | gtcgccatcatcgagcagggcgttgctcccggcggcggcgcctggctggggggacagctgttctcggccatgtgtgtacgtagaagg |
| | atgaatttcagctggttttcgttgcacagctgtttgtgcatgatttgtttcagactattgttgaatgtttttagatttcttaggatgcatgattt |
| | gtctgcatgcgact |

The construct can be written as 5′Thia4::CrTUB2:ScSUC2:PmPGH:CvNR:PmG3PDH:VcarfLPAAT2:PmPGH::3′Thia4. Proceeding in the 5′ to 3′ direction bold, lowercase sequences represent genomic DNA from CHK22 that permit targeted integration at the Thia4 locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR that enables amplification of the VcarfLPAAT2 gene, is indicated by uppercase underlined text followed by a linker, indicated by lowercase, bold italics, which contains a C. vulgaris nitrate reductase 3′-UTR. The P. moriformis PmG3PDH promoter, indicated by boxed italicized text, drive the expression of the VcarfLPAAT2. The initiator ATG and terminator TGA codons of the VcarfLPAAT2 are indicated by uppercase, bold italics, while the remainder of the gene is indicated by bold italics. The PmPGH 3′-UTR is indicated by lowercase underlined text followed by the CHK22 Thia4 genomic region indicated by bold, lowercase text.

Table 87 shows the fatty acid/TAG profiles of seven primary transformants of CHK22 transformed with pCHK1216. The P. moriformis base strain CHK22 is shown as a non-transgenic control. Strains were grown for 120 hrs in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above

TABLE 87
Screen of primary transformants of CHK22 with pCHK1216.

| | FAME Profiles | | | | | | | | | TAG Profile |
|---|---|---|---|---|---|---|---|---|---|---|
| | | | | | | | C18:3 | | | m/z 577/603 |
| Sample | C12:0 | C14:0 | C16:0 | C18:0 | C18:1 | C18:2 | alpha | C20:0 | C20:1 | (OP:OO) |
| pCHK1216-1 | 0.04 | 1.34 | 28.27 | 2.83 | 57.48 | 7.53 | 0.49 | 0.25 | 0.00 | 3.20 |
| pCHK1216-2 | 0.04 | 1.36 | 28.16 | 2.76 | 57.88 | 7.41 | 0.47 | 0.24 | 0.00 | 3.25 |
| pCHK1216-3 | 0.04 | 1.40 | 28.56 | 2.78 | 56.79 | 8.04 | 0.50 | 0.25 | 0.00 | 3.38 |
| pCHK1216-4 | 0.04 | 1.37 | 28.36 | 2.82 | 57.35 | 7.67 | 0.50 | 0.25 | 0.00 | 3.11 |
| pCHK1216-5 | 0.04 | 1.31 | 28.00 | 2.96 | 58.66 | 6.65 | 0.49 | 0.27 | 0.00 | 1.56 |
| pCHK1216-6 | 0.04 | 1.31 | 26.65 | 2.34 | 58.33 | 8.80 | 0.50 | 0.22 | 0.00 | 3.36 |
| pCHK1216-7 | 0.04 | 1.40 | 28.38 | 2.67 | 57.13 | 7.93 | 0.50 | 0.24 | 0.00 | 3.10 |
| CHK22 Control | 0.04 | 1.27 | 27.29 | 2.45 | 59.08 | 7.45 | 0.54 | 0.23 | 0.00 | 1.5 |

### Example 42: Expressing Eudorina cylindrica (EcylLPAAT2) in P. moriformis Strain CHK22 Using the PmG3PDH Promoter

EcylLPAAT2 was introduced into a P. moriformis strain CHK22. The expression construct pCHK1217 contained 5′ and 3′ homology arms to permit its targeted integration into the genome and is shown in Table 88.

TABLE 88
Integrative sequences for the transformation of P. moriformis with pCHK1217
encoding EcyILPAAT2.

| SEQ | |
|---|---|
| ID | |
| NO: | Nucleotide Sequence |
| 72 | ccctcaactgcgacgctgggaaccttctccgggcaggcgatgtgcgtgggtttgcctccttggcacggctctacaccgtcgagtacgcc |
| | atgaggcggtgatggctgtgtcggttgccacttcgtccagagacggcaagtcgtccatcctctgcgtgtgtggcgcgacgctgcagca |
| | gtccctctgcagcagatgagcgtgactttggccatttcacgcactcgagtgtacacaatccatttttcttaaagcaaatgactgctgatt |
| | gaccagatactgtaacgctgatttcgctccagatcgcacagatagcgaccatgttgctgcgtctgaaaatctggattccgaattcgacc |
| | ctggcgctccatccatgcaacagatggcgacacttgttacaattcctgtcacccateggcatggagcaggtccacttagattcccgatc |
| | acccacgcacatctcgctaatagtcattcgttcgtgtcttcgatcaatctcaagtgagtgtgcatggatcttggttgacgatgcggtatg |
| | ggtttgcgccgctggctgcagggtctgcccaaggcaagctaacccagctcctctccccgacaatactctcgcaggcaaagccggtca |
| | cttgccttccagattgccaataaactcaattatggcctctgtcatgccatccatgggtctgatgaatggtcacgctcgtgtcctgaccgtt |
| | 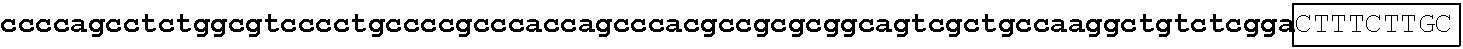 |
| | 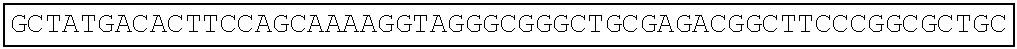 |
| | 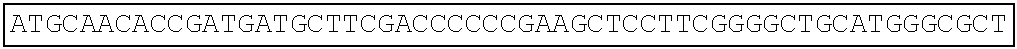 |
| | 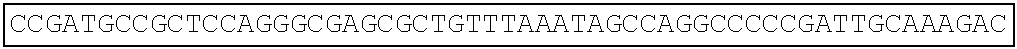 |
| | 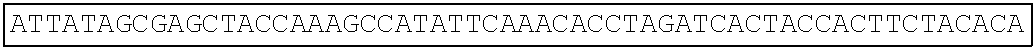 |
| | 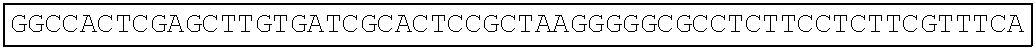 |
| | 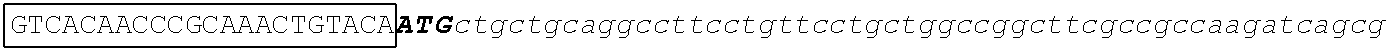 |
| | cctccatgacgaacgagacgtccgaccgccccctggtgcacttcacccccaacaagggctggatgaacgaccccaacggcctgtggta |
| | cgacgagaaggacgccaagtggcacctgtacttccagtacaacccgaacgacaccgtctgggggacgcccttgttctggggccacgcc |
| | acgtccgacgacctgaccaactgggaggaccagcccatcgccatcgccccgaagcgcaacgactccggcgccttctccggctccatgg |
| | tggtggactacaacaacacctccggcttcttcaacgacaccatcgacccgcgccagcgctgcgtggccatctggacctacaacaccccg |
| | gagtccgaggagcagtacatctcctacagcctggacggcggctacaccttcaccgagtaccagaagaaccccgtgctggccgccaact |
| | ccacccagttccgcgacccgaaggtcttctggtacgagccctcccagaagtggatcatgaccgcggccaagtcccaggactacaagat |
| | cgagatctactcctccgacgacctgaagtcctggaagctggagtccgcgttcgccaacgagggcttcctcggctaccagtacgagtgccc |
| | cggcctgatcgaggtccccaccgagcaggaccccagcaagtcctactgggtgatgttcatctccatcaaccccggcgccccggccggc |
| | ggctccttcaaccagtacttcgtcggcagcttcaacggcacccacttcgaggccttcgacaaccagtcccgcgtggtggacttcggcaag |
| | gactactacgccctgcagaccttcttcaacaccgacccgacctacgggagcgccctgggcatcgcgtgggcctccaactgggagtactc |
| | cgccttcgtgcccaccaacccctggcgctcctccatgtccctcgtgcgcaagttctccctcaacaccgagtaccaggccaacccggagac |
| | ggagctgatcaacctgaaggccgagccgatcctgaacatcagcaacgccggcccctggagccggttcgccaccaacaccacgttgac |
| | gaaggccaacagctacaacgtcgacctgtccaacagcaccggcaccctggagttcgagctggtgtacgccgtcaacaccacccagac |
| | gatctccaagtccgtgttcgcggacctctccctctggttcaagggcctggaggaccccgaggagtacctccgcatgggcttcgaggtgtcc |
| | gcgtcctccttcttcctggaccgcgggaacagcaaggtgaagttcgtgaaggagaacccctacttcaccaaccgcatgagcgtgaacaa |
| | ccagcccttcaagagcgagaacgacctgtcctactacaaggtgtacggcttgctggaccagaacatcctggagctgtacttcaacgacg |
| | gcgacgtcgtgtccaccaacacctacttcatgaccaccgggaacgccctgggctccgtgaacatgacgacgggggtggacaacctgttc |
| | tacatcgacaagttccaggtgcgcgaggtcaagtgactTAAGACGCCCGCGCGGCGCACCTGACCTGTTCT |
| | CTCGAGGGCGCCTGTTCTGCCTTGCGAAACAAGCCCCTGGAGCATGCGTGCATGATCG |
| | TCTCTGGCGCCCCGCCGCGCGGTTTGTCGCCCTCGCGGGCGCCGCGGCCGCGGGGGC |
| | GCATTGAAATTGTTGCAAACCCCACCTGACAGATTGAGGGCCCAGGCAGGAAGGCGT |
| | TGAGATGGAGGTACAGGAGTCAAGTAACTGAAAGTTTTTATGATAACTAACAACAAA |
| | GGGTCGTTTCTGGCCAGCGAATGACAAGAACAAGATTCCACATTTCCGTGTAGAGGC |
| | TTGCCATCGAATGTGAGCGGGCGGGCCGCGGACCCGACAAAACCCTTACGACGTGGT |
| | AAGAAAAACGTGGCGGGCACTGTCCCTGTAGCCTGAAGACCAGCAGGAGACGATCG |
| | GAAGCATCACAGCACAACCGGTgcagcagcagctcggatagtatcgacacactctggacgctggtcgtgtgatggac |
| | tgttgccgccacacttgctgccttgacctgtgaatatccctgccgcttttatcaaacagcctcagtgtgtttgatcttgtgtgtacgcgcttttgc |
| | gagttgctagctgcttgtgctatttgcgaataccacccccagcatccccttccctegtttcatategcttgcatcccaaccgcaacttatctacgc |
| | tgtcctgctatccctcagcgctgctcctgctcctgctcactgcccctcgcacagccttggtttgggctccgcctgtattctcctggtactgcaacc |
| | 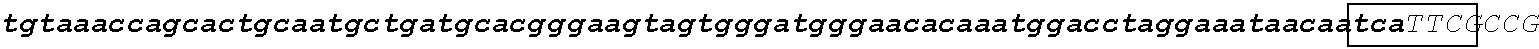 |
| | 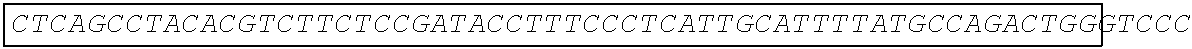 |
| | 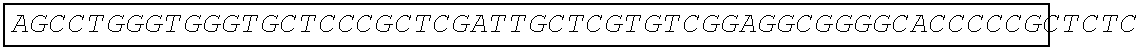 |
| | 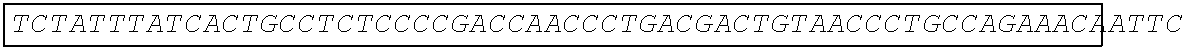 |
| | 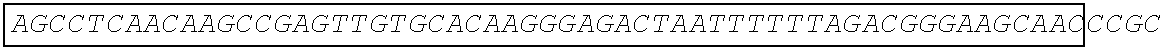 |
| | 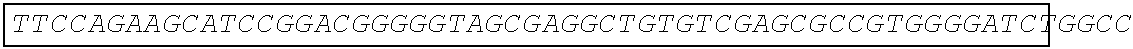 |
| | 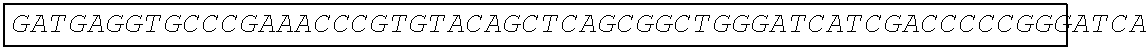 |
| | 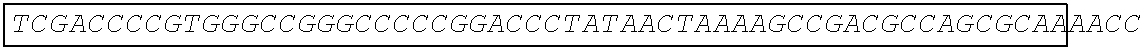 |
| | 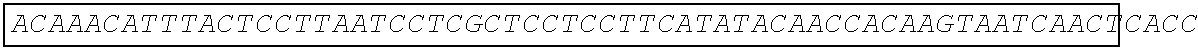 |
| | 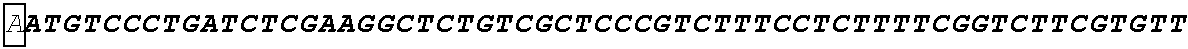 |
| | CTACTGGTCGCTGCCTTTTTTCGCCACCACGTACCGCATCCGCTTCGCCAACGTCGGTA |
| | AGCGAAACGACATGCTGGACTGGGCTAAGACGCTGGTCGCGTTTTTCCGCGTCACCGT |
| | CCTGAAGCAGGGCAACATGTCCCTCTACCGTGGGGGCCGCTGCCTGTACCTGTGCAAC |
| | CACCGCTCCTGGGCCGACTTTTTCATCGACACCTACCTGACGGAGGGCCGCGCGGCCC |
| | TGATGTCCCGGTGGCTCGTGTACTTTGTCTTCCCCGTGTTCTGCACGAGCGTGATTATC |
| | CTCCGAGGGATCGTGCTGTTTAAGCGTGGCCACATCGCGGACAAGGAGGCTTTTAACG |
| | CGTGGCTCGACGCCACGTTCTCCCGCAGCATCGTGCCGGGCCTCCTGGTGTACCCCGA |
| | GGGGCACCGCTCCCTGAAGCGCACCTCTCTGCCCCTGAAGCGGGGTATGATGCACTAC |
| | GCTTACTCGCGCCGAATCCCCGTGCAACTGATCGTGACCCGCGGCAAGGAGGAGGTCC |
| | TGTCGGAGAAGACCCTCTCGGCGCGGTGGGGCCGAACCCTGGTCACGACCTACAGCAA |
| | GGTCCTGAAGCCGTCCGACTACGACTCCTTCGACGCCTTCTTTAACGACATCCAGGCGC |
| | AGTGGGACGCCTGCTGGAAGACGAGCTACGACACCCCAGCCGCGGGCGTCCCGCAGCT |
| | CTCCATGGTCGATGCCCACGAGTACGACTACCCCCGTCGAATGTGGTGGCTGCAACTG |
| | GGCATGACCCTCCTGTCCATCGTGATCCTGGCCGCGGTCCTGTGGGGCTCCTGGCTGG |
| | GCATCCGTGCGGGCCTGGCCACGCTGGGCTCCCTCCAGCAACAGGCCGTCGTGTGGGG |
| | CGCTTCCGGCTGGCTGGGCGCCTCGCTCCTGCTCTCGTTCTTCTGAgtaccacgcccgcgcggcg |
| | cacctgacctgttctctcgagggcgcctgttctgccttgcgaaacaagcccctggagcatgcgtgcatgatcgtctctggegccccgcegcgc |
| | ggtttgtcgccctcgcgggcgccccggccgcgggggcgcattgaaattgttgcaaaccccacctgacagattgagggcccaggcaggaag |
| | gcgttgagatggaggtacaggagtcaagtaactgaaagtttttatgataactaacaacaaagggtcgtttctggccagcgaatgacaagaaca |
| | agattccacatttccgtgtagaggcttgccatcgaatgtgagcggggggccgeggacccgacaaaaccettacgacgtggtaagaaaaac |
| | gtggcgggcactgtccctgtagcctgaagaccagcaggagacgatcggaagcatcacagcacaggccggcccgcgtctcgaacagagcg |
| | cggcggccgcatcgatacctgcaggacagcgccatgccacgccctttgatggcttcaagtacgattacggtgttggattgtgtgtttgttg |
| | cgtagtgtgcatggtttagaataatacacttgatttcttgctcacggcaatctcggcttgtccgcaggttcaaccccatttcggagtctca |
| | ggtcagccgcgcaatgaccagccgctacttcaaggacttgcacgacaacgccgaggtgagctatgtttaggacttgattggaaattgt |
| | cgtcgacgcatattcgcgctccgcgacagcacccaagcaaaatgtcaagtgcgttccgatttgcgtccgcaggtcgatgttgtgatcgt |
| | cggcgccggatccgccggtctgtcctgcgcttacgagctgaccaagcaccctgacgtccgggtacgcgagctgagattcgattagac |
| | ataaattgaagattaaacccgtagaaaaatttgatggtcgcgaaactgtgctcgattgcaagaaattgatcgtcctccactccgcagg |
| | tcgccatcatcgagcagggcgttgctcccggcggcggcgcctggctgggggg ggggggacagctgttctcggccatgtgtgtacgtagaagga |
| | tgaatttcagctggttttcgttgcacagctgtttgtgcatgatttgtttcagactattgttgaatgtttttagatttcttaggatgcatgatttg |
| | tctgcatgcgact |

The construct can be written as 5′Thia4::CrTUB2:ScSUC2:PmPGH:CvNR:PmG3PDH:EcylLPAAT2:PmPGH::3′Thia4. Proceeding in the 5′ to 3′ direction, bold, lowercase sequences represent genomic DNA from CHK22 that permit targeted integration at the Thia4 locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR that enables amplification of the EcylLPAAT2 gene, is indicated by uppercase underlined text followed by a linker, indicated by lowercase, bold italics, which contains a C. vulgaris nitrate reductase 3′-UTR. The P. moriformis PmG3PDH promoter, indicated by boxed italicized text, drive the expression of the EcylLPAAT2. The initiator ATG and terminator TGA codons of the EcylLPAAT2 are indicated by uppercase, bold italics, while the remainder of the gene is indicated by bold italics. The PmPGH 3′-UTR is indicated by lowercase underlined text followed by the CHK22 Thia4 genomic region indicated by bold, lowercase text.

Table 89 shows the fatty acid/TAG profiles of eight primary transformants of CHK22 transformed with pCHK1217. The P. moriformis base strain CHK22 is shown as a non-transgenic control. Strains were grown for 96 hrs in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 89
Screen of primary transformants of CHK22 with pCHK1217.

| | FAME Profiles | | | | | | | | | TAG Profile |
|---|---|---|---|---|---|---|---|---|---|---|
| | | | | | | | C18:3 | | | m/z 577/603 |
| Sample | C12:0 | C14:0 | C16:0 | C18:0 | C18:1 | C18:2 | alpha | C20:0 | C20:1 | (OP:OO) |
| pCHK1217-1 | 0.03 | 1.25 | 27.10 | 2.86 | 60.21 | 6.45 | 0.48 | 0.22 | 0.00 | 1.7 |
| pCHK1217-2 | 0.04 | 1.29 | 26.09 | 2.51 | 59.66 | 8.12 | 0.51 | 0.20 | 0.04 | 1.8 |
| pCHK1217-3 | 0.03 | 1.31 | 27.08 | 3.21 | 60.00 | 6.33 | 0.50 | 0.22 | 0.00 | 1.6 |
| pCHK1217-4 | 0.03 | 1.29 | 27.33 | 3.04 | 59.62 | 6.59 | 0.50 | 0.23 | 0.03 | 1.5 |
| pCHK1217-5 | 0.04 | 1.47 | 27.55 | 2.50 | 58.97 | 7.06 | 0.57 | 0.21 | 0.05 | 1.6 |
| pCHK1217-6 | 0.03 | 1.19 | 26.65 | 2.91 | 60.50 | 6.60 | 0.49 | 0.24 | 0.03 | 1.7 |
| pCHK1217-7 | 0.03 | 1.24 | 26.95 | 3.00 | 60.13 | 6.55 | 0.50 | 0.24 | 0.03 | 1.6 |
| pCHK1217-8 | 0.04 | 1.33 | 27.76 | 2.84 | 57.86 | 7.86 | 0.60 | 0.25 | 0.00 | 2 |
| CHK22 Control | 0.04 | 1.4 | 25.8 | 2.69 | 61.83 | 6.51 | 0.36 | 0.16 | 0 | 1.5 |

### Example 43: Expressing Gonium multicoccum (GmulLPAAT2) in P. moriformis Strain CHK22 Using the PmG3PDH Promoter

GmulLPAAT2 was introduced into a P. moriformis strain CHK22. The expression construct pCHK1218 contained 5′ and 3′ homology arms to permit its targeted integration into the genome and is shown in Table 90.

TABLE 90
Integrative sequences for the transformation of P. moriformis with pCHK1218
encoding GmulLPAAT2.

| SEQ | |
|---|---|
| ID | |
| NO: | Nucleotide Sequence |
| 73 | ccctcaactgcgacgctgggaaccttctccgggcaggcgatgtgcgtgggtttgcctccttggcacggctctacaccgtcgagtacgcc |
| | atgaggcggtgatggctgtgtcggttgccacttcgtccagagacggcaagtcgtccatcctctgcgtgtgtggcgcgacgctgcagca |
| | gtccctctgcagcagatgagcgtgactttggccatttcacgcactegagtgtacacaatccatttttcttaaagcaaatgactgctgatt |
| | gaccagatactgtaacgctgatttcgctccagategcacagatagegaccatgttgctgcgtctgaaaatctggattccgaattegacc |
| | ctggcgctccatccatgcaacagatggcgacacttgttacaattcctgtcacccateggcatggagcaggtccacttagattcccgatc |
| | acccacgcacatctcgctaatagtcattcgttcgtgtcttcgatcaatctcaagtgagtgtgcatggatcttggttgacgatgcggtatg |
| | ggtttgcgccgctggctgcagggtctgcccaaggcaagctaacccagetcctctccccgacaatactctcgcaggcaaagccggtca |
| | cttgccttccagattgccaataaactcaattatggcctctgtcatgccatccatgggtctgatgaatggtcacgctcgtgtcctgaccgtt |
| | 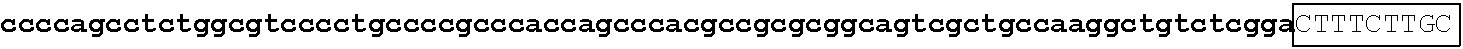 |
| | 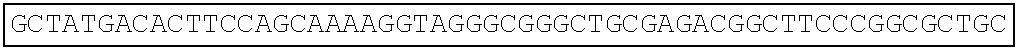 |
| | 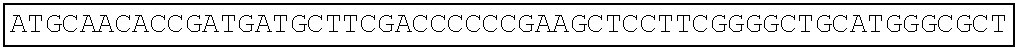 |
| | 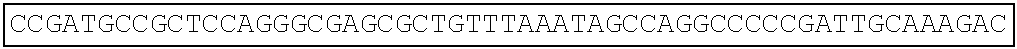 |
| | 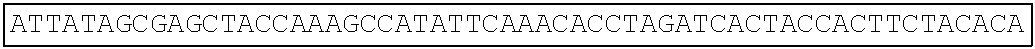 |
| | 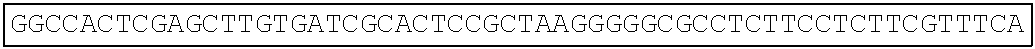 |
| | 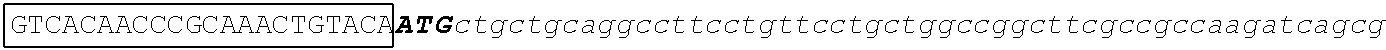 |
| | cctccatgacgaacgagacgtccgaccgccccctggtgcacttcacccccaacaagggctggatgaacgaccccaacggcctgtggta |
| | cgacgagaaggacgccaagtggcacctgtacttccagtacaacccgaacgacaccgtctgggggacgcccttgttctggggccacgcc |
| | acgtccgacgacctgaccaactgggaggaccagcccatcgccatcgccccgaagcgcaacgactccggcgccttctccggctccatgg |
| | tggtggactacaacaacacctccggcttcttcaacgacaccatcgacccgcgccagcgctgcgtggccatctggacctacaacaccccg |
| | gagtccgaggagcagtacatctcctacagcctggacggcggctacaccttcaccgagtaccagaagaaccccgtgctggccgccaact |
| | ccacccagttccgcgacccgaaggtcttctggtacgagccctcccagaagtggatcatgaccgcggccaagtcccaggactacaagat |
| | cgagatctactcctccgacgacctgaagtcctggaagctggagtccgcgttcgccaacgagggcttcctcggctaccagtacgagtgccc |
| | cggcctgatcgaggtccccaccgagcaggaccccagcaagtcctactgggtgatgttcatctccatcaaccccggcgccccggccggc |
| | ggctccttcaaccagtacttcgtcggcagcttcaacggcacccacttcgaggccttcgacaaccagtcccgcgtggtggacttcggcaag |
| | gactactacgccctgcagaccttcttcaacaccgacccgacctacgggagcgccctgggcatcgcgtgggcctccaactgggagtactc |
| | cgccttcgtgcccaccaacccctggcgctcctccatgtccctcgtgcgcaagttctccctcaacaccgagtaccaggccaacccggagac |
| | ggagctgatcaacctgaaggccgagccgatcctgaacatcagcaacgccggcccctggagccggttcgccaccaacaccacgttgac |
| | gaaggccaacagctacaacgtcgacctgtccaacagcaccggcaccctggagttcgagctggtgtacgccgtcaacaccacccagac |
| | gatctccaagtccgtgttcgcggacctctccctctggttcaagggcctggaggaccccgaggagtacctccgcatgggcttcgaggtgtcc |
| | gcgtcctccttcttcctggaccgcgggaacagcaaggtgaagttcgtgaaggagaacccctacttcaccaaccgcatgagcgtgaacaa |
| | ccagcccttcaagagcgagaacgacctgtcctactacaaggtgtacggcttgctggaccagaacatcctggagctgtacttcaacgacg |
| | gcgacgtcgtgtccaccaacacctacttcatgaccaccgggaacgccctgggctccgtgaacatgacgacgggggggacaacctgttc |
| | tacatcgacaagttccaggtgcgcgaggtcaagtgactTAAGACGCCCGCGCGGCGCACCTGACCTGTTCT |
| | CTCGAGGGCGCCTGTTCTGCCTTGCGAAACAAGCCCCTGGAGCATGCGTGCATGATCG |
| | TCTCTGGCGCCCCGCCGCGCGGTTTGTCGCCCTCGCGGGCGCCGCGGCCGCGGGGGC |
| | GCATTGAAATTGTTGCAAACCCCACCTGACAGATTGAGGGCCCAGGCAGGAAGGCGT |
| | TGAGATGGAGGTACAGGAGTCAAGTAACTGAAAGTTTTTATGATAACTAACAACAAA |
| | GGGTCGTTTCTGGCCAGCGAATGACAAGAACAAGATTCCACATTTCCGTGTAGAGGC |
| | TTGCCATCGAATGTGAGCGGGCGGGCCGCGGACCCGACAAAACCCTTACGACGTGGT |
| | AAGAAAAACGTGGCGGGCACTGTCCCTGTAGCCTGAAGACCAGCAGGAGACGATCG |
| | GAAGCATCACAGCACAACCGGTgcagcagcagctcggatagtatcgacacactctggacgctggtcgtgtgatggac |
| | tgttgccgccacacttgctgccttgacctgtgaatatccctgccgcttttatcaaacagcctcagtgtgtttgatcttgtgtgtacgcgcttttgc |
| | gagttgctagctgcttgtgctatttgcgaataccacccccagcatccccttccctcgtttcatategcttgcatcccaaccgcaacttatctacgc |
| | tgtcctgctatccctcagegctgctcctgctcctgctcactgcccctcgcacagccttggtttgggctccgcctgtattctcctggtactgcaacc |
| | 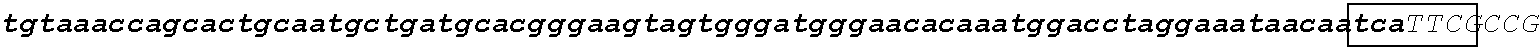 |
| | 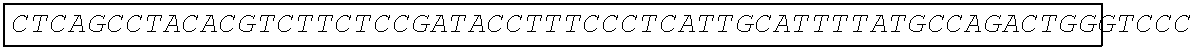 |
| | 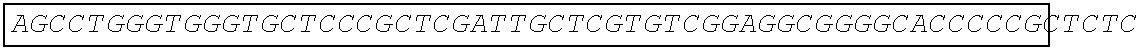 |
| | 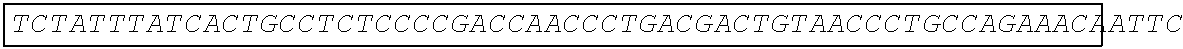 |
| | 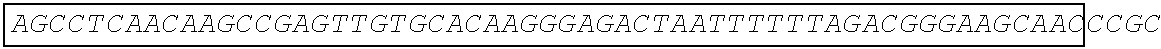 |
| | 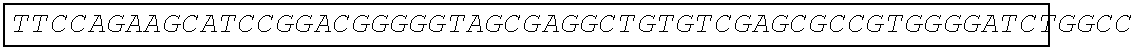 |
| | 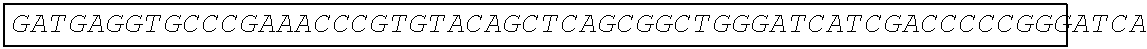 |
| | 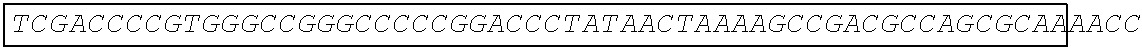 |
| | 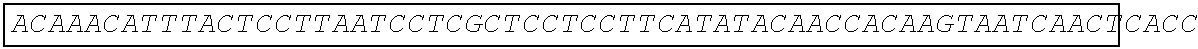 |
| | 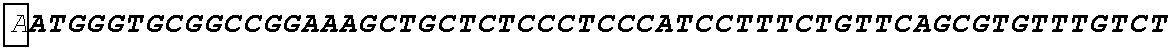 |
| | TTTACTGGAGCCTGCCAATCTTCGCGCTGGTGTACCGTATCCGATTCGCCAGCTCCGGC |
| | CGACGCAACGACATGCTCGACTGGGCTAAGAGCATCGTCCGGTTCTTTGGCGTGACCG |
| | TCGTGAAGGTGGGCGAGAACAGCCTGTACCGCGAGGGGCCATGTCTGTACCTGTGCAA |
| | CCACCGCTCGTGGGCCGATTTTTTCATCGACGCTTACCTCACCGAGGGACGAGCGGCCC |
| | TGATGTCGCGCTGGCTGGTGTTTTTCGTGTTTCCCGTGTTCTGCACCTCCTGCCTGATC |
| | CTGCGCGGCATCGTCCTCTTCAAGCGGGGCACCATCGCCGACAAGGAGGGCTTCAACC |
| | GCTGGCTCGACTCCAAGCTGGGCTCCTCGCACGTGCCTGGTCTCCTGGTCTACCCTGAG |
| | GGCCACCGCAGCCTCCGCCCCTCTTCGCTGCCGCTGAAGCGCGGTATGCTCCACTACG |
| | CCTACGGCCGGAAGCTCGCCGTGCAGATTATCTGCACGGCGGGGAAGGAGGGGGTGAT |
| | GTCGGAGAAGACCATGGCCGTGCACTTCGGCCGGACGCTCGTCACGACCTTCAGCCAG |
| | GTCCTGCGCCCATCTGAGTACCCCACCTTCGACGCCTTTTTCGCGGCCGTGCAGTCCGC |
| | GTGGGATGCGAGCTGGAAGGCCGCGAACCAGACCCCCTCGGCGGGCCTGCCCCGCCTG |
| | TCCATGGAGGGAGCGCAGGACTACGACTACCCGCCCGCGATGCGCGCGCAGCAACTGG |
| | GAGTGACCCTCCTGAGCTTCGTGGTCTTCGGAGGCGCCCTGCTCGTCACGTGGCGACT |
| | CCTGGCTGCCCTCCTGGCGGCTGCGGGTGCGGCCCAACAGGCTGTCGCCCTGCTCCTG |
| | GCGGGCTGGACGGGCGTCTCTATGCTCCGCAGCTTCCTGTGAgtaccacgcccgcgcggcgcacctg |
| | acctgttctctcgagggcgcctgttctgccttgcgaaacaagcccctggagcatgcgtgcatgatcgtctctggcgccccgccgcgcggtttgt |
| | cgccctcgcgggcgccccggccgggggcgcattgaaattgttgcaaaccccacctgacagattgagggcccaggcaggaaggcgttg |
| | agatggaggtacaggagtcaagtaactgaaagtttttatgataactaacaacaaagggtcgtttctggccagegaatgacaagaacaagattcc |
| | acatttccgtgtagaggcttgccatcgaatgtgagcggggggccgcggacccgacaaaacccttacgacgtggtaagaaaaacgtggcgg |
| | gcactgtccctgtagcctgaagaccagcaggagacgatcggaagcatcacagcacaggccggcccgcgtctcgaacagagcgcggcgg |
| | ccgcatcgatacctgcaggacagcgccatgccacgccctttgatggcttcaagtacgattacggtgttggattgtgtgtttgttgcgtagt |
| | gtgcatggtttagaataatacacttgatttcttgctcacggcaatctcggcttgtccgcaggttcaaccccatttcggagtctcaggtcag |
| | ccgcgcaatgaccagccgctacttcaaggacttgcacgacaacgccgaggtgagctatgtttaggacttgattggaaattgtcgtcga |
| | cgcatattegcgctccgcgacagcacccaagcaaaatgtcaagtgcgttccgatttgcgtccgcaggtcgatgttgtgatcgtcggcg |
| | ccggatccgccggtctgtcctgcgcttacgagctgaccaagcaccctgacgtccgggtacgcgagctgagattcgattagacataaat |
| | tgaagattaaacccgtagaaaaatttgatggtcgcgaaactgtgctcgattgcaagaaattgatcgtcctccactccgcaggtcgcca |
| | tcatcgagcagggcgttgctcccggcggcggcgcctggctggggggacagctgttctcggccatgtgtgtacgtagaaggatgaattt |
| | cagctggttttcgttgcacagctgtttgtgcatgatttgtttcagactattgttgaatgtttttagatttcttaggatgcatgatttgtctgcat |
| | gcgact |

The construct can be written as 5′ Thia4::CrTUB2:ScSUC2:PmPGH:CvNR:PmG3PDH:GmulLPAAT2:PmPGH::3′Thia4. Proceeding in the g to 3 direction, bold, lowercase sequences represent genomic DNA from CHK22 that permit targeted integration at the Thia4 locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR that enables amplification of the GmulLPAAT2 gene, is indicated by uppercase underlined text followed by a linker, indicated by lowercase, bold italics, which contains a C. vulgaris nitrate reductase 3′-UTR. The P. moriformis PmG3PDH promoter, indicated by boxed italicized text, drive the expression of the GmulLPAAT2. The initiator ATG and terminator TGA codons of the GmulLPAAT2 are indicated by uppercase, bold italics, while the remainder of the gene is indicated by bold italics. The PmPGH 3′-UTR is indicated by lowercase underlined text followed by the CHK22 Thia4 genomic region indicated by bold, lowercase text.

Table 91 shows the fatty acid/TAG profiles of seven primary transformants of CHK22 transformed with pCHK1218. The P. moriformis base strain CHK22 is shown as a non-transgenic control. Strains were grown for 96 hrs in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 91
Screen of primary transformants of CHK22 with pCHK1218.

| | FAME Profiles | | | | | | | | | TAG Profile |
|---|---|---|---|---|---|---|---|---|---|---|
| | | | | | | | C18:3 | | | m/z 577/603 |
| Sample | C12:0 | C14:0 | C16:0 | C18:0 | C18:1 | C18:2 | alpha | C20:0 | C20:1 | (OP:OO) |
| pCHK1218-1 | 0.04 | 1.51 | 28.72 | 3.37 | 52.27 | 11.15 | 1.14 | 0.29 | 0.00 | 3.9 |
| pCHK1218-2 | 0.03 | 1.29 | 26.95 | 3.30 | 59.47 | 6.85 | 0.50 | 0.27 | 0.03 | 1.6 |
| pCHK1218-3 | 0.03 | 1.32 | 27.80 | 2.83 | 57.95 | 7.92 | 0.51 | 0.22 | 0.00 | 3.7 |
| pCHK1218-4 | 0.03 | 1.33 | 27.71 | 2.75 | 57.81 | 8.21 | 0.51 | 0.21 | 0.00 | 4.2 |
| pCHK1218-5 | 0.03 | 1.33 | 27.58 | 3.13 | 57.84 | 7.97 | 0.52 | 0.24 | 0.00 | 3.8 |
| pCHK1218-6 | 0.04 | 1.59 | 28.47 | 2.82 | 56.81 | 8.40 | 0.33 | 0.19 | 0.00 | 3.5 |
| pCHK1218-7 | 0.04 | 1.37 | 28.24 | 2.83 | 57.34 | 8.03 | 0.52 | 0.21 | 0.00 | 3.9 |
| CHK22 Control | 0.04 | 1.4 | 25.8 | 2.69 | 61.83 | 6.51 | 0.36 | 0.16 | 0 | 1.5 |

### Example 44: Expressing Gonium viridistellatum (GvirLPAAT2) in P. moriformis Strain CHK22 Using the PmG3PDH Promoter

GvirLPAAT2 was introduced into a P. moriformis strain CHK22. The expression construct pCHK1219 contained 5′ and 3′ homology arms to permit its targeted integration into the genome and is shown in Table 92.

TABLE 92
Integrative sequences for the transformation of P. moriformis with pCHK1219
encoding GvirLPAAT2.

| SEQ | |
|---|---|
| ID | |
| NO: | Nucleotide Sequence |
| 74 | ccctcaactgcgacgctgggaaccttctccgggcaggcgatgtgcgtgggtttgcctccttggcacggctctacaccgtcgagtacgcc |
| | atgaggcggtgatggctgtgtcggttgccacttcgtccagagacggcaagtcgtccatcctctgcgtgtgtggcgcgacgctgcagca |
| | gtccctctgcagcagatgagcgtgactttggccatttcacgcactegagtgtacacaatccatttttcttaaagcaaatgactgctgatt |
| | gaccagatactgtaacgctgatttcgctccagatcgcacagatagegaccatgttgctgcgtctgaaaatctggattccgaattegacc |
| | ctggcgctccatccatgcaacagatggcgacacttgttacaattcctgtcacccatcggcatggagcaggtccacttagattcccgatc |
| | acccacgcacatctcgctaatagtcattcgttcgtgtcttcgatcaatctcaagtgagtgtgcatggatcttggttgacgatgcggtatg |
| | ggtttgcgccgctggctgcagggtctgcccaaggcaagctaacccagctcctctccccgacaatactctcgcaggcaaagccggtca |
| | cttgccttccagattgccaataaactcaattatggcctctgtcatgccatccatgggtctgatgaatggtcacgctcgtgtcctgaccgtt |
| | 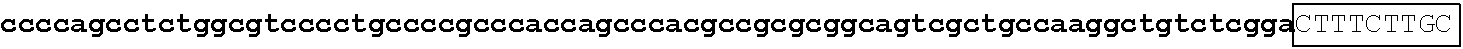 |
| | 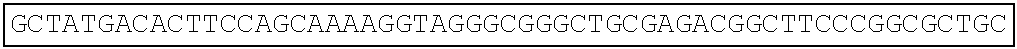 |
| | 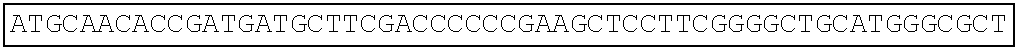 |
| | 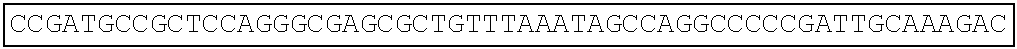 |
| | 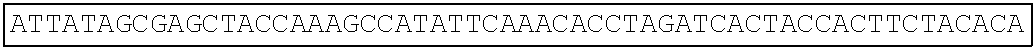 |
| | 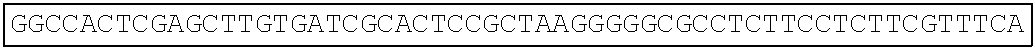 |
| | 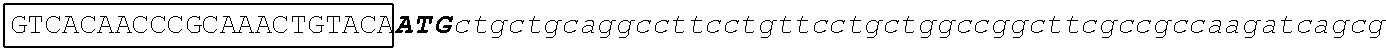 |
| | cctccatgacgaacgagacgtccgaccgccccctggtgcacttcacccccaacaagggctggatgaacgaccccaacggcctgtggta |
| | cgacgagaaggacgccaagtggcacctgtacttccagtacaacccgaacgacaccgtctgggggacgcccttgttctggggccacgcc |
| | acgtccgacgacctgaccaactgggaggaccagcccatcgccatcgccccgaagcgcaacgactccggcgccttctccggctccatgg |
| | tggtggactacaacaacacctccggcttcttcaacgacaccatcgacccgcgccagcgctgcgtggccatctggacctacaacaccccg |
| | gagtccgaggagcagtacatctcctacagcctggacggcggctacaccttcaccgagtaccagaagaaccccgtgctggccgccaact |
| | ccacccagttccgcgacccgaaggtcttctggtacgagccctcccagaagtggatcatgaccgcggccaagtcccaggactacaagat |
| | cgagatctactcctccgacgacctgaagtcctggaagctggagtccgcgttcgccaacgagggcttcctcggctaccagtacgagtgccc |
| | cggcctgatcgaggtccccaccgagcaggaccccagcaagtcctactgggtgatgttcatctccatcaaccccggcgccccggccggc |
| | ggctccttcaaccagtacttcgtcggcagcttcaacggcacccacttcgaggccttcgacaaccagtcccgcgtggtggacttcggcaag |
| | gactactacgccctgcagaccttcttcaacaccgacccgacctacgggagcgccctgggcatcgcgtgggcctccaactgggagtactc |
| | cgccttcgtgcccaccaacccctggcgctcctccatgtccctcgtgcgcaagttctccctcaacaccgagtaccaggccaacccggagac |
| | ggagctgatcaacctgaaggccgagccgatcctgaacatcagcaacgccggcccctggagccggttcgccaccaacaccacgttgac |
| | gaaggccaacagctacaacgtcgacctgtccaacagcaccggcaccctggagttcgagctggtgtacgccgtcaacaccacccagac |
| | gatctccaagtccgtgttcgcggacctctccctctggttcaagggcctggaggaccccgaggagtacctccgcatgggcttcgaggtgtcc |
| | gcgtcctccttcttcctggaccgcgggaacagcaaggtgaagttcgtgaaggagaacccctacttcaccaaccgcatgagcgtgaacaa |
| | ccagcccttcaagagcgagaacgacctgtcctactacaaggtgtacggcttgctggaccagaacatcctggagctgtacttcaacgacg |
| | gcgacgtcgtgtccaccaacacctacttcatgaccaccgggaacgccctgggctccgtgaacatgacgacgggggtggacaacctgttc |
| | tacatcgacaagttccaggtgcgcgaggtcaagtgactTAAGACGCCCGCGCGGCGCACCTGACCTGTTCT |
| | CTCGAGGGCGCCTGTTCTGCCTTGCGAAACAAGCCCCTGGAGCATGCGTGCATGATCG |
| | TCTCTGGCGCCCCGCCGCGCGGTTTGTCGCCCTCGCGGGCGCCGCGGCCGCGGGGGC |
| | GCATTGAAATTGTTGCAAACCCCACCTGACAGATTGAGGGCCCAGGCAGGAAGGCGT |
| | TGAGATGGAGGTACAGGAGTCAAGTAACTGAAAGTTTTTATGATAACTAACAACAAA |
| | GGGTCGTTTCTGGCCAGCGAATGACAAGAACAAGATTCCACATTTCCGTGTAGAGGC |
| | TTGCCATCGAATGTGAGCGGGCGGGCCGCGGACCCGACAAAACCCTTACGACGTGGT |
| | AAGAAAAACGTGGCGGGCACTGTCCCTGTAGCCTGAAGACCAGCAGGAGACGATCG |
| | GAAGCATCACAGCACAACCGGTgcagcagcagctcggatagtatcgacacactctggacgctggtcgtgtgatggac |
| | tgttgccgccacacttgctgccttgacctgtgaatatccctgccgcttttatcaaacagcctcagtgtgtttgatcttgtgtgtacgcgcttttgc |
| | gagttgctagctgcttgtgctatttgcgaataccacccccagcatccccttccctegtttcatategcttgcatcccaaccgcaacttatctacgc |
| | tgtcctgctatccctcagegctgctcctgctcctgctcactgcccctcgcacagccttggtttgggctccgcctgtattctcctggtactgcaacc |
| | 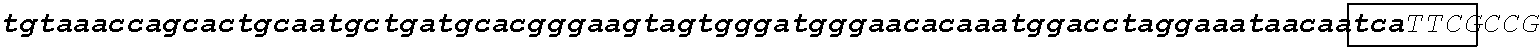 |
| | 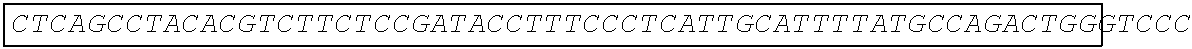 |
| | 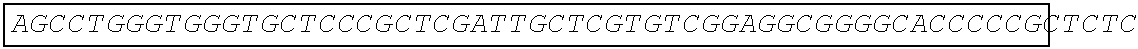 |
| | 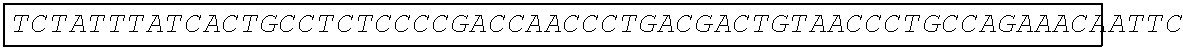 |
| | 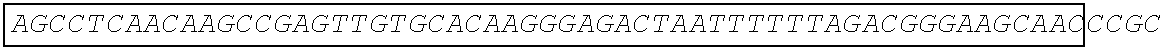 |
| | 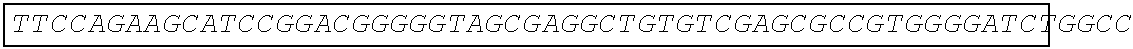 |
| | 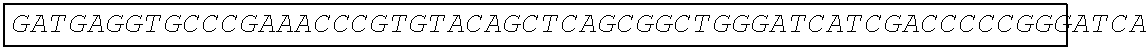 |
| | 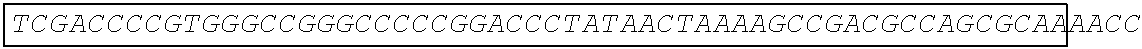 |
| | 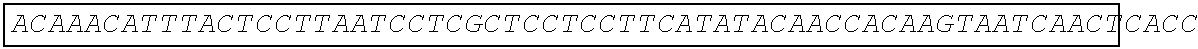 |
| | 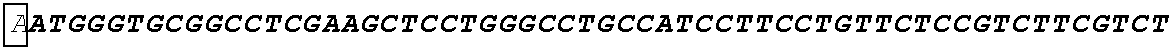 |
| | TTTACTGGTCCCTGCCCATCTTCGCCATCGTCTACCGCATCCGCTTCGCTTCCCCAGGG |
| | CGGCGCAACGACATGCTGGACTGGGCCAAGTCGATTGTGGGTTTTTTCGGCGTGGCCG |
| | TCCACAAGGCGGGTACGAACAGCCTGTACCGAGACGGCCCGTGCCTGTACCTCTGTAA |
| | CCACCGCTCTTGGGCGGACTTTTTCATCGACGCCTACCTCACGGAGGGCCGTGCGGCTC |
| | TGATGTCGCGCTGGCTGGTCTTTTTCGTGTTCCCCGTGTTCTGCACGAGCTGCCTGATC |
| | CTCCGCGGTATCGTCCTCTTCAAGCGCGGCACCATCGCGGACAAGGAGGCCTTTAATAG |
| | CTGGCTGGACGCTACGCTCGGCCGCTCCATCGTGCCCGGTCTCCTGGTCTACCCCGAG |
| | GGCCACCGTTCCCTGCGCCCCGCCTCCCTGCCGCTGAAGCGCGGCATGCTGCACTACG |
| | CTTTTGGTCGCAAGCTGCCCGTGCAGGTGGTCTGCACCGCTGGCAAGGAGGGAGTCAT |
| | GTCGGAGAAGACCATGGCTGTGCGCTTCGGCCGTCGCCTCGTCACCAACTTCTCCCAG |
| | GTGATCCGCCCTGCCGACTTCCCAACGTTCGACGCCTTCAGCGCGGCCGTCCAGTCCGC |
| | CTGGGACGGCTGCTGGAAGGCGGCCTACGAGACCCCCGCTGCGGGCCTCCCTCGCCTG |
| | TCCATGGAGGGCTCGCATGAGTACGAGTACGACTACCCCGCCCCCATGCGCGTCCTCC |
| | AAGTCTGCATCACGCTCCTGTCCATCGTCCTCTTCCTGGGCGTCCTGCTCGGCTCCTGG |
| | CGCGCGCTGGCCGCTGCCCTGGCTGCCGCGGGCGCGGCTCAGCAAGTCGTGGTCGCCC |
| | TCCTGGCGACGTGGCTGGGCGCGTCCATGCTCCGCTCTTGCCTCTGAgtaccacgcccgcgcgg |
| | cgcacctgacctgttctctcgagggcgcctgttctgccttgcgaaacaagcccctggagcatgcgtgcatgatcgtctctggegccccgccgc |
| | gcggtttgtcgccctcgcgggcgccccggccgcgggggcgcattgaaattgttgcaaaccccacctgacagattgagggcccaggcagga |
| | aggcgttgagatggaggtacaggagtcaagtaactgaaagtttttatgataactaacaacaaagggtcgtttctggccagcgaatgacaagaa |
| | caagattccacatttccgtgtagaggcttgccatcgaatgtgagcggggggccgeggacccgacaaaaccettacgacgtggtaagaaaa |
| | acgtggcgggcactgtccctgtagcctgaagaccagcaggagacgatcggaagcatcacagcacaggccggcccgcgtctcgaacagag |
| | cgcggcggccgcatcgatacctgcaggacagcgccatgccacgccctttgatggcttcaagtacgattacggtgttggattgtgtgtttg |
| | ttgcgtagtgtgcatggtttagaataatacacttgatttcttgctcacggcaatctcggcttgtccgcaggttcaaccccatttcggagtct |
| | caggtcagccgcgcaatgaccagccgctacttcaaggacttgcacgacaacgccgaggtgagctatgtttaggacttgattggaaat |
| | tgtcgtcgacgcatattcgcgctccgcgacagcacccaagcaaaatgtcaagtgcgttccgatttgcgtccgcaggtcgatgttgtgat |
| | cgtcggcgccggatccgccggtctgtcctgcgcttacgagctgaccaagcaccctgacgtccgggtacgcgagctgagattcgattag |
| | acataaattgaagattaaacccgtagaaaaatttgatggtcgcgaaactgtgctcgattgcaagaaattgatcgtcctccactccgca |
| | ggtcgccatcatcgagcagggcgttgctcccggcggcggcgcctggctggggggacagctgttctcggccatgtgtgtacgtagaag |
| | gatgaatttcagctggttttcgttgcacagctgtttgtgcatgatttgtttcagactattgttgaatgtttttagatttcttaggatgcatgat |
| | ttgtctgcatgcgact |

The construct can be written as 5′Thia4::CrTUB2:ScSUC2:PmPGH:CvNR:PmG3PDH:GvirLPAAT2:PmPGH::3′Thia4. Proceeding in the 5′ to 3′ direction, bold, lowercase sequences represent genomic DNA from CHK22 that permit targeted integration at the Thia4 locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR that enables amplification of the GvirLPAAT2 gene, is indicated by uppercase underlined text followed by a linker, indicated by lowercase, bold italics, which contains a C. vulgaris nitrate reductase 3′-UTR. The P. moriformis PmG3PDH promoter, indicated by boxed italicized text, drive the expression of the GvirLPAAT2. The initiator ATG and terminator TGA codons of the GvirLPAAT2 are indicated by uppercase, bold italics, while the remainder of the gene is indicated by bold italics. The PmPGH 3′-UTR is indicated by lowercase underlined text followed by the CHK22 Thia4 genomic region indicated by bold, lowercase text.

Table 93 shows the fatty acid/TAG profiles of seven primary transformants of CHK22 transformed with pCHK1219. The P. moriformis base strain CHK22 is shown as a non-transgenic control. Strains were grown for 120 hrs in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 93
Screen of primary transformants of CHK22 with pCHK1219.

| | FAME Profiles | | | | | | | | | TAG Profile |
|---|---|---|---|---|---|---|---|---|---|---|
| | | | | | | | C18:3 | | | m/z 577/603 |
| Sample | C12:0 | C14:0 | C16:0 | C18:0 | C18:1 | C18:2 | alpha | C20:0 | C20:1 | (OP:OO) |
| pCHK1219-1 | 0.04 | 1.47 | 28.49 | 3.05 | 57.19 | 7.66 | 0.53 | 0.22 | 0.00 | 3.5 |
| pCHK1219-2 | 0.04 | 1.40 | 28.61 | 2.69 | 57.45 | 7.61 | 0.53 | 0.21 | 0.00 | 3.6 |
| pCHK1219-3 | 0.04 | 1.40 | 28.80 | 2.77 | 57.34 | 7.49 | 0.52 | 0.21 | 0.00 | 3.6 |
| pCHK1219-4 | 0.06 | 2.05 | 29.69 | 2.95 | 54.85 | 8.02 | 0.73 | 0.21 | 0.00 | 3.2 |
| pCHK1219-5 | 0.04 | 1.45 | 28.74 | 2.88 | 56.88 | 7.84 | 0.53 | 0.22 | 0.00 | 3.4 |
| pCHK1219-6 | 0.04 | 1.42 | 29.01 | 3.19 | 57.37 | 6.86 | 0.47 | 0.26 | 0.00 | 2.3 |
| pCHK1219-7 | 0.04 | 1.34 | 27.57 | 2.54 | 58.15 | 8.13 | 0.54 | 0.20 | 0.00 | 3.6 |
| pCHK1219-8 | 0.04 | 1.49 | 28.95 | 3.11 | 56.65 | 7.65 | 0.53 | 0.23 | 0.00 | 3.3 |
| CHK22 Control | 0.04 | 1.4 | 25.8 | 2.69 | 61.83 | 6.51 | 0.36 | 0.16 | 0 | 1.5 |

### Example 45: Expressing Volvox ferrisii (VferLPAAT2) in P. moriformis Strain CHK22 Using the PmG3PDH Promoter

VferLPAAT2 was introduced into a P. moriformis strain CHK22. The expression construct pCHK1220 contained 5′ and 3′ homology arms to permit its targeted integration into the genome and is shown in Table 94.

TABLE 94
Integrative sequences for the transformation of P. moriformis with pCHK1220
encoding VferLPAAT2.

| SEQ | |
|---|---|
| ID | |
| NO: | Nucleotide Sequence |
| 75 | ccctcaactgcgacgctgggaaccttctccgggcaggcgatgtgcgtgggtttgcctccttggcacggctctacaccgtcgagtacgcc |
| | atgaggcggtgatggctgtgtcggttgccacttcgtccagagacggcaagtegtccatcctctgcgtgtgtggcgcgacgctgcagca |
| | gtccctctgcagcagatgagcgtgactttggccatttcacgcactegagtgtacacaatccatttttcttaaagcaaatgactgctgatt |
| | gaccagatactgtaacgctgatttcgctccagatcgcacagatagcgaccatgttgctgcgtctgaaaatctggattccgaattcgacc |
| | ctggcgctccatccatgcaacagatggcgacacttgttacaattcctgtcacccatcggcatggagcaggtccacttagattccegatc |
| | acccacgcacatctcgctaatagtcattcgttcgtgtcttcgatcaatctcaagtgagtgtgcatggatcttggttgacgatgcggtatg |
| | ggtttgcgccgctggctgcagggtctgcccaaggcaagctaacccagetcetctccccgacaatactctcgcaggcaaagccggtca |
| | cttgccttccagattgccaataaactcaattatggcctctgtcatgccatccatgggtctgatgaatggtcacgctcgtgtcctgaccgtt |
| | 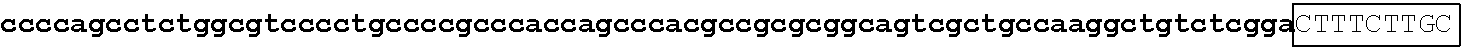 |
| | 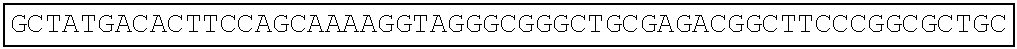 |
| | 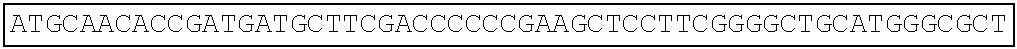 |
| | 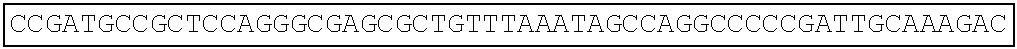 |
| | 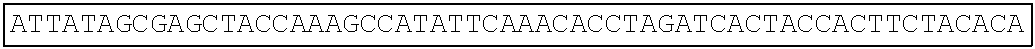 |
| | 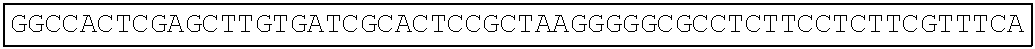 |
| | 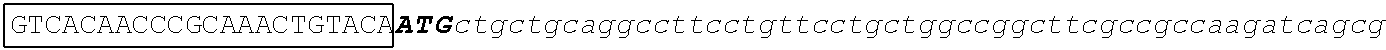 |
| | cctccatgacgaacgagacgtccgaccgccccctggtgcacttcacccccaacaagggctggatgaacgaccccaacggcctgtggta |
| | cgacgagaaggacgccaagtggcacctgtacttccagtacaacccgaacgacaccgtctgggggacgcccttgttctggggccacgcc |
| | acgtccgacgacctgaccaactgggaggaccagcccatcgccatcgccccgaagcgcaacgactccggcgccttctccggctccatgg |
| | tggtggactacaacaacacctccggcttcttcaacgacaccatcgacccgcgccagcgctgcgtggccatctggacctacaacaccccg |
| | gagtccgaggagcagtacatctcctacagcctggacggcggctacaccttcaccgagtaccagaagaaccccgtgctggccgccaact |
| | ccacccagttccgcgacccgaaggtcttctggtacgagccctcccagaagtggatcatgaccgcggccaagtcccaggactacaagat |
| | cgagatctactcctccgacgacctgaagtcctggaagctggagtccgcgttcgccaacgagggcttcctcggctaccagtacgagtgccc |
| | cggcctgatcgaggtccccaccgagcaggaccccagcaagtcctactgggtgatgttcatctccatcaaccccggcgccccggccggc |
| | ggctccttcaaccagtacttcgtcggcagcttcaacggcacccacttcgaggccttcgacaaccagtcccgcgtggtggacttcggcaag |
| | gactactacgccctgcagaccttcttcaacaccgacccgacctacgggagcgccctgggcatcgcgtgggcctccaactgggagtactc |
| | cgccttcgtgcccaccaacccctggcgctcctccatgtccctcgtgcgcaagttctccctcaacaccgagtaccaggccaacccggagac |
| | ggagctgatcaacctgaaggccgagccgatcctgaacatcagcaacgccggcccctggagccggttcgccaccaacaccacgttgac |
| | gaaggccaacagctacaacgtcgacctgtccaacagcaccggcaccctggagttcgagctggtgtacgccgtcaacaccacccagac |
| | gatctccaagtccgtgttcgcggacctctccctctggttcaagggcctggaggaccccgaggagtacctccgcatgggcttcgaggtgtcc |
| | gcgtcctccttcttcctggaccgcgggaacagcaaggtgaagttcgtgaaggagaacccctacttcaccaaccgcatgagcgtgaacaa |
| | ccagcccttcaagagcgagaacgacctgtcctactacaaggtgtacggcttgctggaccagaacatcctggagctgtacttcaacgacg |
| | gcgacgtcgtgtccaccaacacctacttcatgaccaccgggaacgccctgggctccgtgaacatgacgacgggggtggacaacctgttc |
| | tacatcgacaagttccaggtgcgcgaggtcaagtgactTAAGACGCCCGCGCGGCGCACCTGACCTGTTCT |
| | CTCGAGGGCGCCTGTTCTGCCTTGCGAAACAAGCCCCTGGAGCATGCGTGCATGATCG |
| | TCTCTGGCGCCCCGCCGCGCGGTTTGTCGCCCTCGCGGGCGCCGCGGCCGCGGGGGC |
| | GCATTGAAATTGTTGCAAACCCCACCTGACAGATTGAGGGCCCAGGCAGGAAGGCGT |
| | TGAGATGGAGGTACAGGAGTCAAGTAACTGAAAGTTTTTATGATAACTAACAACAAA |
| | GGGTCGTTTCTGGCCAGCGAATGACAAGAACAAGATTCCACATTTCCGTGTAGAGGC |
| | TTGCCATCGAATGTGAGCGGGCGGGCCGCGGACCCGACAAAACCCTTACGACGTGGT |
| | AAGAAAAACGTGGCGGGCACTGTCCCTGTAGCCTGAAGACCAGCAGGAGACGATCG |
| | GAAGCATCACAGCACAACCGGTgcagcagcagctcggatagtatcgacacactctggacgctggtcgtgtgatggac |
| | tgttgccgccacacttgctgccttgacctgtgaatatccctgccgcttttatcaaacagcctcagtgtgtttgatcttgtgtgtacgcgcttttgc |
| | gagttgctagctgcttgtgctatttgcgaataccacccccagcatccccttccctegtttcatategcttgcatcccaaccgcaacttatctacgc |
| | tgtcctgctatccctcagegctgctcctgctcctgctcactgcccctcgcacagccttggtttgggctccgcctgtattctcctggtactgcaacc |
| | 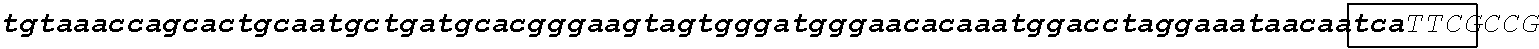 |
| | 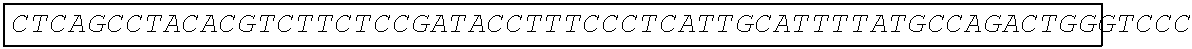 |
| | 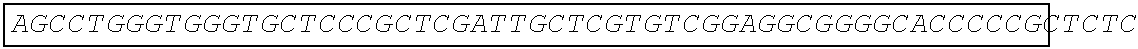 |
| | 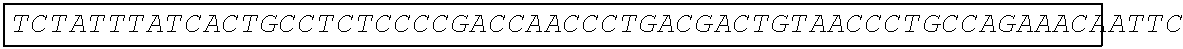 |
| | 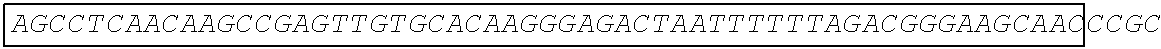 |
| | 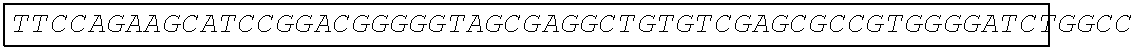 |
| | 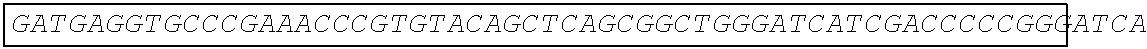 |
| | 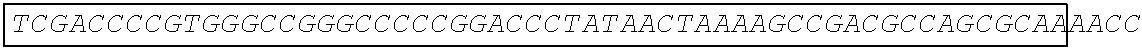 |
| | 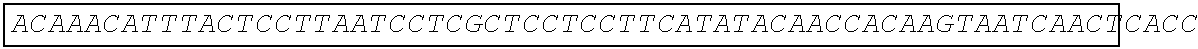 |
| | 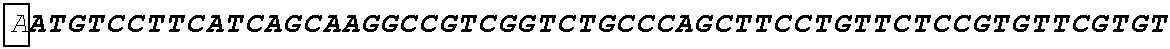 |
| | TCTACTGGAGCCTGCCCATCTTCGCGCTGACGTACCGCATCCGCTTCGCGTCGAAGGGC |
| | AAGCGCAACGACATGCTGGAGTGGGCTAAGGCCCTGGTCCGCTTTTTCGGCGTGAAGG |
| | TGCTCACGCAGGGCGAGCACTCGCTGTACCGCGGGGGCCCGTGCCTCTACCTCTGCAA |
| | CCATCGGAGCTGGGCCGACTTTTTCATCGACGCCTACCTGACCGAGGGCCGAGCTGCG |
| | CTGATGAGCCGATGGCTCGTCTACTTCGTGTTCCCCATCTTCTGTACCTCCGTGATGGT |
| | CCTGCGCGGGATCGTCCTCTTCAAGCGTGGGACCATCGCCGATAAGGAGGCCTTCAAC |
| | GCCTGGCTCGACGCCAAGATGAAGTCCTCGACCATTCCGGGGCTCCTGGTCTACCCCG |
| | AGGGGCACCGCAGCCTCAAGCGCACGTCTCTGCCCCTCAAGCGGGGCATGCTCCATTT |
| | TGCGTTCTCGCGCGGCCTGCCCGTCCAACTCGTCATGTGTGCTGGCAAGGAGGAGGTC |
| | CTGTCTGAGAAGCGCATGGCCGTGCGCTTCGGTCGGACCCTCGTCGCCACGTACTCCG |
| | AGGTCCTCAAGCCCGGCAACTACCCGACGTTCGACGCCTTCTTTTCCGGCATCCAGACG |
| | GCCTGGGATAGCTGCTGGCAGGCGGCTTACAAGCAGCCTCCCGAGGGTGCCGCGCCCC |
| | TGCCCATGGTGAACACGACCGAGTACGACTATCCCCCGCGCATGCGCCGTCTGCAACT |
| | GATCGTGACCCTCCTGTCGATCGTCGTGCTGGCCGCGGTGCTCGCGGGCAGCGTCCTG |
| | GTCACCGTGGTCGCCGCTGCCGCGGTGGGCCCCATGCCAGCGGGCGTGGTCGTGGCCG |
| | TGCTCGCTAGCTGGCTCGGGGGCAGCATGCTGCGTTCTTTCCTCTGAgtaccacgcccgcgcggc |
| | gcacctgacctgttctctcgagggcgcctgttctgccttgcgaaacaagcccctggagcatgcgtgcatgatcgtctctggcgccccgccgcg |
| | cggtttgtcgccctcgcgggcgccccggccgcgggggcgcattgaaattgttgcaaaccccacctgacagattgagggcccaggcaggaa |
| | ggcgttgagatggaggtacaggagtcaagtaactgaaagtttttatgataactaacaacaaagggtcgtttctggccagcgaatgacaagaac |
| | aagattccacatttccgtgtagaggcttgccatcgaatgtgagcggggcgggccgcggacccgacaaaacccttacgacgtggtaagaaaaa |
| | cgtggcgggcactgtccctgtagcctgaagaccagcaggagacgatoggaagcatcacagcacaggccggcccgcgtctogaacagagc |
| | gcggcggccgcatcgatacctgcaggacagcgccatgccacgccctttgatggcttcaagtacgattacggtgttggattgtgtgtttgtt |
| | gcgtagtgtgcatggtttagaataatacacttgatttcttgctcacggcaatctcggcttgtccgcaggttcaaccccattteggagtctc |
| | aggtcagccgcgcaatgaccagccgctacttcaaggacttgcacgacaacgccgaggtgagctatgtttaggacttgattggaaatt |
| | gtcgtcgacgcatattegcgctccgcgacagcacccaagcaaaatgtcaagtgcgttccgatttgcgtccgcaggtcgatgttgtgatc |
| | gtcggcgccggatccgccggtctgtcctgcgcttacgagctgaccaagcaccctgacgtccgggtacgcgagctgagattcgattaga |
| | cataaattgaagattaaacccgtagaaaaatttgatggtcgcgaaactgtgctcgattgcaagaaattgatcgtcctccactccgcag |
| | gtcgccatcatcgagcagggcgttgctcccggcggcggcgcctggctggggggacagctgttctcggccatgtgtgtacgtagaagg |
| | atgaatttcagctggttttcgttgcacagctgtttgtgcatgatttgtttcagactattgttgaatgtttttagatttcttaggatgcatgattt |
| | gtctgcatgcgact |

The construct can be written as 5′Thia4::CrTUB2:ScSUC2:PmPGH:CvNR:PmG3PDH:VferLPAAT2:PmPGH::3′Thia4. Proceeding in the 5′ to 3′ direction, bold, lowercase sequences represent genomic DNA from CHK22 that permit targeted integration at the Thia4 locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR that enables amplification of the VferLPAAT2 gene, is indicated by uppercase underlined text followed by a linker, indicated by lowercase, bold italics, which contains a C. vulgaris nitrate reductase 3′-UTR. The P. moriformis PmG3PDH promoter, indicated by boxed italicized text, drive the expression of the VferLPAAT2. The initiator ATG and terminator TGA codons of the VferLPAAT2 are indicated by uppercase, bold italics, while the remainder of the gene is indicated by bold italics. The PmPGH 3′-UTR is indicated by lowercase underlined text followed by the CHK22 Thia4 genomic region indicated by bold, lowercase text.

Table 95 shows the fatty acid/TAG profiles of eight primary transformants of CHK22 transformed with pCHK1220. The P. moriformis base strain CHK22 is shown as a non-transgenic control. Strains were grown for 120 hrs in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 95
Screen of primary transformants of CHK22 with pCHK1220.

| | FAME Profiles | | | | | | | | | TAG Profile |
|---|---|---|---|---|---|---|---|---|---|---|
| | | | | | | | C18:3 | | | m/z 577/603 |
| Sample | C12:0 | C14:0 | C16:0 | C18:0 | C18:1 | C18:2 | alpha | C20:0 | C20:1 | (OP:OO) |
| pCHK1220-1 | 0.03 | 1.20 | 27.20 | 2.94 | 59.80 | 6.56 | 0.49 | 0.27 | 0.00 | 1.57 |
| pCHK1220-2 | 0.03 | 1.20 | 26.82 | 3.00 | 60.34 | 6.37 | 0.47 | 0.27 | 0.00 | 1.59 |
| pCHK1220-3 | 0.03 | 1.20 | 26.72 | 2.99 | 60.48 | 6.36 | 0.47 | 0.26 | 0.00 | 1.51 |
| pCHK1220-4 | 0.03 | 1.29 | 27.67 | 2.76 | 58.35 | 7.65 | 0.48 | 0.24 | 0.00 | 3.69 |
| pCHK1220-5 | 0.03 | 1.21 | 27.13 | 2.98 | 59.91 | 6.48 | 0.48 | 0.27 | 0.00 | 1.48 |
| pCHK1220-6 | 0.04 | 1.29 | 27.77 | 2.76 | 58.46 | 7.42 | 0.48 | 0.24 | 0.00 | 3.3 |
| pCHK1220-7 | 0.03 | 1.24 | 27.68 | 2.58 | 58.86 | 7.34 | 0.48 | 0.23 | 0.00 | 3.8 |
| pCHK1220-8 | 0.03 | 1.20 | 27.20 | 2.97 | 59.80 | 6.52 | 0.49 | 0.27 | 0.00 | 1.49 |
| CHK22 Control | 0.04 | 1.27 | 27.29 | 2.45 | 59.08 | 7.45 | 0.54 | 0.23 | 0.00 | 1.5 |

### Example 46: Expressing Vitreochlamys aulata (VaulLPAAT2) in P. moriformis Strain CHK22 Using the PmG3PDH Promoter

VaulLPAAT2 was introduced into a P. moriformis strain CHK22. The expression construct pCHK1222 contained 5′ and 3′ homology arms to permit its targeted integration into the genome and is shown in Table 96.

TABLE 96
Integrative sequences for the transformation of P. moriformis with pCHK1222
encoding VaulLPAAT2.

| SEQ ID | |
|---|---|
| NO: | Nucleotide Sequence |
| 76 | ccctcaactgcgacgctgggaaccttctccgggcaggcgatgtgcgtgggtttgcctccttggcacggctctacaccgtcga |
| | gtacgccatgaggcggtgatggctgtgtcggttgccacttcgtccagagacggcaagtcgtccatcctctgcgtgtgtggcg |
| | cgacgctgcagcagtccctctgcagcagatgagcgtgactttggccatttcacgcactcgagtgtacacaatccatttttct |
| | taaagcaaatgactgctgattgaccagatactgtaacgctgatttcgctccagatcgcacagatagcgaccatgttgctgcg |
| | tctgaaaatctggattccgaattcgaccctggcgctccatccatgcaacagatggcgacacttgttacaattcctgtcaccc |
| | atcggcatggagcaggtccacttagattcccgatcacccacgcacatctcgctaatagtcattcgttcgtgtcttcgatcaa |
| | tctcaagtgagtgtgcatggatcttggttgacgatgcggtatgggtttgcgccgctggctgcagggtctgcccaaggcaagc |
| | taacccagctcctctccccgacaatactctcgcaggcaaagccggtcacttgccttccagattgccaataaactcaattatg |
| | gcctctgtcatgccatccatgggtctgatgaatggtcacgctcgtgtcctgaccgtt |
| | 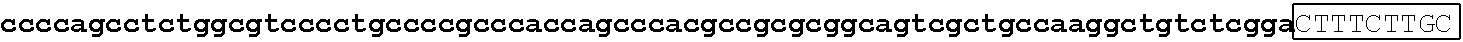 |
| | 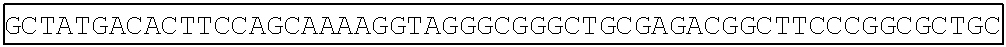 |
| | 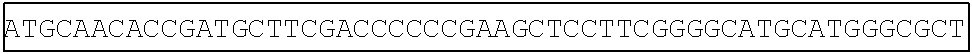 |
| | 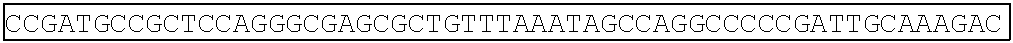 |
| | 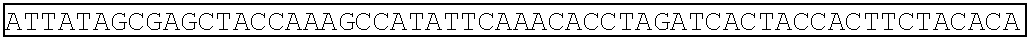 |
| | 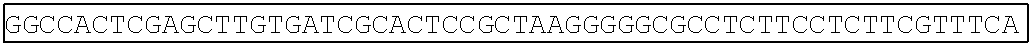 |
| | 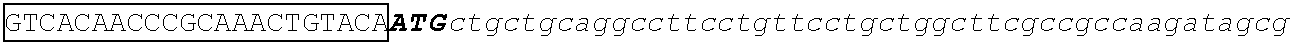 |
| | cctccatgacgaacgagacgtccgaccgccccctggtgcacttcacccccaacaagggctggatgaacgaccccaacggcct |
| | gtggtacgacgagaaggacgccaagtggcacctgtacttccagtacaacccgaacgacaccgtctgggggacgcccttgttc |
| | tggggccacgccacgtccgacgacctgaccaactgggaggaccagcccatcgccatcgccccgaagcgcaacgactccggcg |
| | ccttctccggctccatggtggtggactacaacaacacctccggcttcttcaacgacaccatcgacccgcgccagcgctgcgt |
| | ggccatctggacctacaacaccccggagtccgaggagcagtacatctcctacagcctggacggcggctacaccttcaccgag |
| | taccagaagaaccccgtgctggccgccaactccacccagttccgcgacccgaaggtcttctggtacgagccctcccagaagt |
| | ggatcatgaccgcggccaagtcccaggactacaagatcgagatctactcctccgacgacctgaagtcctggaagctggagtc |
| | cgcgttcgccaacgagggcttcctcggctaccagtacgagtgccccggcctgatcgaggtccccaccgagcaggaccccagc |
| | aagtcctactgggtgatgttcatctccatcaaccccggcgccccggccggcggctccttcaaccagtacttcgtcggcagct |
| | tcaacggcacccacttcgaggccttcgacaaccagtcccgcgtggtggacttcggcaaggactactacgccctgcagacctt |
| | cttcaacaccgacccgacctacgggagcgccctgggcatcgcgtgggcctccaactgggagtactccgccttcgtgcccacc |
| | aacccctggcgctcctccatgtccctcgtgcgcaagttctccctcaacaccgagtaccaggccaacccggagacggagctga |
| | tcaacctgaaggccgagccgatcctgaacatcagcaacgccggcccctggagccggttcgccaccaacaccacgttgacgaa |
| | ggccaacagctacaacgtcgacctgtccaacagcaccggcaccctggagttcgagctggtgtacgccgtcaacaccacccag |
| | acgatctccaagtccgtgttcgcggacctctccctctggttcaagggcctggaggaccccgaggagtacctccgcatgggct |
| | tcgaggtgtccgcgtcctccttcttcctggaccgcgggaacagcaaggtgaagttcgtgaaggagaacccctacttcaccaa |
| | ccgcatgagcgtgaacaaccagcccttcaagagcgagaacgacctgtcctactacaaggtgtacggcttgctggaccagaac |
| | atcctggagctgtacttcaacgacggcgacgtcgtgtccaccaacacctacttcatgaccaccgggaacgccctgggctccg |
| | tgaacatgacgacgggggtggacaacctgttctacatcgacaagttccaggtgcgcgaggtcaagtgactTAAGACGCCCGC |
| | GCGGCGCACCTGACCTGTTCTCTCGAGGGCGCCTGTTCTGCCTTGCGAAACAAGCCCCTGGAGCATGCGTGCATGATCGTCT |
| | CTGGCGCCCCGCCGCGCGGTTTGTCGCCCTCGCGGGCGCCGCGGCCGCGGGGGCGCATTGAAATTGTTGCAAACCCCACCTG |
| | ACAGATTGAGGGCCCAGGCAGGAAGGCGTTGAGATGGAGGTACAGGAGTCAAGTAACTGAAAGTTTTTATGATAACTAACAA |
| | CAAAGGGTCGTTTCTGGCCAGCGAATGACAAGAACAAGATTCCACATTTCCGTGTAGAGGCTTGCCATCGAATGTGAGCGGG |
| | CGGGCCGCGGACCCGACAAAACCCTTACGACGTGGTAAGAAAAACGTGGCGGGCACTGTCCCTGTAGCCTGAAGACCAGCAG |
| | GAGACGATCGGAAGCATCACAGCACAACCGGTgcagcagcagctcggatagtatcgacacactctggacgctggtcgtgtga |
| | tggactgttgccgccacacttgctgccttgacctgtgaatatccctgccgcttttatcaaacagcctcagtgtgtttgatct |
| | tgtgtgtacgcgcttttgcgagttgctagctgcttgtgctatttgcgaataccacccccagcatccccttccctcgtttcat |
| | atcgcttgcatcccaaccgcaacttatctacgctgtcctgctatccctcagcgctgctcctgctcctgctcactgcccctcg |
| | cacagccttggtttgggctccgcctgtattctcctggtactgcaacc |
| | 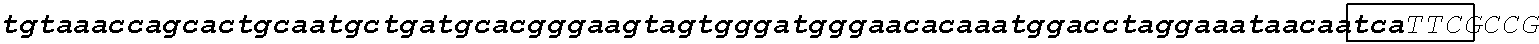 |
| | 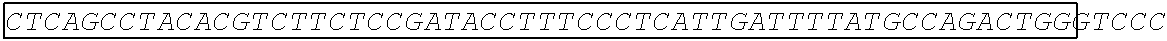 |
| | 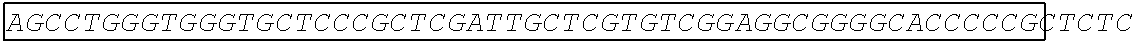 |
| | 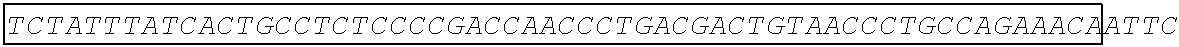 |
| | 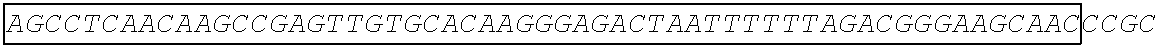 |
| | 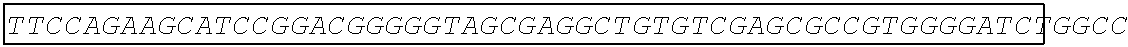 |
| | 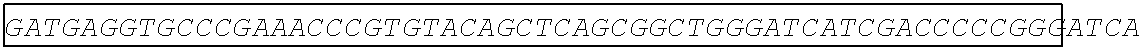 |
| | 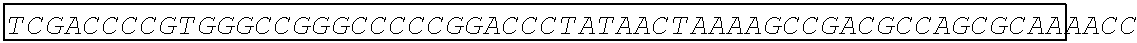 |
| | 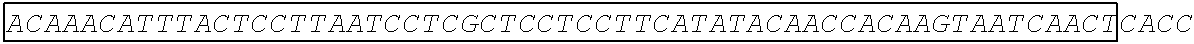 |
| | 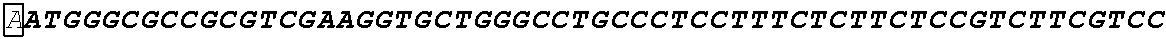 |
| | TGTACTGGTCTCTGCCCATCTTCGCGGTGCTCTACCGCATCCGATTCGCCAAGATGATCGGCAAGCGCAACGACGTGCTGGA |
| | CTGGGCCAAGTCTCTCGTGCGGTTTTTCGGCATCACCGTCCTGCGCCAGGGCCAAGAGCAACTGTACACGGGTGGCCCCTGC |
| | CTGTACCTGTGCAACCACCGCTCGTGGGCCGATTTCTTTCTGGACGTGTACCTGACCGAGGGCCACGCGGCCGTCATGTCGC |
| | GCTGGCTGGTCTACTTCGTCTTCCCGGTCTTCTGCACGTCCTGCCTGATCCTCAAGACGATTGTCCTCTTCAAGCGCGGCAC |
| | CATCCGCGACAAGGAGGGCTTTAACCGCTGGCTGGACGCCGTCCTGGCCCGCTCGAACGTGCCTGGCCTCCTGGTGTTCCCG |
| | GAGGGCCACCGCAGCCTGCGTCCCGCCTCGCTGCCACTGAAGCGGGGCATGCTGCATTACGCCTACGGCCGGAAGCTGGCGA |
| | TCCAGATGGTGGTCACGCGTGGCAAGGAGGAGGTGATGTCCGAGAAGACCATGGCCGTCCGCTTCGGCCGCACGCTGGTCAC |
| | GACCTACTCCAAGGTCCTGCGCCCGGCCGACTACCCTGACTTTGAGGCGTTCTTTGCGGACCTCCAGTCCACCTGGGACGGC |
| | TGCTGGGAGGCCGCGTACCGGAAGCCTCGGGAGGACGCCGTGCGGCACTCTCTGGGCGCGGCCCCCGGCTTTCACTACCCCT |
| | TTGCGACGCGTGTGGCGCAACTGGGCGCCACCCTGGGGTCCATCGCCCTGTTCGGCCTGGTCCTGGTGGGCTGCTGGCGTGG |
| | CCTGGCCGCTGCCCTGGCGGCCACGGGCGCCGCGCAGCAAGCGGTCGCCGCGCTCCTGGCCGTCTGGGTGGGTGGCTCCATG |
| | CTCCGTTCGTTCCTGTGAgtaccacgcccgcgcggcgcacctgacctgttctctcgagggcgcctgttctgccttgcgaaac |
| | aagcccctggagcatgcgtgcatgatcgtctctggcgccccgccgcgcggtttgtcgccctcgcgggcgccccggccgcggg |
| | ggcgcattgaaattgttgcaaaccccacctgacagattgagggcccaggcaggaaggcgttgagatggaggtacaggagtca |
| | agtaactgaaagtttttatgataactaacaacaaagggtcgtttctggccagcgaatgacaaga acaagattccacatttc |
| | cgtgtagaggcttgccatcgaatgtgagcgggcgggccgcggacccgacaaaacccttacgacgtggtaagaaaaacgtggc |
| | gggcactgtccctgtagcctgaagaccagcaggagacgatcggaagcatcacagcacaggccggcccgcgtctcgaacagag |
| | cgcggcggccgcatcgatacctgcaggacagcgccatgccacgccctttgatggcttcaagtacgattacggtgttggattg |
| | tgtgtttgttgcgtagtgtgcatggtttagaataatacacttgatttcttgctcacggcaatctcggcttgtccgcaggttc |
| | aaccccatttcggagtctcaggtcagccgcgcaatgaccagccgctacttcaaggacttgcacgacaacgccgaggtgagct |
| | atgtttaggacttgattggaaattgtcgtcgacgcatattcgcgctccgcgacagcacccaagcaaaatgtcaagtgcgttc |
| | cgatttgcgtccgcaggtcgatgttgtgatcgtcggcgccggatccgccggtctgtcctgcgcttacgagctgaccaagcac |
| | cctgacgtccgggtacgcgagctgagattcgattagacataaattgaagattaaacccgtagaaaaatttgatggtcgcgaa |
| | actgtgctcgattgcaagaaattgatcgtcctccactccgcaggtcgccatcatcgagcagggcgttgctcccggcggcggc |
| | gcctggctggggggacagctgttctcggccatgtgtgtacgtagaaggatgaatttcagctggttttcgttgcacagctgtt |
| | tgtgcatgatttgtttcagactattgttgaatgtttttagatttcttaggatgcatgatttgtctgcatgcgact |

The construct can be written as 5′Thia4::CrTUB2:ScSUC2:PmPGH:CvNR:PmG3PDH:VaulLPAAT2:PmPGH::3′Thia4. Proceeding in the 5′ to 3′ direction, bold, lowercase sequences represent genomic DNA from CHK22 that permit targeted integration at the Thia4 locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR that enables amplification of the VaulLPAAT2 gene, is indicated by uppercase underlined text followed by a linker, indicated by lowercase, bold italics, which contains a C. vulgaris nitrate reductase 3′-UTR. The P. moriformis PmG3PDH promoter, indicated by boxed italicized text, drive the expression of the VaulLPAAT2. The initiator ATG and terminator TGA codons of the VaulLPAAT2 are indicated by uppercase, bold italics, while the remainder of the gene is indicated by bold italics. The PmPGH 3′-UTR is indicated by lowercase underlined text followed by the CHK22 Thia4 genomic region indicated by bold, lowercase text.

Table 97 shows the fatty acid/TAG profiles of seven primary transformants of CHK22 transformed with pCHK1222. The P. moriformis base strain CHK22 is shown as a non-transgenic control. Strains were grown for 120 hrs in 50-mL conical tube at 200 rpm at 28° C. in lipid production medium. Biomass was harvested, lyophilized to dryness, and subjected to direct transesterification to generate FAMEs for subsequent quantitation and characterization b GC/FID. TAG analysis was conducted using the LCMS method as described above.

TABLE 97
Screen of primary transformants of CHK22 with pCHK1222.

| | FAME Profiles | | | | | | | | | TAG Profile |
|---|---|---|---|---|---|---|---|---|---|---|
| | | | | | | | C18:3 | | | m/z 577/603 |
| Sample | C12:0 | C14:0 | C16:0 | C18:0 | C18:1 | C18:2 | alpha | C20:0 | C20:1 | (OP:OO) |
| pCHK1222-1 | 0.04 | 1.43 | 28.52 | 2.75 | 57.09 | 7.99 | 0.52 | 0.19 | 0.00 | 3.5 |
| pCHK1222-2 | 0.04 | 1.40 | 28.30 | 2.70 | 56.85 | 8.52 | 0.52 | 0.19 | 0.00 | 3.6 |
| pCHK1222-3 | 0.04 | 1.35 | 27.70 | 2.96 | 59.21 | 6.63 | 0.48 | 0.23 | 0.00 | 1.5 |
| pCHK1222-4 | 0.03 | 1.13 | 28.97 | 1.82 | 57.29 | 8.40 | 0.47 | 0.14 | 0.03 | 4.6 |
| pCHK1222-5 | 0.04 | 1.45 | 28.48 | 3.17 | 57.53 | 7.29 | 0.50 | 0.22 | 0.00 | 3.1 |
| CHK22 Control | 0.04 | 1.4 | 25.8 | 2.69 | 61.83 | 6.51 | 0.36 | 0.16 | 0 | 1.5 |

### Example 47: Expressing Chlamydomonas sp. CCAC2762_B (Ch_CCAC2762_LPAAT2) in P. moriformis Strain CHK22 Using the PmG3PDH Promoter

Ch_CCAC2762_LPAAT2 was introduced into a P. moriformis strain CHK22. The expression construct pCHK1224 contained 5′ and 3′ homology arms to permit its targeted integration into the genome and is shown in Table 98.

TABLE 98
Integrative sequences for the transformation of P. moriformis with pCHK1224
encoding Ch_CCAC2762_LPAAT2.

| SEQ ID | |
|---|---|
| NO: | Nucleotide Sequence |
| 77 | ccctcaactgcgacgctgggaaccttctccgggcaggcgatgtgcgtgggtttgcctccttggcacggctctacaccgtcga |
| | gtacgccatgaggcggtgatggctgtgtcggttgccacttcgtccagagacggcaagtcgtccatcctctgcgtgtgtggcg |
| | cgacgctgcagcagtccctctgcagcagatgagcgtgactttggccatttcacgcactcgagtgtacacaatccatttttct |
| | taaagcaaatgactgctgattgaccagatactgtaacgctgatttcgctccagatcgcacagatagcgaccatgttgctgcg |
| | tctgaaaatctggattccgaattcgaccctggcgctccatccatgcaacagatggcgacacttgttacaattcctgtcaccc |
| | atcggcatggagcaggtccacttagattcccgatcacccacgcacatctcgctaatagtcattcgttcgtgtcttcgatcaa |
| | tctcaagtgagtgtgcatggatcttggttgacgatgcggtatgggtttgcgccgctggctgcagggtctgcccaaggcaagc |
| | taacccagctcctctccccgacaatactctcgcaggcaaagccggtcacttgccttccagattgccaataaactcaattatg |
| | gcctctgtcatgccatccatgggtctgatgaatggtcacgctcgtgtcctgaccgtt |
| | 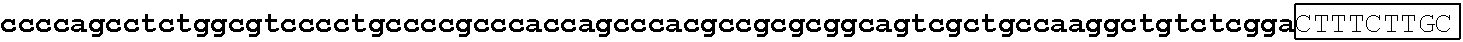 |
| | 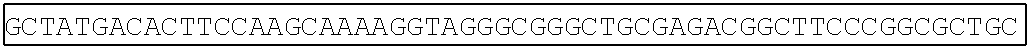 |
| | 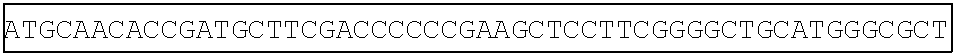 |
| | 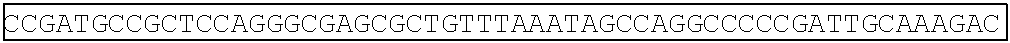 |
| | 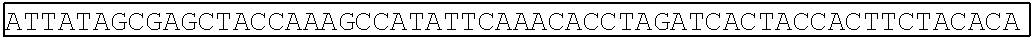 |
| | 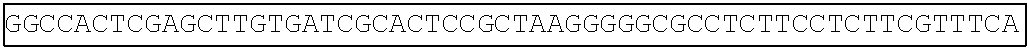 |
| | 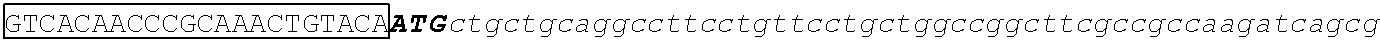 |
| | cctccatgacgaacgagacgtccgaccgccccctggtgcacttcacccccaacaagggctggatgaacgaccccaacggcct |
| | gtggtacgacgagaaggacgccaagtggcacctgtacttccagtacaacccgaacgacaccgtctgggggacgcccttgttc |
| | tggggccacgccacgtccgacgacctgaccaactgggaggaccagcccatcgccatcgccccgaagcgcaacgactccggcg |
| | ccttctccggctccatggtggtggactacaacaacacctccggcttcttcaacgacaccatcgacccgcgccagcgctgcgt |
| | ggccatctggacctacaacaccccggagtccgaggagcagtacatctcctacagcctggacggcggctacaccttcaccgag |
| | taccagaagaaccccgtgctggccgccaactccacccagttccgcgacccgaaggtcttctggtacgagccctcccagaagt |
| | ggatcatgaccgcggccaagtcccaggactacaagatcgagatctactcctccgacgacctgaagtcctggaagctggagtc |
| | cgcgttcgccaacgagggcttcctcggctaccagtacgagtgccccggcctgatcgaggtccccaccgagcaggaccccagc |
| | aagtcctactgggtgatgttcatctccatcaaccccggcgccccggccggcggctccttcaaccagtacttcgtcggcagct |
| | tcaacggcacccacttcgaggccttcgacaaccagtcccgcgtggtggacttcggcaaggactactacgccctgcagacctt |
| | cttcaacaccgacccgacctacgggagcgccctgggcatcgcgtgggcctccaactgggagtactccgccttcgtgcccacc |
| | aacccctggcgctcctccatgtccctcgtgcgcaagttctccctcaacaccgagtaccaggccaacccggagacggagctga |
| | tcaacctgaaggccgagccgatcctgaacatcagcaacgccggcccctggagccggttcgccaccaacaccacgttgacgaa |
| | ggccaacagctacaacgtcgacctgtccaacagcaccggcaccctggagttcgagctggtgtacgccgtcaacaccacccag |
| | acgatctccaagtccgtgttcgcggacctctccctctggttcaagggcctggaggaccccgaggagtacctccgcatgggct |
| | tcgaggtgtccgcgtcctccttcttcctggaccgcgggaacagcaaggtgaagttcgtgaaggagaacccctacttcaccaa |
| | ccgcatgagcgtgaacaaccagcccttcaagagcgagaacgacctgtcctactacaaggtgtacggcttgctggaccagaac |
| | atcctggagctgtacttcaacgacggcgacgtcgtgtccaccaacacctacttcatgaccaccgggaacgccctgggctccg |
| | tgaacatgacgacgggggtggacaacctgttctacatcgacaagttccaggtgcgcgaggtcaagtgactTAAGACGCCCG |
| | CGCGGCGCACCTGACCTGTTCTCTCGAGGGCGCCTGTTCTGCCTTGCGAAACAAGCCCCTGGAGCATGCGTGCATGATCG |
| | TCTCTGGCGCCCCGCCGCGCGGTTTGTCGCCCTCGCGGGCGCCGCGGCCGCGGGGGC |
| | GCATTGAAATTGTTGCAAACCCCACCTGACAGATTGAGGGCCCAGGCAGGAAGGCGT |
| | TGAGATGGAGGTACAGGAGTCAAGTAACTGAAAGTTTTTATGATAACTAACAACAAA |
| | GGGTCGTTTCTGGCCAGCGAATGACAAGAACAAGATTCCACATTTCCGTGTAGAGGC |
| | TTGCCATCGAATGTGAGCGGGCGGGCCGCGGACCCGACAAAACCCTTACGACGTGGT |
| | AAGAAAAACGTGGCGGGCACTGTCCCTGTAGCCTGAAGACCAGCAGGAGACGATCG |
| | GAAGCATCACAGCACAACCGGTgcagcagcagctcggatagtatcgacacactctggacgctggtcgtgtgatggactgttg |
| | ccgccacacttgctgccttgacctgtgaatatccctgccgcttttatcaaacagcctcagtgtgtttgatcttgtgtgtacg |
| | cgcttttgcgagttgctagctgcttgtgctatttgcgaataccacccccagcatccccttccctcgtttcatatcgcttgca |
| | tcccaaccgcaacttatctacgc |
| | 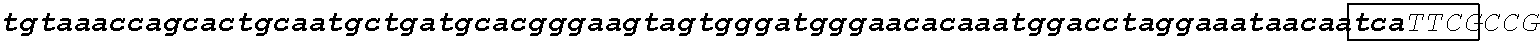 |
| | 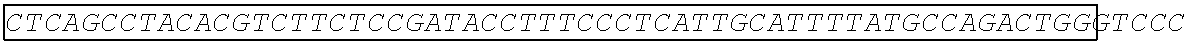 |
| | 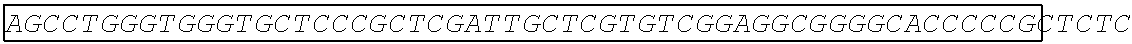 |
| | 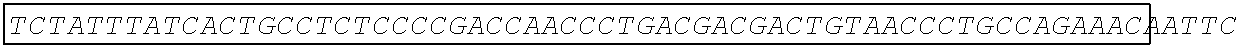 |
| | 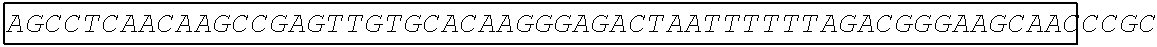 |
| | 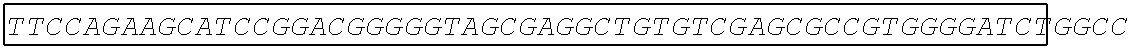 |
| | 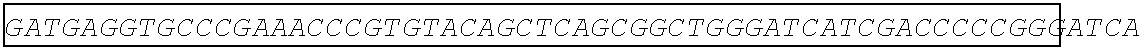 |
| | 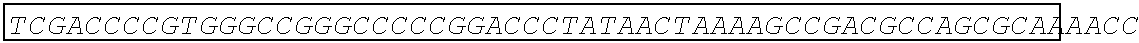 |
| | 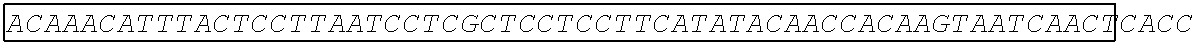 |
| | 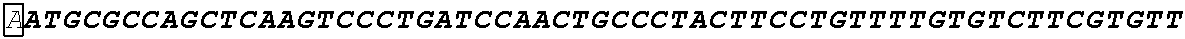 |
| | CTACTGGTCCTTCCCCATCGGCTGCCTCACCCATCACATCCACTTCCTGAAGTTTTCCGG |
| | CAAGCGCAACGACATGCACACCTGGTCTGAGACCATGTGCTCTTTCTTTGGCATCCGCT |
| | TCGTGAAGTACGGTGAGGAGAAGCTCAGCAAGGGTTCCCGCAACATCCTCTACCTGTCC |
| | AACCACCGCTCGTGGGCCGACTTTTTCGTCGATGTCTACCTGACCGAGGGCAACGCCGC |
| | GATGCTCAGCCGCATGCTGGTCTTCTTTGTCTTTCCCATCTTCATGACCTCGGTCGTGGT |
| | CCTGAAGGGCATTATCCTGTTCAAGCGCGGCACGATCGCCGACAAGGAGAAGTTCAAC |
| | GCCTGGCTGGATGTGATGCTGGAACGATCGCCCATCCCCGGAATGCTGGTCTACCCCG |
| | AGGGCCATCGGTCGACGCGGCCCCACAGCCTCCCGCTGAAGCGCGGCATGCTGTACTA |
| | CGCCCACAGCCGGGGCATGCCCGTGCAGATCATTATCTCGAACGGCAAGGAGGGCGTC |
| | ATGTCGGAGAAGCAGCGCCGAGCGGGCTTTGGCAAGACGGTCGTGGTCGGCTTCTCTT |
| | CGGTGATTGACACCGCGGCCTACAAGGGCAACGTCGAGGGCTTTATGGCGCGAGTGCA |
| | GGAGGAGTGGAACAAGCTGTGGGGTCAGGTCTACTCCGCCGACCCGGCGGGCCTGCCC |
| | CCTCTGCGCCAAGATCCCCCGATGCTGGACTACGCGGCCAGCATGCGAACGCAGCAAC |
| | TGGCCATCGCCGTCTTCTCCATGTTCGCCCTGGCTGGCGTGCTGTGGGTGACGTGGCG |
| | CATGTTCGCGTGGCTGACCGCCCTGGCCGGGCCCCTGCAACACGCCCTGGCCGTGCTC |
| | CTGCTCACCTGGTTCGCTGCGTCCATTATCGCGGGCGTGATGGGCCCGTCGCTCCCGAC |
| | CCACTCTTCCTCGCTCCAGAATCCCCCTTCTCTGAAGGCCGACAAGAGCGTGTCGGCCG |
| | CTGCCGCTGCCGCTGCCGCGGCCTCCGGCGACGGGAGCCTCCACGAGGCCCCTAGCGG |
| | CAAGGCCCCGGCTGCCGCTGCCACCCAGAACCACAACCACCAACACTGAgtaccacgcccgcg |
| | cggcgcacctgacctgttctctcgagggcgcctgttctgccttgcgaaacaagcccctggagcatgcgtgcatgatcgtctc |
| | tggcgccccgccgcgcggtttgtcgccctcgcgggcgccccggccgcgggggcgcattgaaattgttgcaaaccccacctga |
| | cagattgagggcccaggcaggaaggcgttgagatggaggtacaggagtcaagtaactgaaagtttttatgataactaacaac |
| | aaagggtcgtttctggccagcgaatgacaagaacaagattccacatttccgtgtagaggcttgccatcgaatgtgagcgggg |
| | ggccgcggacccgacaaaacccttacgacgtggtaagaaaaacgtggcgggcactgtccctgtagcctgaagaccagcagga |
| | gacgatcggaagcatcacagcacaggccggcccgcgtctcgaacagagcgcggcggccgcatcgatacctgcaggacagcgc |
| | catgccacgccctttgatggcttcaagtacgattacggtgttggattgtgtgtttgttgcgtagtgtgcatggtttagaata |
| | atacacttgatttcttgctcacggcaatctcggcttgtccgcaggttcaaccccatttcggagtctcaggtcagccgcgcaa |
| | tgaccagccgctacttcaaggacttgcacgacaacgccgaggtgagctatgtttaggacttgattggaaattgtcgtcgacg |
| | catattcgcgctccgcgacagcacccaagcaaaatgtcaagtgcgttccgatttgcgtccgcaggtcgatgttgtgatcgtc |
| | ggcgccggatccgccggtctgtcctgcgcttacgagctgaccaagcaccctgacgtccgggtacgcgagctgagattcgatt |
| | agacataaattgaagattaaacccgtagaaaaatttgatggtcgcgaaactgtgctcgattgcaagaaattgatcgtcctcc |
| | actccgcaggtcgccatcatcgagcagggcgttgctcccggcggcggcgcctggctggggggacagctgttctcggccatgt |
| | gtgtacgtagaaggatgaatttcagctggttttcgttgcacagctgtttgtgcatgatttgtttcagactattgttgaatgt |
| | ttttagatttcttaggatgcatgatttgtctgcatgcgact |

The construct can be written as 5′Thia4::CrTUB2:ScSUC2:PmPGH:CvNR:PmG3PDH:Ch_CCAC2762_LPAAT2:PmPGH::3′Thia4. Proceeding in the 5′ to 3′ direction, bold, lowercase sequences represent genomic DNA from CHK22 that permit targeted integration at the Thia4 locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR that enables amplification of the Ch_CCAC2762_LPAAT2 gene, is indicated by uppercase underlined text followed by a linker, indicated by lowercase, bold italics, which contains a C. vulgaris nitrate reductase 3′-UTR. A PmG3PDH promoter from P. moriformis, indicated by boxed italicized text, drive the expression of the Ch_CCAC2762_LPAAT2. The initiator ATG and terminator TGA codons of the Ch_CCAC2762_LPAAT2 are indicated by uppercase, bold italics, while the remainder of the gene is indicated by bold italics. The PmPGH 3′-UTR is indicated by lowercase underlined text followed by the CHK22 Thia4 genomic region indicated by bold, lowercase text.

Table 99 shows the fatty acid/TAG profiles of seven primary transformants of CHK22 transformed with pCHK1224. The P. moriformis base strain CHK22 is shown as a non-transgenic control. Strains were grown for 120 hrs in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 99
Screen of primary transformants of CHK22 with pCHK1224.

| | FAME Profiles | | | | | | | | | TAG Profile |
|---|---|---|---|---|---|---|---|---|---|---|
| | | | | | | | C18:3 | | | m/z 577/603 |
| Sample | C12:0 | C14:0 | C16:0 | C18:0 | C18:1 | C18:2 | alpha | C20:0 | C20:1 | (OP:OO) |
| pCHK1224-1 | 0.03 | 1.25 | 29.39 | 2.29 | 57.45 | 7.55 | 0.48 | 0.21 | 0.00 | 4.59 |
| pCHK1224-2 | 0.03 | 1.06 | 25.88 | 3.15 | 61.04 | 6.93 | 0.47 | 0.27 | 0.00 | 1.65 |
| pCHK1224-3 | 0.03 | 1.18 | 26.12 | 3.21 | 60.96 | 6.52 | 0.51 | 0.29 | 0.00 | 1.47 |
| pCHK1224-4 | 0.05 | 2.02 | 27.54 | 3.09 | 57.62 | 7.54 | 0.63 | 0.23 | 0.00 | 3.83 |
| pCHK1224-5 | 0.03 | 1.36 | 27.00 | 3.18 | 59.02 | 7.34 | 0.59 | 0.28 | 0.00 | 1.83 |
| pCHK1224-6 | 0.03 | 1.23 | 26.13 | 3.37 | 60.46 | 6.86 | 0.52 | 0.30 | 0.08 | 1.91 |
| pCHK1224-7 | 0.03 | 1.23 | 28.61 | 2.97 | 55.28 | 9.69 | 1.33 | 0.39 | 0.00 | 2.23 |
| pCHK1224-8 | 0.03 | 1.22 | 28.38 | 2.94 | 58.67 | 6.84 | 0.46 | 0.24 | 0.05 | 3.12 |
| CHK22 Control | 0.04 | 1.4 | 25.8 | 2.69 | 61.83 | 6.51 | 0.36 | 0.16 | 0 | 1.5 |

### Example 48: Expressing Dunaliella salina (DsalLPAAT2) in P. moriformis Strain CHK22 Using the PmG3PDG Promoter

DsalLPAAT2 was introduced into a P. moriformis strain CHK22. The expression construct pCHK1225 contained 5′ and 3′ homology arms to permit its targeted integration into the genome and is shown in Table 100.

TABLE 100
Integrative sequences for the transformation of P. moriformis with pCHK1225
encoding DsalLPAAT2.

| SEQ ID | |
|---|---|
| NO: | Nucleotide Sequence |
| 78 | ccctcaactgcgacgctgggaaccttctccgggcaggcgatgtgcgtgggtttgcctccttggcacggctctacaccgtcga |
| | gtacgccatgaggcggtgatggctgtgtcggttgccacttcgtccagagacggcaagtcgtccatcctctgcgtgtgtggcg |
| | cgacgctgcagcagtccctctgcagcagatgagcgtgactttggccatttcacgcactcgagtgtacacaatccatttttct |
| | taaagcaaatgactgctgattgaccagatactgtaacgctgatttcgctccagatcgcacagatagcgaccatgttgctgcg |
| | tctgaaaatctggattccgaattcgaccctggcgctccatccatgcaacagatggcgacacttgttacaattcctgtcaccc |
| | atcggcatggagcaggtccacttagattcccgatcacccacgcacatctcgctaatagtcattcgttcgtgtcttcgatcaa |
| | tctcaagtgagtgtgcatggatcttggttgacgatgcggtatgggtttgcgccgctggctgcagggtctgcccaaggcaagc |
| | taacccagctcctctccccgacaatactctcgcaggcaaagccggtcacttgccttccagattgccaataaactcaattatg |
| | gcctctgtcatgccatccatgggtctgatgaatggtcacgctcgtgtcctgaccgtt |
| | 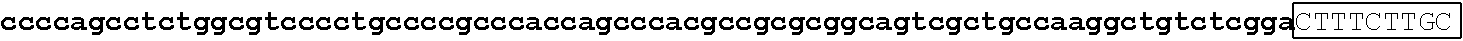 |
| | 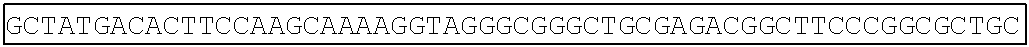 |
| | 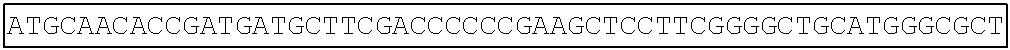 |
| | 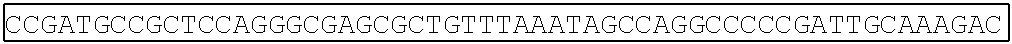 |
| | 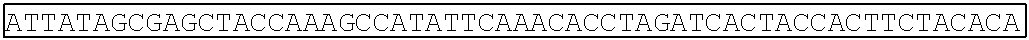 |
| | 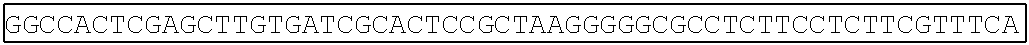 |
| | 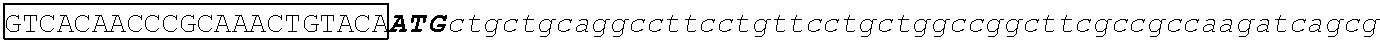 |
| | cctccatgacgaacgagacgtccgaccgccccctggtgcacttcacccccaacaagggctggatgaacgaccccaacggcct |
| | gtggtacgacgagaaggacgccaagtggcacctgtacttccagtacaacccgaacgacaccgtctgggggacgcccttgttc |
| | tggggccacgccacgtccgacgacctgaccaactgggaggaccagcccatcgccatcgccccgaagcgcaacgactccggcg |
| | ccttctccggctccatggtggtggactacaacaacacctccggcttcttcaacgacaccatcgacccgcgccagcgctgcgt |
| | ggccatctggacctacaacaccccggagtccgaggagcagtacatctcctacagcctggacggcggctacaccttcaccgag |
| | taccagaagaaccccgtgctggccgccaactccacccagttccgcgacccgaaggtcttctggtacgagccctcccagaagt |
| | ggatcatgaccgcggccaagtcccaggactacaagatcgagatctactcctccgacgacctgaagtcctggaagctggagtc |
| | cgcgttcgccaacgagggcttcctcggctaccagtacgagtgccccggcctgatcgaggtccccaccgagcaggaccccagc |
| | aagtcctactgggtgatgttcatctccatcaaccccggcgccccggccggcggctccttcaaccagtacttcgtcggcagct |
| | tcaacggcacccacttcgaggccttcgacaaccagtcccgcgtggtggacttcggcaaggactactacgccctgcagacctt |
| | cttcaacaccgacccgacctacgggagcgccctgggcatcgcgtgggcctccaactgggagtactccgccttcgtgcccacc |
| | aacccctggcgctcctccatgtccctcgtgcgcaagttctccctcaacaccgagtaccaggccaacccggagacggagctga |
| | tcaacctgaaggccgagccgatcctgaacatcagcaacgccggcccctggagccggttcgccaccaacaccacgttgacgaa |
| | ggccaacagctacaacgtcgacctgtccaacagcaccggcaccctggagttcgagctggtgtacgccgtcaacaccacccag |
| | acgatctccaagtccgtgttcgcggacctctccctctggttcaagggcctggaggaccccgaggagtacctccgcatgggct |
| | tcgaggtgtccgcgtcctccttcttcctggaccgcgggaacagcaaggtgaagttcgtgaaggagaacccctacttcaccaa |
| | ccgcatgagcgtgaacaaccagcccttcaagagcgagaacgacctgtcctactacaaggtgtacggcttgctggaccagaac |
| | atcctggagctgtacttcaacgacggcgacgtcgtgtccaccaacacctacttcatgaccaccgggaacgccctgggctccg |
| | tgaacatgacgacgggggtggacaacctgttctacatcgacaagttccaggtgcgcgaggtcaagtgactTAAGACGCCCGC |
| | GCGGCGCACCTGACCTGTTCTCTCGAGGGCGCCTGTTCTGCCTTGCGAAACAAGCCCCTGGAGCATGCGTGCATGATCG |
| | TCTCTGGCGCCCCGCCGCGCGGTTTGTCGCCCTCGCGGGCGCCGCGGCCGCGGGGGC |
| | GCATTGAAATTGTTGCAAACCCCACCTGACAGATTGAGGGCCCAGGCAGGAAGGCGT |
| | TGAGATGGAGGTACAGGAGTCAAGTAACTGAAAGTTTTTATGATAACTAACAACAAA |
| | GGGTCGTTTCTGGCCAGCGAATGACAAGAACAAGATTCCACATTTCCGTGTAGAGGC |
| | TTGCCATCGAATGTGAGCGGGCGGGCCGCGGACCCGACAAAACCCTTACGACGTGGT |
| | AAGAAAAACGTGGCGGGCACTGTCCCTGTAGCCTGAAGACCAGCAGGAGACGATCG |
| | GAAGCATCACAGCACAACCGGTgcagcagcagctcggatagtatcgacacactctggacgctggtcgtgtgatggactgttg |
| | ccgccacacttgctgccttgacctgtgaatatccctgccgcttttatcaaacagcctcagtgtgtttgatcttgtgtgtacg |
| | cgcttttgcgagttgctagctgcttgtgctatttgcgaataccacccccagcatccccttccctcgtttcatatcgcttgca |
| | tcccaaccgcaacttatctacgctgtcctgctatccctcagcgctgctcctgctcctgctcactgcccctcgcacagccttg |
| | gtttgggctccgcctgtattctcctggtactgcaacc |
| | 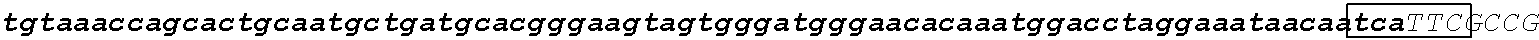 |
| | 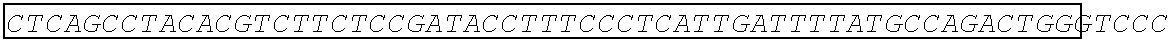 |
| | 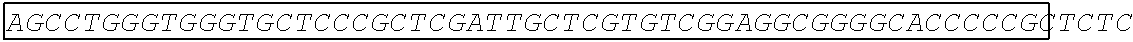 |
| | 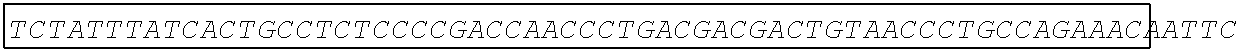 |
| | 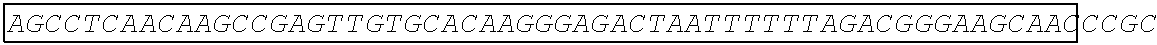 |
| | 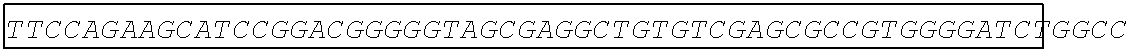 |
| | 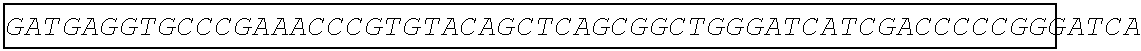 |
| | 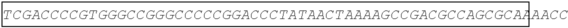 |
| | 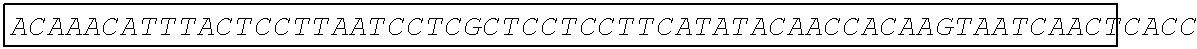 |
| | 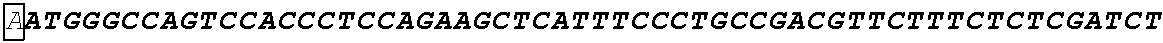 |
| | TCCTGATGTACTGGAGCCTGCCCCTCTGCACCCTGTGTGGCTACCTGCGCCTCCTGGAC |
| | TTTAAGTCCACGCGCAAGGACATGTACAACTGGTGCCGCTTCATGACGCGTCTCTTCCG |
| | TATCAAGGTCAAGAAGTACGGCTCCAAGTCGCTCCGCAAGGACGCGGGTCGGCATATC |
| | CTGTACCTGTGCAACCATCGTAGCTGGACGGACTTTTTCCTCGACGCTTACCTGACCGA |
| | GGGCAACGCCTCCCTGATGTCCCGTTGGCTCGTGTTTGCGGTCTTTCCGGTCTTCATCG |
| | TGTCGGTGATCGTCCTCCGGGGAGTCATCCTCTTCAAGCGCGGCACCATTCTGGACAAG |
| | GAGAAGTTTAATCGGTGGCTGGACTCGAAGATCCTGTCGACCCCGATCCCCGGCGTCCT |
| | CGTCTACCCCGAGGGCCACCGCTCTCTGGCCGACAAGTCGCTGCCACTCAAGCGCGGC |
| | ATGCTCTACTACGCCCACTCCCGAAAGTTCCCAGTGCAAATCATTATCTCCCGCAACAA |
| | GGAGGACGTCCTGAGCGAGAAGACCATGAAGGCCCACTTCGGCGCCACGATTATCACG |
| | GGCTGCTCCGACCTGATCGAGTCTGACGGCAAGGACATCAAGGTCTTTCTGTCCGAGAT |
| | CCAGGCCGCGTGGGATGACCTGTGGGAGAAGGTGTACGCGGCCGACGCGACCGCTCTG |
| | CCACCCCTGGAGCAGTCGAACATCCCCGAGTTCGACTACAGCATGGGGATGCGTGTCA |
| | AGCAGCTCCTGGTCGTGTGCAGCTCGGTGCTGGTGTTTCTGGCGGCCATGGTCGTGTTT |
| | GGTAAGCTCTCTTGCCTGATCTACGCCGCGATGGGTGACACCGAGCGCAAGGTCCTGG |
| | TGACCCTCATCCTGTACGTCTGGTTTTTCGTGAGCATGCTCAACAGCCGGGCCCCGAGC |
| | GCCACGCAGTAGgtaccacgcccgcgcggcacctgacctgttctctcgagggcgcctgttctgccttgogaaacaagcccct |
| | ggagcatgcgtgcatgatcgtctctggcgccccgccgcgcggtttgtcgccctcgcgggcgccccggccgcgggggcgcatt |
| | gaaattgttgcaaaccccacctgacagattgagggcccaggcaggaaggcgttgagatggaggtacaggagtcaagtaactg |
| | aaagtttttatgataactaacaacaaagggtcgtttctggccagcgaatgacaagaacaagattccacatttccgtgtagag |
| | gcttgccatcgaatgtgagcggggggccgcggacccgacaaaacccttacgacgtggtaagaaaaacgtggcgggcactgtc |
| | cctgtagcctgaagaccagcaggagacgatcggaagcatcacagcacaggccggcccgcgtctcgaacagagcgcggcggcc |
| | gcatcgatacctgcaggacagcgccatgccacgccctttgatggcttcaagtacgattacggtgttggattgtgtgtttgtt |
| | gcgtagtgtgcatggtttagaataatacacttgatttcttgctcacggcaatctcggcttgtccgcaggttcaaccccattt |
| | cggagtctcaggtcagccgcgcaatgaccagccgctacttcaaggacttgcacgacaacgccgaggtgagctatgtttagga |
| | cttgattggaaattgtcgtcgacgcatattcgcgctccgcgacagcacccaagcaaaatgtcaagtgcgttccgatttgcgt |
| | ccgcaggtcgatgttgtgatcgtcggcgccggatccgccggtctgtcctgcgcttacgagctgaccaagcaccctgacgtcc |
| | gggtacgcgagctgagattcgattagacataaattgaagattaaacccgtagaaaaatttgatggtcgcgaaactgtgctcg |
| | attgcaagaaattgatcgtcctccactccgcaggtcgccatcatcgagcagggcgttgctcccggcggcggcgcctggctgg |
| | ggggacagctgttctcggccatgtgtgtacgtagaaggatgaatttcagctggttttcgttgcacagctgtttgtgcatgat |
| | ttgtttcagactattgttgaatgtttttagatttcttaggatgcatgatttgtctgcatgcgact |

The construct can be written as 5′Thia4::CrTUB2:ScSUC2:PmPGH:CvNR:PmG3PDH:Dsa/LPAAT2:PmPGH::3′Thia4. Proceeding in the 5′ to 3′ direction, bold, lowercase sequences represent genomic DNA from CHK22 that permit targeted integration at the Thia4 locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR that enables amplification of the DsalLPAAT2 gene, is indicated by uppercase underlined text followed by a linker, indicated by lowercase, bold italics, which contains a C. vulgaris nitrate reductase 3′-UTR. A PmG3PDG promoter from P. moriformis, indicated by boxed italicized text, drive the expression of the DsalLPAAT2. The initiator ATG and terminator TAG codons of the DsalLPAAT2 are indicated by uppercase, bold italics, while the remainder of the gene is indicated by bold italics. The PmPGH 3′-UTR is indicated by lowercase underlined text followed by the CHK22 Thia4 genomic reg ion indicated by bold, lowercase text.

Table 101 shows the fatty acid/TAG profiles of seven primary transformants of CHK22 transformed with pCHK1225. The P. moriformis base strain CHK22 is shown as a non-transgenic control. Strains were grown for 120 hrs in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as

TABLE 101
Screen of primary transformants of CHK22 with pCHK1225.

| | FAME Profiles | | | | | | | | | TAG Profile |
|---|---|---|---|---|---|---|---|---|---|---|
| | | | | | | | C18:3 | | | m/z 577/603 |
| Sample | C12:0 | C14:0 | C16:0 | C18:0 | C18:1 | C18:2 | alpha | C20:0 | C20:1 | (OP:OO) |
| pCHK1225-1 | 0.03 | 1.15 | 26.06 | 3.07 | 61.11 | 6.63 | 0.53 | 0.24 | 0.00 | 1.69 |
| pCHK1225-2 | 0.03 | 1.20 | 26.00 | 3.28 | 61.09 | 6.56 | 0.52 | 0.26 | 0.00 | 1.49 |
| pCHK1225-3 | 0.03 | 1.23 | 26.56 | 3.41 | 60.15 | 6.69 | 0.50 | 0.31 | 0.00 | 2.06 |
| pCHK1225-4 | 0.03 | 1.28 | 25.40 | 3.28 | 61.54 | 6.55 | 0.54 | 0.25 | 0.00 | 1.56 |
| pCHK1225-5 | 0.03 | 1.21 | 25.26 | 2.85 | 62.38 | 6.56 | 0.40 | 0.17 | 0.00 | 1.59 |
| pCHK1225-6 | 0.06 | 1.72 | 19.85 | 3.23 | 66.00 | 7.47 | 0.44 | 0.20 | 0.00 | 1.57 |
| pCHK1225-7 | 0.03 | 1.22 | 26.17 | 3.27 | 60.76 | 6.65 | 0.49 | 0.25 | 0.00 | 1.69 |
| CHK22 Control | 0.04 | 1.4 | 25.8 | 2.69 | 61.83 | 6.51 | 0.36 | 0.16 | 0 | 1.5 |

### Example 49: Expressing Microglena sp. YARC (MyarcLPAAT2) in P. moriformis Strain CHK22 Using the PmG3PDH Promoter

MyarcLPAAT2 was introduced into a P. moriformis strain CHK22. The expression construct pCHK1227 contained 5′ and 3′ homology arms to permit its targeted integration into the genome and is shown in Table 102.

TABLE 102
Integrative sequences for the transformation of P. moriformis with pCHK1227
encoding MyarcLPAAT2.

| SEQ ID | |
|---|---|
| NO: | Nucleotide Sequence |
| 79 | ccctcaactgcgacgctgggaaccttctccgggcaggcgatgtgcgtgggtttgcctccttggcacggctctacaccgtcga |
| | gtacgccatgaggcggtgatggctgtgtcggttgccacttcgtccagagacggcaagtcgtccatcctctgcgtgtgtggcg |
| | cgacgctgcagcagtccctctgcagcagatgagcgtgactttggccatttcacgcactcgagtgtacacaatccatttttct |
| | taaagcaaatgactgctgattgaccagatactgtaacgctgatttcgctccagatcgcacagatagcgaccatgttgctgcg |
| | tctgaaaatctggattccgaattcgaccctggcgctccatccatgcaacagatggcgacacttgttacaattcctgtcaccc |
| | atcggcatggagcaggtccacttagattcccgatcacccacgcacatctcgctaatagtcattcgttcgtgtcttcgatcaa |
| | tctcaagtgagtgtgcatggatcttggttgacgatgcggtatgggtttgcgccgctggctgcagggtctgcccaaggcaagc |
| | taacccagctcctctccccgacaatactctcgcaggcaaagccggtcacttgccttccagattgccaataaactcaattatg |
| | gcctctgtcatgccatccatgggtctgatgaatggtcacgctcgtgtcctgaccgtt |
| | 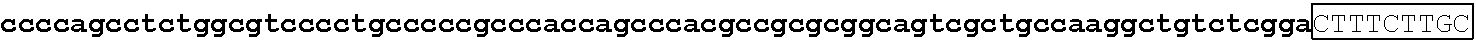 |
| | 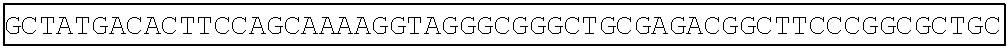 |
| | 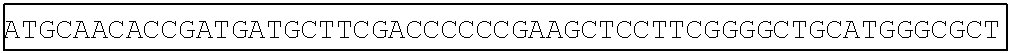 |
| | 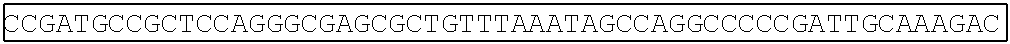 |
| | 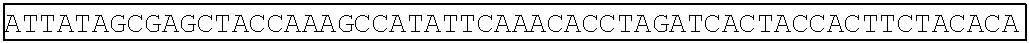 |
| | 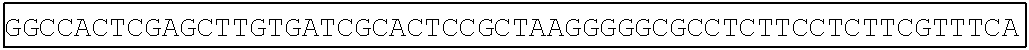 |
| | 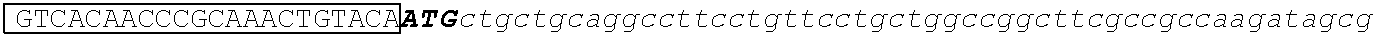 |
| | cctccatgacgaacgagacgtccgaccgccccctggtgcacttcacccccaacaagggctggatgaacgaccccaacggcct |
| | gtggtacgacgagaaggacgccaagtggcacctgtacttccagtacaacccgaacgacaccgtctgggggacgcccttgttc |
| | tggggccacgccacgtccgacgacctgaccaactgggaggaccagcccatcgccatcgccccgaagcgcaacgactccggcg |
| | ccttctccggctccatggtggtggactacaacaacacctccggcttcttcaacgacaccatcgacccgcgccagcgctgcgt |
| | ggccatctggacctacaacaccccggagtccgaggagcagtacatctcctacagcctggacggcggctacaccttcaccgag |
| | taccagaagaaccccgtgctggccgccaactccacccagttccgcgacccgaaggtcttctggtacgagccctcccagaagt |
| | ggatcatgaccgcggccaagtcccaggactacaagatcgagatctactcctccgacgacctgaagtcctggaagctggagtc |
| | cgcgttcgccaacgagggcttcctcggctaccagtacgagtgccccggcctgatcgaggtccccaccgagcaggaccccagc |
| | aagtcctactgggtgatgttcatctccatcaaccccggcgccccggccggcggctccttcaaccagtacttcgtcggcagct |
| | tcaacggcacccacttcgaggccttcgacaaccagtcccgcgtggtggacttcggcaaggactactacgccctgcagacctt |
| | cttcaacaccgacccgacctacgggagcgccctgggcatcgcgtgggcctccaactgggagtactccgccttcgtgcccacc |
| | aacccctggcgctcctccatgtccctcgtgcgcaagttctccctcaacaccgagtaccaggccaacccggagacggagctga |
| | tcaacctgaaggccgagccgatcctgaacatcagcaacgccggcccctggagccggttcgccaccaacaccacgttgacgaa |
| | ggccaacagctacaacgtcgacctgtccaacagcaccggcaccctggagttcgagctggtgtacgccgtcaacaccacccag |
| | acgatctccaagtccgtgttcgcggacctctccctctggttcaagggcctggaggaccccgaggagtacctccgcatgggct |
| | tcgaggtgtccgcgtcctccttcttcctggaccgcgggaacagcaaggtgaagttcgtgaaggagaacccctacttcaccaa |
| | ccgcatgagcgtgaacaaccagcccttcaagagcgagaacgacctgtcctactacaaggtgtacggcttgctggaccagaac |
| | atcctggagctgtacttcaacgacggcgacgtcgtgtccaccaacacctacttcatgaccaccgggaacgccctgggctccg |
| | tgaacatgacgacgggggtggacaacctgttctacatcgacaagttccaggtgcgcgaggtcaagtgactTAAGACGCCCGC |
| | GCGGCGCACCTGACCTGTTCTCTCGAGGGCGCCTGTTCTGCCTTGCGAAACAAGCCCCTGGAGCATGCGTGCATGATCG |
| | TCTCTGGCGCCCCGCCGCGCGGTTTGTCGCCCTCGCGGGCGCCGCGGCCGCGGGGGC |
| | GCATTGAAATTGTTGCAAACCCCACCTGACAGATTGAGGGCCCAGGCAGGAAGGCGT |
| | TGAGATGGAGGTACAGGAGTCAAGTAACTGAAAGTTTTTATGATAACTAACAACAAA |
| | GGGTCGTTTCTGGCCAGCGAATGACAAGAACAAGATTCCACATTTCCGTGTAGAGGC |
| | TTGCCATCGAATGTGAGCGGGCGGGCCGCGGACCCGACAAAACCCTTACGACGTGGT |
| | AAGAAAAACGTGGCGGGCACTGTCCCTGTAGCCTGAAGACCAGCAGGAGACGATCG |
| | GAAGCATCACAGCACAACCGGTgcagcagcagctcggatagtatcgacacactctggacgctggtcgtgtgatggactgttg |
| | ccgccacacttgctgccttgacctgtgaatatccctgccgcttttatcaaacagcctcagtgtgtttgatcttgtgtgtacg |
| | cgcttttgcgagttgctagctgcttgtgctatttgcgaataccacccccagcatccccttccctcgtttcatatcgcttgca |
| | tcccaaccgcaacttatctacgctgtcctgctatccctcagcgctgctcctgctcctgctcactgcccctcgcacagccttg |
| | gtttgggctccgcctgtattctcctggtactgcaacc |
| | 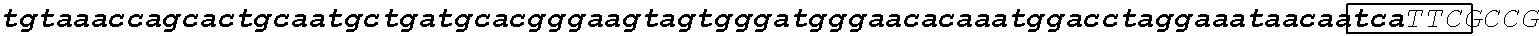 |
| | 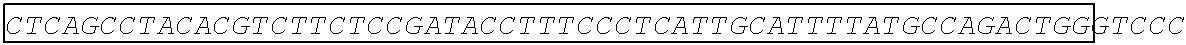 |
| | 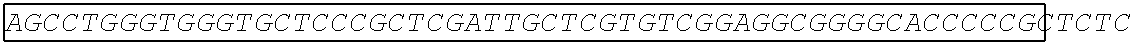 |
| | 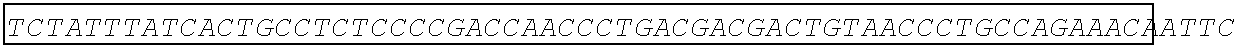 |
| | 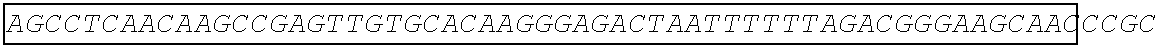 |
| | 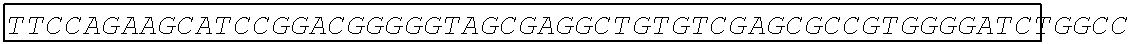 |
| | 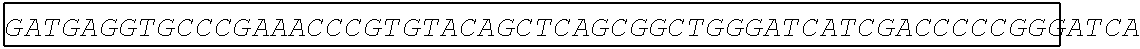 |
| | 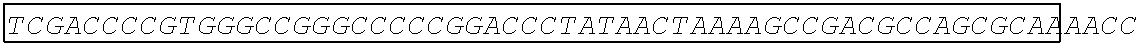 |
| | 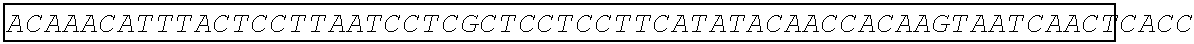 |
| | 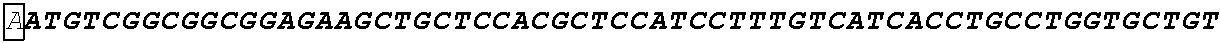 |
| | ACTGGTCCCTCCCTCTCTCCGTGCTGTTCGGCTACATCCGTGCCTTCCAGTGGACGGGT |
| | CCCCGAAACGACATGTACTCTTGGGGCCAGTTCATGTCCGACTTCTTTGGTGTGAAGTT |
| | CGTGCGCGTCGGCAACGAGAAGCTGTACAAGCCAAAGAAGGGCGCGAACGTCATCTAC |
| | CTCTGCAACCACCGCTCCTGGGCCGACTTTTTCTGCGACGTCTACCTCACCCAGGGCAA |
| | CGCCGCTCTCCTGTCGCGCATGCTGGTGTTTTTCATCTTTCCGTTTTTCATGTCCAGCGT |
| | CGTGATCCTGAAGGGCGTCGTGCTGTTCAAGCGGGGCAAGGTGGTCGACAAGGACGCG |
| | TTCAACAAGCGTCTCGACGGCAAGCTGGCGGACTCGCCCATCCCTGGCCTCCTGGTCTA |
| | CCCAGAGGGCACGCGGAGCCTGAAGGACCTGTCTCTGCCGCTGAAGCGCGGGATGCTC |
| | TACTTCGCGCATAGCCGCAACATTCCGGTGCAGATCGTGGTCAGCTCTGGAAAGGAGTA |
| | CGTCCTCACCGAGAAGAAGATCCGCGCCCGCATCGGCCGCACCGTCGTGTCCACCTTCT |
| | CCGCGAAGATCACCCCGTCCGACTTCCCATCGTGCGAGGCCTTCCTGGAGCACGTGCA |
| | AAAGGAGTGGGACCGCCTGTGGCCCGCCGTCTTCACGGCCGACGCTGACAAGGCCCCC |
| | GTCCTGGTGAACGAGGACATCCCGATGCACGACTACGCGAACACCATGCGATGGGCGC |
| | AGCTCGGAATGGCCGTGCTCTCTACCACGGTGGCGACGATCATGTTCCTCCTGAGCTGG |
| | AAGGTCGTGGACCTGATTTTCGGCATCTTCGGGCCCCTGAAGCAGGTCCTGTACATGCT |
| | CCTGTTCACCTGGGTGTCGGTGTCGCTCGTGGCGAGCTTCCTGGGGCCAAAGCATCTG |
| | CCCAAGGGGGTCCTGCCCTCCCCCGTCCTGCCCCTGCCCGTGGCGCGGCCACCGCCCC |
| | CGTCCCAGGAGCCGTCCAGCTCGGGGTCTAAGTGAgtaccacgcccgcgcggcgcacctgacctgttctctcggcgccccga |
| | gggcgcctgttctgccttgcgaaacaagcccctggagcatgcgtgcatgatcgtctctgggccccgccgcgcggtttgtcgc |
| | cctcgcgggccgcgggggcgcattgaaattgttgcaaaccccacctgacagattgagggcccaggcaggaaggcgttgagat |
| | ggaggtacaggagtcaagtaactgaaagtttttatgataactaacaacaaagggtcgtttctggccagcgaatgacaagaac |
| | aagattccacatttccgtgtagaggcttgccatcgaatgtgagcggggggccgcggacccgacaaaacccttacgacgtggt |
| | aagaaaaacgtgggggcactgtccctgtagcctgaagaccagcaggagacgatcggaagcatcacagcacaggccggcccgc |
| | gtctcgaacagagcgcggggccgcatcgatacctgcaggacagcgccatgccacgccctttgatggcttcaagtacgattac |
| | ggtgttggattgtgtgtttgttgcgtagtgtgcatggtttagaataatacacttgatttcttgctcacggcaatctcggctt |
| | gtccgcaggttcaaccccatttcggagtctcaggtcagccgcgcaatgaccagccgctacttcaaggacttgcacgacaacg |
| | ccgaggtgagctatgtttaggacttgattggaaattgtcgtcgacgcatattcgcgctccgcgacagcacccaagcaaaatg |
| | tcaagtgcgttccgatttgcgtccgcaggtcgatgttgtgatcgtcggcgccggatccgccggtctgtcctgcgcttacgag |
| | ctgaccaagcaccctgacgtccgggtacgcgagctgagattcgattagacataaattgaagattaaacccgtagaaaaattt |
| | gatggtcgcgaaactgtgctcgattgcaagaaattgatcgtcctccactccgcaggtcgccatcatcgagcagggcgttgct |
| | cccggcggcggcgcctggctggggggacagctgttctcggccatgtgtgtacgtagaaggatgaatttcagctggttttcgt |
| | tgcacagctgtttgtgcatgatttgtttcagactattgttgaatgtttttagatttcttaggatgcatgatttgtctgcatg |
| | cgact |

The construct can be written as 5′ Thia4::CrTUB2:ScSUC2:PmPGH:CvNR:PmG3PDHM:MyarcLPAAT2:PmPGH::3′Thia4. Proceeding in the 5′ to 3′ direction, bold, lowercase sequences represent genomic DNA from CHK22 that permit targeted integration at the Thia4 locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR that enables amplification of the MyarcLPAAT2 gene, is indicated by uppercase underlined text followed by a linker, indicated by lowercase, bold italics, which contains a C. vulgaris nitrate reductase 3′-UTR. A PmG3PDH promoter from P. moriformis, indicated by boxed italicized text, drive the expression of the MyarcLPAAT2. The initiator ATG and terminator TGA codons of the MyarcLPAAT2 are indicated by uppercase, bold italics, while the remainder of the gene is indicated by bold italics. The PmPGH 3′-UTR is indicated by lowercase underlined text followed by the CHK22 Thia4 genomic region indicated by bold, lowercase text.

Table 103 shows the fatty acid/TAG profiles of seven primary transformants of CHK22 transformed with pCHK1227. The P. moriformis base strain CHK22 is shown as a non-transgenic control. Strains were grown for 120 hrs in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 103
Screen of primary transformants of CHK22 with pCHK1227.

| | FAME Profiles | | | | | | | | | TAG Profile |
|---|---|---|---|---|---|---|---|---|---|---|
| | | | | | | | C18:3 | | | m/z 577/603 |
| Sample | C12:0 | C14:0 | C16:0 | C18:0 | C18:1 | C18:2 | alpha | C20:0 | C20:1 | (OP:OO) |
| pCHK1227-1 | 0.03 | 1.23 | 27.32 | 2.80 | 59.58 | 6.95 | 0.49 | 0.25 | 0.00 | 2.42 |
| pCHK1227-2 | 0.03 | 1.24 | 27.17 | 2.82 | 59.77 | 6.87 | 0.49 | 0.25 | 0.00 | 2.48 |
| pCHK1227-3 | 0.03 | 1.24 | 27.37 | 2.74 | 59.53 | 6.98 | 0.49 | 0.24 | 0.00 | 2.49 |
| pCHK1227-4 | 0.03 | 1.26 | 27.46 | 2.88 | 59.40 | 6.87 | 0.49 | 0.25 | 0.00 | 2.05 |
| pCHK1227-5 | 0.03 | 1.26 | 27.24 | 2.85 | 59.45 | 7.10 | 0.49 | 0.25 | 0.00 | 2.87 |
| pCHK1227-6 | 0.04 | 1.30 | 27.22 | 2.86 | 59.09 | 7.34 | 0.52 | 0.25 | 0.00 | 2.34 |
| pCHK1227-7 | 0.03 | 1.26 | 27.32 | 2.83 | 59.30 | 7.12 | 0.50 | 0.25 | 0.00 | 2.89 |
| CHK22 Control | 0.04 | 1.27 | 27.29 | 2.45 | 59.08 | 7.45 | 0.54 | 0.23 | 0.00 | 1.5 |

### Example 50: Expressing Chlamydomonas sp. UWO_241 (CuwoLPAAT2) in P. moriformis Strain CHK22 Using the PmG3PDH Promoter

CuwoLPAAT2 was introduced into a P. moriformis strain CHK22. The expression construct pCHK1228 contained 5′ and 3′ homology arms to permit its targeted integration into the genome and is shown in Table 104.

TABLE 104
Integrative sequences for the transformation of P. moriformis with pCHK1228
encoding CuwoLPAAT2.

| SEQ ID | |
|---|---|
| NO: | Nucleotide Sequence |
| 80 | ccctcaactgcgacgctgggaaccttctccgggcaggcgatgtgcgtgggtttgcctccttggcacggctctacaccgtcga |
| | gtacgccatgaggcggtgatggctgtgtcggttgccacttcgtccagagacggcaagtcgtccatcctctgcgtgtgtggcg |
| | cgacgctgcagcagtccctctgcagcagatgagcgtgactttggccatttcacgcactcgagtgtacacaatccatttttct |
| | taaagcaaatgactgctgattgaccagatactgtaacgctgatttcgctccagatcgcacagatagcgaccatgttgctgcg |
| | tctgaaaatctggattccgaattcgaccctggcgctccatccatgcaacagatggcgacacttgttacaattcctgtcaccc |
| | atcggcatggagcaggtccacttagattcccgatcacccacgcacatctcgctaatagtcattcgttcgtgtcttcgatcaa |
| | tctcaagtgagtgtgcatggatcttggttgacgatgcggtatgggtttgcgccgctggctgcagggtctgcccaaggcaagc |
| | taacccagctcctctccccgacaatactctcgcaggcaaagccggtcacttgccttccagattgccaataaactcaattatg |
| | gcctctgtcatgccatccatgggtctgatgaatggtcacgctcgtgtcctgaccgtt |
| | 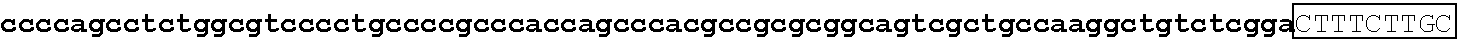 |
| | 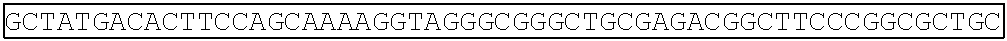 |
| | 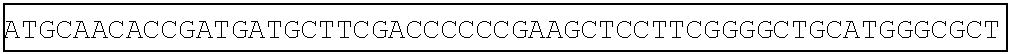 |
| | 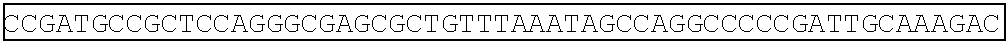 |
| | 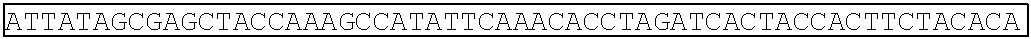 |
| | 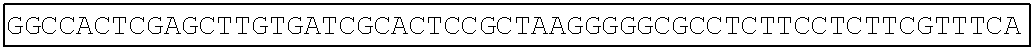 |
| | 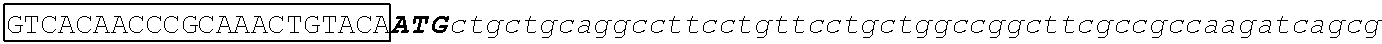 |
| | cctccatgacgaacgagacgtccgaccgccccctggtgcacttcacccccaacaagggctggatgaacgaccccaacggcct |
| | gtggtacgacgagaaggacgccaagtggcacctgtacttccagtacaacccgaacgacaccgtctgggggacgcccttgttc |
| | tggggccacgccacgtccgacgacctgaccaactgggaggaccagcccatcgccatcgccccgaagcgcaacgactccggcg |
| | ccttctccggctccatggtggtggactacaacaacacctccggcttcttcaacgacaccatcgacccgcgccagcgctgcgt |
| | ggccatctggacctacaacaccccggagtccgaggagcagtacatctcctacagcctggacggcggctacaccttcaccgag |
| | taccagaagaaccccgtgctggccgccaactccacccagttccgcgacccgaaggtcttctggtacgagccctcccagaagt |
| | ggatcatgaccgcggccaagtcccaggactacaagatcgagatctactcctccgacgacctgaagtcctggaagctggagtc |
| | cgcgttcgccaacgagggcttcctcggctaccagtacgagtgccccggcctgatcgaggtccccaccgagcaggaccccagc |
| | aagtcctactgggtgatgttcatctccatcaaccccggcgccccggccggcggctccttcaaccagtacttcgtcggcagct |
| | tcaacggcacccacttcgaggccttcgacaaccagtcccgcgtggtggacttcggcaaggactactacgccctgcagacctt |
| | cttcaacaccgacccgacctacgggagcgccctgggcatcgcgtgggcctccaactgggagtactccgccttcgtgcccacc |
| | aacccctggcgctcctccatgtccctcgtgcgcaagttctccctcaacaccgagtaccaggccaacccggagacggagctga |
| | tcaacctgaaggccgagccgatcctgaacatcagcaacgccggcccctggagccggttcgccaccaacaccacgttgacgaa |
| | ggccaacagctacaacgtcgacctgtccaacagcaccggcaccctggagttcgagctggtgtacgccgtcaacaccacccag |
| | acgatctccaagtccgtgttcgcggacctctccctctggttcaagggcctggaggaccccgaggagtacctccgcatgggct |
| | tcgaggtgtccgcgtcctccttcttcctggaccgcgggaacagcaaggtgaagttcgtgaaggagaacccctacttcaccaa |
| | ccgcatgagcgtgaacaaccagcccttcaagagcgagaacgacctgtcctactacaaggtgtacggcttgctggaccagaac |
| | atcctggagctgtacttcaacgacggcgacgtctacatcgacaagttccaggtgcgcgaggtcaagtgactTAAGACGCCCG |
| | CGCGGCGCACCTGACCTGTTCTCTCGAGGGCGCCTGTTCTGCCTTGCGAAACAAGCCCCTGGAGCATGCGTGCATGATCG |
| | TCTCTGGCGCCCCGCCGCGCGGTTTGTCGCCCTCGCGGGCGCCGCGGCCGCGGGGGC |
| | GCATTGAAATTGTTGCAAACCCCACCTGACAGATTGAGGGCCCAGGCAGGAAGGCGT |
| | TGAGATGGAGGTACAGGAGTCAAGTAACTGAAAGTTTTTATGATAACTAACAACAAA |
| | GGGTCGTTTCTGGCCAGCGAATGACAAGAACAAGATTCCACATTTCCGTGTAGAGGC |
| | TTGCCATCGAATGTGAGCGGGCGGGCCGCGGACCCGACAAAACCCTTACGACGTGGT |
| | AAGAAAAACGTGGCGGGCACTGTCCCTGTAGCCTGAAGACCAGCAGGAGACGATCG |
| | GAAGCATCACAGCACAACCGGTgcagcagcagctcggatagtatcgacacactctggacgctggtcgtgtgatggactgttg |
| | ccgccacacttgctgccttgacctgtgaatatccctgccgcttttatcaaacagcctcagtgtgtttgatcttgtgtgtacg |
| | cgcttttgcgagttgctagctgcttgtgctatttgcgaataccacccccagcatccccttccctcgtttcatatcgcttgca |
| | tcccaaccgcaacttatctacgctgtcctgctatccctcagcgctgctcctgctcctgctcactgcccctcgcacagccttg |
| | gtttgggctccgcctgtattctcctggtactgcaacc |
| | 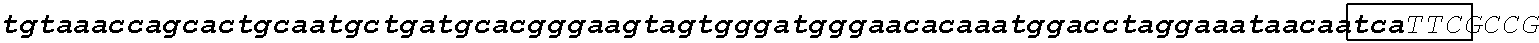 |
| | 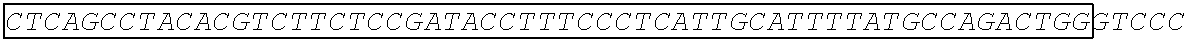 |
| | 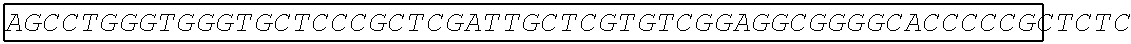 |
| | 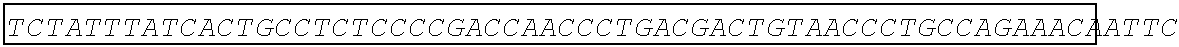 |
| | 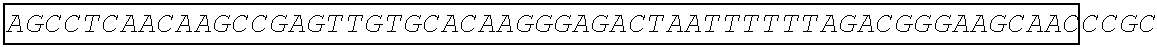 |
| | 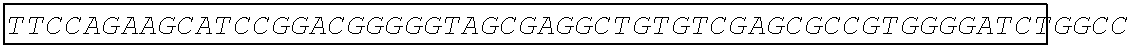 |
| | 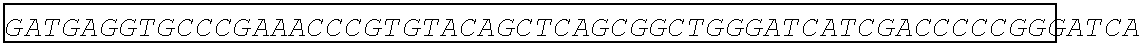 |
| | 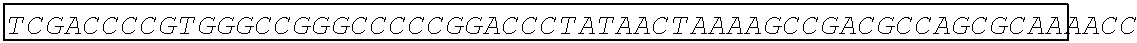 |
| | 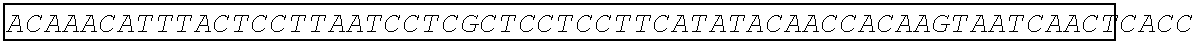 |
| | 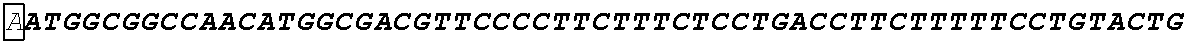 |
| | GTCCATGGCCATCGGCACCCTGGTCGGACGGGTCAAGTTTTGGCAGTACAACGGCACC |
| | AACCGCAACGAGATGTTCGTGTGGGCCAAGCTGTGCTCGGACTTTCTGGGCGTCAAGT |
| | ACCTGAAGACGGGCTCGGGCTCTCTCTACAAGAAGGAGCGCTGCGTGTACCTGAGCAA |
| | CCACCGCTCCTGGGCGGACTTCTTTGTGGACGTGTACCTGACCGAGGGCATCGCGCAG |
| | GCCATGGCGCGAACCATGGTCGGCATCGCCTTCCCCATGTTTATTGGCGCGGCTACCAT |
| | CCTGCGAGGCATCCTCCTGTTTAAGCGGGGCTCCATCGCCAACAAGGACAAGTTCAACA |
| | CCTGGCTCGATGACAAGCTGAACAGCTCCCCGTGCTCCTCTCTGCTCGTGTTTCCCGAG |
| | GGCCACCGAAGCACCAAGTCCACGAGCCTGCCGCTGAAGCGCGGCATGCTGTACTACG |
| | CGCATTCGCGCGGGCTGCCTGTGCAGGTGATCATGACGCGCGGGAAGGAGCTGGTGCT |
| | GTCGGAGAAGCACCATCACGCGGGCTACGGCACCCAACTGATTACGGGCTTTTCCGAG |
| | CTGGTGCTGAGCAAGGATAAGGAGTTCGAGCAGTTTTGGACCGAGGTCCAGGCGATGT |
| | GGGACCGCATGTGGGCCGAGGTGTACAGCGTCGACCCCTCCACGGCGGTGCCTTACAC |
| | GCAGGACGTGCCCGGCAGCGTGTACCCCCTCTCCATGTCGGTCCTGTACACCGTGTTTA |
| | CGGTGTCGACCGTCATCTTTCTGCTCTCGACGCTCTGGATCATGGCGCGGATCTTCGGC |
| | TTCATGATCTCCCCGCTGCCCGCGATGCTCCAAATGGTGGTCTGGGGCGCCGTCCCGGT |
| | CACCCTGGCGGTGTCGTTCCGGGCCTCTATTATCGACAACGCCAAGACGTCCTCGTCCG |
| | CCGTCATCAAGACCGCGAAGAAGGCGCAATGAgtaccacgcccgcgcggcgcacctgacctgttctctcgagg |
| | gcgcctgttctgccttgcgaaacaagcccctggagcatgcgtgcatgatcgtctctggcgccccgccgcgcgg |
| | tttgtcgccctcgcgggcgccccggccgcgggggcgcattgaaattgttgcaaaccccacctgacagattgag |
| | ggcccaggcaggaaggcgttgagatggaggtacaggagtcaagtaactgaaagtttttatgataactaacaac |
| | aaagggtcgtttctggccagcgaatgacaagaacaagattccacatttccgtgtagaggcttgccatcgaatg |
| | tgagcggggggccgcggacccgacaaaacccttacgacgtggtaagaaaaacgtgggggcactgtccctgtag |
| | cctgaagaccagcaggagacgatcggaagcatcacagcacaggccggcccgcgtctcgaacagagcgcggcgg |
| | ccgcatcgatacctgcaggacagcgccatgccacgccctttgatggcttcaagtacgattacggtgttggatt |
| | gtgtgtttgttgcgtagtgtgcatggtttagaataatacacttgatttcttgctcacggcaatctcggcttgt |
| | ccgcaggttcaaccccatttcggagtctcaggtcagccgcgcaatgaccagccgctacttcaaggacttgcac |
| | gacaacgccgaggtgagctatgtttaggacttgattggaaattgtcgtcgacgcatattcgcgctccgcgaca |
| | gcacccaagcaaaatgtcaagtgcgttccgatttgcgtccgcaggtcgatgttgtgatcgtcggcgccggatc |
| | cgccggtctgtcctgcgcttacgagctgaccaagcaccctgacgtccgggtacgcgagctgagattcgattag |
| | acataaattgaagattaaacccgtagaaaaatttgatggtcgcgaaactgtgctcgattgcaagaaattgatc |
| | gtcctccactccgcaggtcgccatcatcgagcagggcgttgctcccggcggcggcgcctggctggggggacag |
| | ctgttctcggccatgtgtgtacgtagaaggatgaatttcagctggttttcgttgcacagctgtttgtgcatga |
| | tttgtttcagactattgttgaatgtttttagatttcttaggatgcatgatttgtctgcatgcgact |

The construct can be written as 5′Thia4::CrTUB2:ScSUC2:PmPGH:CvNR:PmG3PDH:CuwoLPAAT2:PmPGH::3′Thia4. Proceeding in the 5′ to 3′ direction, bold, lowercase sequences represent genomic DNA from CHK22 that permit targeted integration at the Thia4 locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR that enables amplification of the CuwoLPAAT2 gene, is indicated by uppercase underlined text followed by a linker, indicated by lowercase, bold italics, which contains a C. vulgaris nitrate reductase 3′-UTR. The P. moriformis PmG3PDH promoter, indicated by boxed italicized text, drive the expression of the CuwoLPAAT2. The initiator ATG and terminator TGA codons of the CuwoLPAAT2 are indicated by uppercase, bold italics, while the remainder of the gene is indicated by bold italics. The PmPGH 3′-UTR is indicated by lowercase underlined text followed by the CHK22 Thia4 genomic region indicated by bold, lowercase text.

Table 105 shows the fatty acid/TAG profiles of seven primary transformants of CHK22 transformed with pCHK1228. The P. moriformis base strain CHK22 is shown as a non-transgenic control. Strains were grown for 120 hrs in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 105
Screen of primary transformants of CHK22 with pCHK1228.

| | FAME Profiles | | | | | | | | | TAG Profile |
|---|---|---|---|---|---|---|---|---|---|---|
| | | | | | | | C18:3 | | | m/z 577/603 |
| Sample | C12:0 | C14:0 | C16:0 | C18:0 | C18:1 | C18:2 | alpha | C20:0 | C20:1 | (OP:OO) |
| pCHK1228-1 | 0.03 | 1.24 | 27.29 | 2.77 | 59.73 | 6.84 | 0.48 | 0.24 | 0.00 | 2.9 |
| pCHK1228-2 | 0.03 | 1.24 | 27.12 | 2.79 | 59.89 | 6.87 | 0.47 | 0.24 | 0.00 | 3.33 |
| pCHK1228-3 | 0.03 | 1.26 | 27.24 | 2.74 | 59.63 | 7.02 | 0.47 | 0.23 | 0.00 | 3.65 |
| pCHK1228-4 | 0.03 | 1.25 | 26.79 | 2.87 | 60.30 | 6.75 | 0.46 | 0.24 | 0.00 | 3.45 |
| pCHK1228-5 | 0.03 | 1.26 | 27.18 | 2.89 | 59.84 | 6.73 | 0.48 | 0.24 | 0.00 | 2.79 |
| pCHK1228-6 | 0.04 | 1.26 | 27.24 | 2.78 | 59.84 | 6.80 | 0.47 | 0.22 | 0.00 | 3.39 |
| pCHK1228-7 | 0.03 | 1.27 | 27.34 | 2.84 | 59.42 | 6.95 | 0.51 | 0.25 | 0.00 | 3.04 |
| CHK22 Control | 0.04 | 1.27 | 27.29 | 2.45 | 59.08 | 7.45 | 0.54 | 0.23 | 0.00 | 1.5 |

### Example 51: Expressing Chlamydomonas moewusii (CmoeLPAAT2) in P. moriformis Strain CHK22 Using the PmG3PDH Promoter

CmoeLPAAT2 was introduced into a P. moriformis strain CHK22. The expression construct pCHK1231 contained 5′ and 3′ homology arms to permit its targeted integration into the genome and is shown in Table 106.

TABLE 106
Integrative sequences for the transformation of P. moriformis with pCHK1231
encoding CmoeLPAAT2.

| SEQ ID | |
|---|---|
| NO: | Nucleotide Sequence |
| 81 | ccctcaactgcgacgctgggaaccttctccgggcaggcgatgtgcgtgggtttgcctccttggcacggctctacaccgtcga |
| | gtacgccatgaggcggtgatggctgtgtcggttgccacttcgtccagagacggcaagtcgtccatcctctgcgtgtgtggcg |
| | cgacgctgcagcagtccctctgcagcagatgagcgtgactttggccatttcacgcactcgagtgtacacaatccatttttct |
| | taaagcaaatgactgctgattgaccagatactgtaacgctgatttcgctccagatcgcacagatagcgaccatgttgctgcg |
| | tctgaaaatctggattccgaattcgaccctggcgctccatccatgcaacagatggcgacacttgttacaattcctgtcaccc |
| | atcggcatggagcaggtccacttagattcccgatcacccacgcacatctcgctaatagtcattcgttcgtgtcttcgatcaa |
| | tctcaagtgagtgtgcatggatcttggttgacgatgcggtatgggtttgcgccgctggctgcagggtctgcccaaggcaagc |
| | taacccagctcctctccccgacaatactctcgcaggcaaagccggtcacttgccttccagattgccaataaactcaattatg |
| | gcctctgtcatgccatccatgggtctgatgaatggtcacgctcgtgtcctgaccgtt |
| | 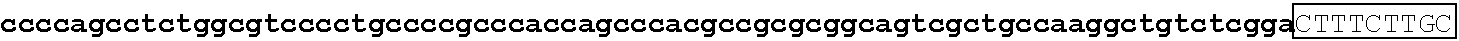 |
| | 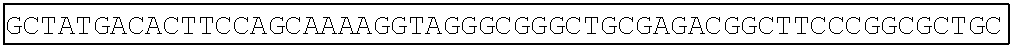 |
| | 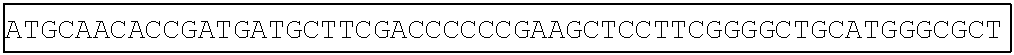 |
| | 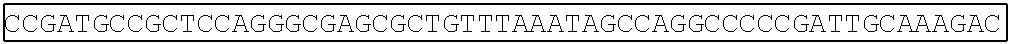 |
| | 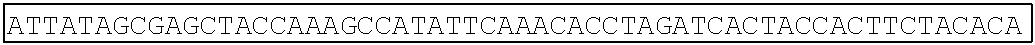 |
| | 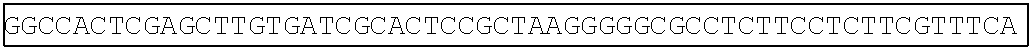 |
| | 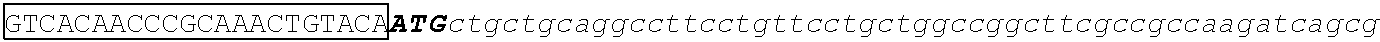 |
| | cctccatgacgaacgagacgtccgaccgccccctggtgcacttcacccccaacaagggctggatgaacgaccccaacggcct |
| | gtggtacgacgagaaggacgccaagtggcacctgtacttccagtacaacccgaacgacaccgtctgggggacgcccttgttc |
| | tggggccacgccacgtccgacgacctgaccaactgggaggaccagcccatcgccatcgccccgaagcgcaacgactccggcg |
| | ccttctccggctccatggtggtggactacaacaacacctccggcttcttcaacgacaccatcgacccgcgccagcgctgcgt |
| | ggccatctggacctacaacaccccggagtccgaggagcagtacatctcctacagcctggacggcggctacaccttcaccgag |
| | taccagaagaaccccgtgctggccgccaactccacccagttccgcgacccgaaggtcttctggtacgagccctcccagaagt |
| | ggatcatgaccgcggccaagtcccaggactacaagatcgagatctactcctccgacgacctgaagtcctggaagctggagtc |
| | cgcgttcgccaacgagggcttcctcggctaccagtacgagtgccccggcctgatcgaggtccccaccgagcaggaccccagc |
| | aagtcctactgggtgatgttcatctccatcaaccccggcgccccggccggcggctccttcaaccagtacttcgtcggcagct |
| | tcaacggcacccacttcgaggccttcgacaaccagtcccgcgtggtggacttcggcaaggactactacgccctgcagacctt |
| | cttcaacaccgacccgacctacgggagcgccctgggcatcgcgtgggcctccaactgggagtactccgccttcgtgcccacc |
| | aacccctggcgctcctccatgtccctcgtgcgcaagttctccctcaacaccgagtaccaggccaacccggagacggagctga |
| | tcaacctgaaggccgagccgatcctgaacatcagcaacgccggcccctggagccggttcgccaccaacaccacgttgacgaa |
| | ggccaacagctacaacgtcgacctgtccaacagcaccggcaccctggagttcgagctggtgtacgccgtcaacaccacccag |
| | acgatctccaagtccgtgttcgcggacctctccctctggttcaagggcctggaggaccccgaggagtacctccgcatgggct |
| | tcgaggtgtccgcgtcctccttcttcctggaccgcgggaacagcaaggtgaagttcgtgaaggagaacccctacttcaccaa |
| | ccgcatgagcgtgaacaaccagcccttcaagagcgagaacgacctgtcctactacaaggtgtacggcttgctggaccagaac |
| | atcctggagctgtacttcaacgacggcgacgtcgtgtccaccaacacctacttcatgaccaccgggaacgccctgggctccg |
| | tgaacatgacgacgggggtggacaacctgttctacatcgacaagttccaggtgcgcgaggtcaagtgactTAAGACGCCCGC |
| | GCGGCGCACCTGACCTGTTCTCTCGAGGGCGCCTGTTCTGCCTTGCGAAACAAGCCCCTGGAGCATGCGTGCATGATCG |
| | TCTCTGGCGCCCCGCCGCGCGGTTTGTCGCCCTCGCGGGCGCCGCGGCCGCGGGGGC |
| | GCATTGAAATTGTTGCAAACCCCACCTGACAGATTGAGGGCCCAGGCAGGAAGGCGT |
| | TGAGATGGAGGTACAGGAGTCAAGTAACTGAAAGTTTTTATGATAACTAACAACAAA |
| | GGGTCGTTTCTGGCCAGCGAATGACAAGAACAAGATTCCACATTTCCGTGTAGAGGC |
| | TTGCCATCGAATGTGAGCGGGGGGCCGCGGACCCGACAAAACCCTTACGACGTGGT |
| | AAGAAAAACGTGGCGGGCACTGTCCCTGTAGCCTGAAGACCAGCAGGAGACGATCG |
| | GAAGCATCACAGCACAACCGGTgcagcagcagctcggatagtatcgacacactctggacgctggtcgtgtgatggac |
| | tgttgccgccacacttgctgccttgacctgtgaatatccctgccgcttttatcaaacagcctcagtgtgtttgatcttgtgt |
| | gtacgcgcttttgcgagttgctagctgcttgtgctatttgcgaataccacccccagcatccccttccctcgtttcatatcgc |
| | ttgcatcccaaccgcaacttatctacgctgtcctgctatccctcagcgctgctcctgctcctgctcactgcccctcgcacag |
| | ccttggtttgggctccgcctgtattctcctggtactgcaacc |
| | 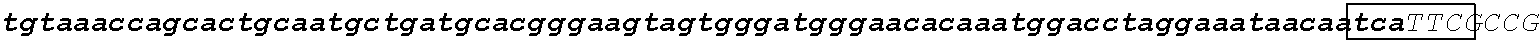 |
| | 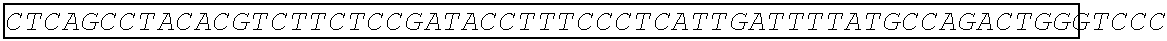 |
| | 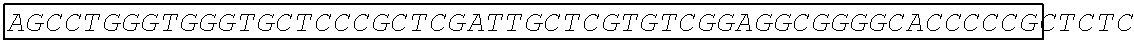 |
| | 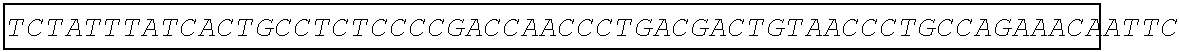 |
| | 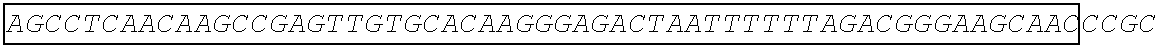 |
| | 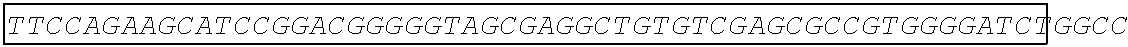 |
| | 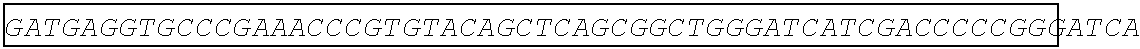 |
| | 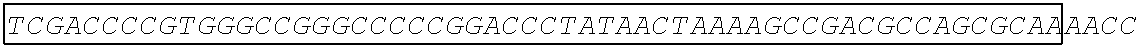 |
| | 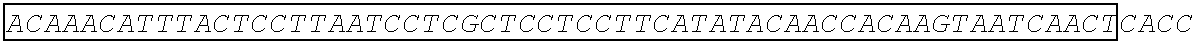 |
| | 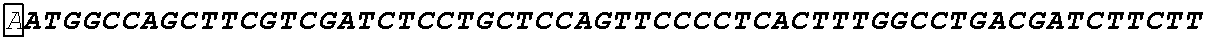 |
| | TTTCTACTGGTCCCCACCCCTGGCGTCCATCGTCTACCGAATCCGCTTCGCGTCGGCGG |
| | GACCCCGCAAGGACATCTACGAGTGGACGACCGCCATGGTCCAGTTCCTCGGCGTGCG |
| | CATCCGTCACCAAGGCAAGCAGACCCTGTACAAGAAGGAGCGCTGCGTCTACCTGTGC |
| | AACCACCGCTCCTGGGCGGACTTTTTCGTGGACCTGCACCTGACCCAAGGCCTGGCGG |
| | CCCCCATGAGCCGCACCATGGTGTTTTTCGCCTTCCCTGTGTTCATGAGCATTTGCATC |
| | GTGCTCAAGGGCGTCCTCCTCTTCAAGCGCGGCACGATCGCCGACAAGACGAAGTTCA |
| | ACAAGTGGCTGGATGACAGCCTGGGCAACAGCCCGGTGTCCTGCCTGCTCGTGTACCC |
| | CGAGGGCCACCGCTCTACGAAGGCGAAGTCGCTGCCGCTGAAGCGCGGTATGCTGTAC |
| | TACGCGCACAGCCGCGGCCTGCCCGTGCAGATTATCCATACCGCGGGCAAGGAGGCCG |
| | TCCTGTCCGAGAAGCGCATGCGCGCGAGCTACGGCCAGACCCTCGTCACGGGCTTCTC |
| | GCAGCCCATCCAGTCCAAGGACTTCAAGGACTTCGAGGCCTTCTCTGCGGAGGTCCAG |
| | CGGAGCTGGGACGCGCTCTGGACCGAGGTGTACTCCGCGAACCCCTCCGCGCTGCCCC |
| | CGCTGGTGCAGGAGGACGTCCCCACCGCGGACTACCCGCCTTCCATGCGCACGGGCAT |
| | GCTCCTGGTCAGCCTGGCGGCCATCGCCCTGTTCCTGGCCTTTGTCGCGCTCCTGGCCT |
| | ACATCACGGGCCCGGTGATGAGCCTGTTCCCCGCTCCCCTCCGCGTCCCACTCATTCTC |
| | TTTAAGCTGTCCTGGCACGCGGCCAGCTTCCTGTACTGCCGGGCCAAGGACACCCCTG |
| | GCAAGGTGAAGGCGGCGTGAgtaccacgcccgcgcggcgcacctgacctgttctctcgagggcgcctgttctgccttgcg |
| | aaacaagcccctggagcatgcgtgcatgatcgtctctggcgccccgccgcgcggtttgtcgccctcgcgggcgccccggccg |
| | cgggggcgcattgaaattgttgcaaaccccacctgacagattgagggcccaggcaggaaggcgttgagatggaggtacagga |
| | gtcaagtaactgaaagtttttatgataactaacaacaaagggtcgtttctggccagcgaatgacaagaacaagattccacat |
| | ttccgtgtagaggcttgccatcgaatgtgagcgggcgggccgcggacccgacaaaacccttacgacgtggtaagaaaaacgt |
| | ggcgggcactgtccctgtagcctgaagaccagcaggagacgatcggaagcatcacagcacaggccggcccgcgtctcgaaca |
| | gagcgcggcggccgcatcgatacctgcaggacagcgccatgccacgccctttgatggcttcaagtacgattacggtgttgga |
| | ttgtgtgtttgttgcgtagtgtgcatggtttagaataatacacttgatttcttgctcacggcaatctcggcttgtccgcagg |
| | ttcaaccccatttcggagtctcaggtcagccgcgcaatgaccagccgctacttcaaggacttgcacgacaacgccgaggtga |
| | gctatgtttaggacttgattggaaattgtcgtcgacgcatattcgcgctccgcgacagcacccaagcaaaatgtcaagtgcg |
| | ttccgatttgcgtccgcaggtcgatgttgtgatcgtcggcgccggatccgccggtctgtcctgcgcttacgagctgaccaag |
| | caccctgacgtccgggtacgcgagctgagattcgattagacataaattgaagattaaacccgtagaaaaatttgatggtcgc |
| | gaaactgtgctcgattgcaagaaattgatcgtcctccactccgcaggtcgccatcatcgagcagggcgttgctcccggcggc |
| | ggcgcctggctggggggacagctgttctcggccatgtgtgtacgtagaaggatgaatttcagctggttttcgttgcacagct |
| | gtttgtgcatgatttgtttcagactattgttgaatgtttttagatttcttaggatgcatgatttgtctgcatgcgact |

The construct can be written as 5′ Thia4::CrTUB2:ScSUC2:PmPGH:CvNR:PmG3PDH:CmoeLPAAT2:PmPGH::3′Thia4. Proceeding in the 5′ to 3′ direction, bold, lowercase sequences represent genomic DNA from CHK22 that permit targeted integration at the Thia4 locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR that enables amplification of the CmoeLPAAT2 gene, is indicated by uppercase underlined text followed by a linker, indicated by lowercase, bold italics, which contains a C. vulgaris nitrate reductase 3′-UTR. The P. moriformis PmG3PDH promoter, indicated by boxed italicized text, drive the expression of the CmoeLPAAT2. The initiator ATG and terminator TGA codons of the CmoeLPAAT2 are indicated by uppercase, bold italics, while the remainder of the gene is indicated by bold italics. The PmPGH 3′-UTR is indicated by lowercase underlined text followed by the CHK22 Thia4 genomic region indicated by bold, lowercase text.

Table 107 shows the fatty acid/TAG profiles of seven primary transformants of CHK22 transformed with pCHK1231. The P. moriformis base strain CHK22 is shown as a non-transgenic control. Strains were grown for 120 hrs in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 107
Screen of primary transformants of CHK22 with pCHK1231.

| | FAME Profiles | | | | | | | | | TAG Profile |
|---|---|---|---|---|---|---|---|---|---|---|
| | | | | | | | C18:3 | | | m/z 577/603 |
| Sample | C12:0 | C14:0 | C16:0 | C18:0 | C18:1 | C18:2 | alpha | C20:0 | C20:1 | (OP:OO) |
| pCHK1231-1 | 0.03 | 1.19 | 26.09 | 3.30 | 60.84 | 6.59 | 0.52 | 0.31 | 0.00 | 1.55 |
| pCHK1231-2 | 0.04 | 1.29 | 21.15 | 4.13 | 61.84 | 9.50 | 0.58 | 0.41 | 0.00 | 1.52 |
| pCHK1231-3 | 0.03 | 1.34 | 26.69 | 2.91 | 58.84 | 8.26 | 0.50 | 0.24 | 0.00 | 4.45 |
| pCHK1231-4 | 0.03 | 1.33 | 26.34 | 3.43 | 59.14 | 7.80 | 0.52 | 0.28 | 0.00 | 4.61 |
| pCHK1231-5 | 0.03 | 1.24 | 26.27 | 3.30 | 60.38 | 6.81 | 0.55 | 0.28 | 0.00 | 1.53 |
| pCHK1231-6 | 0.03 | 1.36 | 26.40 | 2.20 | 60.87 | 7.26 | 0.31 | 0.16 | 0.00 | 4.49 |
| pCHK1231-7 | 0.03 | 1.27 | 26.59 | 3.12 | 59.06 | 7.91 | 0.53 | 0.27 | 0.00 | 4.61 |
| pCHK1231-8 | 0.03 | 1.27 | 26.39 | 2.97 | 59.77 | 7.68 | 0.49 | 0.21 | 0.00 | 4.79 |
| CHK22 Control | 0.04 | 1.4 | 25.8 | 2.69 | 61.83 | 6.51 | 0.36 | 0.16 | 0 | 1.5 |

### Example 52: Expressing Oophila amblystomatis (OambLPAAT2) in P. moriformis Strain CHK22 Using the PmG3PDH Promoter

OambLPAAT2 was introduced into a P. moriformis strain CHK22. The expression construct pCHK1232 contained 5′ and 3′ homology arms to permit its targeted integration into the genome and is shown in Table 108.

TABLE 108
Integrative sequences for the transformation of P. moriformis with pCHK1232
encoding OambLPAAT2.

| SEQ ID | |
|---|---|
| NO: | Nucleotide Sequence |
| 82 | ccctcaactgcgacgctgggaaccttctccgggcaggcgatgtgcgtgggtttgcctccttggcacggctctacaccgtcga |
| | gtacgccatgaggcggtgatggctgtgtcggttgccacttcgtccagagacggcaagtcgtccatcctctgcgtgtgtggcg |
| | cgacgctgcagcagtccctctgcagcagatgagcgtgactttggccatttcacgcactcgagtgtacacaatccatttttct |
| | taaagcaaatgactgctgattgaccagatactgtaacgctgatttcgctccagatcgcacagatagcgaccatgttgctgcg |
| | tctgaaaatctggattccgaattcgaccctggcgctccatccatgcaacagatggcgacacttgttacaattcctgtcaccc |
| | atcggcatggagcaggtccacttagattcccgatcacccacgcacatctcgctaatagtcattcgttcgtgtcttcgatcaa |
| | tctcaagtgagtgtgcatggatcttggttgacgatgcggtatgggtttgcgccgctggctgcagggtctgcccaaggcaagc |
| | taacccagctcctctccccgacaatactctcgcaggcaaagccggtcacttgccttccagattgccaataaactcaattatg |
| | gcctctgtcatgccatccatgggtctgatgaatggtcacgctcgtgtcctgaccgtt |
| | 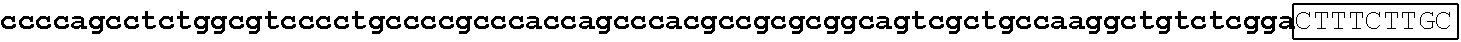 |
| | 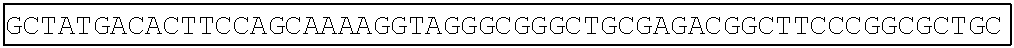 |
| | 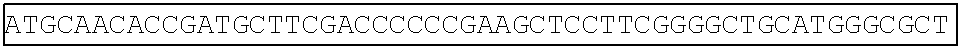 |
| | 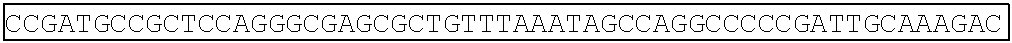 |
| | 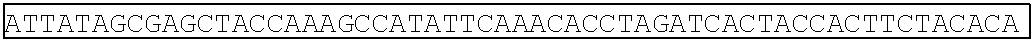 |
| | 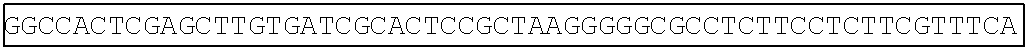 |
| | 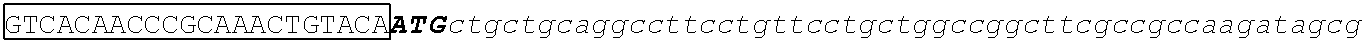 |
| | cctccatgacgaacgagacgtccgaccgccccctggtgcacttcacccccaacaagggctggatgaacgaccccaacggcct |
| | gtggtacgacgagaaggacgccaagtggcacctgtacttccagtacaacccgaacgacaccgtctgggggacgcccttgttc |
| | tggggccacgccacgtccgacgacctgaccaactgggaggaccagcccatcgccatcgccccgaagcgcaacgactccggcg |
| | ccttctccggctccatggtggtggactacaacaacacctccggcttcttcaacgacaccatcgacccgcgccagcgctgcgt |
| | ggccatctggacctacaacaccccggagtccgaggagcagtacatctcctacagcctggacggcggctacaccttcaccgag |
| | taccagaagaaccccgtgctggccgccaactccacccagttccgcgacccgaaggtcttctggtacgagccctcccagaagt |
| | ggatcatgaccgcggccaagtcccaggactacaagatcgagatctactcctccgacgacctgaagtcctggaagctggagtc |
| | cgcgttcgccaacgagggcttcctcggctaccagtacgagtgccccggcctgatcgaggtccccaccgagcaggaccccagc |
| | aagtcctactgggtgatgttcatctccatcaaccccggcgccccggccggcggctccttcaaccagtacttcgtcggcagct |
| | tcaacggcacccacttcgaggccttcgacaaccagtcccgcgtggtggacttcggcaaggactactacgccctgcagacctt |
| | cttcaacaccgacccgacctacgggagcgccctgggcatcgcgtgggcctccaactgggagtactccgccttcgtgcccacc |
| | aacccctggcgctcctccatgtccctcgtgcgcaagttctccctcaacaccgagtaccaggccaacccggagacggagctga |
| | tcaacctgaaggccgagccgatcctgaacatcagcaacgccggcccctggagccggttcgccaccaacaccacgttgacgaa |
| | ggccaacagctacaacgtcgacctgtccaacagcaccggcaccctggagttcgagctggtgtacgccgtcaacaccacccag |
| | acgatctccaagtccgtgttcgcggacctctccctctggttcaagggcctggaggaccccgaggagtacctccgcatgggct |
| | tcgaggtgtccgcgtcctccttcttcctggaccgcgggaacagcaaggtgaagttcgtgaaggagaacccctacttcaccaa |
| | ccgcatgagcgtgaacaaccagcccttcaagagcgagaacgacctgtcctactacaaggtgtacggcttgctggaccagaac |
| | atcctggagctgtacttcaacgacggcgacgtcgtgtccaccaacacctacttcatgaccaccgggaacgccctgggctccg |
| | tgaacatgacgacgggggtggacaacctgttctacatcgacaagttccaggtgcgcgaggtcaagtgactTAAGACGCCCGC |
| | GCGGCGCACCTGACCTGTTCTCTCGAGGGCGCCTGTTCTGCCTTGCGAAACAAGCCCCTGGAGCATGCGTGCATGATCG |
| | TCTCTGGCGCCCCGCCGCGCGGTTTGTCGCCCTCGCGGGCGCCGCGGCCGCGGGGGC |
| | GCATTGAAATTGTTGCAAACCCCACCTGACAGATTGAGGGCCCAGGCAGGAAGGCGT |
| | TGAGATGGAGGTACAGGAGTCAAGTAACTGAAAGTTTTTATGATAACTAACAACAAA |
| | GGGTCGTTTCTGGCCAGCGAATGACAAGAACAAGATTCCACATTTCCGTGTAGAGGC |
| | TTGCCATCGAATGTGAGCGGGCGGGCCGCGGACCCGACAAAACCCTTACGACGTGGT |
| | AAGAAAAACGTGGCGGGCACTGTCCCTGTAGCCTGAAGACCAGCAGGAGACGATCG |
| | GAAGCATCACAGCACAACCGGTgcagcagcagctcggatagtatcgacacactctggacgctggtcgtgtgatggac |
| | tgttgccgccacacttgctgccttgacctgtgaatatccctgccgcttttatcaaacagcctcagtgtgtttgatcttgtgt |
| | gtacgcgcttttgcgagttgctagctgcttgtgctatttgcgaataccacccccagcatccccttccctcgtttcatatcgc |
| | ttgcatcccaaccgcaacttatctacgctgtcctgctatccctcagcgctgctcctgctcctgctcactgcccctcgcacag |
| | ccttggtttgggctccgcctgtattctcctggtactgcaacc |
| | 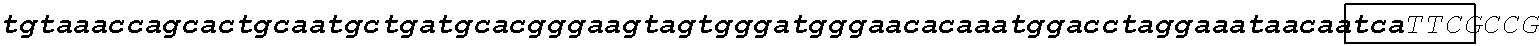 |
| | 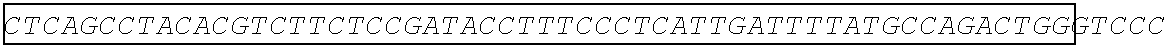 |
| | 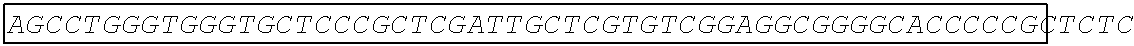 |
| | 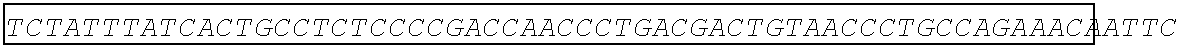 |
| | 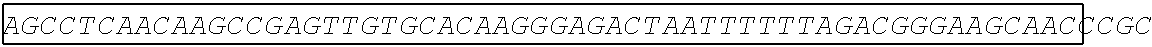 |
| | 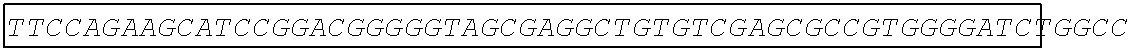 |
| | 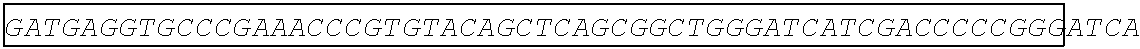 |
| | 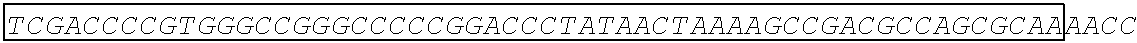 |
| | 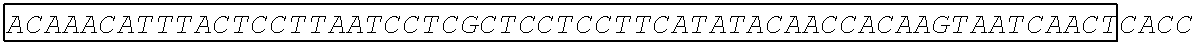 |
| | 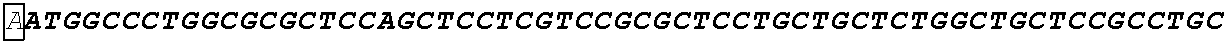 |
| | CGTACTTTCTGCTCACCATCACCTTTTTCTACTGGTCCGCCACGATCGGCGCCCTGATCT |
| | ACCGCGTCCGCTTCCTGTCTACCGGGCCGCGCAACGACATGTACGACTGGACCCTGCTC |
| | ATGACCTCGTTCCTGGGTCTCCACTTCCACCAACTGGGTGCCCAACGCCTGCACTGCGC |
| | GCCCCGAGTCGTGTTTCTGGCCAACCACCGAAGCTGGGCCGACTTTTTCGTCGACATCC |
| | ACCTGACCCAGGGGACCGCCGCTCCCATGTCCCGCGCGCTGGTGGCTTTTGCGTTTCCT |
| | GTGTTCATGTGCACCGCCTGGCTCCTGCGCGGACTGATTATCTTCAAGCGCGGCGCGAT |
| | CGAGGACAAAAAGAAGTTCAACTCGTGGCTGGACGAGCAGCTCGACGCGTCCGCGGGC |
| | TCGAACCTCCTGGTGTACCCCGAGGGACACCGCAGCACCCGACCCACCTCCCTCCCACT |
| | GAAGCGCGGCATGCTGTACTACGCCCACTCCCGCGGCCTGCCCGTGCAAATTATCATGA |
| | CCGCTGGCAAGGAGTCTGTCCTGTCGGAGAAGCACCAGTGCGCGTCCTTTGGCCAACA |
| | CCTCCTGACCAGCTTCAGCCCCGTGATCTCGTCTAAGGACAAGGACTTCGAGGTGTTCG |
| | CTACGGAGGTCCAGCAACGCTGGGATTCGCTGTGGCGCGAGGTGTTTAGCACGTCGAC |
| | CCAAGGCGTCCCCCTCCTGCACCAGGCCAGCGTCCCACTCTTCGACTACCCTCCCGCTG |
| | TCCGCACGCACATGGCTCTCGCTTCCAGCGCCTTCATCCTGATGCTGCTCCTGGCCGCG |
| | GGCGCGGCCGTCCTGGCCGTCCGCGCTACCCTGGCCCTGATGCCGCCCGTCCTCCAGG |
| | TCGTGGTCCTCGCTGCCAGCTCCCTCCTGATGCTCGCGTCCTTTCTGACGTCGGCTGCC |
| | ATCACCTCGTCCACCACGACCACGACCACGACCACGCGACAGCCCCAGGCTGTGGCCG |
| | ACAAGGGCTCGAAGGAGTCCTAGgtaccacgcccgcgcggcgcacctgacctgttctctcgaggggcctgttctgcct |
| | tgcgaaacaagcccctggagcatgcgtgcatgatcgtctctggcgccccgccgcgcggtttgtcgccctcgcgggcgccccg |
| | gccgcgggggcgcattgaaattgttgcaaaccccacctgacagattgagggcccaggcaggaaggcgttgagatggaggtac |
| | aggagtcaagtaactgaaagtttttatgataactaacaacaaagggtcgtttctggccagcgaatgacaagaacaagattcc |
| | acatttccgtgtagaggcttgccatogaatgtgagcgggcgggccgcggacccgacaaaacccttacgacgtggtaagaaaa |
| | acgtgggggcactgtccctgtagcctgaagaccagcaggagacgatcggaagcatcacagcacaggccggcccgcgtctcga |
| | acagagcgcggggccgcatcgatacctgcaggacagcgccatgccacgccctttgatggcttcaagtacgattacggtgttg |
| | gattgtgtgtttgttgcgtagtgtgcatggtttagaataatacacttgatttcttgctcacggcaatctcggcttgtccgca |
| | ggttcaaccccatttcggagtctcaggtcagccgcgcaatgaccagccgctacttcaaggacttgcacgacaacgccgaggt |
| | gagctatgtttaggacttgattggaaattgtcgtcgacgcatattcgcgctccgcgacagcacccaagcaaaatgtcaagtg |
| | cgttccgatttgcgtccgcaggtcgatgttgtgatcgtcggcgccggatccgccggtctgtcctgcgcttacgagctgacca |
| | agcaccctgacgtccgggtacgcgagctgagattcgattagacataaattgaagattaaacccgtagaaaaatttgatggtc |
| | gcgaaactgtgctcgattgcaagaaattgatcgtcctccactccgcaggtcgccatcatcgagcagggcgttgctcccggcg |
| | gcggcgcctggctggggggacagctgttctcggccatgtgtgtacgtagaaggatgaatttcagctggttttcgttgcacag |
| | ctgtttgtgcatgatttgtttcagactattgttgaatgtttttagatttcttaggatgcatgatttgtctgcatgcgact |

The construct can be written as 5′Thia4::CrTUB2:ScSUC2:PmPGH:CvNR:PmG3PDH:OambLPAAT2:PmPGH::3′Thia4. Proceeding in the 5′ to 3′ direction, bold, lowercase sequences represent genomic DNA from CHK22 that permit targeted integration at the Thia4 locus via homologous recombination, the C. reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR that enables amplification of the OambLPAAT2 gene, is indicated by uppercase underlined text followed by a linker, indicated by lowercase, bold italics, which contains a C. vulgaris nitrate reductase 3′-UTR. The P. moriformis PmG3PDH promoter, indicated by boxed italicized text, drive the expression of the OambLPAAT2. The initiator ATG and terminator TAG codons of the OambLPAAT2 are indicated by uppercase, bold italics, while the remainder of the gene is indicated by bold italics. The PmPGH 3′-UTR is indicated by lowercase underlined text followed by the CHK22 Thia4 genomic region indicated by bold, lowercase text.

Table 109 shows the fatty acid/TAG profiles of seven primary transformants of CHK22 transformed with pCHK1232. The P. moriformis base strain CHK22 is shown as a non-transgenic control. Strains were grown for 120 hrs in 50-mL conical tube at 200 rpm at 28° C. in lipid production medium. Biomass was harvested, lyophilized to dryness, and subjected to direct transesterification to generate FAMEs for subsequent quantitation and characterization by GC/FID. TAG analysis was conducted using the LCMS method as described above.

TABLE 109
Screen of primary transformants of CHK22 with pCHK1232.

| | FAME Profiles | | | | | | | | | TAG Profile |
|---|---|---|---|---|---|---|---|---|---|---|
| | | | | | | | C18:3 | | | m/z 577/603 |
| Sample | C12:0 | C14:0 | C16:0 | C18:0 | C18:1 | C18:2 | alpha | C20:0 | C20:1 | (OP:OO) |
| pCHK1232-1 | 0.15 | 3.52 | 26.66 | 2.19 | 48.87 | 15.20 | 1.38 | 0.28 | 0.00 | 2.45 |
| pCHK1232-2 | 0.08 | 2.61 | 27.32 | 2.45 | 58.12 | 7.55 | 0.34 | 0.21 | 0.00 | 1.86 |
| pCHK1232-3 | 0.07 | 2.31 | 27.85 | 4.06 | 57.08 | 6.64 | 0.59 | 0.29 | 0.00 | 1.54 |
| pCHK1232-4 | 0.04 | 1.37 | 25.80 | 3.54 | 60.00 | 7.29 | 0.55 | 0.29 | 0.00 | 2.31 |
| pCHK1232-5 | 0.05 | 1.67 | 27.41 | 2.92 | 58.02 | 8.15 | 0.40 | 0.23 | 0.00 | 1.99 |
| pCHK1232-6 | 0.03 | 1.27 | 26.15 | 3.51 | 60.39 | 6.87 | 0.52 | 0.25 | 0.00 | 2.74 |
| pCHK1232-7 | 0.04 | 1.32 | 27.26 | 2.73 | 55.53 | 10.51 | 0.76 | 0.37 | 0.00 | 1.54 |
| pCHK1232-8 | 0.07 | 1.97 | 25.97 | 3.09 | 55.03 | 11.16 | 0.91 | 0.38 | 0.00 | 2.09 |
| CHK22 Control | 0.04 | 1.4 | 25.8 | 2.69 | 61.83 | 6.51 | 0.36 | 0.16 | 0 | 1.5 |

The amino acid sequences of various LPAATs disclosed herein were compared with the amino acid sequence of CrLPAAT2 to generate a sequence percent identity (%). The results are shown in Table 110. The resulting phenotypes of expressing these LPAATs in the non-transgenic cell line CHK22 is indicated with the OP:OO ratio.

In some embodiments, a microalgal cell provided herein comprises an exogenous gene that encodes for a LPAAT in Table 110. In some embodiments, the exogenous gene can comprise a sequence with at least 50%, at least 60%, at least 70%, at least 80%, at least 90%, at least 95%, at least 96%, at least 97%, at least 98%, at least 99%, or 100% sequence identity to a LPAAT in Table 110.

TABLE 110
Percent identity against CrLPAAT2 and OP:OO Ratio

| | | GenBank | | | TAG profile |
|---|---|---|---|---|---|
| | | ID/Accession | Protein | Identity | m/z (577/603) |
| Gene name | Organism | Number | size | (%) | OP:OO |
| CrLPAAT2 | Chlamydomonas reinhardtii | XP_001699586.2 | 325 aa | 100.0 | 3.45 |
| CiLPAAT2 | Chlamydomonas incerta | KAG2426747.1 | 325 aa | 89.8 | 2.21 |
| VrLPAAT2 | Volvox reticuliferus | GIM16764.1 | 156 aa | 80.8 | 1.69 |
| ChsLPAAT2 | Chlamydomonas schloesseri | KAG2454274.1 | 378 aa | 77.2 | 1.99 |
| EdLPAAT2 | Edaphochlamys | SRR13719274 | 324 aa | 76.9 | 1.8 |
| | debaryana_SRR13719274 | | | | |
| VitrLPAAT2 | Vitreochlamys sp. CL- | SRR13719283 | 328 aa | 70.2 | 4.3 |
| | 2021_SRR13719283 | | | | |
| CchaLPAAT2 | Colemanosphaera | SRR13719279 | 324 aa | 69.2 | 2.6 |
| | charkowiensis_SRR13719279 | | | | |
| VaLPAAT2 | Volvox africanus | GIL63906.1 | 324 aa | 69.2 | 1.96 |
| AgLPAAT2 | Astrephomene gubernaculifera | GFR47784.1 | 354 aa | 69.1 | 2.21 |
| GvirLPAAT2 | Gonium | SRR13719236 | 326 aa | 68.8 | 3.6 |
| | viridistellatum_SRR13719236 | | | | |
| VaulLPAAT2 | Vitreochlamys | SRR13719273 | 325 aa | 68.7 | 4.6 |
| | aulata_SRR13719273 | | | | |
| GmulLPAAT2 | Gonium | SRR13719234 | 324 aa | 68.6 | 4.2 |
| | multicoccum_SRR13719234 | | | | |
| VcomLPAAT2 | Volvulina | SRR888100 | 326 aa | 68.5 | 4.4 |
| | compacta_SRR888100 | | | | |
| PjapLPAAT2 | Pleodorina | SRR1371926 | 324 aa | 67.7 | 2 |
| | japonica_SRR13719268 | | | | |
| PmorLPAAT2 | Pandorina | SRR13719246 | 325 aa | 67.2 | 2 |
| | morum_SRR13719246 | | | | |
| VbolLPAAT2 | Volvulina boldii_SRR13719245 | SRR13719245 | 325 aa | 66.9 | 2.1 |
| EcylLPAAT2 | Eudorina | SRR13719256 | 325 aa | 66.3 | 2 |
| | cylindrica_SRR13719256 | | | | |
| VcarfLPAAT2 | Volvox carteri f. | SRR13719248 | 326 aa | 65.4 | 3.4 |
| | weismannia_SRR13719248 | | | | |
| VferLPAAT2 | Volvox ferrisii_SRR13719278 | SRR13719278 | 325 aa | 65.4 | 3.8 |
| GoctLPAAT2 | Gonium | SRR13719233 | 351 aa | 65.2 | 1.63 |
| | octonarium_SRR13719233 | | | | |
| VsteLPAAT2 | Volvulina steinii_SRR13719272 | SRR13719272 | 326 aa | 63.3 | 1.92 |
| EdLPAAT2 | Edaphochlamys debaryana | KAG2489453.1 | 325 aa | 60.1 | 3.78 |
| CeLPAAT2 | Chlamydomonas eustigma | GAX78532.1 | 253 aa | 55.3 | 1.59 |
| Ch_CCAC2762_{—} | Chlamydomonas sp. | ERR3487411 | 386 aa | 48.6 | 4.59 |
| LPAAT2 | CCAC2762_B_ERR3487411 | | | | |
| VcLPAAT2 | Volvox carteri_98847 | XP_002957774.1 | 303 aa | 47.5 | 1.58 |
| CnivLPAAT2 | Chlamydomonas | SRR12517273 | 360 aa | 45.8 | 1.59 |
| | nivalis_SRR12517273 | | | | |
| CmoeLPAAT2 | Chlamydomonas_moewusii_{—} | SRR13719241 | 336 aa | 45.4 | 4.79 |
| | SRR13719241 | | | | |
| PedLPAAT2 | Pedinophyceae sp. YPF-701 | CAG9465508.1 | 340 aa | 45.4 | 4.47 |
| MyarcLPAAT2 | Microglena | SRR18294403 | 362 aa | 45.1 | 2.87 |
| | sp. YARC_SRR18294403 | | | | |
| DsalLPAAT2 | Dunaliella salina_SRR9129444 | SRR9129444 | 335 aa | 44.5 | 2.06 |
| DsLPAAT2 | Dunaliella salina | KAF5838861.1 | 333 aa | 44.2 | 2.35 |
| VcLPAAT2 | Volvox carteri f. | XP_002947580.1 | 624 aa | 43.4 | 1.59 |
| | nagariensis_120491 | | | | |
| OambLPAAT2 | Oophila | SRR5445907 | 359 aa | 42.4 | 2.74 |
| | amblystomatis_SRR5445907 | | | | |
| ChdeLPAAT2 | Chlorella desiccata | KAH7617329.1 | 372 aa | 41.5 | 1.71 |
| SceLPAAT2 | Scenedesmus sp. NREL 46B-D3 | KAF6259536.1 | 358 aa | 41.4 | 1.64 |
| ChsoLPAAT2 | Chlorella sorokiniana | PRW57325.1 | 363 aa | 41.3 | 1.53 |
| CuwoLPAAT2 | Chlamydomonas_sp. UWO_241_{—} | SRR13696614 | 341 aa | 40.6 | 3.65 |
| | SRR13696614 | | | | |
| McLPAAT2 | Micractinium conductrix | PSC72189.1 | 426 aa | 40.3 | 1.55 |
| RsLPAAT2 | Raphidocelis subcapitata | GBF93205.1 | 380 aa | 39.8 | 1.58 |
| ChvaLPAAT2 | Chlorella variabilis | XP_005852243.1 | 357 aa | 39.8 | 1.58 |
| ChvuLPAAT2 | Chlorella vulgaris | KAI3438340.1 | 360 aa | 39.5 | 1.63 |
| ApLPAAT2 | Auxenochlorella protothecoides | XP_011401335.1 | 385 aa | 37.0 | 1.58 |
| ChprLPAAT2 | Chloropicon primus | QDZ21586.1 | 335 aa | 32.9 | 1.62 |
| CosLPAAT2 | Coccomyxa subellipsoidea | XP_005647638.1 | 197 aa | 30.2 | 1.63 |
| BpraLPAAT2 | Bathycoccus | ERR2041002 | 334 aa | 29.1 | 1.66 |
| | prasinos_ERR2041002 | | | | |
| OlLPAAT2 | Ostreococcus lucimarinus | XP_001421357.1 | 286 aa | 26.8 | 1.63 |
| PproLPAAT2 | Pycnococcus provasolii_{—} | ERR2041000 | 326aa | 22.7 | 1.64 |
| | ERR2041000 | | | | |
| NoLPAT4 | Nannochloropsis oceanica | BAW99690.1 | 445 aa | 15.3 | 1.55 |
| CrLPAAT1 | Chlamydomonas reinhardtii | XP_042921325.1 | 332 aa | 13.6 | 1.59 |
| CrLPAAT1- | Chlamydomonas reinhardtii | XP_042921325.1 | 302aa | 13.6 | 1.69 |
| ΔCST* | | | | | |
| Sll1848 | Synechocystis sp. PCC6803 | WP_010873224.1 | 225 aa | 13.3 | 1.6 |
| EcplsC | Escherichia coli | HBN6599230.1 | 245 aa | 12.4 | 1.59 |
| AGPAT1 | Homo sapiens | NP_001358366.1 | 283 aa | 12.1 | 3.6 |
| BnBAT2- | Brassica napus | NP_001302928.1 | 258 aa | 11.9 | 1.9 |
| ΔCST* | | | | | |
| NoLPAT3 | Nannochloropsis oceanica | BAW99691.1 | 482 aa | 11.1 | 1.6 |
| BnBAT2 | Brassica napus | NP_001302928.1 | 344 aa | 10.3 | 1.57 |

The amino acid sequences were compared with the amino acid sequence of PedLPAAT2 to generate a sequence percent identity (%). The results are shown in Table 111. The resulted phenotype of expressing these LPAATs in CHK22 is indicated with the OP:00 ratio.

In some embodiments, a microalgal cell provided herein comprises an exogenous gene that encodes for a LPAAT in Table 111. In some embodiments, the exogenous gene can comprise a sequence with at least 50%, at least 60%, at least 70%, at least 80%, at least 90%, at least 95%, at least 96%, at least 97%, at least 98%, at least 99%, or 100% sequence identity to a LPAAT in Table 111.

TABLE 111
Percent identity against PedLPAAT2 and OP:OO Ratio

| | | GenBank | | | TAG profile m/z |
|---|---|---|---|---|---|
| | | ID/Accession | Protein | Identity | (577/603) |
| Gene name | Organism | Number | size | (%) | OP:OO |
| PedLPAAT2 | Pedinophyceae sp. YPF-701 | CAG9465508.1 | 340 aa | 100 | 4.47 |
| VcomLPAAT2 | Volvulina | SRR888100 | 326 aa | 45.9 | 4.4 |
| | compacta_SRR888100 | | | | |
| CmoeLPAAT2 | Chlamydomonas | SRR13719241 | 336 aa | 45.9 | 4.79 |
| | moewusii_SRR13719241 | | | | |
| CchaLPAAT2 | Colemanosphaera | SRR13719279 | 324 aa | 43.8 | 2.6 |
| | charkowiensis_SRR13719279 | | | | |
| VaLPAAT2 | Volvox africanus | GIL63906.1 | 324 aa | 43.5 | 1.96 |
| VferLPAAT2 | Volvox | SRR13719278 | 325 aa | 43.5 | 3.8 |
| | ferrisii_SRR13719278 | | | | |
| GvirLPAAT2 | Gonium | SRR13719236 | 326 aa | 43.5 | 3.6 |
| | viridistellatum_SRR13719236 | | | | |
| VcarfLPAAT2 | Volvox carteri f. | SRR13719248 | 326 aa | 43.5 | 3.4 |
| | weismannia_SRR13719248 | | | | |
| VsteLPAAT2 | Volvulina | SRR13719272 | 326 aa | 43.5 | 1.92 |
| | steinii_SRR13719272 | | | | |
| VcLPAAT2 V2 | Volvox carteri f. | XP_002947580.1 | 624 aa | 43.5 | 1.59 |
| | nagariensis_120491 | | | | |
| CrLPAAT2 | Chlamydomonas | XP_001699586.2 | 325 aa | 43.2 | 3.45 |
| | reinhardtii | | | | |
| VbolLPAAT2 | Volvulina | SRR13719245 | 325 aa | 43.2 | 2.1 |
| | boldii_SRR13719245 | | | | |
| AgLPAAT2 | Astrephomene | GFR47784.1 | 354 aa | 42.9 | 2.21 |
| | gubernaculifera | | | | |
| CiLPAAT2 | Chlamydomonas incerta | KAG2426747.1 | 325 aa | 42.9 | 2.21 |
| VitrLPAAT2 | Vitreochlamys sp. CL- | SRR13719283 | 328 aa | 42.9 | 4.3 |
| | 2021_SRR13719283 | | | | |
| Ch_CCAC2762_{—} | Chlamydomonas sp. | ERR3487411 | 386 aa | 42.9 | 4.59 |
| LPAAT2 | CCAC2762_B_ERR3487411 | | | | |
| EdLPAAT2 | Edaphochlamys debaryana | KAG2489453.1 | 325 aa | 42.6 | 3.78 |
| ChsLPAAT2 | Chlamydomonas | KAG2454274.1 | 378 aa | 42.6 | 1.99 |
| | schloesseri | | | | |
| EcylLPAAT2 | Eudorina | SRR13719256 | 325 aa | 42.4 | 2 |
| | cylindrica_SRR13719256 | | | | |
| GoctLPAAT2 | Gonium | SRR13719233 | 351 aa | 42.4 | 1.63 |
| | octonarium_SRR13719233 | | | | |
| PjapLPAAT2 | Pleodorina | SRR1371926 | 324 aa | 42.4 | 2 |
| | japonica_SRR13719268 | | | | |
| GmulLPAAT2 | Gonium | SRR13719234 | 324 aa | 42.1 | 4.2 |
| | multicoccum_SRR13719234 | | | | |
| OambLPAAT2 | Oophila | SRR5445907 | 359 aa | 41.5 | 2.74 |
| | amblystomatis_SRR5445907 | | | | |
| PmorLPAAT2 | Pandorina | SRR13719246 | 325 aa | 41.2 | 2 |
| | morum_SRR13719246 | | | | |
| EdLPAAT2 | Edaphochlamys | SRR13719274 | 324 aa | 40.6 | 1.8 |
| | debaryana_SRR13719274 | | | | |
| VaulLPAAT2 | Vitreochlamys | SRR13719273 | 325 aa | 40.0 | 4.6 |
| | aulata_SRR13719273 | | | | |
| DsLPAAT2 | Dunaliella salina | KAF5838861.1 | 333 aa | 40.0 | 2.35 |
| DsalLPAAT2 | Dunaliella | SRR9129444 | 335 aa | 39.4 | 2.06 |
| | salina_SRR9129444 | | | | |
| CuwoLPAAT2 | Chlamydomonas | SRR13696614 | 342 aa | 39.4 | 3.65 |
| | sp. UWO_241_SRR13696614 | | | | |
| RsLPAAT2 | Raphidocelis subcapitata | GBF93205.1 | 380 aa | 39.4 | 1.58 |
| Ce LPAAT2 | Chlamydomonas eustigma | GAX78532.1 | 253 aa | 38.8 | 1.59 |
| MyarcLPAAT2 | Microglena | SRR18294403 | 362 aa | 38.5 | 2.87 |
| | sp. YARC_SRR18294403 | | | | |
| SceLPAAT2 | Scenedesmus sp. NREL | KAF6259536.1 | 358 aa | 37.1 | 1.64 |
| | 46B-D3 | | | | |
| McLPAAT2 | Micractinium conductrix | PSC72189.1 | 426 aa | 36.8 | 1.55 |
| CnivLPAAT2 | Chlamydomonas | SRR12517273 | 360 aa | 36.2 | 1.59 |
| | nivalis_SRR12517273 | | | | |
| VcLPAAT2 | Volvox carteri_98847 | XP_002957774.1 | 303 aa | 35.9 | 1.58 |
| ChvuLPAAT2 | Chlorella vulgaris | KAI3438340.1 | 360 aa | 35.0 | 1.63 |
| ChdeLPAAT2 | Chlorella desiccata | KAH7617329.1 | 372 aa | 34.7 | 1.71 |
| ApLPAAT2 | Auxenochlorella | XP_011401335.1 | 385 aa | 34.7 | 1.58 |
| | protothecoides | | | | |
| ChsoLPAAT2 | Chlorella sorokiniana | PRW57325.1 | 363 aa | 33.8 | 1.53 |
| ChvaLPAAT2 | Chlorella variabilis | XP_005852243.1 | 357 aa | 33.2 | 1.58 |
| ChprLPAAT2 | Chloropicon primus | QDZ21586.1 | 335 aa | 28.8 | 1.62 |
| BpraLPAAT2 | Bathycoccus | ERR2041002 | 334 aa | 27.9 | 1.66 |
| | prasinos_ERR2041002 | | | | |
| OlLPAAT2 | Ostreococcus lucimarinus | XP_001421357.1 | 286 aa | 25.3 | 1.63 |
| VrLPAAT2 | Volvox reticuliferus | GIM16764.1 | 156 aa | 25.0 | 1.69 |
| PproLPAAT2 | Pycnococcus | ERR2041000 | 326 aa | 23.2 | 1.64 |
| | provasolii_ERR2041000 | | | | |
| CosLPAAT2 | Coccomyxa subellipsoidea | XP_005647638.1 | 197 aa | 22.9 | 1.63 |
| NoLPAT4 | Nannochloropsis oceanica | BAW99690.1 | 445 aa | 16.2 | 1.55 |
| CrLPAAT1 | Chlamydomonas | XP_042921325.1 | 332 aa | 12.9 | 1.59 |
| | reinhardtii | | | | |
| CrLPAAT1- | Chlamydomonas | XP_042921325.1 | 302 aa | 12.9 | 1.69 |
| ΔCST* | reinhardtii | | | | |
| AGPAT1 | Homo sapiens | NP_001358366.1 | 283 aa | 12.6 | 3.6 |
| NoLPAT3 | Nannochloropsis oceanica | BAW99691.1 | 482 aa | 12.1 | 1.6 |
| EcplsC | Escherichia coli | HBN6599230.1 | 245 aa | 10.9 | 1.59 |
| BnBAT2-ΔCST* | Brassica napus | NP_001302928.1 | 258 aa | 10.3 | 1.9 |
| Sll1848 | Synechocystis sp. | WP_010873224.1 | 225 aa | 10.3 | 1.6 |
| | PCC6803 | | | | |
| BnBAT2 | Brassica napus | NP_001302928.1 | 344 aa | 10.3 | 1.57 |

Table 112 shows the amino acid sequences of LPAATs described herein.

In some embodiments, a microalgal cell provided herein comprises an exogenous gene that encodes for a LPAAT in Table 112. In some embodiments, the exogenous gene can comprise a sequence that encodes for an enzyme with at least 50%, at least 60%, at least 70%, at least 80%, at least 90%, at least 95%, at least 96%, at least 97%, at least 98%, at least 99%, or 100% sequence identity to a LPAAT in Table 112. In some embodiments, the exogenous gene can comprise a sequence that encodes for an enzyme with at least 50%, at least 60%, at least 70%, at least 80%, at least 90%, at least 95%, at least 96%, at least 97%, at least 98%, at least 99%, or 100% sequence identity to any one of SEQ ID NO: 83-106, 155, and 156.

TABLE 112
LPAAT Amino Acid Sequences

| SEQ ID | | |
|---|---|---|
| NO: | LPAAT | Amino Acid Sequence |
| 83 | VcomLPAAT2 | MSLISKALSLPSFLLSVFVFYWSLPIFAIPYRIRFANVGKR |
| | | NDMLDWAKALFAFFRVTVLKQGEQGLYRGGPCLYLCN |
| | | HRSWADFFVDVYLTEGRAALMSRWLVFFVFPVFCTSCL |
| | | ILRGIVLFKRGTILDKEAFNAWLDATLRRSTVPGLLIYPE |
| | | GHRSLKPTSLPLKRGMLHFAYSRRLPVQLIVTRGKEDIL |
| | | SEKTMAMKYGRTLVTTYSKVLKPTDYDSFDAFMGDVQ |
| | | TSWDAGWKASYEAPTKGVPQLSMAGAHEYDYPATMR |
| | | WQQLAITLFSIVVFAAVLYGTWWGLGAVLAAAGPLGQ |
| | | QVVVVLAATSWLGASLIRSCR |
| 84 | VitrLPAAT2 | MSLISKASSLPSFLFSVFVFYWSLPIFAVPYRIRFANMGK |
| | | RNDMLDWAKALVAFFRVTVLKQGEQGLYRGGTCLYL |
| | | CNHRSWADFFIDVYLTEGRAALMSRWLVFFVFPVFCTS |
| | | CLILRGIVLFKRGTILDKEAFNAWLDATLRRSIVPGLVV |
| | | YPEGHRSLKPTSLPLKRGMLHFAFGRQLPVQLIVTRGKE |
| | | DVLSEKTMAMKYGRTLVTSYSKVLKPSDYASFDAFMT |
| | | DIQTCWDSCWRAAYEAPTKGVPQLSMVGAHEYDYPRT |
| | | IWWQQLWITLASILIFAGVLYGTWRGLASLLAAATTSG |
| | | ALGPQVLVTVAAAWAGSSLLRSLL |
| 85 | CchaLPAAT2 | MSIVTKVLNLPSFLFSVFVFYWSLPLFAILYRIRFANVGK |
| | | RNDMLDWAKTLVGFFHVTILKQGELSLYRDGPCLYLC |
| | | NHRSWADFFIDTYLTEGRAALMSRWLVYFVFPVFCTSV |
| | | IILRGIVLFKRGSIADKEAFNAWLDATLRKSIVPALLVYP |
| | | EGHRSLRPTSLPLKRGMLHYAYSRRLPVQLIVTSGKEA |
| | | VMSEKTMSVRFGRTLVTTYSKVLRPADYPTFDAFFSDI |
| | | QATWDGCWDAAYKQPSAGAPQLRLVDPHGYNYPARM |
| | | WWLQLGVTLLSIGVMAAVIAASWWGLGALLAATGPA |
| | | KQVVLGVVVSLLGASLLRSCL |
| 86 | PjapLPAAT2 | MSVIYKALSLPSFLFSVFVFYWSLPLFASVYRIRFANFGK |
| | | RNDMLDWAKALVAFFRVTVLKQGEMSLYRGGPCLYL |
| | | CNHRSWADFFIDTYLTEGRAALMSRWLVYFVFPVFCTS |
| | | VLILRGIVLFKRGHIVDKEAFNAWLDATLFKSTVPGLLV |
| | | YPEGHRSLKPASLPLKRGMLHFAFSRKLPVQVICTRGKE |
| | | EVLCEKTLSARWGRTLVVTFSKVIKPSEYDSFDAFFNDV |
| | | QSTWDACWQLSYHQPTAGVPELSMKDAHEYDYPRSM |
| | | WWLQLGITLLSFIILAAVLWGSWVAVRAGFATLGSVQQ |
| | | AVVLGTAGWIGASMVCGFL |
| 87 | VbolLPAAT2 | MSALKKALSLPSFLFSVFVFYWSLPIFASVYRIRFARAG |
| | | KRNDMLDWATSLVAFFRVTVLKQGQFSIYRGGPCIYLC |
| | | NHRSWADFFIDMYLTEGRAAFMSRWMVFFVFPVFCSA |
| | | VLILKGIILFKRGRILDKEAFNAWLDATLAKSTIPGLVVY |
| | | PEGHRSLKPTSLPLKRGMLHYAFSRKLPVQLIVTRGKEE |
| | | VLSEKTLSMRFGRTLVTSYSKVMKPSDYGSFDAFMTDV |
| | | QSGWDQCWKEAYSAPTHGLPQLSMAGATEYDYPASM |
| | | RWQHLAMTLFSIVVFAGVLAASWWGVGALLSAAGPTG |
| | | KQVVVLLASCWFGASLLRSFL |
| 88 | PmorLPAAT2 | MSLVTKFLSLPSFLFSVFIFYWSLPIFAIPYRIRFANVGKR |
| | | NDMLDWAKALVGFFRVTVLKPGATSVYRGGPCLYLCN |
| | | HRSWADFFIDVYLTQGQSALMSRWLVFFVFPVFCTSCLI |
| | | LRGIVLFKRGTILDKEAFNAWLDATRARSIVPGLVVYPE |
| | | GHRSLKPTSLPLKRGMIHYAFSRRLPVQLIVTSGKEHVL |
| | | SEKTMSVKFGKTLVTSYSKPLKPSDYATFDAFMADIQS |
| | | NWDQCWKEAYTAPARGMPEVSMAGAHSYAYPRLTW |
| | | CLQLAITVVSIVILGGVLAGSWRGAAAVLAAAGPVGQQ |
| | | VLVSLLALWAGVSLLRSFL |
| 89 | VcarfLPAAT2 | MSITSKALSLPSFLFTVFVFYWSLPLFAIIYRIRFANVGKR |
| | | NDMLDWAKALVAFFRVTVLKQGEMSLYRGGRCLYLC |
| | | NHRSWADFFIDTYLTEGRAALMSRWMVFFAFPVFSTSV |
| | | LILRGIVLFKRGHIADKEAFNSWLDATLRKSTVPALLVY |
| | | PEGHRSLKPRSLPLKRGMLHFAFSRQLAVQMVITRGKE |
| | | EVLSEKTCSARWGRTLVTTYSKVIKPTDYDSFDAFFNEI |
| | | QTTWDACWQLSYHQPTEGVPQLSLKGAHEYDYPVNM |
| | | WWLQLGVTLLSITILVAVLYGSWVALRAGVSSLGSSIT |
| | | QQAVVLGVAGWLGVSLLRSFL |
| 90 | EcylLPAAT2 | MSLISKALSLPSFLFSVFVFYWSLPFFATTYRIRFANVGK |
| | | RNDMLDWAKTLVAFFRVTVLKQGNMSLYRGGRCLYL |
| | | CNHRSWADFFIDTYLTEGRAALMSRWLVYFVFPVFCTS |
| | | VIILRGIVLFKRGHIADKEAFNAWLDATFSRSIVPGLLVY |
| | | PEGHRSLKRTSLPLKRGMMHYAYSRRIPVQLIVTRGKE |
| | | EVLSEKTLSARWGRTLVTTYSKVLKPSDYDSFDAFFNDI |
| | | QAQWDACWKTSYDTPAAGVPQLSMVDAHEYDYPRRM |
| | | WWLQLGMTLLSIVILAAVLWGSWLGIRAGLATLGSLQ |
| | | QQAVVWGASGWLGASLLLSFF |
| 91 | GmulLPAAT2 | MGAAGKLLSLPSFLFSVFVFYWSLPIFALVYRIRFASSGR |
| | | RNDMLDWAKSIVRFFGVTVVKVGENSLYREGPCLYLC |
| | | NHRSWADFFIDAYLTEGRAALMSRWLVFFVFPVFCTSC |
| | | LILRGIVLFKRGTIADKEGFNRWLDSKLGSSHVPGLLVY |
| | | PEGHRSLRPSSLPLKRGMLHYAYGRKLAVQIICTAGKE |
| | | GVMSEKTMAVHFGRTLVTTFSQVLRPSEYPTFDAFFAA |
| | | VQSAWDASWKAANQTPSAGLPRLSMEGAQDYDYPPA |
| | | MRAQQLGVTLLSFVVFGGALLVTWRLLAALLAAAGAA |
| | | QQAVALLLAGWTGVSMLRSFL |
| 92 | GvirLPAAT2 | MGAASKLLGLPSFLFSVFVFYWSLPIFAIVYRIRFASPGR |
| | | RNDMLDWAKSIVGFFGVAVHKAGTNSLYRDGPCLYLC |
| | | NHRSWADFFIDAYLTEGRAALMSRWLVFFVFPVFCTSC |
| | | LILRGIVLFKRGTIADKEAFNSWLDATLGRSIVPGLLVYP |
| | | EGHRSLRPASLPLKRGMLHYAFGRKLPVQVVCTAGKE |
| | | GVMSEKTMAVRFGRRLVTNFSQVIRPADFPTFDAFSAA |
| | | VQSAWDGCWKAAYETPAAGLPRLSMEGSHEYEYDYP |
| | | APMRVLQVCITLLSIVLFLGVLLGSWRALAAALAAAGA |
| | | AQQVVVALLATWLGASMLRSCL |
| 93 | VferLPAAT2 | MSFISKAVGLPSFLFSVFVFYWSLPIFALTYRIRFASKGK |
| | | RNDMLEWAKALVRFFGVKVLTQGEHSLYRGGPCLYLC |
| | | NHRSWADFFIDAYLTEGRAALMSRWLVYFVFPIFCTSV |
| | | MVLRGIVLFKRGTIADKEAFNAWLDAKMKSSTIPGLLV |
| | | YPEGHRSLKRTSLPLKRGMLHFAFSRGLPVQLVMCAGK |
| | | EEVLSEKRMAVRFGRTLVATYSEVLKPGNYPTFDAFFS |
| | | GIQTAWDSCWQAAYKQPPEGAAPLPMVNTTEYDYPPR |
| | | MRRLQLIVTLLSIVVLAAVLAGSVLVTVVAAAAVGPMP |
| | | AGVVVAVLASWLGGSMLRSFL |
| 94 | VaulLPAAT2 | MGAASKVLGLPSFLFSVFVLYWSLPIFAVLYRIRFAKMI |
| | | GKRNDVLDWAKSLVRFFGITVLRQGQEQLYTGGPCLY |
| | | LCNHRSWADFFLDVYLTEGHAAVMSRWLVYFVFPVFC |
| | | TSCLILKTIVLFKRGTIRDKEGFNRWLDAVLARSNVPGL |
| | | LVFPEGHRSLRPASLPLKRGMLHYAYGRKLAIQMVVTR |
| | | GKEEVMSEKTMAVRFGRTLVTTYSKVLRPADYPDFEAF |
| | | FADLQSTWDGCWEAAYRKPREDAVRHSLGAAPGFHYP |
| | | FATRVAQLGATLGSIALFGLVLVGCWRGLAAALAATG |
| | | AAQQAVAALLAVWVGGSMLRSFL |
| 95 | Ch_CCAC2762_ | MRQLKSLIQLPYFLFCVFVFYWSFPIGCLTHHIHFLKFSG |
| | LPAAT2 | KRNDMHTWSETMCSFFGIRFVKYGEEKLSKGSRNILYL |
| | | SNHRSWADFFVDVYLTEGNAAMLSRMLVFFVFPIFMTS |
| | | VVVLKGIILFKRGTIADKEKFNAWLDVMLERSPIPGMLV |
| | | YPEGHRSTRPHSLPLKRGMLYYAHSRGMPVQIIISNGKE |
| | | GVMSEKQRRAGFGKTVVVGFSSVIDTAAYKGNVEGFM |
| | | ARVQEEWNKLWGQVYSADPAGLPPLRQDPPMLDYAA |
| | | SMRTQQLAIAVFSMFALAGVLWVTWRMFAWLTALAG |
| | | PLQHALAVLLLTWFAASIIAGVMGPSLPTHSSSLQNPPS |
| | | LKADKSVSAAAAAAAAASGDGSLHEAPSGKAPAAAAT |
| | | QNHNHQH |
| 96 | DsalLPAAT2 | MGQSTLQKLISLPTFFLSIFLMYWSLPLCTLCGYLRLLDF |
| | | KSTRKDMYNWCRFMTRLFRIKVKKYGSKSLRKDAGRH |
| | | ILYLCNHRSWTDFFLDAYLTEGNASLMSRWLVFAVFPV |
| | | FIVSVIVLRGVILFKRGTILDKEKFNRWLDSKILSTPIPGV |
| | | LVYPEGHRSLADKSLPLKRGMLYYAHSRKFPVQIIISRN |
| | | KEDVLSEKTMKAHFGATIITGCSDLIESDGKDIKVFLSEI |
| | | QAAWDDLWEKVYAADATALPPLEQSNIPEFDYSMGMR |
| | | VKQLLVVCSSVLVFLAAMVVFGKLSCLIYAAMGDTER |
| | | KVLVTLILYVWFFVSMLNSRAPSATQ |
| 97 | MyarcLPAAT2 | MSAEKLLHAPSFVITCLVLYWSLPLSVLFGYIRAFQWTG |
| | | PRNDMYSWGQFMSDFFGVKFVRVGNEKLYKPKKGAN |
| | | VIYLCNHRSWADFFCDVYLTQGNAALLSRMLVFFIFPFF |
| | | MSSVVILKGVVLFKRGKVVDKDAFNKRLDGKLADSPIP |
| | | GLLVYPEGTRSLKDLSLPLKRGMLYFAHSRNIPVQIVVS |
| | | SGKEYVLTEKKIRARIGRTVVSTFSAKITPSDFPSCEAFL |
| | | EHVQKEWDRLWPAVFTADADKAPVLVNEDIPMHDYA |
| | | NTMRWAQLGMAVLSTTVATIMELLSWKVVDLIFGIFGP |
| | | LKQVLYMLLFTWVSVSLVASFLGPKHLPKGVLPSPVLP |
| | | LPVARPPPPSQEPSSSGSK |
| 98 | CuwoLPAAT2 | MAANMATFPFFLLTFFFLYWSMAIGTLVGRVKFWQYN |
| | | GTNRNEMFVWAKLCSDFLGVKYLKTGSGSLYKKERCV |
| | | YLSNHRSWADFFVDVYLTEGIAQAMARTMVGIAFPMFI |
| | | GAATILRGILLFKRGSIANKDKFNTWLDDKLNSSPCSSL |
| | | LVFPEGHRSTKSTSLPLKRGMLYYAHSRGLPVQVIMTR |
| | | GKELVLSEKHHHAGYGTQLITGFSELVLSKDKEFEQFW |
| | | TEVQAMWDRMWAEVYSVDPSTAVPYTQDVPGSVYPL |
| | | SMSVLYTVFTVSTVIFLLSTLWIMARIFGFMISPLPAMLQ |
| | | MVVWGAVPVTLAVSFRASIIDNAKTSSSAVIKTAKKAQ |
| 99 | CmoeLPAAT2 | MASFVDLLLQFPYFGLTIFFFYWSPPLASIVYRIRFASAG |
| | | PRKDIYEWTTAMVQFLGVRIRHQGKQTLYKKERCVYL |
| | | CNHRSWADFFVDLHLTQGLAAPMSRTMVFFAFPVFMSI |
| | | CIVLKGVLLFKRGTIADKTKFNKWLDDSLGNSPVSCLL |
| | | VYPEGHRSTKAKSLPLKRGMLYYAHSRGLPVQIIHTAG |
| | | KEAVLSEKRMRASYGQTLVTGFSQPIQSKDFKDFEAFS |
| | | AEVQRSWDALWTEVYSANPSALPPLVQEDVPTADYPPS |
| | | MRTGMLLVSLAAIALFLAFVALLAYITGPVMSLFPAPLR |
| | | VPLILFKLSWHAASFLYCRAKDTPGKVKAA |
| 100 | OambLPAAT2 | MALARSSSSSALLLWLLRLPYFLLTITFFYWSATIGALIY |
| | | RVRFLSTGPRNDMYDWTLLMTSFLGLHFHQLGAQRLH |
| | | CAPRVVFLANHRSWADFFVDIHLTQGTAAPMSRALVAF |
| | | AFPVFMCTAWLLRGLIIFKRGAIEDKKKFNSWLDEQLD |
| | | ASAGSNLLVYPEGHRSTRPTSLPLKRGMLYYAHSRGLP |
| | | VQIIMTAGKESVLSEKHQCASFGQHLLTSFSPVISSKDK |
| | | DFEVFATEVQQRWDSLWREVFSTSTQGVPLLHQASVPL |
| | | FDYPPAVRTHMALASSAFILMLLLAAGAAVLAVRATLA |
| | | LMPPVLQVVVLAASSLLMLASFLTSAAITSSTTTTTTTT |
| | | RQPQAVADKGSKES |
| 101 | EdLPAAT2 | MSALNKALGLPSFLFSVFVFYWSLPIFATFYRIRFASNG |
| | (Edaphochlamys | KRNDVLDWAKSLVRFFGVTVLQQGEHSLYRGGACLYL |
| | debaryana_SRR | CNHRSWADFFIDAYLTQGLAALMSRWLVYFVFPVFCTS |
| | 13719274) | CMILRGVVLFKRGTIADKDAFNKWLDDTLGSSPVPGLL |
| | | VYPEGHRSIKPTSLPLKRGMLHYAYSRKKPVQIIITRGK |
| | | DEILSEKTLAVHFGRTVVTTFSKVLRPADYATFEAFFTE |
| | | LQSTWDSCWVAAYTTPIAGIPRLSMADPHAYDYSSTMR |
| | | AQQLAITLFSILVLVGVLAGSWRVWTAVLAATGTAQTV |
| | | VVLVLASWVGTSMLRSFL |
| 102 | VsteLPAAT2 | MSFLGKALSFPAFLFSVFVFYWSLPIFASVYRIRFVKRG |
| | | KRNDMLDWAKSLVDFFRVRVLKQGENSLYRGGRCLYL |
| | | CNHRSWADFFLDMYLTEGRAAFMSRWMVFFVFPVFCS |
| | | ACLVLRGIVFFKRGIIRDKEAFNAWLDATLSRSIVPGLV |
| | | VYPEGHRSLRPTSLPLKRGMLHFAFDRRLPVQVIVTSGK |
| | | EGVLSEKTLAMRFGCTLVVSYSKVLKPSEYDTFEAFMS |
| | | DVQAQWDTCWKAVYGSPTTGAPELSMVGAHQYDYPL |
| | | SMRVQHVAMTLLSIGIMGAVVIGSWRGWNAVLLALGG |
| | | PVVQQAVVLLGAAWLGVSLLRSCL |
| 103 | GoctLPAAT2 | MTTMREEKWHTQQADVGAKSTASKAERVGATSKLLG |
| | | FPSFLFSVFVLYWSLPIFAILYQIRFASPGRRNNSLDWAK |
| | | SLVGFFGVTVVKTGEDSLYRGGPCLYLCNHRSWADFFI |
| | | DAYLTEGRAALMSRWLVFFVFPVFLTAVRIIRGVVLFK |
| | | RGAVADKEAFNAWLDATLKRSVVPGLLVYPEGHRSLR |
| | | PTSLPLKRGMLHFAYGRKLPVQIIVTAGKESVMSEKAL |
| | | AVHFGRALVTKFSRALRPTDYPGFDAFFAAVQSTWDEC |
| | | WKAAYGTPAAGTPRLTIDGAHGYDYPPLMRWQLLVVT |
| | | LLCILLFGGVLLASWRALAALLAAAGAARRAVVLLLAL |
| | | WVGASASRSFV |
| 104 | CnivLPAAT2 | MARGSGSIGRRPGWLSLLLDLPAFLLAIFILYWSSALGA |
| | | ALGYIRFTKMRGLRDDTFEWSKFMLHAFAVRIVRLGPV |
| | | LPYRQSPCMYLCNHRSWADFFVDTYCTEGRSALMSRW |
| | | LVWWVFPVFMSVCRIIGGVVLFKRGAVFDKEKFNAWI |
| | | DKRLTEVPGHSLLVFPEGHRSISSSSLVLKRGMLHYAHS |
| | | RKLPVQVVLSAGKEGVLSETRQCVRFGRTVVVTYADVI |
| | | KSGDFPNFDDFLATVQSTWEEKWRQVHSAPKDGLPAL |
| | | DPVGMSFEYTPTTRWRHVAATLSSYVFMAVVLYASWQ |
| | | AAAYALLAMGPLAPVISTILVVWSAVSLTLAWLPERTP |
| | | SARHWAAPATNGPVASSPVD |
| 105 | PproLPAAT2 | MSLVTEILLRITFCVDLFLLQIVIPLLALLYSPRDAGTHN |
| | | MSAWTSKMLGYRIHKAGQNTCYVPEEKSTRVMYLAN |
| | | HRSWGDFIVDRLLTGFRGGSLSRIMVIVGIPMPATLAWL |
| | | GSGQRGTSALWFFKRAKGATPALWATLDRNFEVAIEK |
| | | AVVVYPEGHRNRSDKPLPIRTGTLRYAFDRNVHVQVLI |
| | | AEGKERVINEAQLVTRRSPNVVTVSFSDPIVPKNYSTFE |
| | | GFHGAVCEAWRTTWLAAYGAAAEAGYCAPPPALGPD |
| | | GMAESATAADHKRIHRPPAAVPWTPPNPGPPPNQHASL |
| | | GMHMARLCIVGAISAAAYYTLYE |
| 106 | BpraLPAAT2 | MGLSSPSKKLESPSSSRDKYVDTRSLAEKGVHLIALLIAF |
| | | PILAFSYPLSLLTRGTLHGVWNFSKEIRPPITQIWADFMT |
| | | SNVMNVRFKIVSQKQLYRDGKCVYLCNHRAWADFFID |
| | | MSLCEGRSFTLSRMLVMYVFPVFMVPAVFVGAVFPFRR |
| | | DKGGGHEELNNSIDKHWSRFEQYFPGFLCYPEGTRNIKP |
| | | HSLPLKRGMLRYAHSRKLPVQIIICAGKEKVLSQKRFAA |
| | | ERNHVLPVAYSSVIHSDSYPVFEDFAEAIRHSWADEWA |
| | | NAYNHENKMEKLKDFKPSERTIRYERWVDFFTTGTVVS |
| | | FFVFCALVANVLQIIFPKIIELGHA |
| 155 | CrLPAAT1- | MARKSSLAQAAPVAPSPSAKPELPARSALVCHAAAASV |
| | ACST* | PLPNSDSAPQPNVLLAKIRAIMFFAWSFLLSLPLFVTMM |
| | | VMAPLVLAFDKYRRLAQHFVNNLWACASTAPFYKVTII |
| | | GRENLPPPDKPVVYVANHQSFLDIYSLFHLQRPFKFISKT |
| | | SNFLIPIIGWSMFLTGHVMINRVDRRSQLKCLQQCRDLL |
| | | AEGAPVLFFPEGTRSLDCKMAGFKKGAFSVAAKAGVE |
| | | VVPITLLGTGSLMPSGKESQLRPGQVTIVVHKALPPNKN |
| | | ADQLCDAARQAVASSLPPELVGSATEMAPDEQ |
| 156 | BnBAT2-ACST* | MSDLSGAATPESTYPEPEIKLSSRLRGICFCLVAGISAIVL |
| | | IVLMIIGHPFVLLFDRYRRKFHHFIAKLWASISIYPFYKT |
| | | DIQGLENLPSSDTPCVYVSNHQSFLDIYTLLSLGQSYKFI |
| | | SKTGIFVIPVIGWAMSMMGVVPLKRMDPRSQVDCLKR |
| | | CMELVKKGASVFFFPEGTRSKDGRLGPFKKGAFTIAAK |
| | | TGVPVVPITLMGTGKIMPTGSEGILNHGDVRVIIHKPIYG |
| | | SKADVLCEEARNKIAESMNLLS |

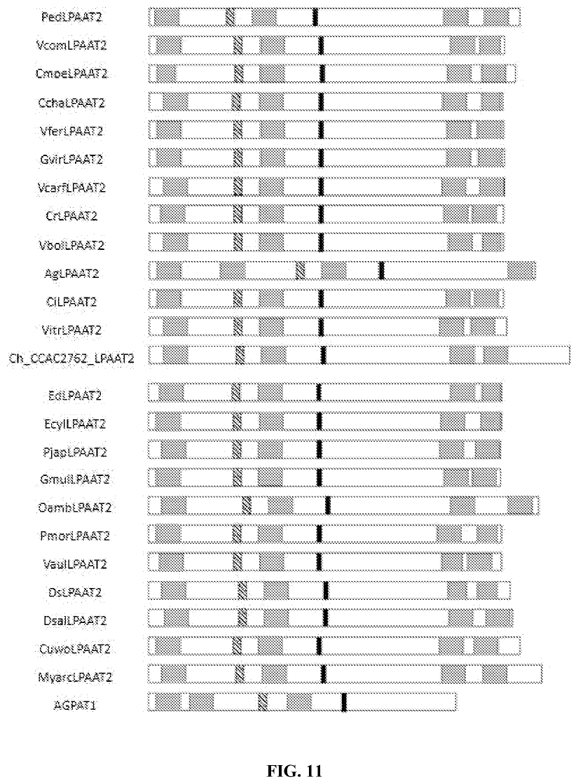

Table 113 shows conserved LPAAT motifs and the percent identity (%) of the most active LPAATs across the entire LPAAT protein relative to PedLPAAT2. The transmembrane domains of these LPAATs were compared and the schematic diagrams of the LPAATs are shown in FIG. 11. The LPAAT catalytic domain NHX₄D is shown in shaded bar, binding motif EGTR or EGHR is shown in black bar, and transmembrane domains are indicated by gray bar.

TABLE 113
Percent identity against PedLPAAT2 and LPAAT motifs

| | Percent | LPAAT Motifs | | | |
|---|---|---|---|---|---|
| | identity | I | II | III | IV |
| Gene name | (%) | (NHX₄D) | ((F/Y)XXR) | (EGXR) | (Proline) |
| PedLPAAT2 | 100 | NHRSWAD | GIVLFKRG | LLLYPEGTRNT | PFQIIVTTNKE |
| | | (SEQ ID NO: | (SEQ ID | KP (SEQ ID NO: | (SEQ ID NO: |
| | | 107) | NO: 110) | 120) | 135) |
| VcomLPAAT2 | 45.882 | NHRSWAD | GIVLFKRG | LLIYPEGHRSL | PVQLIVTRGK |
| | | (SEQ ID NO: | (SEQ ID | KP (SEQ ID NO: | E (SEQ ID NO: |
| | | 108) | NO: 111) | 121) | 136) |
| CmoeLPAAT2 | 45.882 | NHRSWAD | GVLLFKR | LLVYPEGHRST | PVQIIHTAGK |
| | | (SEQ ID NO: | G (SEQ ID | K (SEQ ID NO: | E (SEQ ID NO: |
| | | 108) | NO: 112) | 122) | 137) |
| CchaLPAAT2 | 43.824 | NHRSWAD | GIVLFKRG | LLVYPEGHRSL | PVQLIVTSGK |
| | | (SEQ ID NO: | (SEQ ID | R (SEQ ID NO: | E (SEQ ID NO: |
| | | 108) | NO: 111) | 123) | 138) |
| VferLPAAT2 | 43.529 | NHRSWAD | GIVLFKRG | LLVYPEGHRSL | PVQLVMCAG |
| | | (SEQ ID NO: | (SEQ ID | K (SEQ ID NO: | KE (SEQ ID |
| | | 108) | NO: 111) | 124) | NO: 139) |
| GvirLPAAT2 | 43.529 | NHRSWAD | GIVLFKRG | LLVYPEGHRSL | PVQVVCTAG |
| | | (SEQ ID NO: | (SEQ ID | R (SEQ ID NO: | KE (SEQ ID |
| | | 108) | NO: 111) | 123) | NO: 140) |
| VcarfLPAAT2 | 43.529 | NHRSWAD | GIVLFKRG | LLVYPEGHRSL | AVQMVITRG |
| | | (SEQ ID NO: | (SEQ ID | K (SEQ ID NO: | KE (SEQ ID |
| | | 108) | NO: 111) | 124) | NO: 141) |
| CrLPAAT2 | 43.235 | NHRSWAD | GIVLFKRG | LLVYPEGHRST | PVQIVVTRGK |
| | | (SEQ ID NO: | (SEQ ID | K (SEQ ID NO: | D (SEQ ID NO: |
| | | 108) | NO: 111) | 122) | 142) |
| VbolLPAAT2 | 43.235 | NHRSWAD | GIILFKRG | LVVYPEGHRSL | PVQLIVTRGK |
| | | (SEQ ID NO: | (SEQ ID | K (SEQ ID NO: | E (SEQ ID NO: |
| | | 108) | NO: 113) | 125) | 136) |
| AgLPAAT2 | 42.941 | NHRSWAD | GVIFIDRK | LLVYPEGHRSL | PVQIIITRGKE |
| | | (SEQ ID NO: | (SEQ ID | K (SEQ ID NO: | (SEQ ID NO: |
| | | 108) | NO: 114) | 124) | 143) |
| CiLPAAT2 | 42.941 | NHRSWAD | GIVLFKRG | LLVYPEGHRST | PVQVIITRGK |
| | | (SEQ ID NO: | (SEQ ID | K (SEQ ID NO: | D (SEQ ID NO: |
| | | 108) | NO: 111) | 122) | 144) |
| VitrLPAAT2 | 42.941 | NHRSWAD | GIVLFKRG | LVVYPEGHRSL | PVQLIVTRGK |
| | | (SEQ ID NO: | (SEQ ID | K (SEQ ID NO: | E (SEQ ID NO: |
| | | 108) | NO: 111) | 125) | 136) |
| Ch_CCAC2762_ | 42.941 | NHRSWAD | GIILFKRG | MLVYPEGHRS | PVQIIISNGKE |
| LPAAT2 | | (SEQ ID NO: | (SEQ ID | TR (SEQ ID NO: | (SEQ ID NO: |
| | | 108) | NO: 113) | 126) | 145) |
| EdLPAAT2 | 42.647 | NHRSWAD | GIILFKRG | MLVYPEGHRSI | PVQVIITRGK |
| | | (SEQ ID NO: | (SEQ ID | K (SEQ ID NO: | D (SEQ ID NO: |
| | | 108) | NO: 113) | 127) | 144) |
| EcylLPAAT2 | 42.353 | NHRSWAD | GIVLFKRG | LLVYPEGHRSL | PVQLIVTRGK |
| | | (SEQ ID NO: | (SEQ ID | K (SEQ ID NO: | E (SEQ ID NO: |
| | | 108) | NO: 111) | 124) | 136) |
| PjapLPAAT2 | 42.353 | NHRSWAD | GIVLFKRG | LLVYPEGHRSL | LPVQVICTRG |
| | | (SEQ ID NO: | (SEQ ID | K (SEQ ID NO: | KE (SEQ ID |
| | | 108) | NO: 111) | 124) | NO: 146) |
| GmulLPAAT2 | 42.059 | NHRSWAD | GIVLFKRG | LLVYPEGHRSL | AVQIICTAGK |
| | | (SEQ ID NO: | (SEQ ID | R (SEQ ID NO: | E (SEQ ID NO: |
| | | 108) | NO: 111) | 123) | 147) |
| OambLPAAT2 | 41.471 | NHRSWAD | GLIIFKRG | LLVYPEGHRST | PVQIIMTAGK |
| | | (SEQ ID NO: | (SEQ ID | R (SEQ ID NO: | E (SEQ ID NO: |
| | | 108) | NO: 115) | 128) | 148) |
| PmorLPAAT2 | 41.176 | NHRSWAD | GIVLFKRG | LVVYPEGHRSL | LPVQLIVTSG |
| | | (SEQ ID NO: | (SEQ ID | K (SEQ ID NO: | KE (SEQ ID |
| | | 108) | NO: 111) | 125) | NO: 149) |
| VaulLPAAT2 | 40 | NHRSWAD | GIVLFKRG | LLVFPEGHRSL | AIQMVVTRG |
| | | (SEQ ID NO: | (SEQ ID | R (SEQ ID NO: | KE (SEQ ID |
| | | 108) | NO: 111) | 129) | NO: 150) |
| DsLPAAT2 | 40 | NHRSWTD | GVILFKR | MLVYPEGHRS | PVQIIISRNKE |
| | | (SEQ ID NO: | (SEQ ID | LA (SEQ ID NO: | (SEQ ID NO: |
| | | 109) | NO: 116) | 130) | 151) |
| DsalLPAAT2 | 39.412 | NHRSWTD | GIVLFKRG | VLVYPEGHRSL | PVQIIISRNKE |
| | | (SEQ ID NO: | (SEQ ID | A (SEQ ID NO: | (SEQ ID NO: |
| | | 109) | NO: 111) | 131) | 151) |
| CuwoLPAAT2 | 39.412 | NHRSWAD | GILLFKRG | LLVFPEGHRST | PVQVIMTRGK |
| | | (SEQ ID NO: | (SEQ ID | K (SEQ ID NO: | E (SEQ ID NO: |
| | | 108) | NO: 117) | 132) | 152) |
| MyarcLPAAT2 | 38.529 | NHRSWAD | GVVLFKR | LLVYPEGTRSL | PVQIVVSSGK |
| | | (SEQ ID NO: | G (SEQ ID | K (SEQ ID NO: | E (SEQ ID NO: |
| | | 108) | NO: 118) | 133) | 153) |
| AGPAT1 | 12.647 | NHRSWAD | GIVLFKR | WVFPEGTRNH | PIVPIVMSSY |
| | | (SEQ ID NO: | (SEQ ID | N (SEQ ID NO: | (SEQ ID NO: |
| | | 108) | NO: 119) | 134) | 154) |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An oil comprising:
at least 10 mg of ergosterol per 100 g of the oil; and
a triacylglyceride (TAG) component,
wherein at least 40% of TAGs in the TAG component are TAG species having a saturated fatty acid at the sn-2 position,
wherein at least 50% of the acyl chains in the TAG component are C18:1.

2. The oil of claim 1, wherein the saturated fatty acid is C16:0.

3. The oil of claim 1, wherein at least 50% of the TAGs in the TAG component are TAG species having a saturated fatty acid at the sn-2 position.

4. The oil of claim 1, wherein the TAG species comprise C18:1 at the sn-1 and sn-3 positions.

5. The oil of claim 1, wherein the TAG species comprise C16:0 at the sn-2 position.

6. The oil of claim 1, wherein the TAG species comprises or consists of 1,3-dioleolyl-2-palmitoyl glycerol (OPO).

7. The oil of claim 1, wherein at least 20% of the acyl chains in the TAG component are C16:0.

8. (canceled)

9. (canceled)

10. The oil of claim 1, wherein the oil has an OP:OO m/z ratio of at least 1.6 as determined by abundance of diacylglycerol (DAG) ions resulting from mass spectrometry fragmentation of the TAG component in the oil.

11-13. (canceled)

14. The oil of claim 1, wherein the oil comprises at least 50 mg of ergosterol per 100 g of the oil.

15. (canceled)

16. (canceled)

17. The oil of claim 1, wherein the oil comprises no more than 5 mg of campesterol, no more than 5 mg of β-sitosterol, or no more than 5 mg of stigmasterol per 100 g of the oil.

18. (canceled)

19. (canceled)

20. The oil of claim 1, wherein the oil comprises no more than 5 mg of stigmasterol per 100 g of the oil.

21-23. (canceled)

24. The oil of claim 1, wherein the oil further comprises one or more of ergosta-5,8-dien-3-ol, (3β)-, 5.xi.-ergost-7-en-3β-ol, 9,19-cyclolanostan-3-ol,24-methylene-,(3β)-, and ergosta-7,22-dien-3-ol, (3β).

25-30. (canceled)

31. The oil of claim 1, wherein the oil is an algal oil.

32. The oil of claim 1, wherein the oil is a genetically modified algal oil.

33. (canceled)

34. (canceled)

35. The oil of claim 1, wherein the oil is produced from a Prototheca cell.

36-42. (canceled)

43. A TAG polyol produced from the oil of claim 1.

44. A method for producing a TAG polyol, the method comprising:
a) subjecting the oil of claim 1 to epoxidation, thereby generating an epoxidized oil; and
b) ring opening the epoxidized oil in the presence of an alcohol, an acid, or hydrogen and a suitable catalyst, thereby generating the TAG polyol.

45. The method of claim 44, wherein the epoxidized oil is ring opened in the presence of the alcohol.

46. The method of claim 44, wherein the epoxidized oil is ring opened in the presence of hydrogen and the suitable catalyst.

47. The method of claim 44, wherein the epoxidized oil is ring opened in the presence of the acid.

48. A method for producing a TAG polyol, the method comprising:
a) subjecting the oil of claim 1 to hydroformylation, thereby generating a hydroformylated oil; and
b) reducing the hydroformylated oil in the presence of hydrogen and a suitable catalyst, thereby generating the TAG polyol.

49. The TAG polyol produced by the method of claim 44.

50. A microalgal cell comprising an exogenous gene that encodes for an enzyme having lysophosphatidic acid acyltransferase activity,
wherein the cell produces an oil comprising a TAG component,
wherein at least 40% of TAGs in the TAG component are TAG species having a saturated fatty acid at the sn-2 position,
wherein at least 50% of the acyl chains in the TAG component are C18:1.

51. (canceled)

52. The microalgal cell of claim 50, wherein the enzyme is an algal lysophosphatidic acid acyltransferase (LPAAT).

53. (canceled)

54. (canceled)

55. The microalgal cell of claim 50, wherein the enzyme comprises a sequence with at least 70%, at least 85%, or 100% sequence identity to any one of SEQ ID NO: 107-154.

56-203. (canceled)

204. The microalgal cell of claim 50, wherein the enzyme is encoded by an exogenous gene and is codon-optimized for expression in a Prototheca strain.

205. The microalgal cell of claim 50, wherein the cell does not comprise an exogenous glycerol-3-phosphate acyltransferase (GPAT1).

206. The microalgal cell of claim 50, wherein the cell is from a Prototheca base strain.

207. The microalgal cell of claim 206, wherein the Protheca base strain is Prototheca moriformis base strain UTEX 1533, Prototheca wickerhamii, or Prototheca moriformis.

208. (canceled)

209. The microalgal cell of claim 50, wherein the cell is from a non-genetically modified Prototheca base strain that produces an oil having a fatty acid profile of at least 50% oleic acid and/or at least 30% palmitic acid.

210. (canceled)

211. (canceled)

212. (canceled)

213. The microalgal cell of claim 50, wherein the microalgal cell produces at least 50% lipid by dry cell weight.

214. (canceled)

215. A method of producing a non-naturally occurring oil, the method comprising cultivating a microalgal cell in a culture medium,
wherein the oil comprises a TAG component,
wherein at least 40% of TAG species in the TAG component have a saturated fatty acid at the sn-2 position,
wherein at least 50% of the acyl chains in the TAG component are C18:1.

216-219. (canceled)

220. The microalgal cell of claim 50, wherein the enzyme is selected from:
a) a lysophosphatidic acid acyltransferase (LPAAAT) that is selected from a Chlamydomonas reinhardtii (CrLPAAT1), Chlamydomonas incerta (CiPLAAT2), Chlamydomonas schloesseri (ChsLPAAT2), Volvox africanus (VaLPLAAT2), Nannochloropsis oceanica (NoLPAT3), Chlamydomonas reinhardtii (CrPLAAT2), Volvox carteri (VcLPAAT2), Nannochloropsis oceanica (NoLPAT4), Volvox carter f. nagariensis (VcLPAAT2), Astrephomene gubernaculifera (AgLPAAT2), Edaphochlamys debaryana (EdLPAAT2), Dunaliella salina (DsLPAAT2), Scenedesmus sp. (SceLPAAT2), Micractinium conductrix (McLPAAT2), Chlorella sorokiniana (ChsoLPAAT2), Chlorella variabilis (ChvaLPAAT2), Raphidocelis subcapitata (RsLPAAT2), Chlorella desiccate (ChdeLPAAT2), Auxenochlorella protothecoides (ApLPAAT2), Chloropicon primus (ChprLPAAT2), Homo sapiens (AGPAT1), Chlamydomonas eustigma (CeLPAAT2), Pedinophyceae (PedLPAAT2), Volvox reticuliferus (VrLPAAT2), Chlorella vulgaris (ChvuLPAAT2), Volvulina compacta (VcomLPAAT2), Vitreochlamys sp. CL-2021 (VitrLPAAT2), Colemanosphaera charkowiensis (CchaLPAAT2), Pleodorina japonica (PjapLPAAT2), Volvulina boldii (VbolLPAAT2), Pandorina morum (PmorLPAAT2), Volvox carterif. weismannia (VcarfLPAAT2), Eudorina cylindrica (EcylLPAAT2), Gonium multicoccum (GmulLPAAT2), Gonium viridistellatum (GvirLPAAT2), Volvox ferrisii (VferLPAAT2), Vitreochlamys aulata (VaulLPAAT2), Chlamydomonas sp. CCAC2762_B (ChCCAC2762_LPAAT2), Dunaliella salina (DsalLPAAT2), Microglena sp. YARC (MyarcLPAAT2), Chlamydomonas sp. UWO_241 (CuwoLPAAT2), Chlamydomonas moewusii (CmoeLPAAT2), or Oophila amblystomatis (OambLPAAT2);
b) a 1-acyl-sn-glycerol-3-phosphate acyltransferase that is selected from Synechocystis sp. (Sll1848), Escherichia coli (EcPlsC); or
c) an acyltransferase of Brassica napus (BnBAT2).

221. The microalgal cell of claim 220, wherein the enzyme comprises a sequence with at least 50%, at least 60%, at least 70%, at least 80%, at least 90%, at least 95%, at least 96%, at least 97%, at least 98%, at least 99%, or 100% sequence identity to a sequence selected from SEQ ID NO: 1-22, 25-62, or 65-100.
